(19) United States
(12) Patent Application Publication
(10) Pub. No.: US 2026/0218193 A1
(43) Pub. Date: Jul. 30, 2026

(54) MODIFIED MULTI-SEGMENTED ANTISENSE OLIGONUCLEOTIDES FOR USE

(71) Applicant: AusperBio Therapeutics Inc., Foster City, CA (US)

(72) Inventors: Guofeng CHENG, Foster City, CA (US); Cheng Yong YANG, Foster City, CA (US)

(21) Appl. No.: 19/146,769

(22) PCT Filed: Jan. 10, 2024
(86) PCT No.: PCT/CN2024/071514

§ 371 Date: Jul. 9, 2025

(30) Foreign Application Priority Data
Jan. 10, 2023 (WO) PCT/CN2023/000034
Jul. 12, 2023 (WO) PCT/CN2023/106924

(51) Int. Cl. C12N 15/113

(52) U.S. Cl. CPC C12N 15/1131; C12N 15/113; C12N 15/1137; C12Y 207/11001; C12N 2310/11; C12N 2310/314; C12N 2310/315; C12N 2310/321; C12N 2310/322; C12N 2310/3231; C12N 2310/341

(57) ABSTRACT

Provided are modified multi-segmented antisense oligonucleotides and methods for decreasing target mRNA, DNA and protein expression. Such methods, oligonucleotides, and compositions are useful to treat, prevent, or ameliorate diseases or disorders.

### RELATED APPLICATIONS

This application claims the priority and benefit of PCT Application No. PCT/CN2023/000034, filed on Jan. 10, 2023, and PCT Application No. PCT/CN2023/106924, filed Jul. 12, 2023, the contents of each of which are incorporated herein in their entirety.

### BACKGROUND

Antisense technology is emerging as an effective means for reducing the expression of specific gene products and may therefore prove to be uniquely useful in a number of therapies. Antisense therapy differs from nucleoside therapy in that it can directly target the transcripts for the targets and antigens of interest and therapeutic value. Antisense technology is emerging as an effective means for reducing the expression of certain gene products and may therefore prove to be uniquely useful in a number of therapeutic, diagnostic, and research applications for the modulation of diseases of interest.

Many of the antisense oligonucleotides (ASOs) in current clinical use and testing have minimal efficacy in reducing target levels and/or caused safety concerns. Thus there exists a need in the art for improved target antisense oligonucleotides. Provided herein are compositions and methods that address the same.

### SUMMARY

The present disclosure provides modified multi-segmented antisense oligonucleotide structures that are complementary to a nucleotide sequence in a gene transcript. The modified multi-segmented antisense oligonucleotides of the disclosure comprise from 5′ to 3′ order, at least a 5′ wing segment; a first gap segment (G1); a first separator segment (S1); a second gap segment (G2); and a 3′ wing segment (W2), wherein each segment is joined by an internucleoside linkage; and wherein at least one nucleoside of each of W1, W2, and S1 is modified. In some embodiments, the modified multi-segmented antisense oligonucleotide may have additional separator segments, flanked by additional gap segments.

These modified multi-segmented antisense oligonucleotide contain separated gap regions in conjunction with strategically positioned binding enhancers. The target of these modified multi-segmented antisense oligonucleotides can be any target, for example a mammalian target or a viral target. In some aspects, the modified multi-segmented antisense oligonucleotides target HBV, DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C-III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) or hydroxy acid oxidase 1. In some embodiments, the modified multi-segmented antisense oligonucleotides target Matla, (pro)renin receptor/(P)RR, COVID-19 5′UTR, Acc1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 150 Dystrophin, Exon 151 Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, H1N1, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2a, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF1, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, or YAP1. They are useful for treating both acute and chronic disease conditions, including but not limited to wherein the target of the oligonucleotide is an oncogenic target, an inflammatory target, a metabolic disease target, a cardiovascular target, a liver disease target, a infectious disease target, a neurological disease target, a neuromuscular disease target, an eye disease target, a kidney disease target, a respiratory disease target, a blood disease target, a wound healing target, a transplantation target, an autoimmune disease target, a neuropsychiatric target, or the like.

The present disclosure is based, at least in part, on the finding that a discontinuous gap segment flanked between a 5′ wing segment and a 3′ wing segment, in a modified multi-segmented antisense oligonucleotides of the disclosure, provides improved activity over conventional gapmers (antisense oligonucleotides composed of a central DNA segment flanked by wing segments). One or more separator segments placed directly in between two gap segments, thus rendering the discontinuity of the gap segment, surprisingly provides an improved activity of the modified multi-segmented antisense oligonucleotide. The inventors have unexpectedly discovered that the placement of separator segments at specific positions of the multi-segmented antisense oligonucleotides of the disclosure provides improved activity. Without being bound by theory or mechanisms, the one or more separator segments flanked between gap segments provides an advantageous secondary structure for improved physicochemical properties (e.g. solubility) and alternative interactions with nucleases (e.g. endonucleases such as RNase H) when duplexed with a target nucleic acid, thereby increasing the ability of the modified multi-segmented antisense oligonucleotides of the disclosure to target and/or degrade the target mRNA.

Further, specific types of nucleoside modifications in the separator segments provide unexpected improvements in activity. Without being bound by theory or mechanism, the inventors have observed that certain modifications of nucleosides in the separator segments, including 2′-O-methoxyethyl and 2′-O-methyl modifications, increase the ability of the modified multi-segmented antisense oligonucleotides of the disclosure to target and/or degrade the target mRNA.

Without being bound by theory or mechanism, certain combinations of nucleoside modifications and positioning of the separator segment(s) within the modified multi-segmented antisense oligonucleotide, improve interactions with RNase H endonuclease, without compromising hybridization to the target sequence.

A substitution of a nucleoside with a sugar-modified nucleoside (e.g. 2′-O-MOE) in conventional gapmers (i.e. those that are not multi-segmented) results in enhanced binding affinity with a target sequence. This type of enhancement has been explored in conventional gapmer designs to form more stable duplexes with a target sequence, thereby improving the gapmer activity. The stability of the duplexes can be determined by quantifying the melting temperature (Tm). A higher melting temperature is conventionally indicative of enhanced binding with a target sequence and would be expected to result in an increased recruitment of nucleases and functional activity (e.g. silencing of target expression).

Distinct from the current teachings in the art and distinct from conventional gapmers that are not multi-segmented, the inventors have unexpectedly discovered that introducing certain nucleoside (e.g. 2′-O-MOE) as a separator at certain locations in a continuous sequence of deoxynucleosides unexpectedly reduced Tm, instead of increasing it. More surprisingly, the modified multi-segmented antisense oligonucleotides with such Tm reduction correlated with improved biological activities and potency compared to the corresponding conventional gapmer designs. Without being bound by theory or mechanism, the certain combination of nucleoside modifications and positioning of the separator segment(s) within the full length multi-segmented antisense oligonucleotide may result in a change in secondary structure, which, in turn, is advantageous for enhanced interaction with nucleases (e.g. endonucleases such as RNase H) and improved functional activity.

The present disclosure is based, at least in part, on the finding that certain nucleoside modifications at certain positions of the 5′ wing segment and/or 3′ wing segment decrease in vivo toxicity. The inventors have unexpectedly discovered certain modified nucleosides at certain positions in the 3′ wing segment (e.g. LNA) decreased in vivo toxicity. Without being bound by theory or mechanism, certain combinations of nucleoside modifications and positioning within the 5′ wing segment and/or the 3′ wing segment, contributes to the complementarity of the modified multi-segmented antisense oligonucleotide while reducing interactions with off-target sequences. The modified multi-segmented antisense oligonucleotides described herein have one or more of increased in vivo activity, reduced off-target binding, reduced toxicity, and wider safety margin, which are advantageous for therapeutic use.

Provided herein are methods, modified multi-segmented antisense oligonucleotides and compositions for modulating expression of target mRNA and protein.

Also provided are methods, modified multi-segmented antisense oligonucleotides, and compositions useful for preventing, treating, and ameliorating diseases, disorders, and conditions.

### BRIEF DESCRIPTION OF THE DRAWINGS

FIGS. 1A-1E show a series of graphs depicting the inhibition of PCSK9 mRNA levels in Hepa1-6 cells following the treatment with modified multi-segmented antisense oligonucleotides of the disclosure.

FIG. 2A-2F show a series of graphs depicting the inhibition of PCSK9 mRNA levels in Hepa1-6 cells following the treatment with modified multi-segmented antisense oligonucleotides of the disclosure.

FIG. 3A-3E show a series of graphs depicting the inhibition of DMPK mRNA levels in Sol8 cells following the treatment with modified multi-segmented antisense oligonucleotides of the disclosure.

FIG. 4A-4F show a series of graphs depicting the inhibition of DMPK mRNA levels in Sol8 cells following the treatment with modified multi-segmented antisense oligonucleotides of the disclosure.

FIG. 5A-5D show a series of graphs depicting the inhibition of TTR mRNA levels in AML-12 cells following the treatment with modified multi-segmented antisense oligonucleotides of the disclosure.

FIG. 6A-6D show a series of graphs depicting the inhibition of TTR mRNA levels in AML-12 cells following the treatment with modified multi-segmented antisense oligonucleotides of the disclosure.

FIG. 7A-7D show a series of graphs depicting the inhibition of AAT mRNA levels in AML-12 cells following the treatment with modified multi-segmented antisense oligonucleotides of the disclosure.

FIG. 8 shows a graph depicting the effect of modified multi-segmented antisense oligonucleotides of the disclosure on plasma PCSK9 protein levels.

FIG. 9 shows a graph depicting the percent inhibition of plasma PCSK9 protein levels following treatment with modified multi-segmented antisense oligonucleotides of the disclosure.

FIG. 10 shows a graph depicting the effect of modified multi-segmented antisense oligonucleotides of the disclosure on plasma TTR protein levels.

FIG. 11 shows a graph depicting the percent inhibition of plasma TTR protein levels following treatment with modified multi-segmented antisense oligonucleotides of the disclosure.

FIGS. 12A-12B show graphs depicting the effect of modified multi-segmented antisense oligonucleotides of the disclosure on hepatitis B virus (HBV) surface antigen (HBsAg) levels in genotype C HBV transgenic mice.

FIG. 13 shows a graph depicting the effect of modified multi-segmented antisense oligonucleotides of the disclosure on HBsAg levels in pcDNA3.1-preS2-GTD HDI-HBV mice.

FIG. 14 shows a graph depicting the effect of modified multi-segmented antisense oligonucleotides of the disclosure on HBsAg levels in pcDNA3.1-preS2-GTD HDI-HBV mice.

FIG. 15 shows a graph depicting the effect of modified multi-segmented antisense oligonucleotides of the disclosure on HBsAg levels in pcDNA3.1-preS2-GTD HDI-HBV mice.

FIGS. 16A-16B shows a graph depicting the effect of modified multi-segmented antisense oligonucleotides of the disclosure on HBsAg levels in pcDNA3.1-preS2-GTD HDI-HBV mice.

FIG. 17 shows a graph depicting the effect of modified multi-segmented antisense oligonucleotides of the disclosure on HBsAg levels in pcDNA3.1-preS2-GTD HDI-HBV mice.

FIG. 18 shows a graph depicting the effect of modified multi-segmented antisense oligonucleotides of the disclosure on HBsAg levels in in pAAV-1.2HBV-GTA HDI-HBV mice.

FIG. 19 shows a graph depicting the effect of modified multi-segmented antisense oligonucleotides of the disclosure on HBsAg levels in pcDNA3.1-preS2-GTD HDI-HBV mice.

FIG. 20 shows a 20% Precast-UREA-TBE PAGE gel of RNase H cleavage products. Lane 1: AUS1233, Lane 2: AUS1493, Lane 3: AUS1493, Lane 4: AUS1013, Lane 5: AUS1014, Lane 6: AUS1015, Lane 7: AUS1212, Lane 8: AUS1654, Lane 9: AUS1655, Lane 10: Molecular weight markers

FIG. 21 shows a table of exemplary modified multi-segmented antisense oligonucleotides of the disclosure and are incorporated into the detailed description in their entirety. The modifications at each position of the modified multi-segmented antisense oligonucleotide sequences are read using the legend in Table 2.2. AUS1010 to AUS1714 (SEQ ID NO: 11 to SEQ ID NO: 666) contain examples of the modified multi-segmented antisense oligonucleotides of the disclosure.

FIG. 22 shows a table of exemplary modified multi-segmented antisense oligonucleotides of the disclosure. In FIG. 22, lowercase font represents a 2′-deoxynucleoside, and uppercase font represents a nucleoside with any one of the modifications shown in the section titled Exemplary Modifications, such as a 2′-O-methoxyethyl nucleoside, 2′-O-methoxyethyl cytidine, 2′-O-methyl nucleoside, 2′-O-methyl nucleoside, 2′-OH nucleoside, GNA, LNA, LNA with cytosine base, 2′-Fluoro 2′-deoxy nucleoside, 2′-Fluoro 2′-deoxy cytidine, 2′-F-Arabinonucleic Acid (2′-F-ANA), or 2′-F-Arabinonucleic Acid (2′-F-ANA), or a combination thereof. In FIG. 22, lowercase “c” represents a 2′-deoxy cytidine or 2′-deoxy 5-methylcytidine, and upper case “C” represents a cytidine or 5-methylcytidine with or without the above described sugar modifications. SEQ ID NOS: 5,000 to SEQ ID NO: 30,983 are exemplary modified multi-segmented antisense oligonucleotides.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxyethyl modification at the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“3′ target site” refers to the region of a target nucleic acid which is complementary to the 3′-end of a particular antisense oligonucleotide.

“5′ target site” refers to the region of a target nucleic acid which is complementary to the 5′-end of a particular antisense oligonucleotide.

“5-methylcytosine” means a cytosine modified with a methyl group attached to the 5 position. A 5-methylcytosine is a modified nucleobase.

“About” means within ±7% of a value. For example, if it is stated, “the oligonucleotides affected at least about 70% inhibition of target”, it is implied that the target levels are inhibited within a range of 63% and 77%.

“Acceptable safety profile” means a pattern of side effects that is within clinically acceptable limits.

“Active pharmaceutical agent” means the substance or substances in a pharmaceutical composition that provide a therapeutic benefit when administered to an individual.

“Active target region” means a target region to which one or more active antisense oligonucleotides is targeted. “Active antisense oligonucleotides” means antisense oligonucleotides that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Animal” refers to a human or non-human animal, including, but not limited to, mice, rats, rabbits, dogs, cats, pigs, primates, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense oligonucleotide to its target nucleic acid. In certain embodiments, antisense activity is a decrease in the amount or expression of a target nucleic acid or protein encoded by such target nucleic acid.

“Antisense oligonucleotide” or “ASO” means an oligomeric oligonucleotide that is capable of undergoing hybridization to a target nucleic acid through hydrogen bonding. Examples of antisense oligonucleotides include single-stranded oligonucleotides having a nucleobase sequence that permits hybridization to a corresponding region or segment of a target nucleic acid.

“Antisense inhibition” means reduction of target nucleic acid levels in the presence of an antisense oligonucleotide complementary to a target nucleic acid compared to target nucleic acid levels in the absence of the antisense oligonucleotide.

“Antisense mechanisms” are all those mechanisms involving hybridization of a oligonucleotide with target nucleic acid, wherein the outcome or effect of the hybridization is either target degradation or target occupancy with concomitant stalling of the cellular machinery involving, for example, transcription or splicing.

The “area under the curve” or “AUC” is the integral of the concentration of a drug in blood plasma as a function of time. The AUC can be determined for the totality of time for which data is available, for example until the drug is no longer detectable (AUC₀₋ₜ), the area under the curve from time 0 extrapolated to infinity (AUC_{0-¥}), or for a particular truncated window of time, for example 24 hours after administration (AUC₀₋₂₄).

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two non-geminal carbon atoms. A bicyclic sugar is a modified sugar.

“Body weight” refers to an animal's whole body weight, inclusive of all tissues including adipose tissue.

“Cap structure” or “terminal cap moiety” means chemical modifications, which have been incorporated at either terminus of an antisense compound.

“cEt” or “constrained ethyl” means a bicyclic sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Complementarity” means the capacity for pairing between nucleobases of a first nucleic acid and a second nucleic acid.

“Diluent” means an ingredient in a composition that lacks pharmacological activity, but is pharmaceutically necessary or desirable. For example, in drugs that are injected, the diluent may be a liquid, e.g. saline solution.

“Dosage unit” means a form in which a pharmaceutical agent is provided, e.g. pill, tablet, or other dosage unit known in the art.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Duration” means the period of time during which an activity or event continues. In certain embodiments, the duration of treatment is the period of time during which doses of a pharmaceutical agent are administered.

“Effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Efficacy” means the ability to produce a desired effect.

“Expression” includes all the functions by which a gene's coded information is converted into structures present and operating in a cell. Such structures include, but are not limited to the products of transcription and translation.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 60%, 70%, 80%, 90%, 92%, 95%, 98% or 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

“Fully complementary” or “100% complementary” means each nucleobase of a first nucleic acid has a complementary nucleobase in a second nucleic acid. In certain embodiments, a first nucleic acid is an antisense oligonucleotide and a target nucleic acid is a second nucleic acid.

“Fully modified motif” refers to an antisense oligonucleotide comprising a contiguous sequence of nucleosides wherein essentially each nucleoside is a sugar modified nucleoside having uniform modification.

“Gapmer” means an antisense oligonucleotide in which an internal region having continuous sequence of linked deoxynucleosides that support recruitment of nuclease (e.g., a RNase, e.g. RNase H) positioned between external regions having one or more nucleosides, wherein the nucleosides comprising the internal region may be chemically distinct from the nucleoside or nucleosides comprising the external regions. The internal region may be referred to as the “gap” and each of the 5′ and 3′ external regions may be referred to as a “wing.”

A “gap” is an internal segment of a modified multi-segmented antisense oligonucleotide or of a gapmer that comprises one or more linked deoxynucleosides, and is positioned directly or indirectly between a 5′ wing (W1) and a 3′ wing (W2). A gap may be interchangeably referred to as a “gap” a “gap region” or a “gap segment”.

“Hybridization” means the annealing of complementary nucleic acid molecules. In certain embodiments, complementary nucleic acid molecules include, but are not limited to, an antisense oligonucleotide and a nucleic acid target. In certain embodiments, complementary nucleic acid molecules include, but are not limited to, an antisense oligonucleotide and a nucleic acid target.

“Immediately adjacent” means there are no intervening elements between the immediately adjacent elements.

“Individual” means a human or non-human animal selected for treatment or therapy.

“Individual compliance” means adherence to a recommended or prescribed therapy by an individual.

“Induce”, “inhibit”, “potentiate”, “elevate”, “increase”, “decrease” or the like, generally denote quantitative differences between two states. Such terms may refer to a statistically significant difference between the two states. Such terms are applied to, for example, levels of expression, and levels of activity. The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

“Inhibiting the expression or activity” refers to a reduction, blockade of the expression or activity and does not necessarily indicate a total elimination of expression or activity.

“Injection site reaction” means inflammation or abnormal redness of skin at a site of injection in an individual.

“Intraperitoneal administration” means administration through infusion or injection into the peritoneum.

“Intravenous administration” means administration into a vein.

“Lengthened” antisense oligonucleotides are those that have one or more additional

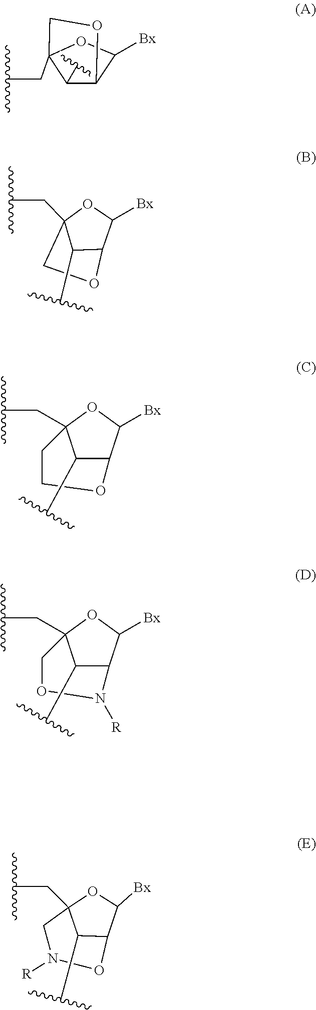

nucleosides relative to an antisense oligonucleotide disclosed herein.

“Locked nucleic acid” or “LNA” or “LNA nucleosides” means nucleic acid monomers having a bridge connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA; (B) β-D-Methyleneoxy (4′-CH₂—O-2′)-LNA; (C) Ethyleneoxy (4′-(CH₂)₂—O-2′) LNA; (D) Aminooxy (4′-CH₂—O—NI-2′) LNA; and (E) Oxyamino (4′-CH₂—NI—O-2′) LNA; as depicted below.

As used herein, LNA oligonucleotides include, but are not limited to, oligonucleotides having at least one bridge between the 4′ and the 2′ position of the sugar wherein each of the bridges independently comprises 1 or from 2 to 4 linked groups independently selected from —[C(R¹)(R²)]n-, —C(R¹)═C(R²)—, —C(R¹)═N—, —C(═NR¹)—, —C(═O)—, —C(═S)—, —O—, —Si(R′)₂—, —S(═O)ₓ— and —N(R¹)—; wherein: x is 0, 1, or 2; n is 1, 2, 3, or 4; each R¹ and R² is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ¹, NJ¹J², SJ¹, N₃, COOJ¹, acyl (C(═O)—H), substituted acyl, CN, sulfonyl (S(═O)₂-J¹), or sulfoxyl (S(═O)-J¹); and each J¹ and J² is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R¹)(R²)]ₙ—, —[C(R¹)(R²)]ₙ—O—, —C(R¹)(R²)—N(R¹)—O— or C(R¹)(R²)—O—N(R¹)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R¹)-2′ and 4′-CH₂—N(R¹)—O-2′- bridges, wherein each R¹ and R² is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. A-L-methyleneoxy (4′-CH₂—O-2′), an isomer of methyleneoxy (4′-CH₂—O-2′) LNA is also encompassed within the definition of LNA, as used herein.

A “maximum blood plasma concentration” or “Cₘₐₓ” refers to the highest concentration of a drug in the blood plasma after dose of the drug is given to a subject. Methods of measuring the concentration of drugs will be known to persons of ordinary skill in the art, and include, inter alia, liquid chromatography, and tandem mass spectrometry.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Monomer” refers to a single unit of an oligomer. Monomers include, but are not limited to, nucleosides and nucleotides, whether naturally occurring or modified.

“Motif” means the pattern of unmodified and modified nucleosides in an antisense oligonucleotide. “Natural sugar moiety” means a sugar moiety found in DNA (2′-H) or RNA (2′-OH). “Naturally occurring internucleoside linkage” means a 3′ to 5′ phosphodiester linkage.

A “modified multi-segmented antisense oligonucleotide” means an antisense oligonucleotide with at least five structural segments, comprising a 5′ wing (W1), a 3′ wing (W2), at least two gaps (at least G1 and G2) and at least one separator (at least an S1). The separator is flanked by two gap regions. The wings are at the terminal ends of the modified multi-segmented antisense oligonucleotide. From 5′ to 3′, an exemplary modified multi-segmented antisense oligonucleotide has the formula of Formula I, or Formula II. A modified multi-segmented antisense oligonucleotide of the disclosure further comprises at least one modified nucleoside in the 5′ wing, at least one nucleoside modification in the separator, and at least one modified nucleoside in the 3′ wing. A “modified multi-segmented antisense oligonucleotide” may be interchangeably referred to as a “multi-segmented modified antisense oligonucleotide” or “multi-segmented oligonucleotide”.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a nitrogenous heterocyclic base moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense oligonucleotide that is capable of base pairing with a nucleobase of its target nucleic acid. For example, if a nucleobase at a certain position of an antisense oligonucleotide is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Off-target effect” refers to an unwanted or deleterious biological effect associated with modulation of RNA or protein expression of a gene other than the intended target nucleic acid.

“Oligomeric compound” means a polymer of linked monomeric subunits which is capable of hybridizing to at least a region of a nucleic acid molecule.

“Oligonucleotide” means a polymer of linked nucleosides, through internucleoside linkages, each of the linked nucleosides can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, “peptide” refers to polypeptides and proteins.

“Pharmaceutically acceptable carrier” means a medium or diluent that does not interfere with the structure of the oligonucleotide. Certain such carriers enable pharmaceutical compositions to be formulated as, for example, tablets, pills, dragees, capsules, liquids, gels, syrups, slurries, suspension and lozenges for the oral ingestion by a subject.

“Pharmaceutically acceptable derivative” encompasses pharmaceutically acceptable salts, conjugates, prodrugs or isomers of the oligonucleotides described herein.

“Pharmaceutically acceptable salts” means physiologically and pharmaceutically acceptable salts of antisense oligonucleotides, i.e., salts that retain the desired biological activity of the parent oligonucleotide and do not impart undesired toxicological effects thereto.

“Pharmaceutical agent” means a substance that provides a therapeutic benefit when administered to an individual.

“Pharmaceutical composition” means a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Prevention” or “preventing” refers to delaying or forestalling the onset or development of a condition or disease for a period of time from hours to days, preferably weeks to months.

“Prodrug” means a therapeutic agent that is prepared in an inactive form that is converted to an active form (i.e., drug) within the body or cells thereof by the action of endogenous enzymes or other chemicals and/or conditions.

“Prophylactically effective amount” refers to an amount of a pharmaceutical agent that provides a prophylactic or preventative benefit to an animal.

“Recommended therapy” means a therapeutic regimen recommended by a medical professional for the treatment, amelioration, or prevention of a disease.

“Ribonucleotide” means a nucleotide having a hydroxy group at the 2′ position of the sugar portion of the nucleotide. Ribonucleotides may be modified with any of a variety of substituents.

“Salts” mean a physiologically and pharmaceutically acceptable salts of antisense oligonucleotides, i.e., salts that retain the desired biological activity of the parent oligonucleotide and do not impart undesired toxicological effects thereto.

“Segments” may be interchangeably referred to as “regions” or “portions”.

A “separator” in the modified multi-segmented antisense oligonucleotides of the disclosure is positioned directly in between, and separates two gap regions and is positioned in between two gap regions in a modified multi-segmented antisense oligonucleotide. The separator may have one or more nucleosides, wherein the nucleosides are chemically distinct from the nucleosides comprising the gap. A separator segment comprises nucleosides modified to impart properties such as enhanced inhibitory activity, increased binding affinity for a target nucleic acid, lowered in vivo toxicity or resistance to degradation by in vivo nucleases. A modified multi-segmented antisense oligonucleotide may comprise one or more separator segments. Exemplary modified multi-segmented antisense oligonucleotides of the disclosure may comprise one, two, three, four, five or six separator segments. In some embodiments, the modified multi-segmented antisense oligonucleotides comprise one separator segment. In some embodiments, the modified multi-segmented antisense oligonucleotides comprise two separator segments.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

“Significant,” as used herein means measurable or observable, e.g, a significant result, such as, a significant improvement or significant reduction generally refers to a measurable or observable result, such as a measurable or observable improvement or reduction.

“Sites,” as used herein, are defined as unique nucleobase positions within a target nucleic acid.

“Specifically hybridizable” refers to an antisense oligonucleotide having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments. “Stringent hybridization conditions” or “stringent conditions” refer to conditions under which an oligomeric oligonucleotide will hybridize to its target sequence, but to a minimal number of other sequences.

“Target nucleic acid,” “target RNA,” “target RNA transcript” and “nucleic acid target” all mean a nucleic acid capable of being targeted by antisense oligonucleotides.

“Target region” means a portion of a target nucleic acid to which one or more antisense oligonucleotides is targeted.

“Target segment” means the sequence of nucleotides of a target nucleic acid to which an antisense oligonucleotide is targeted.

“Therapeutically effective amount” means an amount of a pharmaceutical agent that provides a therapeutic benefit to an individual.

“Treatment” refers to administering a composition to effect an alteration or improvement of the disease or condition.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. (3-D-ribonucleosides) or a DNA nucleotide (i.e. (3-D-deoxyribonucleoside).

“Validated target segment” is defined as at least an 8-nucleobase portion (i.e. 8 consecutive nucleobases) of a target region to which an active oligomeric oligonucleotide is targeted.

A “wing” is a terminal segment of a gapmer or a modified multi-segmented antisense oligonucleotide, modified to impart to an oligonucleotide, properties such as enhanced inhibitory activity, enhanced biological activity, increased binding affinity for a target nucleic acid, lowered in vivo toxicity or resistance to degradation by in vivo nucleases. A wing may be referred to as a “wing” a “wing region” or a “wing segment”. As used herein, a wing comprises at least two linked nucleosides; a subset of which may comprise one or more deoxynucleosides, but the entire wing cannot be made up of only deoxynucleosides. In some embodiments, the wing comprises 2 to 8 linked nucleosides. In some embodiments, the wing comprises 2 to 6 linked nucleosides.

The modified multi-segmented antisense oligonucleotides of the disclosure comprise a 5′ wing segment (W1) located at the 5′ terminus of the modified multi-segmented antisense oligonucleotide and the residue at the 3′ end of the W1 is not a deoxynucleoside. The modified multi-segmented antisense oligonucleotides of the disclosure also comprise a 3′ wing segment (W2) located at the 3′ terminus of the modified multi-segmented antisense oligonucleotide and the 5′ terminal residue of the W2 is not a deoxynucleoside.

A 5′ wing (W1) begins at the 5′ terminus of the modified multi-segmented antisense oligonucleotide, extends in the 5′ to 3′ direction, and that is directly linked to a deoxynucleoside of a first gap, thus indicating the 3′ end of the W1 and the 5′ end of a first gap (G1).

A 3′ wing (W2) begins at the 3′ terminus of the modified multi-segmented antisense oligonucleotide, extends in the 3′ to 5′ direction, and that is directly linked to a deoxynucleoside of a gap, thus indicating the 3′ end of the last gap of and the 5′ end of the W2.

### Exemplary Modified Multi-Segmented Antisense Oligonucleotides

The disclosure provides for at least the following exemplary modified multi-segmented antisense oligonucleotides. The modified multi-segmented antisense oligonucleotides of the disclosure are contemplated to be complementary to the transcript of a gene, where, in some embodiments, the gene may be one or more of HBV, DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) or hydroxy acid oxidase 1.

In some embodiments, the modified multi-segmented antisense oligonucleotides of the disclosure may be complementary to the transcript of a gene, where the gene is one or more of Matla, (pro)renin receptor/(P)RR, COVID-19 5′UTR, Ace1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 150 Dystrophin, Exon 151 Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, H1N1, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2a, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF1, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, and YAP1.

In some embodiments, thus the modified multi-segmented antisense oligonucleotides provided herein have one or more of the ability to inhibit the expression of target genes, have higher biological activity, have low off-target binding and low toxicity, wider safety margin, which can be advantageous for therapeutic use. Further, in some embodiments, the modified multi-segmented antisense oligonucleotides of the disclosure have higher inhibition of target genes relative to a conventional gapmer (that is not multi-segmented) with the same template sequence.

Accordingly, in one aspect, the disclosure provides a modified multi-segmented antisense oligonucleotide comprising from 5′ to 3′:

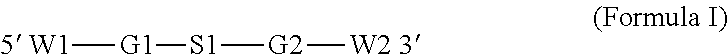

wherein:
W1 is a 5′ wing segment;
W2 is a 3′ wing segment;
G1 is a first gap segment;
S1 is a first separator segment;
G2 is a second gap segment;
- is an internucleoside linkage; and
at least one nucleoside of each of the W1, W2, and S1 is modified.

In some embodiments, the modified multi-segmented antisense oligonucleotide is of Formula I, and the W1 comprises 2-8 linked nucleosides, the G1 comprises 2-7 linked deoxynucleosides, the S1 comprises 1-2 nucleosides, the G2 comprises 2-7 linked deoxynucleosides and the W2 comprises 2-8 linked nucleosides.

In some embodiments, the modified multi-segmented antisense oligonucleotide is of Formula I, and the W1 comprises 2-8 linked nucleosides, the G1 comprises 2-7 linked deoxynucleosides, the S1 comprises 1 nucleoside, the G2 comprises 2-7 linked deoxynucleosides and the W2 comprises 2-8 linked nucleosides.

In some embodiments, the modified multi-segmented antisense oligonucleotide is of Formula I, and the W1 comprises 2-8 linked nucleosides, the G1 comprises 2-7 linked deoxynucleosides, the S1 comprises 2 nucleosides, the G2 comprises 2-7 linked deoxynucleosides and the W2 comprises 2-8 linked nucleosides.

In some embodiments, the modified multi-segmented antisense oligonucleotide is of Formula I, and the W1 comprises 2-6 linked nucleosides, the G1 comprises 2-7 linked deoxynucleosides, the S1 comprises 1 nucleoside, the G2 comprises 2-7 linked deoxynucleosides and the W2 comprises 2-6 linked nucleosides.

In some embodiments, the modified multi-segmented antisense oligonucleotide is of Formula I, and the W1 comprises 2-8 linked nucleosides, the G1 comprises 2-7 linked deoxynucleosides, the S1 comprises 1 nucleoside, the G2 comprises 2-7 linked deoxynucleosides and the W2 comprises 2-8 linked nucleosides.

In some embodiments, the modified multi-segmented antisense oligonucleotide is of Formula I, and W1 comprises 4-6 linked nucleosides, G1 comprises 1-6 linked deoxynucleosides, S1 comprises 1 linked nucleoside, G2 comprises 1-6 linked deoxynucleosides, and W2 comprises 4-6 linked nucleosides.

In some embodiments, the modified multi-segmented antisense oligonucleotide is of Formula I, and W1 comprises 4-6 linked nucleosides, G1 comprises 5 linked deoxynucleosides, S1 comprises 1 linked nucleoside, G2 comprises 5 linked deoxynucleosides, and W2 comprises 4-6 linked nucleosides.

In some embodiments, the modified multi-segmented antisense oligonucleotide is of Formula I, and W1 comprises 4 linked nucleosides, G1 comprises 5 linked deoxynucleosides, S1 comprises 1 linked nucleoside, G2 comprises 5 linked deoxynucleosides, and W2 comprises 5 linked nucleosides.

In some embodiments, the modified multi-segmented antisense oligonucleotide is of Formula I, and W1 comprises 5 linked nucleosides, G1 comprises 4 linked deoxynucleosides, S1 comprises 1 linked nucleoside, G2 comprises 5 linked deoxynucleosides, and W2 comprises 5 linked nucleosides.

In some embodiments, the modified multi-segmented antisense oligonucleotide is of Formula I, and the W1-G1-S1-G2-W2 motif is 2-4-1-5-2, 3-4-1-4-2, 3-4-1-5-3, 3-4-1-5-4, 3-4- 1-5-8, 3-5-1-4-2, 3-5-1-5-2, 3-5-1-5-3, 3-6-1-5-5, 3-6-1-6-4, 4-4-1-5-3, 4-4-1-5-4, 4-5-1-4-13, 4-5-1-4-6, 4-5-1-5-1, 4-5-1-5-10, 4-5-1-5-11, 4-5-1-5-13, 4-5-1-5-3, 4-5-1-5-4, 4-5-1-5-5, 4-5-1-5-6, 4-5-1-5-7, 4-5-1-5-8, 4-5-1-5-9, 4-5-1-6-4, 4-6-1-4-5, 5-1-1-8-5, 5-2-1-7-5, 5-3-1-6-5, 5-4-1-4-6, 5-4-1-5-25, 5-4-1-5-4, 5-4-1-5-5, 5-4-1-6-4, 5-4-1-7-3, 5-4-1-8-2, 5-4-2-4-5, 5-5-2-5-5, 5-5-2-6-5, 5-6-2-6-5, 5-5-1-4-25, 5-5-1-4-5, 5-6-1-3-5, 5-7-1-2-5, 5-8-1-1-5, 6-3-1-3-7, 6-3-1-4-6, 6-4-1-4-5 or 6-7-1-1-5, with each number representing the number of nucleosides. That is, for example a modified multi-segmented antisense oligonucleotide of Formula I (W1-G1-S1-G2-W2) that is 2-4-1-5-2 has 2 nucleosides in W1; 4 nucleosides in G1; 1 nucleoside in S1; 5 nucleosides in G2; and 2 nucleosides in W2.

In some embodiments, the modified multi-segmented antisense oligonucleotide is of Formula I, and the W1-G1-S1-G2-W2 motif is 4-5-1-5-5. In some embodiments, the modified multi-segmented antisense oligonucleotide is of Formula I, and the W1-G1-S1-G2-W2 motif is 5-4-1-5-5. In some embodiments, the modified multi-segmented antisense oligonucleotide is of Formula I, and the W1-G1-S1-G2-W2 motif is 5-5-1-5-5. In some embodiments, the modified multi-segmented antisense oligonucleotide is of Formula I, and the W1-G1-S1-G2-W2 motif is 5-5-1-4-5. In some embodiments, the modified multi-segmented antisense oligonucleotide is of Formula I, and the W1-G1-S1-G2-W2 motif is 6-5-1-5-5.

In some embodiments, the modified multi-segmented antisense oligonucleotide comprises from 5′ to 3′:

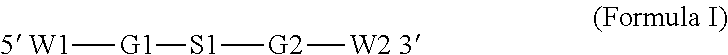

wherein:
S2 is a second separator segment;
G3 is a third gap segment;
- is an internucleoside linkage; and
at least one nucleoside of each of W1, S1, S2, and W2 is a modified nucleoside.

In some embodiments, the modified multi-segmented antisense oligonucleotide is of Formula II, and the W1 comprises 2-8 linked nucleosides, the G1 comprises 2-7 linked deoxynucleosides, the S1 comprises 1 linked nucleoside, the G2 comprises 1-2 linked deoxynucleosides, S2 comprises 1 linked nucleoside, the G3 comprises 2-7 linked deoxynucleosides and the W2 comprises 2-8 linked nucleosides.

In some embodiments, the modified multi-segmented antisense oligonucleotide is of Formula II, and the W1 comprises 2-8 linked nucleosides, the G1 comprises 2-7 linked deoxynucleosides, the S1 comprises 1 linked nucleoside, the G2 comprises 1 linked deoxynucleoside, S2 comprises 1 linked nucleoside, the G3 comprises 2-7 linked deoxynucleosides and the W2 comprises 2-8 linked nucleosides.

In some embodiments, the modified multi-segmented antisense oligonucleotide is of Formula II, and the W1 comprises 2-8 linked nucleosides, the G1 comprises 2-7 linked deoxynucleosides, the S1 comprises 1 linked nucleoside, the G2 comprises 2 linked deoxynucleosides, S2 comprises 1 linked nucleoside, the G3 comprises 2-7 linked deoxynucleosides and the W2 comprises 2-8 linked nucleosides.

In some embodiments, the modified multi-segmented antisense oligonucleotide is of Formula II, and the W1 comprises 2-6 linked nucleosides, the G1 comprises 2-7 linked deoxynucleosides, the S1 comprises 1 linked nucleoside, the G2 comprises 1-2 linked deoxynucleosides, S2 comprises 1 linked nucleoside, the G3 comprises 2-7 linked deoxynucleosides and the W2 comprises 2-6 linked nucleosides.

In some embodiments, the modified multi-segmented antisense oligonucleotide is of Formula II, and the W1 comprises 2-6 linked nucleosides, the G1 comprises 2-7 linked deoxynucleosides, the S1 comprises 1 linked nucleoside, the G2 comprises 1 linked deoxynucleoside, S2 comprises 1 linked nucleoside, the G3 comprises 2-7 linked deoxynucleosides and the W2 comprises 2-6 linked nucleosides.

In some embodiments, the modified multi-segmented antisense oligonucleotide is of Formula II, and the W1 comprises 2-6 linked nucleosides, the G1 comprises 2-7 linked deoxynucleosides, the S1 comprises 1 linked nucleoside, the G2 comprises 2 linked deoxynucleosides, S2 comprises 1 linked nucleoside, the G3 comprises 2-7 linked deoxynucleosides and the W2 comprises 2-6 linked nucleosides.

In some embodiments, the modified multi-segmented antisense oligonucleotide is of Formula I, and the W1-G1-S1-G2-S2-G3-W2 motif is 5-1-1-3-1-3-6, 5-1-1-3-1-4-5, 5-1-1-6-1-1-5, 5-2-1-2-1-2-7, 5-2-1-2-1-4-5, 5-2-1-4-1-2-5, 5-3-1-1-1-4-5, 5-3- 1-2-1-3-5, 5-3-1-4-1-1-5, 5-4-1-1-1-3-5, 5-4-1-2-1-1-6, 5-4-1-2-1-2-5, 5-4-1-3-1-1-5, 6-3-1-1-1-1-7, 6-3-1-3-1-1-5, 6-4-1-1-1-2-5 or 7-2-1-2-1-2-5. That is, for example a modified multi-segmented antisense oligonucleotide of Formula II (W1-G1-S1-G2-S2-G3-W2) that is 5-1-1-3-1-3-6 has 5 nucleosides in W1; 1 nucleoside in G1; 1 nucleoside in S1; 3 nucleosides in G2; 1 nucleoside in S1; 3 nucleosides in G2; 1 nucleoside in S2; 3 nucleosides in G3; and 2 nucleosides in W2.

In some embodiments, the modified multi-segmented antisense oligonucleotide comprises from 5′ to 3′:

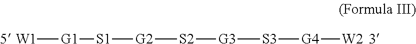

wherein:
S3 is a third separator segment;
G4 is a fourth gap segment; and
- is an internucleoside linkage; and at least one nucleoside of each of W1, S1, S2, S3 and W2 is a modified nucleoside.

In some embodiments, the modified multi-segmented antisense oligonucleotide comprises from 5′ to 3′:

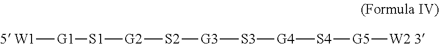

wherein:
S4 is a fourth separator segment;
G5 is a fifth gap segment; and
- is an internucleoside linkage; and at least one nucleoside of each of W1, S1, S2, S3, S4 and W2 is a modified nucleoside. In some embodiments, the modified multi-segmented antisense oligonucleotide comprises from 5′ to 3′:

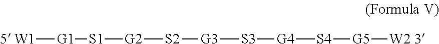

wherein:
S5 is a fifth separator segment;
G6 is a sixth gap segment; and
- is an internucleoside linkage; and at least one nucleoside of each of W1, S1, S2, S3, S4, S5, and W2 is a modified nucleoside.

In some embodiments, the modified multi-segmented antisense oligonucleotide comprises from 5′ to 3′:

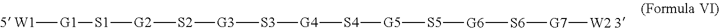

wherein:
S6 is a sixth separator segment;
G7 is a seventh gap segment; and
- is an internucleoside linkage; and at least one nucleoside of each of W1, S1, S2, S3, S4, S5, S6, and W2 is a modified nucleoside.

In some embodiments, W1 comprises 2 to 25 linked nucleosides. In some embodiments, W1 comprises one or more linked deoxynucleosides.

In some embodiments, W2 comprises 2 to 35 linked nucleosides. In some embodiments, W2 comprises one or more linked deoxynucleosides.

In some embodiments, any one or more of G1, G2, G3, G4, G5, G6, and/or G7 comprises 1 to 10 linked deoxynucleosides.

In some embodiments, any one or more of S1, S2, S3, S4, S5, and/or S6 comprise 1, 2, 3, 4, or 5 linked nucleosides.

In some embodiments, the modified multi-segmented antisense oligonucleotide is 12-50 nucleobases in length, 13-50 nucleobases in length, or 18-50 nucleobases in length. In some embodiments, the modified multi-segmented antisense oligonucleotide is about 12, 13, 14, 15, 16, 17, 18, 19, 20, 21, 22, 23, 24, 25, 26, 27, or 28 nucleobases in length. In some embodiments, the modified multi-segmented antisense oligonucleotide is at least 16 nucleobases in length. In some embodiments, the modified multi-segmented antisense oligonucleotide is at least 17 nucleobases in length. In some embodiments, the modified multi-segmented antisense oligonucleotide is at least 18 nucleobases in length. In some embodiments, the modified multi-segmented antisense oligonucleotide is at least 19 nucleobases in length. In some embodiments, the modified multi-segmented antisense oligonucleotide is 20 nucleobases in length.

In some embodiments, the modified multi-segmented antisense oligonucleotide is of Formula I, and the length of the G1-S1-G2 is between 6 and 14 nucleobases in length. In some embodiments, the modified multi-segmented antisense oligonucleotide is of Formula I, and the length of the G1-S1-G2 is between 8 and 12 nucleobases in length. In some embodiments, the modified multi-segmented antisense oligonucleotide is of Formula II, and the length of the G1-S1-G2-S2-G3 is between 8 and 13 nucleobases in length. In some embodiments, the modified multi-segmented antisense oligonucleotide is of Formula II, and the length of the G1-S1-G2-S2-G3 is between 8 and 12 nucleobases in length.

The following embodiments pertain to any of Formula I-VI: In some embodiments, G1, G2, G3, G4, G5, G6, and G7 can comprise a nucleoside comprising a modification to a 2′-deoxynucleoside. In some embodiments, any one or more of G1, G2, G3, G4, G5, G6, and G7 comprises a nucleoside comprising a 2′-deoxy 5-methylcytidine sugar modification. In some embodiments, S1, S2, S3, S4, S5, and/or S6 comprises a nucleoside comprising a 2′-O-methoxyethyl sugar modification. In some embodiments, S1, S2, S3, S4, S5, and/or S6 comprises a nucleoside comprising a 5-methylcytidine. In some embodiments, S1, S2, S3, S4, S5, and/or S6 comprises a nucleoside comprising a 2′-O-methyl sugar modification. In some embodiments, S1, S2, S3, S4, S5, and/or S6 comprises a nucleoside comprising a 2′-OH sugar modification. In some embodiments, S1, S2, S3, S4, S5, and/or S6 comprises a nucleoside comprising a 2′-fluoro sugar modification. In some embodiments, S1, S2, S3, S4, S5, and/or S6 comprises a nucleoside comprising a 2′-fluoro-arabinonucleic acid (2′-fluoro-ANA) sugar modification. In some embodiments, S1, S2, S3, S4, S5, and/or S6 comprises a glycol nucleic acid (GNA). In some embodiments, S1, S2, S3, S4, S5, and/or S6 comprises a bridged nucleic acid (e.g., LNA, cET, cMOE). In some embodiments, S1, S2, S3, S4, S5, and/or S6 comprises a LNA. In some embodiments, W1 comprises a nucleoside comprising a 2′-deoxy sugar modification. In some embodiments, W1 comprises a nucleoside comprising a 2′-O-methoxyethyl sugar modification. In some embodiments, W1 comprises a 2′-O-methoxyethyl 5-methylcytidine at position 2 of a sequence of a 20-mer (a sequence having 20 nucleobases). In some embodiments, W1 comprises a nucleoside comprising a 2′-O-methyl sugar modification. In some embodiments, W1 comprises a 2′-O-methyl 5-methylcytidine at position 2 of a 20-mer. In some embodiments, W1 comprises a nucleoside comprising a 2′-fluoro sugar modification. In some embodiments, W1 comprises a nucleoside comprising a 2′-fluoro-arabinonucleic acid (2′-fluoro-ANA) modification. In some embodiments, W1 comprises a glycol nucleic acid (GNA). In some embodiments, W1 comprises a modified nucleoside and wherein the modified nucleoside is a bridged nucleic acid (e.g., LNA, cET, cMOE). In some embodiments, W1 comprises a modified nucleoside and wherein the modified nucleoside is a locked nucleic acid (LNA). In some embodiments, W2 comprises a nucleoside comprising a 2′-deoxy sugar modification. In some embodiments, W2 comprises a nucleoside comprising a 2′-O-methoxyethyl sugar modification (e.g. wherein the 2′-O-methoxyethyl sugar modification is at positions 15, 16, 17, 18, 19 and/or 20 of a 20-mer). In some embodiments, W2 comprises a 2′-O-methoxyethyl 5-methylcytidine. In some embodiments, W2 comprises a nucleoside comprising a 2′-O-methyl sugar modification (e.g. wherein the 2′-O-methyl sugar modification is at positions 15, 16, 17, 18, 19 and/or 20 of a 20-mer. In some embodiments, W2 comprises a nucleoside comprising a 2′-fluoro sugar modification. In some embodiments, W2 comprises a nucleoside comprising a 2′-fluoro-arabinonucleic acid (2′-fluoro-ANA) modification. In some embodiments, W2 comprises a modified nucleoside and wherein the modified nucleoside is a glycol nucleic acid (GNA). In some embodiments, W2 comprises a modified nucleoside and wherein the modified nucleoside is a bridged nucleic acid (e.g, LNA, cET, cMOE). In some embodiments, W2 comprises a modified nucleoside and wherein the modified nucleoside is a locked nucleic acid (LNA) (e.g. wherein the LNA is at positions 16, 17, 18, 19 and/or 20 of a 20-mer).

### Exemplary Modifications

The modified multi-segmented antisense oligonucleotides of the disclosure may comprise linked nucleosides, e.g. linked deoxynucleosides, linked ribonucleosides, and linked deoxyribonucleosides. Accordingly, the modified multi-segmented antisense oligonucleotides of the disclosure may comprise deoxyribonucleotides, ribonucleotides, or a mixture of both. In some embodiments, the modified multi-segmented antisense oligonucleotide is a modified modified multi-segmented antisense oligonucleotide, e.g. the modified multi-segmented antisense oligonucleotide comprises one or more sugar modifications, one or more modified internucleoside linkages, and/or one or more base modifications. Features of modified multi-segmented antisense oligonucleotides of the disclosure are discussed in more detail below.

In some embodiments, the modified multi-segmented antisense oligonucleotide is conjugated to a moiety or a conjugate, referred to as conjugated modified multi-segmented antisense oligonucleotides. Features of conjugated modified multi-segmented antisense oligonucleotides of the disclosure are discussed in more detail below. Conjugated modified multi-segmented antisense oligonucleotides may be modified, or unmodified.

In some embodiments, an modified multi-segmented antisense oligonucleotide of the disclosure comprises the sequence of any one of SEQ ID NOS: 11-1453 or 5000-30983, or a modified multi-segmented antisense oligonucleotide comprising 1, 2, 3, 4, 5, 6, 7, 8, 9, 10, 11, 12, 13 or 14 sequence modifications thereto. The term “sequence modification” as used herein refers to the identity of the sequence (e.g. a modification from an A to a T).

In some embodiments, an modified multi-segmented antisense oligonucleotide of the disclosure comprises the sequence of any one of SEQ ID NOS: 11-1453 or 5000-30983, or an modified multi-segmented antisense oligonucleotide comprising at least 60%, 65%, 70%, 75%, 80%, 85%, 90%, 95% or 99% sequence identity thereto.

In some embodiments the vaccine comprises only one unique modified multi-segmented antisense oligonucleotide—for clarity, the vaccine may comprise copies of the same unique modified multi-segmented antisense oligonucleotide. In some embodiments the vaccine comprises a plurality of different modified multi-segmented antisense oligonucleotides, e.g. at least 2, at least 3, at least 4, at least 5, at least 6, at least 7, at least 8, at least 9, or at least 10 or more different modified multi-segmented antisense oligonucleotides of the disclosure.

### Features of Modified Multi-Segmented Antisense Oligonucleotides

As contemplated herein, modified multi-segmented antisense oligonucleotides encompass substitutions or changes to internucleoside linkages, sugar moieties, and nucleobases. Modified multi-segmented antisense oligonucleotides confer desirable properties such as, for example, enhanced cellular uptake, enhanced affinity for a nucleic acid target, increased stability in the presence of nucleases, or increased inhibitory activity. Chemically modified nucleosides may also be employed to increase the binding affinity for its target nucleic acid.

In some embodiments, a modified multi-segmented antisense oligonucleotide of the disclosure comprises one or more modified internucleoside linkages. In some embodiments, a modified multi-segmented antisense oligonucleotide of the disclosure comprises one or more modified internucleoside linkages, wherein the modified internucleoside linkage is a phosphorodiamidate linkage between morpholino nucleoside mimetics. In some embodiments, a modified multi-segmented antisense oligonucleotide of the disclosure comprises one or more modified internucleoside linkages, wherein the modified internucleoside linkage is a phosphorothioate internucleoside linkage. In some embodiments, the phosphorothioate modification comprises a mixture of Sp and Rp stereoisomers. In some embodiments, the phosphorothioate modification comprises a Sp stereoisomer. In some embodiments, the phosphorothioate modification comprises a Rp stereoisomer.

In some embodiments, a modified multi-segmented antisense oligonucleotide of the disclosure comprises one or more sugar modifications. In some embodiments, a modified multi-segmented antisense oligonucleotide of the disclosure comprises one or more linked nucleosides comprising a 2′ sugar modification (sugar modifications at the C2′ position). In exemplary embodiments, a modified multi-segmented antisense oligonucleotide of the disclosure comprises one or more modifications to a 2′-deoxynucleoside, e.g. comprises one or more nucleosides comprising a 2′-O-methyl modification, 2′-O-ethyl modification, 2′-O-(2-methoxyethyl) modification, 2′-fluoro modification, 2′-amino modification, 2′-O-propyl modification, 2′-O-butyl modification, 2′-O-cyclopropylmethyl modification, 2′-O-(2-hydroxyethyl) modification, 2′-O-[2-(methylamino)-2-oxyethyl]modification, 2′-O-2-[2-(N,N-dimethylamino)ethoxy]ethyl]modification, 2′-O-(2-dimethylaminoethyl) modification, 2′-O-(3-aminopropyl) modification, 2′-O-(2-aminopropyl) modification, 2′-O-(2-hydroxyisopropyl) modification, 2′-O-(2-methoxyisopropyl) modification, 2′-O-(2-dimethylaminoisopropyl) modification, 2′-O-(2-aminobutyl) modification, 2′-O-(2-hydroxybutyl) modification, 2′-O-(2-methoxybutyl) modification, 2′-O-(2-dimethylaminobutyl) modification, 2′-O-(2-aminocyclopropylmethyl) modification, 2′-O-(2-hydroxycyclopropylmethyl) modification, 2′-O-(2-methoxycyclopropylmethyl) modification, 2′-O-(2-dimethylaminocyclopropylmethyl) modification, 2′-O-[(2-guanidinium)ethyl]modification, and/or a 2′-fluoro-arabino (2′-fluoro-ANA) modification.

In some embodiments, a modified multi-segmented antisense oligonucleotide comprises one or more bicyclic sugar modified nucleosides. In some embodiments, a modified multi-segmented antisense oligonucleotide of the disclosure comprises one or more bridged nucleic acids wherein the 2′-oxygen is linked through bridging carbons to the 4′ carbon of the ribose to form a bridged nucleic acid (BNA), e.g. linked through a 2′,4′-methylene bridge to form a locked nucleic acid (LNA), or e.g. in 2′,4′-constrained 2′-O-ethyl modifications ((R)-cET or (S)-cET), or e.g. in 2′,4′-constrained 2′-O-methoxyethyl modifications ((R)-cMOE or (S)-cMOE).

In some embodiments, a modified multi-segmented antisense oligonucleotide comprises one or more carbocyclic LNA (cLNA) where the 2′-oxygen atom in LNA is replaced with a carbon atom. In some exemplary embodiments, a modified multi-segmented antisense oligonucleotide of the disclosure comprises one or more methanocarba bicyclo[3.1.0]hexane sugar either in the North C2′-exo (N-MC) or South C3′-exo forms (S-MC), or 2′-F-NMC. In some embodiments, a modified multi-segmented antisense oligonucleotide comprises (R)-Me-cLNA, or (S)-Me-cLNA, or F-LNA, or methylene-cLNA as shown below.

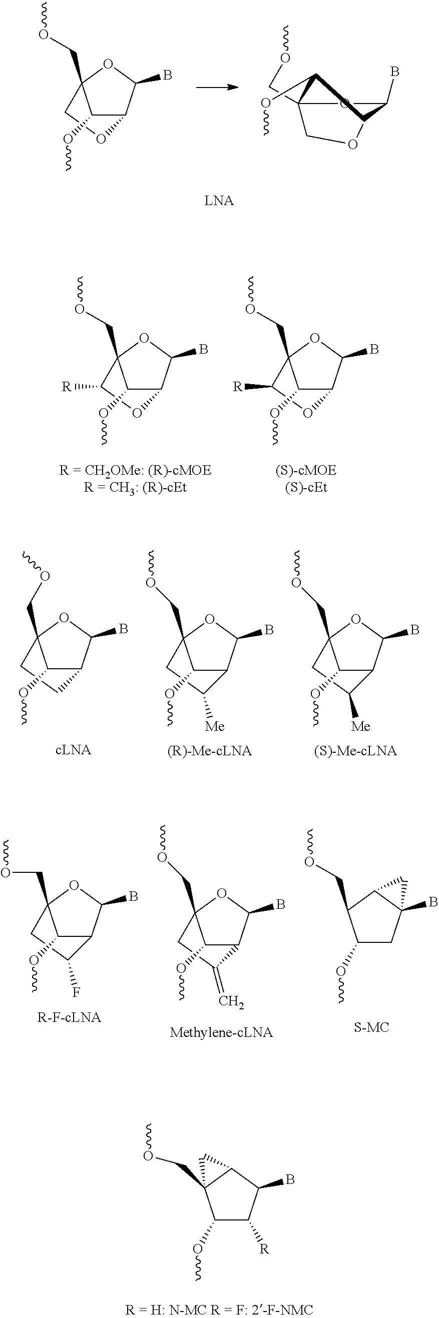

In some embodiments, a modified multi-segmented antisense oligonucleotide comprises one or more of an α-L-LNA, bcDNA, or tcDNA as shown below.

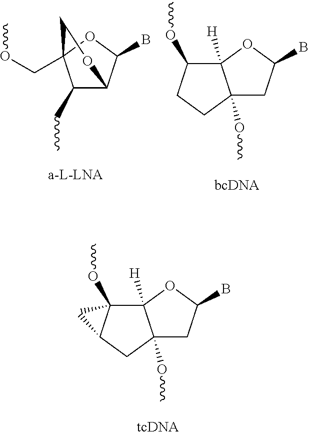

In some embodiments, a modified multi-segmented antisense oligonucleotide of the disclosure comprises one or more morpholino modifications wherein a morpholino group is in place of the ribose sugar. In some embodiments, a modified multi-segmented antisense oligonucleotide of the disclosure comprises phosphorodiamidate morpholino modification wherein the sugar-phosphate backbone is replaced with a phosphorodiamidate morpholino moiety. In some embodiments, a modified multi-segmented antisense oligonucleotide of the disclosure comprises one or more peptide nucleic acid (PNA) modification wherein the sugar-phosphate backbone or sugar-phosphorothioate backbone is replaced with a peptide backbone. In some embodiments, a modified multi-segmented antisense oligonucleotide of the disclosure comprises one or more glycol nucleic acid (GNA) modification, which sometimes is also referred to as glycerol nucleic acid, wherein a propylene glycol group is in place of the ribose sugar. In some embodiments, a modified multi-segmented antisense oligonucleotide of the disclosure comprises one or more phosphate analog E vinylphosphonate (E-VP), and the amide backbone linkage, as shown below.

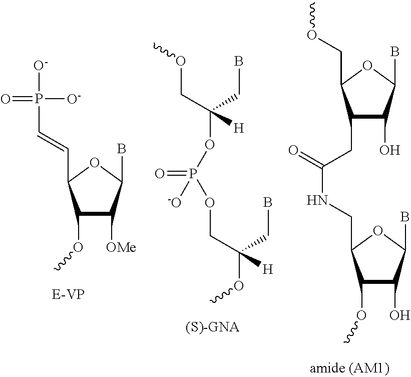

In some embodiments, a modified multi-segmented antisense oligonucleotide of the disclosure comprises one or more base modifications. In some embodiments, a modified multi-segmented antisense oligonucleotide of the disclosure comprises one or more nucleosides that are a 5-methylcytidine. In some embodiments, a modified multi-segmented antisense oligonucleotide of the disclosure comprises one or more nucleosides that are a N1-Methyl-pseudouridine.

In some embodiments, a modified multi-segmented antisense oligonucleotide of the disclosure comprises one or more modifications to the 3′-end and/or 5′-end of the oligonucleotides, e.g. 3′- and/or 5′-amino modifications.

In some embodiments, a modified multi-segmented antisense oligonucleotide of the disclosure comprises segmented oligonucleotides, creating one or more internal regions (gaps) having a plurality of nucleotides that supports RNaseH cleavage, which are positioned between external regions (5′ and 3′ wing segments) having a plurality of nucleotides that are chemically distinct from the nucleosides of the internal regions. In the case of an oligonucleotide exhibiting complementarity to a target having a segmented motif, the gap segment can hybridize with a target RNA sequence for endonuclease cleavage, while the wing segments comprise modified nucleosides. In some embodiments, the regions of a gaps are differentiated by the types of sugar moieties comprising each distinct region. The types of sugar moieties that are used to differentiate the regions of a gap may, in some embodiments, include β-D-ribonucleosides, β-D-deoxyribonucleosides, 2′-modified nucleosides (such 2′-modified nucleosides may include 2′-MOE and 2′-O—CH₃, among others as described in the preceding paragraphs, and bicyclic sugar modified nucleosides (such bicyclic sugar modified nucleosides may include those having a constrained ethyl). In some embodiments, nucleosides in the wings may include modified sugar moieties, including, for example 2′-MOE and bicyclic sugar moieties such as constrained ethyl or LNA. In some embodiments, the wings may include several modified and unmodified sugar moieties as described in the preceding paragraphs. In some embodiments, wings may include various combinations of 2′-MOE nucleosides, bicyclic sugar moieties such as constrained ethyl nucleosides or LNA nucleosides, and 2′-deoxynucleosides. There can be a single internal region (gap), or can be further segmented with separator segments, thus resulting in a discontinuous gap segments, which, in some embodiments provides improved activity over conventional oligonucleotides that have a continuous gap (e.g., reduction of HBsAg levels, HBeAg levels, and/or HBcAg levels in the serum or other body fluid or tissue). One or more separator segments placed directly in between gap segments can be beneficial

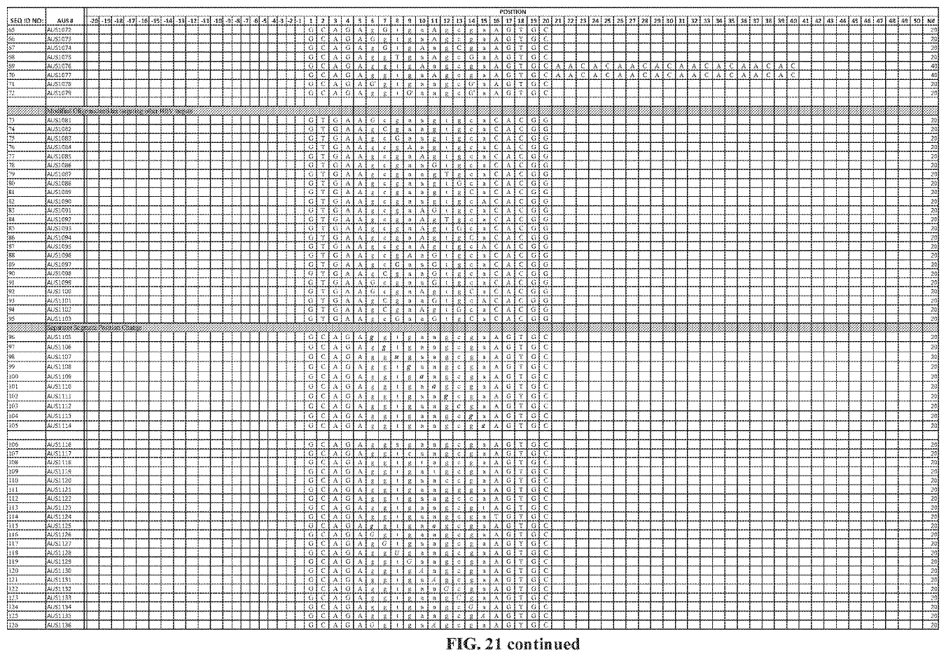

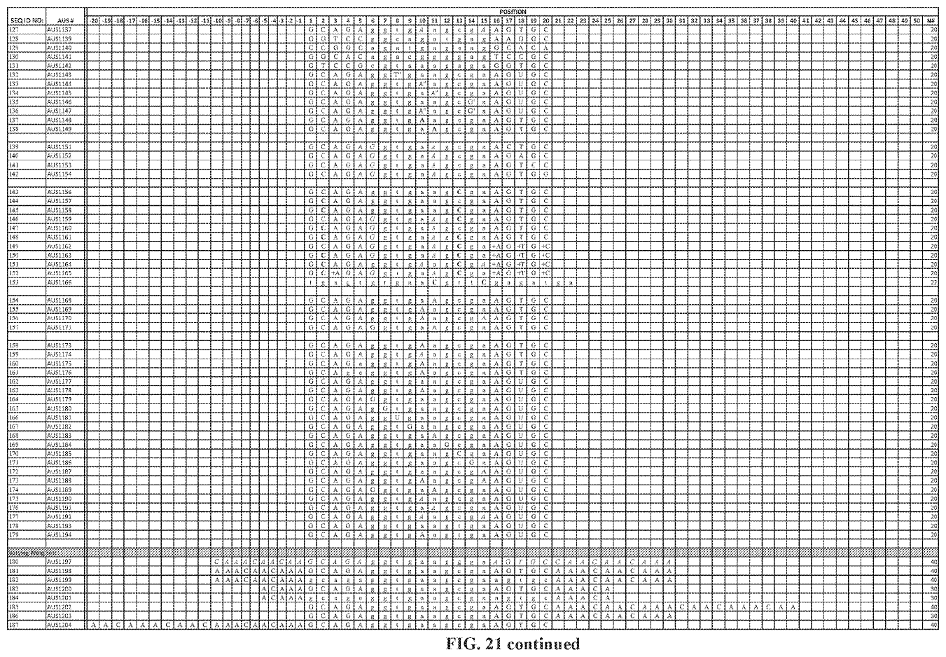

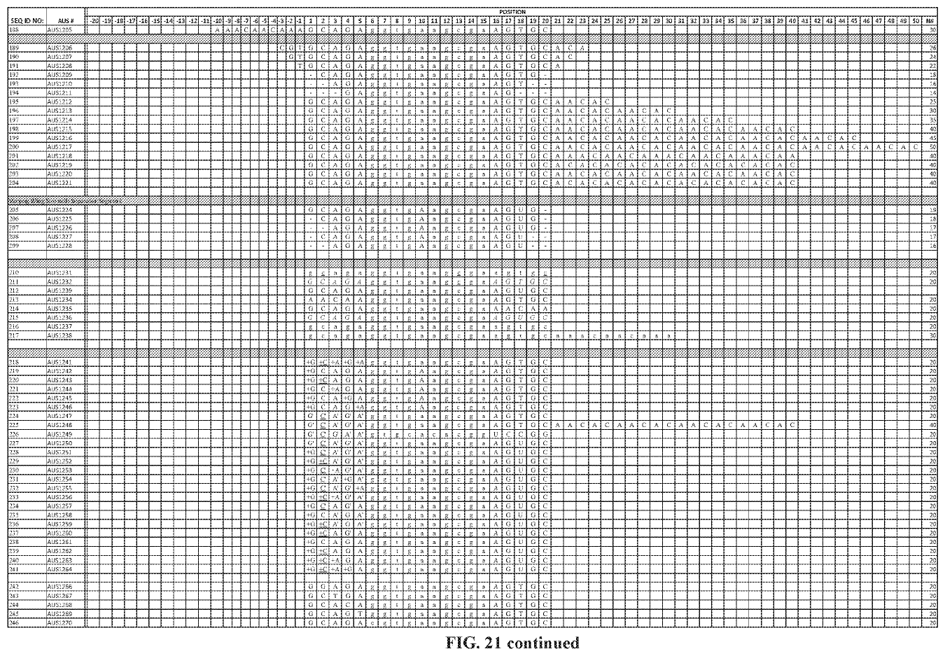

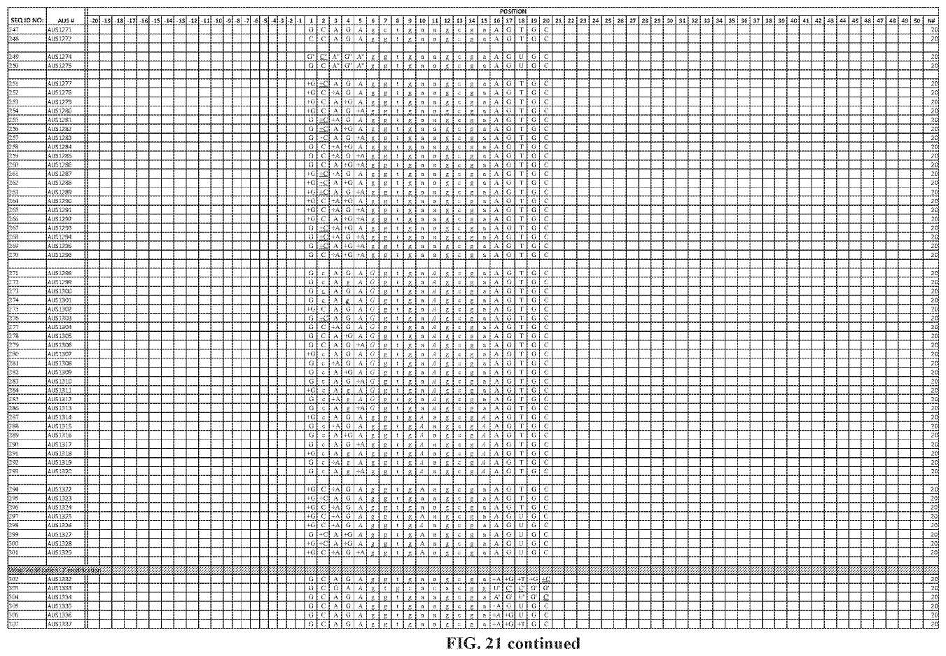

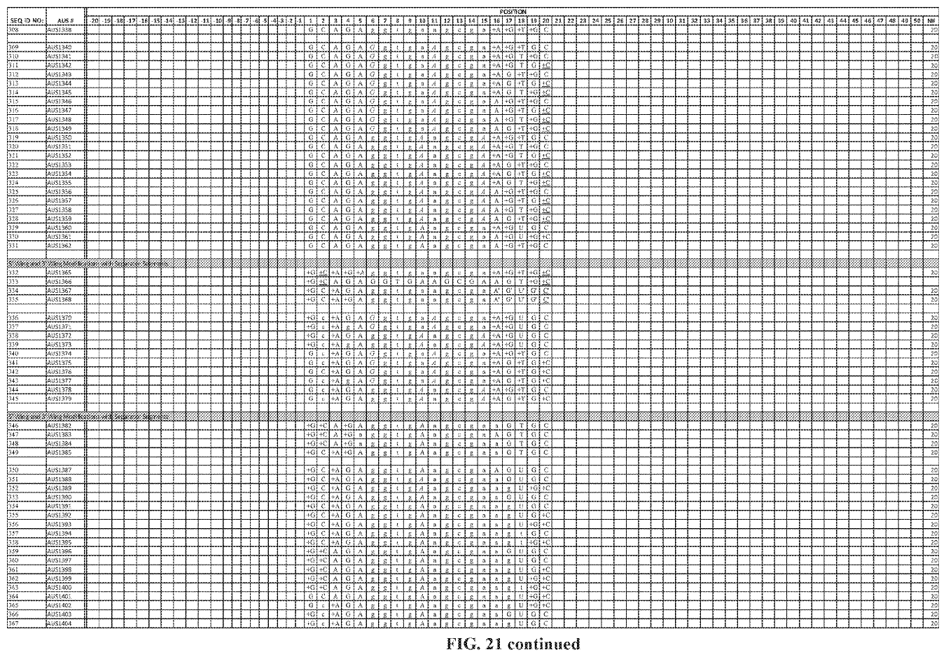

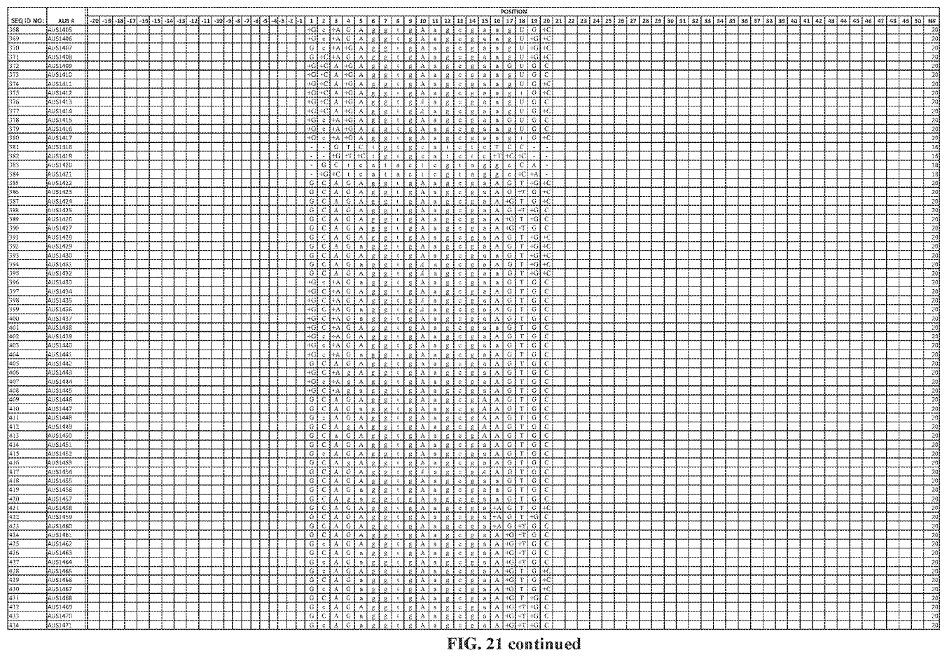

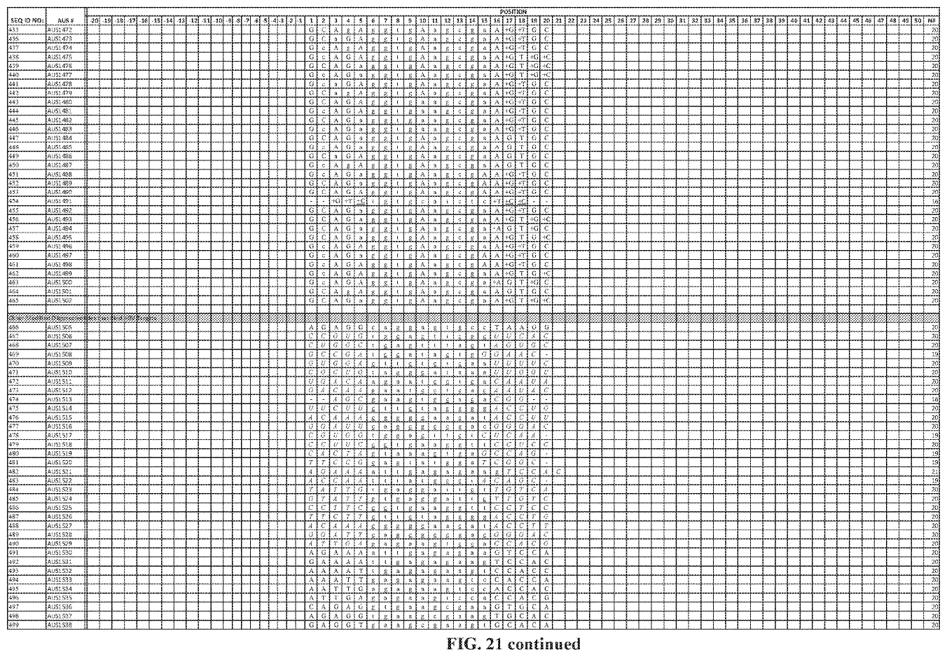

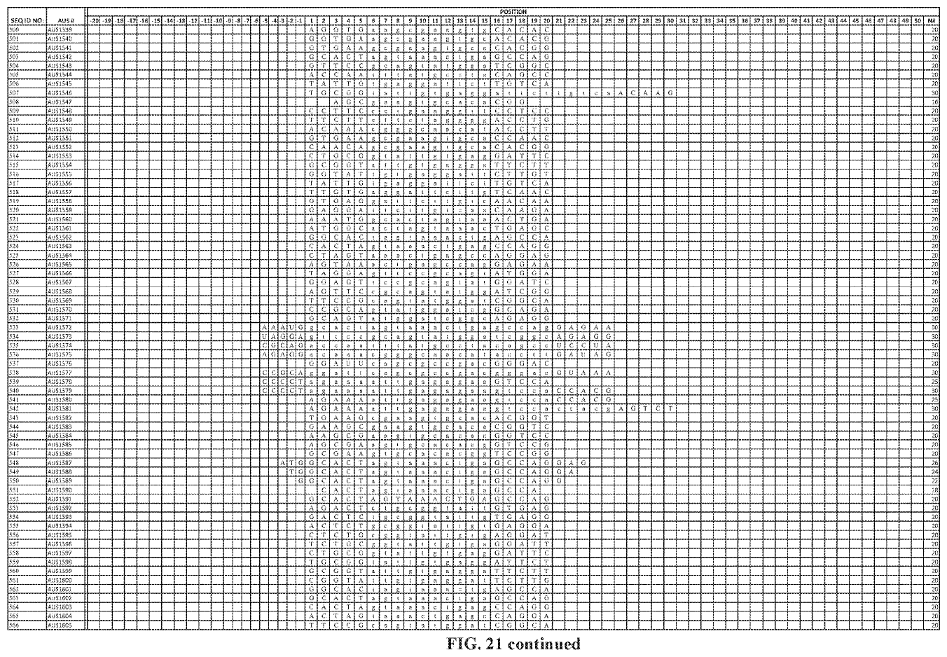

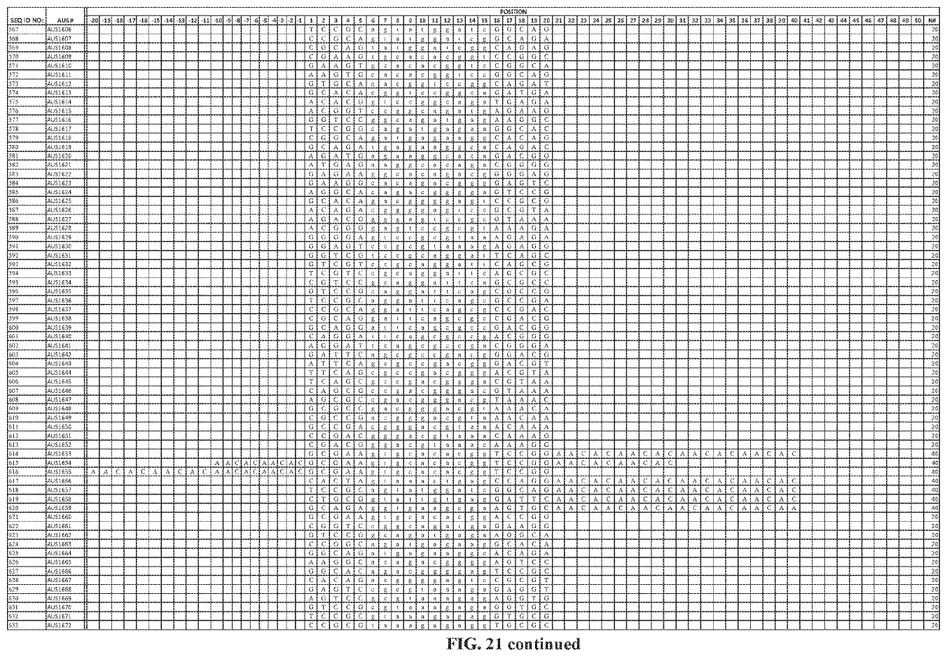

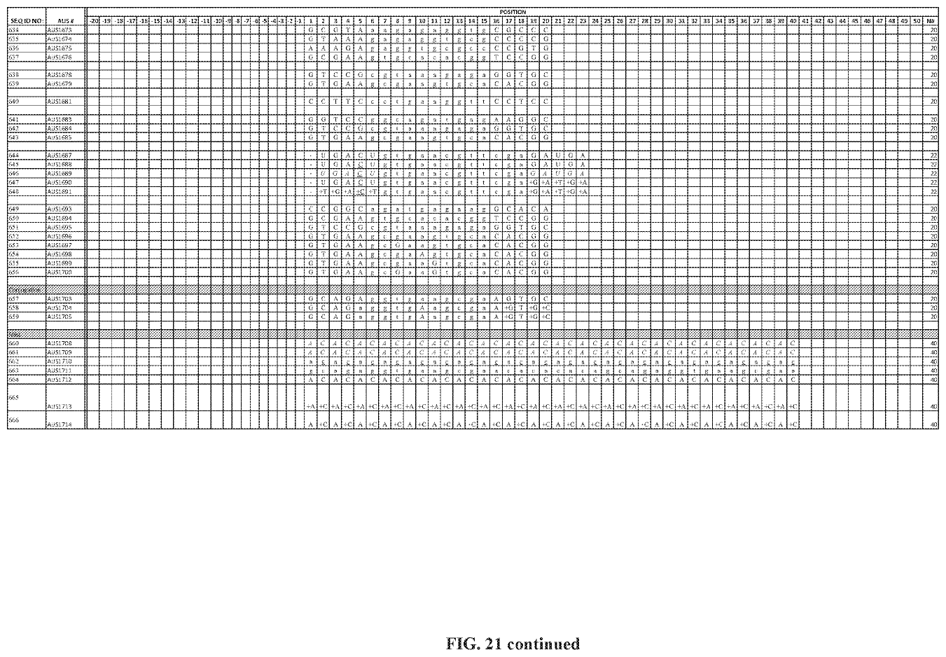

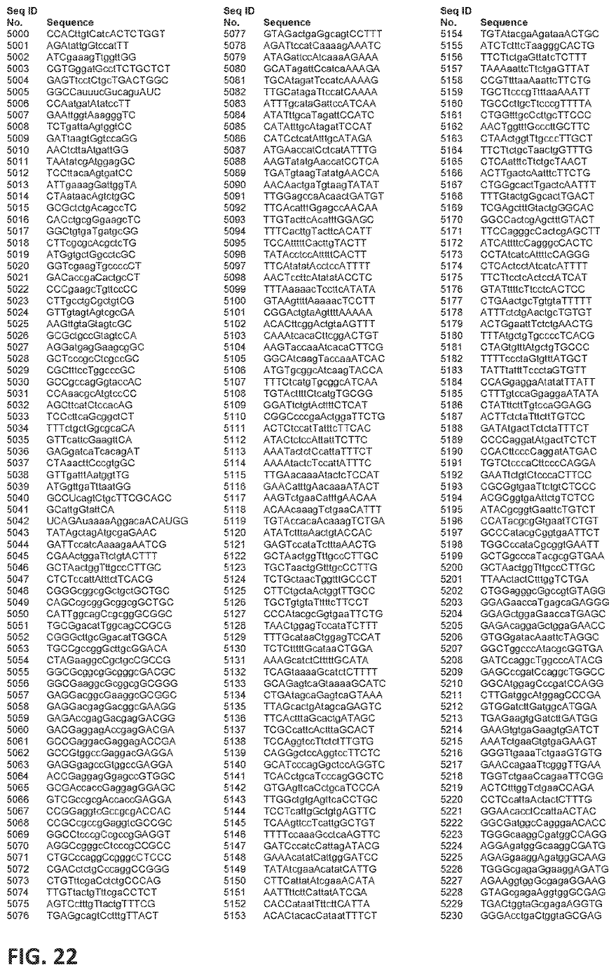

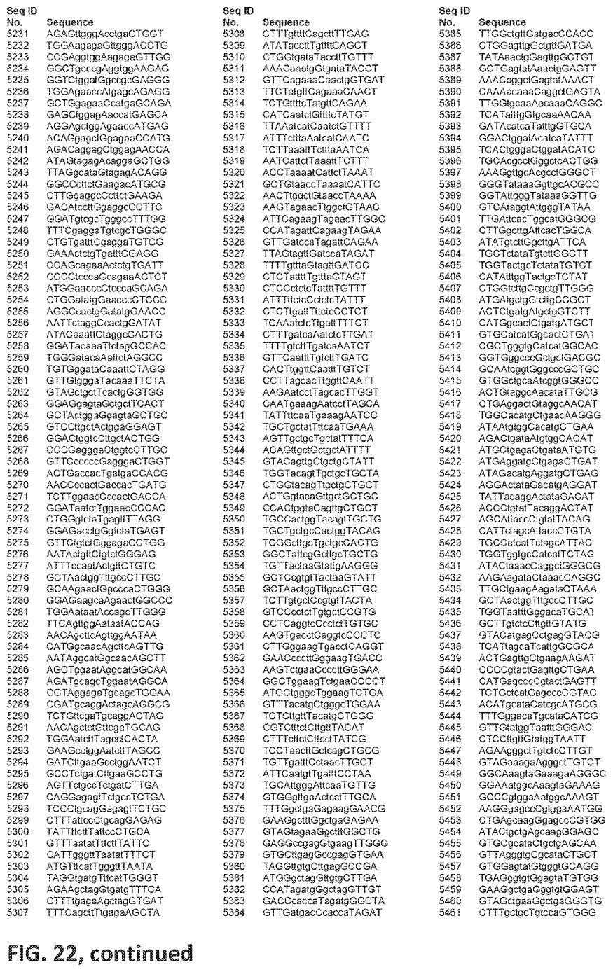

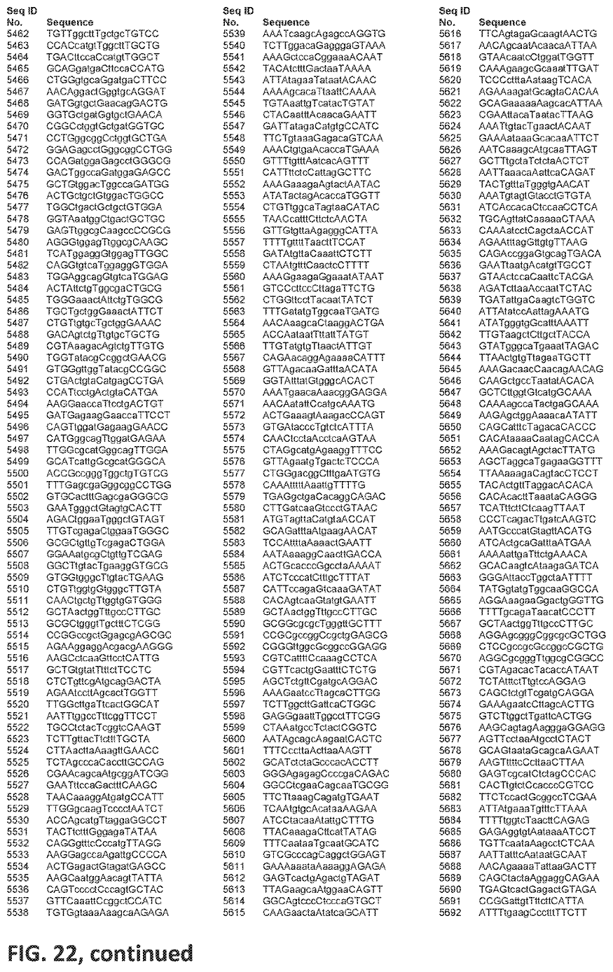

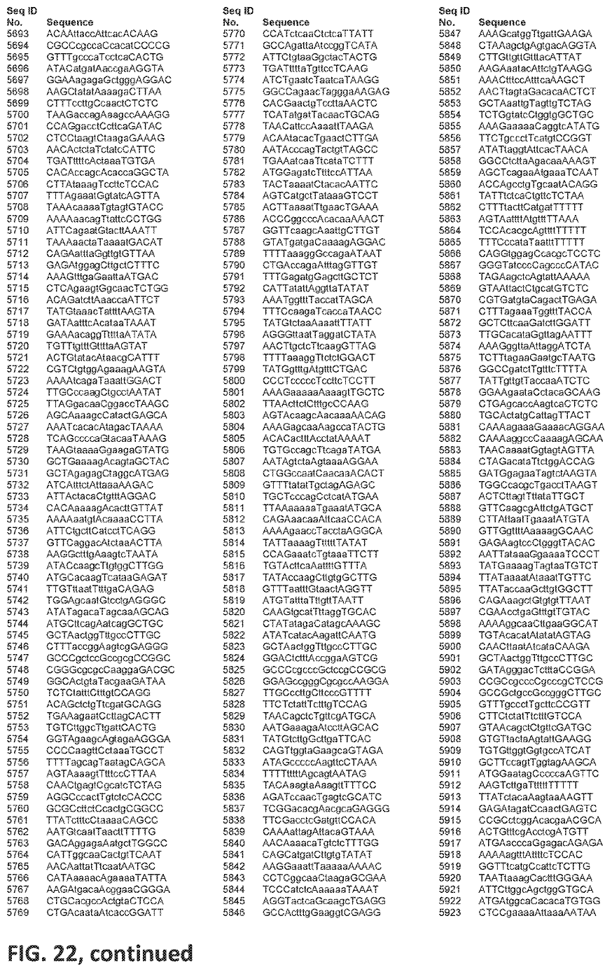

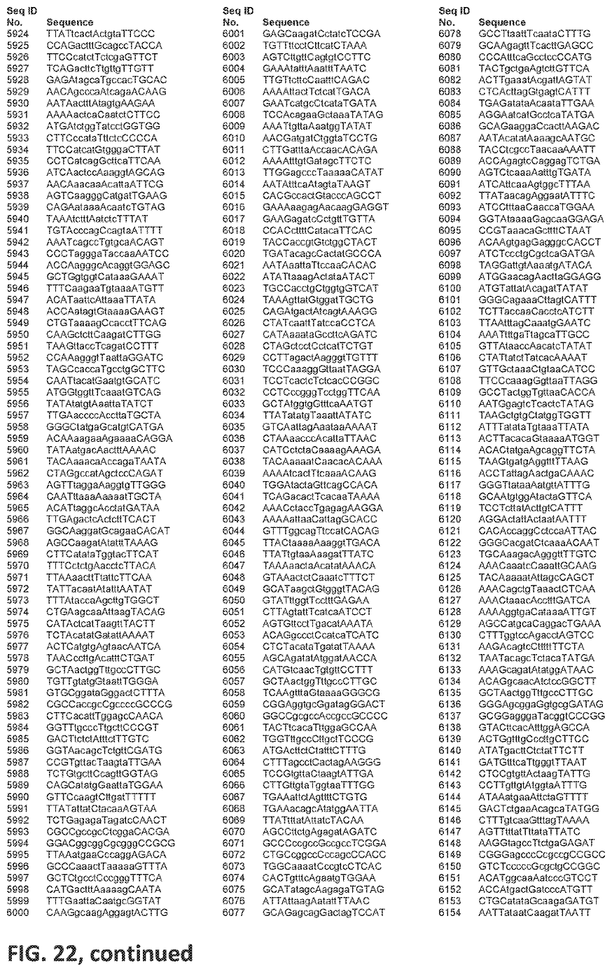

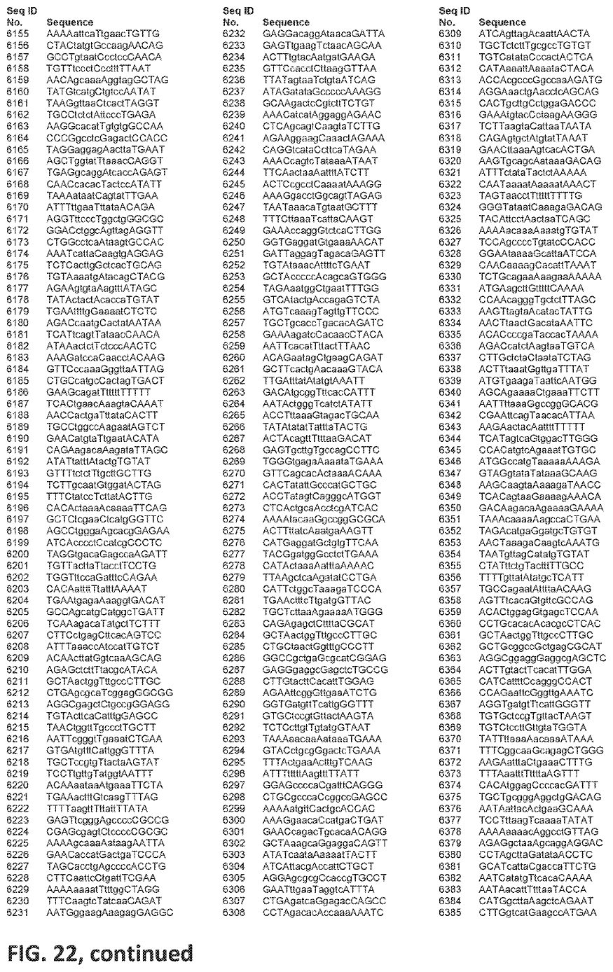

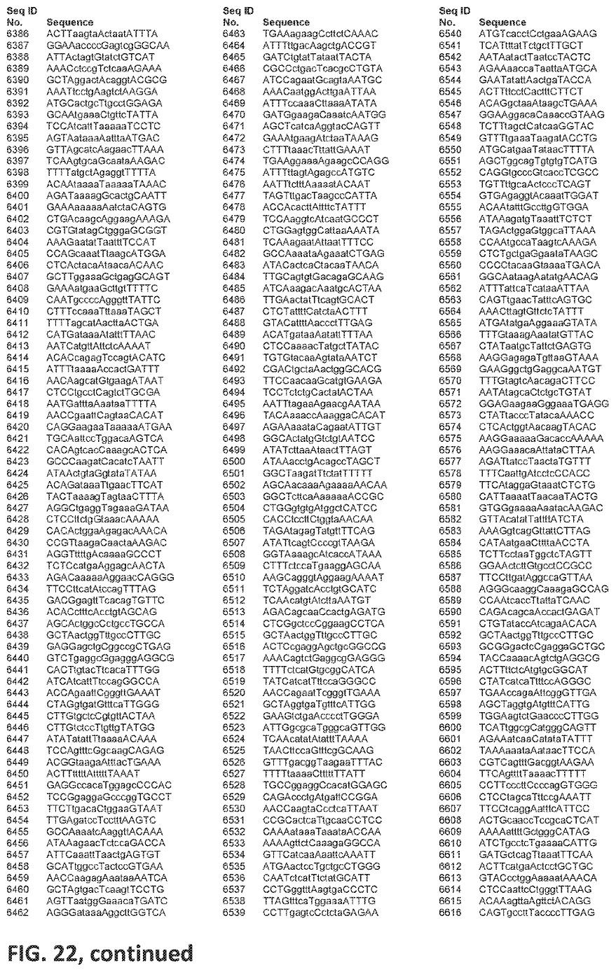

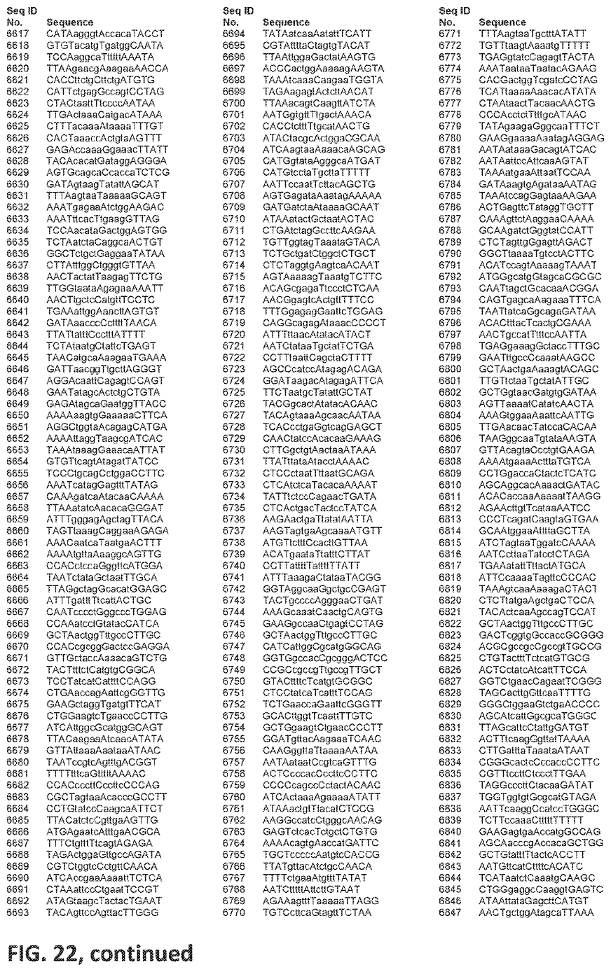

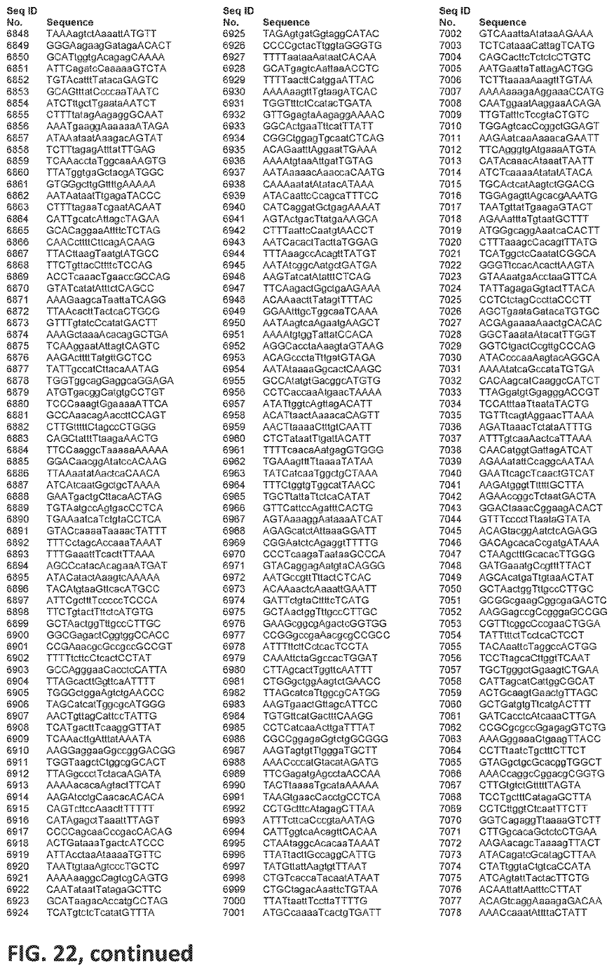

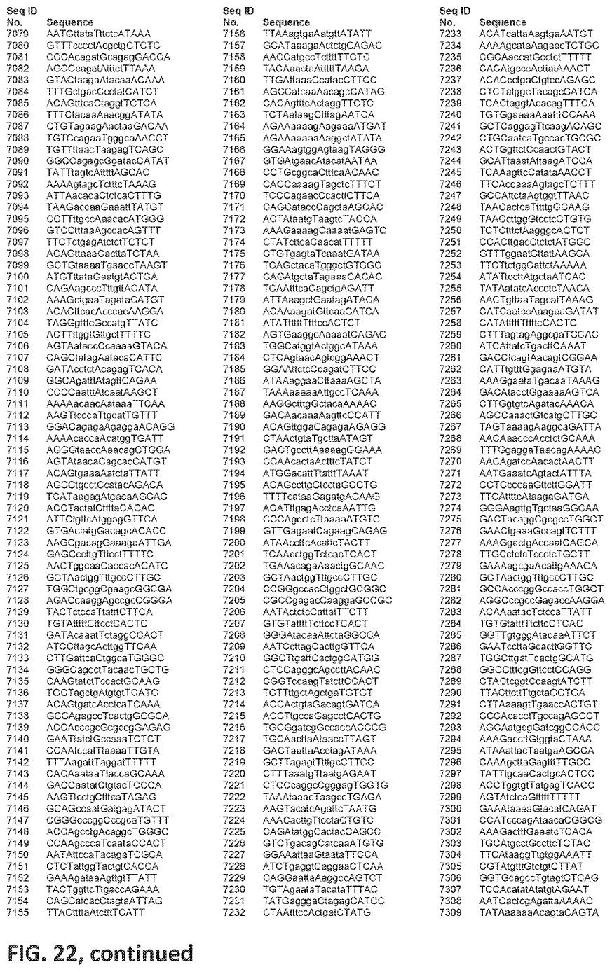

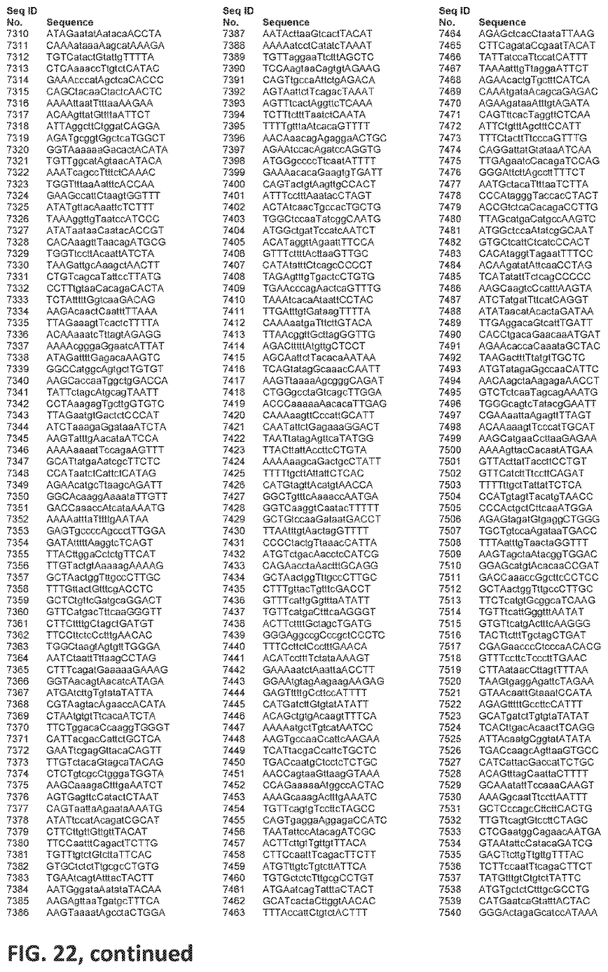

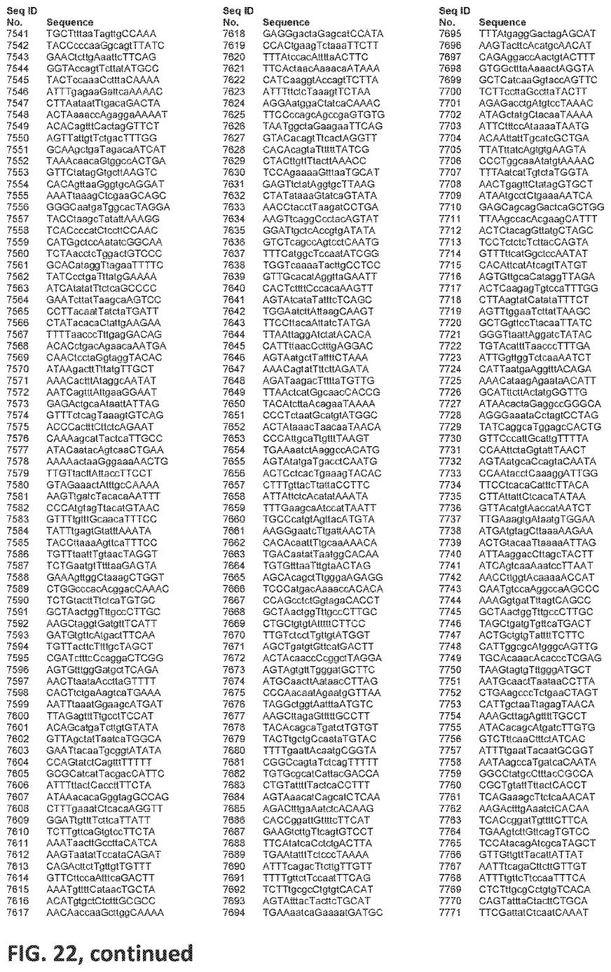

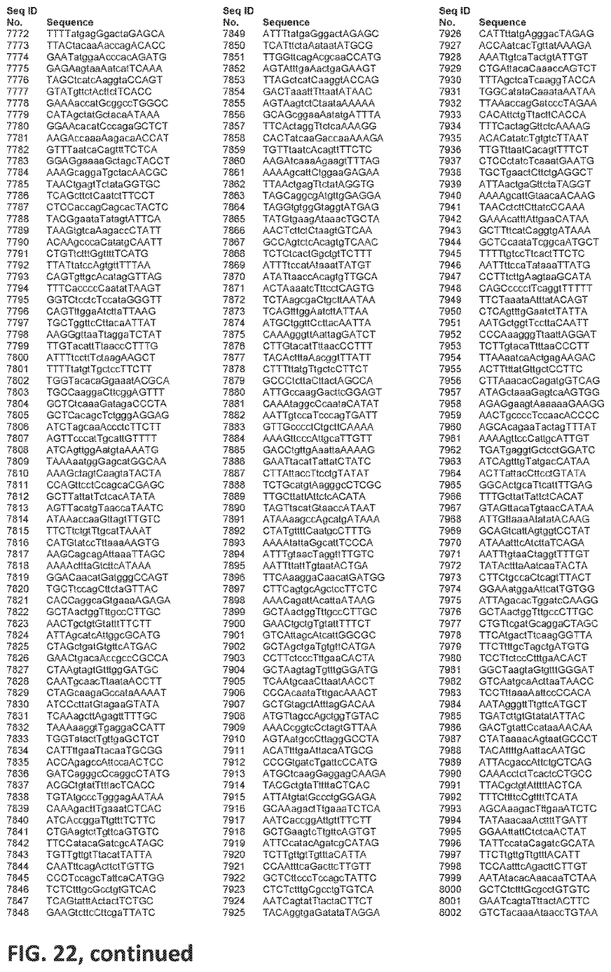

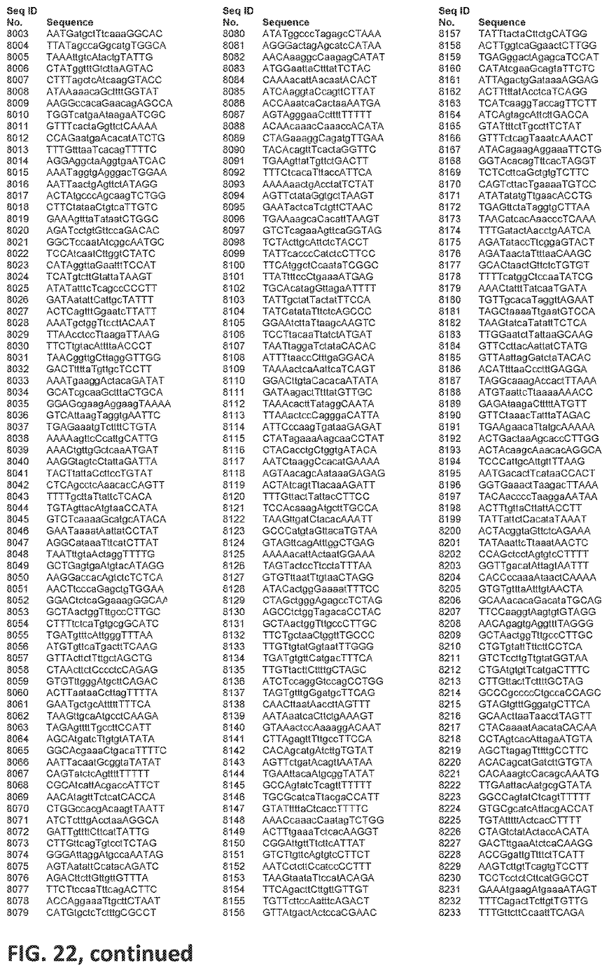

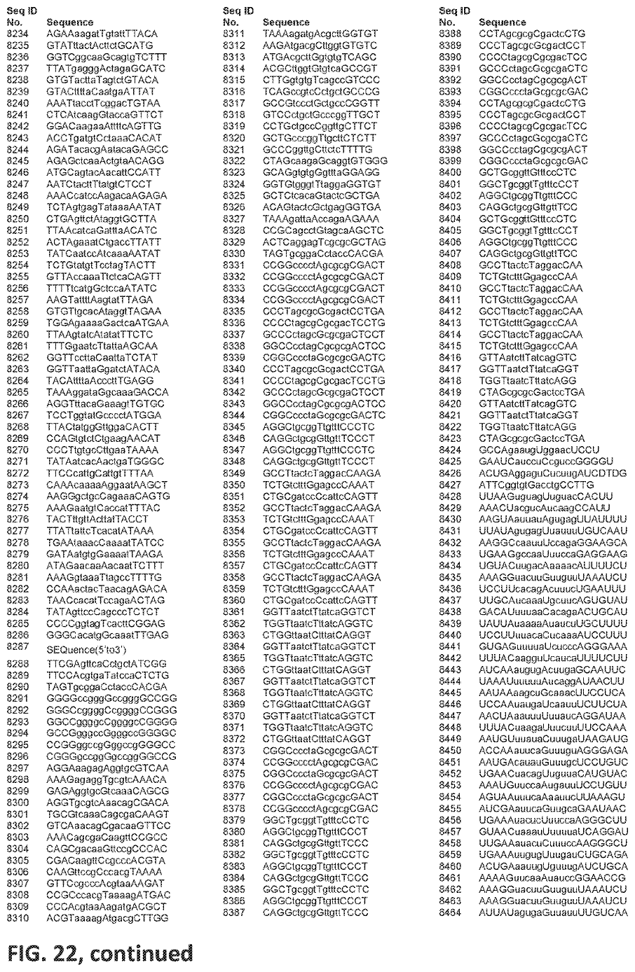

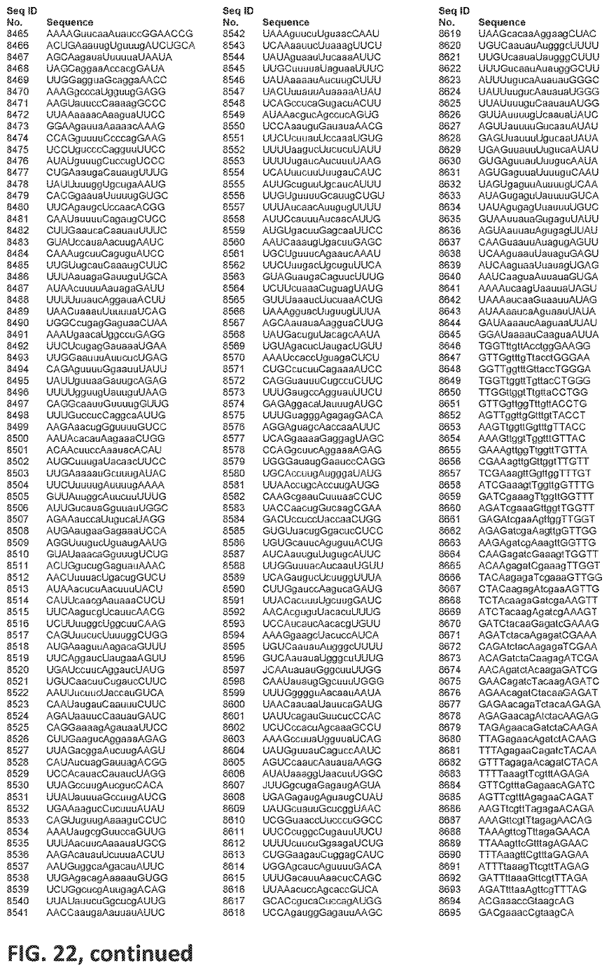

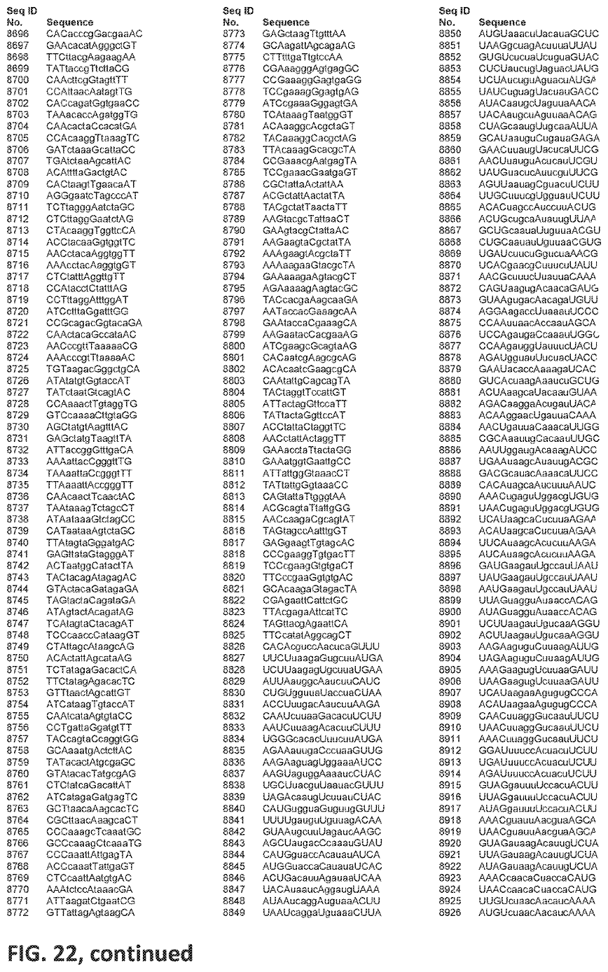

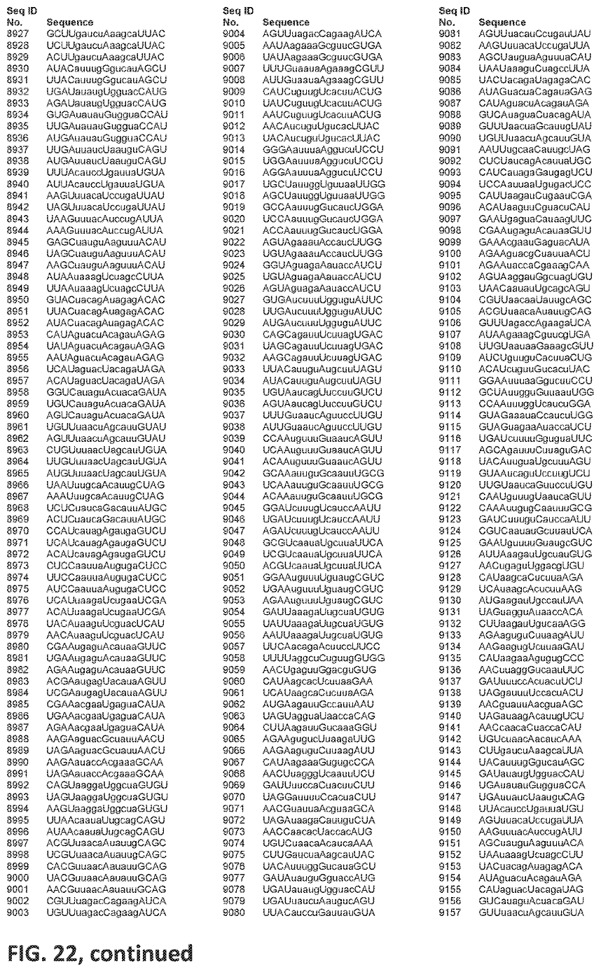

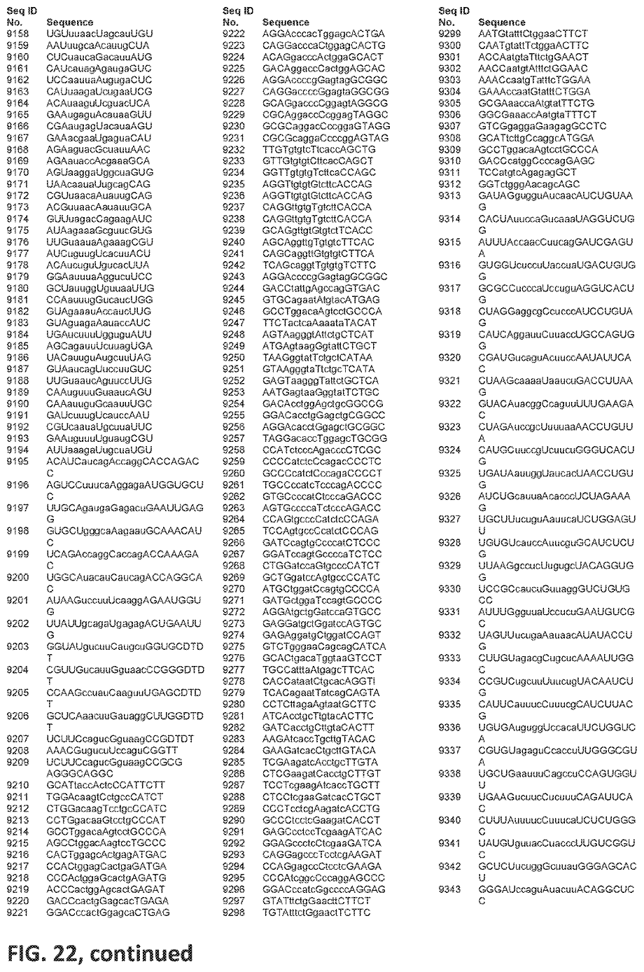

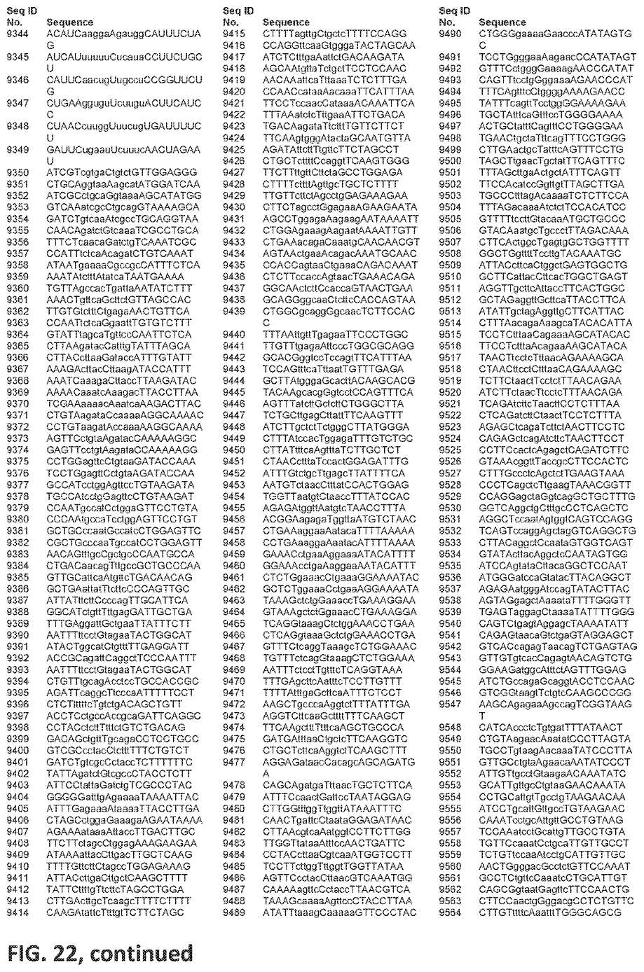

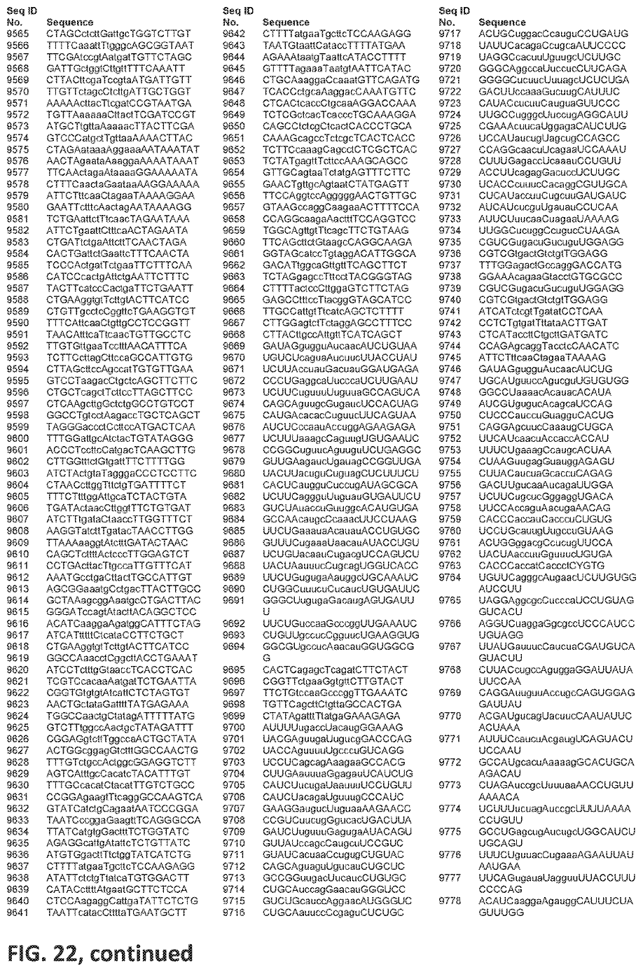

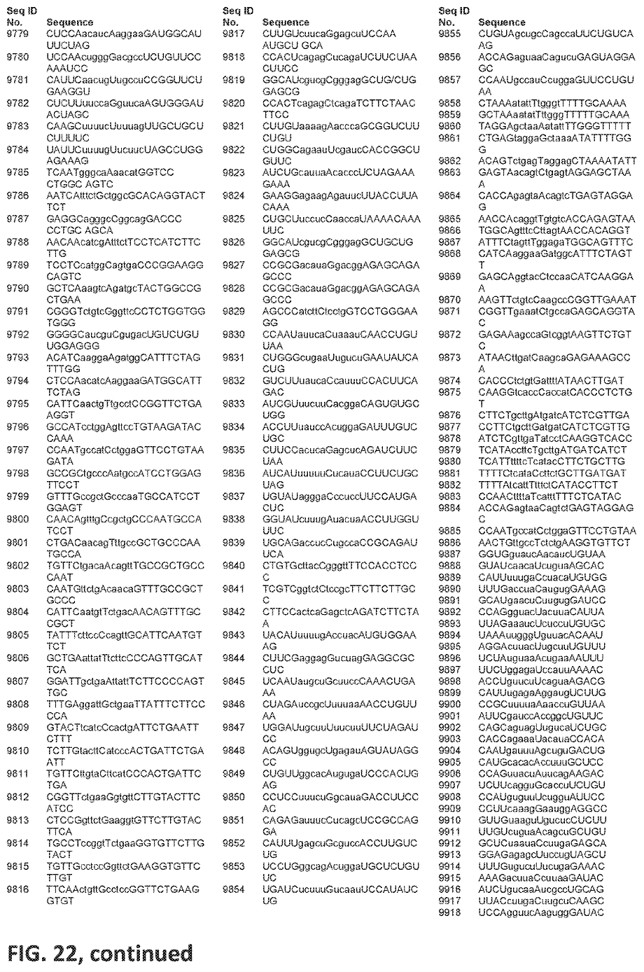

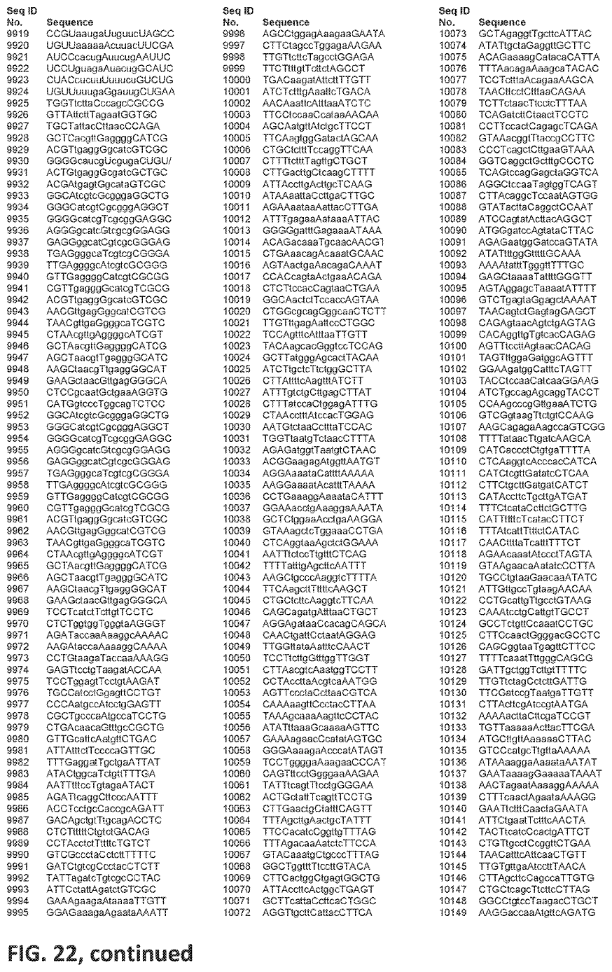

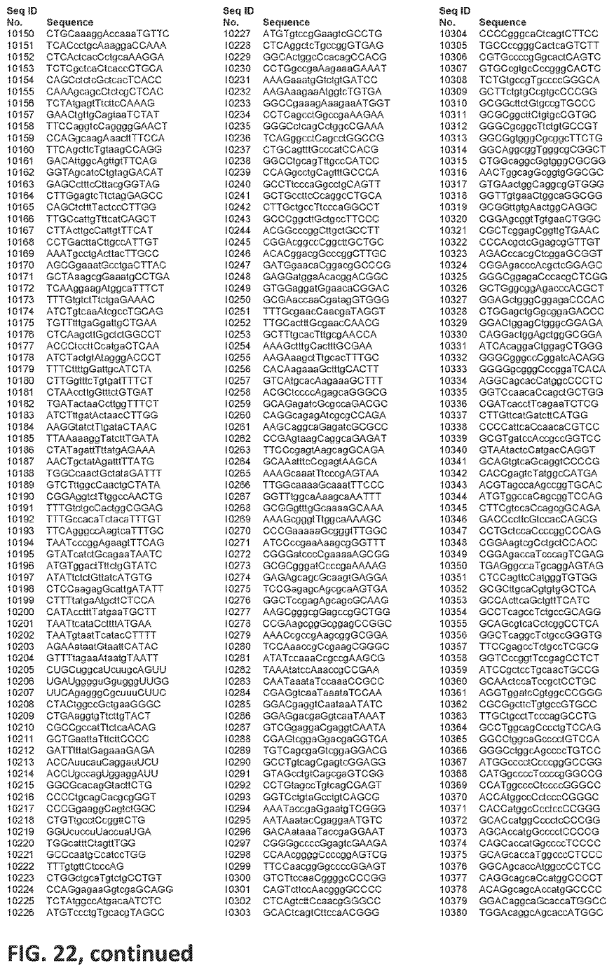

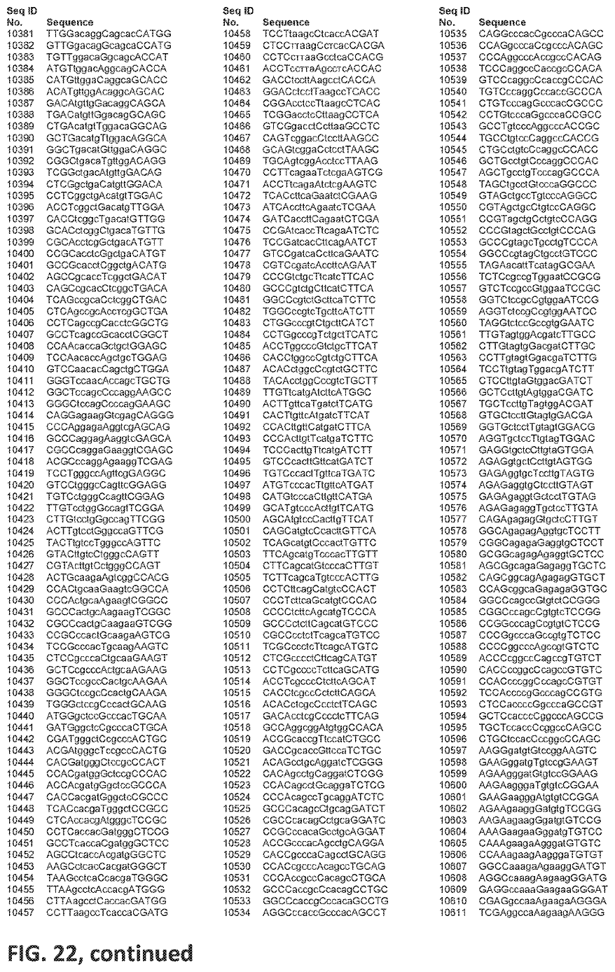

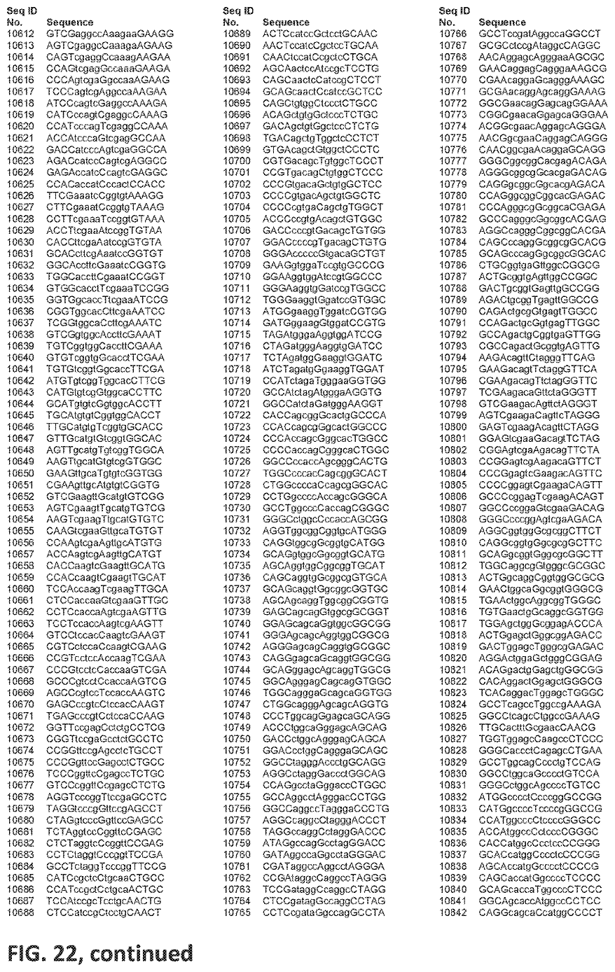

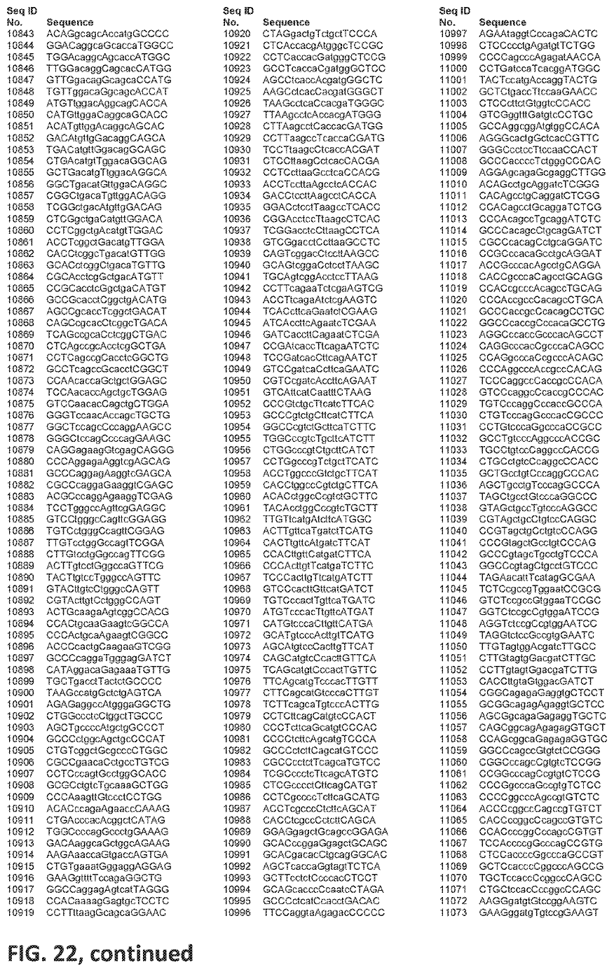

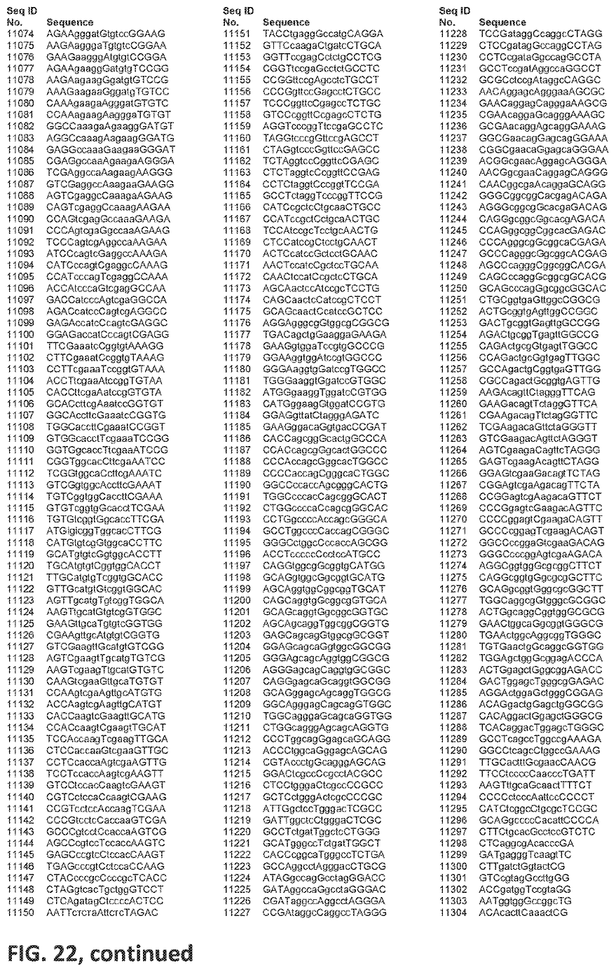

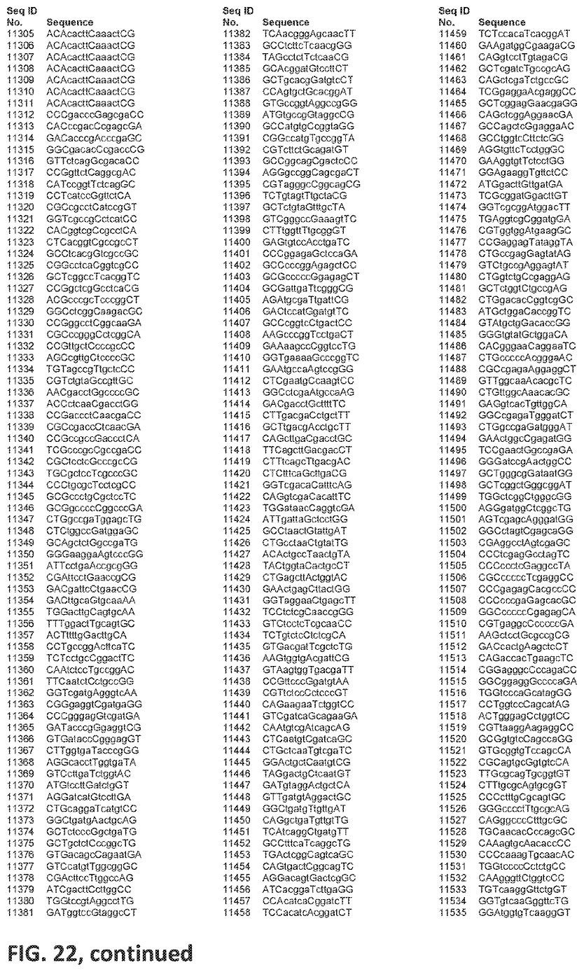

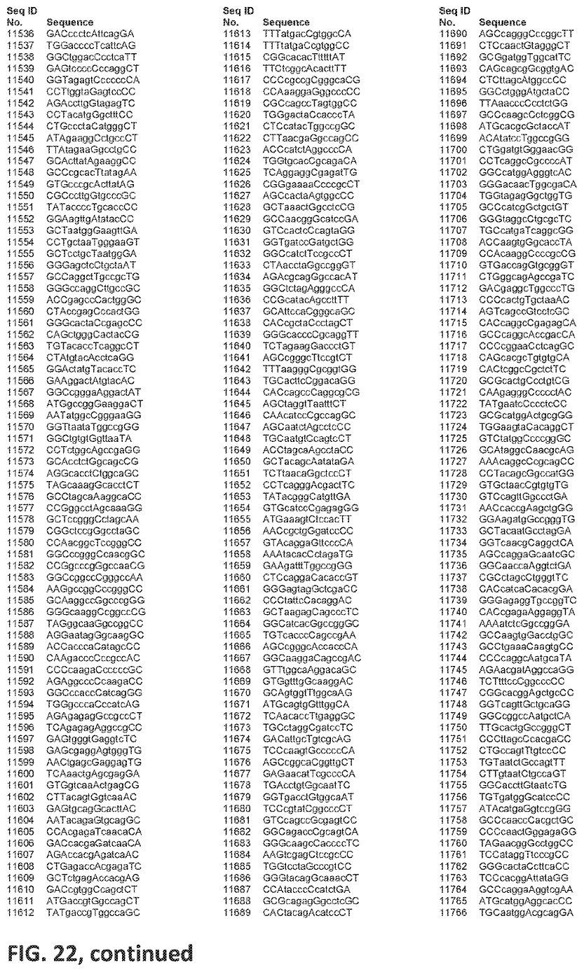

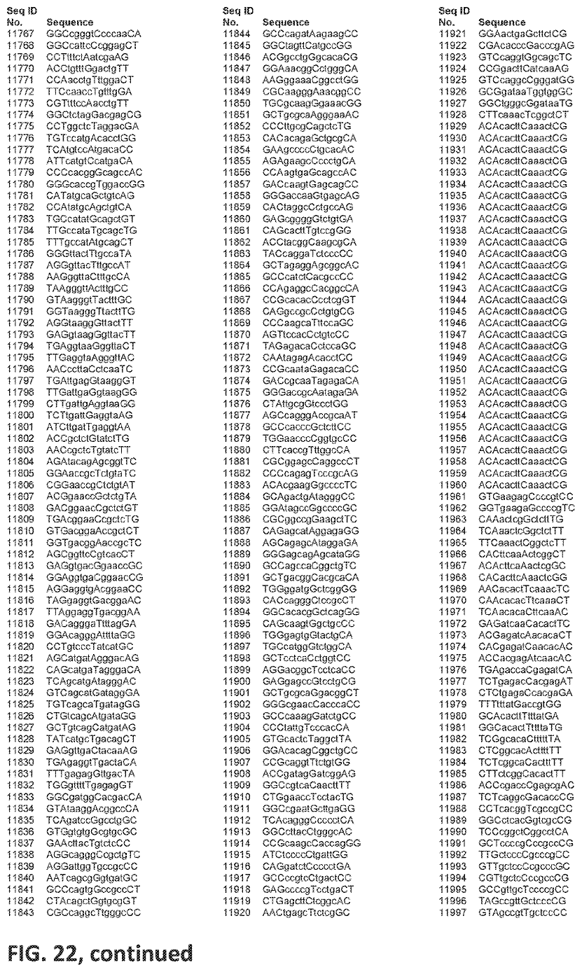

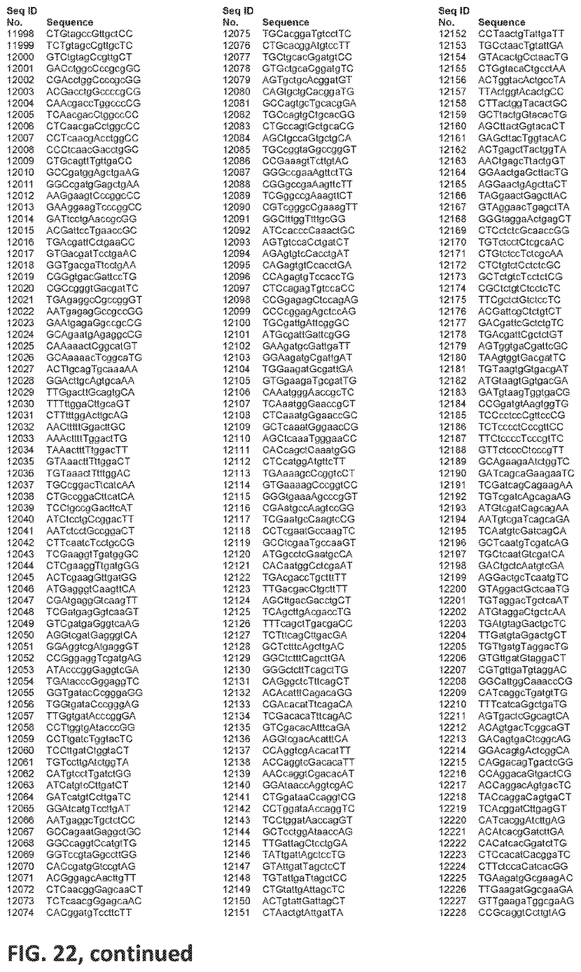

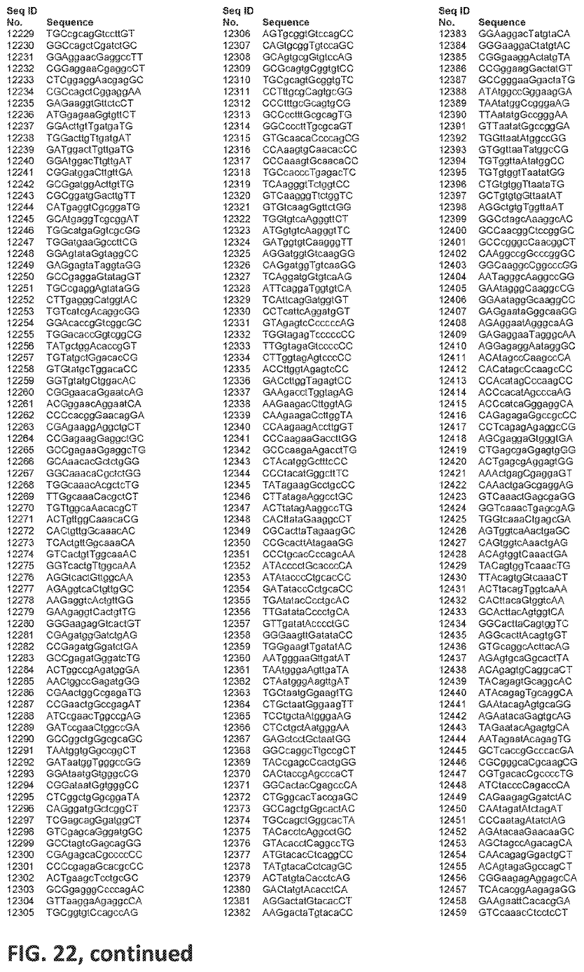

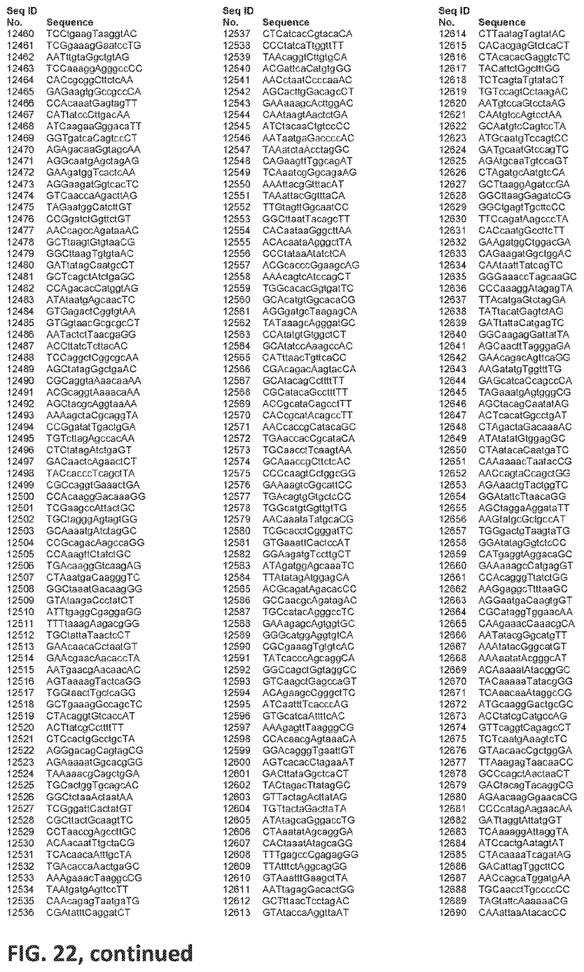

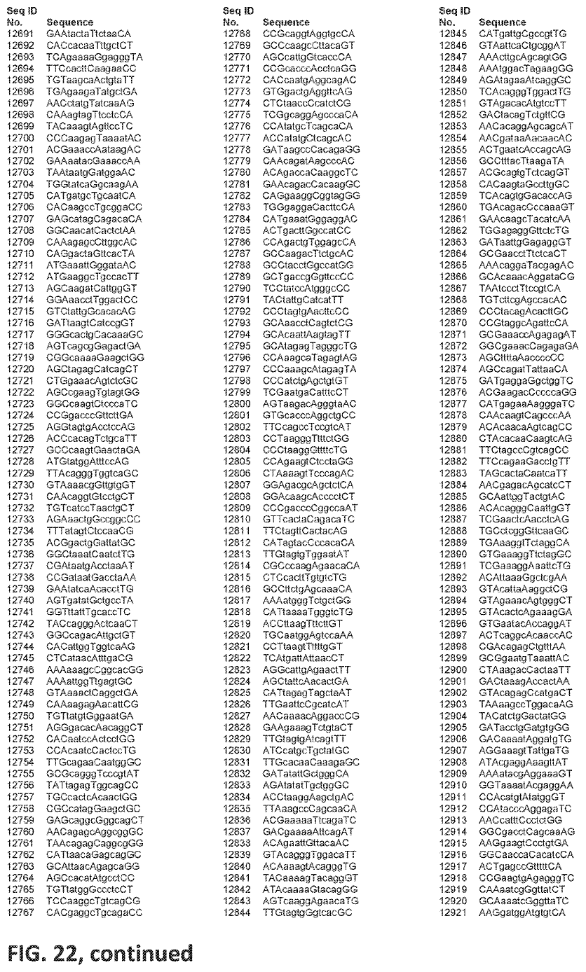

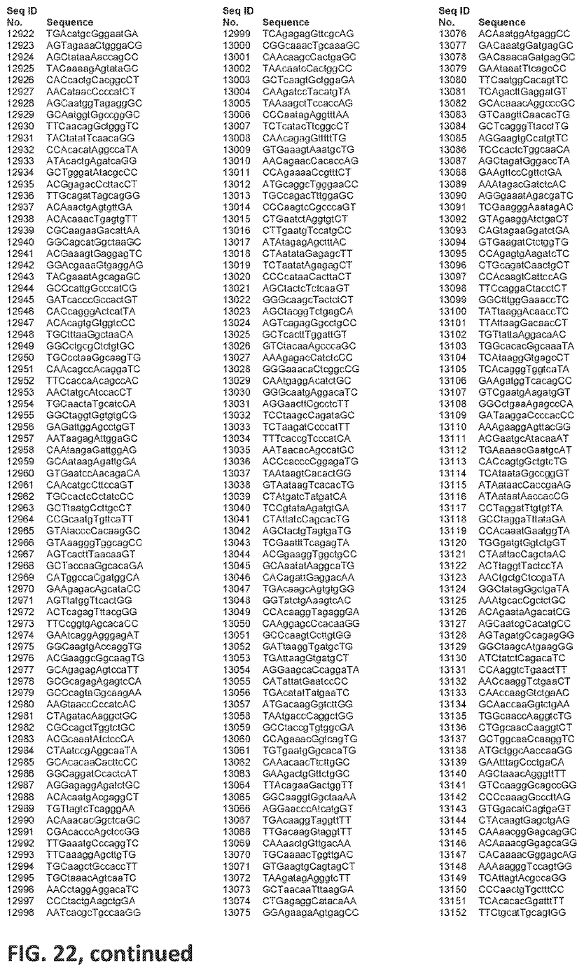

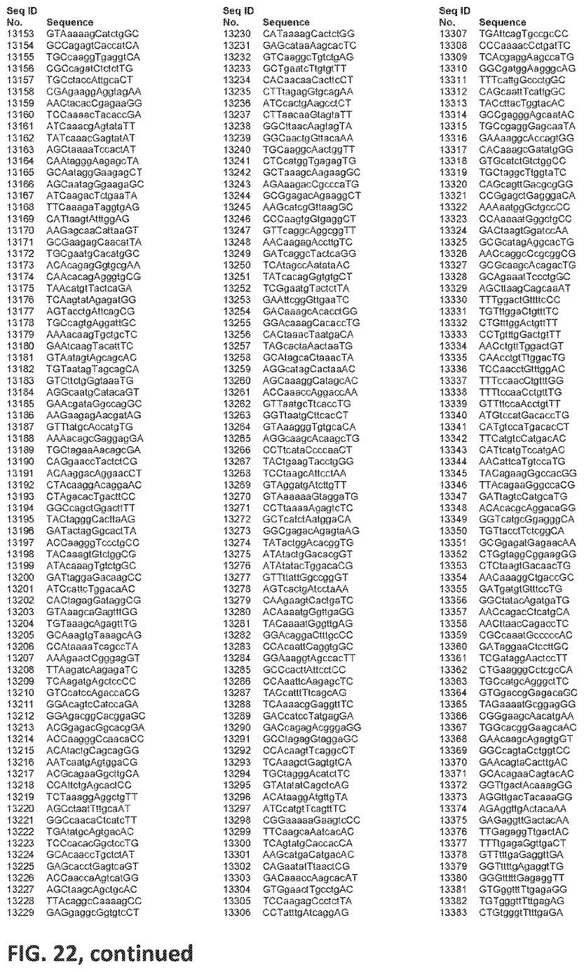

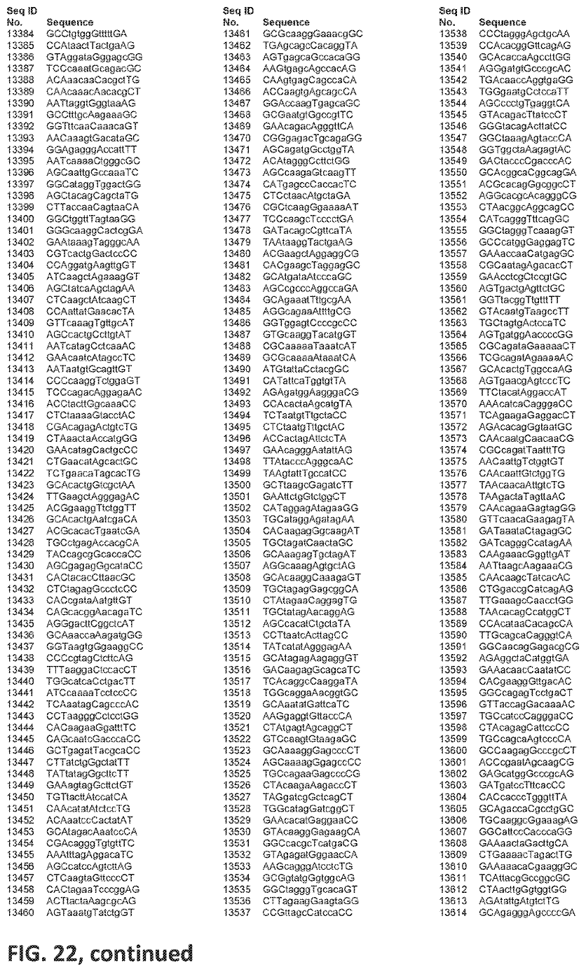

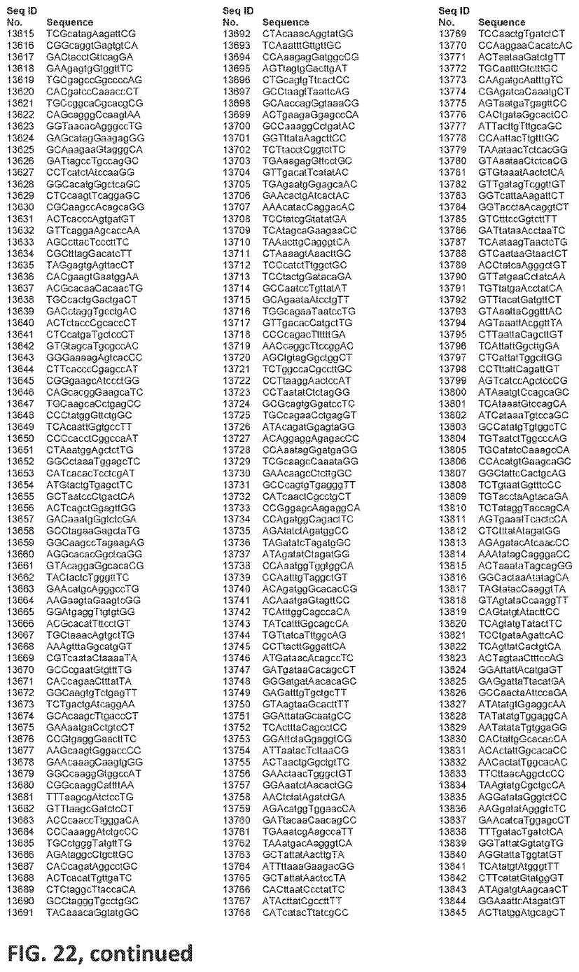

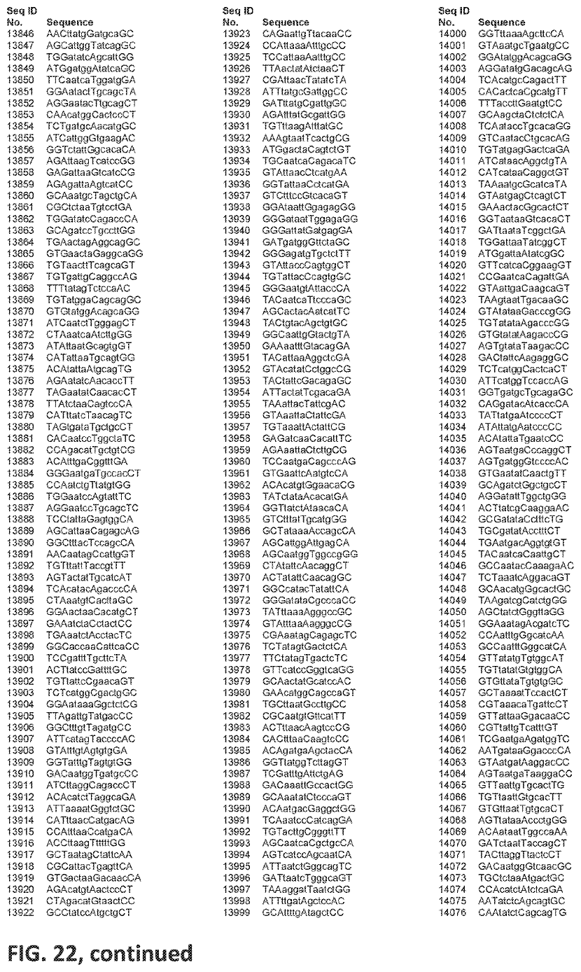

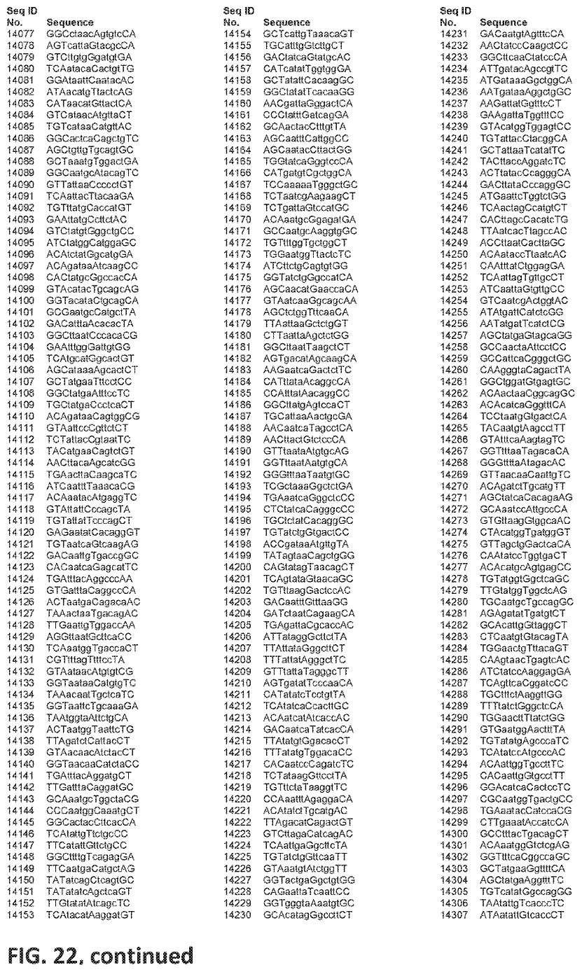

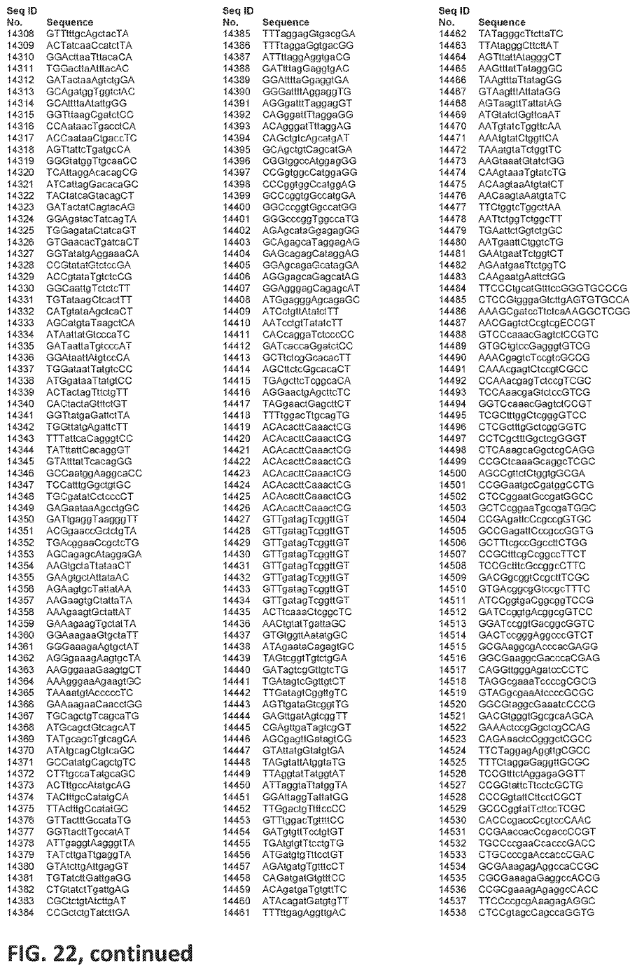

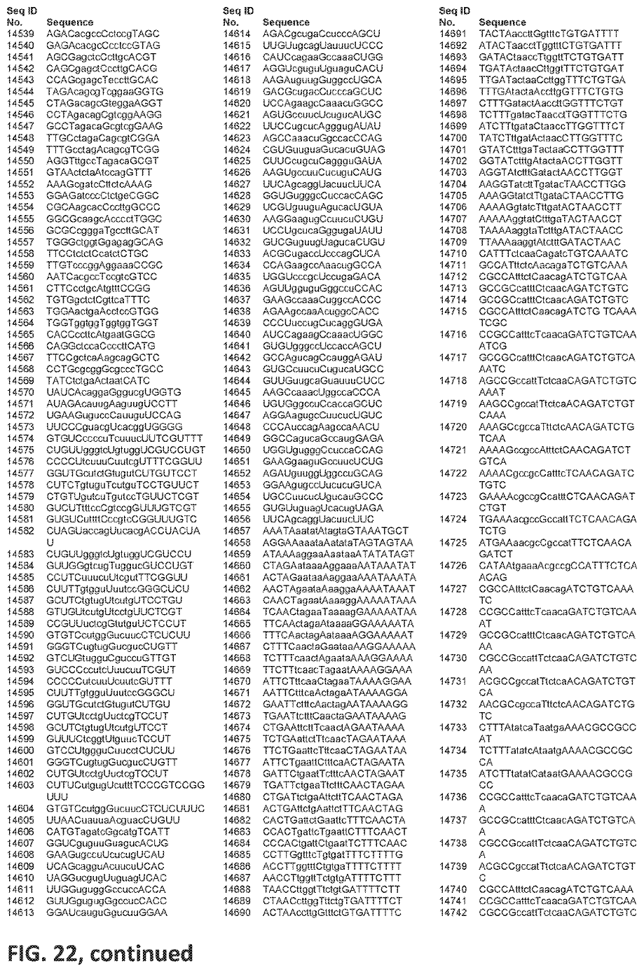

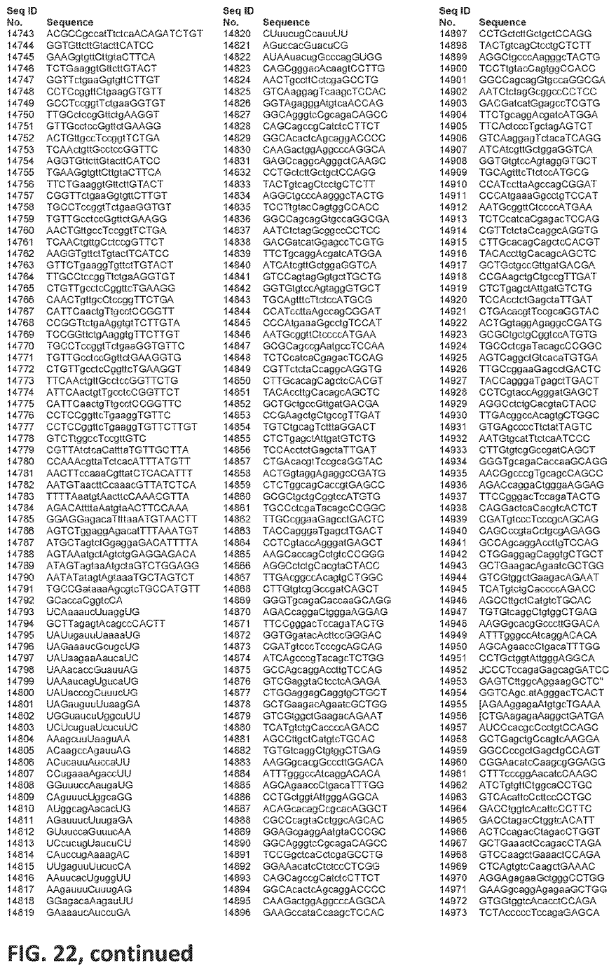

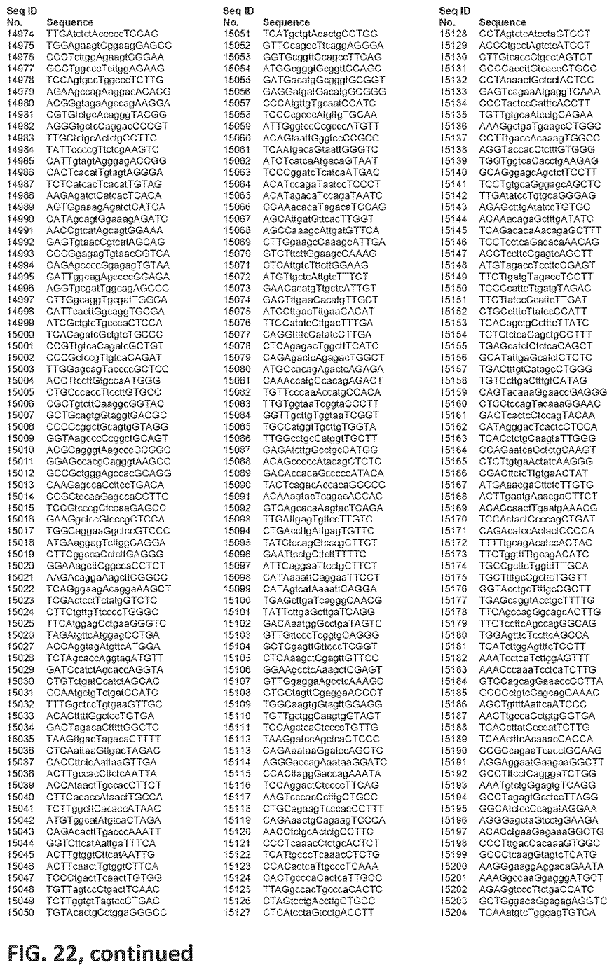

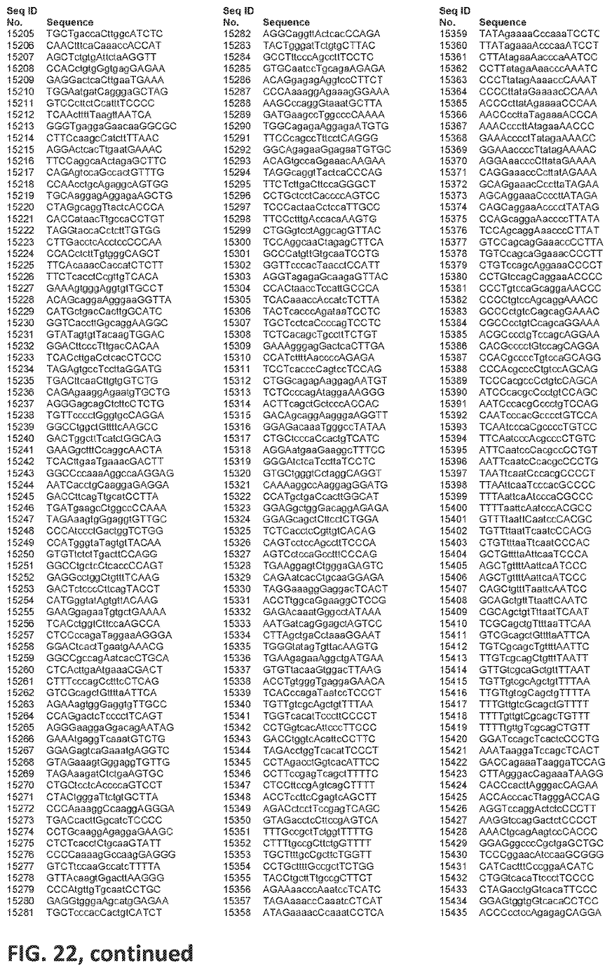

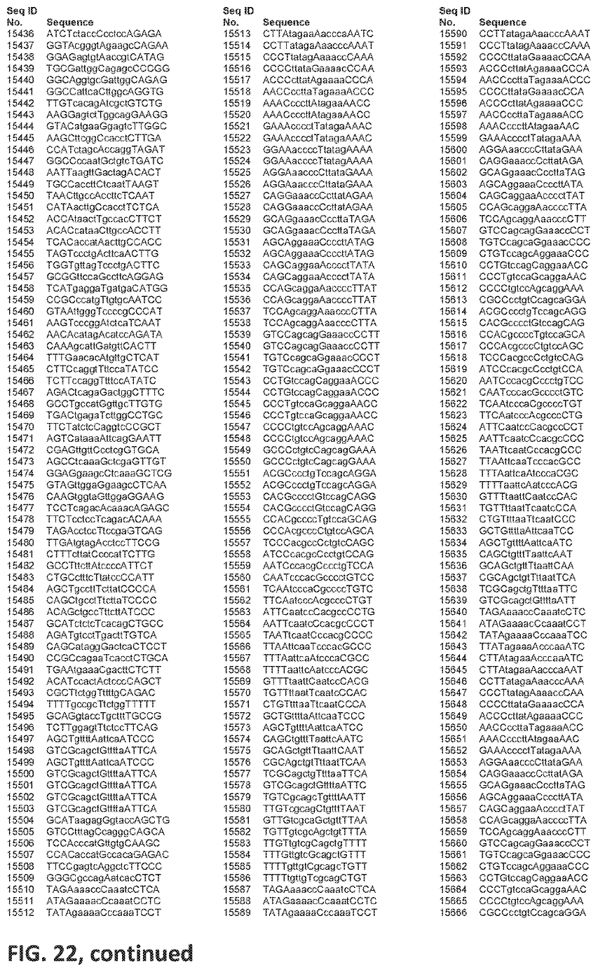

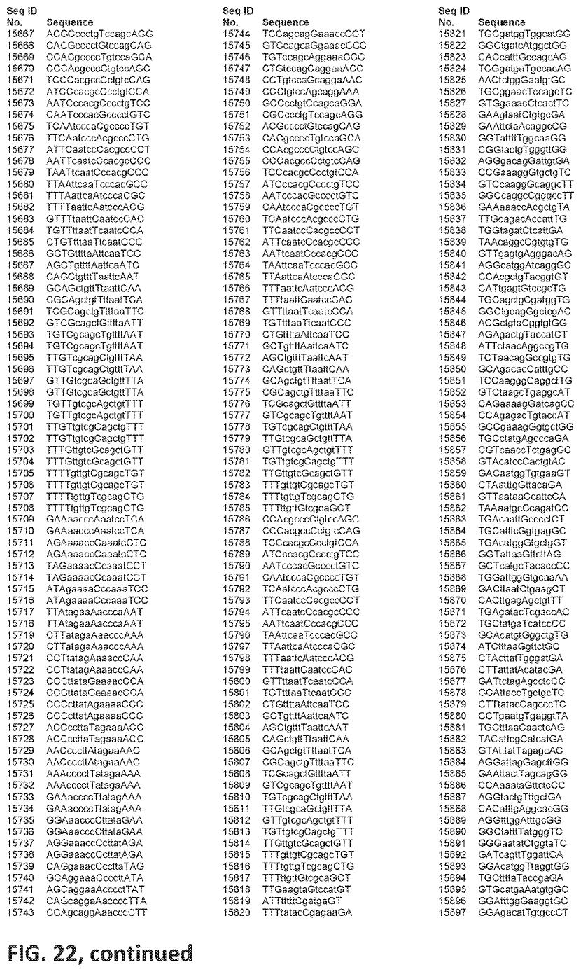

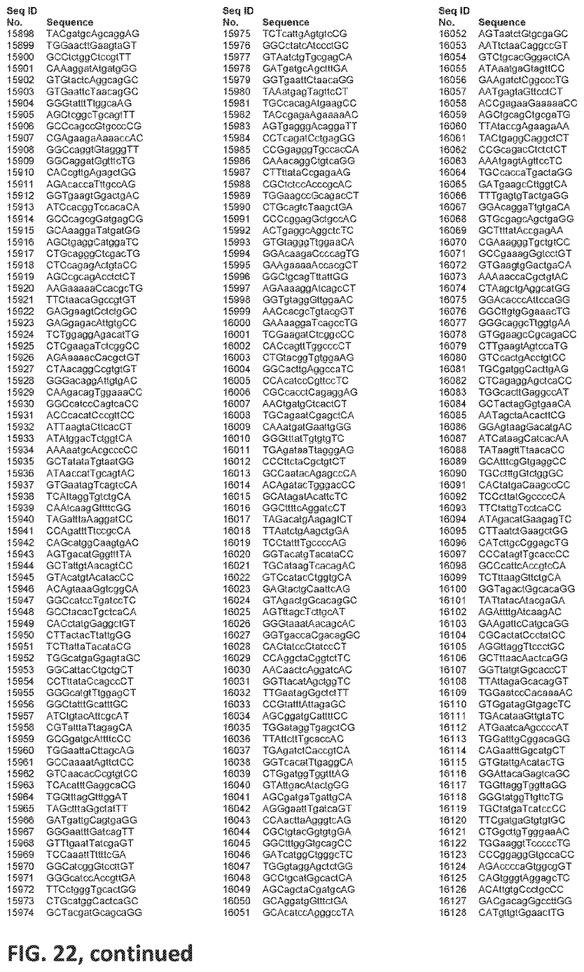

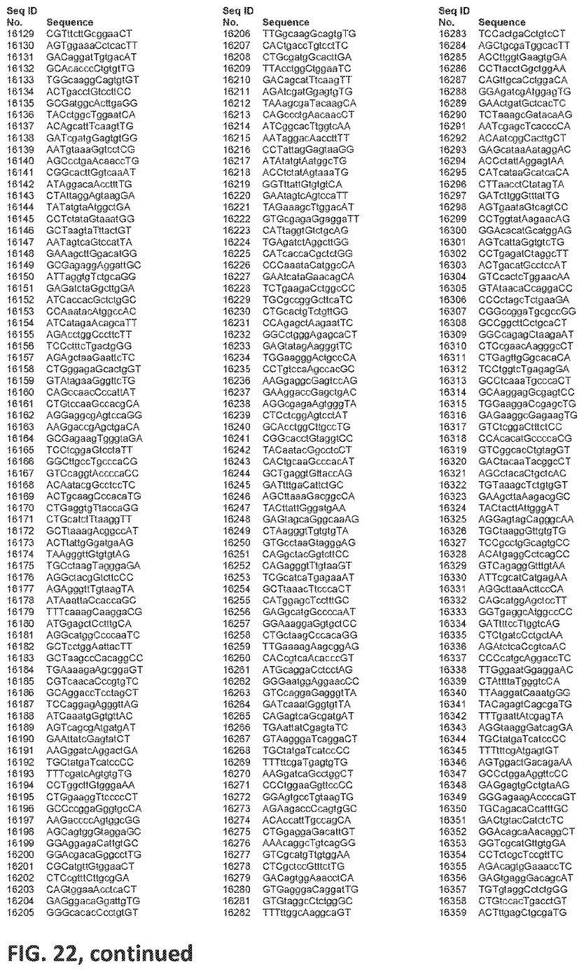

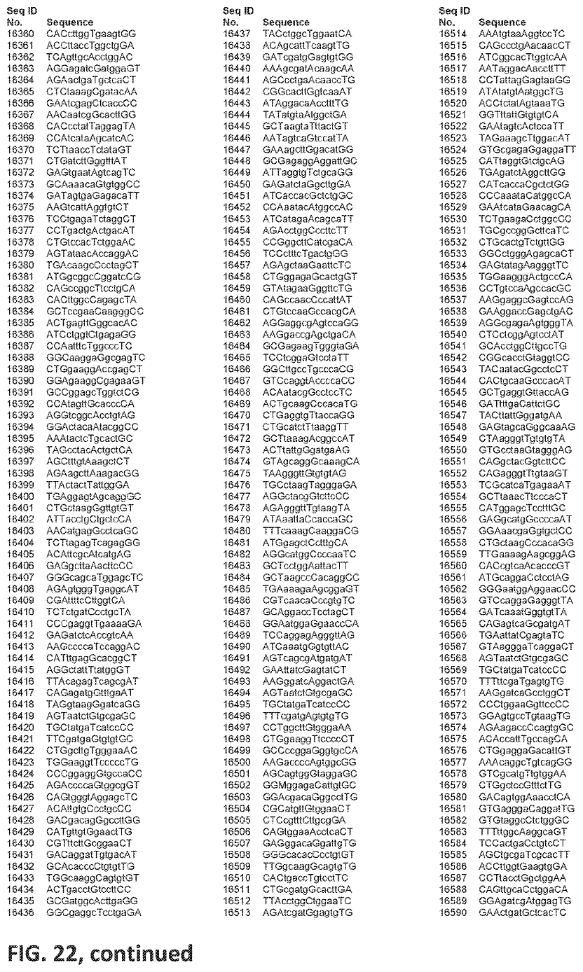

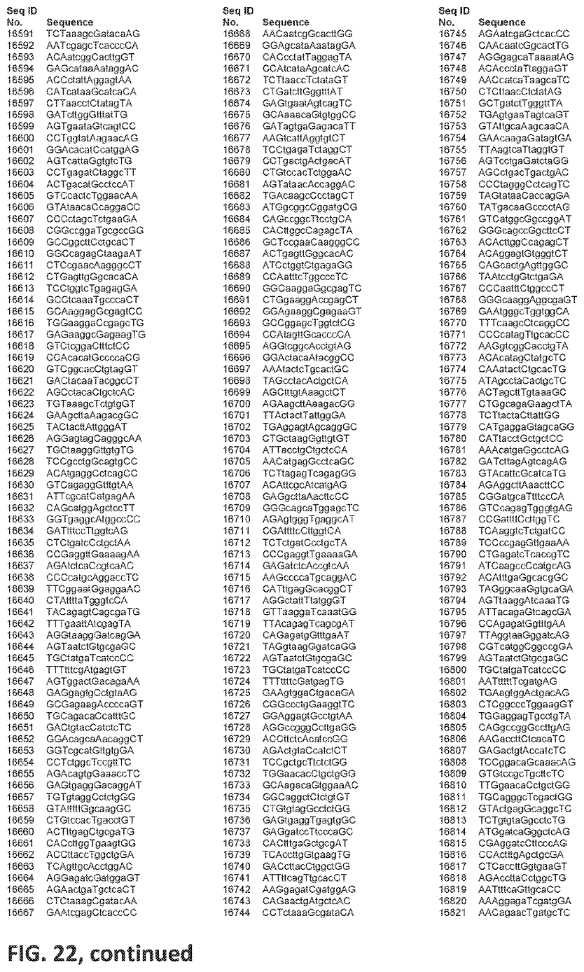

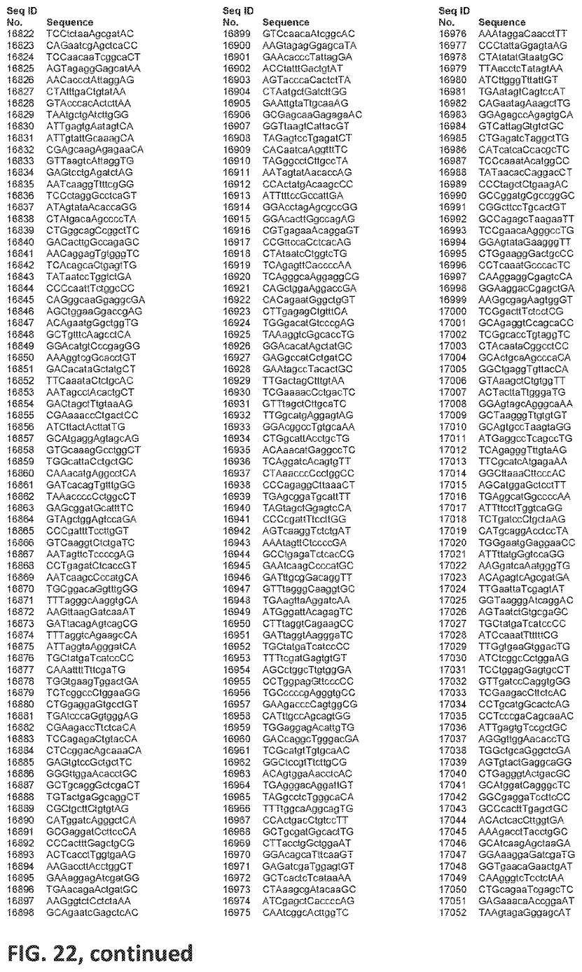

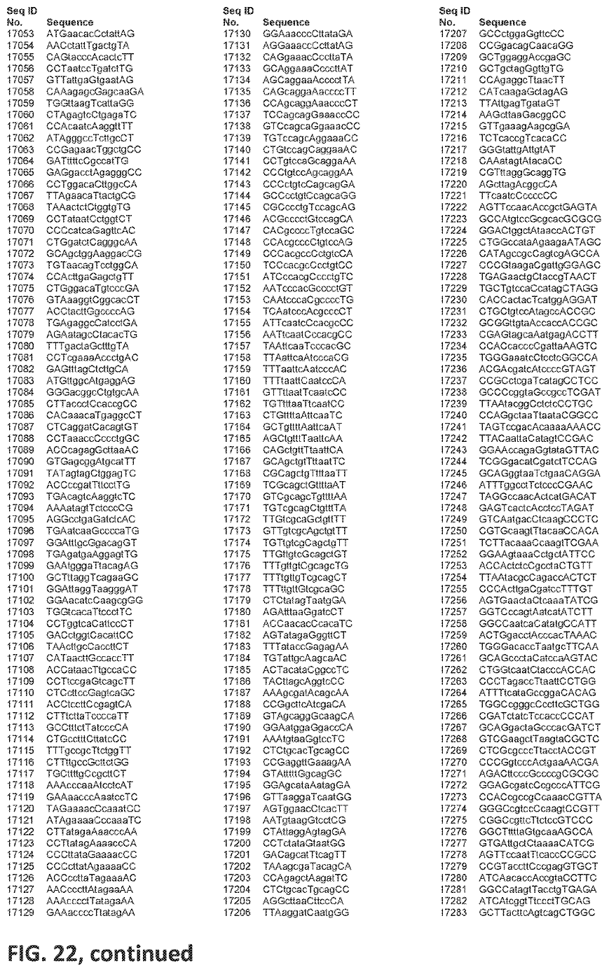

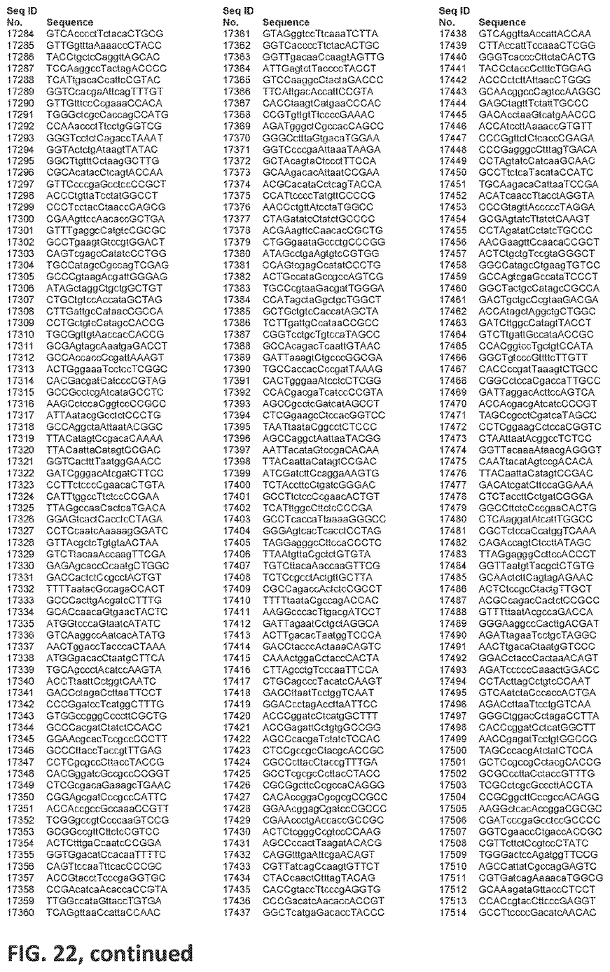

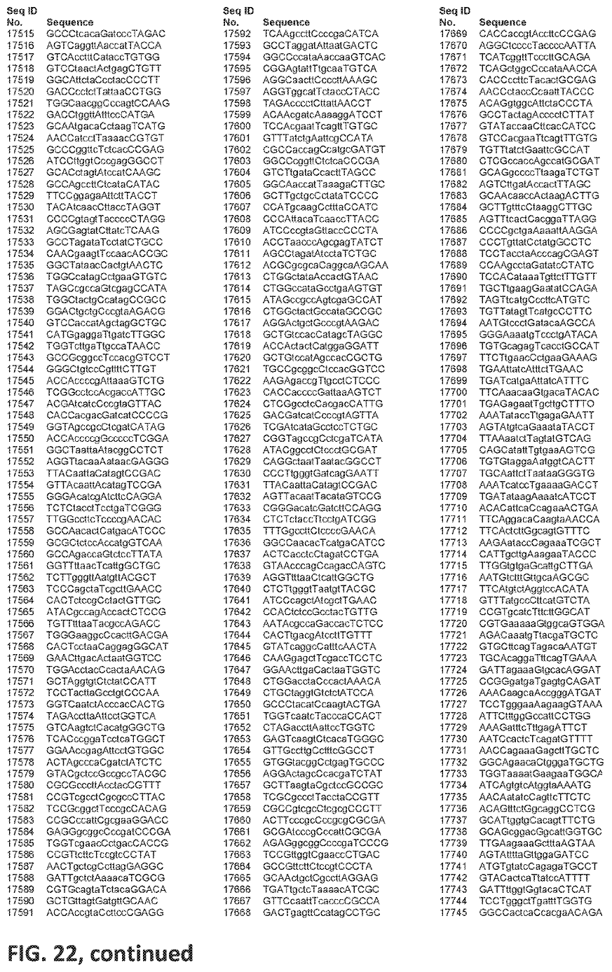

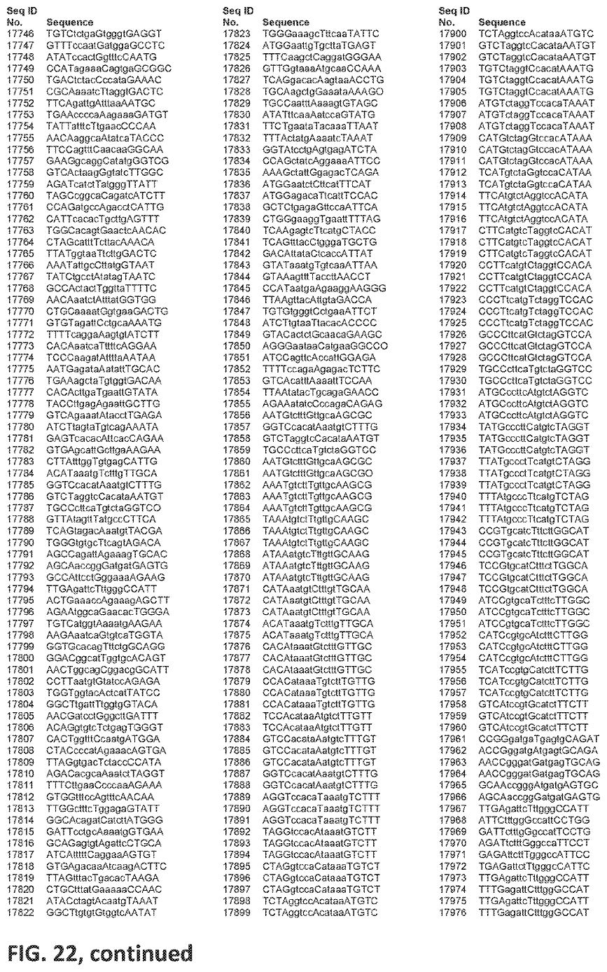

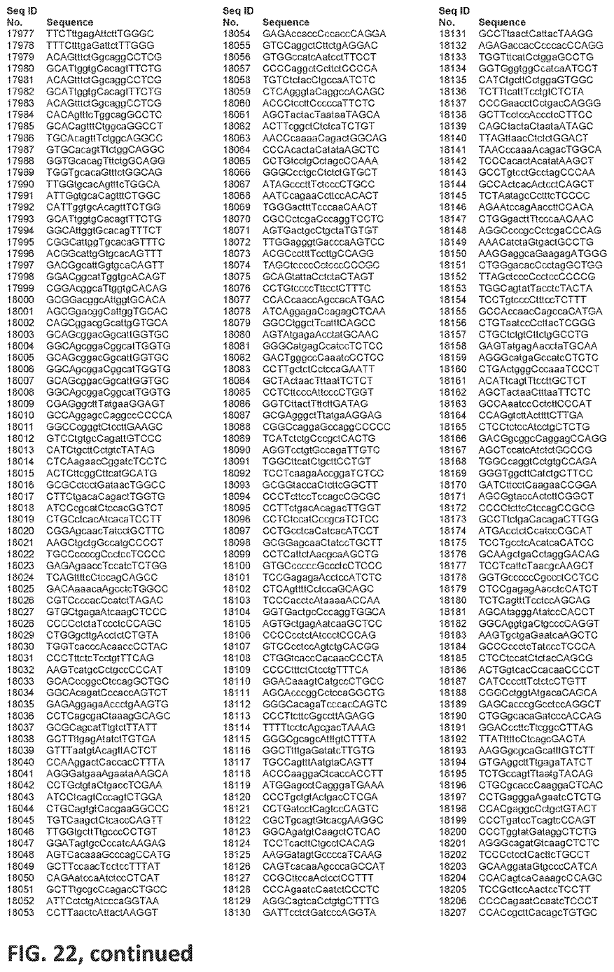

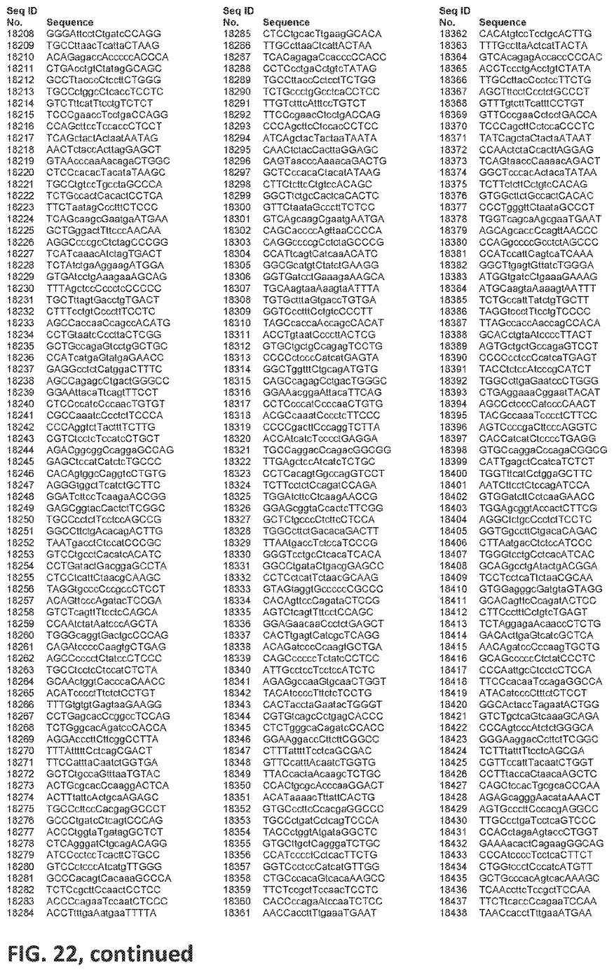

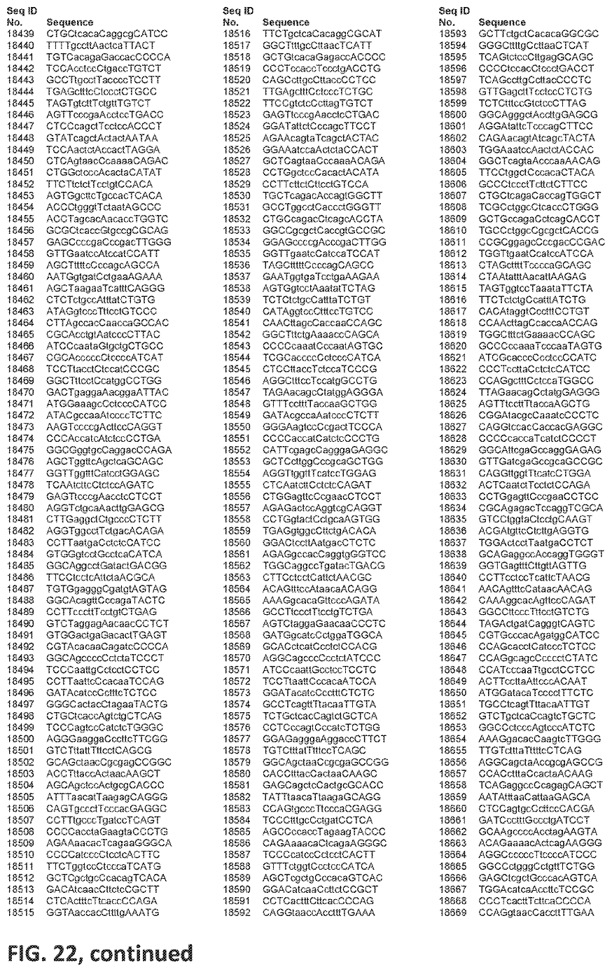

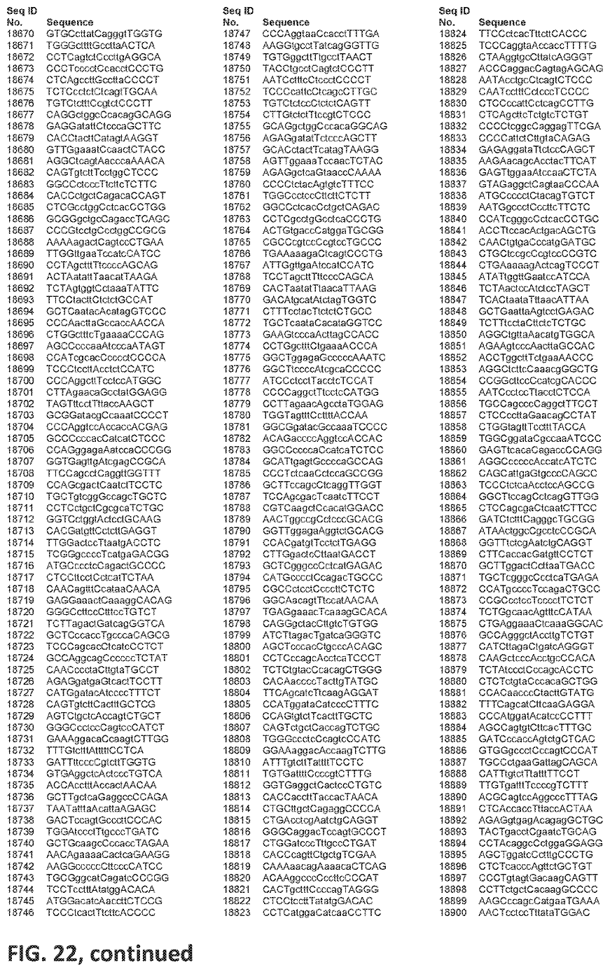

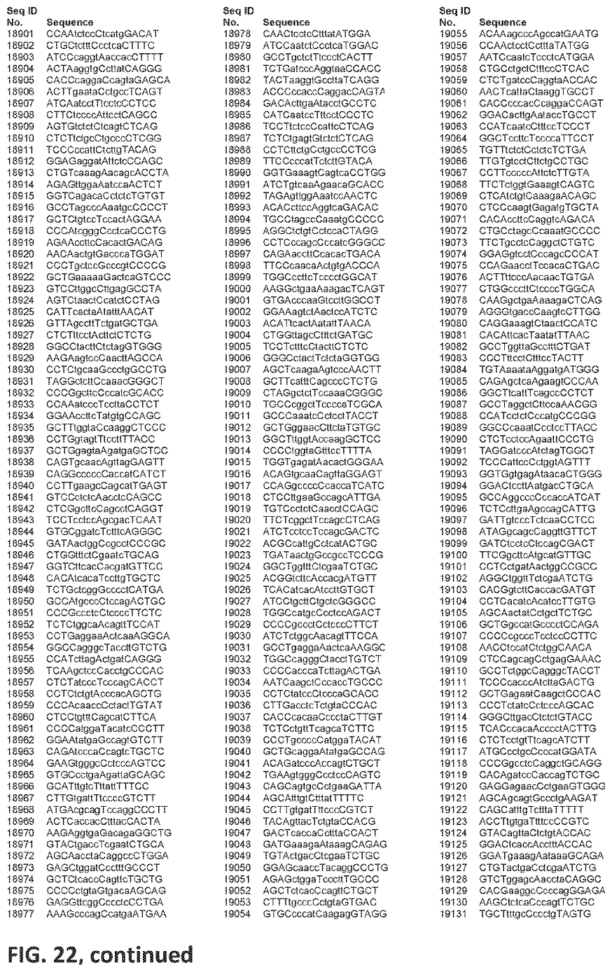

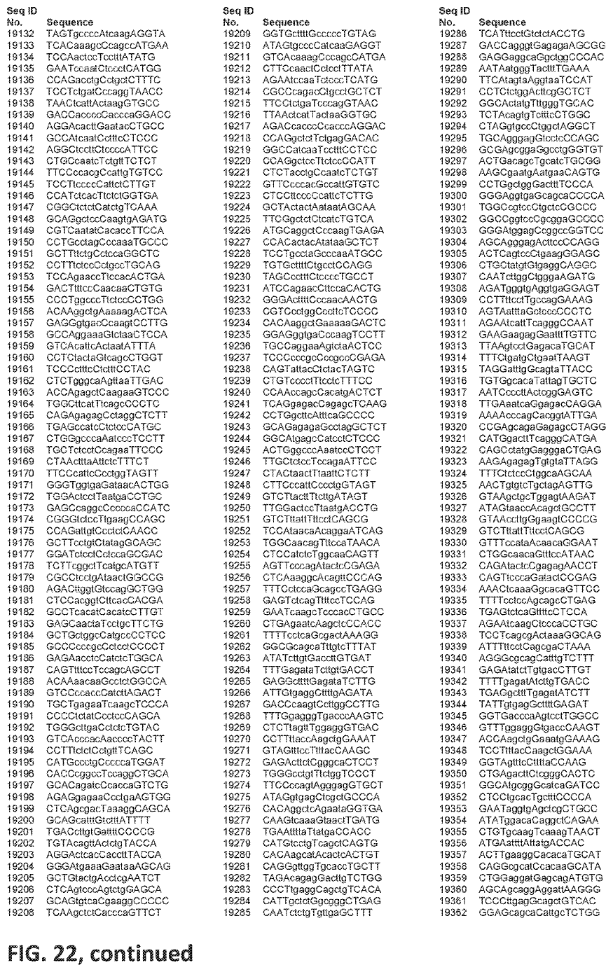

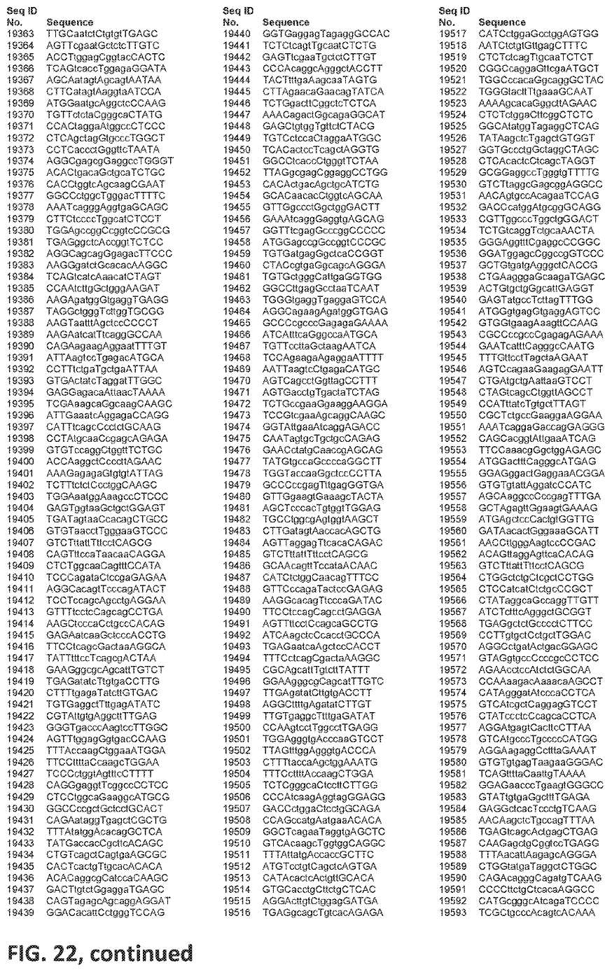

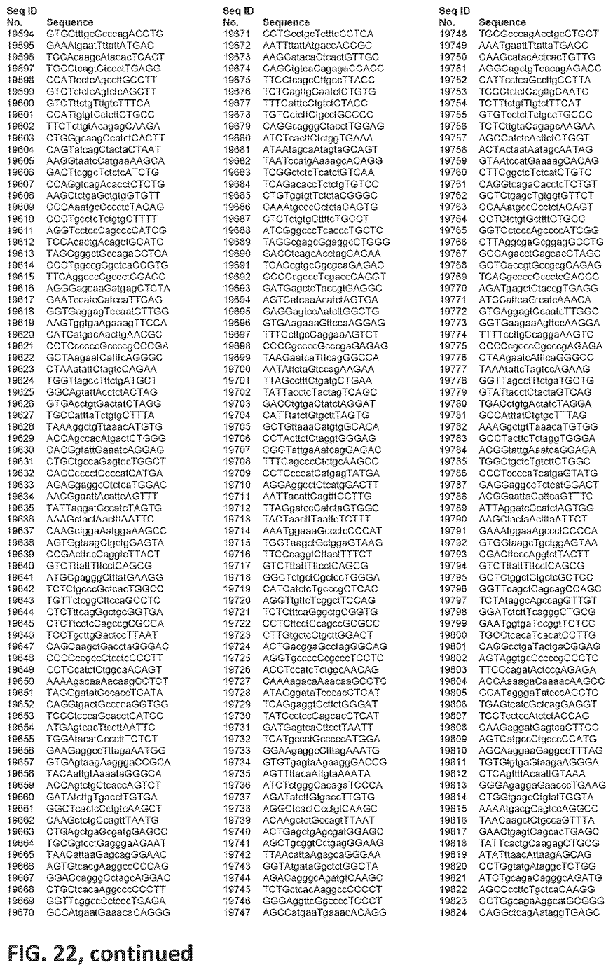

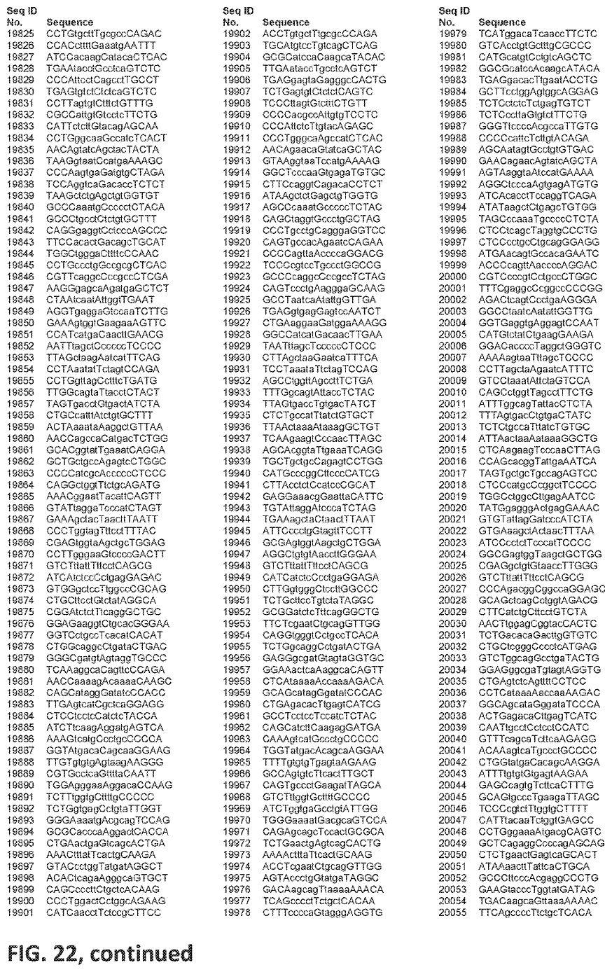

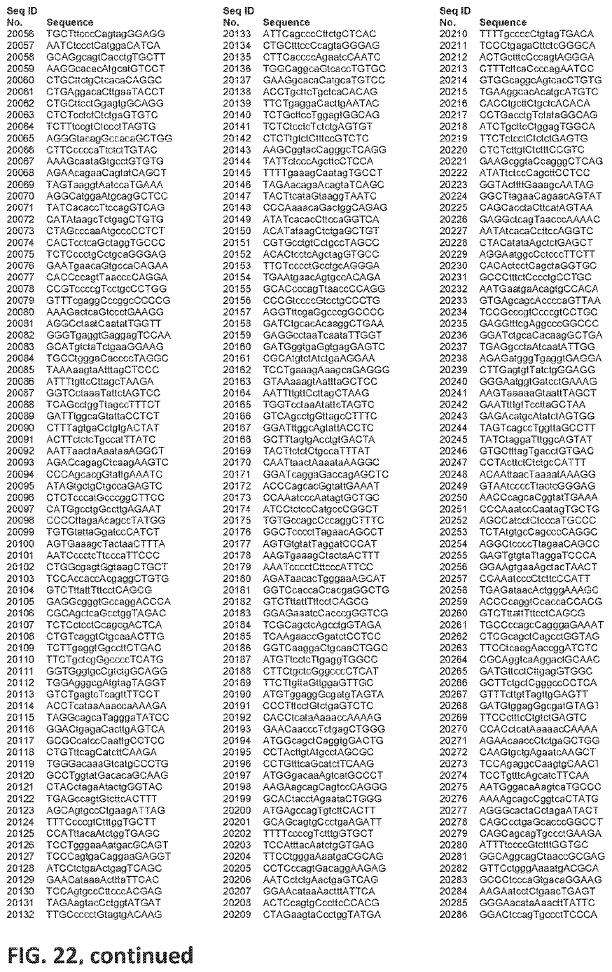

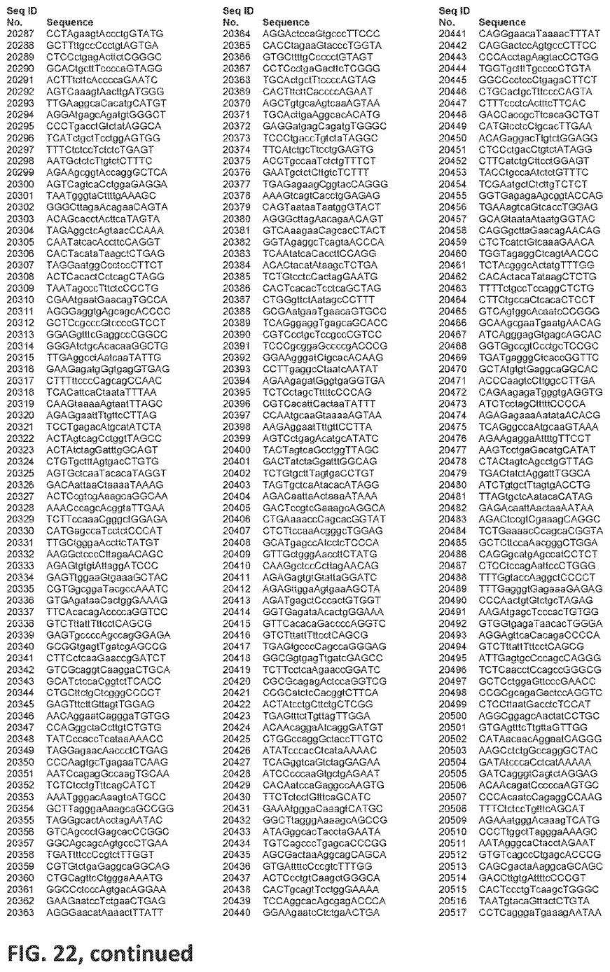

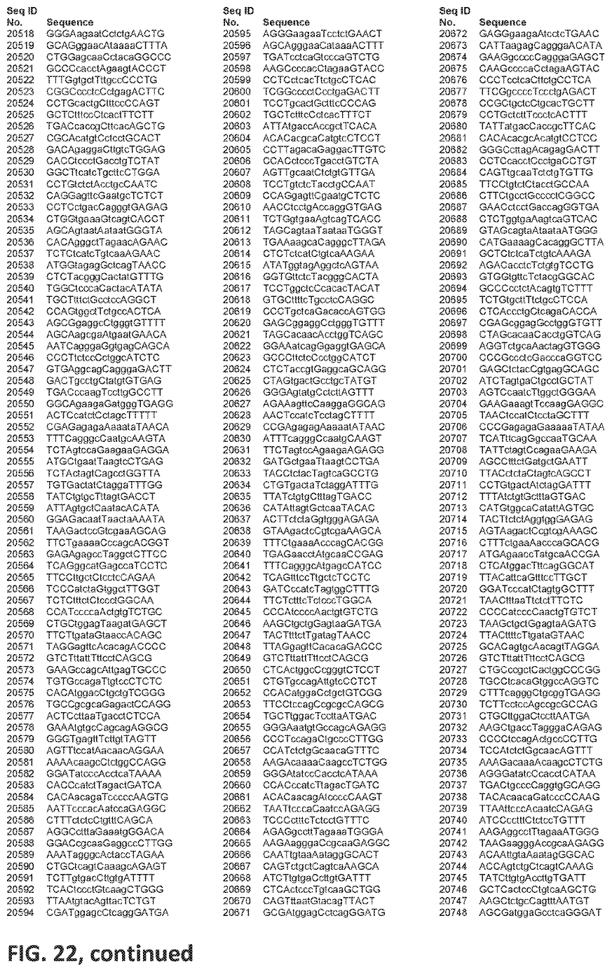

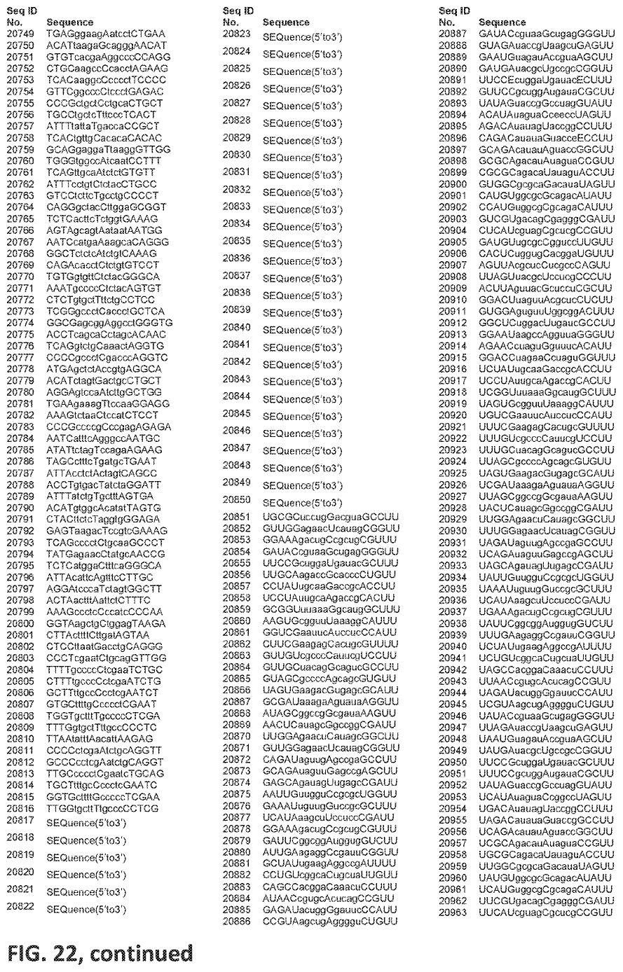

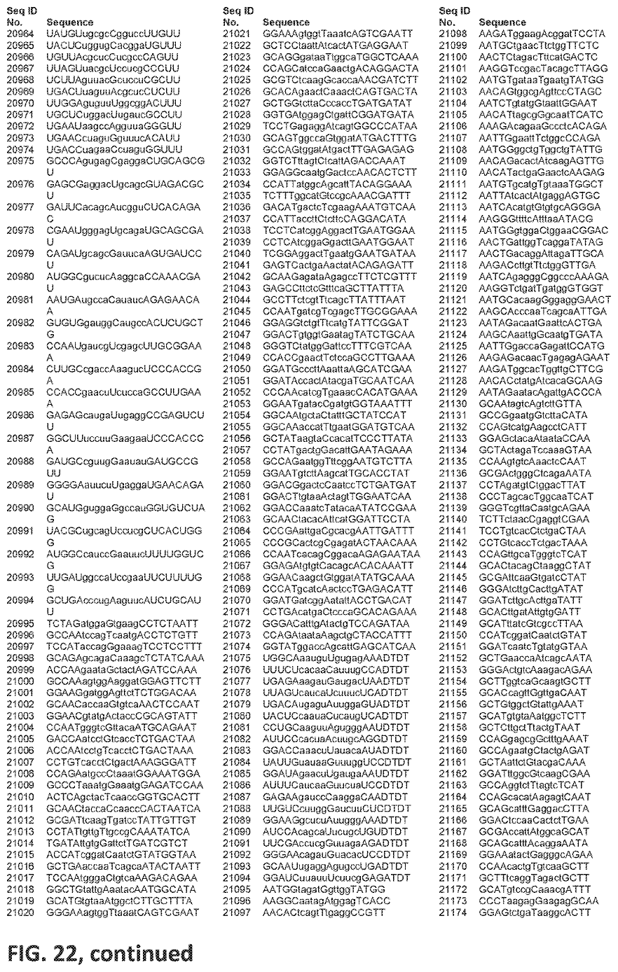

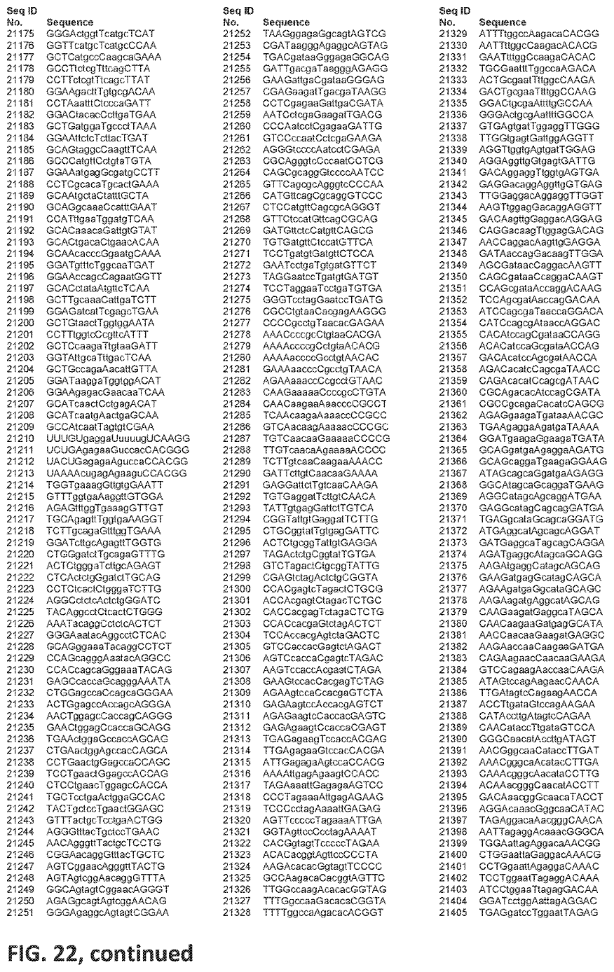

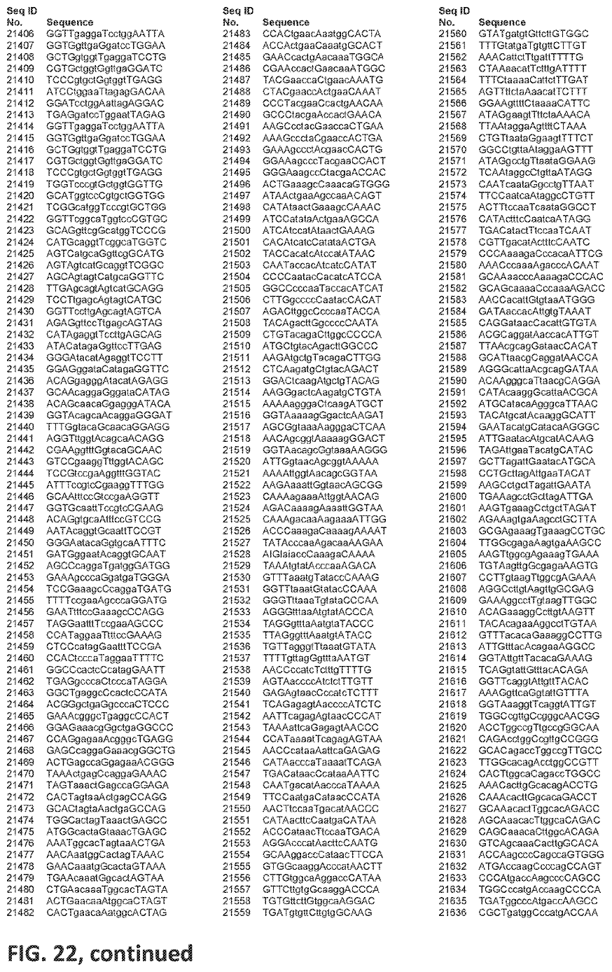

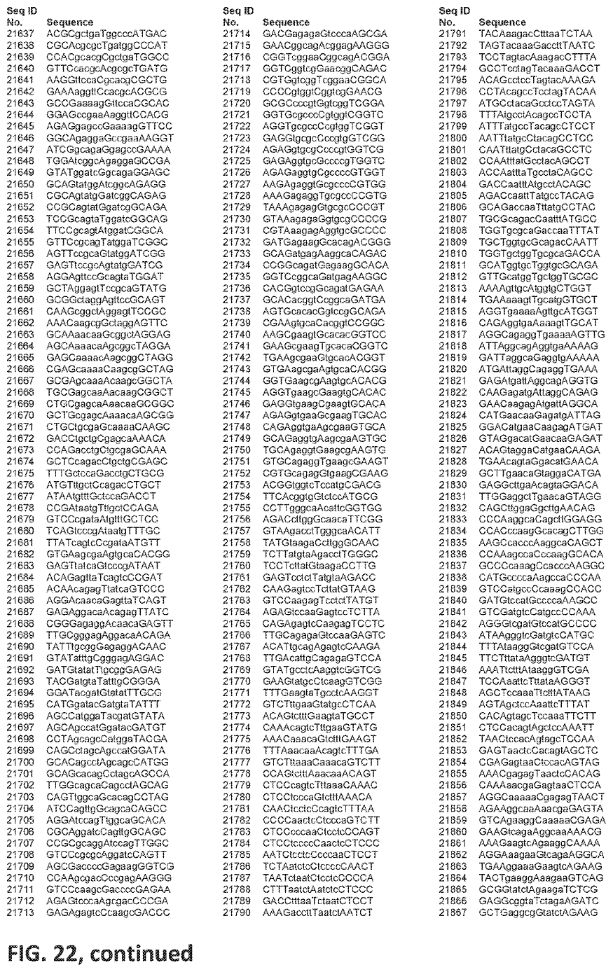

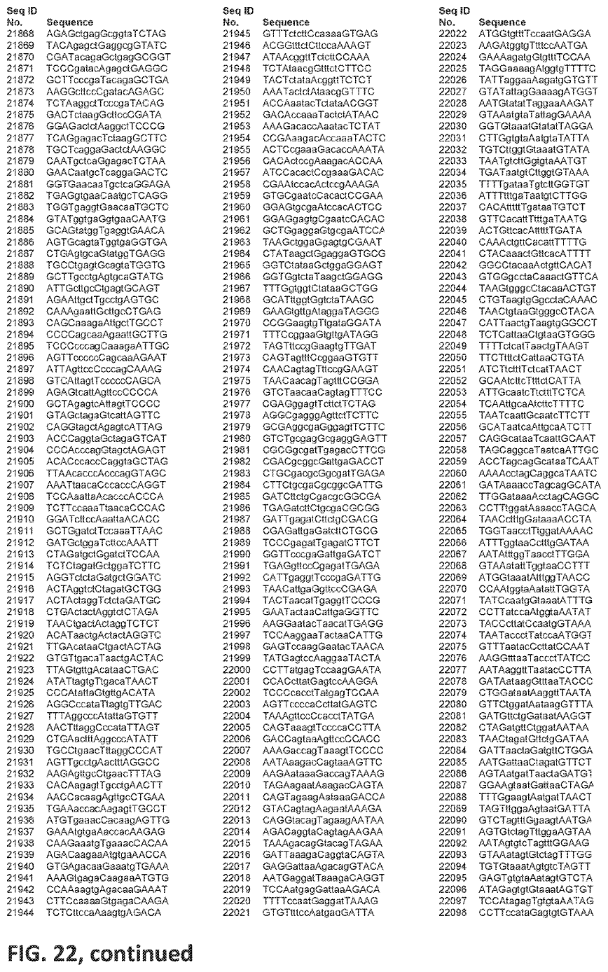

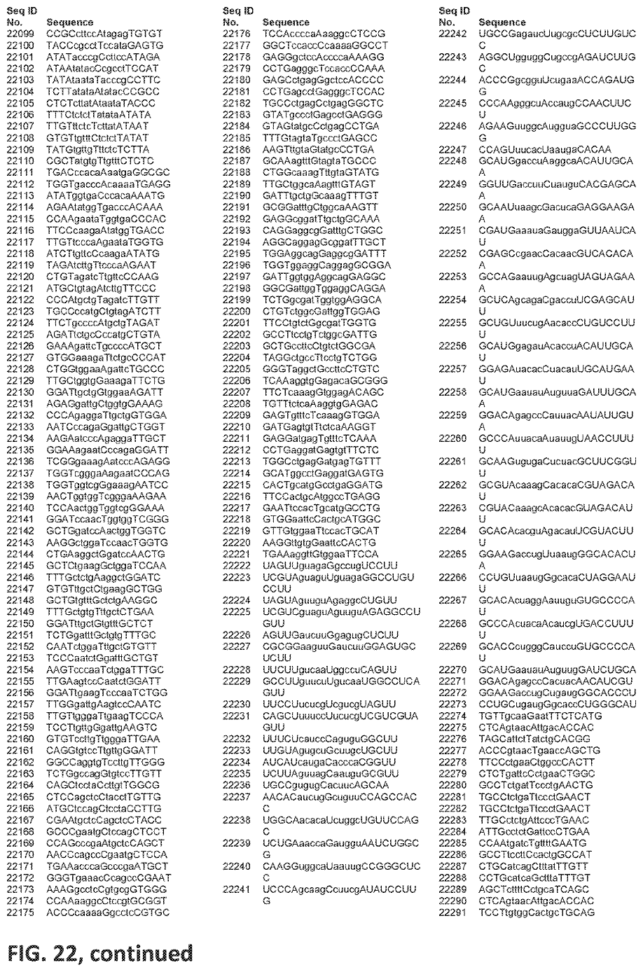

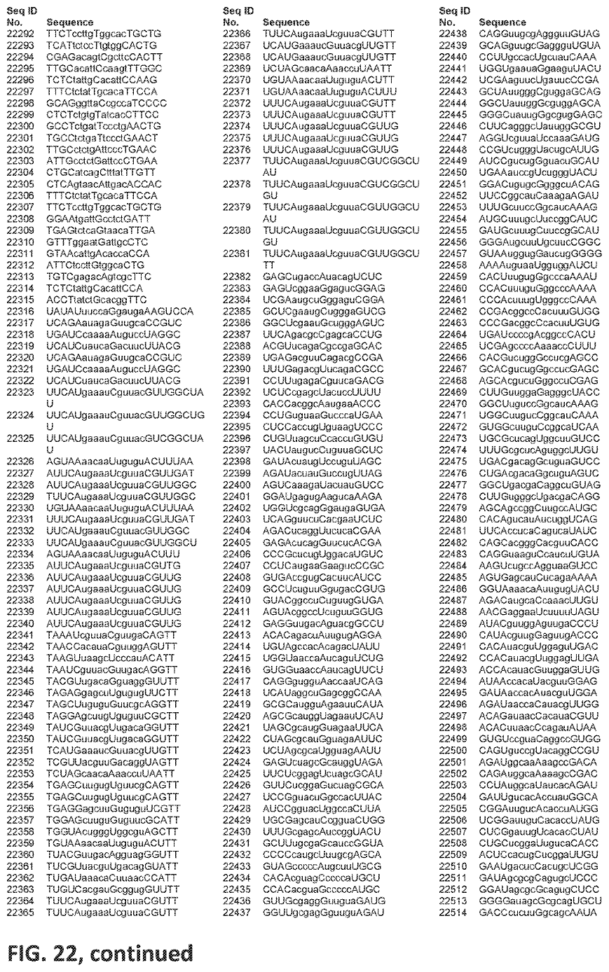

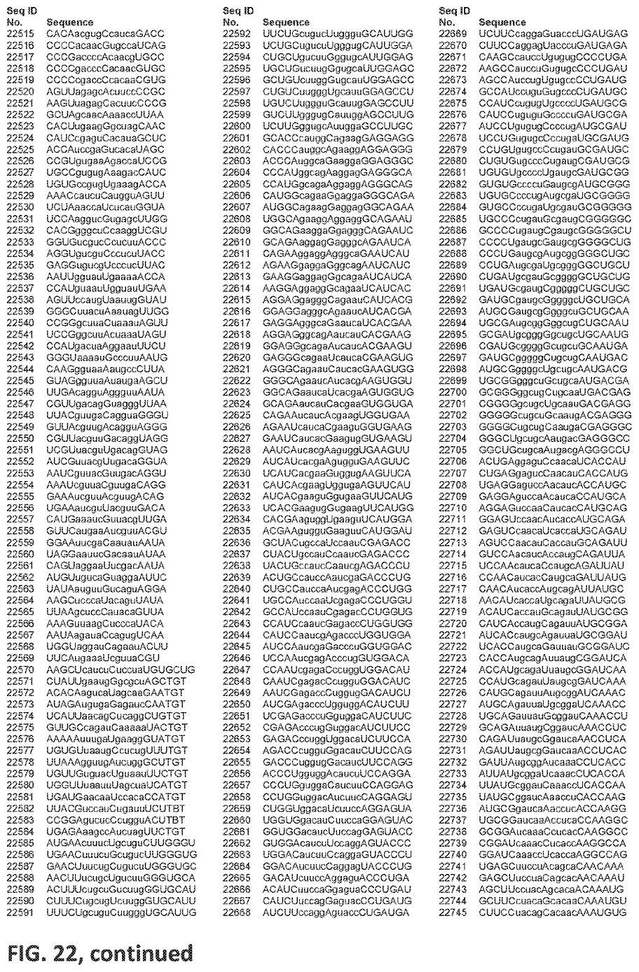

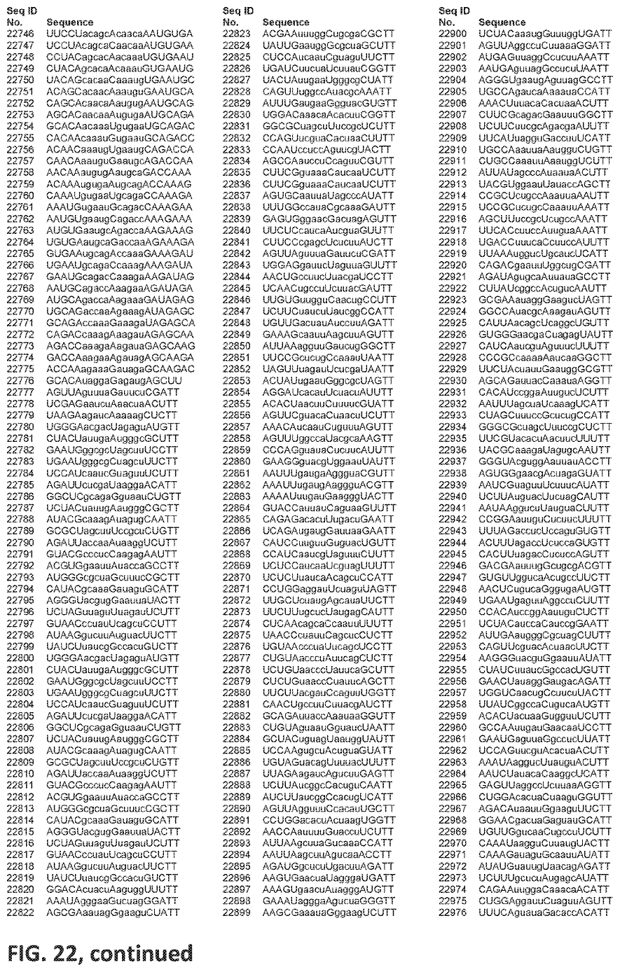

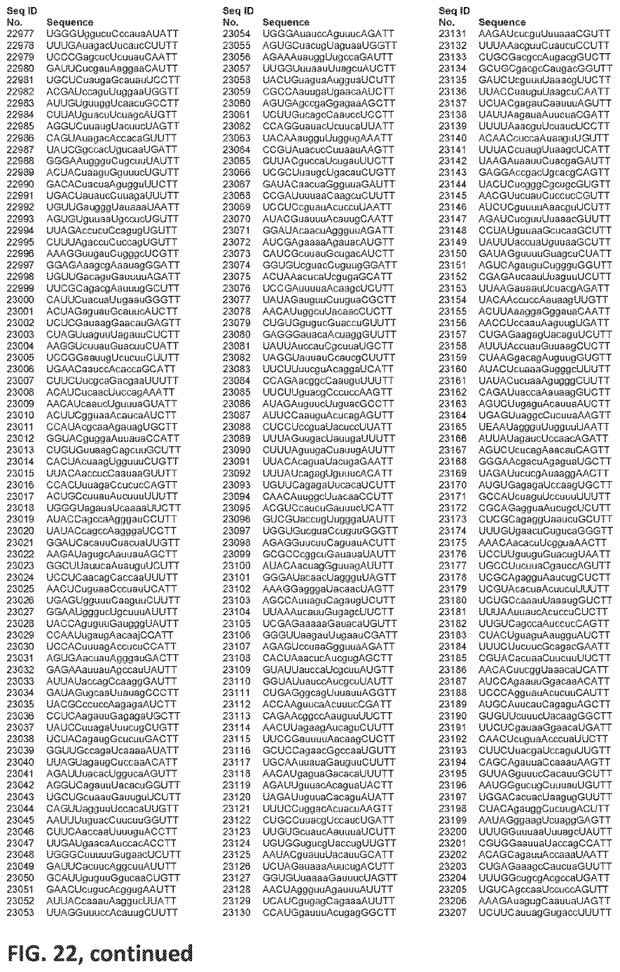

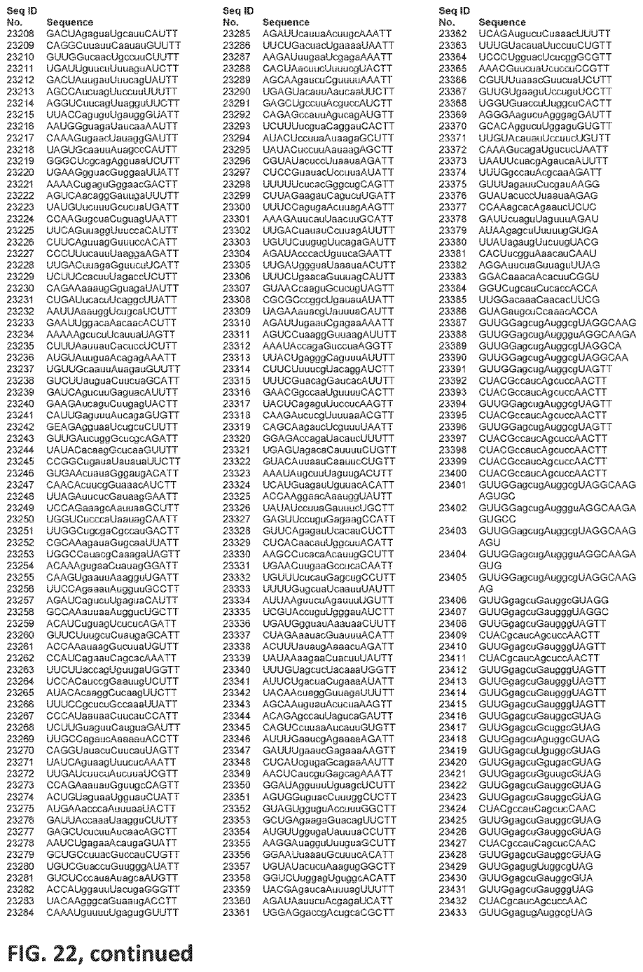

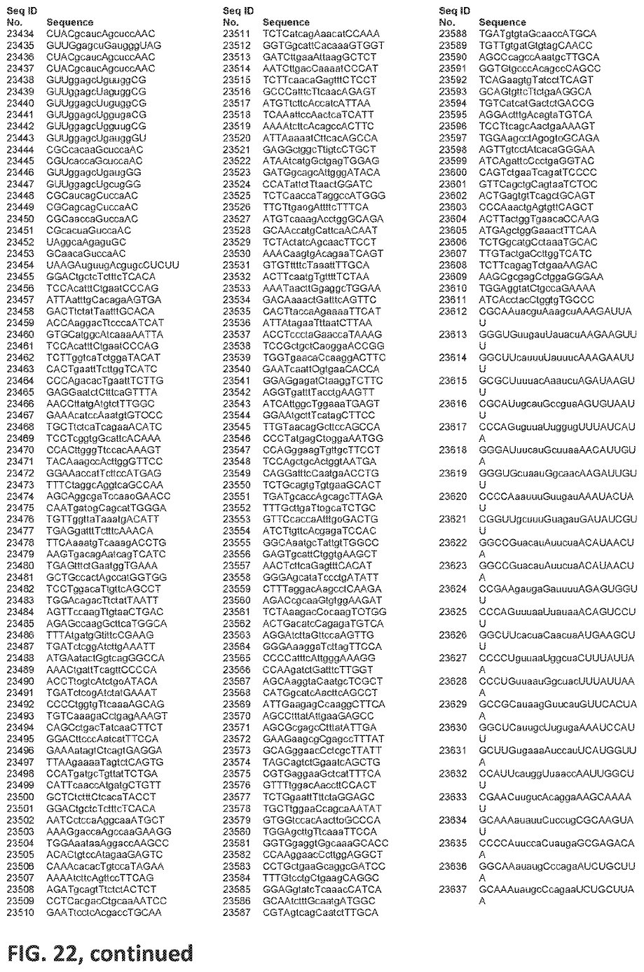

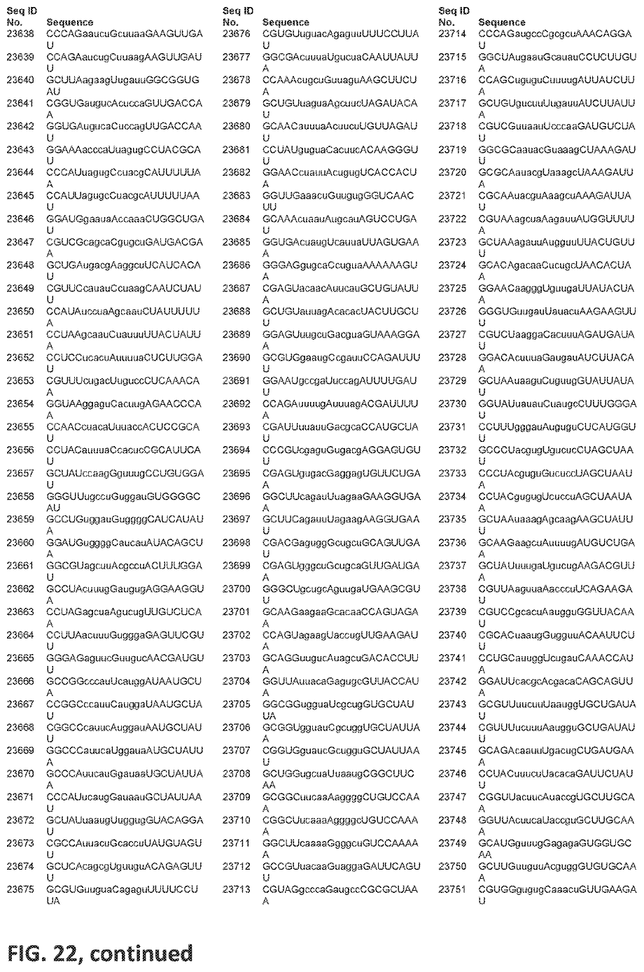

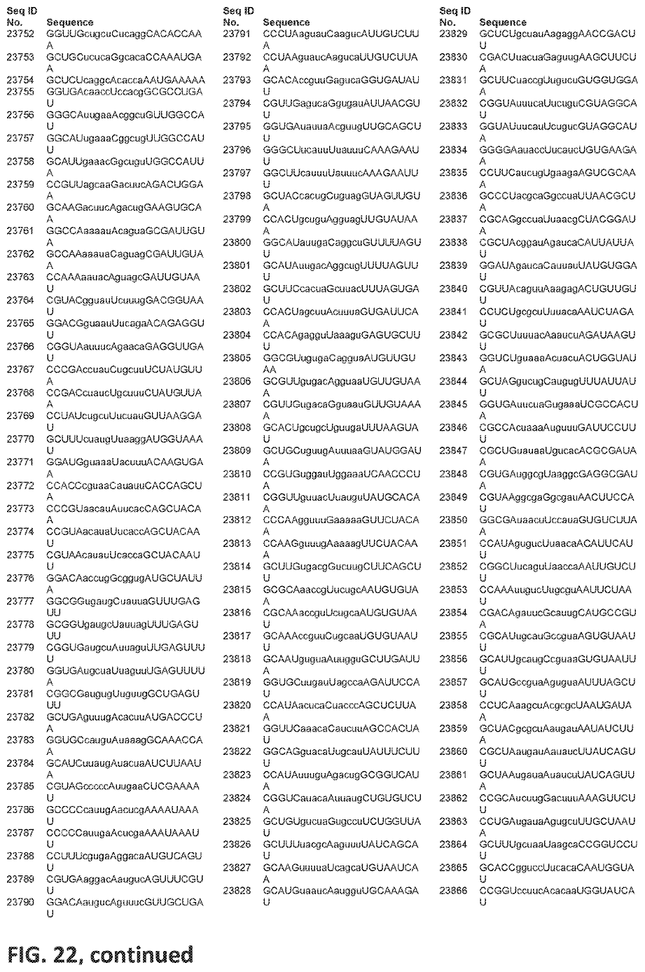

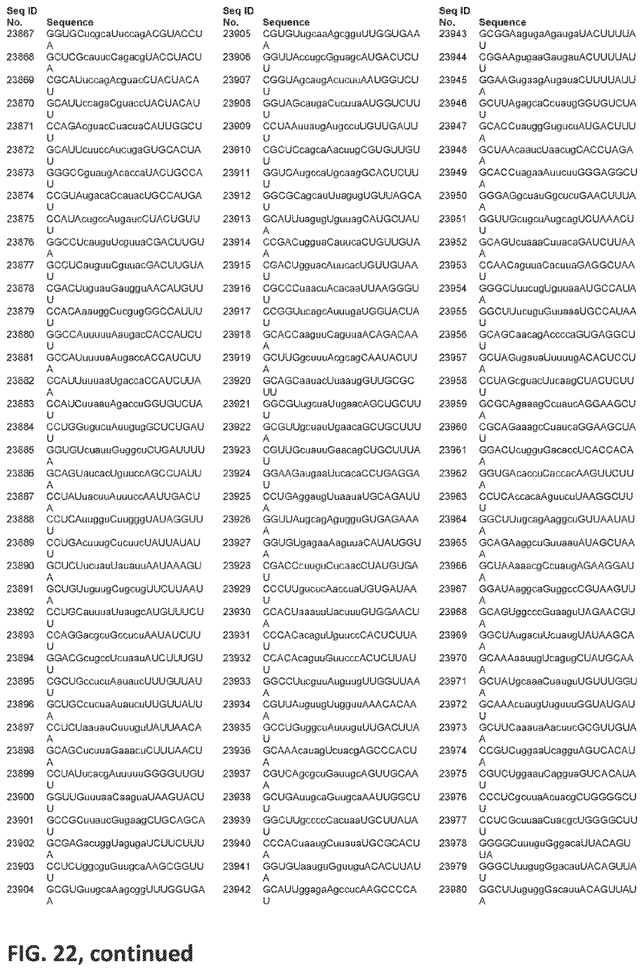

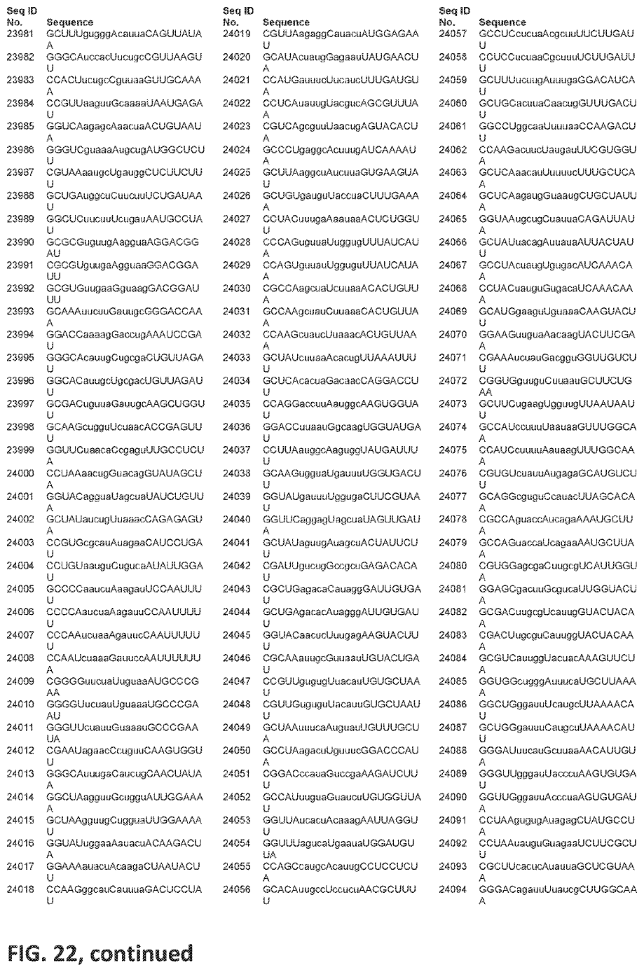

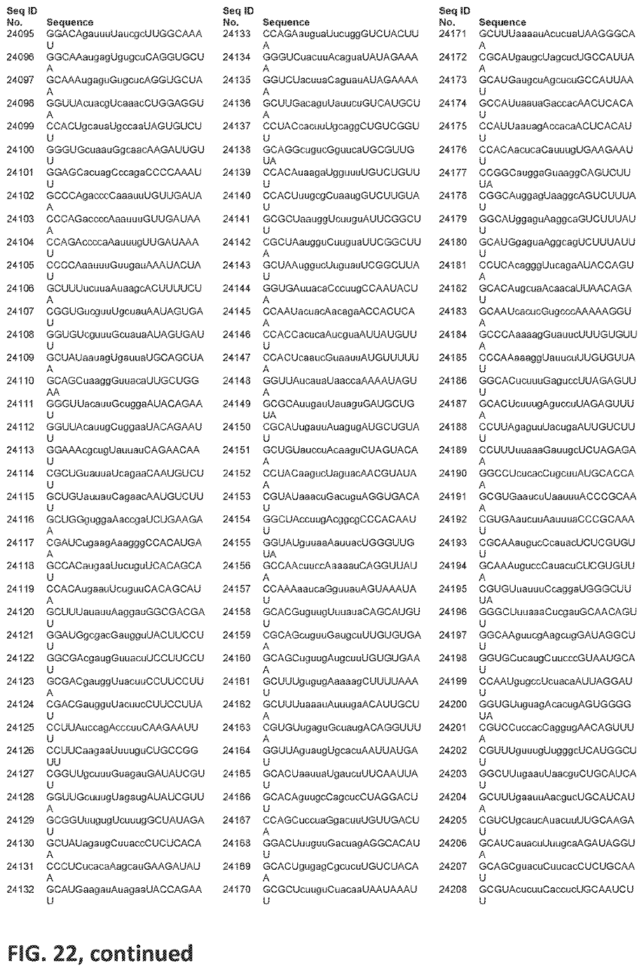

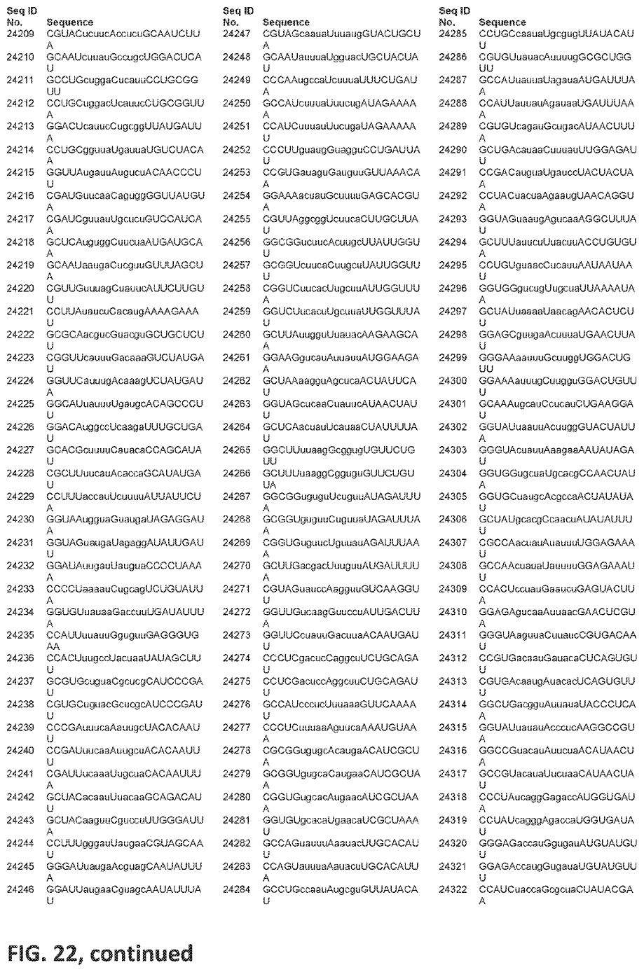

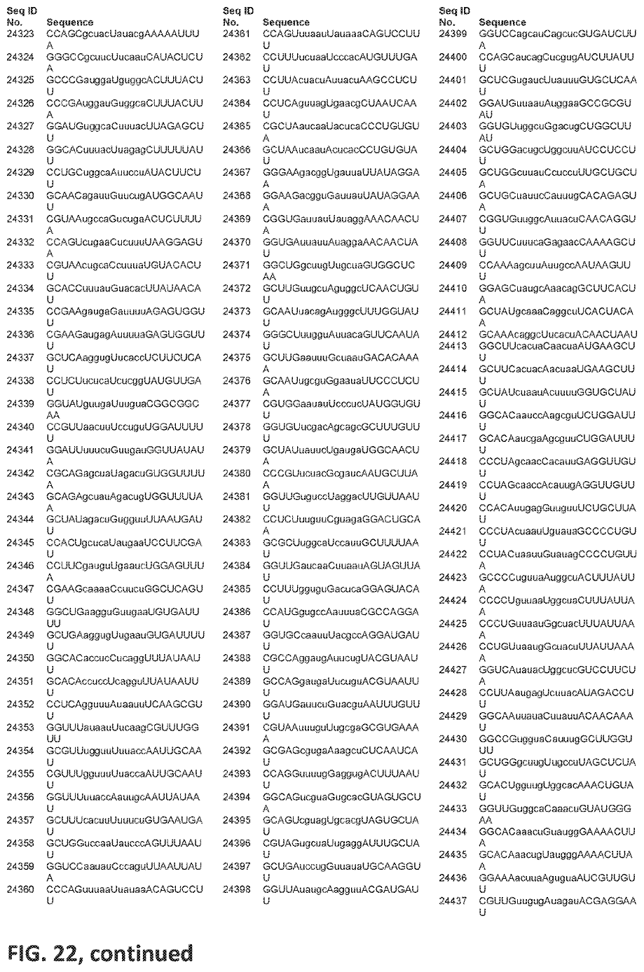

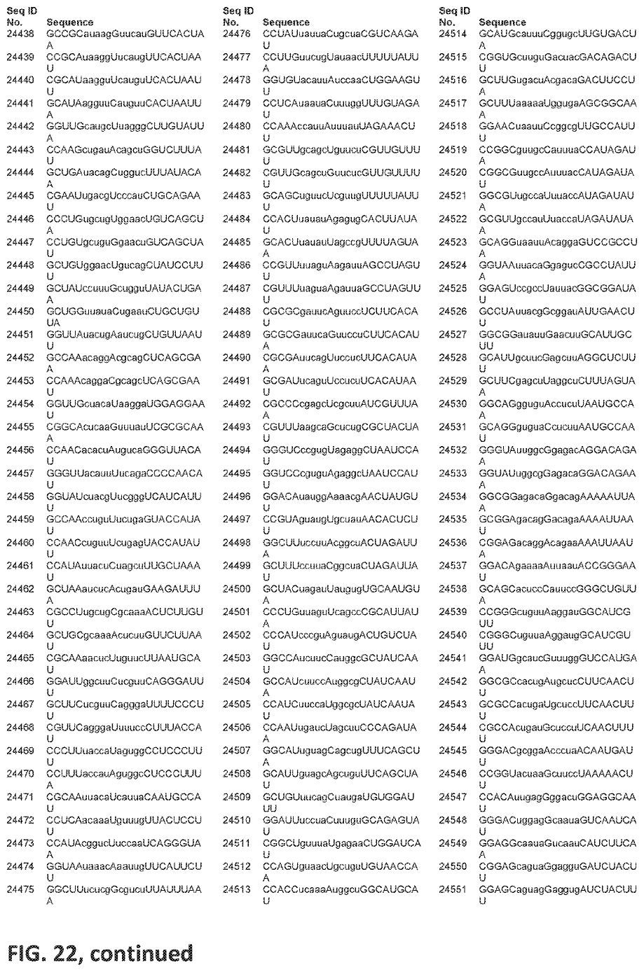

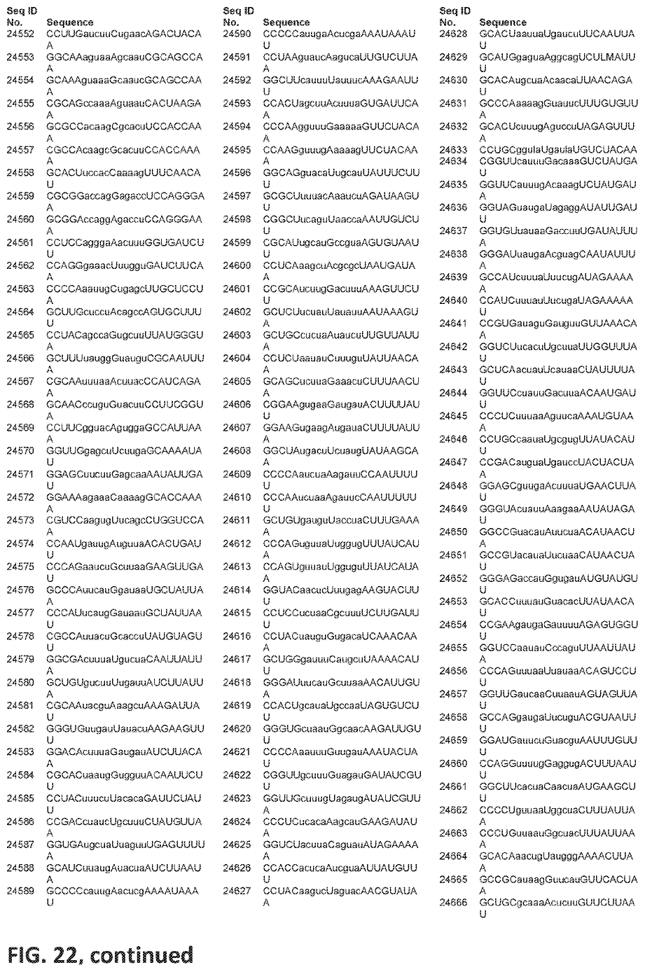

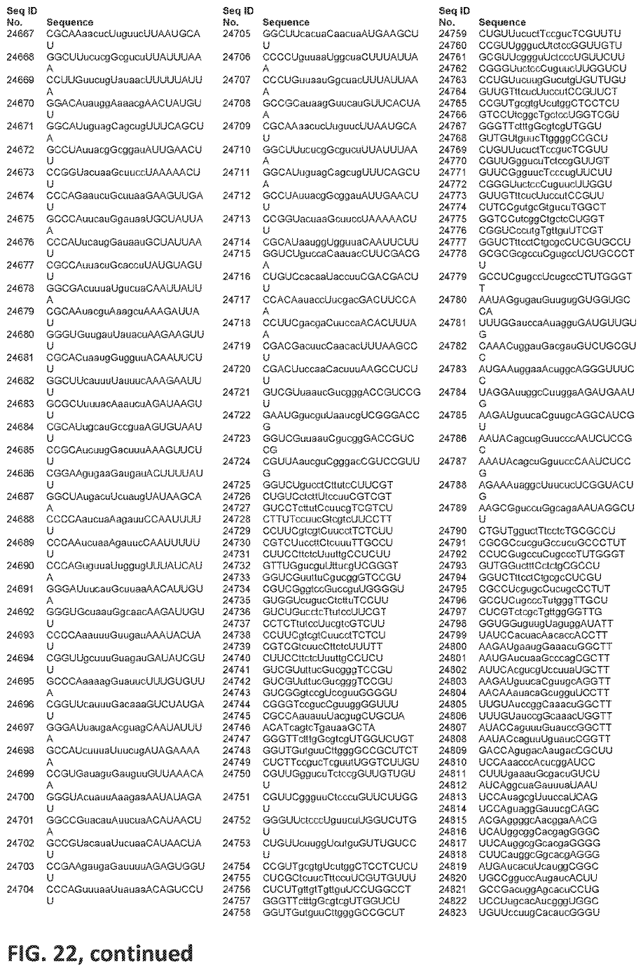

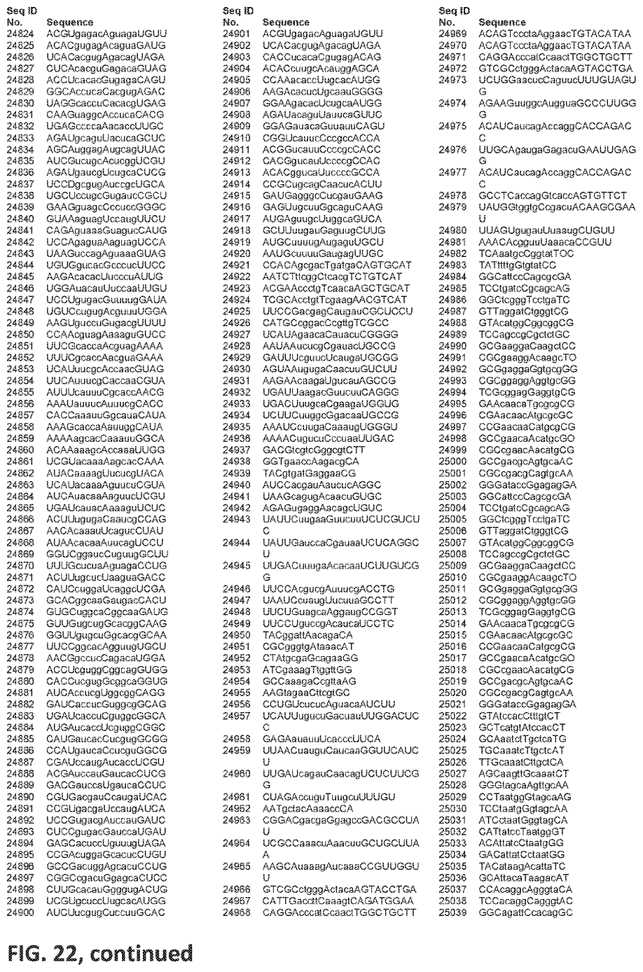

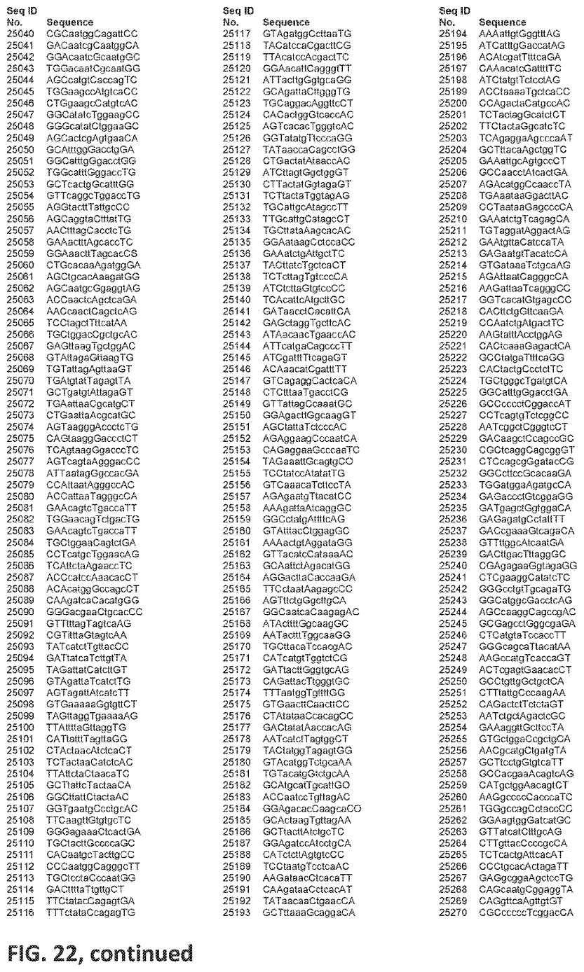

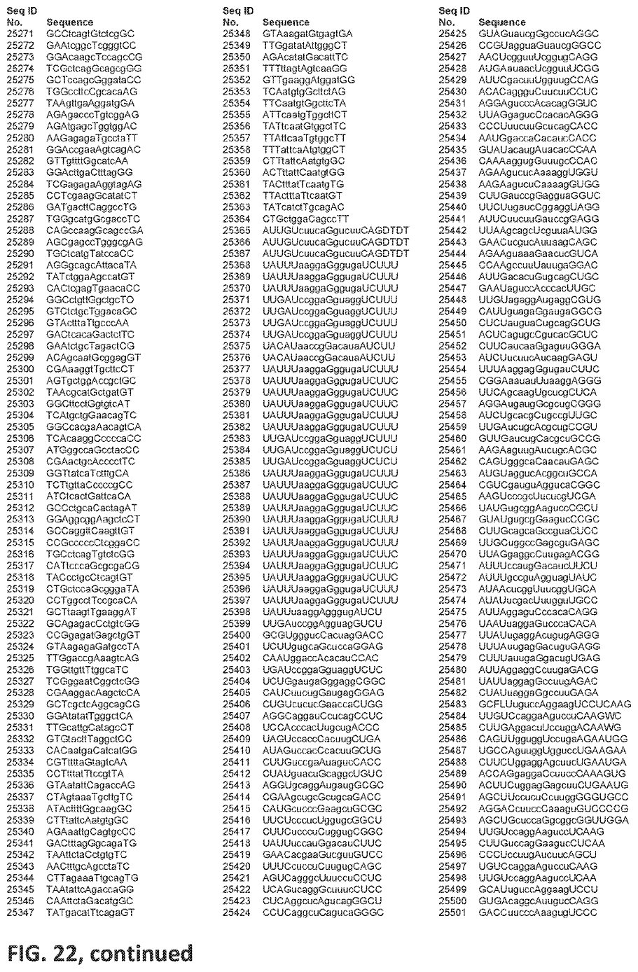

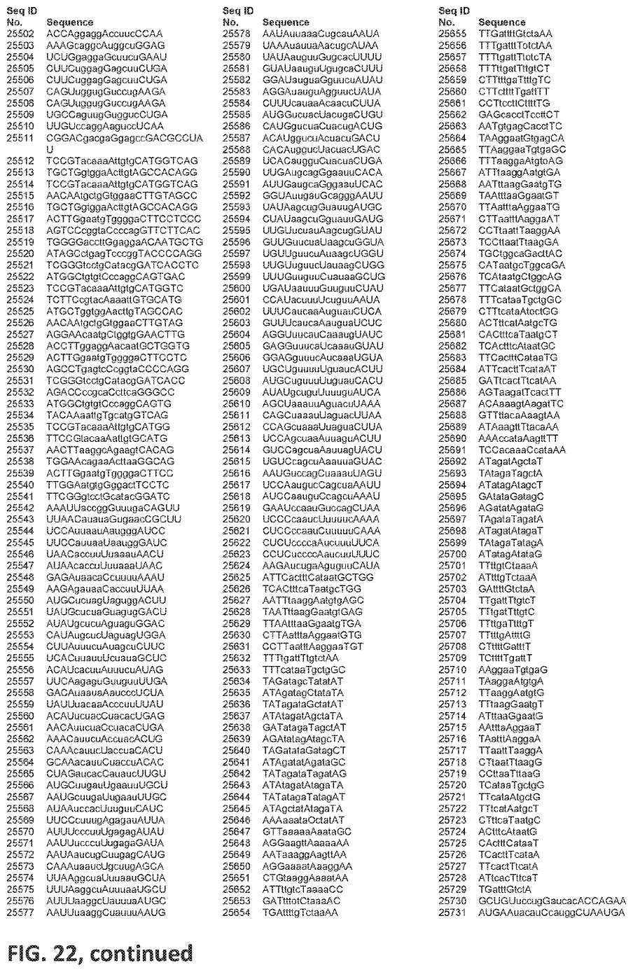

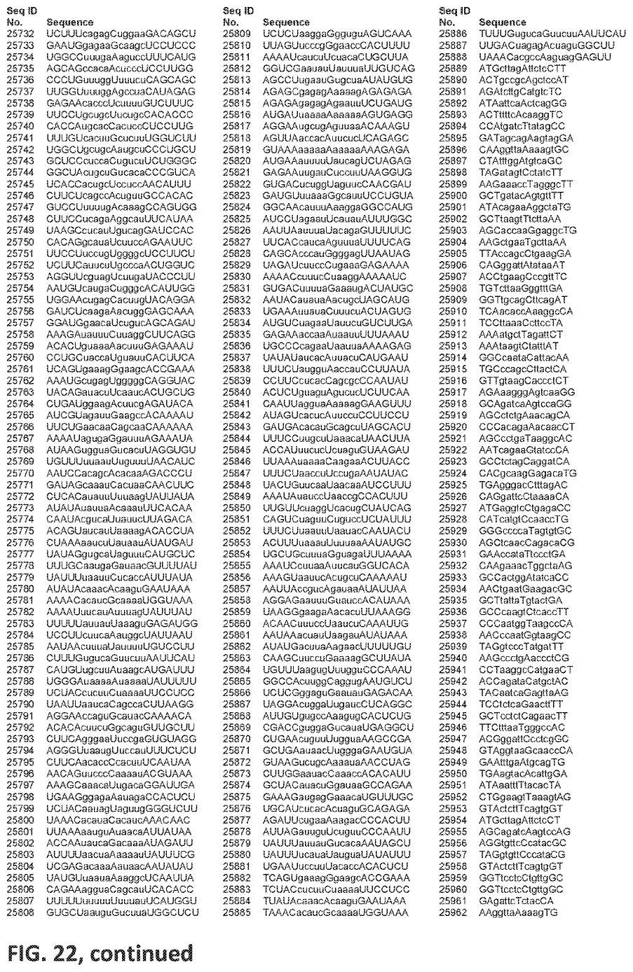

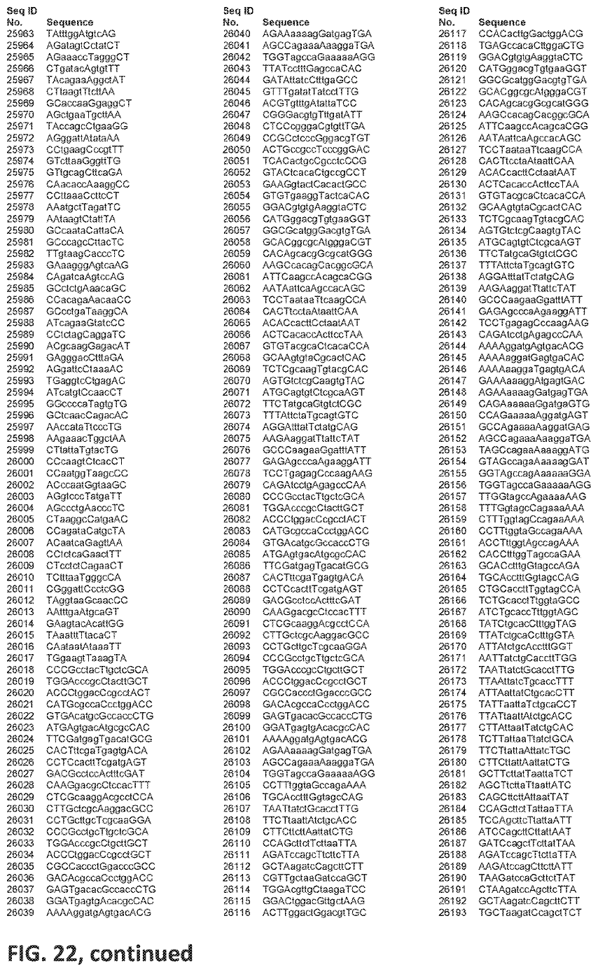

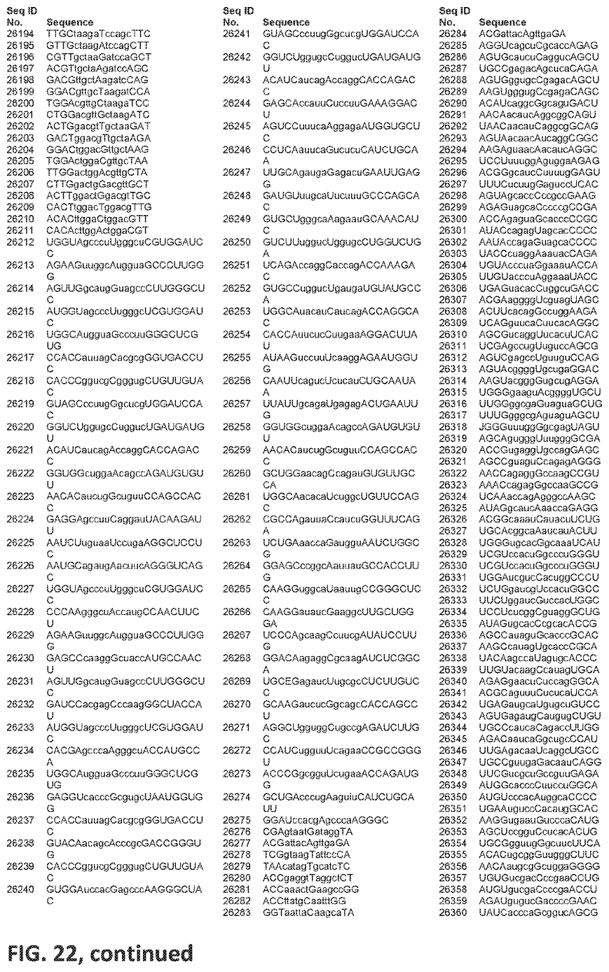

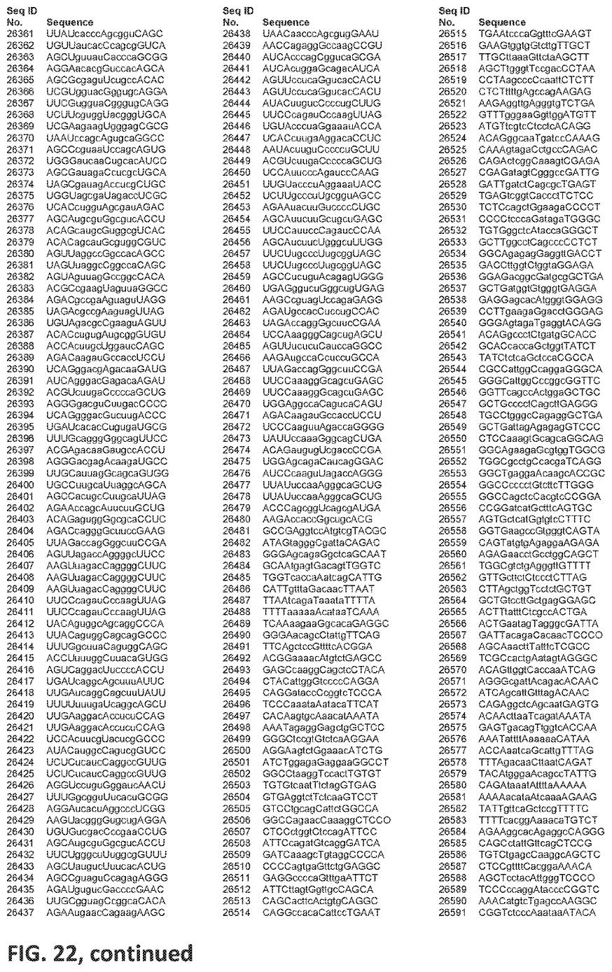

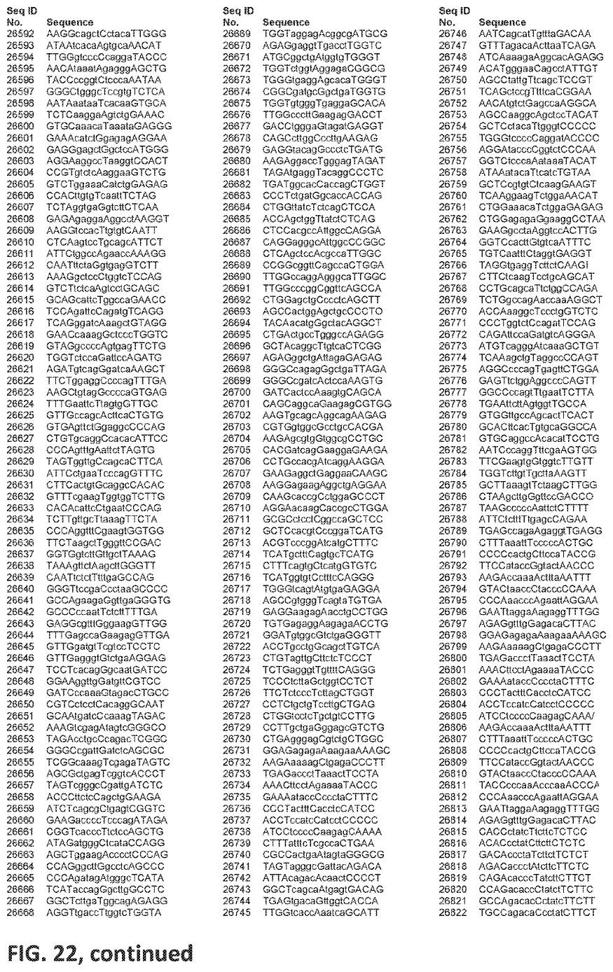

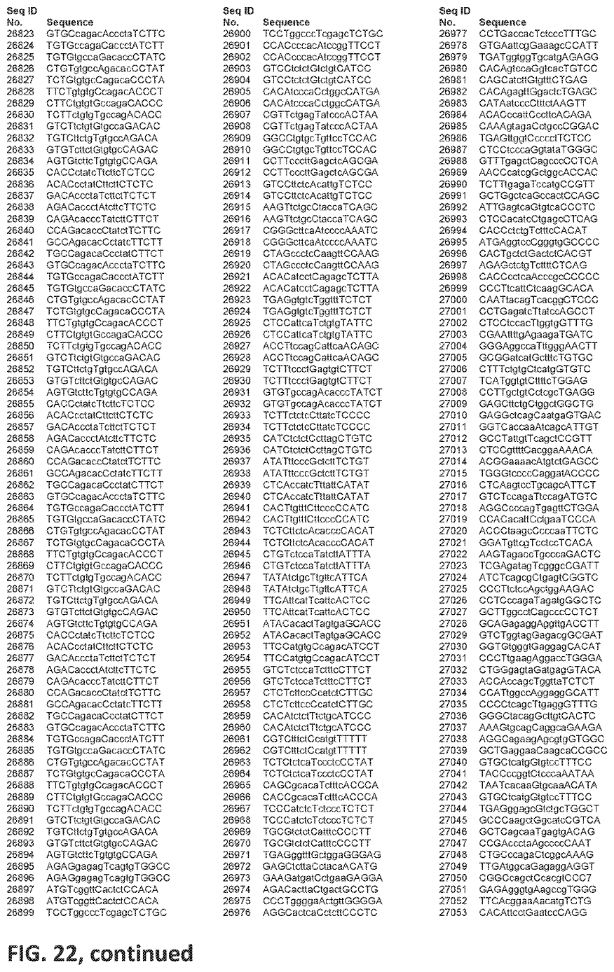

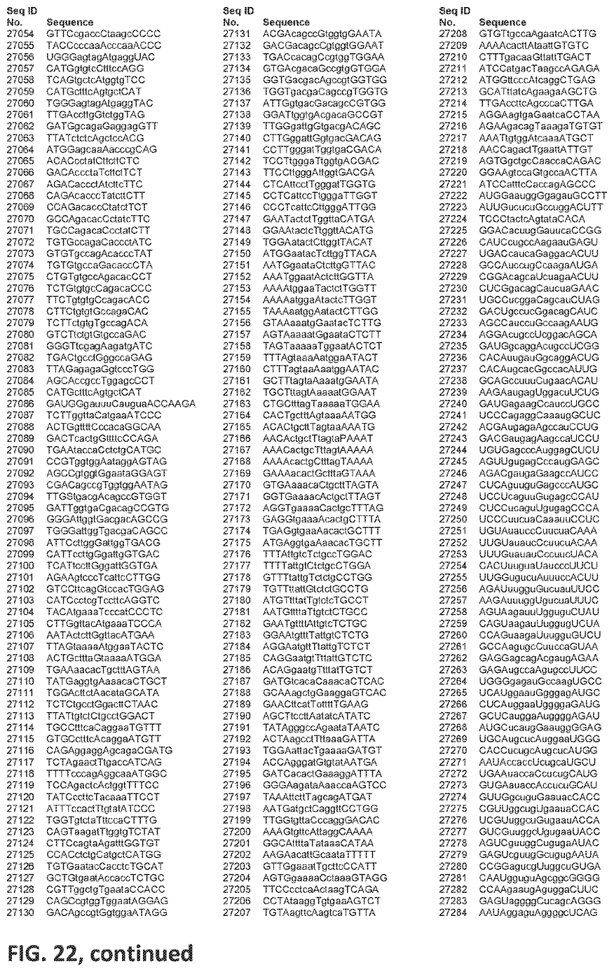

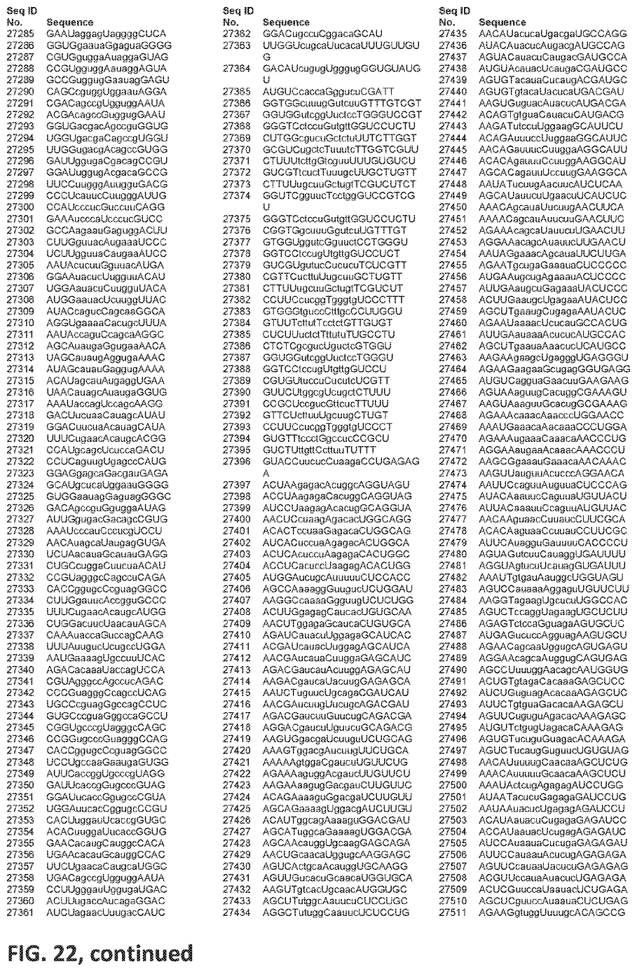

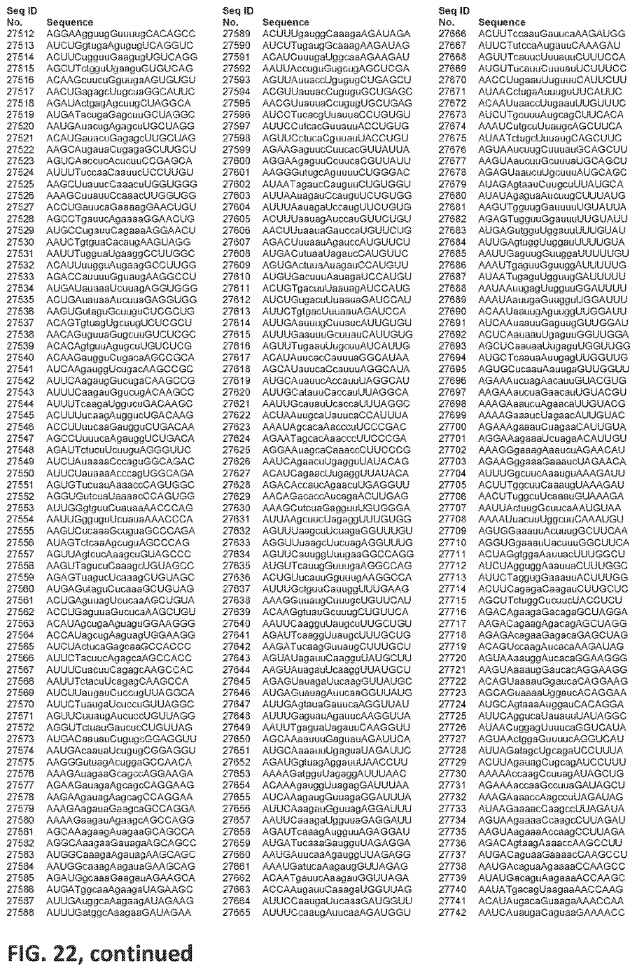

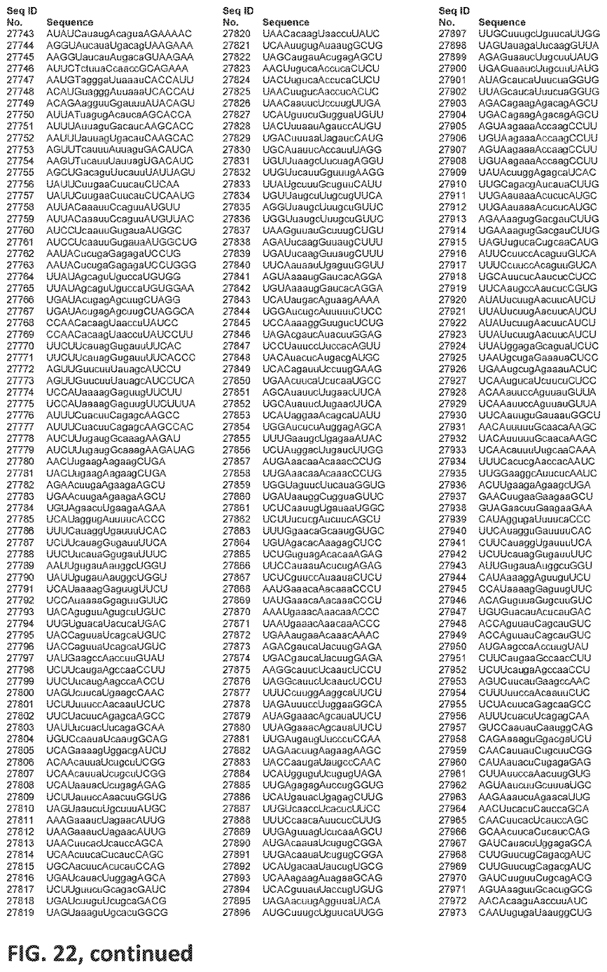

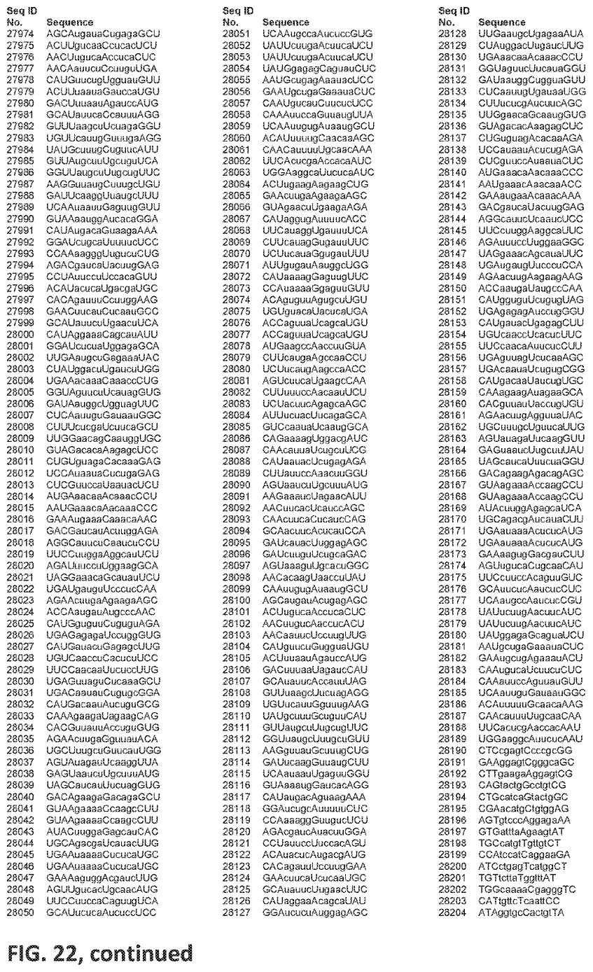

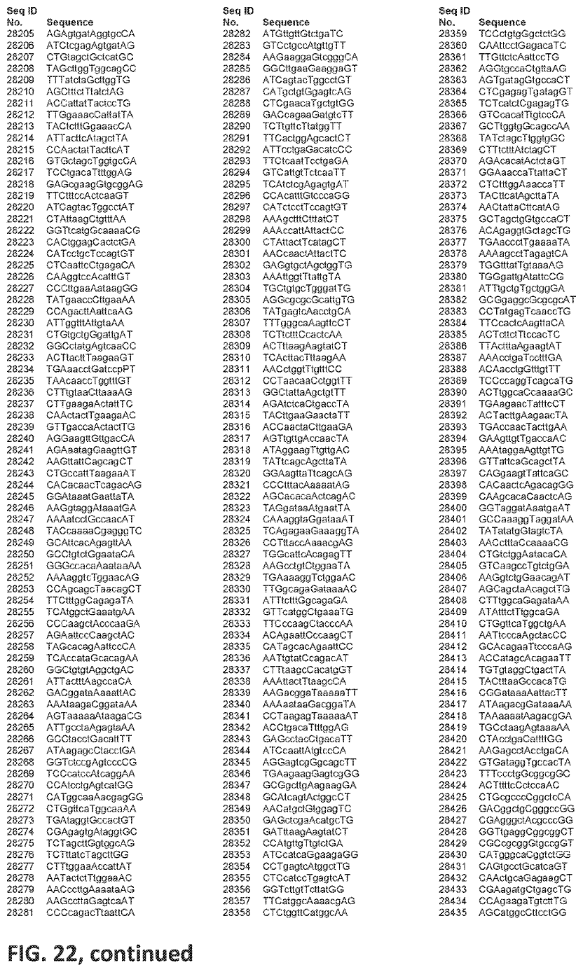

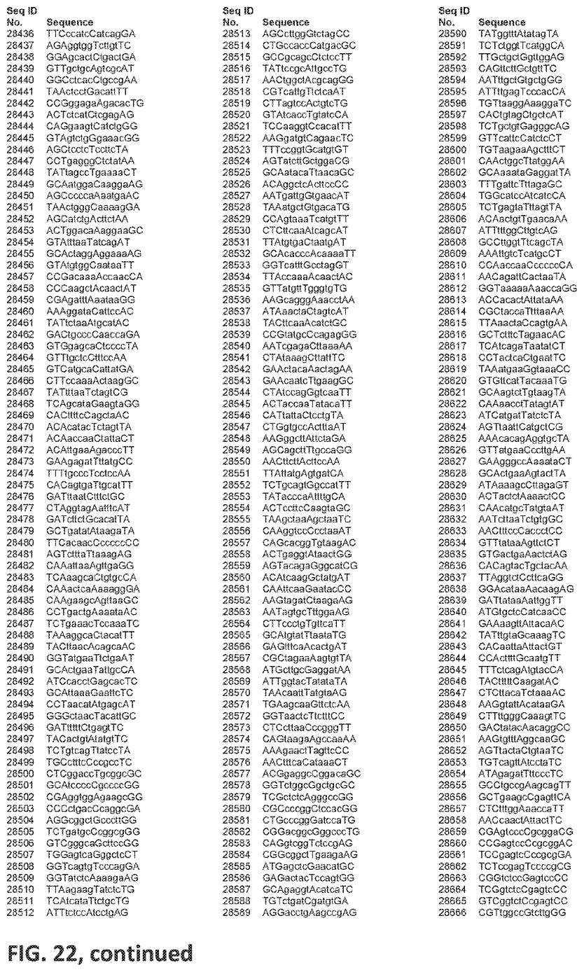

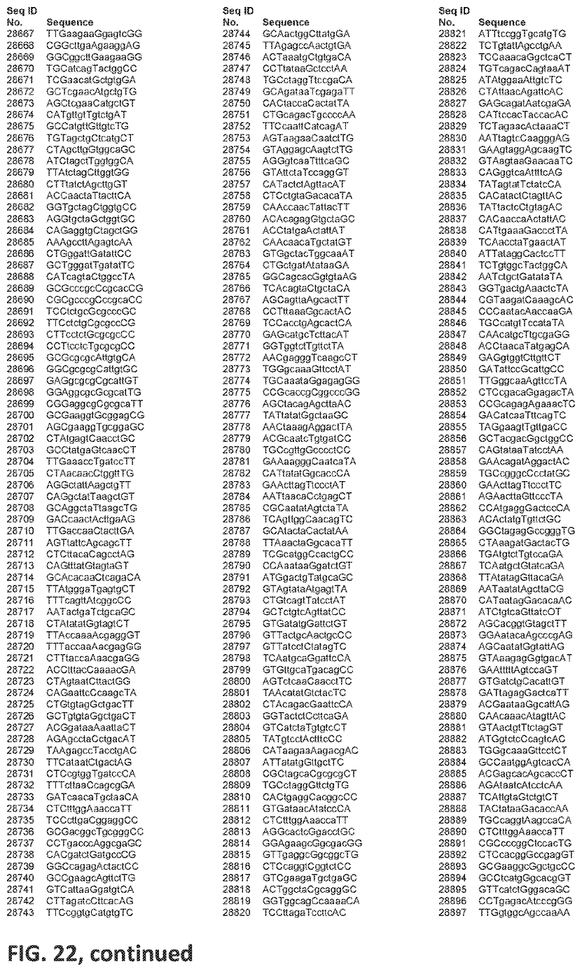

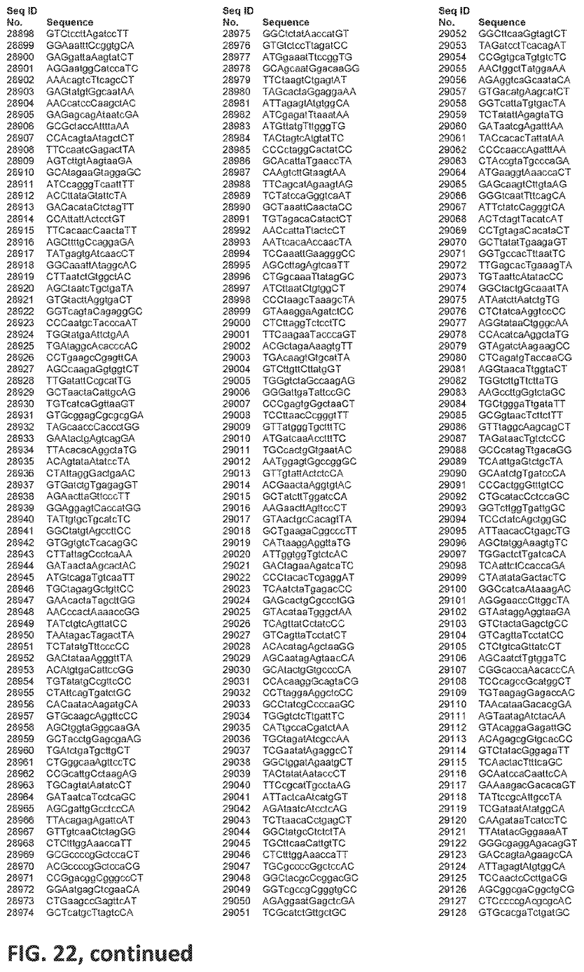

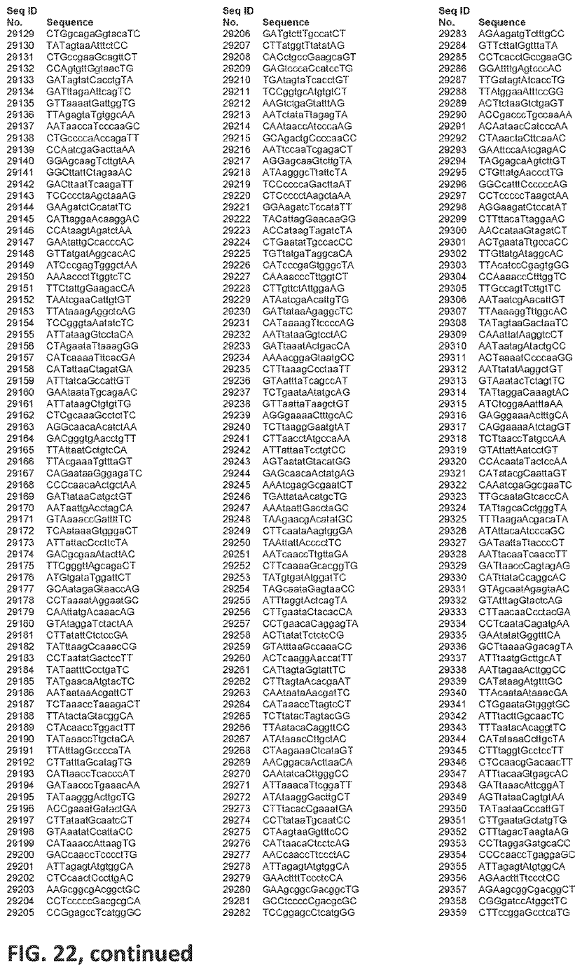

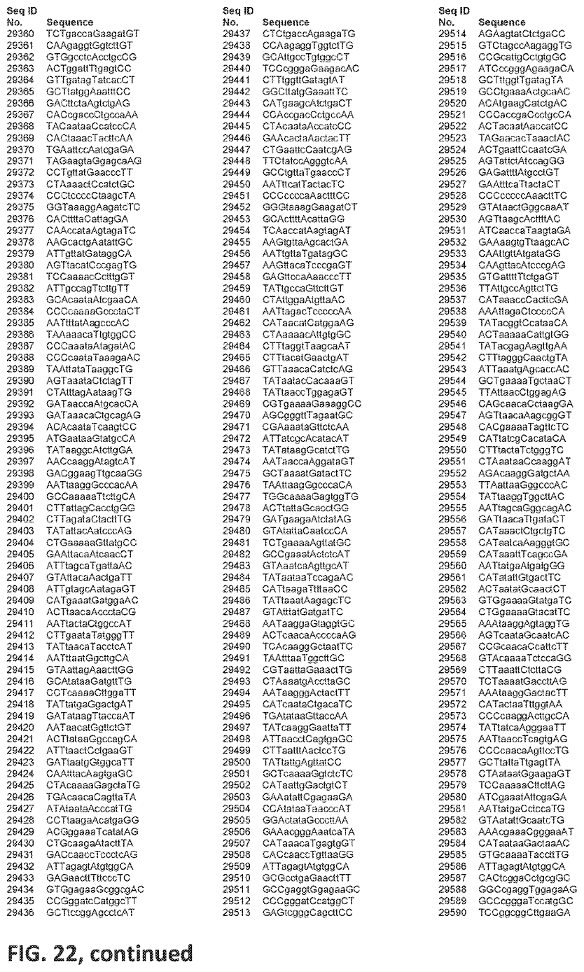

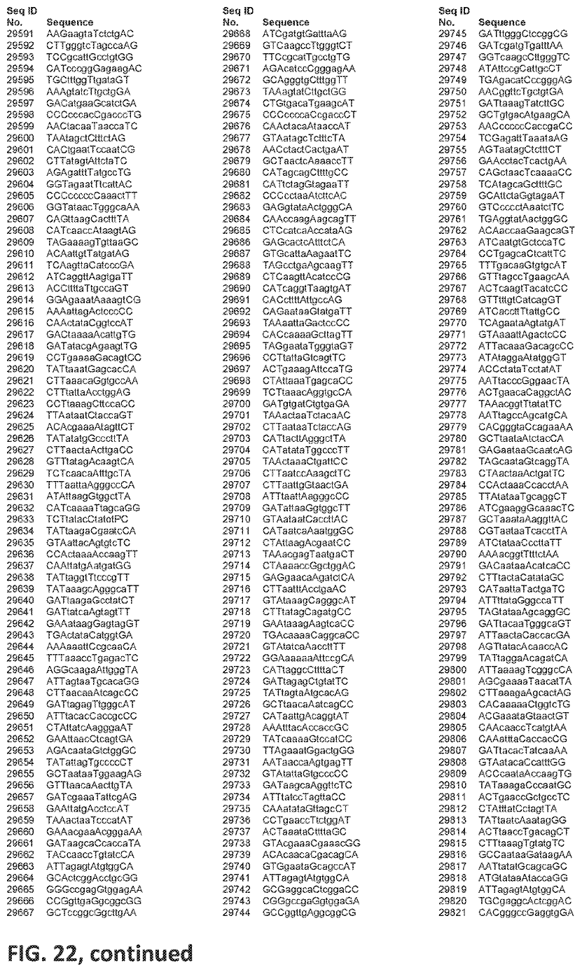

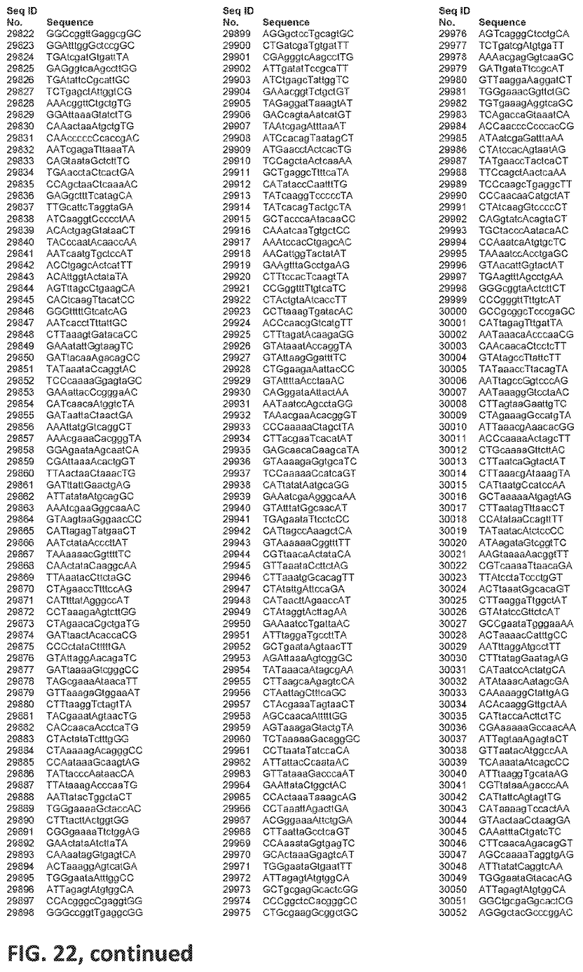

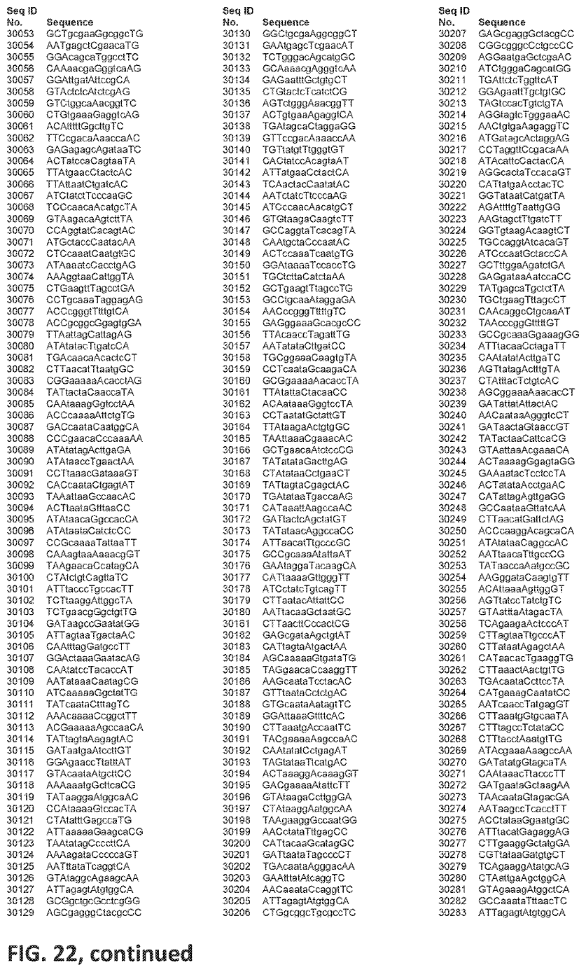

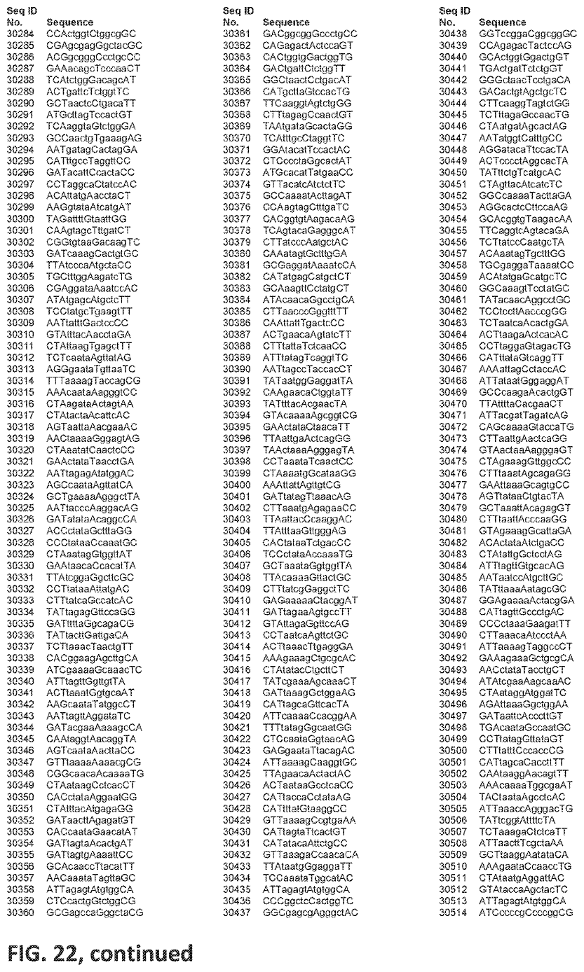

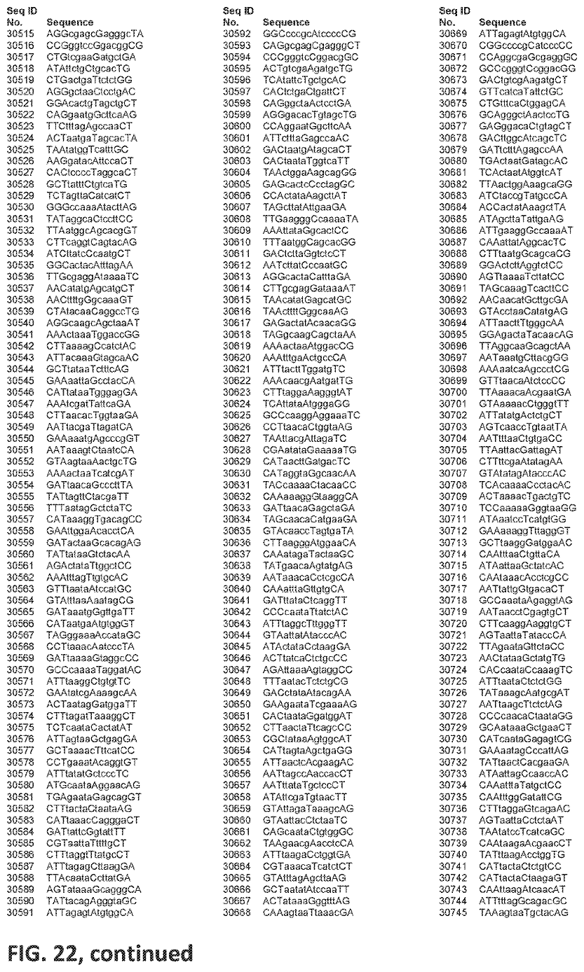

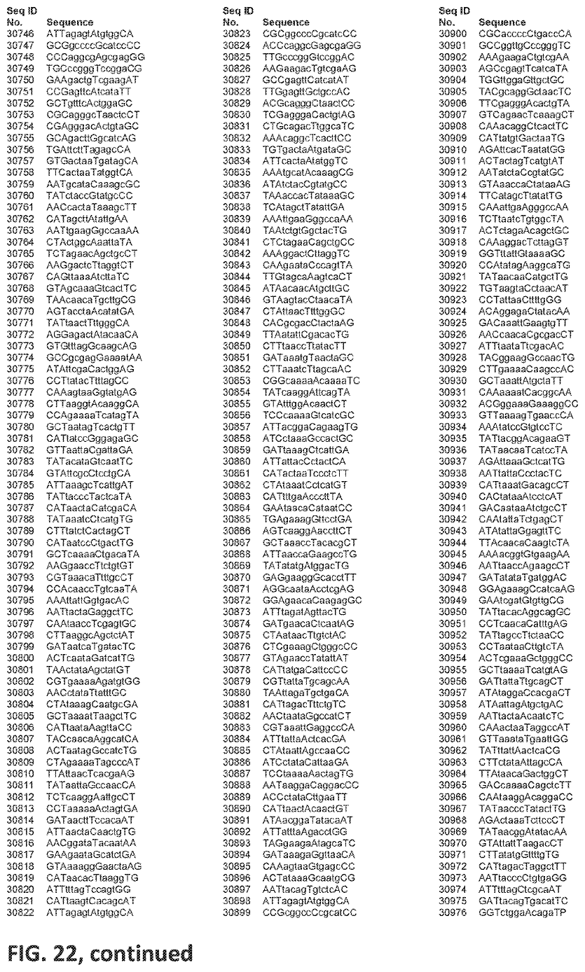

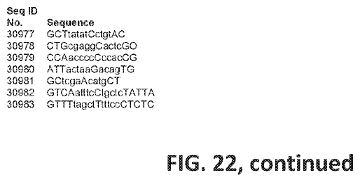

Exemplary modified multi-segmented antisense oligonucleotides of the disclosure include, but are not limited to, those exemplified in Table 2.1, Table 1A, Table 1B, Table 1C, FIG. 21 and FIG. 22.

Table 2.2 shows a legend for the modifications at each of the residues shown in Table 2.1, Table 1A and Table 1B. In some embodiments, the modified multi-segmented antisense oligonucleotide comprises the sequence of any one of SEQ ID NOS: 668-677, 679-682, 684-688, 690-692, 698-1107, 1109-1120, 1122-1125, 1127-1138, 1140, 1145-1156, 1158-1161, 1163-1174, 1176-1179, 1181-1192, 1194-1197, 1199-1210, 1212-1215, 1217-1228, 1230-1233, 1235-1246, 1248-1251, 1253-1264, 1266-1269, 1271-1282, 1284-1287, 1289-1300, 1302-1305, 1307-1318, 1320-1323, 1325-1336, 1338-1341, 1343-1354, 1356-1359, 1361-1372, 1374-1377, 1379-1390, 1392-1395, 1397-1408, 1410-1413, 1415-1426, and 1428-1431.

TABLE 2.1
Exemplary Modified multi-segmented antisense oligonucleotides
of the Disclosure

| SEQ | | | |
|---|---|---|---|
| ID | | | Target |
| NO | AUS# | Sequence | Gene |
| 667 | AUS-P1000 | GGGCTcatagcacatTATCC | PCSK9 |
| 668 | AUS-P1001 | GGGCtcataGcacatTATCC | PCSK9 |
| 669 | AUS-P1002 | GGGCTcataGcacatTATCC | PCSK9 |
| 670 | AUS-P1003 | GGGCtcataGcacatT+A+TCC | PCSK9 |
| 671 | AUS-P1004 | GGGCTcataGcacatT+A+TCC | PCSK9 |
| 672 | AUS-P1005 | GGGCTcatagCacatTATCC | PCSK9 |
| 673 | AUS-P1006 | GGGCtcataGcacatT+AT+CC | PCSK9 |
| 674 | AUS-P1007 | GGGCtcataGcacatT+ATC+C | PCSK9 |
| 675 | AUS-P1008 | GGGCTcatagCacatT+A+TCC | PCSK9 |
| 676 | AUS-P1009 | GGGCTcataGcacatT+ATC+C | PCSK9 |
| 677 | AUS-P1010 | GGGCTcatagCacatT+ATC+C | PCSK9 |
| 678 | AUS-D1000 | ACAGAcaataaatacCGAGG | DMPK |
| 679 | AUS-D1001 | ACAGacaatAaatacCGAGG | DMPK |
| 680 | AUS-D1002 | ACAGAcaatAaatacCGAGG | DMPK |
| 681 | AUS-D1003 | ACAGacaatAaatacC+G+AGG | DMPK |
| 682 | AUS-D1004 | ACAGAcaatAaatacC+G+AGG | DMPK |
| 683 | AUS-D1005 | ACAGacaataaatacCGAGG | DMPK |
| 684 | AUS-D1006 | ACAGacaataAatacCGAGG | DMPK |
| 685 | AUS-D1007 | ACAGAcaataAatacCGAGG | DMPK |
| 686 | AUS-D1008 | ACAGacaatAaatacC+GA+GG | DMPK |
| 687 | AUS-D1009 | ACAGacaatAaatacC+GAG+G | DMPK |
| 688 | AUS-D1010 | ACAGAcaataAatacC+G+AGG | DMPK |
| 689 | AUS-T1000 | TACAAatgggatgctACTGC | TTR |
| 690 | AUS-T1001 | TACAAatggGatgctACTGC | TTR |
| 691 | AUS-T1002 | TACAaatggGatgctACTGC | TTR |
| 692 | AUS-T1003 | TACAaatggGatgctA+C+TGC | TTR |
| 693 | AUS-T1004 | ACACAgtttgaatgaAATAG | TTR |
| 694 | AUS-T1005 | ACAGTttgaatgaaaTAGCA | TTR |
| 695 | AUS-T1006 | AGTTTgaatgaaataGCAGG | TTR |
| 696 | AUS-T1007 | TTTGAatgaaatagcAGGTG | TTR |
| 697 | AUS-A1000 | ACCCAattcagaaggAAGGA | AAT |
| 698 | AUS-A1001 | ACCCAattcAgaaggAAGGA | AAT |
| 699 | AUS-A1002 | ACCCaattcAgaaggAAGGA | AAT |
| 700 | AUS-A1003 | ACCCaattcAgaaggA+A+GGA | AAT |
| 1108 | AUS-PA1000 | CCTTGgccacgccggCATCC | PCSK9 |
| 1109 | AUS-PA1001 | CCTTggccaCgccggCATCC | PCSK9 |
| 1110 | AUS-PA1002 | CCTTggccaCgccggC+A+TCC | PCSK9 |
| 1111 | AUS-PA1003 | CCTTggccaCgccggC+AT+CC | PCSK9 |
| 1112 | AUS-PA1004 | CCTTggccaCgccggC+ATC+C | PCSK9 |
| 1113 | AUS-PA1005 | CCTTGgccaCgccggCATCC | PCSK9 |
| 1114 | AUS-PA1006 | CCTTGgccaCgccggC+A+TCC | PCSK9 |
| 1115 | AUS-PA1007 | CCTTGgccaCgccggC+AT+CC | PCSK9 |
| 1116 | AUS-PA1008 | CCTTGgccaCgccggC+ATC+C | PCSK9 |
| 1117 | AUS-PA1009 | CCTTGgccacGccggCATCC | PCSK9 |
| 1118 | AUS-PA10010 | CCTTGgccacGccggC+A+TCC | PCSK9 |
| 1119 | AUS-PA10011 | CCTTGgccacGccggC+AT+CC | PCSK9 |
| 1120 | AUS-PA10012 | CCTTGgccacGccggC+ATC+C | PCSK9 |
| 1121 | AUS-PA10013 | CCTTggccacgccggCATCC | PCSK9 |
| 1122 | AUS-PA10014 | CCTTggccacGccggCATCC | PCSK9 |
| 1123 | AUS-PA10015 | CCTTggccacGccggC+A+TCC | PCSK9 |
| 1124 | AUS-PA10016 | CCTTggccacGccggC+AT+CC | PCSK9 |
| 1125 | AUS-PA10017 | CCTTggccacGccggC+ATC+C | PCSK9 |
| 1126 | AUS-DA1000 | ACAGAcaataaatacCGAGG | DMPK |
| 1127 | AUS-DA1001 | ACAGacaatAaatacCGAGG | DMPK |
| 1128 | AUS-DA1002 | ACAGacaatAaatacC+G+AGG | DMPK |
| 1129 | AUS-DA1003 | ACAGacaatAaatacC+GA+GG | DMPK |
| 1130 | AUS-DA1004 | ACAGacaatAaatacC+GAG+G | DMPK |
| 1131 | AUS-DA1005 | ACAGAcaatAaatacCGAGG | DMPK |
| 1132 | AUS-DA1006 | ACAGAcaatAaatacC+G+AGG | DMPK |
| 1133 | AUS-DA1007 | ACAGAcaatAaatacC+GA+GG | DMPK |
| 1134 | AUS-DA1008 | ACAGAcaatAaatacC+GAG+G | DMPK |
| 1135 | AUS-DA1009 | ACAGAcaataAatacCGAGG | DMPK |
| 1136 | AUS-DA10010 | ACAGAcaataAatacC+G+AGG | DMPK |
| 1137 | AUS-DA10011 | ACAGAcaataAatacC+GA+GG | DMPK |
| 1138 | AUS-DA10012 | ACAGAcaataAatacC+GAG+G | DMPK |
| 1139 | AUS-DA10013 | ACAGacaataaatacCGAGG | DMPK |
| 1140 | AUS-DA10014 | ACAGacaataAatacCGAGG | DMPK |
| 1141 | AUS-DA10015 | ACAGAcaataaatacC+G+AGG | DMPK |
| 1142 | AUS-DA10016 | ACAGAcaataaatacC+GA+GG | DMPK |
| 1143 | AUS-DA10017 | ACAGAcaataaatacC+GAG+G | DMPK |
| 1144 | AUS-DB1000 | CGGAGcggttgtgaaCTGGC | DMPK |
| 1145 | AUS-DB1001 | CGGAgcggtTgtgaaCTGGC | DMPK |
| 1146 | AUS-DB1002 | CGGAgcggtTgtgaaC+T+GGC | DMPK |
| 1147 | AUS-DB1003 | CGGAgcggtTgtgaaC+TG+GC | DMPK |
| 1148 | AUS-DB1004 | CGGAgcggtTgtgaaC+TGG+C | DMPK |
| 1149 | AUS-DB1005 | CGGAGcggtTgtgaaCTGGC | DMPK |
| 1150 | AUS-DB1006 | CGGAGcggtTgtgaaC+T+GGC | DMPK |
| 1151 | AUS-DB1007 | CGGAGcggtTgtgaaC+TG+GC | DMPK |
| 1152 | AUS-DB1008 | CGGAGcggtTgtgaaC+TGG+C | DMPK |
| 1153 | AUS-DB1009 | CGGAGcggttGtgaaCTGGC | DMPK |
| 1154 | AUS-DB10010 | CGGAGcggttGtgaaC+T+GGC | DMPK |
| 1155 | AUS-DB10011 | CGGAGcggttGtgaaC+TG+GC | DMPK |
| 1156 | AUS-DB10012 | CGGAGcggttGtgaaC+TGG+C | DMPK |
| 1157 | AUS-DB10013 | CGGAgcggttgtgaaCTGGC | DMPK |
| 1158 | AUS-DB10014 | CGGAgcggttGtgaaCTGGC | DMPK |
| 1159 | AUS-DB10015 | CGGAgcggttGtgaaC+T+GGC | DMPK |
| 1160 | AUS-DB10016 | CGGAgcggttGtgaaC+TG+GC | DMPK |
| 1161 | AUS-DB10017 | CGGAgcggttGtgaaC+TGG+C | DMPK |
| 1162 | AUS-DC1000 | GCGUAgaagggcgucUGCCC | DMPK |
| 1163 | AUS-DC1001 | GCGUagaagGgcgucUGCCC | DMPK |
| 1164 | AUS-DC1002 | GCGUagaagGgcgucU+G+CCC | DMPK |
| 1165 | AUS-DC1003 | GCGUagaagGgcgucU+GC+CC | DMPK |
| 1166 | AUS-DC1004 | GCGUagaagGgcgucU+GCC+C | DMPK |
| 1167 | AUS-DC1005 | GCGUAgaagGgcgucUGCCC | DMPK |
| 1168 | AUS-DC1006 | GCGUAgaagGgcgucU+G+CCC | DMPK |
| 1169 | AUS-DC1007 | GCGUAgaagGgcgucU+GC+CC | DMPK |
| 1170 | AUS-DC1008 | GCGUAgaagGgcgucU+GCC+C | DMPK |
| 1171 | AUS-DC1009 | GCGUAgaaggGcgucUGCCC | DMPK |
| 1172 | AUS-DC10010 | GCGUAgaaggGcgucU+G+CCC | DMPK |
| 1173 | AUS-DC10011 | GCGUAgaaggGcgucU+GC+CC | DMPK |
| 1174 | AUS-DC10012 | GCGUAgaaggGcgucU+GCC+C | DMPK |
| 1175 | AUS-DC10013 | GCGUagaagggcgucUGCCC | DMPK |
| 1176 | AUS-DC10014 | GCGUagaaggGcgucUGCCC | DMPK |
| 1177 | AUS-DC10015 | GCGUagaaggGcgucU+G+CCC | DMPK |
| 1178 | AUS-DC10016 | GCGUagaaggGcgucU+GC+CC | DMPK |
| 1179 | AUS-DC10017 | GCGUagaaggGcgucU+GCC+C | DMPK |
| 1180 | AUS-TA1000 | UCUUGgttacatgaaAUCCC | TTR |
| 1181 | AUS-TA1001 | UCUUggttaCatgaaAUCCC | TTR |
| 1182 | AUS-TA1002 | UCUUggttaCatgaaA+U+CCC | TTR |
| 1183 | AUS-TA1003 | UCUUggttaCatgaaA+UC+CC | TTR |
| 1184 | AUS-TA1004 | UCUUggttaCatgaaA+UCC+C | TTR |
| 1185 | AUS-TA1005 | UCUUGgttaCatgaaAUCCC | TTR |
| 1186 | AUS-TA1006 | UCUUGgttaCatgaaA+U+CCC | TTR |
| 1187 | AUS-TA1007 | UCUUGgttaCatgaaA+UC+CC | TTR |
| 1188 | AUS-TA1008 | UCUUGgttaCatgaaA+UCC+C | TTR |
| 1189 | AUS-TA1009 | UCUUGgttacAtgaaAUCCC | TTR |
| 1190 | AUS-TA10010 | UCUUGgttacAtgaaA+U+CCC | TTR |
| 1191 | AUS-TA10011 | UCUUGgttacAtgaaA+UC+CC | TTR |
| 1192 | AUS-TA10012 | UCUUGgttacAtgaaA+UCC+C | TTR |
| 1193 | AUS-TA10013 | UCUUggttacatgaaAUCCC | TTR |
| 1194 | AUS-TA10014 | UCUUggttacAtgaaAUCCC | TTR |
| 1195 | AUS-TA10015 | UCUUggttacAtgaaA+U+CCC | TTR |
| 1196 | AUS-TA10016 | UCUUggttacAtgaaA+UC+CC | TTR |
| 1197 | AUS-TA10017 | UCUUggttacAtgaaA+UCC+C | TTR |
| 1198 | AUS-AA1000 | TGTTAaacatgcctaaACGCT | AAT |
| 1199 | AUS-AA1001 | TGTTaaacaTgcctaaACGCT | AAT |
| 1200 | AUS-AA1002 | TGTTaaacaTgcctaaA+C+GCT | AAT |
| 1201 | AUS-AA1003 | TGTTaaacaTgcctaaA+CG+CT | AAT |
| 1202 | AUS-AA1004 | TGTTaaacaTgcctaaA+CGC+T | AAT |
| 1203 | AUS-AA1005 | TGTTAaacaTgcctaaACGCT | AAT |
| 1204 | AUS-AA1006 | TGTTAaacaTgcctaaA+C+GCT | AAT |
| 1205 | AUS-AA1007 | TGTTAaacaTgcctaaA+CG+CT | AAT |
| 1206 | AUS-AA1008 | TGTTAaacaTgcctaaA+CGC+T | AAT |
| 1207 | AUS-AA1009 | TGTTAaacatGcctaaACGCT | AAT |
| 1208 | AUS-AA10010 | TGTTAaacatGcctaaA+C+GCT | AAT |
| 1209 | AUS-AA10011 | TGTTAaacatGcctaaA+CG+CT | AAT |
| 1210 | AUS-AA10012 | TGTTAaacatGcctaaA+CGC+T | AAT |
| 1211 | AUS-AA10013 | TGTTaaacatgcctaaACGCT | AAT |
| 1212 | AUS-AA10014 | TGTTaaacatGcctaaACGCT | AAT |
| 1213 | AUS-AA10015 | TGTTaaacatGcctaaA+C+GCT | AAT |
| 1214 | AUS-AA10016 | TGTTaaacatGcctaaA+CG+CT | AAT |
| 1215 | AUS-AA10017 | TGTTaaacatGcctaaA+CGC+T | AAT |
| 1216 | AUS-AB1000 | GCCTCagtctgcttcGCACC | ApoB |
| 1217 | AUS-AB1001 | GCCTcagtcTgcttcGCACC | ApoB |
| 1218 | AUS-AB1002 | GCCTcagtcTgcttcG+C+ACC | ApoB |
| 1219 | AUS-AB1003 | GCCTcagtcTgcttcG+CA+CC | ApoB |
| 1220 | AUS-AB1004 | GCCTcagtcTgcttcG+CAC+C | ApoB |
| 1221 | AUS-AB1005 | GCCTCagtcTgcttcGCACC | ApoB |
| 1222 | AUS-AB1006 | GCCTCagtcTgcttcG+C+ACC | ApoB |
| 1223 | AUS-AB1007 | GCCTCagtcTgcttcG+CA+CC | ApoB |
| 1224 | AUS-AB1008 | GCCTCagtcTgcttcG+CAC+C | ApoB |
| 1225 | AUS-AB1009 | GCCTCagtctGcttcGCACC | ApoB |
| 1226 | AUS-AB10010 | GCCTCagtctGcttcG+C+ACC | ApoB |
| 1227 | AUS-AB10011 | GCCTCagtctGcttcG+CA+CC | ApoB |
| 1228 | AUS-AB10012 | GCCTCagtctGcttcG+CAC+C | ApoB |
| 1229 | AUS-AB10013 | GCCTcagtctgcttcGCACC | ApoB |
| 1230 | AUS-AB10014 | GCCTcagtctGcttcGCACC | ApoB |
| 1231 | AUS-AB10015 | GCCTcagtctGcttcG+C+ACC | ApoB |
| 1232 | AUS-AB10016 | GCCTcagtctGcttcG+CA+CC | ApoB |
| 1233 | AUS-AB10017 | GCCTcagtctGcttcG+CAC+C | ApoB |
| 1234 | AUS-AC1000 | AGCTTcttgtccagcTTTAT | ApoCIII |
| 1235 | AUS-AC1001 | AGCTtcttgTccagcTTTAT | ApoCIII |
| 1236 | AUS-AC1002 | AGCTtcttgTccagcT+T+TAT | ApoCIII |
| 1237 | AUS-AC1003 | AGCTtcttgTccagcT+TT+AT | ApoCIII |
| 1238 | AUS-AC1004 | AGCTtcttgTccagcT+TTA+T | ApoCIII |
| 1239 | AUS-AC1005 | AGCTTcttgTccagcTTTAT | ApoCIII |
| 1240 | AUS-AC1006 | AGCTTcttgTccagcT+T+TAT | ApoCIII |
| 1241 | AUS-AC1007 | AGCTTcttgTccagcT+TT+AT | ApoCIII |
| 1242 | AUS-AC1008 | AGCTTcttgTccagcT+TTA+T | ApoCIII |
| 1243 | AUS-AC1009 | AGCTTcttgtCcagcTTTAT | ApoCIII |
| 1244 | AUS-AC10010 | AGCTTcttgtCcagcT+T+TAT | ApoCIII |
| 1245 | AUS-AC10011 | AGCTTcttgtCcagcT+TT+AT | ApoCIII |
| 1246 | AUS-AC10012 | AGCTTcttgtCcagcT+TTA+T | ApoCIII |
| 1247 | AUS-AC10013 | AGCTtcttgtccagcTTTAT | ApoCIII |
| 1248 | AUS-AC10014 | AGCTtcttgtCcagcTTTAT | ApoCIII |
| 1249 | AUS-AC10015 | AGCTtcttgtCcagcT+T+TAT | ApoCIII |
| 1250 | AUS-AC10016 | AGCTtcttgtCcagcT+TT+AT | ApoCIII |
| 1251 | AUS-AC10017 | AGCTtcttgtCcagcT+TTA+T | ApoCIII |
| 1252 | AUS-TB1000 | GCTGAttagagagagGTCCC | TNF-alpha |
| 1253 | AUS-TB1001 | GCTGattagAgagagGTCCC | TNF-alpha |
| 1254 | AUS-TB1002 | GCTGattagAgagagG+T+CCC | TNF-alpha |
| 1255 | AUS-TB1003 | GCTGattagAgagagG+TC+CC | TNF-alpha |
| 1256 | AUS-TB1004 | GCTGattagAgagagG+TCC+C | TNF-alpha |
| 1257 | AUS-TB1005 | GCTGAttagAgagagGTCCC | TNF-alpha |
| 1258 | AUS-TB1006 | GCTGAttagAgagagG+T+CCC | TNF-alpha |
| 1259 | AUS-TB1007 | GCTGAttagAgagagG+TC+CC | TNF-alpha |
| 1260 | AUS-TB1008 | GCTGAttagAgagagG+TCC+C | TNF-alpha |
| 1261 | AUS-TB1009 | GCTGAttagaGagagGTCCC | TNF-alpha |
| 1262 | AUS-TB10010 | GCTGAttagaGagagG+T+CCC | TNF-alpha |
| 1263 | AUS-TB10011 | GCTGAttagaGagagG+TC+CC | TNF-alpha |
| 1264 | AUS-TB10012 | GCTGAttagaGagagG+TCC+C | TNF-alpha |
| 1265 | AUS-TB10013 | GCTGattagagagagGTCCC | TNF-alpha |
| 1266 | AUS-TB10014 | GCTGattagaGagagGTCCC | TNF-alpha |
| 1267 | AUS-TB10015 | GCTGattagaGagagG+T+CCC | TNF-alpha |
| 1268 | AUS-TB10016 | GCTGattagaGagagG+TC+CC | TNF-alpha |
| 1269 | AUS-TB10017 | GCTGattagaGagagG+TCC+C | TNF-alpha |
| 1270 | AUS-TC1000 | AAGAGgacctgggagTAGAT | TNF-alpha |
| 1271 | AUS-TC1001 | AAGAggaccTgggagTAGAT | TNF-alpha |
| 1272 | AUS-TC1002 | AAGAggaccTgggagT+A+GAT | TNF-alpha |
| 1273 | AUS-TC1003 | AAGAggaccTgggagT+AG+AT | TNF-alpha |
| 1274 | AUS-TC1004 | AAGAggaccTgggagT+AGA+T | TNF-alpha |
| 1275 | AUS-TC1005 | AAGAGgaccTgggagTAGAT | TNF-alpha |
| 1276 | AUS-TC1006 | AAGAGgaccTgggagT+A+GAT | TNF-alpha |
| 1277 | AUS-TC1007 | AAGAGgaccTgggagT+AG+AT | TNF-alpha |
| 1278 | AUS-TC1008 | AAGAGgaccTgggagT+AGA+T | TNF-alpha |
| 1279 | AUS-TC1009 | AAGAGgacctGggagTAGAT | TNF-alpha |
| 1280 | AUS-TC10010 | AAGAGgacctGggagT+A+GAT | TNF-alpha |
| 1281 | AUS-TC10011 | AAGAGgacctGggagT+AG+AT | TNF-alpha |
| 1282 | AUS-TC10012 | AAGAGgacctGggagT+AGA+T | TNF-alpha |
| 1283 | AUS-TC10013 | AAGAggacctgggagTAGAT | TNF-alpha |
| 1284 | AUS-TC10014 | AAGAggacctGggagTAGAT | TNF-alpha |
| 1285 | AUS-TC10015 | AAGAggacctGggagT+A+GAT | TNF-alpha |
| 1286 | AUS-TC10016 | AAGAggacctGggagT+AG+AT | TNF-alpha |
| 1287 | AUS-TC10017 | AAGAggacctGggagT+AGA+T | TNF-alpha |
| 1288 | AUS-AG1000 | CACAAacaagctggtCGGUU | AGT |
| 1289 | AUS-AG1001 | CACAaacaaGctggtCGGUU | AGT |
| 1290 | AUS-AG1002 | CACAaacaaGctggtC+G+GUU | AGT |
| 1291 | AUS-AG1003 | CACAaacaaGctggtC+GG+UU | AGT |
| 1292 | AUS-AG1004 | CACAaacaaGctggtC+GGU+U | AGT |
| 1293 | AUS-AG1005 | CACAAacaaGctggtCGGUU | AGT |
| 1294 | AUS-AG1006 | CACAAacaaGctggtC+G+GUU | AGT |
| 1295 | AUS-AG1007 | CACAAacaaGctggtC+GG+UU | AGT |
| 1296 | AUS-AG1008 | CACAAacaaGctggtC+GGU+U | AGT |
| 1297 | AUS-AG1009 | CACAAacaagCtggtCGGUU | AGT |
| 1298 | AUS-AG10010 | CACAAacaagCtggtC+G+GUU | AGT |
| 1299 | AUS-AG10011 | CACAAacaagCtggtC+GG+UU | AGT |
| 1300 | AUS-AG10012 | CACAAacaagCtggtC+GGU+U | AGT |
| 1301 | AUS-AG10013 | CACAaacaagctggtCGGUU | AGT |
| 1302 | AUS-AG10014 | CACAaacaagCtggtCGGUU | AGT |
| 1303 | AUS-AG10015 | CACAaacaagCtggtC+G+GUU | AGT |
| 1304 | AUS-AG10016 | CACAaacaagCtggtC+GG+UU | AGT |
| 1305 | AUS-AG10017 | CACAaacaagCtggtC+GGU+U | AGT |
| 1306 | AUS-CF1000 | ATCCCacgcccctgtCCAGC | CFB |
| 1307 | AUS-CF1001 | ATCCcacgcCcctgtCCAGC | CFB |
| 1308 | AUS-CF1002 | ATCCcacgcCcctgtC+C+AGC | CFB |
| 1309 | AUS-CF1003 | ATCCcacgcCcctgtC+CA+GC | CFB |
| 1310 | AUS-CF1004 | ATCCcacgcCcctgtC+CAG+C | CFB |
| 1311 | AUS-CF1005 | ATCCCacgcCcctgtCCAGC | CFB |
| 1312 | AUS-CF1006 | ATCCCacgcCcctgtC+C+AGC | CFB |
| 1313 | AUS-CF1007 | ATCCCacgcCcctgtC+CA+GC | CFB |
| 1314 | AUS-CF1008 | ATCCCacgcCcctgtC+CAG+C | CFB |
| 1315 | AUS-CF1009 | ATCCCacgccCctgtCCAGC | CFB |
| 1316 | AUS-CF10010 | ATCCCacgccCctgtC+C+AGC | CFB |
| 1317 | AUS-CF10011 | ATCCCacgccCctgtC+CA+GC | CFB |
| 1318 | AUS-CF10012 | ATCCCacgccCctgtC+CAG+C | CFB |
| 1319 | AUS-CF10013 | ATCCcacgcccctgtCCAGC | CFB |
| 1320 | AUS-CF10014 | ATCCcacgccCctgtCCAGC | CFB |
| 1321 | AUS-CF10015 | ATCCcacgccCctgtC+C+AGC | CFB |
| 1322 | AUS-CF10016 | ATCCcacgccCctgtC+CA+GC | CFB |
| 1323 | AUS-CF10017 | ATCCcacgccCctgtC+CAG+C | CFB |
| 1324 | AUS-DG1000 | TGCCAtttaatgagcTTCAC | DGAT2 |
| 1325 | AUS-DG1001 | TGCCatttaAtgagcTTCAC | DGAT2 |
| 1326 | AUS-DG1002 | TGCCatttaAtgagcT+T+CAC | DGAT2 |
| 1327 | AUS-DG1003 | TGCCatttaAtgagcT+TC+AC | DGAT2 |
| 1328 | AUS-DG1004 | TGCCatttaAtgagcT+TCA+C | DGAT2 |
| 1329 | AUS-DG1005 | TGCCAtttaAtgagcTTCAC | DGAT2 |
| 1330 | AUS-DG1006 | TGCCAtttaAtgagcT+T+CAC | DGAT2 |
| 1331 | AUS-DG1007 | TGCCAtttaAtgagcT+TC+AC | DGAT2 |
| 1332 | AUS-DG1008 | TGCCAtttaAtgagcT+TCA+C | DGAT2 |
| 1333 | AUS-DG1009 | TGCCAtttaaTgagcTTCAC | DGAT2 |
| 1334 | AUS-DG10010 | TGCCAtttaaTgagcT+T+CAC | DGAT2 |
| 1335 | AUS-DG10011 | TGCCAtttaaTgagcT+TC+AC | DGAT2 |
| 1336 | AUS-DG10012 | TGCCAtttaaTgagcT+TCA+C | DGAT2 |
| 1337 | AUS-DG10013 | TGCCatttaatgagcTTCAC | DGAT2 |
| 1338 | AUS-DG10014 | TGCCatttaaTgagcTTCAC | DGAT2 |
| 1339 | AUS-DG10015 | TGCCatttaaTgagcT+T+CAC | DGAT2 |
| 1340 | AUS-DG10016 | TGCCatttaaTgagcT+TC+AC | DGAT2 |
| 1341 | AUS-DG10017 | TGCCatttaaTgagcT+TCA+C | DGAT2 |
| 1342 | AUS-PN1000 | TACTTtattcaatgtGGCTT | PNPLA3 |
| 1343 | AUS-PN1001 | TACTttattCaatgtGGCTT | PNPLA3 |
| 1344 | AUS-PN1002 | TACTttattCaatgtG+G+CTT | PNPLA3 |
| 1345 | AUS-PN1003 | TACTttattCaatgtG+GC+TT | PNPLA3 |
| 1346 | AUS-PN1004 | TACTttattCaatgtG+GCT+T | PNPLA3 |
| 1347 | AUS-PN1005 | TACTTtattCaatgtGGCTT | PNPLA3 |
| 1348 | AUS-PN1006 | TACTTtattCaatgtG+G+CTT | PNPLA3 |
| 1349 | AUS-PN1007 | TACTTtattCaatgtG+GC+TT | PNPLA3 |
| 1350 | AUS-PN1008 | TACTTtattCaatgtG+GCT+T | PNPLA3 |
| 1351 | AUS-PN1009 | TACTTtattcAatgtGGCTT | PNPLA3 |
| 1352 | AUS-PN10010 | TACTTtattcAatgtG+G+CTT | PNPLA3 |
| 1353 | AUS-PN10011 | TACTTtattcAatgtG+GC+TT | PNPLA3 |
| 1354 | AUS-PN10012 | TACTTtattcAatgtG+GCT+T | PNPLA3 |
| 1355 | AUS-PN10013 | TACTttattcaatgtGGCTT | PNPLA3 |
| 1356 | AUS-PN10014 | TACTttattcAatgtGGCTT | PNPLA3 |
| 1357 | AUS-PN10015 | TACTttattcAatgtG+G+CTT | PNPLA3 |
| 1358 | AUS-PN10016 | TACTttattcAatgtG+GC+TT | PNPLA3 |
| 1359 | AUS-PN10017 | TACTttattcAatgtG+GCT+T | PNPLA3 |
| 1360 | AUS-PB1000 | ACAAAagcaaaacagGUCUA | PCSK9 |
| 1361 | AUS-PB1001 | ACAAaagcaAaacagGUCUA | PCSK9 |
| 1362 | AUS-PB1002 | ACAAaagcaAaacagG+U+CUA | PCSK9 |
| 1363 | AUS-PB1003 | ACAAaagcaAaacagG+UC+UA | PCSK9 |
| 1364 | AUS-PB1004 | ACAAaagcaAaacagG+UCU+A | PCSK9 |
| 1365 | AUS-PB1005 | ACAAAagcaAaacagGUCUA | PCSK9 |
| 1366 | AUS-PB1006 | ACAAAagcaAaacagG+U+CUA | PCSK9 |
| 1367 | AUS-PB1007 | ACAAAagcaAaacagG+UC+UA | PCSK9 |
| 1368 | AUS-PB1008 | ACAAAagcaAaacagG+UCU+A | PCSK9 |
| 1369 | AUS-PB1009 | ACAAAagcaaAacagGUCUA | PCSK9 |
| 1370 | AUS-PB10010 | ACAAAagcaaAacagG+U+CUA | PCSK9 |
| 1371 | AUS-PB10011 | ACAAAagcaaAacagG+UC+UA | PCSK9 |
| 1372 | AUS-PB10012 | ACAAAagcaaAacagG+UCU+A | PCSK9 |
| 1373 | AUS-PB10013 | ACAAaagcaaaacagGUCUA | PCSK9 |
| 1374 | AUS-PB10014 | ACAAaagcaaAacagGUCUA | PCSK9 |
| 1375 | AUS-PB10015 | ACAAaagcaaAacagG+U+CUA | PCSK9 |
| 1376 | AUS-PB10016 | ACAAaagcaaAacagG+UC+UA | PCSK9 |
| 1377 | AUS-PB10017 | ACAAaagcaaAacagG+UCU+A | PCSK9 |
| 1378 | AUS-AL1000 | UAAGAugagacacucUUUCU | ALAS1 |
| 1379 | AUS-AL1001 | UAAGaugagAcacucUUUCU | ALAS1 |
| 1380 | AUS-AL1002 | UAAGaugagAcacucU+U+UCU | ALAS1 |
| 1381 | AUS-AL1003 | UAAGaugagAcacucU+UU+CU | ALAS1 |
| 1382 | AUS-AL1004 | UAAGaugagAcacucU+UUC+U | ALAS1 |
| 1383 | AUS-AL1005 | UAAGAugagAcacucUUUCU | ALAS1 |
| 1384 | AUS-AL1006 | UAAGAugagAcacucU+U+UCU | ALAS1 |
| 1385 | AUS-AL1007 | UAAGAugagAcacucU+UU+CU | ALAS1 |
| 1386 | AUS-AL1008 | UAAGAugagAcacucU+UUC+U | ALAS1 |
| 1387 | AUS-AL1009 | UAAGAugagaCacucUUUCU | ALAS1 |
| 1388 | AUS-AL10010 | UAAGAugagaCacucU+U+UCU | ALAS1 |
| 1389 | AUS-AL10011 | UAAGAugagaCacucU+UU+CU | ALAS1 |
| 1390 | AUS-AL10012 | UAAGAugagaCacucU+UUC+U | ALAS1 |
| 1391 | AUS-AL10013 | UAAGaugagacacucUUUCU | ALAS1 |
| 1392 | AUS-AL10014 | UAAGaugagaCacucUUUCU | ALAS1 |
| 1393 | AUS-AL10015 | UAAGaugagaCacucU+U+UCU | ALAS1 |
| 1394 | AUS-AL10016 | UAAGaugagaCacucU+UU+CU | ALAS1 |
| 1395 | AUS-AL10017 | UAAGaugagaCacucU+UUC+U | ALAS1 |
| 1396 | AUS-TD1000 | AUGGAauacucuuggUUACT | TTR |
| 1397 | AUS-TD1001 | AUGGaauacUcuuggUUACT | TTR |
| 1398 | AUS-TD1002 | AUGGaauacUcuuggU+U+ACT | TTR |
| 1399 | AUS-TD1003 | AUGGaauacUcuuggU+UA+CT | TTR |
| 1400 | AUS-TD1004 | AUGGaauacUcuuggU+UAC+T | TTR |
| 1401 | AUS-TD1005 | AUGGAauacUcuuggUUACT | TTR |
| 1402 | AUS-TD1006 | AUGGAauacUcuuggU+U+ACT | TTR |
| 1403 | AUS-TD1007 | AUGGAauacUcuuggU+UA+CT | TTR |
| 1404 | AUS-TD1008 | AUGGAauacUcuuggU+UAC+T | TTR |
| 1405 | AUS-TD1009 | AUGGAauacuCuuggUUACT | TTR |
| 1406 | AUS-TD10010 | AUGGAauacuCuuggU+U+ACT | TTR |
| 1407 | AUS-TD10011 | AUGGAauacuCuuggU+UA+CT | TTR |
| 1408 | AUS-TD10012 | AUGGAauacuCuuggU+UAC+T | TTR |
| 1409 | AUS-TD10013 | AUGGaauacucuuggUUACT | TTR |
| 1410 | AUS-TD10014 | AUGGaauacuCuuggUUACT | TTR |
| 1411 | AUS-TD10015 | AUGGaauacuCuuggU+U+ACT | TTR |
| 1412 | AUS-TD10016 | AUGGaauacuCuuggU+UA+CT | TTR |
| 1413 | AUS-TD10017 | AUGGaauacuCuuggU+UAC+T | TTR |
| 1414 | AUS-H1000 | UAUAUuuccaggaugAAAGU | hydroxy acid |
| | | | oxidase 1 |
| 1415 | AUS-H1001 | UAUAuuuccAggaugAAAGU | hydroxy acid |
| | | | oxidase 1 |
| 1416 | AUS-H1002 | UAUAuuuccAggaugA+A+AGU | hydroxy acid |
| | | | oxidase 1 |
| 1417 | AUS-H1003 | UAUAuuuccAggaugA+AA+GU | hydroxy acid |
| | | | oxidase 1 |
| 1418 | AUS-H1004 | UAUAuuuccAggaugA+AAG+U | hydroxy acid |
| | | | oxidase 1 |
| 1419 | AUS-H1005 | UAUAUuuccAggaugAAAGU | hydroxy acid |
| | | | oxidase 1 |
| 1420 | AUS-H1006 | UAUAUuuccAggaugA+A+AGU | hydroxy acid |
| | | | oxidase 1 |
| 1421 | AUS-H1007 | UAUAUuuccAggaugA+AA+GU | hydroxy acid |
| | | | oxidase 1 |
| 1422 | AUS-H1008 | UAUAUuuccAggaugA+AAG+U | hydroxy acid |
| | | | oxidase 1 |
| 1423 | AUS-H1009 | UAUAUuuccaGgaugAAAGU | hydroxy acid |
| | | | oxidase 1 |
| 1424 | AUS-H10010 | UAUAUuuccaGgaugA+A+AGU | hydroxy acid |
| | | | oxidase 1 |
| 1425 | AUS-H10011 | UAUAUuuccaGgaugA+AA+GU | hydroxy acid |
| | | | oxidase 1 |
| 1426 | AUS-H10012 | UAUAUuuccaGgaugA+AAG+U | hydroxy acid |
| | | | oxidase 1 |
| 1427 | AUS-H10013 | UAUAuuuccaggaugAAAGU | hydroxy acid |
| | | | oxidase 1 |
| 1428 | AUS-H10014 | UAUAuuuccaGgaugAAAGU | hydroxy acid |
| | | | oxidase 1 |
| 1429 | AUS-H10015 | UAUAuuuccaGgaugA+A+AGU | hydroxy acid |
| | | | oxidase 1 |
| 1430 | AUS-H10016 | UAUAuuuccaGgaugA+AA+GU | hydroxy acid |
| | | | oxidase 1 |
| 1431 | AUS-H10017 | UAUAuuuccaGgaugA+AAG+U | hydroxy acid |
| | | | oxidase 1 |

TABLE 2.2
Legend of Modifications
Legend

| Symbol | Description | Representation | Examples |
|---|---|---|---|
| n | lower case | 2′-deoxy nucleoside | c stands for 2′-deoxy 5-methylcytidine. |
| | | | a stands for 2′-deoxy adenosine. |
| | | | g stands for 2′-deoxy guanosine, and |
| | | | u stands for 2′-deoxy thymidine. |
| N | UPPER CASE | 2′-O-methoxyethyl | C stands for 2′-O-methoxyethyl 5-methylcytidine. |
| | | nucleoside | A stands for 2′-O-methoxyethyl adenosine. |
| | | | G stands for 2′-O-methoxyethyl guanosine. |
| | | | T stands for 2′-O-methoxyethyl thymidine, and |
| | | | U stands for 2′-O-methoxyethyl uridine |
| C | UPPER CASE, Underlined | 2′-O-methoxyethyl cytidine | C stands for 2′-O-methoxyethyl cytidine |
| | | | (namely regular unmethylated cytidine with 2′-O- |
| | | | methoxyethyl modification). |
| c | lower case c, underlined | 2′-deoxy cytidine | c stands for 2′-deoxy cytidine (namely regular |
| | | | unmethylated 2′-deoxy cytidine). |
| N | UPPER CASE ITALICIZED | 2′-O-methyl nucleoside | C stands for 2′-O-methyl 5-methylcytidine. |
| | | | A stands for 2′-O-methyl adenosine, |
| | | | G stands for 2′-O-methyl guanosine. |
| | | | T stands for 2′-O-methyl thymidine, and |
| | | | U stands for 2′-O-methyl uridine |
| n | lower case bold italicized | 2′-OH nucleoside | c stands for 2′-OH-5-methylcytidine |
| | | | (or 5-methy cytidine). |
| | | | a stands for 2′-OH adenosine (or adenosine). |
| | | | g stands for 2′-OH guanosine (or guanosine), and |
| | | | u stands for 2′-OH uridine for uridine). |
| N | UPPER CASE bold | Glycol nucleic acid (GNA) | A stands for a GNA with adenine base. |
| | | | C stands for a GINA with 5-methylcytosine base. |
| | | | +A stands for a LNA with adenine base. |
| | | | +C stands for a LNA with 5-methylcytosine base. |
| | | | +G stands for a LNA with guanine base. |
| +N | UPPER CASE with | Locked nucleic acid (LNA) | +T stands for a LNA with thymine base. |
| | a + sign in front | | +U stands for a LNA with uracil base |
| +C | UPPER CASE underlined | Locked nucleic acid (LNA) | +C stands for a LNA with regular |
| | with a + sign in [symbol] | with cytosine [symbol] | cytosine base (unmethylated) |
| | | | A′ stands for 2′-fluoro 2′-deoxy adenosine. |
| | | | C′′ stands for 2′-fluoro 2′-deoxy 5-methylcytidine. |
| N′ | UPPER CASE followed | 2′-Fluoro 2′-deoxy nucleoside | G′ stands for 2′-fluoro 2′-deoxy guanosine. |
| | by a ′ sign | | U′ stands for 2′-fluoro 2′-deoxy uridine |
| C′ | UPPER CASE underlined | 2′-Fluoro 2′-deoxy cytidine | C′ stands for 2′-fluoro 2′-deoxy |
| | followed by a ′ sign | | cytidine (unmethylated) |
| N′′ | UPPER CASE followed | 2′-F-Arabinonucleic | A′′ stands for a 2′-F-ANA with adenine base |
| | by a ′′ sign | Acid (2′-F-ANA) | T′′ stands for a 2′-F-ANA with thymine base |
| | | | G′′ stands for a 2′-F-ANA with guanine base |
| C′′ | UPPER CASE underlined | 2′-F-ANA with cytosine base | C′′ stands for a 2′-F-ANA with cytosine base |
| | followed by a ″ [symbol] | | |

[symbol]

indicates data missing or illegible when filed

### Conjugated Oligonucleotides

The oligonucleotides of the disclosure may be further covalently linked to one or more conjugate groups/moieties. In some embodiments, such groups enhance the activity, cellular distribution and/or cellular uptake of the resulting vaccines. Exemplary conjugate groups include, but are not limited to, carbohydrate moieties (e.g, N-Acetylgalactosamine-containing carbohydrate chains, or galactose-containing carbohydrate chains), and lipid moieties (e.g., cholesterol and phospholipids). Other conjugate groups include, but are not limited to phospholipids, biotin or other affinity tags (e.g., streptavidin), polymers (e.g., polyethylene glycol (PEG), polylysine), cell-penetrating peptides (e.g., Tat, Penetratin), enzymes (e.g., horseradish peroxidase, alkaline phosphatase), antibodies or antibody fragments (e.g., anti-HER2, anti-EGFR), proteins or peptides (e.g., growth factors, cytokines), metal nanoparticles (e.g., gold nanoparticles), small molecule drugs (e.g., doxorubicin, paclitaxel), phenazine, folate, phenanthridine, anthraquinone, acridine, fluoresceins, rhodamines, coumarins, and dyes

The oligonucleotides of the disclosure may also be modified to have one or more stabilizing groups that are generally attached to one or both termini of oligonucleotides to enhance properties such as, for example, nuclease stability. Included in stabilizing groups are cap structures. These terminal modifications can protect the oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

### Conjugated Oligonucleotides of the Disclosure May be Modified as Described in the Preceding Section.

### A. 5′ Wing Segment, General (W1)

In certain embodiments, at least one of the nucleosides in the 5′ wing segment comprises a 2′-O(CH₂)₂—OCH₃ sugar. In certain embodiments, each of the nucleosides in the 5′ wing segment comprises a 2′-O(CH₂)₂—OCH₃ sugar. In certain embodiments, the 5′ wing segment comprises a nucleoside with a 2′-deoxy sugar. In certain embodiments, the 5′ wing segment comprises a nucleoside with a 2′-deoxy sugar, and the other nucleosides in the 5′ wing segment comprise a 2′-O(CH₂)₂—OCH₃ sugar.

In certain embodiments, one, two, three, or four of the nucleosides in the 5′ wing segment comprise a sugar modification described herein. In certain embodiments, one, two, three, or four of the nucleosides in the 5′ wing segment comprise a bicyclic sugar. In certain embodiments, one, two, three, or four of the nucleosides in the 5′ wing segment comprise a constrained ethyl sugar.

In certain embodiments, one, two, three, or four of the nucleosides in the 5′ wing segment comprise a locked nucleic acid. In certain embodiments, one, two, three, or four of the nucleosides in the 5′ wing segment comprise a 4′-CH₂—O-2′ sugar. In certain embodiments, two of the nucleosides in the 5′ wing segment comprise a locked nucleic acid. In certain embodiments, two of the nucleosides in the 5′ wing segment comprise a 4′-CH₂—O-2′ sugar.

### B1. 5′ Wing Segment w/4 Linked Nucleosides (W1)

In certain embodiments, the 5′ wing segment consists of 4 linked nucleosides. In certain embodiments, the 5′ wing segment consists of 4 linked nucleosides, wherein each of the 4 linked nucleosides comprise a nucleoside with a 2′-O(CH₂)₂—OCH₃ sugar. In certain embodiments, the 5′ wing segment consists of 4 linked nucleosides, wherein 3 of the 4 linked nucleosides comprise a nucleoside with a 2′-O(CH₂)₂—OCH₃ sugar. In certain embodiments, the 5′ wing segment consists of 4 linked nucleosides, wherein 2 of the 4 linked nucleosides comprise a nucleoside with a 2′-O(CH₂)₂—OCH₃ sugar. In certain embodiments, the 5′ wing segment consists of 4 linked nucleosides, wherein 1 of the 4 linked nucleosides comprises a nucleoside with a 2′-O(CH₂)₂—OCH₃ sugar.

B2. 5′ Wing Segment w/4 Linked Nucleosides, 2′-deoxy Combinations (W1)

In certain embodiments, the 5′ wing segment consists of 4 linked nucleosides, wherein one of the linked nucleosides comprises a nucleoside with a 2′-deoxy sugar. In certain embodiments, the 5′ wing segment consists of 4 linked nucleosides, wherein two of the linked nucleosides comprises a nucleoside with a 2′-deoxy sugar. In certain embodiments, the 5′ wing segment consists of 4 linked nucleosides, wherein one of the 4 linked nucleosides comprises a nucleoside with a 2′-deoxy sugar, and each of the other 3 linked nucleosides comprise a nucleoside with a 2′-O(CH₂)₂—OCH₃ sugar. In certain embodiments, the 5′ wing segment consists of 4 linked nucleosides, wherein one of the 4 linked nucleosides comprises a nucleoside with a 2′-deoxy sugar, one of the 4 linked nucleosides comprises a locked nucleic acid, and each of the other 2 linked nucleosides comprise a nucleoside with a 2′-O(CH₂)₂—OCH₃ sugar. In certain embodiments, the 5′ wing segment consists of 4 linked nucleosides, wherein one of the 4 linked nucleosides comprises a nucleoside with a 2′-deoxy sugar, two of the 4 linked nucleosides comprise a locked nucleic acid, and the other linked nucleoside comprises a nucleoside with a 2′-O(CH₂)₂—OCH₃ sugar.

### B3. 5′ Wing Segment w/4 Linked Nucleosides, 2′-LNA Combinations (W1)

In certain embodiments, the 5′ wing segment consists of 4 linked nucleosides, wherein one of the 4 linked nucleosides comprise a locked nucleic acid. In certain embodiments, the 5′ wing segment consists of 4 linked nucleosides, wherein two of the 4 linked nucleosides comprise a locked nucleic acid. In certain embodiments, the 5′ wing segment consists of 4 linked nucleosides, wherein one of the 4 linked nucleosides comprise a 4′-CH₂—O-2′ sugar. In certain embodiments, the 5′ wing segment consists of 4 linked nucleosides, wherein two of the 4 linked nucleosides comprise a 4′-CH₂—O-2′ sugar.

In certain embodiments, the 5′ wing segment consists of 4 linked nucleosides, wherein one of the 4 linked nucleosides comprise a locked nucleic acid, and each of the other 3 linked nucleosides comprise a nucleoside with a 2′-O(CH₂)₂—OCH₃ sugar. In certain embodiments, the 5′ wing segment consists of 4 linked nucleosides, wherein two of the 4 linked nucleosides comprise a locked nucleic acid, and each of the other 2 linked nucleosides comprise a nucleoside with a 2′-O(CH₂)₂—OCH₃ sugar. In certain embodiments, the 5′ wing segment consists of 4 linked nucleosides, wherein one of the 4 linked nucleosides comprise a 4′-CH₂—O-2′ sugar, and each of the other 3 linked nucleosides comprise a nucleoside with a 2′-O(CH₂)₂—OCH₃ sugar. In certain embodiments, the 5′ wing segment consists of 4 linked nucleosides, wherein two of the 4 linked nucleosides comprise a 4′-CH₂—O-2′ sugar, and each of the other 2 linked nucleosides comprise a nucleoside with a 2′-O(CH₂)₂—OCH₃ sugar.

### C1. 5′ Wing Segment w/5 Linked Nucleosides (W1)

In certain embodiments, the 5′ wing segment consists of 5 linked nucleosides. In certain embodiments, the 5′ wing segment consists of 5 linked nucleosides, wherein each of the 5 linked nucleosides comprise a nucleoside with a 2′-O(CH₂)₂—OCH₃ sugar. In certain embodiments, the 5′ wing segment consists of 5 linked nucleosides, wherein 4 of the 5 linked nucleosides comprise a nucleoside with a 2′-O(CH₂)₂—OCH₃ sugar. In certain embodiments, the 5′ wing segment consists of 5 linked nucleosides, wherein 3 of the 5 linked nucleosides comprise a nucleoside with a 2′-O(CH₂)₂—OCH₃ sugar. In certain embodiments, the 5′ wing segment consists of 5 linked nucleosides, wherein 2 of the 5 linked nucleosides comprises a nucleoside with a 2′-O(CH₂)₂—OCH₃ sugar. In certain embodiments, the 5′ wing segment consists of 5 linked nucleosides, wherein 1 of the 5 linked nucleosides comprises a nucleoside with a 2′-O(CH₂)₂—OCH₃ sugar.

C2. 5′ Wing Segment w/5 Linked Nucleosides, 2′-deoxy Combinations (W1)

In certain embodiments, the 5′ wing segment consists of 5 linked nucleosides, wherein one of the linked nucleosides comprises a nucleoside with a 2′-deoxy sugar. In certain embodiments, the 5′ wing segment consists of 5 linked nucleosides, wherein two of the linked nucleosides comprises a nucleoside with a 2′-deoxy sugar. In certain embodiments, the 5′ wing segment consists of 5 linked nucleosides, wherein one of the 5 linked nucleosides comprises a nucleoside with a 2′-deoxy sugar, and each of the other 4 linked nucleosides comprise a nucleoside with a 2′-O(CH₂)₂—OCH₃ sugar. In certain embodiments, the 5′ wing segment consists of 5 linked nucleosides, wherein one of the 5 linked nucleosides comprises a nucleoside with a 2′-deoxy sugar, one of the 5 linked nucleosides comprises a locked nucleic acid, and each of the other 3 linked nucleosides comprise a nucleoside with a 2′-O(CH₂)₂—OCH₃ sugar. In certain embodiments, the 5′ wing segment consists of 5 linked nucleosides, wherein one of the 5 linked nucleosides comprises a nucleoside with a 2′-deoxy sugar, two of the 5 linked nucleosides comprise a locked nucleic acid, and the other 2 linked nucleosides comprises a nucleoside with a 2′-O(CH₂)₂—OCH₃ sugar.

### C3. 5′ Wing Segment w/5 Linked Nucleosides, 2′-LNA Combinations (W1)

In certain embodiments, the 5′ wing segment consists of 5 linked nucleosides, wherein one of the 5 linked nucleosides comprise a locked nucleic acid. In certain embodiments, the 5′ wing segment consists of 5 linked nucleosides, wherein two of the 5 linked nucleosides comprise a locked nucleic acid. In certain embodiments, the 5′ wing segment consists of 5 linked nucleosides, wherein one of the 5 linked nucleosides comprise a 4′-CH₂—O-2′ sugar. In certain embodiments, the 5′ wing segment consists of 5 linked nucleosides, wherein two of the 5 linked nucleosides comprise a 4′-CH₂—O-2′ sugar.

In certain embodiments, the 5′ wing segment consists of 5 linked nucleosides, wherein one of the 5 linked nucleosides comprise a locked nucleic acid, and each of the other 4 linked nucleosides comprise a nucleoside with a 2′-O(CH₂)₂—OCH₃ sugar. In certain embodiments, the 5′ wing segment consists of 5 linked nucleosides, wherein two of the 5 linked nucleosides comprise a locked nucleic acid, and each of the other 3 linked nucleosides comprise a nucleoside with a 2′-O(CH₂)₂—OCH₃ sugar. In certain embodiments, the 5′ wing segment consists of 5 linked nucleosides, wherein one of the 5 linked nucleosides comprise a 4′-CH₂—O-2′ sugar, and each of the other 4 linked nucleosides comprise a nucleoside with a 2′-O(CH₂)₂—OCH₃ sugar. In certain embodiments, the 5′ wing segment consists of 5 linked nucleosides, wherein two of the 5 linked nucleosides comprise a 4′-CH₂—O-2′ sugar, and each of the other 3 linked nucleosides comprise a nucleoside with a 2′-O(CH₂)₂—OCH₃ sugar.

### D. 3′ Wing Segment, General (W2)

In certain embodiments, at least one of the nucleosides in the 3′ wing segment comprises a 2′-O(CH₂)₂—OCH₃ sugar. In certain embodiments, each of the nucleosides in the 3′ wing segment comprise a 2′-O(CH₂)₂—OCH₃ sugar. In certain embodiments, the 3′ wing segment comprises a nucleoside with a 2′-deoxy sugar. In certain embodiments, the 3′ wing segment comprises a nucleoside with a 2′-deoxy sugar, and the other nucleosides in the 3′ wing segment comprise a 2′-O(CH₂)₂—OCH₃ sugar. In certain embodiments, the 3′ wing segment further comprises two nucleosides each with a 2′-deoxy sugar.

In certain embodiments, one, two, three, or four of the nucleosides in the 3′ wing segment comprise a sugar modification described herein. In certain embodiments, one, two, three, or four of the nucleosides in the 3′ wing segment comprise a bicyclic sugar. In certain embodiments, one, two, three, or four of the nucleosides in the 3′ wing segment comprise a constrained ethyl sugar.

In certain embodiments, one, two, three, or four of the nucleosides in the 3′ wing segment comprise a locked nucleic acid. In certain embodiments, one, two, three, or four of the nucleosides in the 3′ wing segment comprise a 4′-CH₂—O-2′ sugar. In certain embodiments, two of the nucleosides in the 3′ wing segment comprise a locked nucleic acid. In certain embodiments, two of the nucleosides in the 3′ wing segment comprise a 4′-CH₂—O-2′ sugar. In certain embodiments, three of the nucleosides in the 3′ wing segment comprise a locked nucleic acid. In certain embodiments, three of the nucleosides in the 3′ wing segment comprise a 4′-CH₂—O-2′ sugar.

### E1. 3′ Wing Segment w/4 Linked Nucleosides (W2)

In certain embodiments, the 3′ wing segment consists of 4 linked nucleosides. In certain embodiments, the 3′ wing segment consists of 4 linked nucleosides, wherein each of the 4 linked nucleosides comprise a nucleoside with a 2′-O(CH₂)₂—OCH₃ sugar. In certain embodiments, the 3′ wing segment consists of 4 linked nucleosides, wherein 3 of the 4 linked nucleosides comprise a nucleoside with a 2′-O(CH₂)₂—OCH₃ sugar. In certain embodiments, the 3′ wing segment consists of 4 linked nucleosides, wherein 2 of the 4 linked nucleosides comprise a nucleoside with a 2′-O(CH₂)₂—OCH₃ sugar. In certain embodiments, the 3′ wing segment consists of 4 linked nucleosides, wherein 1 of the 4 linked nucleosides comprises a nucleoside with a 2′-O(CH₂)₂—OCH₃ sugar.

E2. 3′ Wing Segment w/4 Linked Nucleosides, 2′-deoxy Combinations (W2)

In certain embodiments, the 3′ wing segment consists of 4 linked nucleosides, wherein one of the linked nucleosides comprises a nucleoside with a 2′-deoxy sugar. In certain embodiments, the 3′ wing segment consists of 4 linked nucleosides, wherein two of the linked nucleosides comprises a nucleoside with a 2′-deoxy sugar. In certain embodiments, the 3′ wing segment consists of 4 linked nucleosides, wherein one of the 4 linked nucleosides comprises a nucleoside with a 2′-deoxy sugar, and each of the other 3 linked nucleosides comprise a nucleoside with a 2′-O(CH₂)₂—OCH₃ sugar. In certain embodiments, the 3′ wing segment consists of 4 linked nucleosides, wherein one of the 4 linked nucleosides comprises a nucleoside with a 2′-deoxy sugar, one of the 4 linked nucleosides comprises a locked nucleic acid, and each of the other 2 linked nucleosides comprise a nucleoside with a 2′-O(CH₂)₂—OCH₃ sugar. In certain embodiments, the 3′ wing segment consists of 4 linked nucleosides, wherein one of the 4 linked nucleosides comprises a nucleoside with a 2′-deoxy sugar, two of the 4 linked nucleosides comprise a locked nucleic acid, and the other linked nucleoside comprises a nucleoside with a 2′-O(CH₂)₂—OCH₃ sugar.

### E3. 3′ Wing Segment w/4 Linked Nucleosides, 2′-LNA Combinations (W2)

In certain embodiments, the 3′ wing segment consists of 4 linked nucleosides, wherein one of the 4 linked nucleosides comprise a locked nucleic acid. In certain embodiments, the 3′ wing segment consists of 4 linked nucleosides, wherein two of the 4 linked nucleosides comprise a locked nucleic acid. In certain embodiments, the 3′ wing segment consists of 4 linked nucleosides, wherein one of the 4 linked nucleosides comprise a 4′-CH₂—O-2′ sugar. In certain embodiments, the 3′ wing segment consists of 4 linked nucleosides, wherein two of the 4 linked nucleosides comprise a 4′-CH₂—O-2′ sugar.

In certain embodiments, the 3′ wing segment consists of 4 linked nucleosides, wherein one of the 4 linked nucleosides comprise a locked nucleic acid, and each of the other 3 linked nucleosides comprise a nucleoside with a 2′-O(CH₂)₂—OCH₃ sugar. In certain embodiments, the 3′ wing segment consists of 4 linked nucleosides, wherein two of the 4 linked nucleosides comprise a locked nucleic acid, and each of the other 2 linked nucleosides comprise a nucleoside with a 2′-O(CH₂)₂—OCH₃ sugar. In certain embodiments, the 3′ wing segment consists of 4 linked nucleosides, wherein three of the 4 linked nucleosides comprise a locked nucleic acid, and the other linked nucleoside comprises a nucleoside with a 2′-O(CH₂)₂—OCH₃ sugar. In certain embodiments, the 3′ wing segment consists of 4 linked nucleosides, wherein one of the 4 linked nucleosides comprise a 4′-CH₂—O-2′ sugar, and each of the other 3 linked nucleosides comprise a nucleoside with a 2′-O(CH₂)₂—OCH₃ sugar. In certain embodiments, the 3′ wing segment consists of 4 linked nucleosides, wherein two of the 4 linked nucleosides comprise a 4′-CH₂—O-2′ sugar, and each of the other 2 linked nucleosides comprise a nucleoside with a 2′-O(CH₂)₂—OCH₃ sugar. In certain embodiments, the 3′ wing segment consists of 4 linked nucleosides, wherein three of the 4 linked nucleosides comprise a 4′-CH₂—O-2′ sugar, and the other linked nucleoside comprises a nucleoside with a 2′-O(CH₂)₂—OCH₃ sugar.

### F1. 3′ Wing Segment w/5 Linked Nucleosides (W2)

In certain embodiments, the 3′ wing segment consists of 5 linked nucleosides. In certain embodiments, the 3′ wing segment consists of 5 linked nucleosides, wherein each of the 5 linked nucleosides comprise a nucleoside with a 2′-O(CH₂)₂—OCH₃ sugar. In certain embodiments, the 3′ wing segment consists of 5 linked nucleosides, wherein 4 of the 5 linked nucleosides comprise a nucleoside with a 2′-O(CH₂)₂—OCH₃ sugar. In certain embodiments, the 3′ wing segment consists of 5 linked nucleosides, wherein 3 of the 5 linked nucleosides comprise a nucleoside with a 2′-O(CH₂)₂—OCH₃ sugar. In certain embodiments, the 3′ wing segment consists of 5 linked nucleosides, wherein 2 of the 5 linked nucleosides comprises a nucleoside with a 2′-O(CH₂)₂—OCH₃ sugar. In certain embodiments, the 3′ wing segment consists of 5 linked nucleosides, wherein 1 of the 5 linked nucleosides comprises a nucleoside with a 2′-O(CH₂)₂—OCH₃ sugar.

F2. 3′ Wing Segment w/5 Linked Nucleosides, 2′-deoxy Combinations (W2)

In certain embodiments, the 3′ wing segment consists of 5 linked nucleosides, wherein one of the linked nucleosides comprises a nucleoside with a 2′-deoxy sugar. In certain embodiments, the 3′ wing segment consists of 5 linked nucleosides, wherein two of the linked nucleosides comprises a nucleoside with a 2′-deoxy sugar. In certain embodiments, the 3′ wing segment consists of 5 linked nucleosides, wherein one of the 5 linked nucleosides comprises a nucleoside with a 2′-deoxy sugar, and each of the other 4 linked nucleosides comprise a nucleoside with a 2′-O(CH₂)₂—OCH₃ sugar. In certain embodiments, the 3′ wing segment consists of 5 linked nucleosides, wherein one of the 5 linked nucleosides comprises a nucleoside with a 2′-deoxy sugar, one of the 5 linked nucleosides comprises a locked nucleic acid, and each of the other 3 linked nucleosides comprise a nucleoside with a 2′-O(CH₂)₂—OCH₃ sugar. In certain embodiments, the 3′ wing segment consists of 5 linked nucleosides, wherein one of the 5 linked nucleosides comprises a nucleoside with a 2′-deoxy sugar, two of the 5 linked nucleosides comprise a locked nucleic acid, and the other 2 linked nucleosides comprises a nucleoside with a 2′-O(CH₂)₂—OCH₃ sugar.

### F3. 3′ Wing Segment w/5 Linked Nucleosides, 2′-LNA Combinations (W2)

In certain embodiments, the 3′ wing segment consists of 5 linked nucleosides, wherein one of the 5 linked nucleosides comprise a locked nucleic acid. In certain embodiments, the 3′ wing segment consists of 5 linked nucleosides, wherein two of the 5 linked nucleosides comprise a locked nucleic acid. In certain embodiments, the 3′ wing segment consists of 5 linked nucleosides, wherein one of the 5 linked nucleosides comprise a 4′-CH₂—O-2′ sugar. In certain embodiments, the 3′ wing segment consists of 5 linked nucleosides, wherein two of the 5 linked nucleosides comprise a 4′-CH₂—O-2′ sugar.

In certain embodiments, the 3′ wing segment consists of 5 linked nucleosides, wherein one of the 5 linked nucleosides comprise a locked nucleic acid, and each of the other 4 linked nucleosides comprise a nucleoside with a 2′-O(CH₂)₂—OCH₃ sugar. In certain embodiments, the 3′ wing segment consists of 5 linked nucleosides, wherein two of the 5 linked nucleosides comprise a locked nucleic acid, and each of the other 3 linked nucleosides comprise a nucleoside with a 2′-O(CH₂)₂—OCH₃ sugar. In certain embodiments, the 3′ wing segment consists of 5 linked nucleosides, wherein three of the 5 linked nucleosides comprise a locked nucleic acid, and each of the other 2 linked nucleosides comprise a nucleoside with a 2′-O(CH₂)₂—OCH₃ sugar. In certain embodiments, the 3′ wing segment consists of 5 linked nucleosides, wherein one of the 5 linked nucleosides comprise a 4′-CH₂—O-2′ sugar, and each of the other 4 linked nucleosides comprise a nucleoside with a 2′-O(CH₂)₂—OCH₃ sugar. In certain embodiments, the 3′ wing segment consists of 5 linked nucleosides, wherein two of the 5 linked nucleosides comprise a 4′-CH₂—O-2′ sugar, and each of the other 3 linked nucleosides comprise a nucleoside with a 2′-O(CH₂)₂—OCH₃ sugar

### G1. 3′ Wing Segment w/6 Linked Nucleosides (W2)

In certain embodiments, the 3′ wing segment consists of 6 linked nucleosides. In certain embodiments, the 3′ wing segment consists of 6 linked nucleosides, wherein each of the 6 linked nucleosides comprise a nucleoside with a 2′-O(CH₂)₂—OCH₃ sugar. In certain embodiments, the 3′ wing segment consists of 6 linked nucleosides, wherein 5 of the 6 linked nucleosides comprise a nucleoside with a 2′-O(CH₂)₂—OCH₃ sugar. In certain embodiments, the 3′ wing segment consists of 6 linked nucleosides, wherein 4 of the 6 linked nucleosides comprise a nucleoside with a 2′-O(CH₂)₂—OCH₃ sugar. In certain embodiments, the 3′ wing segment consists of 6 linked nucleosides, wherein 3 of the 6 linked nucleosides comprise a nucleoside with a 2′-O(CH₂)₂—OCH₃ sugar. In certain embodiments, the 3′ wing segment consists of 6 linked nucleosides, wherein 2 of the 6 linked nucleosides comprises a nucleoside with a 2′-O(CH₂)₂—OCH₃ sugar. In certain embodiments, the 3′ wing segment consists of 6 linked nucleosides, wherein 1 of the 6 linked nucleosides comprises a nucleoside with a 2′-O(CH₂)₂—OCH₃ sugar.

G2. 3′ Wing Segment w/6 Linked Nucleosides, 2′-deoxy Combinations (W2)

In certain embodiments, the 3′ wing segment consists of 6 linked nucleosides, wherein one of the linked nucleosides comprises a nucleoside with a 2′-deoxy sugar. In certain embodiments, the 3′ wing segment consists of 6 linked nucleosides, wherein two of the linked nucleosides comprises a nucleoside with a 2′-deoxy sugar. In certain embodiments, the 3′ wing segment consists of 6 linked nucleosides, wherein one of the 6 linked nucleosides comprises a nucleoside with a 2′-deoxy sugar, and each of the other 5 linked nucleosides comprise a nucleoside with a 2′-O(CH₂)₂—OCH₃ sugar. In certain embodiments, the 3′ wing segment consists of 6 linked nucleosides, wherein one of the 6 linked nucleosides comprises a nucleoside with a 2′-deoxy sugar, one of the 6 linked nucleosides comprises a locked nucleic acid, and each of the other 4 linked nucleosides comprise a nucleoside with a 2′-O(CH₂)₂—OCH₃ sugar. In certain embodiments, the 3′ wing segment consists of 6 linked nucleosides, wherein one of the 5 linked nucleosides comprises a nucleoside with a 2′-deoxy sugar, two of the 6 linked nucleosides comprise a locked nucleic acid, and the other 3 linked nucleosides comprises a nucleoside with a 2′-O(CH₂)₂—OCH₃ sugar.

### G3. 3′ Wing Segment w/6 Linked Nucleosides, 2′-LNA Combinations (W2)

In certain embodiments, the 3′ wing segment consists of 6 linked nucleosides, wherein one of the 6 linked nucleosides comprise a locked nucleic acid. In certain embodiments, the 3′ wing segment consists of 6 linked nucleosides, wherein two of the 6 linked nucleosides comprise a locked nucleic acid. In certain embodiments, the 3′ wing segment consists of 6 linked nucleosides, wherein one of the 6 linked nucleosides comprise a 4′-CH₂—O-2′ sugar. In certain embodiments, the 3′ wing segment consists of 6 linked nucleosides, wherein two of the 6 linked nucleosides comprise a 4′-CH₂—O-2′ sugar.

In certain embodiments, the 3′ wing segment consists of 6 linked nucleosides, wherein one of the 6 linked nucleosides comprise a locked nucleic acid, and each of the other 5 linked nucleosides comprise a nucleoside with a 2′-O(CH₂)₂—OCH₃ sugar. In certain embodiments, the 3′ wing segment consists of 6 linked nucleosides, wherein two of the 6 linked nucleosides comprise a locked nucleic acid, and each of the other 4 linked nucleosides comprise a nucleoside with a 2′-O(CH₂)₂—OCH₃ sugar. In certain embodiments, the 3′ wing segment consists of 6 linked nucleosides, wherein three of the 6 linked nucleosides comprise a locked nucleic acid, and each of the other 3 linked nucleosides comprise a nucleoside with a 2′-O(CH₂)₂—OCH₃ sugar. In certain embodiments, the 3′ wing segment consists of 6 linked nucleosides, wherein one of the 6 linked nucleosides comprise a 4′-CH₂—O-2′ sugar, and each of the other 5 linked nucleosides comprise a nucleoside with a 2′-O(CH₂)₂—OCH₃ sugar. In certain embodiments, the 3′ wing segment consists of 6 linked nucleosides, wherein two of the 6 linked nucleosides comprise a 4′-CH₂—O-2′ sugar, and each of the other 4 linked nucleosides comprise a nucleoside with a 2′-O(CH₂)₂—OCH₃ sugar. In certain embodiments, the 3′ wing segment consists of 6 linked nucleosides, wherein three of the 6 linked nucleosides comprise a 4′-CH₂—O-2′ sugar, and each of the other 3 linked nucleosides comprise a nucleoside with a 2′-O(CH₂)₂—OCH₃ sugar.

### H-0. Gap Segments

In some embodiments, a modified multi-segmented antisense oligonucleotide comprises or consists of at least one gap segment. In some embodiments, a modified multi-segmented antisense oligonucleotide comprises or consists of at least two gap segments. In some embodiments, a modified multi-segmented antisense oligonucleotide comprises or consists of at least three gap segments. In some embodiments, a modified multi-segmented antisense oligonucleotide comprises 1, 2, 3, 4, 5, 6, 7, 8, or 9 or 10 gap segments. Gap segments are consecutively referred to a first, a second, a third, a fourth, a fifth, a sixth, a seventh, an eight, a nineth or a tenth gap segment, respectively, with the first gap segment being nearest to the 5′ end of the modified multi-segmented antisense oligonucleotide and last gap segment being nearest to the 3′ end of the modified multi-segmented antisense oligonucleotide. In some embodiments, a modified multi-segmented antisense oligonucleotide comprises or consists of 2 gap segments. In some embodiments, a modified multi-segmented antisense oligonucleotide comprises or consists of 3 gap segments. In some embodiments, a modified multi-segmented antisense oligonucleotide comprises or consists of 4 gap segments. In some embodiments, a modified multi-segmented antisense oligonucleotide comprises or consists of 5 gap segments. In some embodiments, a modified multi-segmented antisense oligonucleotide comprises or consists of 6 gap segments. In some embodiments, a modified multi-segmented antisense oligonucleotide comprises or consists of 7 gap segments.

In some embodiments, a gap segment comprises or consists of 1 to 20 linked nucleosides. In some embodiments, a gap segment comprises or consists of 2 to 7 linked nucleosides. In some embodiments, the gap segment consists of 1 nucleoside. In some embodiments, the gap segment consists of 2 linked nucleosides. In some embodiments, the gap segment consists of 3 linked nucleosides. In some embodiments, the gap segment consists of 4 linked nucleosides. In some embodiments, the gap segment consists of 5 linked nucleosides. In some embodiments, the gap segment consists of 6 linked nucleosides. In some embodiments, the gap segment consists of 7 linked nucleosides. In some embodiments, the gap segment consists of 8 linked nucleosides. In some embodiments, the gap segment consists of 9 linked nucleosides. In some embodiments, the gap segment consists of 10 linked nucleosides.

### H-1. First Gap Segment (G1)

In certain embodiments, the first gap segment comprises or consists of 1, 2, 3, 4, 5, 6, 7, or 8 linked nucleosides. In certain embodiments, the first gap segment comprises or consists of 2 to 7 linked nucleosides. In certain embodiments, the first gap segment comprises or consists of 4 or 5 linked nucleosides. In certain embodiments, the first gap segment comprises or consists of 4 linked nucleosides. In certain embodiments, the first gap segment comprises or consists of 5 linked nucleosides.

In certain embodiments, the first gap segment comprises or consists of 1, 2, 3, 4, 5, 6, 7, or 8 linked nucleosides, each with 2′-deoxy sugars. In certain embodiments, the first gap segment comprises or consists of 2 to 7 linked nucleosides, each with 2′-deoxy sugars. In certain embodiments, the first gap segment comprises or consists of 4 or 5 linked nucleosides, each with 2′-deoxy sugars. In certain embodiments, the first gap segment comprises or consists of 4 linked nucleosides, each with 2′-deoxy sugars. In certain embodiments, the first gap segment comprises or consists of 5 linked nucleosides, each with 2′-deoxy sugars.

### H-2. Second Gap Segment (G2)

In certain embodiments, the second gap segment comprises or consists of 1, 2, 3, 4, 5, 6, 7, or 8 linked nucleosides. In certain embodiments, the second gap segment comprises or consists of 2 to 7 linked nucleosides. In certain embodiments, the second gap segment comprises or consists of 1 or 2 linked nucleosides. In certain embodiments, the second gap segment comprises or consists of 4 or 5 linked nucleosides. In certain embodiments, the second gap segment comprises 1 nucleoside. In certain embodiments the second gap comprises or consists of 2 linked nucleosides. In certain embodiments, the second gap segment comprises or consists of 4 linked nucleosides. In certain embodiments, the second gap segment comprises or consists of 5 linked nucleosides.

In certain embodiments, the second gap segment comprises or consists of 1, 2, 3, 4, 5, 6, 7, or 8 linked nucleosides, each with 2′-deoxy sugars. In certain embodiments, the second gap segment comprises or consists of 2 to 7 linked nucleosides, each with 2′-deoxy sugars. In certain embodiments, the second gap segment comprises or consists of 1 or 2 linked nucleosides, each with 2′-deoxy sugars. In certain embodiments, the second gap segment comprises 1 nucleoside, with a 2′-deoxy sugar. In certain embodiments the second gap comprises or consists of 2 linked nucleosides, each with 2′-deoxy sugars. In certain embodiments, the second gap segment comprises or consists of 4 linked nucleosides, each with 2′-deoxy sugars. In certain embodiments, the second gap segment comprises or consists of 5 linked nucleosides, each with 2′-deoxy sugars. In certain embodiments, the second gap segment comprises or consists of 6 linked nucleosides, each with 2′-deoxy sugars.

### H-3. Third Gap Segment (G3)

In certain embodiments, the third gap segment comprises or consists of 1, 2, 3, 4, 5, 6, 7, or 8 linked nucleosides. In certain embodiments, the third gap segment comprises or consists of 2 to 7 linked nucleosides. In certain embodiments, the third gap segment comprises or consists of 4 or 5 linked nucleosides. In certain embodiments, the third gap segment comprises or consists of 4 linked nucleosides. In certain embodiments, the third gap segment comprises or consists of 5 linked nucleosides.

In certain embodiments, the third gap segment comprises or consists of 1, 2, 3, 4, 5, 6, 7, or 8 linked nucleosides, each with 2′-deoxy sugars. In certain embodiments, the third gap segment comprises or consists of 2 to 7 linked nucleosides, each with 2′-deoxy sugars. In certain embodiments, the third gap segment comprises or consists of 4 or 5 linked nucleosides, each with 2′-deoxy sugars. In certain embodiments, the third gap segment comprises or consists of 4 linked nucleosides, each with 2′-deoxy sugars. In certain embodiments, the third gap segment comprises or consists of 5 linked nucleosides, each with 2′-deoxy sugars.

### J. Separator Segment

In some embodiments, the separator segment comprises 1, 2, 3, 4 or 5 linked nucleosides. In some embodiments, the separator segment comprises 1 nucleoside. In some embodiments, the separator segment comprises 2 nucleosides.

In some embodiments, the nucleoside comprises a modification. Exemplary modifications include any one of the modifications shown in the section titled Exemplary Modifications, including but not limited to a 2′-O-methoxyethyl nucleoside, 2′-O-methoxyethyl cytidine, 2′-O-methyl nucleoside, 2′-O-methyl nucleoside, 2′-OH nucleoside, GNA, LNA, LNA with cytosine base, 2′-Fluoro 2′-deoxy nucleoside, 2′-Fluoro 2′-deoxy cytidine, 2′-F-Arabinonucleic Acid (2′-F-ANA), or 2′-F-Arabinonucleic Acid (2′-F-ANA), or a combination thereof.

In certain embodiments, the separator segment consists of one nucleoside comprising a 2′-OCH₃ sugar. In certain embodiments, the separator segment consists of one nucleoside comprising a 2′-O(CH₂)₂—OCH₃ sugar.

In certain embodiments, the separator segment is at position 7, 8, 9, 10, 11, 12, 13, or 14 of a modified multi-segmented antisense oligonucleotide that is 16 residues in length. In certain embodiments, the separator segment is at position 7, 8, 9, 10, 11, 12, 13, or 14 of a modified multi-segmented antisense oligonucleotide that is 17 residues in length. In certain embodiments, the separator segment is at position 7, 8, 9, 10, 11, 12, 13, or 14 of a modified multi-segmented antisense oligonucleotide that is 18 residues in length. In certain embodiments, the separator segment is at position 7, 8, 9, 10, 11, 12, 13, or 14 of a modified multi-segmented antisense oligonucleotide that is 19 residues in length. In certain embodiments, the separator segment is at position 7, 8, 9, 10, 11, 12, 13, or 14 of a modified multi-segmented antisense oligonucleotide that is 20 residues in length. In certain embodiments, the separator segment is at position 7, 8, 9, 10, 11, 12, 13, or 14 of a modified multi-segmented antisense oligonucleotide that is 21 residues in length. In certain embodiments, the separator segment is at position 7, 8, 9, 10, 11, 12, 13, or 14 of a modified multi-segmented antisense oligonucleotide that is 22 residues in length.

In certain embodiments, the separator segment is at position 9, 10, 11 or 12 of a modified multi-segmented antisense oligonucleotide that is 20 residues in length.

In certain embodiments, the separator segment is at position 10 of a modified multi-segmented antisense oligonucleotide that is 20 residues in length.

In certain embodiments, the separator segment is at position 10 of a modified multi-segmented antisense oligonucleotide that is 20 residues in length and consists of one nucleoside comprising a 2′-O(CH₂)₂—OCH₃ sugar. In certain embodiments, the separator segment consists of one nucleoside comprising a 2′-OCH₃ sugar. In certain embodiments, the separator segment is at position 7 or 8 or 9 or 10 or 11 or 12 or 13 or 14 of a modified multi-segmented antisense oligonucleotide that is 20 residues in length. In certain embodiments, the separator segment is at position 9 or 10 or 11 of a modified multi-segmented antisense oligonucleotide that is 20 residues in length. In certain embodiments, the separator segment is at position 10 of a modified multi-segmented antisense oligonucleotide that is 20 residues in length.

In certain embodiments, the separator segment consists of one nucleoside comprising a 2′-O(CH₂)₂—OCH₃ sugar. In certain embodiments, the separator segment consists of one nucleoside comprising a 2′-OCH₃ sugar. In certain embodiments, the separator segment is at position 7 or 8 or 9 or 10 or 11 or 12 or 13 or 14 of a modified multi-segmented antisense oligonucleotide that is 20 residues in length. In certain embodiments, the separator segment is at position 9 or 10 or 11 of a modified multi-segmented antisense oligonucleotide that is 20 residues in length. In certain embodiments, the separator segment is at position 10 of a modified multi-segmented antisense oligonucleotide that is 20 residues in length.

### K. Oligonucleotide Backbone

In certain embodiments, the modified multi-segmented antisense oligonucleotide is a single-stranded. In certain embodiments, at least one internucleoside linkage is a modified internucleoside linkage. In certain embodiments, each internucleoside linkage is a phosphorothioate internucleoside linkage. In some embodiments, a modified multi-segmented antisense oligonucleotide of the disclosure comprises one or more modified internucleoside linkages, wherein the modified internucleoside linkage is a phosphorodiamidate linkage between morpholino nucleoside mimetics.

### L. Modified Nucleobases

In certain embodiments, the modified multi-segmented antisense oligonucleotide comprises at least one modified nucleobase. In certain embodiments, wherein the modified multi-segmented antisense oligonucleotide comprises one, two, or three modified nucleobase(s). In certain embodiments, the modified nucleobase is a 5-methylcytosine.

In certain embodiments, one or more modified nucleosides in the wing segment have a modified sugar. In certain embodiments, the modified sugar is a bicyclic sugar. In certain embodiments, the modified nucleoside is an LNA nucleoside. In certain embodiments, the modified nucleoside is a 2′-substituted nucleoside. In certain embodiments, 2′ substituted nucleosides include nucleosides with bicyclic sugar modifications. In certain embodiments, the modified nucleoside is a 2′-MOE nucleoside. In certain embodiments, the modified nucleoside is a constrained ethyl (cEt) nucleoside. In certain embodiments, each modified nucleoside in each wing segment is independently a 2′-MOE nucleoside or a nucleoside with a bicyclic sugar modification such as a constrained ethyl (cEt) nucleoside or LNA nucleoside.

### 4-5-1-5-5 Modified Multi-Segmented Antisense Oligonucleotides

In certain embodiments, the modified multi-segmented antisense oligonucleotide comprises: a 5′ wing segment consisting of 4 linked nucleosides; a first gap segment consisting of 5 linked nucleosides with 2′-deoxy sugars; a separator segment at position 10 of a modified multi-segmented antisense oligonucleotide that is 20 residues in length; a second gap segment consisting of 5 linked nucleosides with 2′-deoxy sugars; and a 3′ wing segment consisting of 5 linked nucleosides. In an exemplary embodiment, the modified multi-segmented antisense oligonucleotide of this paragraph further comprises that the cytosines at positions 2, 13, and 20 of a modified multi-segmented antisense oligonucleotide that is 20 residues in length are each 5-methylcytosine. In an exemplary embodiment of the modified multi-segmented antisense oligonucleotide of this paragraph, each cytosine of the modified multi-segmented antisense oligonucleotide is a 5-methylcytosine. In an exemplary embodiment, the modified multi-segmented antisense oligonucleotide of this paragraph further comprises internucleoside linkages in which each is a phosphorothioate internucleoside linkage. In an exemplary embodiment, the modified multi-segmented antisense oligonucleotide of this paragraph further comprises that a cytosine at positions 2, 13, and 20 of a modified multi-segmented antisense oligonucleotide that is 20 residues in length are each 5-methylcytosine and further comprises internucleoside linkages in which each is a phosphorothioate internucleoside linkage.

In certain embodiments, the modified multi-segmented antisense oligonucleotide comprises: a 5′ wing segment described in Section B1, B2, or B3; a first gap segment consisting of 5 linked nucleosides with 2′-deoxy sugars; a separator segment at position 10 of a modified multi-segmented antisense oligonucleotide that is 20 residues in length; a second gap segment consisting of 5 linked nucleosides with 2′-deoxy sugars; and a 3′ wing segment described in Section F1, F2, or F3. In an exemplary embodiment, the modified multi-segmented antisense oligonucleotide of this paragraph further comprises that the cytosines at positions 2, 13, and 20 of a modified multi-segmented antisense oligonucleotide that is 20 residues in length are each 5-methylcytosine. In an exemplary embodiment, the modified multi-segmented antisense oligonucleotide of this paragraph further comprises internucleoside linkages in which each is a phosphorothioate internucleoside linkage. In an exemplary embodiment, the modified multi-segmented antisense oligonucleotide of this paragraph further comprises a cytosine at positions 2, 13, and 20 of a modified multi-segmented antisense oligonucleotide that is 20 residues in length are each 5-methylcytosine and further comprises internucleoside linkages in which each is a phosphorothioate internucleoside linkage.

Exemplary annotations of the positions and segments of AUS 1493 or SEQ ID NO: 456 is shown below.

| | | 5′ Wing | | | | First Gap | | | | | Separator | | | | | | | | | | |
|---|---|---|---|---|---|---|---|---|---|---|---|---|---|---|---|---|---|---|---|---|---|
| | | segment | | | | segment | | | | | segment | Second Gap segment | | | | | 3′ Wing segment | | | | |
| Position | | 1 | 2 | 3 | 4 | 5 | 6 | 7 | 8 | 9 | 10 | 11 | 12 | 13 | 14 | 15 | 16 | 17 | 18 | 19 | 20 |
| SEQ ID | AUS # | | | | | | | | | | | | | | | | | | | | |
| NO | | | | | | | | | | | | | | | | | | | | | |
| 456 | AUS1493 | G | C | A | G | a | g | g | t | g | A | a | g | c | g | a | A | +G | T | +G | +C |

In certain embodiments, the oligonucleotides or compositions comprise a salt of the modified multi-segmented antisense oligonucleotide.

In certain embodiments, the oligonucleotides or compositions further comprise a pharmaceutically acceptable carrier or diluent.

In certain embodiments, the compound or modified multi-segmented antisense oligonucleotide is single-stranded.

In certain embodiments, at least one nucleoside of the modified multi-segmented antisense oligonucleotide comprises a modified sugar. In certain embodiments, at least one modified sugar comprises a 2′-O-methoxyethyl group (2′-O(CH₂)₂—OCH₃). In certain embodiments, the modified sugar comprises a 2′-O—CH₃ group. In certain embodiments, the modified nucleobase is a 2′-O-methoxyethyl 5-methylcytidine.

In certain embodiments, at least one modified sugar is a bicyclic sugar. In certain embodiments, at least one modified sugar the bicyclic sugar comprises a 4′-(CH₂)—O-2′ bridge, wherein n is 1 or 2. In certain embodiments, the bicyclic sugar comprises a 4′-CH₂—O-2′ bridge. In certain embodiments, the bicyclic sugar comprises a 4′-CH(CH₃)—O-2′ bridge.

### Methods

The disclosure provides methods of treating a subject with an disease or disorder that implicates the target.

In some embodiments, the disease or disorder is a DMPK-associated disease or disorder. In some embodiments, the disease or disorder is a AAT-associated disease or disorder. In some embodiments, the disease or disorder is a TTR-associated disease or disorder. In some embodiments, the disease or disorder is a PCSK9-associated disease or disorder. In some embodiments, the disease or disorder is a ApoB-associated disease or disorder. In some embodiments, the disease or disorder is a TNF-alpha-associated disease or disorder. In some embodiments, the disease or disorder is a ApoCIII-alpha-associated disease or disorder. In some embodiments, the disease or disorder is a DMPK-associated disease or disorder. In some embodiments, the disease or disorder is a AGT-associated disease or disorder. In some embodiments, the disease or disorder is a CFB-associated disease or disorder. In some embodiments, the disease or disorder is a DGAT2-associated disease or disorder. In some embodiments, the disease or disorder is a PNPLA3-associated disease or disorder. In some embodiments, the disease or disorder is a ALAS1-associated disease or disorder. In some embodiments, the disease or disorder is a hydroxy acid oxidase 1-associated disease or disorder.

In some embodiments, the disease or disorder is Non-alcoholic fatty liver disease (NAFLD), nonalcoholic steatohepatitis (NASH), COVID19, skin aging conditions, prostate cancer, atherosclerosis, chronic kidney disease, chikungunya virus, amyotrophic lateral sclerosis, advanced malignant solid neoplasm, nonarteritic anterior ischemic optic neuropathy, HIV, immunoglobulin a nephropathy, inflammation and injury, human tenon capsule fibroblasts, antifibrotic, cystic fibrosis, Duchenne muscular dystrophy (DMD), myotonic dystrophy 1, centronuclear myopathy, facioscapulohumeral muscular dystrophy (FSHD), pancreatic cancer, solid tumor, cystic fibrosis, anti-thrombosis, blood coagulation disorder, thrombotic disorder, alexander disease, Pompe disease, HIN influenza, HBV, liver fibrosis, HPV, Huntington's disease, clear cell renal cell carcinoma, pancreatic cancer, Parkinson's disease, MERS-CoV, inflammatory bowel disease, Alport's syndrome, advanced liver cancer, muscle atrophy and mytonic dystrophy, Mycobacterium tuberculosis, rabies, hypercholesterolemia, lung fibrosis, primary or secondary liver cancer, breast cancer, RSV, spinal muscular atrophy, multiple system atrophy, cancer, Alzheimer's disease & frontotemporal degeneration, Thalassemia/low risk myelodysplastic syndrome, autoimmune, hATTR, hereditary transthyretin (ATTRv) amyloidosis, or gout.

In some embodiments, the disease or disorder is a Matla, (pro)renin receptor/(P)RR, COVID-19 5′UTR, Acc1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 150 Dystrophin, Exon 151 Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, H1N1, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2a, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF1, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, or YAP1 associated disease or disorder.

In certain embodiments, the subject is a human.

In certain embodiments, the subject is a non-human primate, e.g. a monkey, for example a cynomolgus monkey.

In certain embodiments, the subject is a rodent, such as a mouse or rat.

In some embodiments, the modified multi-segmented antisense oligonucleotides of the disclosure increase in vivo activity (as compared to a sequence of possessing the same nucleobase sequence, but not modified).

In some embodiments, the modified multi-segmented antisense oligonucleotides of the disclosure reduce result in reduced off-target binding (as compared to a sequence of possessing the same nucleobase sequence, but not modified).

In some embodiments, use of the modified multi-segmented antisense oligonucleotides of the disclosure result in reduced toxicity (as compared to a sequence of possessing the same nucleobase sequence, but not modified).

In certain embodiments, the modified multi-segmented antisense oligonucleotides or pharmaceutical compositions are designated as a first agent. In certain embodiments, the methods comprise administering a first agent and one or more second agents. In certain embodiments, the first agent and one or more second agents are co-administered. In certain embodiments the first agent and one or more second agents are co-administered sequentially or concomitantly. In certain embodiments, the first agent and one or more second agents are not co-administered.

Modified multi-segmented antisense oligonucleotides of the disclosure, and pharmaceutical compositions comprising same, can be administered to the subject by any suitable route of administration. Examples of routes of administration include parenteral, e.g., intravenous, intradermal, subcutaneous, oral (e.g., inhalation), transdermal (topical), intraocular, intramuscular, intraperitoneal (into the body cavity) and transmucosal administration.

In certain embodiments, administration comprises parenteral administration. In certain embodiments, administration comprises subcutaneous administration. In certain embodiments, administration comprises intravenous injection or infusion.

In some embodiments, EC₅₀ in cells treated with a modified multi-segmented antisense oligonucleotide was measured as a proxy for the effectiveness of the modified multi-segmented antisense oligonucleotide. In some embodiments, the EC₅₀ is about 0.1 nM to about 250 nM. In some embodiments, the EC₅₀ is less than 250 nM, less than 200 nM, less than 150 nM, less than 100 nM, less than 90 nM, less than 80 nM, less than 70 nM, less than 65 nM, less than 60 nM, less than 55 nM, less than 50 nM, less than 49 nM, less than 47 nM, less than 46 nM, less than 45 nM, less than 44 nM, less than 43 nM, less than 42 nM, less than 41 nM, less than 40 nM, less than 39 nM, less than 38 nM, less than 37 nM, less than 36 nM, less than 35 nM, less than 34 nM, less than 33 nM, less than 32 nM, less than 31 nM, less than 30 nM, less than 29 nM, less than 28 nM, less than 27 nM, less than 26 nM, less than 25 nM, less than 24 nM, less than 23 nM, less than 22 nM, less than 21 nM, less than 20 nM, less than 19 nM, less than 18 nM, less than 17 nM, less than 16 nM, less than 15 nM, less than 14 nM, less than 13 nM, less than 12 nM, less than 11 nM, less than 10 nM, less than 9 nM, less than 8 nM, less than 7 nM, less than 6 nM, less than 5 nM, less than 4 nM, less than 3 nM, less than 2 nM, less than lnM, less than 0.9 nM, less than 0.8 nM, less than 0.7 nM, less than 0.6 nM, less than 0.5 nM, less than 0.4 nM, less than 0.3 nM, less than 0.2 nM or less than 0.1 nM.

In some embodiments, the ratio of EC₅₀ in cells treated with a modified multi-segmented antisense oligonucleotide/EC₅₀ in cells treated with a reference oligonucleotide (e.g. modified antisense oligonucleotide with the same template nucleobase sequence which does not comprise a separator segment) is calculated as a measure of efficacy in reducing target protein expression levels. In some embodiments, the ratio is about 0.05 to about 250. In some embodiments, the ratio is about 0.05 to about 100. In some embodiments, the EC₅₀ ratio is less than 250, less than 150, less than 100, less than 90, less than 80, less than 70, less than 65, less than 60, less than 55, less than 50, less than 49, less than 47, less than 46, less than 45, less than 44, less than 43, less than 42, less than 41, less than 40, less than 39, less than 38, less than 37, less than 36, less than 35, less than 34, less than 33, less than 32, less than 31, less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, less than 1, less than 0.9, less than 0.8, less than 0.7, less than 0.6, less than 0.5, less than 0.4, less than 0.3, less than 0.2 or less than 0.1.

In some embodiments, the MTT CC25 value (nM) is used as a measure of cellular toxicity. In some embodiments the MTT CC25 of cells treated with the modified multi-segmented antisense oligonucleotide described herein is about 10 nM to about 250 nM. In some embodiments the MTT CC25 is greater than 10 nM, greater than 15 nM, greater than 20 nM, greater than 25 nM, greater than 30 nM, greater than 35 nM, greater than 40 nM, greater than 45 nM, greater than 50 nM, greater than 55 nM, greater than 60 nM, greater than 65 nM, greater than 70 nM, greater than 75 nM, greater than 80 nM, greater than 85 nM, greater than 90 nM, greater than 95 nM, greater than 100 nM, greater than 110 nM, greater than 120 nM, greater than 130 nM, greater than 140 nM, greater than 150 nM, greater than 160 nM, greater than 170 nM, greater than 180 nM, greater than 190 nM, greater than 200 nM, greater than 210 nM, greater than 220 nM, greater than 230 nM, greater than 240 nM or greater than 250 nM.

In some embodiments, the CCK8 CC30 (nM) is used as a measure of cellular toxicity. In some embodiments, the CCK8 CC30 of cells treated with the modified multi-segmented antisense oligonucleotide described herein is about 10 nM to about 250 nM. In some embodiments, the CCK8 CC30 is greater than 10 nM, greater than 15 nM, greater than 20 nM, greater than 25 nM, greater than 30 nM, greater than 35 nM, greater than 40 nM, greater than 45 nM, greater than 50 nM, greater than 55 nM, greater than 60 nM, greater than 65 nM, greater than 70 nM, greater than 75 nM, greater than 80 nM, greater than 85 nM, greater than 90 nM, greater than 95 nM, greater than 100 nM, greater than 110 nM, greater than 120 nM, greater than 130 nM, greater than 140 nM, greater than 150 nM, greater than 160 nM, greater than 170 nM, greater than 180 nM, greater than 190 nM, greater than 200 nM, greater than 210 nM, greater than 220 nM, greater than 230 nM, greater than 240 nM or greater than 250 nM.

In some embodiments, the ratio of MTT CC25 or CCK8 CC30 of cells treated with a modified multi-segmented antisense oligonucleotide/MTT CC25 or CCK8 CC30 of cells treated with a reference oligonucleotide (e.g. modified antisense oligonucleotide with the same template nucleobase sequence which does not comprise a separator segment) is calculated as a measure of cellular toxicity (relative cellular toxicity). In some embodiments, the ratio of MTT CC25 or CCK8 CC30 of cells treated with a modified multi-segmented antisense oligonucleotide/MTT CC25 or CCK8 CC30 of cells treated with a reference oligonucleotide (e.g. modified antisense oligonucleotide with the same template nucleobase sequence which does not comprise a separator segment) is less than 250, less than 150, less than 100, less than 90, less than 80, less than 70, less than 65, less than 60, less than 55, less than 50, less than 49, less than 47, less than 46, less than 45, less than 44, less than 43, less than 42, less than 41, less than 40, less than 39, less than 38, less than 37, less than 36, less than 35, less than 34, less than 33, less than 32, less than 31, less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, less than 1, less than 0.9, less than 0.8, less than 0.7, less than 0.6, less than 0.5, less than 0.4, less than 0.3, less than 0.2 or less than 0.1.

In some embodiments, the “C/E ratio” is used as a measure of efficacy in reducing target protein expression levels relative to cellular toxicity. In some embodiments, the C/E ratio is calculated by taking the ratio of MTT CC25 (nM) of a modified multi-segmented antisense oligonucleotide/MTT CC25 (nM) of a reference oligonucleotide (e.g., modified antisense oligonucleotide with the same template nucleobase sequence which does not comprise a separator segment) and dividing it by the ratio of EC₅₀ of a modified multi-segmented antisense oligonucleotide/EC50 of a reference oligonucleotide (e.g., modified antisense oligonucleotide with the same template nucleobase sequence which does not comprise a separator segment). In some embodiments, the C/E ratio is calculated by taking the ratio of CCK8 CC25 (nM) of a modified multi-segmented antisense oligonucleotide/CCK8 CC25 (nM) of a reference oligonucleotide (e.g., modified antisense oligonucleotide with the same template nucleobase sequence which does not comprise a separator segment) and dividing it by the ratio of EC₅₀ of a modified multi-segmented antisense oligonucleotide/EC50 of a reference oligonucleotide (e.g., modified antisense oligonucleotide with the same template nucleobase sequence which does not comprise a separator segment). In some embodiments, the C/E ratio is about 0.01 to about 50. In some embodiments, the C/E ratio is greater than 1, greater than 1.1, greater than 1.2, greater than 1.3, greater than 1.4, greater than 1.5, greater than 1.6, greater than 1.7, greater than 1.8, greater than 1.9, greater than 2.0, greater than 2.1, greater than 2.2, greater than 2.3, greater than 2.4, greater than 2.5, greater than 2.6, greater than 2.7, greater than 2.8, greater than 2.9, greater than 3.0, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 30, greater than 35, greater than 40, greater than 45, greater than 50, greater than 55, greater than 65, greater than 70, greater than 80, greater than 90 or greater than 100.

In some embodiments, the melting temperature of the modified multi-segmented antisense oligonucleotide is used as a measure of stability. In some embodiments, the melting temperature of modified multi-segmented antisense oligonucleotide described herein is at least 0.01 fold less than a reference oligonucleotide (e.g., modified antisense oligonucleotide with the same template nucleobase sequence which does not comprise a separator segment). In some embodiments, the melting temperature is about 0.01 fold, about 0.02 fold, about 0.03 fold, about 0.04 fold, about 0.05 fold, about 0.06 fold, about 0.07 fold, about 0.08 fold, about 0.09 fold, about 0.1 fold, about 0.15 fold, about 0.2 fold, about 0.25 fold, about 0.3 fold, about 0.35 fold, about 0.4 fold, about 0.45 fold, about 0.5 fold, about 0.55 fold, about 0.6 fold, about 0.65 fold, about 0.7 fold, about 0.75 fold, about 0.8 fold, about 0.85 fold, about 0.9 fold, about 0.95 fold, about 1 fold, about 1.1 fold, about 1.15 fold, about 1.2 fold, about 1.25 fold, about 1.3 fold, about 1.35 fold, about 1.4 fold, about 1.45 fold, about 1.5 fold, about 1.55 fold, about 1.6 fold, about 1.65 fold, about 1.7 fold, about 1.75 fold, about 1.8 fold, about 1.85 fold, about 1.9 fold, about 1.95 fold, about 2 fold, about 2.1 fold, about 2.15 fold, about 2.2 fold, about 2.25 fold, about 2.3 fold, about 2.35 fold, about 2.4 fold, about 2.45 fold, about 2.5 fold, about 2.55 fold, about 2.6 fold, about 2.65 fold, about 2.7 fold, about 2.75 fold, about 2.8 fold, about 2.85 fold, about 2.9 fold, about 2.95 fold, about 3 fold, about 3.1 fold, about 3.15 fold, about 3.2 fold, about 3.25 fold, about 3.3 fold, about 3.35 fold, about 3.4 fold, about 3.45 fold, about 3.5 fold, about 3.55 fold, about 3.6 fold, about 3.65 fold, about 3.7 fold, about 3.75 fold, about 3.8 fold, about 3.85 fold, about 3.9 fold, about 3.95 fold, about 4 fold, about 4.1 fold, about 4.15 fold, about 4.2 fold, about 4.25 fold, about 4.3 fold, about 4.35 fold, about 4.4 fold, about 4.45 fold, about 4.5 fold, about 4.55 fold, about 4.6 fold, about 4.65 fold, about 4.7 fold, about 4.75 fold, about 4.8 fold, about 4.85 fold, about 4.9 fold, about 4.95 fold, or about 5 fold less than a reference oligonucleotide (e.g., modified antisense oligonucleotide with the same template nucleobase sequence which does not comprise a separator segment).

In some embodiments, the ability of a modified multi-segmented antisense oligonucleotide to recruit a endonuclease (e.g. RNaseH) and degrade a template sequence is used as a measure of biological function. In some embodiments, the degradation of a template sequence by a modified multi-segmented oligonucleotide is at least 0.01 fold greater than a reference oligonucleotide (e.g., modified antisense oligonucleotide with the same template nucleobase sequence which does not comprise a separator segment). In some embodiments, the degradation of a template sequence by a modified multi-segmented oligonucleotide is about 0.01 fold, about 0.02 fold, about 0.03 fold, about 0.04 fold, about 0.05 fold, about 0.06 fold, about 0.07 fold, about 0.08 fold, about 0.09 fold, about 0.1 fold, about 0.15 fold, about 0.2 fold, about 0.25 fold, about 0.3 fold, about 0.35 fold, about 0.4 fold, about 0.45 fold, about 0.5 fold, about 0.55 fold, about 0.6 fold, about 0.65 fold, about 0.7 fold, about 0.75 fold, about 0.8 fold, about 0.85 fold, about 0.9 fold, about 0.95 fold, about 1 fold, about 1.1 fold, about 1.15 fold, about 1.2 fold, about 1.25 fold, about 1.3 fold, about 1.35 fold, about 1.4 fold, about 1.45 fold, about 1.5 fold, about 1.55 fold, about 1.6 fold, about 1.65 fold, about 1.7 fold, about 1.75 fold, about 1.8 fold, about 1.85 fold, about 1.9 fold, about 1.95 fold, about 2 fold, about 2.1 fold, about 2.15 fold, about 2.2 fold, about 2.25 fold, about 2.3 fold, about 2.35 fold, about 2.4 fold, about 2.45 fold, about 2.5 fold, about 2.55 fold, about 2.6 fold, about 2.65 fold, about 2.7 fold, about 2.75 fold, about 2.8 fold, about 2.85 fold, about 2.9 fold, about 2.95 fold, about 3 fold, about 3.1 fold, about 3.15 fold, about 3.2 fold, about 3.25 fold, about 3.3 fold, about 3.35 fold, about 3.4 fold, about 3.45 fold, about 3.5 fold, about 3.55 fold, about 3.6 fold, about 3.65 fold, about 3.7 fold, about 3.75 fold, about 3.8 fold, about 3.85 fold, about 3.9 fold, about 3.95 fold, about 4 fold, about 4.1 fold, about 4.15 fold, about 4.2 fold, about 4.25 fold, about 4.3 fold, about 4.35 fold, about 4.4 fold, about 4.45 fold, about 4.5 fold, about 4.55 fold, about 4.6 fold, about 4.65 fold, about 4.7 fold, about 4.75 fold, about 4.8 fold, about 4.85 fold, about 4.9 fold, about 4.95 fold, or about 5 fold greater than a reference oligonucleotide (e.g., modified antisense oligonucleotide with the same template nucleobase sequence which does not comprise a separator segment).

### Kits and Articles of Manufacture

The disclosure provides kits comprising the modified multi-segmented antisense oligonucleotides described herein, and pharmaceutical compositions comprising same. In some embodiments, the modified multi-segmented antisense oligonucleotide comprises a structure of any one of the oligonucleotide sequences described herein.

### Target Nucleic Acids, Target Regions and Nucleotide Sequences

The modified multi-segmented antisense oligonucleotides can be directed to any target of interest.

The targets can be any target for which a reduction in activity is desired. In some embodiments, the target is a mammalian target, e.g. a human or other animal target; directed to an oncogenic target, an inflammatory target, a metabolic target, a cardiovascular target, a neurological target, a neuropsychiatric target, or the like. In some embodiments, the target is a gene transcript (e.g., an mRNA) or an RNA that regulates the expression of a gene. Exemplary genes include but are not limited to DMPK, AAT, TTR, PCSK9, ApoB, ApoCIII, TNF-alpha, AGT, CFB, DGAT2, PNPLA3, ALAS1 or hydroxy acid oxidase 1. In some embodiments, the gene is DMPK, AAT, TTR or PCSK9.

In some embodiments, the gene is Matla, (pro)renin receptor/(P)RR, COVID-19 5′UTR, Acc1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 150 Dystrophin, Exon 151 Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, H1N1, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2a, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, or YAP1.

Exemplary nucleic acid target sequences include but are not limited to those shown in Table 8.1, 8.2 and 8.3. In some embodiments, the nucleic acid target comprises a sequence of any one of the sequences described in Table 8.1, 8.2 and 8.3. Each of the residues in Tables 8.1, 8.2 and 8.3 reflect only sequence information, without any modified nucleosides.

TABLE 8.1
Exemplary modified multi-segmented antisense oligonucleotide nucleobase
sequences and gene target sequences

| | | Modified multi- | | | |
|---|---|---|---|---|---|
| | | segmented antisense | SEQ ID | | |
| | | oligonucleotide | NO: | Gene Target | SEQ ID |
| Gene | AUS# | nucleobase sequence | | Sequence | NO: |
| PCSK9 | AUS-P | GGGCTCATAGCACATTA | 1432 | CCCGAGTATCGTGT | 1454 |
| | | TCC | | AATAGG | |
| DMPK | AUS-D | ACAGACAATAAATACCG | 1433 | ACAGACAATAAATA | 1455 |
| | | AGG | | CCGAGG | |
| TTR | AUS-T | TACAAATGGGATGCTAC | 1434 | ATGTTTACCCTACGA | 1456 |
| | | TGC | | TGACG | |
| AAT | AUS-A | ACCCAATTCAGAAGGAA | 1435 | TGGGTTAAGTCTTCC | 1457 |
| | | GGA | | TTCCT | |
| PCSK9 | AUS- | CCTTGGCCACGCCGGCA | 1436 | GGAACCGGTGCGGC | 1458 |
| | PA | TCC | | CGTAGG | |
| DMPK | AUS- | ACAGACAATAAATACCG | 1437 | TGTCTGTTATTTATG | 1459 |
| | DA | AGG | | GCTCC | |
| DMPK | AUS- | CGGAGCGGTTGTGAACT | 1438 | GCCTCGCCAACACT | 1460 |
| | DB | GGC | | TGACCG | |
| DMPK | AUS- | GCGUAGAAGGGCGUCU | 1439 | CGCATCTTCCCGCA | 1461 |
| | DC | GCCC | | GACGGG | |
| TTR | AUS- | UCUUGGTTACATGAAAU | 1440 | AGAACCAATGTACT | 1462 |
| | TA | CCC | | TTAGGG | |
| AAT | AUS- | TGTTAAACATGCCTAAA | 1441 | ACAATTTGTACGGA | 1463 |
| | AA | CGCT | | TTTGCGA | |
| ApoB | AUS- | GCCTCAGTCTGCTTCGCA | 1442 | CGGAGTCAGACGAA | 1464 |
| | AB | CC | | GCGTGG | |
| ApoCIII | AUS- | AGCTTCTTGTCCAGCTTT | 1443 | TCGAAGAACAGGTC | 1465 |
| | AC | AT | | GAAATA | |
| TNF- | AUS- | GCTGATTAGAGAGAGGT | 1444 | CGACTAATCTCTCTC | 1466 |
| alpha | TB | CCC | | CAGGG | |
| TNF- | AUS- | AAGAGGACCTGGGAGTA | 1445 | TTCTCCTGGACCCTC | 1467 |
| alpha | TC | GAT | | ATCTA | |
| AGT | AUS- | CACAAACAAGCTGGTCG | 1446 | GTGTTTGTTCGACCA | 1468 |
| | AG | GUU | | GCCAA | |
| CFB | AUS- | ATCCCACGCCCCTGTCC | 1447 | TAGGGTGCGGGGAC | 1469 |
| | CF | AGC | | AGGTCG | |
| DGAT2 | AUS- | TGCCATTTAATGAGCTTC | 1448 | ACGGTAAATTACTC | 1470 |
| | DG | AC | | GAAGTG | |
| PNPLA3 | AUS- | TACTTTATTCAATGTGGC | 1449 | ATGAAATAAGTTAC | 1471 |
| | PN | TT | | ACCGAA | |
| PCSK9 | AUS- | ACAAAAGCAAAACAGG | 1450 | TGTTTTCGTTTTGTC | 1472 |
| | PB | UCUA | | CAGAT | |
| ALAS1 | AUS- | UAAGAUGAGACACUCU | 1451 | ATTCTACTCTGTGAG | 1473 |
| | AL | UUCU | | AAAGA | |
| TTR | AUS- | AUGGAAUACUCUUGGU | 1452 | TACCTTATGAGAAC | 1474 |
| | TD | UACT | | CAATGA | |
| hydroxy | AUS-H | UAUAUUUCCAGGAUGA | 1453 | ATATAAAGGTCCTA | 1475 |
| acid | | AAGU | | CTTTCA | |
| oxidase 1 | | | | | |

TABLE 8.2
Exemplary HBV Target Sequences

| HBV Target Sequence | SEQ ID NO: |
|---|---|
| CTTGG TCATG GGCCA TCAG | 1 |
| GCACT TCGCT TCACC TCTGC | 4 |
| TTTGTTTACGTCCCGTCGGC | 1484 |
| TTTACGTCCCGTCGGCGCTGAATCCTGCGG | 1485 |
| TTTACGTCCCGTCGGCGCTG | 1486 |
| TTTACGCGGACTCCCCGTCT | 1487 |
| TTGTTTACGTCCCGTCGGCG | 1488 |
| TTGTTGACAAGAATCCTCAC | 1489 |
| TTGAGAGAAGTCCACCACG | 1490 |
| TTCTCCTGGCTCAGTTTACTAGTGCCATTT | 1491 |
| TTCTCCTGGCTCAGTTTACT | 1492 |
| TTACGTCCCGTCGGCGCTGA | 1493 |
| TGTTTACGTCCCGTCGGCGC | 1494 |
| TGTGCCTTCTCATCTGCCGG | 1495 |
| TGTGCACTTCGCTTCACCTC | 1496 |
| TGGTGGACTTCTCTCAATTT | 1497 |
| TGGCTCAGTTTACTAGTGCC | 1498 |
| TGGACTTCTCTCAATTTTCTAGGGG | 1499 |
| TGGACTTCTCTCAATTTTCT | 1500 |
| TGCCTTCTCATCTGCCGGAC | 1501 |
| TGCCGGACCGTGTGCACTTC | 1502 |
| TGCCGATCCATACTGCGGAA | 1503 |
| TGCACTTCGCTTCACCTCTG | 1504 |
| TGACAAGAATCCTCACAATA | 1505 |
| TCTTTACGCGGACTCCCCGT | 1506 |
| TCTTGTTGACAAGAATCCTC | 1507 |
| TCTGTGCCTTCTCATCTGCC | 1508 |
| TCTGCCGATCCATACTGCGG | 1509 |
| TCTCTTTACGCGGACTCCCC | 1510 |
| TCTCATCTGCCGGACCGTGT | 1511 |
| TCGGCGCTGAATCCTGCGGA | 1512 |
| TCCTGGCTCAGTTTACTAGT | 1513 |
| TCCTCACAATACCGCAGAGT | 1514 |
| TCCCGTCGGCGCTGAATCCT | 1515 |
| TCCATACTGCGGAACTCCTA | 1516 |
| TCATCTGCCGGACCGTGTGC | 1517 |
| TCATCTCGAACGTTCACAGTCA | 1518 |
| TCAGTTTACTAGTGCCATTT | 1519 |
| TATTGTGAGGATTCTTGTCA | 1520 |
| TAGGAGGCTGTAGGCATAAATTGGTCTGCG | 1521 |
| TACGTCCCGTCGGCGCTGAA | 1522 |
| TACGCGGACTCCCCGTCTGT | 1523 |
| GTTTACGTCCCGTCGGCGCT | 1524 |
| GTTGGTGCACTTCGCTTCAC | 1525 |
| GTTGACAAGAATCCTCACAA | 1526 |
| GTTCCCAGTATGGATCGGC | 1527 |
| GTGTGCACTTCGCTTCACCT | 1528 |
| GTGGTGGACTTCTCTCAATT | 1529 |
| GTGGACTTCTCTCAATTTTCT | 1530 |
| GTGGACTTCTCTCAATTTTC | 1531 |
| GTGCCTTCTCATCTGCCGGA | 1532 |
| GTGCACTTCGCTTCACCTCT | 1533 |
| GTGAAGCGAAGTGCACACGG | 1534 |
| GTCTGTGCCTTCTCATCTGC | 1535 |
| GTCGGCGCTGAATCCTGCGG | 1536 |
| GTCCCGTCGGCGCTGAATCC | 1537 |
| GTATTGTGAGGATTCTTGTC | 1538 |
| GGTGGACTTCTCTCAATTTT | 1539 |
| GGGCGCACCTCTCTTTACGC | 1540 |
| GGCTGTAGGCATAAATTGGT | 1541 |
| GGCGCTGAATCCTGCGGACG | 1542 |
| GGAGGAACCTTCAGGGAAGG | 1543 |
| GGACTCCCCGTCTGTGCCTT | 1544 |
| GGACCGTGTGCACTTCGCTT | 1545 |
| GCTGTAGGCATAAATTGGT | 1546 |
| GCTGAATCCTGCGGACGACC | 1547 |
| GCTCAGTTTACTAGTGCCAT | 1548 |
| GCGGACTCCCCGTCTGTGCC | 1549 |
| GCGCTGAATCCTGCGGACGA | 1550 |
| GCGCACCTCTCTTTACGCGG | 1551 |
| GCCTTCTCATCTGCCGGACC | 1552 |
| GCCGGACCGTGTGCACTTCG | 1553 |
| GCCGATCCATACTGCGGAAC | 1554 |
| GCCGATCCATACTGCGGAA | 1555 |
| GCACTTCGCTTCACCTCTGC | 1556 |
| GCACTAGTAAACTGAGCCAG | 1557 |
| GCACCTCTCTTTACGCGGAC | 1558 |
| GATCCATACTGCGGAACTCC | 1559 |
| GACTCCCCGTCTGTGCCTTC | 1560 |
| GACCGTGTGCACTTCGCTTC | 1561 |
| GACAAGAATCCTCACAATAC | 1562 |
| GAATCCTCACAATACCGCAG | 1563 |
| GAAAATTGAGAGAAGTCCAC | 1564 |
| CTTTGTTTACGTCCCGTCGG | 1565 |
| CTTGTTGACAAGAATCCTCACAATACCGCA | 1566 |
| CTTCTCATCTGCCGGACCGT | 1567 |
| CTGTGCCTTCTCATCTGCCG | 1568 |
| CTGGCTCAGTTTACTAGTGC | 1569 |
| CTGGCTCAGTTTACTAGTG | 1570 |
| CTGCCGGACCGTGTGCACTT | 1571 |
| CTGCCGATCCATACTGCGGA | 1572 |
| CTCTGCCGATCCATACTGCG | 1573 |
| CTCCTGGCTCAGTTTACTAG | 1574 |
| CTCCCCGTCTGTGCCTTCTC | 1575 |
| CTCACAATACCGCAGAGTCT | 1576 |
| CTATCAAGGTATGTTGCCCGTTTGTCCTCT | 1577 |
| CGTGTGCACTTCGCTTCACC | 1578 |
| CGTGGTGGACTTCTCTCAATTTTCTAGGGG | 1579 |
| CGTGGTGGACTTCTCTCAATTTTCT | 1580 |
| CGTGGTGGACTTCTCTCAAT | 1581 |
| CGTGGTGGACTTCTCTCAAT | 1582 |
| CGTCGGCGCTGAATCCTGCG | 1583 |
| CGTCCCGTCGGCGCTGAATC | 1584 |
| CGGGGCGCACCTCTCTTTAC | 1585 |
| CGGCGCTGAATCCTGCGGAC | 1586 |
| CGGACTCCCCGTCTGTGCCT | 1587 |
| CGGACCGTGTGCACTTCGCT | 1588 |
| CGCTGAATCCTGCGGACGAC | 1589 |
| CGCGGACTCCCCGTCTGTGC | 1590 |
| CGCACCTCTCTTTACGCGGA | 1591 |
| CCTTTGTTTACGTCCCGTCG | 1592 |
| CCTTCTCATCTGCCGGACCG | 1593 |
| CCTTAGGCACTCCTGCCTCT | 1594 |
| CCTGGCTCAGTTTACTAGTG | 1595 |
| CCTCTGCCGATCCATACTGCGGAACTCCTA | 1596 |
| CCTCTGCCGATCCATACTGC | 1597 |
| CCTCTCTTTACGCGGACTCC | 1598 |
| CCTCACAATACCGCAGAGTC | 1599 |
| CCGTGTGCACTTCGCTTCAC | 1600 |
| CCGTGTGCACTTCGCTGTTG | 1601 |
| CCGTGTGCACTTCGCT | 1602 |
| CCGTCTGTGCCTTCTCATCT | 1603 |
| CCGTCGGCGCTGAATCCTGC | 1604 |
| CCGGTCCGTGTGCACTTCGC | 1605 |
| CCGGACCGTGTGCACTTCGC | 1606 |
| CCGATCCATACTGCGGAACT | 1607 |
| CCCGTCGGCGCTGAATCCTG | 1608 |
| CCCCGTCTGTGCCTTCTCAT | 1609 |
| CAGGTCCCCTAGAAGAAGAA | 1610 |
| CACGGGGCGCACCTCTCTTT | 1611 |
| CACCTCTCTTTACGCGGACT | 1612 |
| CAAGAATCCTCACAATACCG | 1613 |
| ATCTGCCGGACCGTGTGCAC | 1614 |
| ATCCTCACAATACCGCAGAG | 1615 |
| AGACTCGTGGTGGACTTCTCTCAATTTTCT | 1616 |
| AGAATCCTCACAATACCGCA | 1617 |
| ACGTCCCGTCGGCGCTGAAT | 1618 |
| ACGCGGACTCCCCGTCTGTG | 1619 |
| ACCTCTCTTTACGCGGACTC | 1620 |
| ACCGTGTGCACTTCGCTTCA | 1621 |
| ACCAATTTATGCCTACAGCG | 1622 |
| ACAAGAATCCTCACAATACC | 1623 |
| AATCCTCACAATACCGCAGA | 1624 |
| AAGGTATGTTGCCCGTTTGT | 1625 |
| AAGAATCCTCACAATACCGC | 1626 |
| CTAGACTCGTGGTGGACTTC | 1627 |
| CCTGCTGCTATGCCTCATCT | 1628 |
| CTGCTGCTATGCCTCATCTT | 1629 |
| TGCTGCTATGCCTCATCTTC | 1630 |
| GCTGCTATGCCTCATCTTCT | 1631 |
| CTGCTATGCCTCATCTTCTT | 1632 |
| CCTATGGGAGTGGGCCTCAG | 1633 |
| GCCATTTGTTCAGTGGTTCG | 1634 |
| GGAGGCTGTAGGCATAAATT | 1635 |
| GAGGCTGTAGGCATAAATTG | 1636 |
| AGGCTGTAGGCATAAATTGG | 1637 |
| GGCTGTAGGCATAAATTGGT | 1638 |
| TTCAAGCCTCCAAGCTGTGC | 1639 |
| TCAAGCCTCCAAGCTGTGCC | 1640 |
| CAAGCCTCCAAGCTGTGCCT | 1641 |
| AAGCCTCCAAGCTGTGCCTT | 1642 |
| AGCCTCCAAGCTGTGCCTTG | 1643 |
| GCCTCCAAGCTGTGCCTTGG | 1644 |
| GAACTCCCTCGCCTCGCAGA | 1645 |
| CACCATATTCTTGGGAACA | 1646 |

### HBV

Certain embodiments provide methods, modified multi-segmented antisense oligonucleotides, and compositions for inhibiting HBV mRNA expression.

Certain embodiments provide modified multi-segmented antisense oligonucleotides targeted to a HBV nucleic acid sequence. Exemplary HBV nucleic acid sequences include but are not limited to those shown in Table 8.3.

TABLE 8.3
Exemplary HBV sequences

| HBV | Accession | SEQ ID | |
|---|---|---|---|
| Genotype | No. | NO: | Sequence |
| GTD | U95551.1 | 3 | AATTCCACAACCTTTCACCAAACTCTGCAAGATCCCAGAGTGAGAGGCCT |
| (HepG | | | GTATTTCCCTGCTGGTGGCTCCAGTTCAGGAGCAGTAAACCCTGTTCCGA |
| 2.2.15 | | | CTACTGCCTCTCCCTTATCGTCAATCTTCTCGAGGATTGGGGACCCTGCG |
| cells) | | | CTGAACATGGAGAACATCACATCAGGATTCCTAGGACCCCTTCTCGTGTT |
| | | | ACAGGCGGGGTTTTTCTTGTTGACAAGAATCCTCACAATACCGCAGAGTC |
| | | | TAGACTCGTGGTGGACTTCTCTCAATTTTCTAGGGGGAACTACCGTGTGT |
| | | | CTTGGCCAAAATTCGCAGTCCCCAACCTCCAATCACTCACCAACCTCCTG |
| | | | TCCTCCAACTTGTCCTGGTTATCGCTGGATGTGTCTGCGGCGTTTTATCA |
| | | | TCTTCCTCTTCATCCTGCTGCTATGCCTCATCTTCTTGTTGGTTCTTCTG |
| | | | GACTATCAAGGTATGTTGCCCGTTTGTCCTCTAATTCCAGGATCCTCAAC |
| | | | CACCAGCACGGGACCATGCCGAACCTGCATGACTACTGCTCAAGGAACCT |
| | | | CTATGTATCCCTCCTGTTGCTGTACCAAACCTTCGGACGGAAATTGCACC |
| | | | TGTATTCCCATCCCATCATCCTGGGCTTTCGGAAAATTCCTATGGGAGTG |
| | | | GGCCTCAGCCCGTTTCTCCTGGCTCAGTTTACTAGTGCCATTTGTTCAGT |
| | | | GGTTCGTAGGGCTTTCCCCCACTGTTTGGCTTTCAGTTATATGGATGATG |
| | | | TGGTATTGGGGGCCAAGTCTGTACAGCATCTTGAGTCCCTTTTTACCGCT |
| | | | GTTACCAATTTTCTTTTGTCTTTGGGTATACATTTAAACCCTAACAAAAC |
| | | | AAAGAGATGGGGTTACTCTCTGAATTTTATGGGTTATGTCATTGGAAGTT |
| | | | ATGGGTCCTTGCCACAAGAACACATCATACAAAAAATCAAAGAATGTTTT |
| | | | AGAAAACTTCCTATTAACAGGCCTATTGATTGGAAAGTATGTCAACGAAT |
| | | | TGTGGGTCTTTTGGGTTTTGCTGCCCCATTTACACAATGTGGTTATCCTG |
| | | | CGTTAATGCCCTTGTATGCATGTATTCAATCTAAGCAGGCTTTCACTTTC |
| | | | TCGCCAACTTACAAGGCCTTTCTGTGTAAACAATACCTGAACCTTTACCC |
| | | | CGTTGCCCGGCAACGGCCAGGTCTGTGCCAAGTGTTTGCTGACGCAACCC |
| | | | CCACTGGCTGGGGCTTGGTCATGGGCCATCAGCGCGTGCGTGGAACCTTT |
| | | | TCGGCTCCTCTGCCGATCCATACTGCGGAACTCCTAGCCGCTTGTTTTGC |
| | | | TCGCAGCAGGTCTGGAGCAAACATTATCGGGACTGATAACTCTGTTGTCC |
| | | | TCTCCCGCAAATATACATCGTATCCATGGCTGCTAGGCTGTGCTGCCAAC |
| | | | TGGATCCTGCGCGGGACGTCCTTTGTTTACGTCCCGTCGGCGCTGAATCC |
| | | | TGCGGACGACCCTTCTCGGGGTCGCTTGGGACTCTCTCGTCCCCTTCTCC |
| | | | GTCTGCCGTTCCGACCGACCACGGGGCGCACCTCTCTTTACGCGGACTCC |
| | | | CCGTCTGTGCCTTCTCATCTGCCGGACCGTGTGCACTTCGCTTCACCTCT |
| | | | GCACGTCGCATGGAGACCACCGTGAACGCCCACCGAATGTTGCCCAAGGT |
| | | | CTTACATAAGAGGACTCTTGGACTCTCTGCAATGTCAACGACCGACCTTG |
| | | | AGGCATACTTCAAAGACTGTTTGTTTAAAGACTGGGAGGAGTTGGGGGAG |
| | | | GAGATTAGATTAAAGGTCTTTGTACTAGGAGGCTGTAGGCATAAATTGGT |
| | | | CTGCGCACCAGCACCATGCAACTTTTTCACCTCTGCCTAATCATCTCTTG |
| | | | TTCATGTCCTACTGTTCAAGCCTCCAAGCTGTGCCTTGGGTGGCTTTGGG |
| | | | GCATGGACATCGACCCTTATAAAGAATTTGGAGCTACTGTGGAGTTACTC |
| | | | TCGTTTTTGCCTTCTGACTTCTTTCCTTCAGTACGAGATCTTCTAGATAC |
| | | | CGCCTCAGCTCTGTATCGGGAAGCCTTAGAGTCTCCTGAGCATTGTTCAC |
| | | | CTCACCATACTGCACTCAGGCAAGCAATTCTTTGCTGGGGGGAACTAATG |
| | | | ACTCTAGCTACCTGGGTGGGTGTTAATTTGGAAGATCCAGCATCTAGAGA |
| | | | CCTAGTAGTCAGTTATGTCAACACTAATATGGGCCTAAAGTTCAGGCAAC |
| | | | TCTTGTGGTTTCACATTTCTTGTCTCACTTTTGGAAGAGAAACCGTTATA |
| | | | GAGTATTTGGTGTCTTTCGGAGTGTGGATTCGCACTCCTCCAGCTTATAG |
| | | | ACCACCAAATGCCCCTATCCTATCAACACTTCCGGAAACTACTGTTGTTA |
| | | | GACGACGAGGCAGGTCCCCTAGAAGAAGAACTCCCTCGCCTCGCAGACGA |
| | | | AGGTCTCAATCGCCGCGTCGCAGAAGATCTCAATCTCGGGAACCTCAATG |
| | | | TTAGTATTCCTTGGACTCATAAGGTGGGGAACTTTACTGGTCTTTATTCT |
| | | | TCTACTGTACCTGTCTTTAATCCTCATTGGAAAACACCATCTTTTCCTAA |
| | | | TATACATTTACACCAAGACATTATCAAAAAATGTGAACAGTTTGTAGGCC |
| | | | CACTTACAGTTAATGAGAAAAGAAGATTGCAATTGATTATGCCTGCTAGG |
| | | | TTTTATCCAAAGGTTACCAAATATTTACCATTGGATAAGGGTATTAAACC |
| | | | TTATTATCCAGAACATCTAGTTAATCATTACTTCCAAACTAGACACTATT |
| | | | TACACACTCTATGGAAGGCGGGTATATTATATAAGAGAGAAACAACACAT |
| | | | AGCGCCTCATTTTGTGGGTCACCATATTCTTGGGAACAAGATCTACAGCA |
| | | | TGGGGCAGAATCTTTCCACCAGCAATCCTCTGGGATTCTTTCCCGACCAC |
| | | | CAGTTGGATCCAGCCTTCAGAGCAAACACAGCAAATCCAGATTGGGACTT |
| | | | CAATCCCAACAAGGACACCTGGCCAGACGCCAACAAGGTAGGAGCTGGAG |
| | | | CATTCGGGCTGGGTTTCACCCCACCGCACGGAGGCCTTTTGGGGTGGAGC |
| | | | CCTCAGGCTCAGGGCATACTACAAACTTTGCCAGCAAATCCGCCTCCTGC |
| | | | CTCCACCAATCGCCAGACAGGAAGGCAGCCTACCCCGCTGTCTCCACCTT |
| | | | TGAGAAACACTCATCCTCAGGCCATGCAGTGG |
| GTA | AJ309369.1 | 1476 | TTCCACTGCCTTCCACCAAGCACTGCAGGATCCCAGAGTCAGGGGTCCGT |
| | | | ATTTTCCTGCTGGTGGCTCCAGTTCAGGAACAGTAAACCCTGCTCCGAAT |
| | | | ATTGCCTCTCACATCTCGTCAATCTCCGCGAGGACTGGGGACCCTGTGAC |
| | | | GAACATGGAGAACATCACATCAGGACTCCTAGGACCCCTGCTCGTGTTAC |
| | | | AGGCGGGGTTTTTCTTGTTGACAAGAATCCTCACAATACCGCAGAGTCTA |
| | | | GGCTCGTGGTGGACTTCTCTCAATTTTCTAGGGGGATCACCCGTGTGTCT |
| | | | TGGCCAAAATTCGCAGTCCCCAACCTCCAATCACTCACCAACCTCCTGTC |
| | | | CTCCAATTTGTCCTGGTTATCGTTGGATGTGTCTGCGGCGTTTTATCATA |
| | | | TTCCTCTTCATCCTGCTGCTATGCCTCATCTTCTTATTGGTTCTTCTGGA |
| | | | TTATCAAGGTATGTTGCCCGTTTGTCCTCTAATTCCAGGATCAACAACAA |
| | | | CCGGTACGGGACCATGCAAAACCTGCACGACTCCTGCTCAAGGCAACTCT |
| | | | ATGTTTCCCTCATGTTGCTGTACAAAACCTACGGACGGAAATTGCACCTG |
| | | | TATTCCCATCCCATCGTCCTGGGCTTTCGCAAAATACCTATGGGAGTGGG |
| | | | CCTCAGTCCGTTTCTCTTGGCTCAGTTTACTAGTGCCATTTGTTCAGTGG |
| | | | TTCGTAGGGCTTTCCCCCACTGTTTGGCTTTCAGCTATATGGATGATGTG |
| | | | GTATTGGGGGCCAAGACTGTACAGCATCGTGAGTCCCTTTATACCGCTGT |
| | | | TACCAATTTTCTTTTGTCTCTGGGTATACATTTAAACCCTAACAAAACAA |
| | | | AAAGATGGGGTTATTCCCTAAACTTCATGGGTTACATAATTGGAAGTTGG |
| | | | GGAACTTTGCCACAGGATCATATTGTACAAAAGATCAAACACTGTTTTAG |
| | | | AAAACTTCCTGTTAACAGGCCTATTGATTGGAAAGTATGTCAAAGAATTG |
| | | | TGGGTCTTTTGGGCTTTGCTGCTCCATTTACACAATGTGGATATCCTGCC |
| | | | TTAATGCCTTTGTATGCATGTATACAAGCTAAACAGGCTTTCACTTTCTC |
| | | | GCCAACTTACAAGGCCTTTCTAAGTAAACAGTACATGAACCTTTACCCCG |
| | | | TTGCTCGGCAACGGCCTGGTCTGTGCCAAGTGTTTGCTGACGCAACCCCC |
| | | | ACTGGCTGGGGCTTGGCCATAGGCCATCAGCGCATGCGTGGAACCTTTGT |
| | | | GGCTCCTCTGCCGATCCATACTGCGGAACTCCTAGCCGCTTGTTTTGCTC |
| | | | GCAGCCGGTCTGGAGCAAAGCTCATCGGAACTGACAATTCTGTCGTCCTC |
| | | | TCGCGGAAATATACATCGTTTCCATGGCTGCTAGGCTGTACTGCCAACTG |
| | | | GATCCTTCGCGGGACGTCCTTTGTTTACGTCCCGTCGGCGCTGAATCCCG |
| | | | CGGACGACCCCTCTCGGGGCCGCTTGGGACTCTATCGTCCCCTTCTCCGT |
| | | | CTGCCGTTCCAGCCGACCACGGGGCGCACCTCTCTTTACGCGGTCTCCCC |
| | | | GTCTGTGCCTTCTCATCTGCCGGTCCGTGTGCACTTCGCTTCACCTCTGC |
| | | | ACGTTGCATGGAGACCACCGTGAACGCCCATCAGATCCTGCCCAAGGTCT |
| | | | TACATAAGAGGACTCTTGGACTCCCAGCAATGTCAACGACCGACCTTGAG |
| | | | GCCTACTTCAAAGACTGTGTGTTTAAGGACTGGGAGGAGCTGGGGGAGGA |
| | | | GAGTAGGTTAATGATCTTTGTATTAGGAGGCTGTAGGCATAAATTGGTCT |
| | | | GCGCACCAGCACCATGCAACTTTTTCACCTCTGCCAAATCATCTCTTGTA |
| | | | CATGTCCCACTGTTCAAGCCTCCAAGCTGTGCCTTGGGTGGCTTTGGGGC |
| | | | ATGGACATTGACCCTTATAAAGAATTTGGAGCTACTGTTGAGTTACTCTC |
| | | | GTTTTTGCCTTCTGACTTCTTTCCTTCCGTCAGAGATCTTCTAGACACCG |
| | | | CCTCAGCTCTGTATCGAGAAGCCTTAGAGTCTCCTGAGCACTGCTCACCT |
| | | | CACCATACTGCACTCAGGCAAGCCATTCTCTGCTGGGGGGAATTGATGAC |
| | | | TCTAGCTACCTGGGTGGGTAATAATTTGGATGATCCAGCATCCAGGGATC |
| | | | TAGTAGTCAATTATGTTAATACTAACATGGGTTTAAAGATCAGGCAACTA |
| | | | TTGTGGTTTCATATATCTTGCCTTACTTTTGGAAGAGAGACTGTACTTGA |
| | | | ATATTTGGTCTCTTTCGGAGTGTGGATTCGCACTCCTCCACCCTATAGAC |
| | | | CACCAAATGCCCCTATCTTATCAACACTTCCGGAAACTACTGTTATTAGA |
| | | | CGACGGGACCGAGGCAGGTCCCCTAGAAGAAGAACTCCCTCGCCTCGCAG |
| | | | ACGCAGATCTCAATCGCCGCGTCGCAGAAGATCTCAATCTCGGGAATCTC |
| | | | AATGTTAGTATTCCTTGGACTCATAAGGTGGGAAACTTTACGGGGCTTTA |
| | | | TTCCTCTACAGTACCTATCTTTAATCCTGAATGGCAAACTCCTTCCTTTC |
| | | | CTAAGATTCATTTACAAGAGGACATTATTAATAGGTGTCAACAATTTGTG |
| | | | GGCCCTCTCACTGTAAATGAAAAGAGAAGATTGAAATTAATTATGCCTGC |
| | | | TAGATTCTATCCTACCCACACTAAATATTTGCCCTTAGACAAAGGAATTA |
| | | | AACCTTATTATCCAGATCAGGTAGTTAATCATTACTTCCAAACCAGACAT |
| | | | TATTTACATACTCTTTGGAAGGCGGGTATTCTATATAAGAGGGAAACCAC |
| | | | ACGTAGCGCATCATTTTGCGGGTCACCATATTCTTGGGAACAAGAGCTAC |
| | | | AGCATGGGAGGTTGGTCATCAAAACCTCGCAAAGGCATGGGGACGAATCT |
| | | | TTCTGTTCCCAACCCTCTGGGATTCTTTCCCGATCATCAGTTGGACCCTG |
| | | | CATTCGGAGCCAACTCAAACAATCCAGATTGGGACTTCAACCCCATCAAG |
| | | | GACCACTGGCCAGCAGCCAACCAGGTGGGAGTGGGGGCATTCGGGCCAGG |
| | | | GCTCACCCCTCCACACGGCGGTATCTTGGGGTGGAGCCCTCAGGCTCAGG |
| | | | GCATATTGACCACAGTGTCAACAATTCCTCCTCCTGCCTCCACCAATCGG |
| | | | CAGTCAGGAAGGCAGCCTACTCCCATCTCTCCACCTCTCCGAGACAGTCA |
| | | | TCCTCAGGCCACGCAGTGGAA |
| GTB | D00330.1 | 1477 | CTCCACCACTTTCCACCAAACTCTTCAAGATCCCGGAGTCAGGGCCCTGT |
| | | | ACTTTCCTGCTGGTGGCTCCAGTTCAGGAACAGTGAGCCCTGCTCAGAAT |
| | | | ACTGTCTCTGCCATATCGTCAATCTTATCGAAGACTGGGGACCCTGTACC |
| | | | GAACATGGAGAACATCGCATCAGGACTCCTAGGACCCCTGCTCGTGTTAC |
| | | | AGGCGGGGTTTTTCTTGTTGACAAAAATCCTCACAATACCACAGAGTCTA |
| | | | GACTCGTGGTGGACTTCTCTCAATTTTCTAGGGGGAACACCCGTGTGTCT |
| | | | TGGCCAAAATTCGCAGTCCCAAATCTCCAGTCACTCACCAACCTGTTGTC |
| | | | CTCCAATTTGTCCTGGTTATCGCTGGATGTGTCTGCGGCGTTTTATCATC |
| | | | TTCCTCTGCATCCTGCTGCTATGCCTCATCTTCTTGTTGGTTCTTCTGGA |
| | | | CTATCAAGGTATGTTGCCCGTTTGTCCTCTAATTCCAGGATCATCAACAA |
| | | | CCAGCACCGGACCATGCAAAACCTGCACAACTCCTGCTCAAGGAACCTCT |
| | | | ATGTTTCCCTCATGTTGCTGTACAAAACCTACGGATGGAAACTGCACCTG |
| | | | TATTCCCATCCCATCATCTTGGGCTTTCGCAAAATACCTATGGGAGTGGG |
| | | | CCTCAGTCCGTTTCTCTTGGCTCAGTTTACTAGTGCCATTTGTTCAGTGG |
| | | | TTCGTAGGGCTTTCCCCCACTGTCTGGCTTTCAGTTATATGGATGATATG |
| | | | GTTTTGGGGGCCAAGTCTGTACAACATCTTGAGTCCCTTTATGCCGCTGT |
| | | | TACCAATTTTCTTTTGTCTTTGGGTATACATTTAAACCCTCACAAAACAA |
| | | | AAAGATGGGGATATTCCCTTAACTTCATGGGATATGTAATTGGGAGCTGG |
| | | | GGCACATTGCCACAGGAACATATTGTACAAAAAATCAAAATGTGGTTTAG |
| | | | GAAACTTCCTGTAAACAGGCCTATTGATTGGAAAGTATGTCAACGAATTG |
| | | | TGGGTCTTTTGGGGTTTGCCGCCCCTTTCACGCAATGTGGATATCCTGCT |
| | | | TTAATGCCTTTATATGCATGTATACAAGCAAAACAGGCTTTTACTTTCTC |
| | | | GCCAACTTACAAGGCCTTTCTAACTAAACAGTATCTGAACCTTTACCCCG |
| | | | TTGCTCGGCAACGGCCAGGTCTGTGCCAAGTGTTTGCTGACGCAACCCCC |
| | | | ACTGGTTGGGGCTTGGCCATAGGCCATCAGCGCATGCGTGGAACCTTTGT |
| | | | GTCTCCTCTGCCGATCCATACTGCGGAACTCCTAGCCGCTTGTTTTGCTC |
| | | | GCAGCCGGTCTGGGGCAAAACTCATCGGGACTGACAATTCTGTCGTGCTC |
| | | | TCCCGCAAGTATACATCATTTCCATGGCTGCTAGGCTGTGCTGCCAACTG |
| | | | GATCCTGCGCGGGACGTCCTTTGTTTACGTCCCGTCGGCGCTGAATCCCG |
| | | | CGGACGACCCTTCCCGGGGCCGCTTGGGGCTCTACCGCCCGCTTCTCCGC |
| | | | CTGTTGTACCGACCGACCACGGGGCGCACCTCTCTTTACGCGGACTCCCC |
| | | | GTCTGTGCCTTCTCATCTGCCGGACCGTGTGCACTTCGCTTCACCTCTGC |
| | | | ACGTCGCATGGAGACCACCGTGAACGCCCACGGGAACCTGCCCAAGGTCT |
| | | | TGCATAAGAGGACTCTTGGACTTTCAGCAATGTCAACGACCGACCTTGAG |
| | | | GCATACTTCAAAGACTGTGTGTTTAATGAGTGGGAGGAGTTGGGGGAGGA |
| | | | GGTTAGGTTAAAGGTCTTTGTACTAGGAGGCTGTAGGCATAAATTGGTGT |
| | | | GTTCACCAGCACCATGCAACTTTTTCACCTCTGCCTAATCATCTCATGTT |
| | | | CATGTCCTACTGTTCAAGCCTCCAAGCTGTGCCTTGGGTGGCTTTGGGGC |
| | | | ATGGACATTGACCCGTATAAAGAATTTGGAGCTTCTGTGGAGTTACTCTC |
| | | | TTTTTTGCCTTCTGACTTCTTTCCGTCGGTGCGAGATCTCCTCGACACCG |
| | | | CCTCTGCTTTGTATCGGGAGGCCTTAGAGTCTCCGGAACATTGTTCACCT |
| | | | CACCATACGGCACTCAGGCAAGCTATTCTGTGTTGGGGTGAGTTAATGAA |
| | | | TCTAGCCACCTGGGTGGGAAGTAATTTGGAAGATCCGGCATCCAGGGAAT |
| | | | TAGTAGTCAGCTATGTCAACGTTAATATGGGCCTAAAAATCAGACAACTA |
| | | | TTGTGGTTTCACATTTCCTGTCTTACTTTTGGGAGAGAAACTGTTCTTGA |
| | | | ATATTTGGTGTCTTTTGGAGTGTGGATTCGCACTCCTCCTGCATATAGAC |
| | | | CACCAAATGCCCCTATCTTATCAACACTTCCGGAAACTACTGTTGTTAGA |
| | | | CGAAGAGGCAGGTCCCCTAGAAGAAGAACTCCCTCGCCTCGCAGACGAAG |
| | | | GTCTCAATCGCCGCGTCGCAGAAGATCTCAATCTCGGGAATCTCAATGTT |
| | | | AGTATTCCTTGGACACATAAGGTGGGAAACTTTACGGGGCTTTATTCTTC |
| | | | TACGGTACCTTGCTTTAATCCTAAATGGCAAACTCCTTCTTTTCCTGACA |
| | | | TTCATTTGCAGGAGGACATTGTTGATAGATGTAAGCAATTTGTGGGGCCC |
| | | | CTTACAGTAAATGAAAACAGGAGACTAAAATTAATTATGCCCGCTAGGTT |
| | | | TTATCCCAATGTTACTAAATATTTGCCCTTAGATAAAGGGATCAAACCGT |
| | | | ATTATCCAGAGTATGTAGTTAATCATTACTTCCAGACGCGACATTATTTA |
| | | | CACACTCTTTGGAAGGCGGGGATCTTATATAAAAGAGAGTCCACACGTAG |
| | | | CGCCTCATTTTGCGGGTCACCATATTCTTGGGAACAAGATCTACAGCATG |
| | | | GGAGGTTGGTCTTCCAAACCTCGAAAAGGCATGGGGACAAATCTTTCTGT |
| | | | CCCCAATCCTCTGGGATTCTTCCCCGATCATCAGTTGGACCCTGCATTCA |
| | | | AAGCCAACTCAGAAAATCCAGATTGGGACCTCAACCCGAACAAGGACAAC |
| | | | TGGCCGGACGCCAACAAGGTGGGAGTGGGAGCATTCGGGCCAGGGTTCAC |
| | | | CCCTCCCCATGGGGGACTGTTGGGGTGGAGCCCTCAGGCTCAGGGCCTAC |
| | | | TCACAACTGTGCCAGCAGCTCCTCCTCCTGCCTCCACCAATCGGCAGTCA |
| | | | GGAAGGCAGCCTACTCCCTTATCCCCACCTCTAAGGGACACTCATCCTCA |
| | | | GGCCATGCAGTGGAA |
| GTC | AB033550.1 | 1478 | CTCCACCACATTCCACCAAGCTCTGCTAGATCCCAGAGTGAGGGGCCTAT |
| | | | ATTTTCCTGCTGGTGGCTCCAGTTCCGGAACAGTAAACCCTGTTCCGACT |
| | | | ACTGCCTCACCCATATCGTCAATCTTCTCGAGGACTGGGGACCCTGCACA |
| | | | GAACATGGAGAACACAACATCAGGATTCCTAGGACCCCTGCTCGTGTTAC |
| | | | AGGCGGGGTTTTTCTTGTTGACAAAAATCCTCACAATACCACAGAGTCTA |
| | | | GACTCGTGGTGGACTTCTCTCAATTTTCTAGGGGGAGCACCCACGTGTCC |
| | | | TGGCCAAAATTCGCAGTCCCCAACCTCCAATCACTCACCAACCTCTTGTC |
| | | | CTCCAATTTGTCCTGGCTATCGCTGGATGTGTCTGCGGCGTTTTATCATA |
| | | | TTCCTCTTCATCCTGCTGCTATGCCTCATCTTCTTGTTGGTTCTTCTGGA |
| | | | CTACCAAGGTATGTTGCCCGTTTGTCCTCTACTTCCAGGAACAACAACTA |
| | | | CCAGCACGGGACCATGCAAGACCTGCACGATTCCTGCTCAAGGAACCTCT |
| | | | ATGTTTCCCTCTTGTTGCTGTACAAAACCTTCGGACGGAAACTGCACTTG |
| | | | TATTCCCATCCCATCATCCTGGGCTTTCGCAAGATTCCTATGGGAGTGGG |
| | | | CCTCAGTCCGTTTCTCCTGGCTCAGTTTACTAGTGCCATTTGTTCAGTGG |
| | | | TTCGTAGGGCTTTCCCCCACTGTTTGGCTTTCAGTTATATGGATGATGTG |
| | | | GTATTGGGGGCCAAGTCTGTACAACATCTTGAGTCCCTTTTTACCTCTAT |
| | | | TACCAATTTTCTTTTGTCTTTGGGTATACATTTGAATCCTAATAAAACCA |
| | | | AACGTTGGGGCTACTCCCTTAACTTCATGGGATATGTAATTGGAAGTTGG |
| | | | GGTACTTTACCACAGGAACATATTGTACGGAAACTCAAGCAATGTTTTCG |
| | | | AAAACTGCCTGTAAATAGACCTATTGATTGGAAAGTATGTCAAAGAATTG |
| | | | TGGGTCTTTTGGGCTTTGCTGCCCCTTTTACACAATGTGGCTATCCTGCC |
| | | | TTGATGCCTTTATATGCATGTATACACTCTAAGCAGGCTTTCACTTTCTC |
| | | | GCCAACTTACAAGGCCTTTCTGTGTAAACAATATCTGCACCTTTACCCCG |
| | | | TTGCCCGGCAACGGTCAGGTCTCTGCCAAGTGTTTGCTGACGCAACCCCC |
| | | | ACTGGATGGGGCTTGGCCATAGGCCATCGGCGCATGCGCGGAACCTTTGT |
| | | | GGCTCCTCTGCCGATCCATACTGCGGAACTCCTAGCAGCTTGTTTTGCTC |
| | | | GCAGCCGGTCTGGAGCAAAACTTATCGGGACTGACAACTCTGTTGTCCTC |
| | | | TCTCGGAAATACACCTCCTTCCCATGGCTGCTCGGGTGTGCTGCCAACTG |
| | | | GATCCTTCGCGGGACGTCCTTTGTCTACGTCCCGTCGGCGCTGAATCCCG |
| | | | CGGACGACCCGTCTCGGGGCCGTTTGGGGCTCTATCGTCCCCTTCTTCAT |
| | | | CTGCCGTTCCGGCCGACCACGGGGCGCACCTCTCTTTACGCGGTCTCCCC |
| | | | GTCTGTGCCTTCTCATCTGCCGGACCGTGTGCACTTCGCTTCACCTCTGC |
| | | | ACGTCGCATGGAGACCACCGTGAACGCCCACCAGGTCTTGCCCAAGGTCT |
| | | | TACATAAGAGGACTCTTGGACTCTCATCAATGTCAACGACCGACCTTGAG |
| | | | GCATACTTCAAAGACTGTTTGTTTAAGGACTGGGAGGAGTTGGGGGAGGA |
| | | | GATTAGGTTAAAGGTCTTTGTACTAGGAGGCTGTAGGCATAAATTGGTCT |
| | | | GTTCACCAGCACCATGCAACTTTTTCACCTCTGCCTAATCATCTCATGTT |
| | | | CATGTCCTACTGTTCAAGCCTCCAAGCTGTGCCTTGGGTGGCTTTGGGGC |
| | | | ATGGACATTGACCCGTATAAAGAATTTGGAGCTTCTGTGGAGTTACTCTC |
| | | | TTTTTTGCCTTCTGACTTCTTTCCTTCTATTCGAGATCTCCTCGACACCG |
| | | | CCTCTGCTCTGTATCGGGAGGCCTTAGAGTCTCCGGAACATTGTTCACCT |
| | | | CACCATACAGCACTCAGGCAAGCTATTCTGTGTTGGGGTGAGTTGATGAA |
| | | | TCTGGCCACCTGGGTGGGAAGTAATTTGGAAGACCCAGCATCCAGGGAAT |
| | | | TAGTAGTCAGCTATGTCAATGTTAATATGGGCCTAAAAATCAGACAACTA |
| | | | TTGTGGTTTCACATTTCCTGTCTTACTTTTGGAAGAGAAACTGTTCTTGA |
| | | | GTATTTGGTATCTTTTGGAGTGTGGATTCGCACTCCTCCAGCTTACAGAC |
| | | | CACCAAATGCCCCTATCTTATCAACACTTCCGGAAACTACTGTTGTTAGA |
| | | | CGACGAGGCAGGTCCCCTAGAAGAAGAACTCCCTCGCCTCGCAGACGAAG |
| | | | GTCTCAATCGCCGCGTCGCAGAAGATCTCAATCTCGGGAATCTCAATGTT |
| | | | AGTATCCCTTGGACTCATAAGGTGGGAAACTTTACTGGGCTTTATTCTTC |
| | | | TACTGTACCTGTCTTTAATCCTGAGTGGCAAACTCCCTCCTTTCCTAACA |
| | | | TTCATTTACAGGAGGACATTATTAATAGATGTGAACAATATGTGGGCCCT |
| | | | CTTACAACTAATGAAAAAAGGAGATTAAAATTAATTATGCCTGCTAGGTT |
| | | | TTATCCTAACCTTACCAAATACTTGCCCTTGGATAAAGGCATTAAACCTT |
| | | | ATTATCCTGAACATGCAGTTAATCATTACTTCAAAACTAGGCATTATTTA |
| | | | CATACTCTGTGGAAGGCCGGCATTCTATATAAGAGAGAAACTACACGCAG |
| | | | CGCTTCATTTTGTGGGTCACCATATTCTTGGGAACAAGAGCTACAGCATG |
| | | | GGAGGTTGGTCTTCCAAACCTCGACAAGGCATGGGGACGAATCTTTCTGT |
| | | | TCCCAATCCTCTGGGATTTTTTCCCGATCACCAGTTGGACCCTGCGTTCG |
| | | | GAGCCAACTCAAACAATCCAGATTGGGACTTCAACCCCAACAAGGATCAC |
| | | | TGGCCAGAGGCAAATCAGGTAGGAGCGGGAGCATTCGGGCCAGGGTTCAC |
| | | | CCCACCACACGGCGGTCTTTTGGGGTGGAGCCCTCAGGCTCAGGGCATAT |
| | | | TGACCACAGTGCCAGCAGCGCCTCCTCCTGCCTCCACCAATCGGCAGTCA |
| | | | GGAAGACAGCCTACTCCCATCTCTCCACCTCTAAGAGACAGTCATCCTCA |
| | | | GGCCATGCAGTGGAA |
| GTD | KX470733.1 | 1479 | AAAAACCTCCCACACCTCCCCCTGAACCTGAAACATAAAATGAATGCAAT |
| | | | TGTTGTTGTTAACTTGTTTATTGCAGCTTATAATGGTTACAAATAAAGCA |
| | | | ATAGCATCACAAATTTCACAAATAAAGCATTTTTTTCACTGCATTCTAGT |
| | | | TGTGGTTTGTCCAAACTCATCAATGTATCTTATCATGTCTGGATCTGGGC |
| | | | CACTCCCTCTCTGCGCGCTCGCTCGCTCACTGAGGCCGCCCGGGCTTTGC |
| | | | CCGGGCGGCCTCAGTGAGCGAGCGAGCGCGCAGAGAGGGAGTGGCCAACT |
| | | | CCATCACTAGGGGTTCCTCTGCTCGATGTATTCAATCTAAGCAGGCTTTC |
| | | | ACTTTCTCGCCAACTTACAAGGCCTTTCTGTGTAAACAATACCTGAACCT |
| | | | TTACCCCGTTGCCCGGCAACGGCCAGGTCTGTGCCAAGTGTTTGCTGACG |
| | | | CAACCCCCACTGGCTGGGGCTTGGTCATGGGCCATCAGCGCGTGCGTGGA |
| | | | ACCTTTTCGGCTCCTCTGCCGATCCATACTGCGGAACTCCTAGCCGCTTG |
| | | | TTTTGCTCGCAGCAGGTCTGGAGCAAACATTATCGGGACTGATAACTCTG |
| | | | TTGTCCTCTCCCGCAAATATACATCGTATCCATGGCTGCTAGGCTGTGCT |
| | | | GCCAACTGGATCCTGCGCGGGACGTCCTTTGTTTACGTCCCGTCGGCGCT |
| | | | GAATCCTGCGGACGACCCTTCTCGGGGTCGCTTGGGACTCTCTCGTCCCC |
| | | | TTCTCCGTCTGCCGTTCCGACCGACCACGGGGCGCACCTCTCTTTACGCG |
| | | | GACTCCCCGTCTGTGCCTTCTCATCTGCCGGACCGTGTGCACTTCGCTTC |
| | | | ACCTCTGCACGTCGCATGGAGACCACCGTGAACGCCCACCGAATGTTGCC |
| | | | CAAGGTCTTACATAAGAGGACTCTTGGACTCTCTGCAATGTCAACGACCG |
| | | | ACCTTGAGGCATACTTCAAAGACTGTTTGTTTAAAGACTGGGAGGAGTTG |
| | | | GGGGAGGAGATTAGATTAAAGGTCTTTGTACTAGGAGGCTGTAGGCATAA |
| | | | ATTGGTCTGCGCACCAGCACCATGCAACTTTTTCACCTCTGCCTAATCAT |
| | | | CTCTTGTTCATGTCCTACTGTTCAAGCCTCCAAGCTGTGCCTTGGGTGGC |
| | | | TTTGGGGCATGGACATCGACCCTTATAAAGAATTTGGAGCTACTGTGGAG |
| | | | TTACTCTCGTTTTTGCCTTCTGACTTCTTTCCTTCAGTACGAGATCTTCT |
| | | | AGATACCGCCTCAGCTCTGTATCGGGAAGCCTTAGAGTCTCCTGAGCATT |
| | | | GTTCACCTCACCATACTGCACTCAGGCAAGCAATTCTTTGCTGGGGGGAA |
| | | | CTAATGACTCTAGCTACCTGGGTGGGTGTTAATTTGGAAGATCCAGCATC |
| | | | GGCAACTCTTGTGGTTTCACATTTCTTGTCTCACTTTTGGAAGAGAAACC |
| | | | GTTATAGAGTATTTGGTGTCTTTCGGAGTGTGGATTCGCACTCCTCCAGC |
| | | | TTATAGACCACCAAATGCCCCTATCCTATCAACACTTCCGGAAACTACTG |
| | | | TTGTTAGACGACGAGGCAGGTCCCCTAGAAGAAGAACTCCCTCGCCTCGC |
| | | | AGACGAAGGTCTCAATCGCCGCGTCGCAGAAGATCTCAATCTCGGGAACC |
| | | | TCAATGTTAGTATTCCTTGGACTCATAAGGTGGGGAACTTTACTGGTCTT |
| | | | TATTCTTCTACTGTACCTGTCTTTAATCCTCATTGGAAAACACCATCTTT |
| | | | TCCTAATATACATTTACACCAAGACATTATCAAAAAATGTGAACAGTTTG |
| | | | TAGGCCCACTTACAGTTAATGAGAAAAGAAGATTGCAATTGATTATGCCT |
| | | | GCTAGGTTTTATCCAAAGGTTACCAAATATTTACCATTGGATAAGGGTAT |
| | | | TAAACCTTATTATCCAGAACATCTAGTTAATCATTACTTCCAAACTAGAC |
| | | | ACTATTTACACACTCTATGGAAGGCGGGTATATTATATAAGAGAGAAACA |
| | | | ACACATAGCGCCTCATTTTGTGGGTCACCATATTCTTGGGAACAAGATCT |
| | | | ACAGCATGGGGCAGAATCTTTCCACCAGCAATCCTCTGGGATTCTTTCCC |
| | | | GACCACCAGTTGGATCCAGCCTTCAGAGCAAACACAGCAAATCCAGATTG |
| | | | GGACTTCAATCCCAACAAGGACACCTGGCCAGACGCCAACAAGGTAGGAG |
| | | | CTGGAGCATTCGGGCTGGGTTTCACCCCACCGCACGGAGGCCTTTTGGGG |
| | | | TGGAGCCCTCAGGCTCAGGGCATACTACAAACTTTGCCAGCAAATCCGCC |
| | | | TCCTGCCTCCACCAATCGCCAGACAGGAAGGCAGCCTACCCCGCTGTCTC |
| | | | CACCTTTGAGAAACACTCATCCTCAGGCCATGCAGTGGAATTCCACAACC |
| | | | TTTCACCAAACTCTGCAAGATCCCAGAGTGAGAGGCCTGTATTTCCCTGC |
| | | | TGGTGGCTCCAGTTCAGGAGCAGTAAACCCTGTTCCGACTACTGCCTCTC |
| | | | CCTTATCGTCAATCTTCTCGAGGATTGGGGACCCTGCGCTGAACATGGAG |
| | | | AACATCACATCAGGATTCCTAGGACCCCTTCTCGTGTTACAGGCGGGGTT |
| | | | TTTCTTGTTGACAAGAATCCTCACAATACCGCAGAGTCTAGACTCGTGGT |
| | | | GGACTTCTCTCAATTTTCTAGGGGGAACTACCGTGTGTCTTGGCCAAAAT |
| | | | TCGCAGTCCCCAACCTCCAATCACTCACCAACCTCCTGTCCTCCAACTTG |
| | | | TCCTGGTTATCGCTGGATGTGTCTGCGGCGTTTTATCATCTTCCTCTTCA |
| | | | TCCTGCTGCTATGCCTCATCTTCTTGTTGGTTCTTCTGGACTATCAAGGT |
| | | | ATGTTGCCCGTTTGTCCTCTAATTCCAGGATCCTCAACCACCAGCACGGG |
| | | | ACCATGCCGAACCTGCATGACTACTGCTCAAGGAACCTCTATGTATCCCT |
| | | | CCTGTTGCTGTACCAAACCTTCGGACGGAAATTGCACCTGTATTCCCATC |
| | | | CCATCATCCTGGGCTTTCGGAAAATTCCTATGGGAGTGGGCCTCAGCCCG |
| | | | TTTCTCCTGGCTCAGTTTACTAGTGCCATTTGTTCAGTGGTTCGTAGGGC |
| | | | TTTCCCCCACTGTTTGGCTTTCAGTTATATGGATGATGTGGTATTGGGGG |
| | | | CCAAGTCTGTACAGCATCTTGAGTCCCTTTTTACCGCTGTTACCAATTTT |
| | | | CTTTTGTCTTTGGGTATACATTTAAACCCTAACAAAACAAAGAGATGGGG |
| | | | TTACTCTCTGAATTTTATGGGTTATGTCATTGGAAGTTATGGGTCCTTGC |
| | | | CACAAGAACACATCATACAAAAAATCAAAGAATGTTTTAGAAAACTTCCT |
| | | | ATTAACAGGCCTATTGATTGGAAAGTATGTCAACGAATTGTGGGTCTTTT |
| | | | GGGTTTTGCTGCCCCATTTACACAATGTGGTTATCCTGCGTTAATGCCCT |
| | | | TGTATGCATGTATTCAATCTAAGCAGGCTTTCACTTTCTCGCCAACTTAC |
| | | | AAGGCCTTTCTGTGTAAACAATACCTGAACCTTTACCCCGTTGCCCGGCA |
| | | | ACGGCCAGGTCTGTGCCAAGTGTTTGCTGACGCAACCCCCACTGGCTGGG |
| | | | GCTTGGTCATGGGCCATCAGCGCGTGCGTGGAACCTTTTCGGCTCCTCTG |
| | | | CCGATCCATACTGCGGAACTCCTAGCCGCTTGTTTTGCTCGCAGCAGGTC |
| | | | TGGAGCAAACATTATCGGGACTGATAACTCTGTTGTCCTCTCCCGCAAAT |
| | | | ATACATCGTATCCATGGCTGCTAGGCTGTGCTGCCAACTGGATCCTGCGC |
| | | | GGGACGTCCTTTGTTTACGTCCCGTCGGCGCTGAATCCTGCGGACGACCC |
| | | | TTCTCGGGGTCGCTTGGGACTCTCTCGTCCCCTTCTCCGTCTGCCGTTCC |
| | | | GACCGACCACGGGGCGCACCTCTCTTTACGCGGACTCCCCGTCTGTGCCT |
| | | | TCTCATCTGCCGGACCGTGTGCACTTCGCTTCACCTCTGCACGTCGCATG |
| | | | GAGACCACCGTGAACGCCCACCGAATGTTGCCCAAGGTCTTACATAAGAG |
| | | | GACTCTTGGACTCTCTGCAATGTCAACGACCGACCTTGAGGCATACTTCA |
| | | | AAGACTGTTTGTTTAAAGACTGGGAGGAGTTGGGGGAGGAGATTAGATTA |
| | | | AAGGTCTTTGTACTAGGAGGCTGTAGGCATAAATTGGTCTGCGCACCAGC |
| | | | ACCATGCAACTTTTTCACCTCTGCCTAATCATCTCTTGTTCATGTCCTAC |
| | | | TGTTCAAGCCTCCAAGCTGTGCCTTGGGTGGCTTTGGGGCATGGACATCG |
| | | | ACCCTTATAAAGAATTTGGAGCTACTGTGGAGTTACTCTCGTTTTTGCCT |
| | | | TCTGACTTCTTTCCTTCAGTACGAGATCCCCTAACTACAAGGAACCCCTA |
| | | | GTGATGGAGTTGGCCACTCCCTCTCTGCGCGCTCGCTCGCTCACTGAGGC |
| | | | CGGGCGACCAAAGGTCGCCCGACGCCCGGGCTTTGCCCGGGCGGCCTCAG |
| | | | TGAGCGAGCGAGCGCGCAGAGAGGGACAGATCCCCAGGAAGCTCCTCTGT |
| | | | GTCCTCATAAACCCTAACCTCCTCTACTTGAGAGGACATTCCAATCATAG |
| | | | GCTGCCCATCCACCCTCTGTGTCCTCCTGTTAATTAGGTCACTTAACAAA |
| | | | AAGGAAATTGGGTAGGGGTTTTTCACAGACCGCTTTCTAAGGGTAATTTT |
| | | | AAAATATCTGGGAAGTCCCTTCCACTGCTGTGTTCCAGAAGTGTTGGTAA |
| | | | ACAGCCCACAAATGTCAACAGCAGAAACATACAAGCTGTCAGCTTTGCAC |
| | | | AAGGGCCCAACACCCTGCTCATCAAGAAGCACTGTGGTTGCTGTGTTAGT |
| | | | AATGTGCAAAACAGGAGGCACATTTTCCCCACCTGTGTAGGTTCCAAAAT |
| | | | ATCTAGTGTTTTCATTTTTACTTGGATCAGGAACCCAGCACTCCACTGGA |
| | | | TAAGCATTATCCTTATCCAAAACAGCCTTGTGGTCAGTGTTCATCTGCTG |
| | | | ACTGTCAACTGTAGCATTTTTTGGGGTTACAGTTTGAGCAGGATATTTGG |
| | | | TCCTGTAGTTTGCTAACACACCCTGCAGCTCCAAAGGTTCCCCACCAACA |
| | | | GCAAAAAAATGAAAATTTGACCCTTGAATGGGTTTTCCAGCACCATTTTC |
| | | | ATGAGTTTTTTGTGTCCCTGAATGCAAGTTTAACATAGCAGTTACCCCAA |
| | | | TAACCTCAGTTTTAACAGTAACAGCTTCCCACATCAAAATATTTCCACAG |
| | | | GTTAAGTCCTCATTTAAATTAGGCAAAGGAATTAATTCTTGAAGACGAAA |
| | | | GGGCCTCGTGATACGCCTATTTTTATAGGTTAATGTCATGATAATAATGG |
| | | | TTTCTTAGACGTCAGGTGGCACTTTTCGGGGAAATGTGCGCGGAACCCCT |
| | | | ATTTGTTTATTTTTCTAAATACATTCAAATATGTATCCGCTCATGAGACA |
| | | | ATAACCCTGATAAATGCTTCAATAATATTGAAAAAGGAAGAGTATGAGTA |
| | | | TTCAACATTTCCGTGTCGCCCTTATTCCCTTTTTTGCGGCATTTTGCCTT |
| | | | CCTGTTTTTGCTCACCCAGAAACGCTGGTGAAAGTAAAAGATGCTGAAGA |
| | | | TCAGTTGGGTGCACGAGTGGGTTACATCGAACTGGATCTCAACAGCGGTA |
| | | | AGATCCTTGAGAGTTTTCGCCCCGAAGAACGTTTTCCAATGATGAGCACT |
| | | | TTTAAAGTTCTGCTATGTGGCGCGGTATTATCCCGTGTTGACGCCGGGCA |
| | | | AGAGCAACTCGGTCGCCGCATACACTATTCTCAGAATGACTTGGTTGAGT |
| | | | ACTCACCAGTCACAGAAAAGCATCTTACGGATGGCATGACAGTAAGAGAA |
| | | | TTATGCAGTGCTGCCATAACCATGAGTGATAACACTGCGGCCAACTTACT |
| | | | TCTGACAACGATCGGAGGACCGAAGGAGCTAACCGCTTTTTTGCACAACA |
| | | | TGGGGGATCATGTAACTCGCCTTGATCGTTGGGAACCGGAGCTGAATGAA |
| | | | GCCATACCAAACGACGAGCGTGACACCACGATGCCTGCAGCAATGGCAAC |
| | | | AACGTTGCGCAAACTATTAACTGGCGAACTACTTACTCTAGCTTCCCGGC |
| | | | AACAATTAATAGACTGGATGGAGGCGGATAAAGTTGCAGGACCACTTCTG |
| | | | CGCTCGGCCCTTCCGGCTGGCTGGTTTATTGCTGATAAATCTGGAGCCGG |
| | | | TGAGCGTGGGTCTCGCGGTATCATTGCAGCACTGGGGCCAGATGGTAAGC |
| | | | CCTCCCGTATCGTAGTTATCTACACGACGGGGAGTCAGGCAACTATGGAT |
| | | | GAACGAAATAGACAGATCGCTGAGATAGGTGCCTCACTGATTAAGCATTG |
| | | | GTAACTGTCAGACCAAGTTTACTCATATATACTTTAGATTGATTTAAAAC |
| | | | TTCATTTTTAATTTAAAAGGATCTAGGTGAAGATCCTTTTTGATAATCTC |
| | | | ATGACCAAAATCCCTTAACGTGAGTTTTCGTTCCACTGAGCGTCAGACCC |
| | | | CGTAGAAAAGATCAAAGGATCTTCTTGAGATCCTTTTTTTCTGCGCGTAA |
| | | | TCTGCTGCTTGCAAACAAAAAAACCACCGCTACCAGCGGTGGTTIGTTTG |
| | | | CCGGATCAAGAGCTACCAACTCTTTTTCCGAAGGTAACTGGCTTCAGCAG |
| | | | AGCGCAGATACCAAATACTGTCCTTCTAGTGTAGCCGTAGTTAGGCCACC |
| | | | ACTTCAAGAACTCTGTAGCACCGCCTACATACCTCGCTCTGCTAATCCTG |
| | | | TTACCAGTGGCTGCTGCCAGTGGCGATAAGTCGTGTCTTACCGGGTTGGA |
| | | | CTCAAGACGATAGTTACCGGATAAGGCGCAGCGGTCGGGCTGAACGGGGG |
| | | | GTTCGTGCACACAGCCCAGCTTGGAGCGAACGACCTACACCGAACTGAGA |
| | | | TACCTACAGCGTGAGCTATGAGAAAGCGCCACGCTTCCCGAAGGGAGAAA |
| | | | GGCGGACAGGTATCCGGTAAGCGGCAGGGTCGGAACAGGAGAGCGCACGA |
| | | | GGGAGCTTCCAGGGGGAAACGCCTGGTATCTTTATAGTCCTGTCGGGTTT |
| | | | CGCCACCTCTGACTTGAGCGTCGATTTTTGTGATGCTCGTCAGGGGGGCG |
| | | | GAGCCTATGGAAAAACGCCAGCAACGCGGCCTTTTTACGGTTCCTGGCCT |
| | | | TTTGCTGGCCTTTTGCTCACATGTTCTTTCCTGCGTTATCCCCTGATTCT |
| | | | GTGGATAACCGTATTACCGCCTTTGAGTGAGCTGATACCGCTCGCCGCAG |
| | | | CCGAACGACCGAGCGCAGCGAGTCAGTGAGCGAGGAAGCGGAAGAGCGCC |
| | | | TGATGCGGTATTTTCTCCTTACGCATCTGTGCGGTATTTCACACCGCATA |
| | | | TGGTGCACTCTCAGTACAATCTGCTCTGATGCCGCATAGTTAAGCCAGTA |
| | | | TACACTCCGCTATCGCTACGTGACTGGGTCATGGCTGCGCCCCGACACCC |
| | | | GCCAACACCCGCTGACGCGCCCTGACGGGCTTGTCTGCTCCCGGCATCCG |
| | | | CTTACAGACAAGCTGTGACCGTCTCCGGGAGCTGCATGTGTCAGAGGTTT |
| | | | TCACCGTCATCACCGAAACGCGCGAGGCAGCTGTGGAATGTGTGTCAGTT |
| | | | AGGGTGTGGAAAGTCCCCAGGCTCCCCAGCAGGCAGAAGTATGCAAAGCA |
| | | | TGCATCTCAATTAGTCAGCAACCAGGTGTGGAAAGTCCCCAGGCTCCCCA |
| | | | GCAGGCAGAAGTATGCAAAGCATGCATCTCAATTAGTCAGCAACCATAGT |
| | | | CCCGCCCCTAACTCCGCCCATCCCGCCCCTAACTCCGCCCAGTTCCGCCC |
| | | | ATTCTCCGCCCCATGGCTGACTAATTTTTTTTATTTATGCAGAGGCCGAG |
| | | | GCCGCCTCGGCCTCTGAGCTATTCCAGAAGTAGTGAGGAGGCTTTTTTGG |
| | | | AGGCCTAGGCTTTTGCAAAAAGCTTCACGCTGCCGCAAGCACTCAGGGCG |
| | | | CAAGGGCTGCTAAAGGAAGCGGAACACGTAGAAAGCCAGTCCGCAGAAAC |
| | | | GGTGCTGACCCCGGATGAATGTCAGCTACTGGGCTATCTGGACAAGGGAA |
| | | | AACGCAAGCGCAAAGAGAAAGCAGGTAGCTTGCAGTGGGCTTACATGGCG |
| | | | ATAGCTAGACTGGGCGGTTTTATGGACAGCAAGCGAACCGGAATTGCCAG |
| | | | CTGGGGCGCCCTCTGGTAAGGTTGGGAAGCCCTGCAAAGTAAACTGGATG |
| | | | GCTTTCTTGCCGCCAAGGATCTGATGGCGCAGGGGATCAAGATCGATCTG |
| | | | ATCAAGAGACAGGATGAGGATCGTTTCGCATGATTGAACAAGATGGATTG |
| | | | CACGCAGGTTCTCCGGCCGCTTGGGTGGAGAGGCTATTCGGCTATGACTG |
| | | | GGCACAACAGACAATCGGCTGCTCTGATGCCGCCGTGTTCCGGCTGTCAG |
| | | | CGCAGGGGCGCCCGGTTCTTTTTGTCAAGACCGACCTGTCCGGTGCCCTG |
| | | | AATGAACTGCAGGACGAGGCAGCGCGGCTATCGTGGCTGGCCACGACGGG |
| | | | CGTTCCTTGCGCAGCTGTGCTCGACGTTGTCACTGAAGCGGGAAGGGACT |
| | | | GGCTGCTATTGGGCGAAGTGCCGGGGCAGGATCTCCTGTCATCTCACCTT |
| | | | GCTCCTGCCGAGAAAGTATCCATCATGGCTGATGCAATGCGGCGGCTGCA |
| | | | TACGCTTGATCCGGCTACCTGCCCATTCGACCACCAAGCGAAACATCGCA |
| | | | TCGAGCGAGCACGTACTCGGATGGAAGCCGGTCTTGTCGATCAGGATGAT |
| | | | CTGGACGAAGAGCATCAGGGGCTCGCGCCAGCCGAACTGTTCGCCAGGCT |
| | | | CAAGGCGCGCATGCCCGACGGCGAGGATCTCGTCGTGACCCATGGCGATG |
| | | | CCTGCTTGCCGAATATCATGGTGGAAAATGGCCGCTTTTCTGGATTCATC |
| | | | GACTGTGGCCGGCTGGGTGTGGCGGACCGCTATCAGGACATAGCGTTGGC |
| | | | TACCCGTGATATTGCTGAAGAGCTTGGCGGCGAATGGGCTGACCGCTTCC |
| | | | TCGTGCTTTACGGTATCGCCGCTCCCGATTCGCAGCGCATCGCCTTCTAT |
| | | | CGCCTTCTTGACGAGTTCTTCTGAGCGGGACTCTGGGGTTCGAAATGACC |
| | | | GACCAAGCGACGCCCAACCTGCCATCACGAGATTTCGATTCCACCGCCGC |
| | | | CTTCTATGAAAGGTTGGGCTTCGGAATCGTTTTCCGGGACGCCGGCTGGA |
| | | | TGATCCTCCAGCGCGGGGATCTCATGCTGGAGTTCTTCGCCCACCCCGGG |
| | | | CTCGATCCCCTCGCGAGTTGGTTCAGCTGCTGCCTGAGGCTGGACGACCT |
| | | | CGCGGAGTTCTACCGGCAGTGCAAATCCGTCGGCATCCAGGAAACCAGCA |
| | | | GCGGCTATCCGCGCATCCATGCCCCCGAACTGCAGGAGTGGGGAGGCACG |
| | | | ATGGCCGCTTTGGTCCGGATCTTTGTGAAGGAACCTTACTTCTGTGGTGT |
| | | | GACATAATTGGACAAACTACCTACAGAGATTTAAAGCTCTAAGGTAAATA |
| | | | TAAAATTTTTAAGTGTATAATGTGTTAAACTACTGATTCTAATTGTTTGT |
| | | | GTATTTTAGATTCCAACCTATGGAACTGATGAATGGGAGCAGTGGTGGAA |
| | | | TGCCTTTAATGAGGAAAACCTGTTTTGCTCAGAAGAAATGCCATCTAGTG |
| | | | ATGATGAGGCTACTGCTGACTCTCAACATTCTACTCCTCCAAAAAAGAAG |
| | | | AGAAAGGTAGAAGACCCCAAGGACTTTCCTTCAGAATTGCTAAGTTTTTT |
| | | | GAGTCATGCTGTGTTTAGTAATAGAACTCTTGCTTGCTTTGCTATTTACA |
| | | | CCACAAAGGAAAAAGCTGCACTGCTATACAAGAAAATTATGGAAAAATAT |
| | | | TCTGTAACCTTTATAAGTAGGCATAACAGTTATAATCATAACATACTGTT |
| | | | TTTTCTTACTCCACACAGGCATAGAGTGTCTGCTATTAATAACTATGCTC |
| | | | AAAAATTGTGTACCTTTAGCTTTTTAATTTGTAAAGGGGTTAATAAGGAA |
| | | | TATTTGATGTATAGTGCCTTGACTAGAGATCATAATCAGCCATACCACAT |
| | | | TTGTAGAGGTTTTACTTGCTTTA |
| GTE | KX186584 | 1480 | TTCCACAACATTCCACCAAGCTCTGCAGGATCCCAGAGTAAGAGGCCTGT |
| | | | ATTTTCCTGCTGGTGGCTCCAGTTCCGGAACAGTGAACCCTGTTCCGACT |
| | | | ACTGCCTCACTCATCTCGTCAATCTTCTCGAGGATTGGGGACCCTGCACC |
| | | | GAACATGGAAAGCATCACATCAGGATTCCTAGGACCCCTGCTCGTGTTAC |
| | | | AGGCGGGGTTTTTCTTGTTGACAAAAATCCTCACAATACCGCAGAGTCTA |
| | | | GACTCGTGGTGGACTTCTCTCAATTTTCTAGGGGGAGCTCCCGTGTGTCT |
| | | | TGGCCAAAATTCGCAGTCCCCAACCTCCAATCACTCACCAACCTCTTGTC |
| | | | CTCCAATTTGTCCTGGCTATCGCTGGATGTGTCTGCGGCGTTTTATCATC |
| | | | TTCCTCTTCATCCTGCTGCTATGCCTCATCTTCTTGTTGGTTCTTCTGGA |
| | | | CTATCAAGGTATGTTGCCCGTTTGTCCTCTAATTCCAGGATCATCAACCA |
| | | | CCAGTACGGGACCCTGCCGAACCTGCACGACTCTTGCTCAAGGAACCTCT |
| | | | ATGTTTCCCTCATGTTGCTGTTCAAAACCTTCGGACGGAAATTGCACTTG |
| | | | TATTCCCATCCCATCATCATGGGCTTTCGGAAAATTCCTATGGGAGTGGG |
| | | | CCTCAGCCCGTTTCTCCTGGCTCAGTTTACTAGTGCCATTTGTTCAGTGG |
| | | | TTCGCCGGGCTTTCCCCCACTGTCTGGCTTTCAGTTATATGGATGATGTG |
| | | | GTATTGGGGGCCAAGTCTGTACAACATCTTGAGTCCCTTTATACCTCTGT |
| | | | TACCAATTTTCTTTTGTCTTTGGGTATACATTTAAATCCCAACAAAACAA |
| | | | AAAGATGGGGATATTCCCTAAATTTCATGGGTTATGTAATTGGAAGTTGG |
| | | | GGGTCATTACCACAGGAACACATCAGAATGAAAATCAAAGACTGTTTTAG |
| | | | AAAACTCCCTGTTAACCGGCCTATTGATTGGAAAGTATGTCAAAGAATTG |
| | | | TGGGTCTTTTGGGCTTTGCTGCCCCTTTTACACAATGTGGATATCCTGCT |
| | | | TTAATGCCTCTGTATGCGTGTATTCAATCTAAGCAGGCTTTCACTTTCTC |
| | | | GCCAACTTACAAGGCCTTTCTGTGTAAACAATACCTGAACCTTTACCCCG |
| | | | TTGCCCGGCAACGGCCAGGTCTGTGCCAAGTGTTTGCTGATGCAACCCCC |
| | | | ACTGGCTGGGGCTTGGCCATAGGCCATCAGCGCATGCGTGGAACCTTTGT |
| | | | GGCTCCTCTGCCGATCCATACTGCGGAACTCCTAGCCGCTTGTTTTGCTC |
| | | | GCAGCAGGTCTGGAGCGAAACTTATCGGGACAGATAATTCTGTCGTTCTC |
| | | | TCCCGGAAATATACATCATTTCCATGGCTGCTAGGCTGTGCTGCCAACTG |
| | | | GATCCTGCGAGGGACGTCCTTTGTCTACGTCCCGTCAGCGCTGAATCCTG |
| | | | CGGACGACCCGTCTCGGGGTCGCTTGGGGATCTATCGTCCCCTTCTCCGT |
| | | | CTGCCGTTCCAGCCGACCACGGGGCGCACCTCTCTTTACGCGGTCTCCCC |
| | | | GTCTGTGCCTTCTCATCTGCCGGACCGTGTGCACTTCGCTTCACCTCTGC |
| | | | ACGTCGCATGGAGACCACCGTGAACGCCCACCAAATCTTGCCCAAGGTCT |
| | | | TACATAAGAGGACTCTTGGACTCTCTGCAATGTCAACGACCGACCTTGAG |
| | | | GCATACTTCAAAGACTGTTTGTTTAAAGACTGGGAGGAGTTGGGGGAGGA |
| | | | GATTAGATTAAAGGTCTTTGTACTAGGAGGCTGTAGGCATAAATTGGTCT |
| | | | GCGCACCAGCACCATGCAACTTTTTCACCTCTGCCTAATCATCTCTTGTT |
| | | | CATGTCCTACTGTTCAAGCCTCCAAGCTGTGCCTTGGGTGGCTTTGGGGC |
| | | | ATGGACATTGACCCTTATAAAGAATTTGGAGCTACTGTGGAGTTACTCTC |
| | | | GTTTTTGCCTTCTGACTTCTTTCCTTCAGTAAGAGATCTTCTAGATACCG |
| | | | CCTCAGCTCTGTATCGGGATGCCTTAGAATCTCCTGAGCATTGTTCACCT |
| | | | CACCACACTGCACTCAGGCAAGCCATTCTTTGCTGGGGGGAACTAATGAC |
| | | | TCTAGCTACCTGGGTGGGTGTAAATTTGGAAGATCCAGCATCCAGGGACC |
| | | | TAGTAGTCAGTTATGTCAATACTAATATGGGCCTAAAGTTCAGGCAATTA |
| | | | TTGTGGTTTCACATTTCTTGTCTCACTTTTGGAAGAGAAACCGTCATAGA |
| | | | GTATTTGGTGTCTTTTGGAGTGTGGATTCGCACTCCTCCAGCTTATAGAC |
| | | | CACCAAATGCCCCTATCTTATCAACACTTCCGGAGAATACTGTTGTTAGA |
| | | | CGAAGAGGCAGGTCCCCTAGAAGAAGAACTCCCTCGCCTCGCAGACGAAG |
| | | | ATCTCAATCGCCGCGTCGCAGAAGATCTCAATCTCCAGCTTCCCAATGTT |
| | | | AGTATTCCTTGGACTCACAAGGTGGGAAATTTTACGGGGCTTTACTCTTC |
| | | | TACTATACCTGTCTTTAATCCTAACTGGAAAACTCCATCTTTTCCTGATA |
| | | | TTCATTTGCACCAGGACATTATTAACAAATGTGAACAATTTGTAGGTCCT |
| | | | CTAACAGTAAATGAAAAACGAAGATTAAACTTAGTCATGCCTGCTAGATT |
| | | | TTTTCCCATCTCTACGAAATATTTGCCCCTAGAGAAAGGTATAAAACCTT |
| | | | ATTATCCAGATAATGTAGTTAATCATTACTTCCAAACCAGACACTATTTA |
| | | | CATACCCTATGGAAGGCGGGCATCTTATATAAAAGAGAAACTACACGTAG |
| | | | CGCCTCATTTTGTGGGTCACCTTATTCTTGGGAACAAGAGCTACATCATG |
| | | | GGGCTTTCTTGGACGGTCCCTCTCGAATGGGGGAAGAATCATTCCACCAC |
| | | | CAATCCTCTGGGATTTTTTCCCGACCACCAGTTGGATCCAGCATTCAGAG |
| | | | CAAACACCAGAAATCCAGATTGGGACCACAATCCCAACAAAGACCACTGG |
| | | | ACAGAAGCCAACAAGGTAGGAGTGGGAGCATTCGGGCCGGGGTTCACTCC |
| | | | CCCACACGGAGGCCTTTTGGGGTGGAGCCCTCAGGCTCAAGGCATGCTAA |
| | | | AAACATTGCCAGCAGATCCGCCTCCTGCCTCCACCAATCGGCAGTCAGGA |
| | | | AGGCAGCCTACCCCAATCACTCCACCTTTGAGAGACACTCATCCTCAGGC |
| | | | CATGCAGTGGAA |
| GTF | KP718112 | 1481 | CTCAACTCAGTTCCACCAGGCTCTGTTGGATCCGAGGGTAAGGGCTCTGT |
| | | | ATTTTCCTGCTGGTGGCTCCAGTTCAGGGACACAGAACCCTGCTCCGACT |
| | | | ATTGCCTCTCTCACATCATCAATCTTCTCGAAGACTGGGGGCCCTGCCAT |
| | | | GAACATGGAGAACATCACATCAGGACTCCTAGGACCCCTGCTCGTGTTAC |
| | | | AGGCGGTGTGTTTCTTGTTGACAAAAATCCTCACAATACCACAGAGTCTA |
| | | | GACTCGTGGTGGACTTCTCTCAATTTTCTAGGGGGACTACCCGGGTGTCC |
| | | | TGGCCAAAATTCGCAGTCCCCAACCTCCAATCACTTACCAACCTCCTGTC |
| | | | CTCCAACTTGTCCTGGCTATCGTTGGATGTGTCTGCGGCGTTTTATCATC |
| | | | TTCCTCTTCATCCTGCTGCTATGCCTCATCTTCTTGTTGGTTCTTCTGGA |
| | | | CTACCAAGGTATGTTGCCCGTTTGTCCTCTACTTCCAGGATCCACGACCA |
| | | | CCAGCACGGGACCATGCAAAACCTGCACAACTCTTGCTCAAGGAACCTCT |
| | | | ATGTTTCCCTCCTGTTGCTGTTCCAAACCCTCGGACGGAAACTGCACCTG |
| | | | TATTCCCATCCCATCATCTTGGGCTTTAGGAAAATACCTATGGGAGTGGG |
| | | | CCTCAGCCCGTTTCTCCTGGCTCAGTTTACTAGTGCAATTTGTTCAGTGG |
| | | | TGCGTAGGGCTTTCCCCCACTGTCTGGCTTTTAGTTATATGGATGATCTG |
| | | | GTATTGGGGGCCAAATCTGTGCAGCATCTTGAGTCCCTTTATACCGCTGT |
| | | | TACCAATTTTCTGTTATCTGTGGGTATCCATTTAAATACTGCTAAAACAA |
| | | | AAAGATGGGGTTACTCCCTACATTTCATGGGTTATGTTATTGGTAGTTGG |
| | | | GGAACGTTACCCCAAGATCATATTGTACACAAAATCAAAGATTGTTTTCG |
| | | | GAAACTTCCTGTAAATCGTCCAATTGATTGGAAAGTTTGTCAACGCATTG |
| | | | TGGGTCTTTTGGGCTTTGCGGCCCCTTTCACCCAATGTGGTTATCCTGCT |
| | | | CTCATGCCTTTGTATACCTGTATTACTGCTAAACAGGCGTTTGTCTTTTC |
| | | | GCCAACTTACAAGGCCTTTCTCTGTAAACAATACATGAACCTTTACCCCG |
| | | | TTGCTCGGCAACGGCCAGGCCTGTGCCAAGTGTTTGCTGACGCAACCCCC |
| | | | ACTGGTTGGGGCTTGGCCATTGGCCATCAGCGCATGCGTGGAACCTTTGT |
| | | | GGCTCCTCTGCCGATCCATACTGCGGAACTCCTTGCAGCCTGTTTCGCTC |
| | | | GCAGCCGGTCTGGAGCGAACATTATCGGCACAGACAACTCTGTTGTCCTC |
| | | | TCTAGGAAGTACACCTCCTTTCCATGGCTGCTCGGTTGTGCTGCCAACTG |
| | | | GATCCTGCGCGGGACGTCCTTTGTTTACGTCCCGTCGGCGCTGAATCCCG |
| | | | CGGACGACCCCTCCCGGGGTCGCTTGGGGCTGTACCGCCCCCTTCTTCGT |
| | | | CTGCCGTTCCAGCCGACAACGGGTCGTACCTCTCTTTACGCGGACTCCCC |
| | | | GTCTGTTCCTTCTCATCTGCCGGACCGTGTGCACTTCGCTTCACCTCTGC |
| | | | ACGTCGCATGGAGACCACCGTGAACGCCCCCTGGAATCTGCCAACAGTCT |
| | | | TACATAAGAGGACTCTTGGACTTTCAGGACGGTCAATGACCTGGATCGAA |
| | | | GACTACATCAAAGACTGTGTATTTAAGGACTGGGAGGAGCTGGGGGAGGA |
| | | | GATCACGTTAAAGGTCTTTGTACTAGGAGGCTGTAGGCATAAATTGGTCT |
| | | | GTGCACCAGCACCATGCAACTTTTTCACCTCTGCCTAATCATCTTTTGTT |
| | | | CATGTCCCACTGTTCAAGCCTCCAAGCTGTGCCTTGGGTGGCTTTGGGGC |
| | | | ATGGACATTGACCCTTATAAAGAATTTGGAGCTTCTGTGGAATTGCTCTC |
| | | | TTTTTTGCCTTCTGATTTCTTCCCGTCTGTTCGGGACCTACTCGACACCG |
| | | | CTTCAGCCCTGTACCGGGATGCTCTAGAGTCACCGGAACATTGCACCCCC |
| | | | AATCATACCGCTCTCAGGCAAGCTATTTTGTGCTGGGGTGAGTTAATGAC |
| | | | TTTGGCTTCCTGGGTGGGTAATAATTTGGAAGACCCTGCAGCTAGGGATT |
| | | | TAGTAGTTAATTATGTCAACACTAATATGGGCCTAAAAATTAGACAACTG |
| | | | TTGTGGTTTCACATTTCCTGTCTTACTTTTGGAAGAGAAACAGTTCTTGA |
| | | | GTATTTGGTGTCCTTTGGAGTGTGGATTCGCACTCCACCTGCTTATAGAC |
| | | | CACCAAATGCCCCTATCCTATCCACACTTCCGGAAACTACTGTTGTTAGA |
| | | | CGACGAGGCAGGTCCCCTAGAAGAAGAACTCCCTCGCCTCGCCGACGAAG |
| | | | GTCTCAATCGCCGCGTCGCAGAAGATCTCAATCTCCAGCTTCCCAATGTT |
| | | | AGTATTCCTTGGACTCATAAGGTGGGAAATTTTACGGGGCTCTACTCTTC |
| | | | TACTGTACCTGCTTTCAATCCTAACTGGTTAACTCCTTCTTTTCCTGATA |
| | | | TTCATTTACATCAGGATCTGATATCTAAATGTGAACAATTTGTAGGCCCG |
| | | | CTCACTAAAAATGAATTGAGAAGATTAAAATTGGTCATGCCAGCTAGATT |
| | | | TTATCCTAAGGTTACCAAATACTTTCCTATGGAGAAAGGGATTAAACCCT |
| | | | ATTATCCTGAGCATGCAGTTAATCATTATTTTAAAACAAGACATTATTTG |
| | | | CATACTTTATGGAAGGCGGGAATCTTATATAAGAGAGAATCCACACGTAG |
| | | | CGCCTCATTTTGTGGGTCACCATATTCCTGGGAACAAGAGCTACAGCATG |
| | | | GGAGCACCTCTCTCAACGACAAGAAGGGGCATGGGACTGAATCTTTCTGT |
| | | | GCCCAATCCTCTGGGCTTCTTGCCAGACCATCAGCTGGATCCGCTATTCA |
| | | | GAGCAAATTCCAGCAGTCCCGACTGGGACTTCAACACAAACAAGGACAGT |
| | | | TGGCCAATGGCAAACAAGGTAGGAGTGGGAGGCTACGGTCCAGGGTTCAC |
| | | | ACCCCCACACGGTGGCCTGCTGGGGTGGAGCCCTCAGGCACAGGGTGTTT |
| | | | TAACAACCTTGCCAGCAGATCCGCCTCCTGCTTCCACCAATCGGCGGTCC |
| | | | GGGAGAAAGCCAACCCCAGTCTCTCCACCTCTAAGAGACACACATCCACA |
| | | | GGCCATGCAGTGGAA |
| GTG | KX264500 | 1482 | CTCTACAGCATTCCACCAAGCTCTACAAAATCCCAAAGTCAGGGGCCTGT |
| | | | ATTTTCCTGCTGGTGGCTCCAGTTCAGGGATAGTGAACCCTGTTCCGACT |
| | | | ATTGCCTCTCACATCTCGTCAATCTTCTCCAGGATTGGGGACCCTGCACC |
| | | | GAACATGGAGAACATCACATCAGGATTCCTAGGACCCCTGCTCGTGTTAC |
| | | | AGGCGGGGTTTTTCTTGTTGACAAGAATCCTCACAATACCGCAGAGTCTA |
| | | | GACTCGTGGTGGACTTCTCTCAATTTTCTAGGGGGAGTGCCCGTGTGTCC |
| | | | TGGCCTAAATTCGCAGTCCCCAACCTCCAATCACTCACCAATCTCCTGTC |
| | | | CTCCAACTTGTCCTGGCTATCGCTGGATGTGTCTGCGGCGTTTTATCATA |
| | | | TTCCTCTTCATCCTGCTGCTATGCCTCATCTTCTTGTTGGTTCTTCTGGA |
| | | | CTATCAAGGTATGTTGCCCGTTTGTCCTCTGATTCCAGGATCCTCGACCA |
| | | | CCAGTACGGGACCCTGCAAAACCTGCACGACTCCTGCTCAAGGCAACTCT |
| | | | ATGTATCCCTCATGTTGCTGTACAAAACCTTCGGACGGAAATTGCACCTG |
| | | | TATTCCCATCCCATCATCTTGGGCTTTCGCAAAATACCTATGGGAGTGGG |
| | | | CCTCAGTCCGTTTCTCTTGGCTCAGTTTACTAGTGCCATTTGTTCAGTGG |
| | | | TTCGTAGGGCTTTCCCCCACTGTCTGGCTTTCAGCTATATGGATGATGTG |
| | | | GTATTGGGGGCCAAATCTGTACAACATCTTGAGTCCCTTTATACCGCTGT |
| | | | TACCAATTTTCTTTTGTCTTTGGGTATACATCTAAACCCTAACAAAACAA |
| | | | AAAGATGGGGTTATTCCTTAAATTTTATGGGATATGTAATTGGAAGTTGG |
| | | | GGTACTTTGCCACAAGAACACATCACACAGAAAATTAAGCAATGTTTTCG |
| | | | GAAACTCCCTGTTAACAGGCCAATTGATTGGAAAGTCTGTCAACGAATAA |
| | | | CTGGTCTGTTGGGTTTCGCTGCTCCTTTTACCCAATGTGGTTACCCTGCC |
| | | | TTAATGCCTTTATATGCATGTATACAAGCTAAGCAGGCTTTTACTTTCTC |
| | | | GCCAACTTATAAGGCCTTTCTCTGTAAACAATACATGAACCTTTACCCCG |
| | | | TTGCTAGGCAACGGCCCGGTCTGTGCCAAGTGTTTGCTGACGCAACCCCC |
| | | | ACTGGTTGGGGCTTGGCCATCGGCCATCAGCGCATGCGTGGAACCTTTGT |
| | | | GGCTCCTCTGCCGATCCATACTGCGGAACTCCTAGCTGCTTGTTTTGCTC |
| | | | GCAGCCGGTCTGGAGCAAAACTCATTGGGACTGACAATTCTGTCGTCCTT |
| | | | TCTCGGAAATATACATCCTTTCCATGGCTGCTAGGCTGTGCTGCCAACTG |
| | | | GATCCTTCGCGGGACGTCCTTTGTTTACGTCCCGTCAGCGCTGAATCCAG |
| | | | CGGACGACCCCTCCCGGGGCCGTTTGGGGCTCTGTCGCCCCCTTCTCCGT |
| | | | CTGCCGTTCCTGCCGACCACGGGGCGCACCTCTCTTTACGCGGTCTCCCC |
| | | | GTCTGTTCCTTCTCATCTGCCGGACCGTGTGCACTTCGCTTCACCTCTGC |
| | | | ACGTTACATGGAAACCGCCATGAACACCTCTCATCATCTGCCAAGGCAGT |
| | | | TATATAAGAGGACTCTTGGACTGTTTGTTATGTCAACAACCGGGGTGGAG |
| | | | AAATACTTCAAGGACTGTGTTTTTGCTGAGTGGGAAGAATTAGGCAATGA |
| | | | GTCCAGGTTAATGACCTTTGTATTAGGAGGCTGTAGGCATAAATTGGTCT |
| | | | GCGCACCAGCACCATGTAACTTTTTCACCTCTGCCTAATCATCTCTTGTT |
| | | | CATGTCCTACTGTTCAAGCCTCCAAGCTGTGCCTTGGGTGGCTTTAGGGC |
| | | | ATGGATAGAACAACTTTGCCATATGGCCTTTTTGGCTTAGACATTGACCC |
| | | | TTATAAAGAATTTGGAGCTACTGTGGAGTTGCTCTCGTTTTTGCCTTCTG |
| | | | ACTTTTTCCCGTCTGTTCGTGATCTTCTCGACACCGCTTCAGCTTTGTAC |
| | | | CGGGAATCCTTAGAGTCCTCTGATCATTGTTCGCCTCACCATACAGCACT |
| | | | CAGGCAAGCAATCCTGTGCTGGGGTGAGTTGATGACTCTAGCTACCTGGG |
| | | | TGGGTAATAATTTGGAAGATCCAGCATCCAGAGATTTGGTGGTCAATTAT |
| | | | GTTAATACTAATATGGGTTTAAAAATCAGGCAACTATTGTGGTTTCACAT |
| | | | TTCCTGTCTTACTTTTGGGAGAGAAACCGTTCTTGAGTATTTGGTGTCTT |
| | | | TTGGAGTGTGGATTCGCACTCCTCCTGCTTATAGACCACCAAATGCCCCT |
| | | | ATCCTATCAACACTTCCGGAGACTACTGTTGTTAGACGAAGAGGCAGGTC |
| | | | CCCTCGAAGAAGAACTCCCTCGCCTCGCAGACGAAGATCTCAATCGCCGC |
| | | | GTCGCAGAAGATCTGCATCTCCAGCTTCCCAATGTTAGTATTCCTTGGAC |
| | | | TCACAAGGTGGGAAACTTTACGGGGCTGTATTCTTCTACTATACCTGTCT |
| | | | TTAATCCTGATTGGCAAACTCCTTCTTTTCCAAATATCCATTTGCATCAA |
| | | | GACATTATAACTAAATGTGAACAATTTGTGGGCCCTCTCACAGTAAATGA |
| | | | GAAACGAAGATTAAAACTAGTTATGCCTGCCAGATTTTTCCCAAACTCTA |
| | | | CTAAATATTTACCATTAGACAAAGGTATCAAACCGTATTATCCAGAAAAT |
| | | | GTAGTTAATCATTACTTCCAGACCAGACATTATTTACATACCCTTTGGAA |
| | | | GGCGGGTATTCTATATAAGAGAGAAACATCCCGTAGCGCTTCATTTTGTG |
| | | | GGTCACCATATACTTGGGAACAAGATCTACAGCATGGGGCTTTCTTGGAC |
| | | | GGTCCCTCTCGAGTGGGGAAAGAACCTTTCCACCAGCAATCCTCTAGGAT |
| | | | TCCTTCCCGATCACCAGTTGGACCCAGCATTCAGAGCAAATACCAACAAT |
| | | | CCAGATTGGGACTTCAATCCCAAAAAGGACCCTTGGCCAGAGGCCAACAA |
| | | | GGTAGGAGTTGGAGCCTATGGACCCGGGTTCACCCCTCCACACGGAGGCC |
| | | | TTTTGGGGTGGAGCCCTCAGTCTCAGGGCACACTAACAACTTTGCCAGCA |
| | | | GATCCGCCTCCTGCCTCCACCAATCGTCAGTCAGGGAGGCAGCCTACTCC |
| | | | CATCTCTCCACCACTAAGAGACAGTCATCCTCAGGCCATGCAGTGGAA |
| GTH | KX264501 | 1483 | CTCAACACAGTTCCACCAAGCACTGTTGGATCCGAGAGTAAGGGGTCTGT |
| | | | ATTTTCCTGCTGGTGGCTCCAGTTCAGAAACACAGAACCCTGCTCCGACT |
| | | | ATTGCCTCTCTCACATCATCAATCTTCTCGAAGACTGGGGACCCTGCTAT |
| | | | GAACATGGAGAACATCACATCAGGACTCCTAGGACCCCTTCTCGTGTTAC |
| | | | AGGCGGTGTGTTTCTTGTTGACAAAAATCCTCACAATACCACAGAGTCTA |
| | | | GACTCGTGGTGGACTTCTCTCAATTTTCTAGGGGTACCACCCGGGTGTCC |
| | | | TGGCCAAAATTCGCAGTCCCCAATCTCCAATCACTTACCAACCTCCTGTC |
| | | | CTCCAACTTGTCCTGGCTATCGTTGGATGTGTCTGCGGCGTTTTATCATC |
| | | | TTCCTCTTCATCCTGCTGCTATGCCTCATCTTCTTGTTGGTTCTTCTGGA |
| | | | CTATCAAGGTATGTTGCCCGTGTGTCCTCTACTTCCAGGATCTACAACCA |
| | | | CCAGCACGGGACCCTGCAAAACCTGCACCACTCTTGCTCAAGGAACCTCT |
| | | | ATGTTTCCCTCCTGCTGCTGTACCAAACCTTCGGACGGAAATTGCACCTG |
| | | | TATTCCCATCCCATCATCTTGGGCTTTCGGAAAATACCTATGGGAGTGGG |
| | | | CCTCAGCCCGTTTCTCTTGGCTCAGTTTACTAGTGCAATTTGTTCAGTGG |
| | | | TGCGTAGGGCTTTCCCCCACTGTCTGGCTTTTAGTTATATGGATGATTTG |
| | | | GTATTGGGGGCCAAATCTGTGCAGCATCTTGAGTCCCTTTATACCGCTGT |
| | | | TACCAATTTTTTGTTATCTGTGGGCATCCATTTGAACACAGCTAAAACAA |
| | | | AATGGTGGGGTTATTCCTTACACTTTATGGGTTATATAATTGGGAGTTGG |
| | | | GGGACCTTGCCTCAGGAACATATTGTGCATAAAATCAAAGATTGCTTTCG |
| | | | CAAACTTCCCGTGAATAGACCCATTGATTGGAAGGTTTGTCAACGCATTG |
| | | | TGGGTCTTTTGGGCTTTGCAGCCCCTTTTACTCAATGTGGTTATCCTGCT |
| | | | CTCATGCCCTTGTATGCCTGTATTACCGCTAAGCAGGCTTTTGTTTTCTC |
| | | | GCCAACTTACAAGGCCTTTCTCTGTAAACAATACATGAACCTTTACCCCG |
| | | | TTGCTCGGCAACGGCCAGGCCTTTGCCAAGTGTTTGCTGACGCAACCCCC |
| | | | ACTGGCTGGGGCTTGGCGATTGGCCATCAGCGCATGCGCGGAACCTTTGT |
| | | | GGCTCCTCTGCCGATCCATACTGCGGAACTCCTAGCAGCTTGTTTCGCTC |
| | | | GCAGCAGGTCTGGAGCGGACATTATCGGCACTGACAACTCCGTTGTCCTT |
| | | | TCTCGGAAGTACACCTCCTTCCCATGGCTGCTAGGCTGTGCTGCCAACTG |
| | | | GATCCTGCGCGGGACGTCCTTTGTCTACGTCCCGTCGGCGCTGAATCCTG |
| | | | CGGACGACCCCTCTCGTGGTCGCTTGGGGCTCTGCCGCCCTCTTCTCCGC |
| | | | CTGCCGTTCCGGCCGACGACGGGTCGCACCTCTCTTTACGCGGACTCCCC |
| | | | GCCTGTGCCTTCTCATCTGCCGGCCCGTGTGCACTTCGCTTCACCTCTGC |
| | | | ACGTCGCATGGAGACCACCGTGAACGCCCCTTGGAACTTGCCAACAACCT |
| | | | TACATAAGAGGACTCTTGGACTTTCGCCCCGGTCAACGACCTGGATTGAG |
| | | | GAATACATCAAAGACTGTGTATTTAAGGACTGGGAGGAGTCGGGGGAGGA |
| | | | GTTGAGGTTAAAGGTCTTTGTATTAGGAGGCTGTAGGCATAAATTGGTCT |
| | | | GTTCACCAGCACCATGCAACTTTTTCACCTCTGCCTAATCATCTTTTGTT |
| | | | CATGTCCCACTGTTCAAGCCTCCAAGCTGTGCCTTGGGTGGCTTTGGGGC |
| | | | ATGGACATTGACCCTTATAAAGAATTTGGAGCTTCTGTGGAGTTACTCTC |
| | | | ATTTTTGCCTTCTGACTTCTTCCCGTCTGTCCGGGACCTACTCGACACCG |
| | | | CTTCAGCCCTCTACCGAGATGCCTTAGAATCACCCGAACATTGCACCCCC |
| | | | AACCACACTGCTCTCAGGCAAGCTATTTTGTGCTGGGGTGAGTTGATGAC |
| | | | CTTGGCTTCCTGGGTGGGCAATAATTTAGAGGATCCTGCAGCAAGAGATC |
| | | | TAGTAGTTAATTATGTCAATACTAACATGGGCCTAAAAATTAGACAATTA |
| | | | TTATGGTTTCACATTTCCTGCCTTACATTTGGAAGAGAAACTGTGCTTGA |
| | | | GTATTTGGTGTCTTTTGGAGTGTGGATTCGCACTCCACCTGCTTATAGAC |
| | | | CACCAAATGCCCCTATCCTATCAACACTTCCGGAGACTACTGTTGTTAGA |
| | | | CAACGAGGCAGGGCCCCTAGAAGAAGAACTCCCTCGCCTCGCAGACGAAG |
| | | | ATCTCAATCACCGCGTCGCAGAAGATCTCAATCTCCAGCTTCCCAATGTT |
| | | | AGTATTCCTTGGACTCATAAGGTGGGAAACTTTACCGGTCTTTACTCCTC |
| | | | TACTATACCTGTTTTCAATCCTGACTGGTTAACTCCTTCTTTTCCTGACA |
| | | | TTCACTTGCATCAAGATCTGATACAAAAATGTGAACAATTTGTAGGCCCA |
| | | | CTCACTACAAATGAAAGGAGACGATTGAAACTAATTATGCCAGCTAGGTT |
| | | | TTATCCCAAAGTTACTAAATACTTCCCTTTGGATAAAGGTATTAAGCCTT |
| | | | ACTATCCAGAGAATGTGGTTAATCATTACTTTAAAACTAGACATTATTTA |
| | | | CATACTTTGTGGAAGGCAGGAATTCTATATAAGAGAGAATCCACACATAG |
| | | | CGCCTCATTTTGTGGGTCACCATATTCCTGGGAACAAGAGCTACAGCATG |
| | | | GGAGCACCTCTCTCAACGGCGAGAAGGGGCATGGGACAGAATCTTTCTGT |
| | | | GCCCAATCCTCTGGGATTCTTTCCAGACCACCAGTTGGATCCACTATTCA |
| | | | GAGCAAATTCCAGCAGTCCCGATTGGGACTTCAACACAAACAAGGACAAT |
| | | | TGGCCAATGGCAAACAAGGTAGGAGTGGGAGGCTTCGGTCCAGGGTTCAC |
| | | | ACCCCCACACGGTGGCCTTCTGGGGTGGAGCCCTCAGGCACAGGGCATTC |
| | | | TGACAACCTCGCCACCAGATCCACCTCCTGCTTCCACCAATCGGAGGTCA |
| | | | GGAAGAAAGCCAACCCCAGTCTCTCCACCTCTAAGGGACACACATCCACA |
| | | | GGCCATGCAGTGGAA |

In certain embodiments, the HBV nucleic acid is the sequence set forth in GENBANK Accession No. U95551.1 (incorporated herein as SEQ ID NO: 3). In some embodiments, the modified multi-segmented antisense oligonucleotides target a sequence recited in SEQ ID NO: 3 or a portion thereof. In some embodiments, the modified multi-segmented antisense oligonucleotides target a sequence at positions 1583-1602 of SEQ ID NO: 3.

### PCSK9

Proprotein Convertase Subtilisin/Kexin Type 9 (PCSK9) may also be referred to as Subtilisin/Kexin-Like Protease PC9. Human PCSK9 has a cytogenetic location of 1p32.3 and the genomic coordinates are on Chromosome 1 on the forward strand at position 55,039,548-55,064,852. Human PCSK9 has a NCBI gene ID of 255738, Ref Seq Accession No. of NM_174936.4, Ref Seq Accession No. of NP_777596.2 and Ensembl Gene ID of ENSG00000169174.

The activity of PCSK9 is primarily in the liver and PCSK9 is associated with Dyslipidemias, PCSK9-related familial hypercholesterolemia, hypercholesterolemia (familial), gastric papillary adenocarcinoma, homozygous familial hypercholesterolemia, and nasopharyngitis, PCSK9-related familial hypercholesterolemia is an inherited disease (autosomal dominant) where the body develops dangerously blood cholesterol levels due to the lack of a receptor for the low-density lipoprotein cholesterol. PCSK9-related familial hypercholesterolemia affects between 1 in 500 heterozygotic and 1 in 1,000,000 homozygotic people worldwide and is more common in Afrikaner, French Canadians, Lebanese Christians, and Finns populations. Common symptoms of PCSK9-related familial hypercholesterolemia include elevated circulating cholesterol contained in either low-density lipoproteins alone or also in very-low-density lipoproteins. Current treatments of PCSK9-related familial hypercholesterolemia include administration of statins to inhibit hydroxymethylglutaryl CoA reductase (HMG-CoA-reductase) in the liver. Another option for treating PCSK9-related familial hypercholesterolemia is ezetimibe to inhibit cholesterol absorption in the gut.

In some embodiments, the PCSK9 nucleic acid is the sequence set forth in NCBI gene ID of NM_174936.4 or a portion thereof.

In some embodiments, the PCSK9 target comprises the sequence recited in SEQ ID NOS: 1454, 1458, or 1472 or a portion thereof or a variant thereof. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary SEQ ID NO: 1454, 1458, or 1472.

In some embodiments, the modified multi-segmented antisense oligonucleotide comprises a nucleobase sequence of SEQ ID NO: 1432, 1436, or 1450. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% identical to SEQ ID NO: 1432, 1436, or 1450.

### DMPK

DM1 protein kinase (DMPK) may also be referred to as DM, DM1, DMK, MDPK, DM1PK, MT-PK. Human DMPK has a cytogenetic location of 19q13.32 and the genomic coordinates are on Chromosome 19 on the reverse strand at position 45,769,709-45,782,490. Human DMPK has a NCBI gene ID of 1760, Ref Seq Accession No. of NM_004409.5, Ref Seq Accession No. of NP_004400.4 and Ensembl Gene ID of ENSG00000104936.

The activity of DMPK is primarily in the muscle and DMPK is associated with Myotonic Dystrophy 1 and Adult-Onset Steinert Myotonic Dystrophy. The genetic defect in DM1 results from an amplified trinucleotide repeat in the 3-prime untranslated region of a protein kinase gene. Disease severity varies with the number of repeats: normal individuals have 5 to 37 repeats, mildly affected persons have 50 to 150 repeats, patients with classic DM have 100 to 1,000 repeats, and those with onset at birth can have more than 2,000 repeats. The disorder shows genetic anticipation, with expansion of the repeat number dependent on the sex of the transmitting parent. Alleles of 40 to 80 repeats are usually expanded when transmitted by males, whereas only alleles longer than 80 repeats tend to expand in maternal transmissions. Repeat contraction events occur 4.2 to 6.4% of the time. The prevalence ratio of DM1 is over 1/1000 in specific population and 1-9/100000 people worldwide, especially in Europe, Italy, Japan, Ireland, Norway, United States. DM1 is characterized mainly by myotonia, muscular dystrophy, cataracts, hypogonadism, frontal balding, and ECG changes. Current approved drugs for DM1 include Methylphenidate, Dopamine, Lamotrigine, Mexiletine, Metformin.

In some embodiments, the DMPK nucleic acid is the sequence set forth in NCBI gene ID of NM_004409.5 or a portion thereof.

In some embodiments, the DMPK target comprises the sequence recited in SEQ ID NO: 1455, 1459, 1460, or 1461 or a portion thereof or a variant thereof. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary to SEQ ID NO: 1455, 1459, 1460, or 1461.

In some embodiments, the modified multi-segmented antisense oligonucleotide has a nucleobase sequence of SEQ ID NO: 1433, 1437, 1438, or 1439. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% identical to SEQ ID NO: 1433, 1437, 1438, or 1439.

### TTR

Transthyretin (TTR) may also be referred to as CTS, TTN, ATTR, CTS1, PALB, TBPA, HEL111, HsT2651. Human TTR has a cytogenetic location of 18q12.1 and the genomic coordinates are on Chromosome 18 on the forward strand at position 31,591,877-31,598,821. Human TTR has a NCBI gene ID of 7276, Ref Seq Accession No. of NM_000371.4, Ref Seq Accession No. of NP_000362.1 and Ensembl Gene ID of ENSG00000118271.

Transthyretin that is synthesized in the liver is secreted into the blood, whereas transthyretin originating in the choroid plexus is destined for the CSF (cerebrospinal fluid). Transthyretin is associated with both local and systemic amyloidosis, a disorder characterized by extracellular systemic deposition of mutated or wild-type transthyretin as amyloid fibrils. Senile systemic amyloidosis is a sporadic disorder resulting from the extracellular deposition of wild-type transthyretin fibrils in cardiac and other tissues. Over 80 mutations in transthyretin are associated with familial amyloidotic polyneuropathy and cardiomyopathy. The variants Val122Ile, Val30Met and Gluo.1Lys are common amyloidogenic mutations worldwide. In most of these cases, inheritance is autosomal dominant. The prevalence ratio of Val30Met Amyloidosis is 6-9/10000 in Portugal, 1-9/1000000 in Japan and Cyprus. Patients with transthyretin amyloidosis typically present with polyneuropathy, carpal tunnel syndrome, autonomic insufficiency, cardiomyopathy, and gastrointestinal features, occasionally accompanied by vitreous opacities and renal insufficiency. In later stages of the disease severe diarrhea with malabsorption, cachexia, incapacitating neuropathy, severe cardiac disturbances, and marked orthostatic hypotension dominate the clinical picture. Death usually occurs 5 to 15 years after onset of symptoms. The FDA has approved vutrisiran (Amvuttra), patisiran (Onpattro) and inotersen (Tegsedi) for treatment of polyneuropathy caused by hATTR in adults. Tafamidis (Vyndamax) and tafamidis meglumine (Vyndagel) are FDA approved for transthyretin-mediated amyloid cardiomyopathy (ATTR-CM).

In some embodiments, the TTR nucleic acid is the sequence set forth in NCBI gene ID of NM_000371.4 or a portion thereof.

In some embodiments, the TTR target comprises the sequence recited in SEQ ID NO: 1456, 1462, or 1474 or a portion thereof or a variant thereof. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary to SEQ ID NO: 1456, 1462, or 1474.

In some embodiments, the modified multi-segmented antisense oligonucleotide has a nucleobase sequence of SEQ ID NO: 1434, 1440, or 1452. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% identical to SEQ ID NO: 1434, 1440, or 1452.

### AAT

Serpin family A member 1 (SERPINA1) may also be referred to as PI, A1A, AAT, PI1, A1AT, nNIF, PRO2275, alpha1AT. Human SERPINA1 has a cytogenetic location of 14q32.13 and the genomic coordinates are on Chromosome 14 on the reverse strand at position 94,376,747-94,390,635. Human SERPINA1 has a NCBI gene ID of 5265, Ref Seq Accession No. of NM_000295.5, Ref Seq Accession No. of NP_000286.3 and Ensembl Gene ID of ENSG00000197249.

AAT is predominantly produced by hepatocytes and secreted into blood; but low levels of AAT expression are also detected in the lung and in the gut. Diseases associated with SERPINA1 include Alpha-1-Antitrypsin Deficiency and Hemorrhagic Disease Due To Alpha-1-Antitrypsin Pittsburgh Mutation. Alpha-1 antitrypsin deficiency is an inherited disorder that may cause lung disease and liver disease. Alpha-1 antitrypsin deficiency occurs worldwide, but its prevalence varies by population. This disorder affects about 1 in 1,500 to 3,500 individuals with European ancestry. It is uncommon in people of Asian descent. Many individuals with alpha-1 antitrypsin deficiency are likely undiagnosed, particularly people with a lung condition called chronic obstructive pulmonary disease (COPD). COPD can be caused by alpha-1 antitrypsin deficiency; however, the alpha-1 antitrypsin deficiency is often never diagnosed. Some people with alpha-1 antitrypsin deficiency are misdiagnosed with asthma. Treatment of lung disease may include bronchodilators, inhaled steroids, and, when infections occur, antibiotics. Intravenous infusions of the A1AT protein or in severe disease lung transplantation may also be recommended. In those with severe liver disease liver transplantation may be an option. Avoiding smoking and getting vaccinated for influenza, pneumococcus, and hepatitis is also recommended.

In some embodiments, the AAT nucleic acid is the sequence set forth in NCBI gene ID of NM_000295.5 or a portion thereof.

In some embodiments, the AAT target comprises the sequence recited in SEQ ID NO: 1457 or 1463 or a portion thereof or a variant thereof. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary SEQ ID NO: 1457 or 1463.

In some embodiments, the modified multi-segmented antisense oligonucleotide has a nucleobase sequence of SEQ ID NO: 1435 or 1441. In some embodiments, the modified multi-segmented antisense oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% identical to SEQ ID NO: 1435 or 1441.

As a general matter, the target of the modified multi-segmented antisense oligonucleotides of the disclosures are the transcripts of genes. In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for a target can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense oligonucleotide sequences that may hybridize in a non-specific manner to sequences other than a selected target nucleic acid (i.e., non-target or off-target sequences).

### Hybridization

In some embodiments, hybridization occurs between an antisense oligonucleotide disclosed herein and a target nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense oligonucleotides provided herein are specifically hybridizable with a target nucleic acid.

### Complementarity

An antisense oligonucleotide and a target nucleic acid are complementary to each other when a sufficient number of nucleobases of the antisense oligonucleotide can hydrogen bond with the corresponding nucleobases of the target nucleic acid, such that a desired effect will occur (e.g., antisense inhibition of a target nucleic acid).

Non-complementary nucleobases between an antisense oligonucleotide and a target nucleic acid may be tolerated provided that the antisense oligonucleotide remains able to specifically hybridize to a target nucleic acid. Moreover, an antisense oligonucleotide may hybridize over one or more segments of a target nucleic acid such that intervening or adjacent segments are not involved in the hybridization event (e.g., a loop structure, mismatch or hairpin structure).

In certain embodiments, the antisense oligonucleotides provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a target nucleic acid, a target region, target segment, or specified portion thereof. Percent complementarity of an antisense oligonucleotide with a target nucleic acid can be determined using routine methods.

For example, an antisense oligonucleotide in which 18 of 20 nucleobases of the antisense oligonucleotide are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligonucleotide which is 18 nucleobases in length having four noncomplementary nucleobases which are flanked by two regions of complete complementarity with the target nucleic acid would have 77.8% overall complementarity with the target nucleic acid and would thus fall within the scope of the present invention. Percent complementarity of an antisense oligonucleotide with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense oligonucleotides provided herein, or specified portions thereof, are fully complementary (i.e. 100% complementary) to a target nucleic acid, or specified portion thereof. As used herein, “fully complementary” means each nucleobase of an antisense oligonucleotide is capable of precise base pairing with the corresponding nucleobases of a target nucleic acid. For example, a 20 nucleobase antisense oligonucleotide is fully complementary to a target sequence that is 400 nucleobases long, so long as there is a corresponding 20 nucleobase portion of the target nucleic acid that is fully complementary to the antisense oligonucleotide. Fully complementary can also be used in reference to a specified portion of the first and/or the second nucleic acid. For example, a 20 nucleobase portion of a 30 nucleobase antisense oligonucleotide can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense oligonucleotide. At the same time, the entire 30 nucleobase antisense oligonucleotide may or may not be fully complementary to the target sequence, depending on whether the remaining 10 nucleobases of the antisense oligonucleotide are also complementary to the target sequence.

The location of a non-complementary nucleobase may be at the 5′ end or 3′ end of the antisense oligonucleotide. Alternatively, the non-complementary nucleobase or nucleobases may be at an internal position of the antisense oligonucleotide. When two or more non-complementary nucleobases are present, they may be contiguous (i.e. linked) or non-contiguous. In one embodiment, a non-complementary nucleobase is located in the wing segment of a gapmer antisense oligonucleotide. In one embodiment, a non-complementary nucleobase is located in the wing segment of a multi-segmented antisense oligonucleotide.

The antisense oligonucleotides provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense oligonucleotide. In certain embodiments, the antisense oligonucleotides, are complementary to at least an 8 nucleobase portion of a target segment. In certain embodiments, the antisense oligonucleotides are complementary to at least a 9 nucleobase portion of a target segment. In certain embodiments, the antisense oligonucleotides are complementary to at least a 10 nucleobase portion of a target segment. In certain embodiments, the antisense oligonucleotides are complementary to at least an 11 nucleobase portion of a target segment. In certain embodiments, the antisense oligonucleotides are complementary to at least a 12 nucleobase portion of a target segment. In certain embodiments, the antisense oligonucleotides are complementary to at least a 13 nucleobase portion of a target segment. In certain embodiments, the antisense oligonucleotides are complementary to at least a 14 nucleobase portion of a target segment. In certain embodiments, the antisense oligonucleotides are complementary to at least a 15 nucleobase portion of a target segment. Also contemplated are antisense oligonucleotides that are complementary to at least a 9, 10, 11, 12, 13, 14, 15, 16, 17, 18, 19, 20, or more nucleobase portion of a target segment, or a range defined by any two of these values.

### Identity

The antisense oligonucleotides provided herein may also have a defined percent identity to a particular nucleotide sequence (i.e. SEQ ID NOs: 11-1453 or 5000-30983, or oligonucleotide, or portion thereof). As used herein, an antisense oligonucleotide is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense oligonucleotides described herein as well as oligonucleotides having non-identical bases relative to the antisense oligonucleotides provided herein also are contemplated. The non-identical bases may be adjacent to each other or dispersed throughout the antisense oligonucleotide. Percent identity of an antisense oligonucleotide is calculated according to the number of bases that have identical base pairing relative to the sequence to which it is being compared.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the terms “substantially complementary” or “partially complementary” mean that two nucleic acid sequences are complementary at least at about 50%, 60%, 70%, 80% or 90% of their nucleotides.

In some embodiments, the two nucleic acid sequences can be complementary at least at 85%, 90%, 95%, 96%, 97%, 98%, 99% or more of their nucleotides. In some embodiments, the two nucleic acid sequences can be between 60% to 100% complementary, between 70% to 100% complementary, between 80% and 100% complementary, between 90% and 100% complementary, between 60% to 90% complementary, between 60% to 80% complementary, between 60% and 70% complementary, between 70% and 90% complementary, between 70% and 80% complementary, between 80% and 100% complementary, or between 80% and 90% complementary.

The terms “substantially complementary” and “partially complementary” can also mean that two nucleic acid sequences can hybridize under high stringency conditions, and such conditions are well known in the art.

As used herein, the term “identity” means that sequences are compared with one another as follows. In order to determine the percentage identity of two nucleic acid sequences, the sequences can first be aligned with respect to one another in order subsequently to make a comparison of these sequences possible. For this e.g. gaps can be inserted into the sequence of the first nucleic acid sequence and the nucleotides can be compared with the corresponding position of the second nucleic acid sequence. If a position in the first nucleic acid sequence is occupied by the same nucleotide as is the case at a position in the second sequence, the two sequences are identical at this position. The percentage identity between two sequences is a function of the number of identical positions divided by the number of all the positions compared in the sequences investigated.

A “percent identity” for aligned segments of a test sequence and a reference sequence is the percent of identical components which are shared by the two aligned sequences divided by the total number of components in reference sequence segment, i.e., the entire reference sequence or a smaller defined part of the reference sequence.

The percentage identity of two sequences can be determined with the aid of a mathematical algorithm. An example of a mathematical algorithm which can be used for comparison of two sequences is the algorithm of Karlin et al. (1993), PNAS USA, 90:5873-5877. Such an algorithm is integrated in the NBLAST program, with which sequences which have a desired identity to the sequences of the present invention can be identified. In order to obtain a gapped alignment, as described here, the “Gapped BLAST” program can be used, as is described in Altschul et al. (1997), Nucleic Acids Res, 25:3389-3402. If BLAST and Gapped BLAST programs are used, the preset parameters of the particular program (e.g. NBLAST) can be used. The sequences can be aligned further using version 9 of GAP (global alignment program) of the “Genetic Computing Group” using the preset (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of agreements as a percentage content of the nucleic acids in the sequence claimed. The methods described for determination of the percentage identity of two nucleic acid sequences can also be used correspondingly, if necessary, on the coded amino acid sequences.

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Percent identity can be 70% identity or greater, e.g., at least 70% identity, at least 75% identity, at least 80% identity, at least 85% identity, at least 90% identity, at least 95% identity, at least 98% identity, at least 99% identity or 100% identity.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense oligonucleotides encompass substitutions or changes to internucleoside linkages, sugar moieties, or nucleobases. Modified antisense oligonucleotides are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense oligonucleotides that have such chemically modified nucleosides.

### Modified Internucleoside Linkages

The naturally occurring internucleoside linkage of RNA and DNA is a 3′ to 5′ phosphodiester linkage. Antisense oligonucleotides having one or more modified, i.e. non-naturally occurring, internucleoside linkages are often selected over antisense oligonucleotides having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense oligonucleotides targeted to a target nucleic acid comprise one or more modified internucleoside linkages. In certain embodiments, the modified internucleoside linkages are phosphorothioate linkages. In certain embodiments, each internucleoside linkage of an antisense oligonucleotide is a phosphorothioate internucleoside linkage. In some embodiments, a modified multi-segmented antisense oligonucleotide of the disclosure comprises one or more modified internucleoside linkages, wherein the modified internucleoside linkage is a phosphorodiamidate linkage between morpholino nucleoside mimetics.

### Modified Sugar Moieties

Antisense oligonucleotides provided herein can optionally contain one or more nucleosides wherein the sugar group has been modified. Such sugar modified nucleosides may impart enhanced nuclease stability, increased binding affinity, or some other beneficial biological property to the antisense oligonucleotides. In certain embodiments, nucleosides comprise a chemically modified ribofuranose ring moiety. Examples of chemically modified ribofuranose rings include, without limitation, addition of substituent groups (including 5′ and 2′ substituent groups); bridging of non-geminal ring atoms to form bicyclic nucleic acids (BNA); replacement of the ribosyl ring oxygen atom with S, N(R), or C(R¹)(R²) (R=H, C₁-C₁₂ alkyl or a protecting group); and combinations thereof. Examples of chemically modified sugars include, 2′-F-5′-methyl substituted nucleoside (see, PCT International Application WO 2008/101157, published on Aug. 21, 2008 for other disclosed 5′, 2′-bis substituted nucleosides), replacement of the ribosyl ring oxygen atom with S with further substitution at the 2′-position (see, published U.S. Patent Application US2005/0130923, published on Jun. 16, 2005), or, alternatively, 5′-substitution of a BNA (see, PCT International Application WO 2007/134181, published on Nov. 22, 2007, wherein LNA is substituted with, for example, a 5′-methyl or a 5′-vinyl group).

Examples of nucleosides having modified sugar moieties include, without limitation, nucleosides comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—C₁-C₁₀ alkyl, OCF₃, O(CH₂)₂SCH₃, O(CH₂)₂—O—N(R^{m})(Rⁿ), and O—CH₂—C(═O)—N(R^{m})(Rⁿ), where each R^{m} and Rⁿ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include, without limitation, nucleosides comprising a bridge between the 4′ and the 2′ ribosyl ring atoms. In certain embodiments, antisense oligonucleotides provided herein include one or more bicyclic nucleosides wherein the bridge comprises a 4′ to 2′ bicyclic nucleoside. Examples of such 4′ to 2′ bicyclic nucleosides, include, but are not limited to, one of the formulae: 4′-(CH₂)—O-2′ (LNA); 4′-(CH₂)—S-2; 4′—(CH₂)₂—O-2′ (ENA); 4′-CH(CH₃)—O-2′ (cEt) and 4′-CH(CH₂OCH₃)—O-2′, and analogs thereof (see, U.S. Pat. No. 7,399,845, issued on Jul. 15, 2008); 4′-C(CH₃)(CH₃)—O-2′, and analogs thereof (see, published PCT International Application WO2009/006478, published Jan. 8, 2009); 4′-CH₂—N(OCH₃)-2′, and analogs thereof (see, published PCT International Application WO2008/150729, published Dec. 11, 2008); 4′-CH₂—O—N(CH₃)-2′ (see, published U.S. Patent Application US2004/0171570, published Sep. 2, 2004); 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, Chattopadhyaya, et al., J. Org. Chem., 2009, 74, 118-134); and 4′-CH₂—C(═CH₂)-2′, and analogs thereof (see, published PCT International Application WO 2008/154401, published on Dec. 8, 2008). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 6,670,461, 7,053,207, 6,268,490, 6,770,748, 6,794,499, 7,034,133, 6,525,191, 7,399,845; published PCT International applications WO 2004/106356, WO 94/14226, WO 2005/021570, and WO 2007/134181; U.S. Patent Publication Nos. US2004/0171570, US2007/0287831, and US2008/0039618; and U.S. patent Ser. Nos. 12/129,154, 60/989,574, 61/026,995, 61/026,998, 61/056,564, 61/086,231, 61/097,787, and 61/099,844; and PCT International Application Nos. PCT/US2008/064591, PCT/US2008/066154, and PCT/US2008/068922. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, oligonucleotides having at least one bridge between the 4′ and the 2′ position of the pentofuranosyl sugar moiety wherein such bridges independently comprises 1 or from 2 to 4 linked groups independently selected from —[C(R^{a})(R^{b})]ₙ—, —C(R^{a})═C(R^{b})—, —C(R^{a})═N—, —C(═NR^{a})—, —C(═O)—, —C(═S)—, —O—, —Si(R^{a})₂—, —S(═O)ₓ—, and —N(R^{a})—;
wherein:
x is 0, 1, or 2;
n is 1, 2, 3, or 4;
each R^{a} and R^{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ¹, NJ¹J², SJ¹, N₃, COOJ¹, acyl (C(═O)—H), substituted acyl, CN, sulfonyl (S(═O)₂-J¹), or sulfoxyl (S(═O)-J¹); and
each J¹ and J² is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

In certain embodiments, the bridge of a bicyclic sugar moiety is, —[C(R^{a})(R^{b})]ₙ—, —[C(R^{a})(R^{b})]ₙ—O—, —C(R^{a})(R^{b})—N(R)—O— or, C(R^{a})(R^{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′, and 4′-CH₂—N(R)—O-2′-, wherein each R is, independently, H, a protecting group, or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNAs have been incorporated into antisense oligonucleotides that showed antisense activity (Frieden et al., Nucleic Acids Research, 2003, 21, 63 6563 72).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

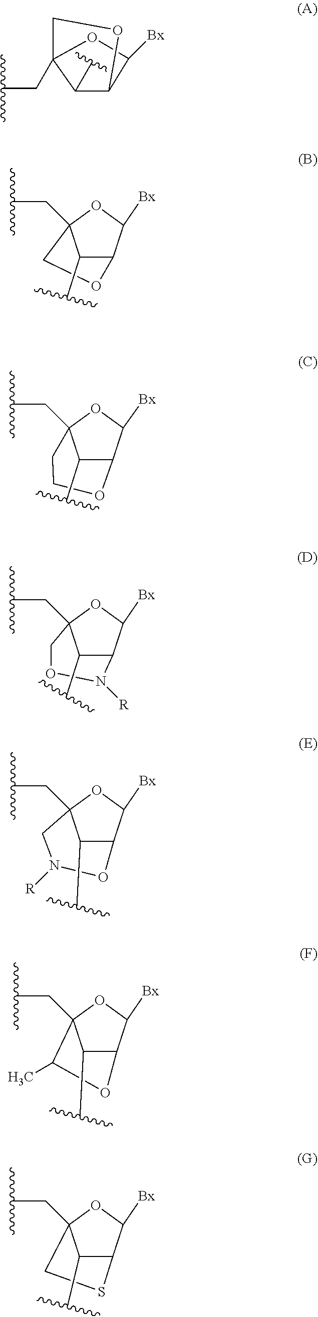

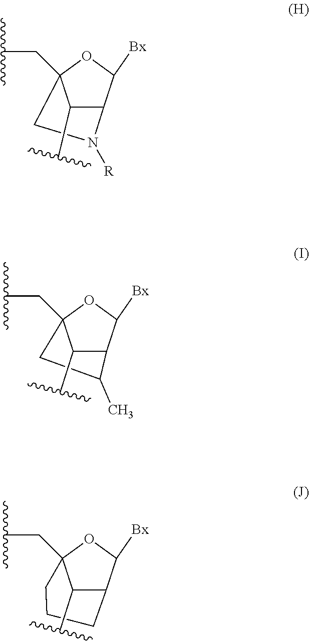

wherein Bx is the base moiety and R is, independently, H, a protecting group or C₁-C₁₂ alkyl.

In certain embodiments, the bicyclic nucleoside is according to Formula I:

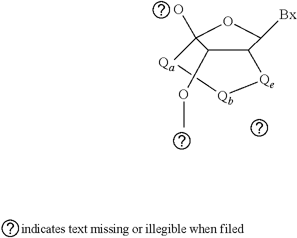

wherein:
Bx is a heterocyclic base moiety;
-Q^{a}-Q^{b}-Q^{c}- is —CH₂—N(R_{c})—CH₂—, —C(═O)—N(R^{c})—CH₂—, —CH₂—O—N(R^{c})—, —CH₂—N(R^{c})—O—, or —N(R^{c})—O—CH₂;
R^{c} is C₁-C₁₂ alkyl or an amino protecting group; and
T^{a} and T^{b} are each, independently, H, a hydroxyl protecting group, a conjugate group, a reactive phosphorus group, a phosphorus moiety, or a covalent attachment to a support medium.

In certain embodiments, bicyclic nucleoside having Formula II:

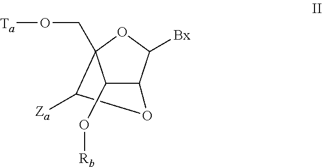

wherein:
Bx is a heterocyclic base moiety;
T^{a} and T^{b} are each, independently, H, a hydroxyl protecting group, a conjugate group, a reactive phosphorus group, a phosphorus moiety, or a covalent attachment to a support medium;
Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio.

In one embodiment, each of the substituted groups is, independently, mono or poly substituted with substituent groups independently selected from halogen, oxo, hydroxyl, OJ^{c}, NJ^{c}J^{d}, SJ^{c}, N₃, OC(═X)J^{c}, and NJ^{e}C(═X)NJ^{c}J^{d}, wherein each J^{c}, J^{d}, and J^{e} is, independently, H, C₁-C₆ alkyl, or substituted C₁-C₆ alkyl and X is O or NJ^{c}.

In certain embodiments, bicyclic nucleoside having Formula III:

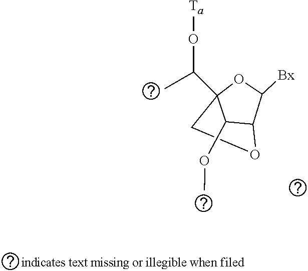

wherein:
Bx is a heterocyclic base moiety;
T^{a} and T^{b} are each, independently, H, a hydroxyl protecting group, a conjugate group, a reactive phosphorus group, a phosphorus moiety, or a covalent attachment to a support medium;
Z^{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, or substituted acyl (C(═O)—).

In certain embodiments, bicyclic nucleoside having Formula IV:

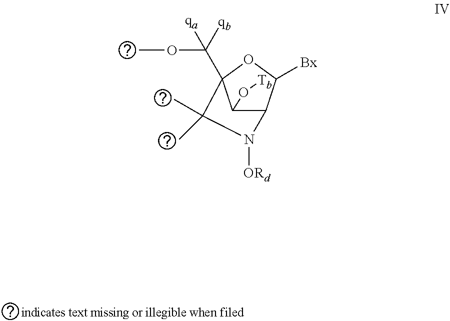

wherein:
Bx is a heterocyclic base moiety;
T^{a} and T^{b} are each, independently H, a hydroxyl protecting group, a conjugate group, a reactive phosphorus group, a phosphorus moiety, or a covalent attachment to a support medium;
R^{d} is C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl;
each q^{a} q^{b}, q^{c} and q^{d} is, independently, H, halogen, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl, C₁-C₆ alkoxyl, substituted C₁-C₆ alkoxyl, acyl, substituted acyl, C₁-C₆ aminoalkyl, or substituted C₁-C₆ aminoalkyl;

In certain embodiments, bicyclic nucleoside having Formula V:

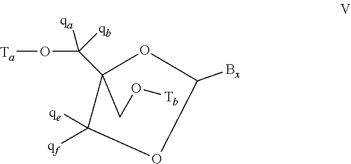

wherein:
Bx is a heterocyclic base moiety;
T^{a} and T^{b} are each, independently, H, a hydroxyl protecting group, a conjugate group, a reactive phosphorus group, a phosphorus moiety, or a covalent attachment to a support medium;
q^{a}, q^{b}, q^{e} and q^{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ^{j}, SJ^{j}, SOJ^{j}, SO₂J^{j}, NJ^{j}J^{k}, N₃, CN, C(═O)OJ^{j}, C(═O)NJ^{j}J^{k}, C(═O)J^{j}, O—C(═O)NJ^{j}J^{k}, N(H)C(═NH)NJ^{j}J^{k}, N(H)C(═O)NJ^{j}J^{k} or N(H)C(═S)NJ^{j}J^{k};
or q^{e} and q^{f} together are ═C(g^{g})(q^{h});
q⁹ and q^{h} are each, independently, H, halogen, C₁-C₁₂ alkyl, or substituted C₁-C₁₂ alkyl.

The synthesis and preparation of the methyleneoxy (4′-CH₂—O-2′) BNA monomers adenine, cytosine, guanine, 5-methyl-cytosine, thymine, and uracil, along with their oligomerization, and nucleic acid recognition properties have been described (see, e.g., Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA, and 2′-thio-BNAs, have also been prepared (see, e.g., Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (see, e.g., Wengel et al., WO 99/14226). Furthermore, synthesis of 2′-amino-BNA, a novel conformationally restricted high-affinity oligonucleotide analog, has been described in the art (see, e.g., Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

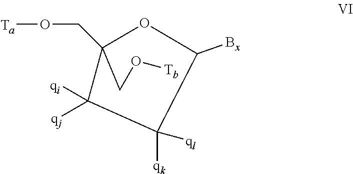

wherein:
Bx is a heterocyclic base moiety;
T^{a} and T^{b} are each, independently, H, a hydroxyl protecting group, a conjugate group, a reactive phosphorus group, a phosphorus moiety, or a covalent attachment to a support medium;
each qⁱ, q^{j}, q^{k} and q^{l} is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJ^{j}, SJ^{j}, SOJ^{j}, SO₂J^{j}, NJ^{j}J^{k}, N₃, CN, C(═O)OJ^{j}, C(═O)NJ^{j}J^{k}, C(═O)J^{j}, O—C(═O)NJ^{j}J^{k}, N(H)C(═NH)NJ^{j}J^{k}, N(H)C(═O)NJ^{j}J^{k}, or N(H)C(═S)NJ^{j}J^{k}; and
qⁱ and q^{j} or q^{l} and q^{k} together are ═C(q^{g})(q^{h}), wherein q^{g} and q^{h}, are each, independently, H, halogen, C₁-C¹² alkyl, or substituted C₁-C₁₂ alkyl.

One carbocyclic bicyclic nucleoside having a 4′-(CH₂)₃-2′ bridge and the alkenyl analog, bridge 4′-CH═CH—CH₂-2′, have been described (see, e.g., Freier et al., Nucleic Acids Research, 1997, 25(22), 44294443 and Albaek et al., J. Org. Chem., 2006, 71, 7731-7740). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

As used herein, “bicyclic nucleoside” refers to a nucleoside comprising a bridge connecting two carbon atoms of the sugar ring, thereby forming a bicyclic sugar moiety. In certain embodiments, the bridge connects the 2′ carbon and another carbon of the sugar ring.

As used herein, “4′-2′ bicyclic nucleoside” or “4′ to 2′ bicyclic nucleoside” refers to a bicyclic nucleoside comprising a furanose ring comprising a bridge connecting the 2′ carbon atom and the 4′ carbon atom.

As used herein, “monocyclic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl; substituted alkyl; alkenyl; alkynyl; alkaryl; aralkyl; O-alkaryl or O-aralkyl; SH; SCH₃; OCN; Cl; Br; CN; CF₃; OCF₃; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving pharmacokinetic properties; and a group for improving the pharmacodynamic properties of an antisense oligonucleotide, and other substituents having similar properties. In certain embodiments, modified nucleosides comprise a 2′-MOE side chain (see, e.g., Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (see, e.g., Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), mannitol nucleic acid (MNA) (see Leumann, C J. Bioorg. & Med. Chem. (2002) 10:841-854), fluoro HNA (F-HNA), or those oligonucleotides having Formula X:
wherein independently for each of said at least one tetrahydropyran nucleoside analog of Formula X:

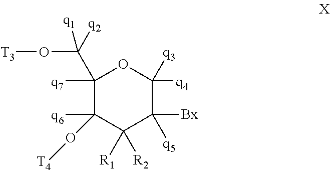

Bx is a heterocyclic base moiety;
T³ and T⁴ are each, independently, an internucleoside linking group linking the tetrahydropyran nucleoside analog to the antisense oligonucleotide or one of T³ and T⁴ is an internucleoside linking group linking the tetrahydropyran nucleoside analog to the antisense oligonucleotide and the other of T³ and T⁴ is H, a hydroxyl protecting group, a linked conjugate group, or a 5′ or 3′-terminal group;
q¹, q², q³, q⁴, q⁵, q⁶, and q⁷ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
one of R¹ and R² is hydrogen and the other is selected from halogen, substituted or unsubstituted alkoxy, NJ¹J², SJ¹, N₃, OC(═X)J¹, OC(═X)NJ¹J², NJ³C(═X)NJ¹J², and CN, wherein X is O, S, or NJ¹, and each J¹, J², and J³ is, independently, H or C₁-C₆ alkyl.

In certain embodiments, the modified THP nucleosides of Formula X are provided wherein q^{m}, qⁿ, q^{p}, q^{r}, q^{s}, q^{t} and q^{u} are each H. In certain embodiments, at least one of q^{m}, qⁿ, q^{p}, q^{r}, q^{s}, q^{t}, and q^{u} is other than H. In certain embodiments, at least one of q^{m} qⁿ, q^{p}, q^{r}, q^{s}, q^{t}, and q^{u} is methyl. In certain embodiments, THP nucleosides of Formula X are provided wherein one of R¹ and R² is F. In certain embodiments, R¹ is fluoro and R² is H, R¹ is methoxy and R² is H, and R¹ is methoxyethoxy and R² is H.

As used herein, “2′-modified nucleoside” or “2′-substituted nucleoside” refers to a nucleoside comprising a sugar comprising a substituent at the 2′ position of a furanose ring other than H or OH. 2′-modified nucleosides, include, but are not limited to, bicyclic nucleosides wherein the bridge connecting two carbon atoms of the sugar ring connects the 2′ carbon and another carbon of the sugar ring and nucleosides with non-bridging 2′-substituents, such as allyl, amino, azido, thio, O-allyl, O—C₁-C₁₀ alkyl, —OCF₃, O—(CH₂)₂—OCH₃, 2′-O(CH₂)₂SCH₃, O—(CH₂)₂—O—N(R^{m})(Rⁿ), or O—CH₂—C(═O)—N(R^{m})(Rⁿ), where each R^{m} and Rⁿ is, independently, H or substituted or unsubstituted C₁-C₁₀ alkyl. 2′-modified nucleosides may further comprise other modifications, for example, at other positions of the sugar and/or at the nucleobase.

As used herein, “2′-F” refers to a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense oligonucleotides (see, e.g., review article: Leumann, J. C, Bioorganic & Medicinal Chemistry, 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified, or a combination thereof) are maintained for hybridization with an appropriate nucleic acid target.

In certain embodiments, antisense oligonucleotides comprise one or more nucleotides having modified sugar moieties. In certain embodiments, the modified sugar moiety is 2′-MOE. In certain embodiments, the 2′-MOE modified nucleotides are arranged in a gapmer motif or a multi-segmented antisense oligonucleotide motif. In certain embodiments, the modified sugar moiety is a cEt. In certain embodiments, the cEt modified nucleotides are arranged throughout the wings of a gapmer motif or a multi-segmented antisense oligonucleotide motif.

### Compositions and Methods for Formulating Pharmaceutical Compositions

The oligonucleotides of the disclosure may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Pharmaceutical compositions comprising antisense oligonucleotides encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligonucleotides, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

The disclosure provides pharmaceutical compositions comprising the modified multi-segmented antisense oligonucleotides of the disclosure and a pharmaceutically acceptable carrier, diluent or excipient. In some embodiments, the modified multi-segmented antisense oligonucleotide comprises a structure of the sequence of any one of the oligonucleotide sequences described herein.

Modified multi-segmented antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

The pharmaceutical compositions of the disclosure can optionally comprise therapeutic agents, pharmaceutical agents, carriers, adjuvants, dispersing agents, diluents, and the like.

Pharmaceutical compositions comprising modified multi-segmented antisense oligonucleotides can encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligonucleotides, prodrugs, pharmaceutically acceptable salts of such prodrugs, and other bioequivalents. Suitable pharmaceutically acceptable salts include, but are not limited to, sodium and potassium salts.

Modified multi-segmented antisense oligonucleotides formulated as prodrugs are contemplated as within the scope of the instant disclosure. A prodrug can include the incorporation of additional nucleosides at one or both ends of an antisense oligonucleotide which are cleaved by endogenous nucleases within the body, to form the active antisense oligonucleotide.

Pharmaceutical compositions can contain any of the reagents discussed above, and one or more of a pharmaceutically acceptable carrier, a diluent or an excipient.

A “pharmaceutical composition” is a formulation comprising the modified multi-segmented antisense oligonucleotides described herein, in a form suitable for administration to a subject. In certain embodiments, the pharmaceutical composition is in bulk or in unit dosage form. The unit dosage form is any of a variety of forms, including, for example, a capsule, an IV bag, a tablet, a single use injector, a single pump on an aerosol inhaler or a vial. The quantity of active ingredient (e.g., a formulation of the modified multi-segmented antisense oligonucleotide) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a of this invention include powders, sprays, ointments, pastes, creams, lotions, gels, solutions, patches and inhalants. In certain embodiments, the modified multi-segmented antisense oligonucleotide is mixed under sterile conditions with a pharmaceutically acceptable carrier, and with any preservatives, buffers, or propellants that are required.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification includes both one and more than one such excipient.

A pharmaceutical composition is formulated to be compatible with its intended route of administration. Examples of routes of administration include parenteral, e.g., intravenous, intradermal, subcutaneous, oral (e.g., inhalation), transdermal (topical), intraperitoneal (into the body cavity) and transmucosal administration. Solutions or suspensions used for parenteral, intradermal, intraperitoneal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral or subcutaneous preparation can be enclosed in ampoules, disposable syringes or multiple dose vials made of glass or plastic. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules, syringes and vials, and can be stored in a freeze-dried (lyophilized) condition requiring only the addition of the sterile liquid carrier, for example, saline or water-for-injection immediately prior to use.

The pharmaceutical composition described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active agents into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible pharmaceutically acceptable carrier. They can be enclosed in gelatin capsules or compressed into tablets. For the purpose of oral therapeutic administration, the active age can be incorporated with excipients and used in the form of tablets, troches, or capsules. Oral compositions can also be prepared using a fluid carrier for use as a mouthwash, wherein the agents in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the agents are delivered in the form of an aerosol spray from pressured container or dispenser, which contains a suitable propellant, e.g., a gas such as carbon dioxide, or a nebulizer.

Pharmaceutical compositions can be prepared with pharmaceutically acceptable carriers that will protect the modified multi-segmented antisense oligonucleotides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, and the materials can be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of the modified multi-segmented antisense oligonucleotide calculated to produce the desired therapeutic effect in association with the required pharmaceutical carrier. The specification for the dosage unit forms of the disclosure are dictated by and directly dependent on the unique characteristics of the active agent and the particular therapeutic effect to be achieved.

The pharmaceutical compositions can be included in a container, pack, or dispenser together with instructions for administration.

Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids.

Techniques for formulation and administration of the disclosed compositions of the invention can be found in Remington: the Science and Practice of Pharmacy, 19th edition, Mack Publishing Co., Easton, PA (1995).

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention. Modified multi-segmented antisense oligonucleotides of the disclosure can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Modified multi-segmented antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense oligonucleotide to impart nuclease stability include those disclosed in WO 03/004602 published on Jan. 16, 2003.

Modified multi-segmented antisense oligonucleotides of the disclosure can be encapsulated within, or incorporated on the surface of particles. In certain embodiments, the particle is a nanoparticle. Exemplary nanoparticles include liposomes, micelles, polymer-based nanoparticles, lipid-polymer based nanoparticles, and polymeric micelles.

In certain embodiments, the nanoparticle comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published Application 20090023673. In other embodiments, the one or more lipids are one or more cationic lipids. One skilled in the art will recognize which liposomes are appropriate for encapsulation of the modified multi-segmented antisense oligonucleotides described herein.

In certain embodiments, the nanoparticle comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In certain embodiments, the nanoparticle comprises a polymer based nanoparticle. Polymer based nanoparticles comprise one or more polymers, such as a polyester, poly(orthoester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In exemplary embodiments, the one or more polymers comprise polyalkylene glycol such as polyethylene glycol (PEG), or polyalkylene oxide, such as polyethylene oxide (PEO).

In some embodiments, the nanoparticles or some portion thereof are degradable. In other embodiments, the lipids and/or polymers of the nanoparticles are degradable.

### Conjugated Antisense Oligonucleotides

Antisense oligonucleotides may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense oligonucleotide to impart nuclease stability include those disclosed in WO 03/004602 published on Jan. 16, 2003.

### Cell Culture and Antisense Oligonucleotides Treatment

The effects of antisense oligonucleotides on the level, activity or expression of target nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and are cultured according to the vendor's instructions using commercially available reagents (e.g. Invitrogen Life Technologies, Carlsbad, CA). Illustrative cell types include, but are not limited to, HuVEC cells, b.END cells, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense oligonucleotides.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides may be mixed with LIPOFECTIN in OPTI-MEM 1 (Invitrogen, Carlsbad, CA) to achieve the desired final concentration of antisense oligonucleotide and a LIPOFECTIN concentration that may range from 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 ug/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Analysis of Inhibition of Target Levels or Expression

Inhibition of levels or expression of a target nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, CA). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, CA). Cyclophilin A expression is quantified by real time PCR, by being run simultaneously with the target, multiplexing, or separately. Total RNA is quantified using RIBOGREEN RNA quantification reagent (Invitrogen, Inc. Eugene, OR). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a target nucleic acid. Methods for designing real-time PCR probes and primers are well known in the art, and may include the use of software such as PRIMER EXPRESS Software (Applied Biosystems, Foster City, CA).

### Quantitative Real-Time PCR Analysis of Target DNA Levels

Quantitation of target DNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Gene (or DNA) target quantities obtained by real time PCR are normalized using either the expression level of a gene whose expression is constant, such as cyclophilin A, or by quantifying total DNA using RIBOGREEN (Invitrogen, Inc. Carlsbad, CA). Cyclophilin A expression is quantified by real time PCR, by being run simultaneously with the target, multiplexing, or separately. Total DNA is quantified using RIBOGREEN RNA quantification reagent (Invetrogen, Inc. Eugene, OR). Methods of DNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a target nucleic acid. Methods for designing real-time PCR probes and primers are well known in the art, and may include the use of software such as PRIMER EXPRESS Software (Applied Biosystems, Foster City, CA).

### Analysis of Protein Levels

Antisense inhibition of target nucleic acids can be assessed by measuring target protein levels. Protein levels of target can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Oligonucleotides

Antisense oligonucleotides, for example, antisense oligonucleotides, are tested in animals to assess their ability to inhibit expression of target and produce phenotypic changes. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, subcutaneous, intrathecal, and intracerebroventricular. Administration also includes intra-ocular and intramuscular routes of administration. Calculation of antisense oligonucleotide dosage and dosing frequency is within the abilities of those skilled in the art, and depends upon factors such as route of administration and animal body weight.

### Certain Combination Therapies

In certain embodiments, one or more pharmaceutical compositions provided herein are co-administered with one or more other pharmaceutical agents. In certain embodiments, such one or more other pharmaceutical agents are designed to treat the same disease, disorder, or condition as the one or more pharmaceutical compositions provided herein. In certain embodiments, such one or more other pharmaceutical agents are designed to treat a different disease, disorder, or condition as the one or more pharmaceutical compositions provided herein.

In certain embodiments, such one or more other pharmaceutical agents are designed to treat an undesired side effect of one or more pharmaceutical compositions provided herein. In certain embodiments, one or more pharmaceutical compositions provided herein are co-administered with another pharmaceutical agent to treat an undesired effect of that other pharmaceutical agent. In certain embodiments, one or more pharmaceutical compositions provided herein are co-administered with another pharmaceutical agent to produce a combinational effect. In certain embodiments, one or more pharmaceutical compositions provided herein are co-administered with another pharmaceutical agent to produce a synergistic effect.

In certain embodiments, one or more pharmaceutical compositions provided herein and one or more other pharmaceutical agents are administered at the same time. In certain embodiments, one or more pharmaceutical compositions provided herein and one or more other pharmaceutical agents are administered at different times. In certain embodiments, one or more pharmaceutical compositions provided herein and one or more other pharmaceutical agents are prepared together in a single formulation. In certain embodiments, one or more pharmaceutical compositions provided herein and one or more other pharmaceutical agents are prepared separately.

While certain oligonucleotides, compositions and methods described herein have been described with specificity in accordance with certain embodiments, the following examples serve only to illustrate the oligonucleotides described herein and are not intended to limit the same.

### EXAMPLES

### Example 1: Synthesis and Assessment of Modified Multi-Segmented Antisense Oligonucleotides

The following example describes experimental methods used to synthesize modified oligonucleotides and assess in vitro inhibition of target nucleotides.

### Modified Oligonucleotide Sequences and Methods of Making

Exemplary modified multi-segmented antisense oligonucleotides are shown in Table 1A, Table 1B, Table 1C and FIG. 22. Specifically, Table 1A shows modified multi-segmented antisense oligonucleotides for inhibiting HBV. Table 1B shows modified multi-segmented antisense oligonucleotides for inhibiting PCSK9, DMPK, TTR, or AAT. Table 2.2 shows a legend describing the individual modifications at each position of the modified multi-segmented antisense oligonucleotides in Table 1A and Table 1B.

Table 2.1, Table 1C and FIG. 22 show further exemplary modified multi-segmented antisense oligonucleotides. Table 2.2 shows a legend describing the individual modifications at each position of the modified multi-segmented antisense oligonucleotides in Table 2.1. In Table 1C and FIG. 22, lowercase font represents a 2′-deoxynucleoside, and uppercase font represents a nucleoside with any one of the modifications shown in the section titled Exemplary Modifications, such as a 2′-O-methoxyethyl nucleoside, 2′-O-methoxyethyl cytidine, 2′-O-methyl nucleoside, 2′-O-methyl nucleoside, 2′-OH nucleoside, GNA, LNA, LNA with cytosine base, 2′-Fluoro 2′-deoxy nucleoside, 2′-Fluoro 2′-deoxy cytidine, 2′-F-Arabinonucleic Acid (2′-F-ANA), or 2′-F-Arabinonucleic Acid (2′-F-ANA), or a combination thereof. In Table 1C and FIG. 22, lowercase “c” represents a 2′-deoxy cytidine or 2′-deoxy 5-methylcytidine, and upper case “C” represents a cytidine or 5-methylcytidine with or without the above described sugar modifications.

Modified multi-segmented antisense oligonucleotides were generated with various structural motifs. For example one motif included, from 5′ to 3′, a 5′ wing that is 4 nucleobases in length, a first gap that is 5 nucleobases in length, a separator that is 1 nucleobase in length, a second gap 5 nucleobases in length, and a 3′ wing that is 5 nucleobases in length, together referred to herein as a “4-5-1-5-5” motif. In Table 1A, Table 1B, and Table 1C, the separator is shown in bolded text.

Synthesis: Solid-phase synthesis is used to produce a modified multi-segmented antisense oligonucleotide. Oligonucleotides are tethered to a solid surface when they are being made. In other words, within the solid-phase synthesis, an oligonucleotide being assembled is covalently bound, via its 3′-terminal hydroxyl group, to solid support material and remains attached to it over the entire course of the chain assembly. This synthesis process begins with the 3′-most nucleotide and proceeds through a series of cycles composed of four steps that are repeated until the 5′-most nucleotide is attached. These four steps are deprotection (A), condensation (B), capping (C), and oxidation (D), as stated below:
Step 1: Remove the DMTr attached to the 5′-OH group of the first base on the CPG monomer, and prepare to attach the next new base;
Step 2: Activate the new base monomer and prepare to react with the first base;
Step 3: The second base has a coupling reaction with the first base;
Step 4: Cap the 5′-OH of the first base that has not reacted so that it will no longer participate in the reaction;
Step 5: Oxidation of nucleoside phosphites to more stable nucleoside phosphates;
Step 6: Repeat the cycle of Step1~Step5 until the required oligonucleotide DNA sequence is synthesized.

Aminolysis: The synthesized modified multi-segmented antisense oligonucleotide is chemically cleaved from the solid carrier (CPG), and the protective group is removed by aminolysis.

Purification: The modified primer purification method is as follows:
(1) PAGE purification: use denaturing polyacrylamide gel electrophoresis to separate the primer DNA then the target DNA is recovered from the gel. The purity of the purified DNA is greater than 90%.
(2) HPLC purification: use the principle of high performance liquid chromatography to purify the primer DNA to a purity greater than 90%.

### Antisense Inhibition of HBV in HepG2.2.15 Cells

The antiviral effect of modified multi-segmented antisense oligonucleotides targeting HBV was evaluated by measuring the levels of hepatitis B virus (HBV) surface antigen (HBsAg) and HBV e antigen (HBeAg) in HepG2.2.15 cells. The HBsAg levels were measured by chemiluminescence immunoassays, the HBeAg levels were measured by enzyme-linked immunosorbent assay (ELISA) assays, and cell viability was determined by MTT cell proliferation assay. The calculation result was expressed as mean and SD.

HepG2.2.15 cells that can stably express HBsAg and HBeAg were purchased from China Center for Type Culture Collection (CCTCC No. GDC0141). The modified multi-segmented antisense oligonucleotide solutions were prepared by dissolving each compound in saline or RNase-free double distilled H₂O (dd H₂O) to make a target stock solution at 50 μM.

The modified multi-segmented antisense oligonucleotides were set in a series of concentrations (0, 0.037, 0.11, 0.34, 1, 3.1, 9.3, 27.8, 83.3, 250 nM) and worked with HepG2.2.15 cells by reverse transfection. First, the 96-well cell culture plate was covered with 50 ul Rat Tail Collagen I in every well and removed one hour later. Second, P3000 and Lipofectamine® 3000 Reagent was diluted with Opti-MEM™ reduced serum medium and mixed with the serial dilution compound solution, followed by incubation at room temperature for 10 minutes. Third, 10 ul of transfection reagent mixture of P3000, Lipo3000 and modified multi-segmented antisense oligonucleotide, and 100 ul of cell suspension with a concentration of 3.5*10⁵ cells were added to each well.

The mixture was pipetted 4 or 5 times and the plate was placed in a 37° C. incubator. After reverse transfection 24 hour, the media was exchanged with fresh cell culture medium. Following a 48 hour culture, supernatant was collected for detection. The remaining cells were used for cell viability test. The resulting data is represented in Table 1A, as EC50 (nM) and MTT CC25 (nM). The EC50 (nM) ratio was calculated by dividing the EC50 value determined following the treatment with the modified multi-segmented antisense oligonucleotide shown in Table 1A by the EC50 value determined following the treatment with a reference oligonucleotide of AUS1233 (SEQ ID NO: 10), a “gapmer” oligonucleotide that lacks a separator sequence. A lower EC50 ratio value suggests the increased ability of the modified multi-segmented antisense oligonucleotide to decrease HBsAg levels relative to the reference oligonucleotide. The MTT CC25 (nM) ratio was calculated by dividing the MTT CC25 value determined following treatment with the modified multi-segmented antisense oligonucleotide shown in Table 1A by the MTT CC25 value determined following the treatment with a reference oligonucleotide of AUS1233 (SEQ ID NO: 10). A higher MTT CC25 ratio suggests an increased cellular viability relative to the reference oligonucleotide. The C/E ratio was calculated by dividing the MTT CC25 ratio value by the EC50 value. A higher C/E ratio suggests a balanced decrease in HBsAg levels while maintaining high cellular viability.

### Antisense Inhibition of PCSK9, DMPK, AAT, and TTR

The effects of modified multi-segmented antisense oligonucleotides targeting PCSK9, DMPK, AAT, or TTR were evaluated as described below. The resulting data is represented in Table 1B, as EC25 (nM), EC50 (nM) and the maximum inhibition (%).

### Stability of Modified Multi-Segmented Antisense Oligonucleotides

Table 1A and Table 1B also provide melting temperature data for representative modified multi-segmented antisense oligonucleotides, as an assessment of their stability, as described in Example 11, below.

### Example 7: Inhibition of Murine PCSK9 Expression in Hepa1-6 Cells Using Modified Multi-Segmented Antisense Oligonucleotides

Modified multi-segmented antisense oligonucleotide that are 20 nucleosides in length were designed to target a PCSK9 nucleic acid and were tested for their effects on PCSK9 mRNA in vitro.

The modified multi-segmented antisense oligonucleotides were tested in a series of experiments that had similar culture conditions. The results for each experiment are presented in separate tables shown below

Cultured Hepa1-6 cells at a density of 18,000 cells per well were transfected using Lipofectamine 3000 reagent according to the manufacturer's protocol. Reverse transfection of cells on ASO is a powerful alternative method to perform high-throughput transfections.

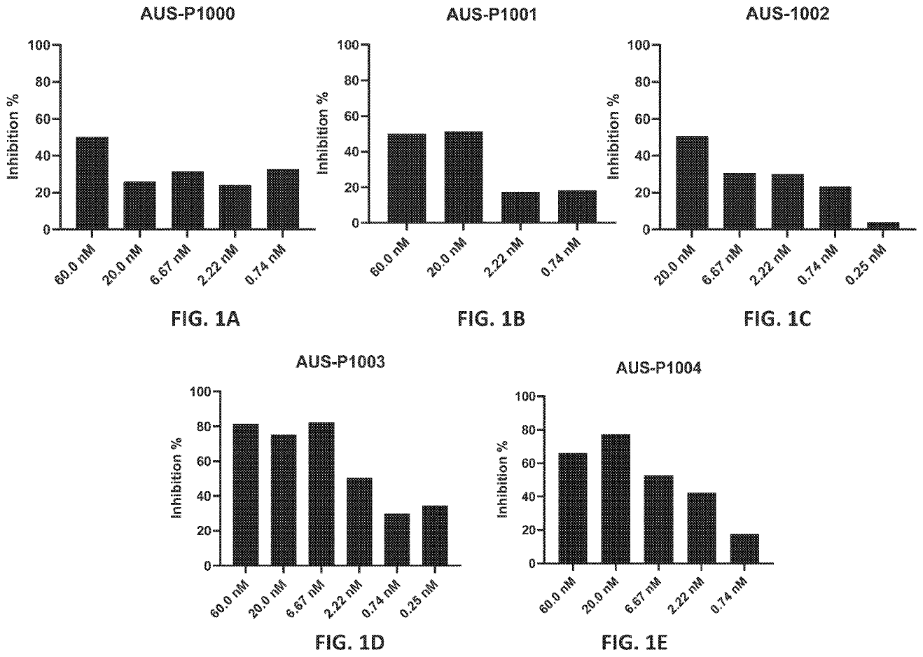

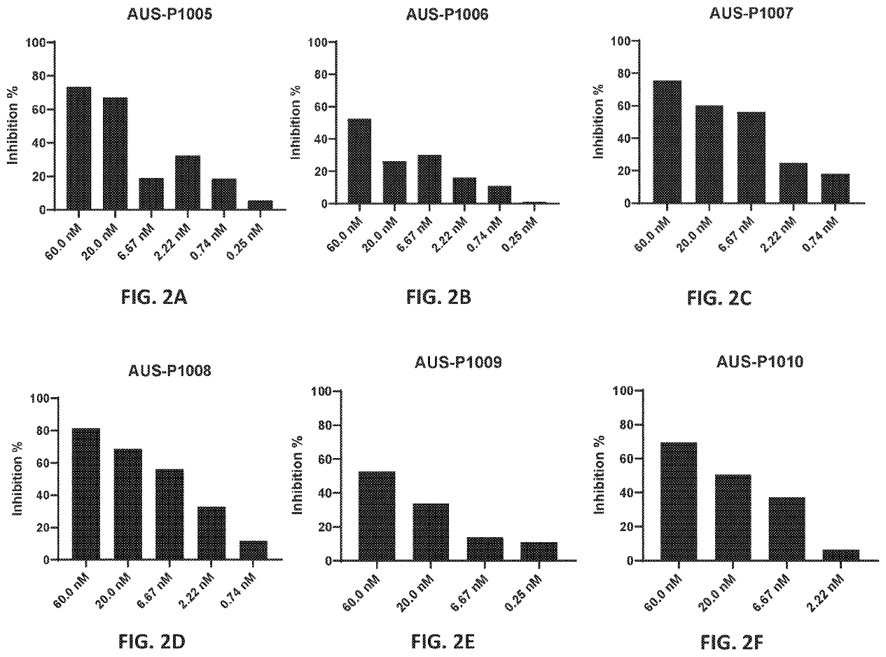

The cells were treated with a series dilution from 60 nM. After a treatment period of approximately 48 hours, RNA was isolated from the cells and PCSK9 mRNA levels were measured by quantitative real-time PCR. PCSK9 mRNA levels were measured using a mouse PCSK9 primer probe and normalized to housekeeping gene GAPDH mRNA levels. Results are presented as percent inhibition of PCSK9, relative to untreated control cells. ISIS 394816 (AB-1648) as described in U.S. Pat. No. 9,650,636 B2 and WO2009148605A2 was tested as a s control. Several of the newly designed modified multi-segmented antisense oligonucleotides were more potent than the reference control ISIS 394816 (AUS-P1000) (Table 7A, FIGS. 1A-1E and FIGS. 2A-2F). Table 2.2 shows a legend for the modifications at each of the residues shown in Table 7A.

A separator placed at position 10 of a modified multi-segmented antisense oligonucleotide that is 20 residues in length resulted in greater maximum inhibition relative to the control gapmer sequence that did not have a separator. Further, LNA modifications in the 3′ wing also increased the maximum inhibition relative to the control gapmer sequence that did not have modifications at the 3′ wing. The combination of a separator at position 10 and LNA modifications at position 17 and 18 (SEQ ID NO: 671 and SEQ ID NO: 675) resulted in the greatest maximum inhibition.

TABLE 7A
Antisense inhibition of PCSK9 in murine cells

| | SEQ | | | | | Maximum |
|---|---|---|---|---|---|---|
| | ID | | | EC₂₅ | EC₅₀ | Inhibition |
| AUS No. | NO: | Sequence | Motif* | (nM) | (nM) | % |
| AUS- | 667 | GGGCTcatagcacatTATCC | 5-10-5 | 4.51 | 80.1 | 32 |
| P1000 | | | | | | |
| AUS- | 668 | GGGCtcataGcacatTATCC | 4-5-1-5-5 | 0.94 | 258.1 | 59 |
| P1001 | | | | | | |
| AUS- | 669 | GGGCTcataGcacatTATCC | 5-4-1-5-5 | 6.19 | 33.9 | 51 |
| P1002 | | | | | | |
| AUS- | 670 | GGGCtcataGcacatT+A+TCC | 4-5-1-5-5 | 2.45 | / | 50 |
| P1003 | | | | | | |
| AUS- | 671 | GGGCTcataGcacatT+A+TCC | 5-4-1-5-5 | 0.36 | 1.92 | 82 |
| P1004 | | | | | | |
| AUS- | 672 | GGGCTcatagCacatTATCC | 5-5-1-4-5 | 1.27 | 6.17 | 77 |
| P1005 | | | | | | |
| AUS- | 673 | GGGCtcataGcacatT+AT+CC | 4-5-1-5-5 | 2.62 | 13.3 | 66 |
| P1006 | | | | | | |
| AUS- | 674 | GGGCtcataGcacatT+ATC+C | 4-5-1-5-5 | 8.5 | 59.02 | 30.1 |
| P1007 | | | | | | |
| AUS- | 675 | GGGCTcatagCacatT+A+TCC | 5-5-1-4-5 | 1.836 | 8.62 | 85.3 |
| P1008 | | | | | | |
| AUS- | 676 | GGGCTcataGcacatT+ATC+C | 5-4-1-5-5 | 1.678 | 6.46 | 68 |
| P1009 | | | | | | |
| AUS- | 677 | GGGCTcatagCacatT+ATC+C | 5-4-1-5-5 | 17.3 | 47.9 | 33 |
| P1010 | | | | | | |

*Motifs show the regions of each sequence from 5′ to 3′ (e.g., W1-G1-W2 or W1-G1-S1-G2-W2), where the separator sequence is shown in a bolded font.

### Example 8: Inhibition of Murine DMPK Expression in Sol8 Cells Using Modified Multi-Segmented Antisense Oligonucleotides

Modified multi-segmented antisense oligonucleotides targeted to a DMPK nucleic acid were tested for their effects on DMPK mRNA in vitro. Cultured murine skeletal muscle Sol8 cells at a density of 18,000 cells per well in a 96-well plate were treated with a series dilution from 60 nM of modified multi-segmented antisense oligonucleotide.

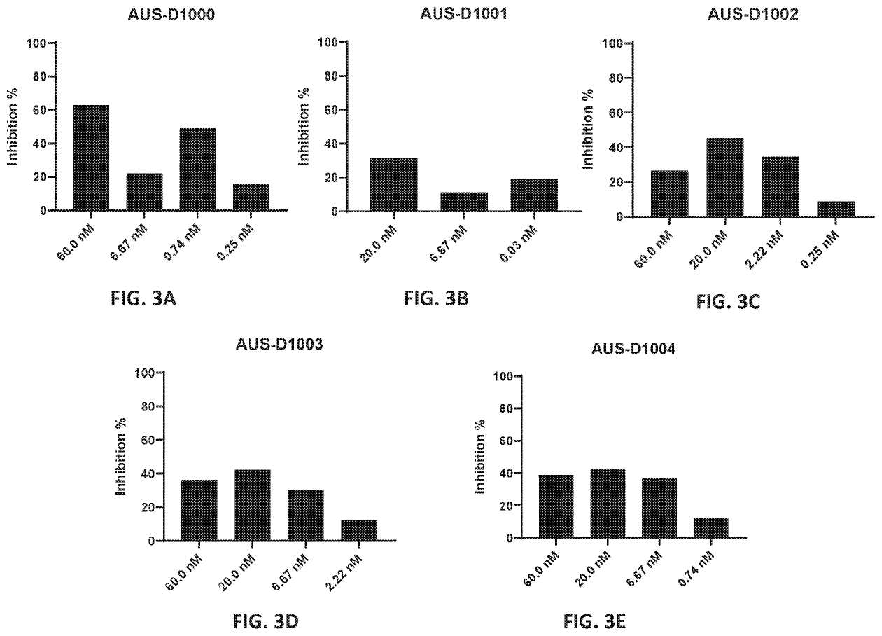

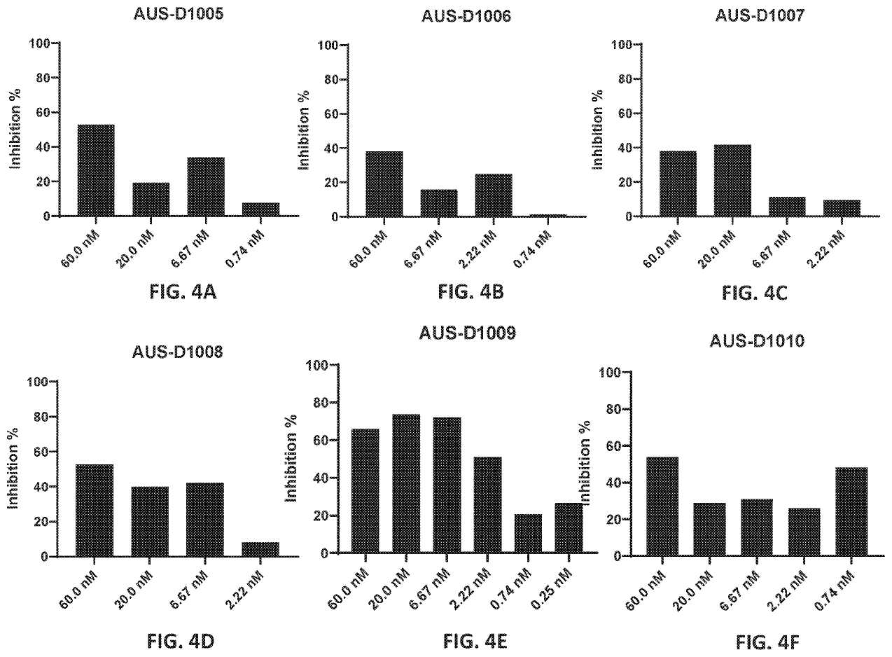

After a treatment period of approximately 48 hours, RNA was isolated from the cells and DMPK mRNA levels were measured by quantitative real-time PCR, as described herein. DMPK mRNA levels were measured using a mouse DMPK primer probe and normalized to housekeeping gene GAPDH mRNA levels. Results are presented as percent inhibition of DMPK, relative to untreated control cells. ISIS 486178 (AUS-DI000) as described in WO 2015/021457 A2 was tested as a reference control. Several of the novel modified multi-segmented antisense oligonucleotides were more potent than ISIS 486178 (AUS-D1000) (Table 8A, FIGS. 3A-3E and FIGS. 4A-4F). Table 2.2 shows a legend for the modifications at each of the residues shown in Table 8A.

A separator placed at position 10 of a modified multi-segmented antisense oligonucleotide that is 20 residues in length resulted in greater maximum inhibition relative to the control gapmer sequence that did not have a separator. Further, LNA modifications in the 3′ wing also increased the maximum inhibition relative to the control gapmer sequence that did not have modifications at the 3′ wing. The combination of a separator at position 10 and LNA modifications at position 17 and 20 (SEQ ID NO: 687) resulted in the greatest maximum inhibition.

TABLE 8A
Antisense inhibition of DMPK in murine cells

| | SEQ | | | | | Maximum |
|---|---|---|---|---|---|---|
| | ID | | | EC₂₅ | EC₅₀ | Inhibition |
| AUS No. | NO: | Sequence | Motif* | (nM) | (nM) | % |
| AUS- | 678 | ACAGAcaataaatacCGAGG | 5-10-5 | 40.0 | 96.4 | 21.3 |
| D1000 | | | | | | |
| AUS- | 679 | ACAGacaatAaatacCGAGG | 4-5-1-5-5 | 32.8 | / | 31.6 |
| D1001 | | | | | | |
| AUS- | 680 | ACAGAcaatAaatacCGAGG | 5-4-1-5-5 | 16.2 | 30.9 | 45.4 |
| D1002 | | | | | | |
| AUS- | 681 | ACAGacaatAaatacC+G+AGG | 4-5-1-5-5 | 45.3 | / | 42.3 |
| D1003 | | | | | | |
| AUS- | 682 | ACAGAcaatAaatacC+G+AGG | 5-4-1-5-5 | 6.5 | 101.9 | 42.5 |
| D1004 | | | | | | |
| AUS- | 683 | ACAGacaataaatacCGAGG | 4-11-5 | 12.6 | 60.0 | 34.0 |
| D1005 | | | | | | |
| AUS- | 684 | ACAGacaataAatacCGAGG | 4-6-1-4-5 | 34.6 | 160.2 | 24.8 |
| D1006 | | | | | | |
| AUS- | 685 | ACAGAcaataAatacCGAGG | 5-5-1-4-5 | 17.8 | 70.2 | 41.7 |
| D1007 | | | | | | |
| AUS- | 686 | ACAGacaatAaatacC+GA+GG | 4-5-1-5-5 | 6.7 | 36.0 | 42.2 |
| D1008 | | | | | | |
| AUS- | 687 | ACAGacaatAaatacC+GAG+G | 4-5-1-5-5 | 0.5 | 3.4 | 73.6 |
| D1009 | | | | | | |
| AUS- | 688 | ACAGAcaataAatacC+G+AGG | 5-5-1-4-5 | 2.7 | 58.8 | 48.1 |
| D1010 | | | | | | |

*Motifs show the regions of each sequence from 5′ to 3′ (e.g., W1-G1-W2 or W1-G1-S1-G2-W2), where the separator sequence is shown in a bolded font.

### Example 9: Inhibition of Murine TTR Expression in AML12 Cells Using Modified Multi-Segmented Antisense Oligonucleotides

Modified multi-segmented antisense oligonucleotides targeted to an TTR nucleic acid were tested for their effects on TTR mRNA in vitro. Cultured murine hepatocyte cells AML12 at a density of 18,000 cells per well in a 96-well plate were treated with a series dilution from 60 nM of modified multi-segmented antisense oligonucleotide.

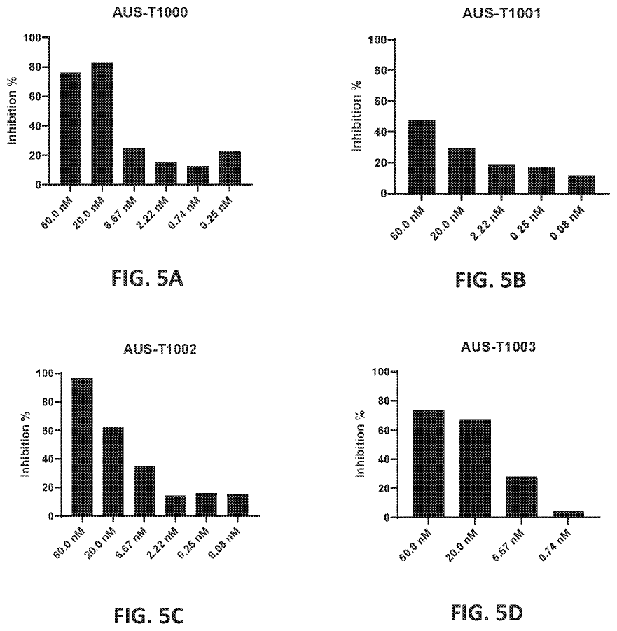

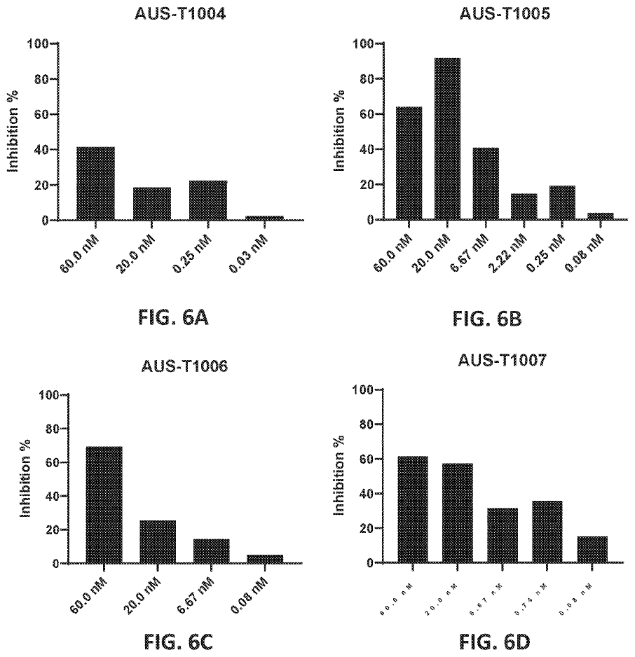

The sequence of AUS-T1001, AUS-T1002 and AUS-T1003 is the same as reference antisense nucleotide, but with distinctive structure in the gap length, nucleotide chemical modification and positioning of the modified nucleotides (Table 9A). AUS-T1004, AUS-T1005, AUS-T1006 and AUS-T1007 is newly designed with different target sites but has a common 5-10-5 gap-mer structure (Table 9B). After a treatment period of approximately 48 hours, RNA was isolated from the cells and TTR mRNA levels were measured by quantitative real-time PCR, as described herein. TTR mRNA levels were measured using a mouse TTR primer probe and normalized to housekeeping gene GAPDH mRNA levels. Results are presented as percent inhibition of TTR, relative to untreated control cells. ISIS 401724 (AUS-T1000) as previously described in U.S. Pat. No. 8,541,388 B2, was tested as a reference control. Several of the newly designed modified multi-segmented antisense oligonucleotides were more potent ISIS 401724 (AUS-T1000) (Table 9A and 9B, FIGS. 5A-5D and FIGS. 6A-6D). Table 2.2 shows a legend for the modifications at each of the residues shown in Table 9A.

A separator placed at position 10 of a modified multi-segmented antisense oligonucleotide that is 20 residues in length resulted in greater maximum inhibition relative to the control gapmer sequence that did not have a separator. Further, LNA modifications in the 3′ wing also increased the maximum inhibition relative to the control gapmer sequence that did not have modifications at the 3′ wing. The combination of a separator at position 10 and LNA modifications at position 17 and 20 (SEQ ID NO: 687) resulted in the greatest maximum inhibition.

TABLE 9A
Antisense inhibition of TTR in mouse

| | SEQ | | | | | Maximum |
|---|---|---|---|---|---|---|
| | ID | | | E_{C25} | EC₅₀ | Inhibition |
| AUS No. | NO: | Sequence | Motif* | (nM) | (nM) | % |
| AUS-T1000 | 689 | TACAAatgggatgctACTGC | 5-10-5 | 3.4 | 10.6 | 82.7% |
| AUS-T1001 | 690 | TACAAatggGatgctACTGC | 5-4-1-5-5 | 4.0 | 426.5 | 47.7% |
| AUS-T1002 | 691 | TACAaatggGatgctACTGC | 4-5-1-5-5 | 4.6 | 11.1 | 96.4% |
| AUS-T1003 | 692 | TACAaatggGatgctA+C+TGC | 4-5-1-5-5 | 4.6 | 14.3 | 73.2% |
| AUS-T1004 | 693 | ACACAgtttgaatgaAATAG | 5-10-5 | 25.4 | 153.3 | 41.4% |
| AUS-T1005 | 694 | ACAGTttgaatgaaaTAGCA | 5-10-5 | 3.9 | 20.2 | 91.6% |
| AUS-T1006 | 695 | AGTTTgaatgaaataGCAGG | 5-10-5 | 16.7 | 36.2 | 69.3% |
| AUS-T1007 | 696 | TTTGAatgaaatagcAGGTG | 5-10-5 | 0.4 | 17.7 | 61.3% |

*Motifs show the regions of each sequence from 5′ to 3′ (e.g., W1-G1-W2 or W1-G1-S1-G2-W2), where the separator sequence is shown in a bolded font.

TABLE 9B
Nucleotide sequences targeted to TTR (mus_NM_013697.5)

| | SEQ ID | 5′ Target | 3′ Target | |
|---|---|---|---|---|
| AUS No | NO: | site | site | Sequence (5′-3′) |
| AUS-T1000 | 689 | 688 | 669 | TACAAATGGGATGCTACTGC |
| AUS-T1001 | 690 | 688 | 669 | TACAAATGGGATGCTACTGC |
| AUS-T1002 | 691 | 688 | 669 | TACAAATGGGATGCTACTGC |
| AUS-T1003 | 692 | 688 | 669 | TACAAATGGGATGCTACTGC |
| AUS-T1004 | 693 | 786 | 767 | ACACAGTTTGAATGAAATAG |
| AUS-T1005 | 694 | 784 | 765 | ACAGTTTGAATGAAATAGCA |
| AUS-T1006 | 695 | 782 | 763 | AGTTTGAATGAAATAGCAGG |
| AUS-T1007 | 696 | 780 | 761 | TTTGAATGAAATAGCAGGTG |

### Example 10: Inhibition of Murine AAT Expression in AML12 Cells Using Modified Multi-Segmented Antisense Oligonucleotides

Modified multi-segmented antisense oligonucleotides targeted to an AAT nucleic acid were tested for their effects on AAT mRNA in vitro. Cultured murine hepatocyte cells AML12 at a density of 18,000 cells per well in a 96-well plate were treated with a series dilution from 60 nM of modified multi-segmented antisense oligonucleotide. The reference antisense oligonucleotide (ACCCAATTCAGAAGGAAGGA; SEQ ID NO: 697) was highly conserved in mouse. The reference control was administered subcutaneously to C57BL/6 mice at a dose of 100 mg/kg.

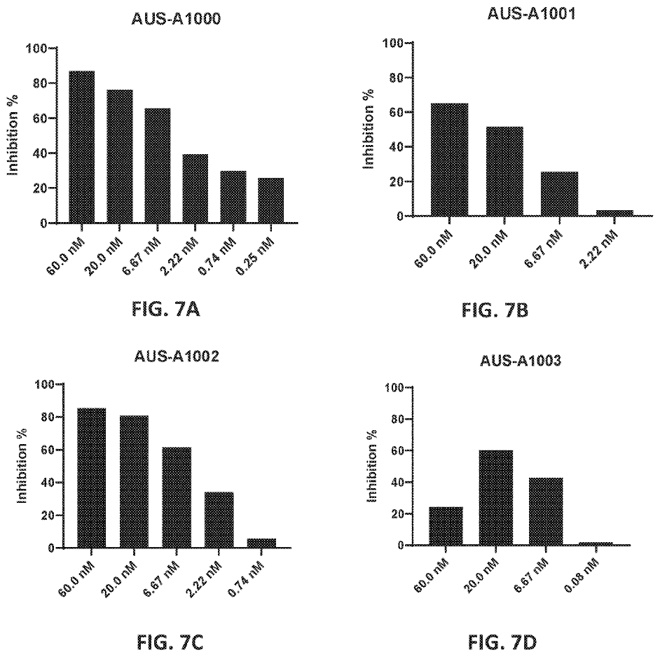

After a treatment period of approximately 48 hours, RNA was isolated from the cells and AAT mRNA levels were measured by quantitative real-time PCR, as described herein. AAT mRNA levels were measured using a mouse AAT primer probe and normalized to housekeeping gene GAPDH mRNA levels. Results are presented as percent inhibition of AAT, relative to untreated control cells (Table 10A and FIGS. 7A-7D). Table 2.2 shows a legend for the modifications at each of the residues shown in Table 10A.

TABLE 10A
Antisense inhibition of AAT in murine cells

| | SEQ | | | | | Maximum |
|---|---|---|---|---|---|---|
| | ID | | | EC₂₅ | EC₅₀ | Inhibition |
| AUS No | NO: | Sequence | Motif* | (nM) | (nM) | % |
| AUS-A1000 | 697 | ACCCAattcagaaggAAGGA | 5-10-5 | 0.6 | 5.6 | 87.2 |
| AUS-A1001 | 698 | ACCCAattcAgaaggAAGGA | 5-4-1-5-5 | 5.1 | 8.9 | 65.0 |
| AUS-A1002 | 699 | ACCCaattcAgaaggAAGGA | 4-5-1-5-5 | 1.6 | 3.2 | 85.3 |
| AUS-A1003 | 700 | ACCCaattcAgaaggA+A+GGA | 4-5-1-5-5 | 4.5 | 3.5 | 60.3 |

*Motifs show the regions of each sequence from 5′ to 3′ (e.g., W1-G1-W2 or W1-G1-S1-G2-W2), where the separator sequence is shown in a bolded font.

### Example 11: Stability of Duplexes Formed with Modified Multi-Segmented Antisense Oligonucleotides

A substitution of a nucleoside with a sugar-modified nucleoside (e.g. 2′-O-MOE) in conventional gapmers results in enhanced binding affinity with a target sequence. This type of enhancement has been explored in conventional gapmer designs to form more stable duplexes with a target sequence, thereby improving the gapmer activity. The stability of the duplexes can be determined by quantifying the melting temperature (Tm). A higher melting temperature is conventionally indicative of enhanced binding with a target sequence and would be expected to result in an increased recruitment of nucleases and functional activity (e.g. silencing of target expression). Experiments were performed to directly compare the melting temperature of modified multi-segmented antisense oligonucleotides (with a separator) to the identical template sequence as a gapmer (without a separator). The stability of the duplexes can be determined by quantifying the melting temperature (Tm).

### Methods

Here, modified multi-segmented antisense oligonucleotides were designed by inserting a separator segment with 2′-modified ribonucleotide into the consecutive stretch of 2′-deoxy ribonucleotide in the gap. Melting temperatures of the modified multi-segmented antisense oligonucleotides were measured. Comparable oligonucleotides (i.e. gapmers designed against the identical target sequence without separator sequences) were used as controls.

### Results

Melting temperatures of oligonucleotides with or without separators are provided in Table 11A, below, along with the sequences and motifs of the tested oligonucleotides.

As described above, the introduction of 2′-modified ribonucleotide was expected to enhance binding affinity with targeted sequence, in turn resulting in an increase of Tm. Unexpectedly, the measured Tm for modified multi-segmented antisense oligonucleotides (with a separator) was lower than the Tm values of the corresponding gapmers (without a separator). The Tm decrease was substantial, with up to a 4° C. decrease.

Melting temperatures were also measured for various modified multi-segmented antisense oligonucleotides targeted to HBV. The results are provided in Table 111B, below. Despite having a reduced melting temperature, the modified multi-segmented antisense oligonucleotides resulted in functional inhibition that was higher than conventional gapmers. For example, modified multi-segmented antisense oligonucleotides having the same structures as AUS1049 (Tm 69.5° C.) and AUS1056 (Tm 70° C.) resulted in 2.18 and 1.87 log 10 maximum reduction in HBsAg compared to 1.15 log 10 by Gapmer AUS1233 (Tm 71.5° C.) when each compound was dosed once on Day 0 at 40 mg/kg by SC in Genotype C 1.0 HBV transgenic mice.

TABLE 11B
Melting temperatures of modified multi-segmented antisense oligonucleotides
targeting HBV

| | SEQ | | | |
|---|---|---|---|---|
| | ID | | | |
| AUS No. | NO: | Sequence | Motif* | Tm (° C.) |
| AUS1233 | 10 | GCAGAggtgaagcgaAGTGC | 5-10-5 | 71.5 |
| (reference) | | | | |
| AUS1049 | 42 | GCAGAggtgAagcgaAGTGC | 5-4-1-5-5 | 69.5 |
| AUS1050 | 43 | GCAGAggtgA′agcgaAGTGC | 5-4-1-5-5 | 71 |
| AUS1051 | 44 | GCAGAggtg+AagcgaAGTGC | 5-4-1-5-5 | 73 |
| AUS1130 | 120 | GCAGAggtgAagcgaAGTGC | 5-4-1-5-5 | 70 |
| AUS1131 | 121 | GCAGAggtgaAgcgaAGTGC | 5-5-1-4-5 | 70 |
| AUS1052 | 45 | GCAGAggtgaagcgAAGTGC | 5-9-6 | 71 |
| AUS1053 | 46 | GCAGAggtgaagcGaAGTGC | 5-8-1-1-5 | 70 |
| AUS1054 | 47 | GCAGAggtgaagCgaAGTGC | 5-7-1-2-5 | 69 |
| AUS1055 | 48 | GCAGAggtgaaGcgaAGTGC | 5-6-1-3-5 | 68.5 |
| AUS1056 | 49 | GCAGAggtgaAgcgaAGTGC | 5-5-1-4-5 | 70 |
| AUS1057 | 50 | GCAGAggtgAagcgaAGTGC | 5-4-1-5-5 | 69.5 |
| AUS1058 | 51 | GCAGAggtGaagcgaAGTGC | 5-3-1-6-5 | 70 |
| AUS1059 | 52 | GCAGAggTgaagcgaAGTGC | 5-2-1-7-5 | 70.5 |
| AUS1060 | 53 | GCAGAgGtgaagcgaAGTGC | 5-1-1-8-5 | 71.5 |
| AUS1061 | 54 | GCAGAGgtgaagcgaAGTGC | 6-9-5 | 73 |
| AUS1062 | 55 | GCAGAgGtgaagcGaAGTGC | 5-1-1-6-1-1-5 | 70 |
| AUS1063 | 56 | GCAGAggTgaagCgaAGTGC | 5-2-1-4-1-2-5 | 67.5 |
| AUS1064 | 57 | GCAGAggtGaaGcgaAGTGC | 5-3-1-2-1-3-5 | 68 |
| AUS1065 | 58 | GCAGAggtgAAgcgaAGTGC | 5-4-2-4-5 | 69.5 |
| AUS1066 | 59 | GCAGAggtgAaGcgaAGTGC | 5-4-1-1-1-3-5 | 68.5 |
| AUS1067 | 60 | GCAGAggtgAagCgaAGTGC | 5-4-1-2-1-2-5 | 67.5 |
| AUS1068 | 61 | GCAGAggtgAagcGaAGTGC | 5-4-1-3-1-1-5 | 68 |
| AUS1069 | 62 | GCAGAggtgAagcgAAGTGC | 5-4-1-4-6 | 69.5 |
| AUS1070 | 63 | GCAGAggtGaAgcgaAGTGC | 5-3-1-1-1-4-5 | 68.5 |
| AUS1071 | 64 | GCAGAggTgaAgcgaAGTGC | 5-2-1-2-1-4-5 | 68.5 |
| AUS1072 | 65 | GCAGAgGtgaAgcgaAGTGC | 5-1-1-3-1-4-5 | 70 |
| AUS1480 | 443 | GCAGAggtgaagcgaA+G+TGC | 5-10-5 | 76 |
| AUS1481 | 444 | GcAGAggtgaagcgaA+G+TGC | 5-10-5 | 75 |
| AUS1490 | 453 | GCAGAggtgAagcgAA+G+TGC | 5-4-1-4-6 | 73 |
| AUS1316 | 289 | GcA+GAggtgAagcgAAGTGC | 5-4-1-4-6 | 72.5 |
| AUS1317 | 290 | GcAG+AggtgAagcgAAGTGC | 5-4-1-4-6 | 71.5 |
| AUS1318 | 291 | +GcAgAggtgAagcgAAGTGC | 5-4-1-4-6 | 68 |
| AUS1319 | 292 | Gc+AgAggtgAagcgAAGTGC | 5-4-1-4-6 | 70 |
| AUS1320 | 293 | GcAg+AggtgAagcgAAGTGC | 5-4-1-4-6 | 70.5 |
| AUS1306 | 279 | GCAG+AGgtgaAgcgaAGTGC | 6-4-1-4-5 | 75 |
| AUS1307 | 280 | +GcAGAGgtgaAgcgaAGTGC | 6-4-1-4-5 | 71.5 |
| AUS1308 | 281 | Gc+AGAGgtgaAgcgaAGTGC | 6-4-1-4-5 | 73.5 |
| AUS1309 | 282 | GcA+GAGgtgaAgcgaAGTGC | 6-4-1-4-5 | 74 |
| AUS1310 | 283 | GcAG+AGgtgaAgcgaAGTGC | 6-4-1-4-5 | 73.5 |
| AUS1311 | 284 | +GcAgAGgtgaAgcgaAGTGC | 6-4-1-4-5 | 70 |
| AUS1312 | 285 | Gc+AgAGgtgaAgcgaAGTGC | 6-4-1-4-5 | 72 |
| AUS1313 | 286 | GcAg+AGgtgaAgcgaAGTGC | 6-4-1-4-5 | 72.5 |
| AUS1302 | 275 | +GCAGAGgtgaAgcgaAGTGC | 6-4-1-4-5 | 73 |
| AUS1303 | 276 | G+CAGAGgtgaAgcgaAGTGC | 6-4-1-4-5 | 74.5 |
| AUS1304 | 277 | GC+AGAGgtgaAgcgaAGTGC | 6-4-1-4-5 | 74.5 |
| AUS1305 | 278 | GCA+GAGgtgaAgcgaAGTGC | 6-4-1-4-5 | 75 |
| AUS1151 | 139 | GCAGAGgtgaAgcgaACTGC | 6-4-1-4-5 | 64 |
| AUS1152 | 140 | GCAGAGgtgaAgcgaAGAGC | 6-4-1-4-5 | 66.5 |
| AUS1153 | 141 | GCAGAGgtgaAgcgaAGTCC | 6-4-1-4-5 | 68.5 |
| AUS1154 | 142 | GCAGAGgtgaAgcgaAGTGG | 6-4-1-4-5 | 69.5 |
| AUS1206 | 189 | CGTGCAGAggtgaagcgaAGTG | 8-10-8 | 81.4 |
| | | CACA | | |
| AUS1209 | 192 | CAGAggtgaagcgaAGTG | 4-10-4 | 65 |
| AUS1244 | 221 | +GC+AGAggtgAagcgaAGTGC | 5-4-1-5-5 | 77 |

*Motifs show the regions of each sequence from 5′ to 3′ (e.g., W1-G1-W2 or W1-G1-S1-G2-W2), where the separator sequence is shown in a bolded font.

The Tm decrease was a general phenomenon observed in multiple sequences that were been evaluated, with vast majority sequences showing more than 2° C. decrease in Tm values. Without wishing to be bound by theory, the significant Tm decrease suggests that the inclusion of a separator and the overall motif of the molecule has a significant impact on the overall conformation of the modified multi-segmented oligonucleotides that lead to an improvement in interaction with the target RNA and or the nucleases (e.g. RNase H) despite the lower Tm value. These data indicate that modified multi-segmented antisense oligonucleotides with separators and motifs as shown provide a new opportunity to increase drug selectivity.

### Example 12: Assessment of Cytotoxicity of Modified Multi-Segmented Antisense Oligonucleotides

Experiments were performed to assess the cytotoxicity of modified multi-segmented antisense oligonucleotides targeting various motifs. Specifically, modified multi-segmented antisense oligonucleotides with separator sequences were compared to corresponding gapmer sequences that lacked separator sequences.

HepG2.2.15 cells were treated with modified multi-segmented antisense oligonucleotides, as described in Example 1. The identities and motifs of the tested modified multi-segmented antisense oligonucleotides are provided in Tables 12A-12E, below, and the sequences of the modified multi-segmented antisense oligonucleotides are provided in Table 11B. The Tm data from Table 11B is also reproduced in Tables 12A-12E, for reference.

Cell viability was determined by MTT cell proliferation assays, as described in Example 1.

TABLE 12A
Viability of cells treated with modified multi-segmented antisense
oligonucleotides targeting HBV

| | SEQ | | MTT CC25 | MTT | Fold CC25 | |
|---|---|---|---|---|---|---|
| | ID | | (nM) | CC25 | over | Tm |
| AUS No. | NO: | Motif* | (Mean) | (CV %) | control | (° C.) |
| AUS1233 | 10 | 5-10-5 | 14.9 | 109.1 | 1.00 | 71.5 |
| AUS1049 | 42 | 5-4-1-5-5 | 31.5 | 119.7 | 2.11 | 69.5 |
| AUS1050 | 43 | 5-4-1-5-5 | 24.6 | 123.8 | 1.65 | 71 |
| AUS1051 | 44 | 5-4-1-5-5 | 31.9 | 116.5 | 2.14 | 73 |

As shown in Table 12A, modified multi-segmented antisense oligonucleotides with a 5-4-1-5-5 motif produced higher CC25 levels in the MTT assay compared to a control gapmer sequence that did not have a separator, but had the same wing structure (AUS1233), under the same experimental conditions. This indicated that the modified multi-segmented antisense oligonucleotides with the 5-4-1-5-5 motif increased cellular viability and were therefore less cytotoxic. Notably, the three 5-4-1-5-5 modified multi-segmented antisense oligonucleotides had different chemical modifications in the separator sequence (specifically, a 2′ MOE separator in AUS 1049, a 2′-F separator in AUS 1050, and a LNA separator in AUS1051).

TABLE 12B
Viability of cells treated with modified multi-segmented
antisense oligonucleotides targeting HBV

| | | | MTT CC25 | |
|---|---|---|---|---|
| | | | (nM) | Tm |
| AUS No. | SEQ ID NO: | Motif* | (Mean) | (° C.) |
| AUS1233 | 10 | 5-10-5 | 22.5 | 71.5 |
| AUS1130 | 120 | 5-4-1-5-5 | 31.9 | 70 |
| AUS1131 | 121 | 5-5-1-4-5 | 40.4 | 70 |

As shown in Table 12B, modified multi-segmented antisense oligonucleotides with a 5-4-1-5-5 or 5-5-1-4-5 motif produced higher CC25 levels in the MTT assay compared to a control gapmer sequence that did not have a separator, but had the same wing structure (AUS1233), under the same experimental conditions. Further, the modified multi-segmented antisense oligonucleotides with separators had lower Tm values compared to the control gapmer sequence. Here, both AUS 1130 and AUS 1131 modified multi-segmented antisense oligonucleotides had 2′-O-Methyl separators.

TABLE 12C
Viability of cells treated with modified multi-segmented antisense
oligonucleotides targeting HBV

| | SEQ | | MTT CC25 | MTT | |
|---|---|---|---|---|---|
| | ID | | (nM) | CC25 | Tm |
| AUS No. | NO: | Motif* | (Mean) | (CV %) | (° C.) |
| AUS1233 | 10 | 5-10-5 | 47.8 | 16.7 | 71.5 |
| AUS1052 | 45 | 5-9-6 | 46.3 | 50.0 | 71 |
| AUS1061 | 54 | 6-9-5 | 32.2 | 59.3 | 73 |
| AUS1056 | 49 | 5-5-1-4-5 | 59.2 | 24.2 | 70 |
| AUS1062 | 55 | 5-1-1-6-1-1-5 | 81.9 | 48.6 | 70 |
| AUS1063 | 56 | 5-2-1-4-1-2-5 | 73.5 | 30.0 | 67.5 |
| AUS1064 | 57 | 5-3-1-2-1-3-5 | 95.8 | 32.1 | 68 |
| AUS1065 | 58 | 5-4-2-4-5 | 64.9 | 18.6 | 69.5 |
| AUS1066 | 59 | 5-4-1-1-1-3-5 | 89.3 | 49.2 | 68.5 |
| AUS1067 | 60 | 5-4-1-2-1-2-5 | 62.7 | 37.1 | 67.5 |
| AUS1068 | 61 | 5-4-1-3-1-1-5 | 58.5 | — | 68 |
| AUS1070 | 63 | 5-3-1-1-1-4-5 | 55.9 | 1.5 | 68.5 |
| AUS1071 | 64 | 5-2-1-2-1-4-5 | 91.5 | 55.7 | 68.5 |
| AUS1072 | 65 | 5-1-1-3-1-4-5 | 92.1 | 58.1 | 70 |

As shown in Table 12C, AUS1061, a gapmer with additional modified nucleotides and a 6-9-5 motif had higher Tm values, indicating a higher affinity to their targets, and lower CC25 in MTT assay compared to the 5-10-5 gapmer AUS1233. This indicated that AUS1061 is more cytotoxic than AUS1233. In contrast, the modified multi-segmented antisense oligonucleotides with separator sequences (AUS1061 to AUS1072) had lower Tm values, indicating lower target-binding affinity, and higher CC25 values in the MTT assay compared to the corresponding control gapmer sequence that did not have a separator (AUS1233). This indicates that the modified multi-segmented antisense oligonucleotides with separators having a variety of motifs were less cytotoxic than the gapmer control.

TABLE 12D
Viability of cells treated with modified multi-segmented antisense
oligonucleotides targeting HBV

| | | | MTT CC25 | MTT | |
|---|---|---|---|---|---|
| | SEQ | | (nM) | CC25 | Tm |
| AUS No. | ID NO: | Motif* | (Mean) | (CV %) | (° C.) |
| AUS1480 | 443 | 5-10-5 | 41.1 | 27.0 | 76 |
| AUS1490 | 453 | 5-4-1-4-6 | 57.5 | 12.2 | 73 |

As shown in Table 12D, the modified multi-segmented antisense oligonucleotide AUS1490 had a lower Tm (indicating lower target-binding affinity) and a higher CC25 level in the MTT assay compared to the corresponding control gapmer sequence that did not have a separator but had the same W2 modification (AUS1480). This indicated that the modified multi-segmented antisense oligonucleotide with the separator was less cytotoxic than the gapmer control.

TABLE 12E
Viability of cells treated with modified multi-segmented antisense
oligonucleotides targeting HBV

| | SEQ | | MTT CC25 | MTT CC25 | Tm |
|---|---|---|---|---|---|
| AUS No. | ID NO: | Motif* | (nM) (Mean) | (CV %) | (° C.) |
| AUS1481 | 444 | 5-10-5 | 34.0 | 22.9 | 75 |
| AUS1316 | 289 | 5-4-1-4-6 | 58.5 | 46.2 | 72.5 |
| AUS1317 | 290 | 5-4-1-4-6 | 50.4 | 40.6 | 71.5 |
| AUS1318 | 291 | 5-4-1-4-6 | 49.0 | 43.6 | 68 |
| AUS1319 | 292 | 5-4-1-4-6 | 48.0 | 38.2 | 70 |
| AUS1320 | 293 | 5-4-1-4-6 | 58.9 | 48.7 | 70.5 |
| AUS1312 | 285 | 6-4-1-4-5 | 51.8 | 39.2 | 72 |
| AUS1313 | 286 | 6-4-1-4-5 | 59.8 | 62.0 | 72.5 |

Finally, as shown in Table 12E, modified multi-segmented antisense oligonucleotides with 5-4-1-4-6 (AUS1316-AUS1320) or 6-4-1-4-5 (AUS1312 and AUS1313) motifs had lower Tm values (indicating lower target-binding affinity) and higher CC25 levels in the MTT assay compared to the corresponding control gapmer sequence that did not have a separator but had the same W2 modification (AUS1481). This indicates that the modified multi-segmented antisense oligonucleotides with separators were less cytotoxic than the gapmer control.

### Example 13: RNase H Cleavage Assay

Antisense oligonucleotide-directed RNase H1 cleavage of the target RNA was evaluated in vitro. A gapmers or a modified multi-segmented antisense oligonucleotide (1.25 pmol each) was mixed with 12.5 pmol of the target RNA in saline to a total volume of 15 μL. RNase H enzyme was added and the samples were incubated at 37° C. for 1 h. The mixture was rapidly heated to 95° C. for 5 min then cooled to 4° C. RNase H cleavage products were analyzed in 20% Precast-UREA-TBE PAGE gel. The gels were stained using SYBR gold and photographed using Molecular Imager FX (Bio-Rad, USA).

The gel bands at approximately 50K in Lanes 1-9 are the target RNA. The bands above the target RNA band are oligonucleotide+target RNA complexes. The bands below the target RNA band are the degradation products after incubation with RNase H.

The oligonucleotides used in Lane 1 to Lane 7 have the same target RNA. AUS1233, AUS1013, AUS1014, AUS1015, and AUS1212 are gapmers with motifs of 5-10-5, 9-2-9, 8-4-8, 6-8-6, 5-10-10, respectively. AUS1493 is a multi-segmented oligonucleotide with a motif of 4-5-1-5-5. As shown in the gel image, while the gapmers to the same target RNA have similar cleavage patterns (Lanes 1, 6, and 7), the multi-segmented oligonucleotide (Lane 2 and Lane 3) has a different cleavage pattern than the gapmers to the same target RNA. Consistent with the conventional knowledge in the field, gapmers with gap sizes of 2 (AUS1013) and 4 (AUS1014) did not show observable cleavage products (Lanes 4 and 5).

### Example 14: Antisense Inhibition of Murine PCSK9 in Wild Type C57BL/6 Mice

Male C57BL/6 mice (a lean mouse model) at weight of 20-22 g were used in the instant experiment. After a week acclimation period, mice were randomly divided into five groups (four mice each). Modified multi-segmented antisense oligonucleotides were prepared in saline for subcutaneous injection. Wild type mice received modified multi-segmented antisense oligonucleotides at 50 mg/kg once a week for 2 weeks. ISIS 394816 (AUS-P1000) was tested as a reference control. After treatment with modified multi-segmented antisense oligonucleotides, changes in serum PCSK9 protein levels were measured by ELISA. Each treatment group consisted for 4 animals.

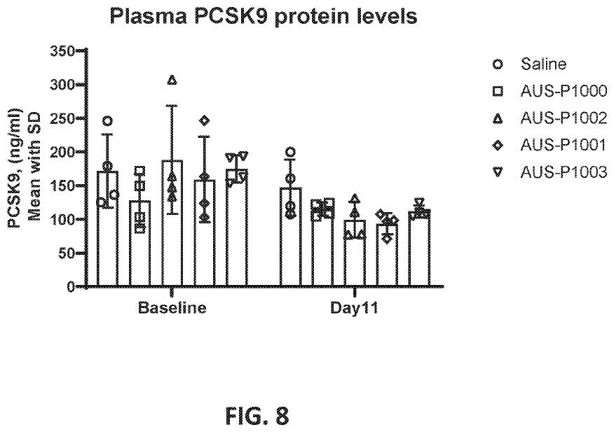

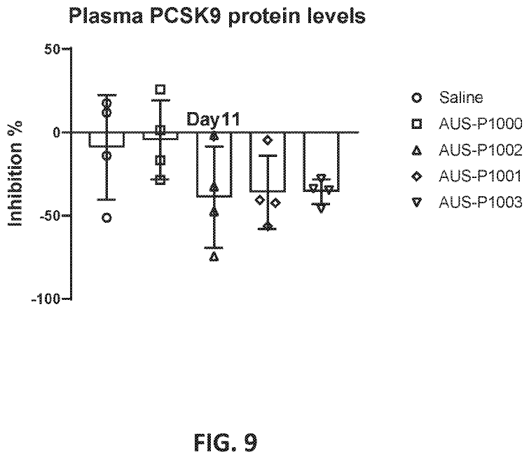

Results: Inhibition of serum PCSK9 levels—The results for the novel modified multi-segmented antisense oligonucleotides tested are shown in Tables 14A and 14B, FIG. 8 and FIG. 9. Table 2.2 shows a legend for the modifications at each of the residues shown in Table 14A.

After two doses of the reference oligonucleotide ISIS 394816 at 50 mg/kg in the lean mouse model, it does not reduce the plasma PCSK9 protein level. The results were consistent with what was described U.S. Pat. No. 9,650,636 B2 and WO2009148605A) and that multiple doses are necessary to achieve the optimal efficacy in hyperlipidemic mouse model. However, the instant results showed several of the novel modified multi-segmented antisense oligonucleotides were more potent than the reference control ISIS 394816 (AUS-P1000).

TABLE 14A
Antisense inhibition of PCSK9 in mouse

| AUS No | SEQ ID NO: | Sequence | Motif* |
|---|---|---|---|
| AUS-P1000 | 667 | GGGCTcatagcacatTATCC | 5-10-5 |
| AUS-P1001 | 668 | GGGCtcataGcacatTATCC | 4-5-1-5-5 |
| AUS-P1002 | 669 | GGGCTcataGcacatTATCC | 5-4-1-5-5 |
| AUS-P1003 | 670 | GGGCtcataGcacatT+A+TCC | 4-5-1-5-5 |

*Motifs show the regions of each sequence from 5′ to 3′ (e.g., W1-G1-W2 or W1-G1-S1-G2-W2), where the separator sequence is shown in a bolded font.

TABLE 14B
Antisense inhibition of serum PCSK9 protein level in mouse

| | SEQ | PCSK9 | | |
|---|---|---|---|---|
| AUS No | ID NO: | Day 1 (ng/ml) | Day 11 (ng/ml) | Inhibition % ± SD |
| Saline | | 165.6 ± 47.3 | 142.6 ± 36.2 | 9 ± 31.3 |
| AUS-P1000 | 667 | 123.1 ± 34.3 | 114.8 ± 8.6 | 4.6 ± 23.6 |
| AUS-P1001 | 668 | 177.7 ± 69.6 | 97.0 ± 22.7 | 39.0 ± 30.3 |
| AUS-P1002 | 669 | 150.6 ± 54.9 | 92.5 ± 13.6 | 36.0 ± 22.0 |
| AUS-P1003 | 670 | 174.1 ± 17.5 | 111.3 ± 7.7 | 35.7 ± 7.5 |

### Example 15: Antisense Inhibition of Murine TTR in Wild Type C57BL/6 Mice

Male C57BL/6 mice at weight of 20-22 g were used. After a week acclimation period, mice were randomly divided into five groups (four mice each). Modified multi-segmented antisense oligonucleotides were prepared in saline for subcutaneous injection. Wild type mice received modified multi-segmented antisense oligonucleotides at 50 mg/kg once a week for 2 weeks. ISIS 401724 (AUS-T1000) previously described in U.S. Pat. No. 8,541,388 B2, was tested as a reference control. After treatment with modified multi-segmented antisense oligonucleotides, changes in serum TTR protein levels were measured by ELISA. Each treatment group consisted for 4 animals.

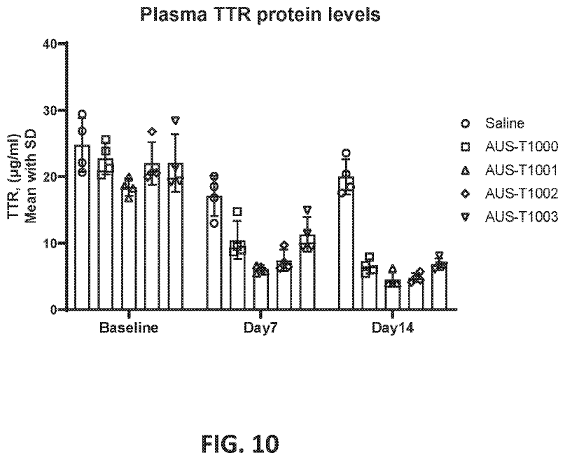

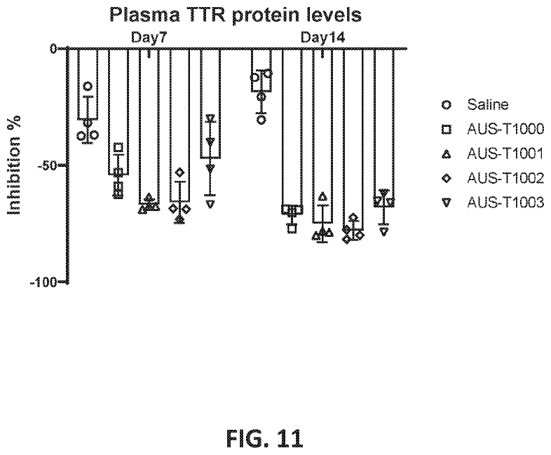

Results: Inhibition of serum TTR levels—The novel modified multi-segmented antisense oligonucleotides that were tested are shown in Table 15A. The reference oligonucleotide AUS-T000 showed a 23.6% inhibition in plasma TTR protein levels on day 7 after single dose of 50 mg/kg. The second dose of AUS-T1000 could inhibit 56.54 plasma TTR protein levels on day 14. The results showed several of the newly designed modified multi-segmented antisense oligonucleotides were more potent than the reference control ISIS 401724 (AUS-T1000) (Tables 5A and 15B, FIGS. 10-11). Table 2.2 shows a legend for the modifications at each of the residues shown in Table 15A.

TABLE 15A
Antisense inhibition of TTR in mouse

| AUS No | SEQ ID NO: | Sequence | Motif* |
|---|---|---|---|
| AUS-T1000 | 689 | TACAAatgggatgctACTGC | 5-10-5 |
| AUS-T1001 | 690 | TACAAatggGatgctACTGC | 5-4-1-5-5 |
| AUS-T1002 | 691 | TACAaatggGatgctACTGC | 4-5-1-5-5 |
| AUS-T1003 | 692 | TACAaatggGatgctA+C+TGC | 4-5-1-5-5 |

*W1-G1-W2 or W1-G1-S1-G2-W2; separator position is bolded.

TABLE 15B
Antisense inhibition of TTR in mouse

| | | TTR (μg/mL) | | |
|---|---|---|---|---|
| AUS No | SEQ ID NO: | Day 1 | Day 7 | Day 14 |
| Saline | — | 24.5 ± 3.5 | 16.9 ± 2.6 | 19.9 ± 2.3 |
| AUS-T1000 | 689 | 22.6 ± 2.1 | 10.2 ± 2.5 | 6.5 ± 0.9 |
| AUS-T1001 | 690 | 18.4 ± 1.1 | 6.1 ± 0.5 | 4.5 ± 0.9 |
| AUS-T1002 | 691 | 21.8 ± 2.8 | 7.3 ± 1.4 | 4.8 ± 0.6 |
| AUS-T1003 | 692 | 21.8 ± 3.8 | 11.1 ± 2.3 | 6.8 ± 0.8 |

TABLE 15C
The inhibition of plasma TTR protein levels in mouse

| | | Inhibition % ± SD | |
|---|---|---|---|
| AUS No | SEQ ID NO: | Day 7 | Day 14 |
| Saline | — | 30.6 ± 10.0 | 18.5 ± 9.1 |
| AUS-T1000 | 689 | 54.2 ± 8.8 | 75.0 ± 7.9 |
| AUS-T1001 | 690 | 66.9 ± 2.2 | 71.3 ± 4.0 |
| AUS-T1002 | 691 | 65.9 ± 8.8 | 77.9 ± 4.1 |
| AUS-T1003 | 692 | 47.1 ± 15.7 | 68.0 ± 7.3 |

### Example 16: Antiviral Effect of Modified Multi-Segmented Antisense Oligonucleotides in Genotype C HBV Transgenic Mice

A study was performed to evaluate the effect of modified multi-segmented antisense oligonucleotides on the serum levels of HBsAg in genotype C HBV (HBV-GTC) transgenic mice. The modified multi-segmented antisense oligonucleotides used in this study included AUS1169, AUS1171, AUS1170, AUS1168, AUS1322. Each compound was dosed once at 40 mg/kg by subcutaneous injection (SC) to the mice. Normal saline and AUS1233 were administered as a negative control and reference compound, respectively. Sequences and motifs of the modified multi-segmented antisense oligonucleotides are provided in Table 16A.

HBV transgenic mice, prepared from male C57BL/6 mice of 5-7 weeks old with 1.0-fold length of HBV C1 subtype genome, stably express HBsAg in liver. The methods and purposes of animal experiments were in accordance with the ethical standards and international practices.

AUS1233 was used as a reference compound in this study. HBsAg ELISA kits were purchased from Autobio Diagnostics. ELISA was performed using a chemiluminescence immunoassay analyzer (Instrument model: LUMO) at Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 4.0 mg/ml.

Animal Dosing: Male HBV transgenic mice were randomly divided into groups of 4 mice. Each group was dosed with one compound by subcutaneous injection at 40 mg/kg and a dose volume of 10 ml/kg of body weight or an equal volume of normal saline as control.

Approximately 80 μl of blood sample was taken from a thigh vein on 0 (before dosing), 3, 7, 10, 14, 21 and 28 days after dosing. Blood samples were placed in an incubator at 37° C. for approximately 30 minutes, and then centrifuged in a precooled (0-4) centrifuge to obtain the serum samples. The obtained serum samples were stored in a −20° C. refrigerator for further analysis.

Serum HBsAg levels in the serum samples were measured using HBsAg ELISA kits (Autobio Diagnostics). Briefly, 50 μl of 10-fold diluted mouse serum or standards was added to a 96-well plate, and HBsAg level was measured according to manufacturer's instructions.

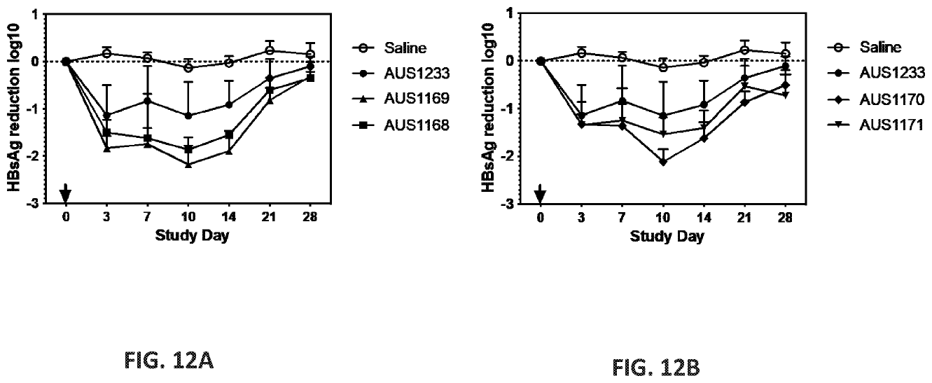

The HBsAg levels were measured in the serum samples by ELISA, and the results are provided in FIGS. 12A-12B and Table 16B, below.

TABLE 16A
Oligonucleotides tested for antisense inhibition of HBV in mouse

| AUS No | SEQ ID NO: | Sequence | Motif* |
|---|---|---|---|
| AUS1233 | 10 | GCAGAggtgaagcgaAGTGC | 5-10-5 |
| AUS1169 | 155 | GCAGAggtgAagcgaAGTGC | 5-4-1-5-5 |
| AUS1168 | 154 | GCAGAggtgaAgcgaAGTGC | 5-5-1-4-5 |
| AUS1170 | 156 | GCAGAggtgAagcgAAGTGC | 5-4-1-4-6 |
| AUS1171 | 157 | GCAGAGgtgaAgcgaAGTGC | 6-4-1-4-5 |
| AUS1322 | 294 | +GC+AGA ggtgA agcga AGTGC | 5-4-1-5-5 |

*Motifs show the regions of each sequence from 5′ to 3′ (e.g., W1-G1-W2 or W1-G1-S1-G2-W2), where the separator sequence is shown in a bolded font.

TABLE 16B
Reduction of HBsAg following treatment with modified multi-segmented
antisense oligonucleotides

| AUS No. | | | AUS1233 | AUS1169 | AUS1168 | AUS1170 | AUS1171 | AUS1322 |
|---|---|---|---|---|---|---|---|---|
| SEQ ID NO: | | | 10 | 155 | 154 | 156 | 157 | 294 |
| Days | 3 | mean | −1.13 | −1.83 | −1.50 | −1.33 | −1.34 | −2.33 |
| post- | | SD | 0.64 | 0.26 | 0.27 | 0.20 | 0.48 | 0.14 |
| dosing | 7 | mean | −0.83 | −1.75 | −1.62 | −1.36 | −1.25 | −2.32 |
| | | SD | 0.73 | 1.06 | 0.21 | 0.79 | 0.35 | 0.16 |
| | 10 | mean | −1.15 | −2.18 | −1.87 | −2.11 | −1.54 | −2.11 |
| | | SD | 0.71 | 0.40 | 0.27 | 0.26 | 0.36 | 0.11 |
| | 14 | mean | −0.92 | −1.90 | −1.55 | −1.62 | −1.40 | −1.98 |
| | | SD | 0.50 | 0.43 | 0.08 | 0.34 | 0.37 | 0.19 |
| | 21 | mean | −0.36 | −0.82 | −0.60 | −0.86 | −0.53 | −1.69 |
| | | SD | 0.41 | 0.26 | 0.26 | 0.23 | 0.43 | 0.29 |
| | 28 | mean | −0.10 | −0.32 | −0.35 | −0.50 | −0.72 | −0.98 |
| | | SD | 0.20 | 0.30 | 0.14 | 0.22 | 0.53 | 0.17 |

| | | | | | | |
|---|---|---|---|---|---|---|
| Max Reduction | −1.15 | −2.18 | −1.87 | −2.11 | −1.54 | −2.33 |

The maximum log 10 serum HBsAg reduction was −1.151±0.71 for AUS1233, −2.18±0.40 for AUS 1169, −1.54±0.36 for AUS 1171, −2.11±10.26 for AUS 1170, −1.87±0.27 for AUS 1168, and −2.32±0.16 for AUS 1322. The body weight change was comparable with the saline group for all treatment groups. The results showed that the antiviral effects of the present modified multi-segmented antisense oligonucleotides were much more effective in vivo than the corresponding gapmer AUS1233.

### Example 17: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HBsAg in HDI-HBV GTD carrier mice. The modified multi-segmented antisense oligonucleotides used in this study included AUS1322 and AUS1324; the sequences and motifs of these modified multi-segmented antisense oligonucleotides are provided in Table 17A. Each compound was dosed once at 60 mg/kg by SC. Normal saline was administered as negative control.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HBsAg-positive mice were used Day 11 after the injection. The mice were maintained under specific pathogen-free conditions in the BSL-2+ animal facility of Hagnzhou Eyoung biotechnology Co., Ltd (China). The methods and purposes of animal experiments were in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 6.0 mg/ml.

Male HDI-HBV GTD carrier mice were randomly divided into each group of 3 to 5 mice. Each group was dosed with one compound at 60 mg/kg and a dose volume of 10 ml/kg of body weight or an equal volume of normal saline as control. HBsAg-positive mice were dosed 11 days after HDI modeling at the animal laboratory of Eyoung via SC.

Approximately 80 μl of blood sample was taken from a thigh vein on 0 (before dosing), 3, 7, 10, 14, 21 and 28 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis.

Serum HBsAg levels in the serum samples were measured using HBsAg ELISA kits (Autobio Diagnostics, China). Briefly, 50 μl of 25-fold diluted mouse serum or standards was added to a 96-well plate, HBsAg level was measured according to manufacturer's instructions.

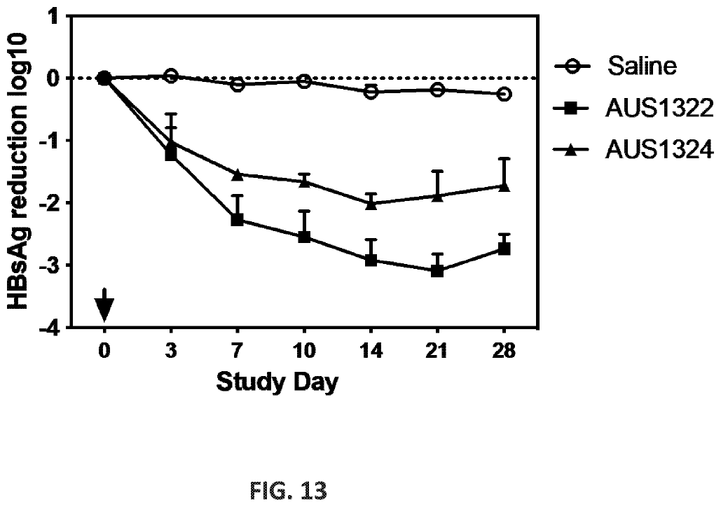

Results are provided in FIG. 13 and Table 17B, below.

TABLE 17A
Oligonucleotides tested for antisense inhibition of HBV in mouse

| AUS No | SEQ ID NO: | Sequence | Motif* |
|---|---|---|---|
| AUS1324 | 296 | +GC+AGAggtgaagcgaAGTGC | 5-4-1-5-5 |
| AUS1322 | 294 | +GC+AGAggtgAagcgaAGTGC | 5-4-1-5-5 |

*Motifs show the regions of each sequence from 5′ to 3′ (e.g., W1-G1-W2 or W1-G1-S1-G2-W2), where the separator sequence is shown in a bolded font.

TABLE 17B
Reduction of HBsAg following treatment with modified
multi-segmented antisense oligonucleotides

| AUS No. | | | AUS1322 | AUS1324 |
|---|---|---|---|---|
| SEQ ID NO: | | | 294 | 296 |
| Days | 3 | mean | −1.22 | −1.03 |
| post- | | SD | 0.42 | 0.45 |
| dosing | 7 | mean | −2.27 | −1.54 |
| | | SD | 0.38 | 0.07 |
| | 10 | mean | −2.54 | −1.66 |
| | | SD | 0.41 | 0.12 |
| | 14 | mean | −2.92 | −2.01 |
| | | SD | 0.33 | 0.15 |
| | 21 | mean | −3.09 | −1.88 |
| | | SD | 0.26 | 0.39 |
| | 28 | mean | −2.74 | −1.73 |
| | | SD | 0.24 | 0.43 |

| | | |
|---|---|---|
| Max Reduction | −3.09 | −2.01 |

The maximum log 10 serum HBsAg reduction was −3.09±0.26 log 10 for the modified multi-segmented antisense oligonucleotides AUS1322, which is much better efficacy than the −2.01±10.15 log 10 HBsAg reduction by the corresponding gapmer AUS1324. The body weight change was comparable with the saline group for all treatment groups.

### Example 18: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HBsAg in HDI-HBV GTD carrier mice. The modified multi-segmented antisense oligonucleotides used in this study included AUS1169 and AUS1170; the sequences and motifs of these modified multi-segmented antisense oligonucleotides are provided in Example 16, above. Each compound was dosed once at 60 mg/kg by subcutaneous injection (SC) to the mice. Normal saline and AUS1233 were administered as negative control and gapmer reference compound, respectively.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HBsAg-positive mice were used Day 40 after the injection. The mice were maintained under specific pathogen-free conditions in the BSL-2+ animal facility of Hagnzhou Eyoung biotechnology Co., Ltd (China). The methods and purposes of animal experiments were in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 6.0 mg/ml.

Male HDI-HBV GTD carrier mice were randomly divided into each group of 3 to 4 mice. Each group was dosed with one compound at 60 mg/kg and a dose volume of 10 ml/kg of body weight or an equal volume of normal saline as control. HBsAg-positive mice were dosed 40 days after HDI modeling at the animal laboratory of Eyoung via SC.

Approximately 80 μl of blood sample was taken from a thigh vein on 0 (before dosing), 3, 7, 10, 14, 21 and 28 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis. Serum HBsAg levels were measured as described in Example 17, above.

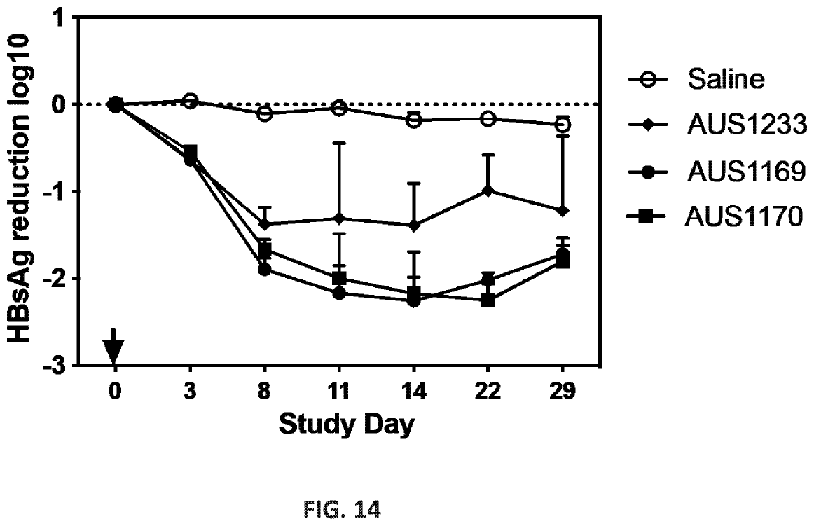

Results are provided in FIG. 14 and Table 18, below.

TABLE 18A
Reduction of HBsAg following treatment with modified
multi-segmented antisense oligonucleotides

| AUS No. | | | AUS1233 | AUS1169 | AUS1170 |
|---|---|---|---|---|---|
| SEQ ID NO: | | | 10 | 155 | 156 |
| Days | 3 | mean | −0.82 | −0.63 | −0.54 |
| post- | | SD | 0.32 | 0.03 | 0.01 |
| dosing | 7 | mean | −1.45 | −1.89 | −1.67 |
| | | SD | 0.19 | 0.13 | 0.12 |
| | 10 | mean | −1.37 | −2.17 | −2.00 |
| | | SD | 0.63 | 0.32 | 0.51 |
| | 14 | mean | −1.48 | −2.26 | −2.17 |
| | | SD | 0.39 | 0.27 | 0.48 |
| | 21 | mean | −0.95 | −2.02 | −2.25 |
| | | SD | 0.30 | 0.08 | 0.19 |
| | 28 | mean | −0.97 | −1.72 | −1.80 |
| | | SD | 0.70 | 0.10 | 0.27 |

| | | | |
|---|---|---|---|
| Max Reduction | −1.48 | −2.26 | −2.25 |

The maximum log 10 serum HBsAg reduction for modified multi-segmented oligonucleotide AUS 1169 and AUS 1170 was −2.26±0.27 and −2.25±0.19, respectively, which are much better efficacy than the −1.48±0.39 log 10 HBsAg reduction by the corresponding control gapmer AUS1233. The body weight change was comparable with the saline group for all treatment groups.

### Example 19: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HbsAg in HDI-HBV GTD carrier mice. The modified multi-segmented antisense oligonucleotides used in this study included AUS 1173, AUS 1174, and AUS 1175; the sequences and motifs of these modified multi-segmented antisense oligonucleotides are provided in Table 19A, below. Each compound was dosed once at 60 mg/kg by subcutaneous injection (SC) to the mice. Normal saline and AUS1233 were administered as negative control and positive control, respectively.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HbsAg-positive mice were used Day 22 after the injection. The mice were maintained under specific pathogen-free conditions in the BSL-2+ animal facility of Hagnzhou Eyoung biotechnology Co., Ltd (China). The methods and purposes of animal experiments are in accordance with the ethical standards and international practices.

HbsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 6.0 mg/ml.

Male HDI-HBV GTD carrier mice were randomly divided into each group of 3 to 5 mice. Each group was dosed with one compound at 60 mg/kg and a dose volume of 10 ml/kg of body weight or an equal volume of normal saline as control. HbsAg-positive mice were dosed 22 days after HDI modeling at the animal laboratory of Eyoung via SC.

Approximately 80 μl of blood sample was taken from a thigh vein on 0 (before dosing), 3, 7, 10, 14 and 21 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis. Serum HbsAg levels were measured as described in Example 17, above.

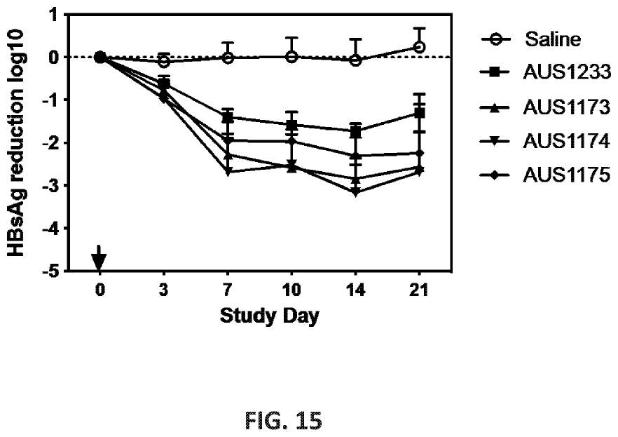

Results are provided in FIG. 15 and Table 19B, below.

TABLE 19A
Oligonucleotides tested for antisense inhibition of HBV in mouse

| AUS No | SEQ ID NO: | Sequence | Motif* |
|---|---|---|---|
| AUS1233 | 10 | GCAGAggtgaagcgaAGTGC | 5-10-5 |
| AUS1173 | 158 | GCAGAggtgAagcgaAGTGC | 5-4-1-5-5 |
| AUS1174 | 159 | GCAGAggtgAagcgaAGTGC | 5-4-1-5-5 |
| AUS1175 | 160 | GCAGagtgAagcgaAGTGC | 4-5-1-5-5 |
| AUS1176 | 161 | GCAgaggtgAagcgaAGTGC | 3-5-1-5-5 |

*Motifs show the regions of each sequence from 5′ to 3′ (e.g., W1-G1-W2 or W1-G1-S1-G2-W2), where the separator sequence is shown in a bolded font.

TABLE 19B
Reduction of HBsAg following treatment with
modified multi-segmented antisense oligonucleotides

| AUS No. | | | AUS1233 | AUS1173 | AUS1174 | AUS1175 | AUS1176 |
|---|---|---|---|---|---|---|---|
| SEQ ID NO: | | | 10 | 158 | 159 | 160 | 161 |
| Days | 3 | mean | −0.55 | −0.74 | −0.96 | −0.99 | −0.49 |
| post-dosing | | SD | 0.11 | 0.13 | 0.35 | 0.60 | 0.07 |
| | 7 | mean | −1.37 | −2.13 | −2.68 | −2.07 | −1.39 |
| | | SD | 0.20 | 0.22 | 0.90 | 0.58 | 0.36 |
| | 10 | mean | −1.46 | −2.27 | −2.52 | −1.96 | −1.29 |
| | | SD | 0.16 | 0.39 | 0.53 | 0.34 | 0.40 |
| | 14 | mean | −1.73 | −2.84 | −3.17 | −2.07 | −1.17 |
| | | SD | 0.17 | 0.48 | 0.66 | 0.35 | 0.44 |
| | 21 | mean | −1.30 | −2.22 | −2.68 | −1.24 | −0.53 |
| | | SD | 0.21 | 0.37 | 0.93 | 0.22 | 0.32 |

| | | | | | |
|---|---|---|---|---|---|
| Max Reduction | −1.73 | −2.84 | −3.17 | −2.07 | −1.39 |

The maximum log 10 serum HBsAg reduction was −1.73±0.17 for the gapmer AUS1233, −2.84±0.48 for AUS 1173, −2.68±0.90 for AUS 1174, −2.07±0.58 for AUS 1175. The body weight change was comparable with the saline group for all treatment groups. The results showed that the antiviral effects of the present modified multi-segmented antisense oligonucleotides AUS1173, AUS1174 and AUS1175 were better than the corresponding gapmer AUS1233.

### Example 20: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HBsAg in HDI-HBV GTD carrier mice. The modified multi-segmented antisense oligonucleotides used in this study included AUS 1178, AUS 1188, AUS 1175, AUS 1190, AUS 1191, and AUS 1192; sequences and motifs of these modified multi-segmented antisense oligonucleotides are provided in Table 20A, below. Each compound was dosed once at 60 mg/kg by subcutaneous injection (SC) to the mice. Normal saline was administered as negative control, AUS1233 and AUS 1177 were administered as gapmer reference compounds.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HBsAg-positive mice were used Day 30 after the injection. The mice were maintained under specific pathogen-free conditions in the BSL-2+ animal facility of Hagnzhou Eyoung biotechnology Co., Ltd (China). The methods and purposes of animal experiments are in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 6.0 mg/ml.

Male HDI-HBV GTD carrier mice were randomly divided into each group of 3 to 5 mice. Each group was dosed with one compound at 60 mg/kg and a dose volume of 10 ml/kg of body weight or an equal volume of normal saline as control. HBsAg-positive mice were dosed 30 days after HDI modeling at the animal laboratory of Eyoung via SC.

Approximately 80 μl of blood sample was taken from a thigh vein on 0 (before dosing), 3, 7, 10, and 14 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis. Serum HBsAg levels were measured as described in Example 17, above.

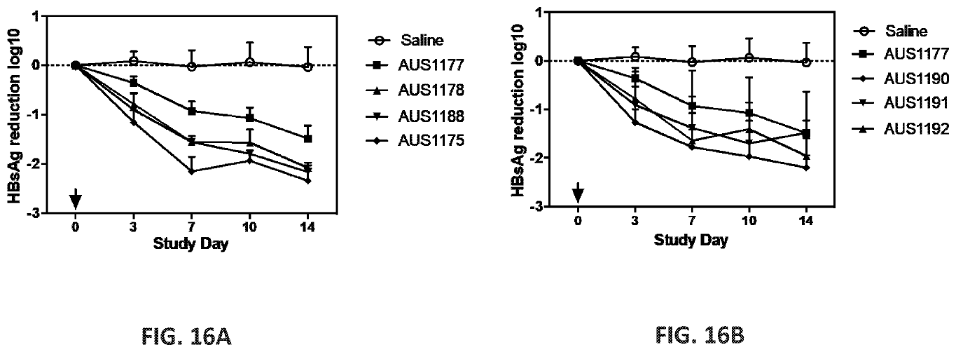

Results are provided in FIGS. 16A-16B and Table 20B, below.

TABLE 20A
Oligonucleotides tested for antisense inhibition of HBV in mouse

| AUS No | SEQ ID NO: | Sequence | Motif* |
|---|---|---|---|
| AUS1177 | 162 | GCAGAggtgaagcgaAGUGC | 5-10-5 |
| AUS1178 | 163 | GCAGAggtgAagcgaAGUGC | 5-4-1-5-5 |
| AUS1188 | 173 | GCAGAggtgAagcgAAGUGC | 5-4-1-4-6 |
| AUS1190 | 175 | GCAGAggtgAagcgaAGUGC | 5-4-1-5-5 |
| AUS1191 | 176 | GCAGAggtgaAgcgaAGUGC | 5-5-1-4-5 |
| AUS1192 | 177 | GCAGAggtgAagcgAAGUGC | 5-4-1-4-6 |
| AUS1175 | 160 | GCAGaggtgAagcgaAGTGC | 4-5-1-5-5 |
| AUS1183 | 168 | GCAGAggtgaAgcgaAGUGC | 5-5-1-4-5 |

*Motifs show the regions of each sequence from 5′ to 3′ (e.g., W1-G1-W2 or W1-G1-S1-G2-W2), where the separator sequence is shown in a bolded font.

TABLE 20B
Reduction of HbsAg following treatment with modified multi-segmented
antisense oligonucleotides

| AUS | | | AUS | AUS | AUS | AUS | AUS | AUS | AUS | AUS |
|---|---|---|---|---|---|---|---|---|---|---|
| No. | | | 1177 | 1178 | 1188 | 1190 | 1191 | 1192 | 1175 | 1183 |
| SEQ ID NO: | | | 162 | 163 | 173 | 175 | 176 | 177 | 160 | 168 |
| Days | 3 | mean | −0.36 | −0.79 | −0.91 | −1.27 | −0.91 | −0.96 | −1.16 | −0.96 |
| post- | | SD | 0.13 | 0.23 | 0.34 | 0.27 | 0.77 | 0.41 | 0.24 | 0.57 |
| dosing | 7 | mean | −0.93 | −1.56 | −1.56 | −1.78 | −1.38 | −2.29 | −2.15 | −1.99 |
| | | SD | 0.19 | 0.13 | 0.06 | 0.36 | 1.19 | 1.37 | 0.29 | 1.36 |
| | 10 | mean | −1.07 | −1.57 | −1.80 | −1.97 | −1.70 | −1.39 | −1.94 | −1.79 |
| | | SD | 0.22 | 0.27 | 0.05 | 0.74 | 1.36 | 0.26 | 0.22 | 1.11 |
| | 14 | mean | −1.48 | −2.08 | −2.17 | −2.20 | −1.48 | −1.96 | −2.35 | −1.93 |
| | | SD | 0.26 | 0.11 | 0.14 | 0.25 | 0.84 | 0.36 | 0.25 | 1.08 |

| | | | | | | | | |
|---|---|---|---|---|---|---|---|---|
| Max Reduction | −1.48 | −2.08 | −2.17 | −2.20 | −1.70 | −2.40 | −2.35 | −1.99 |

The maximum log 10 serum HBsAg reduction was −1.481±0.26 for AUS1177, −2.08±0.11 for AUS11178, −2.17±L0.14 for AUS1188, −2.35±10.25 for AUS1175, −2.20±0.25 for AUS1190, −1.70±1.36 for AUS1191, and −1.96±0.36 for AUS1192. The body weight change was comparable with the saline group for all treatment groups. The results showed that the antiviral effects of the present modified multi-segmented antisense oligonucleotides were much better than the positive control AUS 1233 and AUS 1177.

### Example 21: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HBsAg in HDI-HBV GTD carrier mice. The modified multi-segmented antisense oligonucleotides used in this study included AUS 1464, and AUS 1488; the sequences and motifs for these modified multi-segmented antisense oligonucleotides are provided in Table 21A, below. AUS 1483 was administered as a gapmer reference. Each compound was dosed once at 40 mg/kg by subcutaneous injection (SC) to the mice. Normal saline was administered as negative control.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HBsAg-positive mice were used Day 19 after the injection. The mice were maintained under specific pathogen-free conditions in the BSL-2+ animal facility of Hagnzhou Eyoung biotechnology Co., Ltd (China). The methods and purposes of animal experiments are in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 4.0 mg/ml.

Male HDI-HBV GTD carrier mice were randomly divided into each group of 2 to 3 mice. Each group was dosed with one compound at 40 mg/kg and a dose volume of 10 ml/kg of body weight or an equal volume of normal saline as control. HBsAg-positive mice were dosed 30 days after HDI modeling at the animal laboratory of Eyoung via SC.

Approximately 80 μl of blood sample was taken from a thigh vein on 0 (before dosing), 3, 7, 10, 14 and 21 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis.

Serum HBsAg levels in the serum samples were measured using HBsAg ELISA kits were purchased from Autobio Diagnostics (China). Briefly, 50 μl of 25-fold diluted mouse serum or standards was added to a 96-well plate, HBsAg level was measured according to manufacturer's instructions.

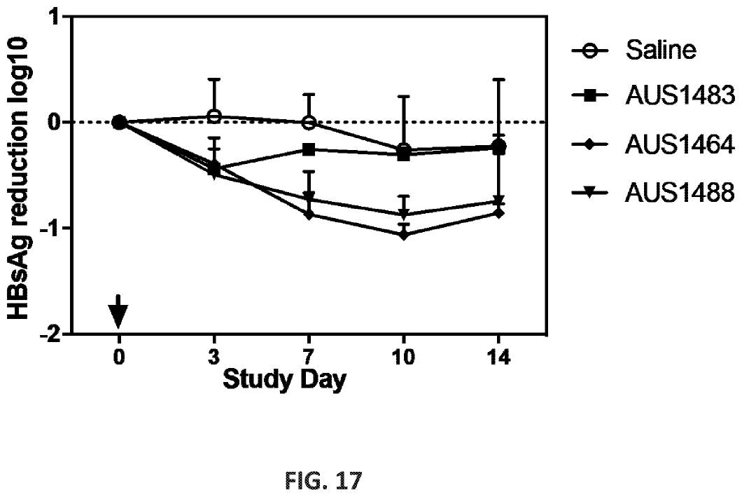

Results are provided in FIG. 17 and Table 21B, below.

TABLE 21A
Oligonucleotides tested for antisense inhibition of HBV in mouse

| AUS No | SEQ ID NO: | Sequence | Motif* |
|---|---|---|---|
| AUS1483 | 446 | GcAGaggtgaagcgaA+G+TGC | 5-10-5 |
| AUS1488 | 451 | GcAGaggtgAagcgAA+G+TGC | 4-5-1-4-6 |
| AUS1464 | 427 | GcAGaggtgAagcgaA+G+TGC | 4-5-1-5-5 |

*Motifs show the regions of each sequence from 5′ to 3′ (e.g., W1-G1-W2 or W1-G1-S1-G2-W2), where the separator sequence is shown in a bolded font.

TABLE 21B
Reduction of HBsAg following treatment with modified
multi-segmented antisense oligonucleotides

| AUS No. | | | AUS1483 | AUS1488 | AUS1464 |
|---|---|---|---|---|---|
| SEQ ID NO: | | | 446 | 451 | 427 |
| Days | 3 | mean | −0.44 | −0.49 | −0.40 |
| post- | | SD | 0.29 | 0.24 | 0.06 |
| dosing | 7 | mean | −0.26 | −0.73 | −0.90 |
| | | SD | 0.05 | 0.26 | 0.28 |
| | 10 | mean | −0.31 | −0.88 | −1.09 |
| | | SD | 0.07 | 0.18 | 0.13 |
| | 14 | mean | −0.24 | −0.75 | −0.89 |
| | | SD | 0.12 | 0.45 | 0.09 |
| | 21 | mean | −0.13 | −0.57 | −0.60 |
| | | SD | 0.00 | 0.27 | 0.10 |

| | | | |
|---|---|---|---|
| Max Reduction | −0.44 | −0.88 | −1.09 |

The maximum log 10 serum HBsAg reduction was −0.44±0.07 for gapmer AUS1483, −1.09±0.13 for AUS1464, −0.88±0.18 for AUS1488. The body weight change was comparable with the saline group for all treatment groups.

### Example 22: Antiviral Effect of Modified Multi-Segmented Antisense Oligonucleotides in pAAV-1.2HBV-GTA HDI-HBV Mice

This study evaluated the effect of modified multi-segmented antisense oligonucleotides on the serum levels of HBsAg in HDI-HBV GTA carrier mice. The modified multi-segmented antisense oligonucleotides used in this study was AUS1489; the sequence and motif is provided in Table 22A, below. Each compound was dosed once at 40 mg/kg by subcutaneous injection (SC) to the mice. Normal saline was administered as negative control. AUS1482 was administered as a gapmer reference compound.

Male C57BL/6 mice were purchased from Shanghai SLAC Laboratory Animal CO., Ltd (China). The HDI-HBV mouse model was constructed by hydrodynamic-injection a pAAV-1.2HBV-GTA plasmid containing 1.2-fold full-length HBV sequence. HBsAg-positive mice were used Day 18 after the injection. The mice were maintained under specific pathogen-free conditions in the BSL-2+ animal facility of Hagnzhou Eyoung biotechnology Co., Ltd (China). The methods and purposes of animal experiments are in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 4.0 mg/ml.

Male HDI-HBV GTD carrier mice were randomly divided into each group of 2 to 3 mice. Each group was dosed with one compound at 40 mg/kg and a dose volume of 10 ml/kg of body weight or an equal volume of normal saline as control. HBsAg-positive mice were dosed 30 days after HDI modeling at the animal laboratory of Eyoung via SC.

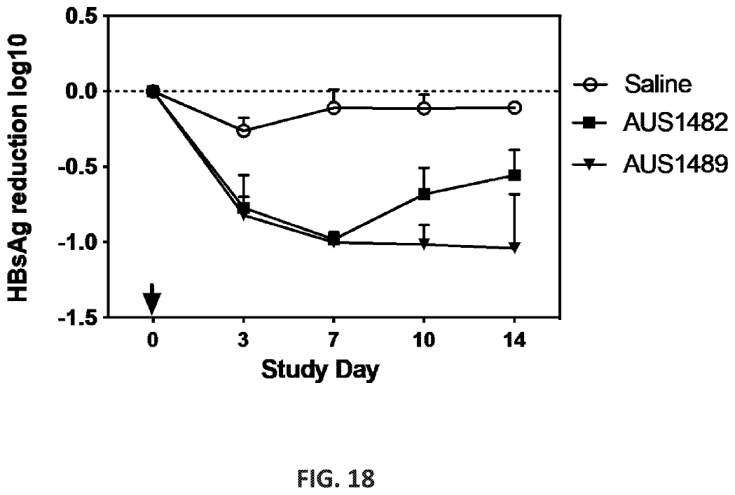

Approximately 80 μl of blood sample was taken from a thigh vein on 0 (before dosing), 3, 7, 10, 14 and 21 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis. Serum HBsAg levels were measured as described in Example 17, above. Results are provided in FIG. 18.

TABLE 22A
Oligonucleotides tested for antisense inhibition of HBV in mouse

| AUS No | SEQ ID NO: | Sequence | Motif* |
|---|---|---|---|
| AUS1482 | 445 | GCAGaggtgaagcgaA+G+TGC | 4-11-5 |
| AUS1489 | 452 | GCAGaggtgAagcgAA+G+TGC | 4-5-1-4-6 |

*Motifs show the regions of each sequence from 5′ to 3′ (e.g., W1-G1-W2 or W1-G1-S1-G2-W2), where the separator sequence is shown in a bolded font.

The maximum log 10 serum HBsAg reduction was more sustainable for the modified multi-segmented antisense oligonucleotides AUS1489 than the control gapmer.

### Example 23: Antiviral Effect of Modified Multi-Segmented Antisense Oligonucleotides in pcDNA3.1-preS2-GTD HDI-HBV Mice

This study evaluated the effect of modified multi-segmented antisense oligonucleotides on the serum levels of HBsAg in HDI-HBV GTD carrier mice. The modified multi-segmented antisense oligonucleotides used in this study were AUS1698, AUS1699 and AUS1700; the sequences and motifs of these modified multi-segmented antisense oligonucleotides are provided in Table 23A, below. Each compound was dosed once at 40 mg/kg by subcutaneous injection (SC) to the mice. Normal saline was administered as negative control. AUS1696 was administered as a gapmer reference compound.

Male C57BL/6 mice were purchased from Shanghai SLAC Laboratory Animal CO., Ltd (China). The HDI-HBV mouse model was constructed by hydrodynamic-injection a pcDNA3.1-preS2-GTD plasmid containing preS2 of HBV sequence. HBsAg-positive mice were used Day 78 after the injection. The mice were maintained under specific pathogen-free conditions in the BSL-2+ animal facility of Hagnzhou Eyoung biotechnology Co., Ltd (China). The methods and purposes of animal experiments are in accordance with the ethical standards and international practices.

HBsAg ELISA kits were purchased from Autobio Diagnostics (China). Normal saline was purchased from Beijing Sanyao Science & Technology Development (China). ELISA was performed using Chemiluminescence immunoassay analyzer (Instrument model: LUMO) of Autobio. Blood was centrifuged using a Thermo Fisher Scientific™ (USA) preparative ultracentrifuge.

The dosing solutions were prepared by dissolving each compound in saline to make a stock solution at 10 mg/ml, which was diluted with saline again at a target concentration of 4.0 mg/ml.

Male HDI-HBV GTD carrier mice were randomly divided into each group of 2 to 3 mice. Each group was dosed with one compound at 40 mg/kg and a dose volume of 10 ml/kg of body weight or an equal volume of normal saline as control. HBsAg-positive mice were dosed 30 days after HDI modeling at the animal laboratory of Eyoung via SC.

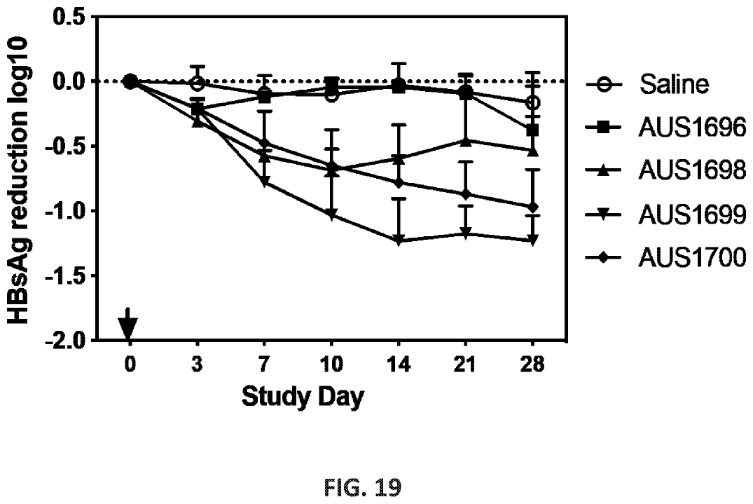

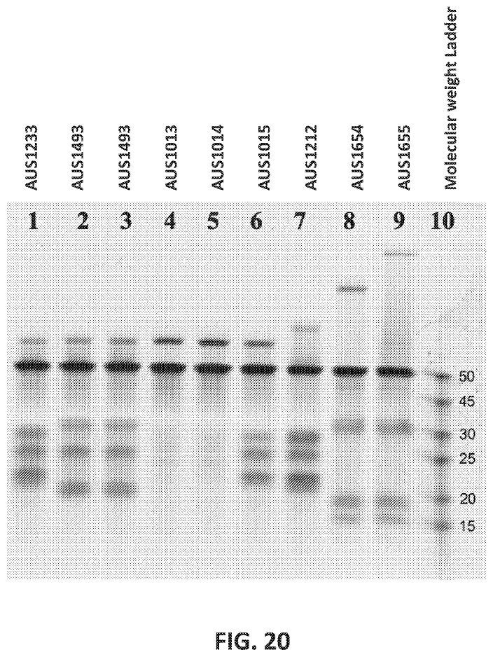

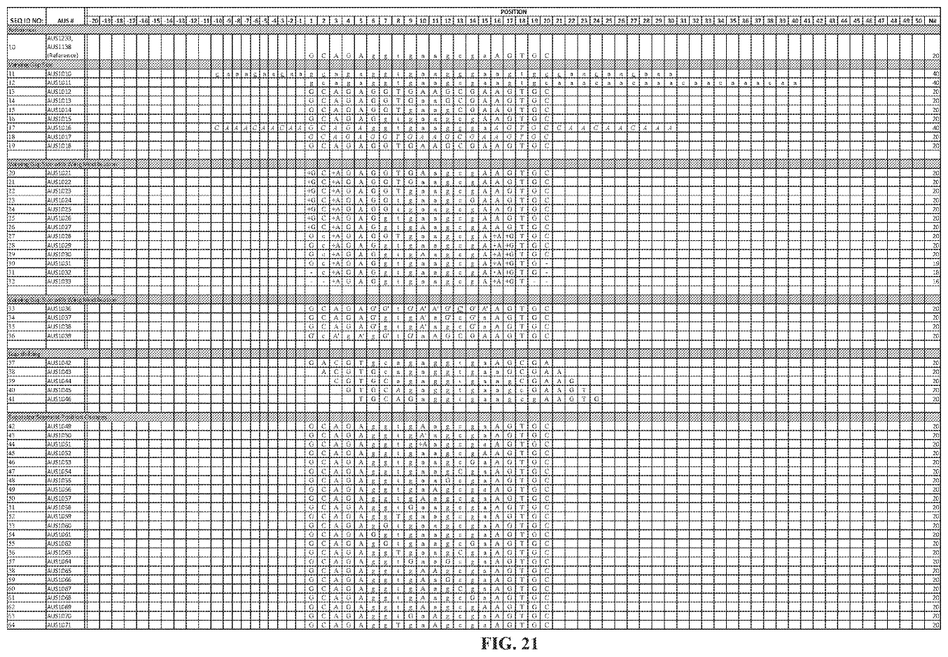

Approximately 80 μl of blood sample was taken from a thigh vein on 0 (before dosing), 3, 7, 10, 14 and 21 days after dosing. Blood samples were placed in an incubator at 37° C. for approximately 30 minutes, and then centrifuged in a precooled (0-4° C.) centrifuge to obtain the serum samples. The obtained serum samples were stored in a −20° C. refrigerator for further analysis. Serum HBsAg levels were measured as described in Example 17, above. Results are provided in FIG. 19, which shows that the antiviral effects of the present modified multi-segmented oligonucleotides AUS1698, AUS1699 and AUS1700 were much better than the corresponding control gapmer AUS1696.

TABLE 23A
Oligonucleotides tested for antisense inhibition of HBV in mouse

| AUS No | SEQ ID NO: | Sequence | Motif* |
|---|---|---|---|
| AUS1696 | 652 | GTGAAgcgaagtgcaCACGG | 5-10-5 |
| AUS1698 | 654 | GTGAAgcgaAgtgcaCACGG | 5-4-1-5-5 |
| AUS1699 | 655 | GTGAAgcgaaGtgcaCACGG | 5-5-1-4-5 |
| AUS1700 | 656 | GTGAAgcGaaGtgcaCACGG | 5-2-1-2-1-4-5 |

*Motifs show the regions of each sequence from 5′ to 3′ (e.g., W1-G1-W2 or W1-G1-S1-G2-W2), where the separator sequence is shown in a bolded font.

TABLE 1A
Inhibition of HBV expression in HepG2.2.15 cells using modified multisegmented antisense oligonucleotides

| | | | | | | | | | |
|---|---|---|---|---|---|---|---|---|---|
| Ausper | SEQ ID | | | | | Fold | Fold | C/E | Tm |
| No. | NO: | Sequence | Motif* | Target | Indication | EC50 | CC30 | ratio | (° C.) |

Combination of Separator position + Wing Mutation results in inhibitory effect

| | | | | | | | | | |
|---|---|---|---|---|---|---|---|---|---|
| AUS1383 | 347 | +G+CA+GaggtgAagcgaAGTGC | 4-5-1-5-5 | HBV | HBV-related disease | 0.13 | 0.97 | 7.39 | |
| AUS1429 | 392 | GCAGaggtgAagcgaAGT+G+C | 4-5-1-5-5 | HBV | HBV-related disease | 0.08 | 1.22 | 15.74 | |
| AUS1431 | 394 | GCAGaggtgAagcgaAGT+G+C | 4-5-1-5-5 | HBV | HBV-related disease | 0.05 | 1.04 | 19.01 | |
| AUS1436 | 399 | +GC+AGaggtgAagcgaAGTGC | 4-5-1-5-5 | HBV | HBV-related disease | 0.24 | 1.11 | 4.65 | |
| AUS1437 | 400 | +GC+AGaggtgAagcgaAGTGC | 4-5-1-5-5 | HBV | HBV-related disease | 0.37 | 1.33 | 3.63 | |
| AUS1441 | 404 | +Gc+AGaggtgAagcgaAGTGC | 4-5-1-5-5 | HBV | HBV-related disease | 0.70 | 1.31 | 1.85 | |
| AUS1463 | 426 | GCAGaggtgAagcgaA+G+TGC | 4-5-1-5-5 | HBV | HBV-related disease | 0.27 | 1.74 | 6.37 | |
| AUS1464 | 427 | GcAGaggtgAagcgaA+G+TGC | 4-5-1-5-5 | HBV | HBV-related disease | 0.38 | 2.10 | 5.45 | |
| AUS1466 | 429 | GCAGaggtgAagcgaA+GTG+C | 4-5-1-5-5 | HBV | HBV-related disease | 0.19 | 1.20 | 6.15 | |
| AUS1467 | 430 | GcAGaggtgAagcgaA+GTG+C | 4-5-1-5-5 | HBV | HBV-related disease | 0.24 | 1.14 | 4.72 | |
| AUS1468 | 431 | GCAGaggtgAagcgaA+GT+GC | 4-5-1-5-5 | HBV | HBV-related disease | 0.25 | 1.30 | 5.29 | |
| AUS1470 | 433 | GCAGaggtgAagcgaA+G+T+GC | 4-5-1-5-5 | HBV | HBV-related disease | 0.16 | 1.16 | 7.09 | |
| AUS1471 | 434 | GcAGaggtgAagcgaA+G+T+GC | 4-5-1-5-5 | HBV | HBV-related disease | 0.18 | 1.23 | 7.03 | |
| AUS1476 | 439 | GCAGaggtgAagcgaA+GT+G+C | 4-5-1-5-5 | HBV | HBV-related disease | 0.10 | 1.49 | 15.50 | |
| AUS1477 | 440 | GcAGaggtgAagcgaA+GT+G+C | 4-5-1-5-5 | HBV | HBV-related disease | 0.20 | 1.28 | 6.24 | |
| AUS1484 | 447 | GCAGaggtgAagcgaAGTGC | 4-5-1-5-5 | HBV | HBV-related disease | 0.47 | 1.24 | 2.65 | |
| AUS1485 | 448 | GcAGaggtgAagcgaAGTGC | 4-5-1-5-5 | HBV | HBV-related disease | 0.54 | 1.24 | 2.29 | |
| AUS1492 | 455 | GCAGaggtgAagcgaA+G+TGC | 4-5-1-5-5 | HBV | HBV-related disease | 0.25 | 1.33 | 5.32 | |
| AUS1493 | 456 | GCAGaggtgAagcgaA+GT+G+C | 4-5-1-5-5 | HBV | HBV-related disease | 0.38 | 1.63 | 4.25 | |
| AUS1494 | 457 | GCAGaggtgAagcga+AGT+GC | 4-5-1-5-5 | HBV | HBV-related disease | 0.22 | 1.47 | 6.59 | |
| AUS1495 | 458 | GCAGaggtgAagcgaA+GTG+C | 4-5-1-5-5 | HBV | HBV-related disease | 0.42 | 1.45 | 3.42 | |
| AUS1502 | 465 | GCAGaggtgAagcgaA+GT+G+C | 4-5-1-5-5 | HBV | HBV-related disease | | | | |
| AUS1382 | 346 | +G+CA+GAggtgAagcgaaGTGC | 5-4-1-6-4 | HBV | HBV-related disease | 0.18 | 1.06 | 5.99 | |
| AUS1384 | 348 | +G+CA+GaggtgAagcgaaGTGC | 4-5-1-6-4 | HBV | HBV-related disease | 0.15 | 1.03 | 6.86 | |
| AUS1385 | 349 | +GC+A+GAggtgAagcgaaGTGC | 5-4-1-6-4 | HBV | HBV-related disease | 0.23 | 0.90 | 3.98 | |
| AUS1387 | 350 | +GC+AGAggtgAagcgaAGUGC | 5-4-1-5-5 | HBV | HBV-related disease | 1.29 | 0.82 | 0.64 | |
| AUS1388 | 351 | +GC+AGAggtgAagcgaaGUGC | 5-4-1-6-4 | HBV | HBV-related disease | 0.09 | 0.92 | 9.72 | |
| AUS1389 | 352 | +GC+AGAggtgAagcgaagU+G+C | 5-4-1-7-3 | HBV | HBV-related disease | 0.13 | 0.71 | 5.31 | |
| AUS1390 | 353 | +GC+AGAggtgAagcgaaGUGC | 5-4-1-6-4 | HBV | HBV-related disease | 0.15 | 0.76 | 4.92 | |
| AUS1391 | 354 | +GC+AGAggtgAagcgaagUGC | 5-4-1-7-3 | HBV | HBV-related disease | 0.19 | 0.76 | 3.96 | |
| AUS1392 | 355 | +GC+AGAggtgAagcgaagUG+C | 5-4-1-7-3 | HBV | HBV-related disease | 0.25 | 0.71 | 2.81 | |
| AUS1393 | 356 | +GC+AGAggtgAagcgaagU+G+C | 5-4-1-7-3 | HBV | HBV-related disease | 0.27 | 0.70 | 2.59 | |
| AUS1396 | 359 | +G+CAGAggtgAagcgaaGUGC | 5-4-1-6-4 | HBV | HBV-related disease | 0.13 | 0.95 | 7.48 | |
| AUS1397 | 360 | +G+CAGAggtgAagcgaagUGC | 5-4-1-7-3 | HBV | HBV-related disease | | | | |
| AUS1398 | 361 | +G+CAGAggtgAagcgaagUG+C | 5-4-1-7-3 | HBV | HBV-related disease | | | | |
| AUS1399 | 362 | +G+CAGAggtgAagcgaagU+G+C | 5-4-1-7-3 | HBV | HBV-related disease | | | | |
| AUS1401 | 364 | GCAGAggtgAagcgaagU+G+C | 5-4-1-7-3 | HBV | HBV-related disease | | | | |
| AUS1402 | 365 | Gc+AGAggtgAagcgaagU+G+C | 5-4-1-7-3 | HBV | HBV-related disease | | | | |
| AUS1403 | 366 | +Gc+AGAggtgAagcgaaGUGC | 5-4-1-6-4 | HBV | HBV-related disease | | | | |
| AUS1404 | 367 | +Gc+AGAggtgAagcgaagUGC | 5-4-1-7-3 | HBV | HBV-related disease | | | | |
| AUS1405 | 368 | +Gc+AGAggtgAagcgaagUG+C | 5-4-1-7-3 | HBV | HBV-related disease | | | | |
| AUS1406 | 369 | +Gc+AGAggtgAagcgaagU+G+C | 5-4-1-7-3 | HBV | HBV-related disease | | | | |
| AUS1407 | 370 | Gc+A+GAggtgAagcgaagU+G+C | 5-4-1-7-3 | HBV | HBV-related disease | | | | |
| AUS1408 | 371 | G+C+AGAggtgAagcgaagU+G+C | 5-4-1-7-3 | HBV | HBV-related disease | | | | |
| AUS1409 | 372 | +G+CA+GAggtgAagcgaaGUGC | 5-4-1-6-4 | HBV | HBV-related disease | 0.04 | 1.61 | 38.05 | |
| AUS1410 | 373 | +G+CA+GAggtgAagcgaagUGC | 5-4-1-7-3 | HBV | HBV-related disease | 0.07 | 1.25 | 17.88 | |
| AUS1411 | 374 | +G+CA+GAggtgAagcgaagUG+C | 5-4-1-7-3 | HBV | HBV-related disease | 0.08 | 0.83 | 10.18 | |
| AUS1413 | 376 | +G+CA+GAggtgAagcgaagUGC | 5-4-1-7-3 | HBV | HBV-related disease | 0.07 | 0.84 | 12.46 | |
| AUS1414 | 377 | +G+CA+GAggtgAagcgaagUG+C | 5-4-1-7-3 | HBV | HBV-related disease | 0.07 | 0.72 | 10.98 | |
| AUS1415 | 378 | +Gc+A+GAggtgAagcgaaGUGC | 5-4-1-6-4 | HBV | HBV-related disease | 0.06 | 0.75 | 13.29 | |
| AUS1416 | 379 | +Gc+A+GAggtgAagcgaagUGC | 5-4-1-7-3 | HBV | HBV-related disease | | | | |
| AUS1422 | 385 | GCAGAggtgAagcgaAGT+G+C | 5-4-1-5-5 | HBV | HBV-related disease | 0.10 | 0.99 | 10.07 | |
| AUS1423 | 386 | GCAGAggtgAagcgaAG+TG+C | 5-4-1-5-5 | HBV | HBV-related disease | 0.07 | 0.99 | 13.47 | |
| AUS1424 | 387 | GCAGAggtgAagcgaA+GTG+C | 5-4-1-5-5 | HBV | HBV-related disease | 0.08 | 0.97 | 12.62 | |
| AUS1425 | 388 | GCAGAggtgAagcgaAG+T+GC | 5-4-1-5-5 | HBV | HBV-related disease | 0.10 | 0.95 | 9.21 | |
| AUS1426 | 389 | GCAGAggtgAagcgaA+GT+GC | 5-4-1-5-5 | HBV | HBV-related disease | 0.08 | 0.83 | 10.02 | |
| AUS1427 | 390 | GCAGAggtgAagcgaA+G+TGC | 5-4-1-5-5 | HBV | HBV-related disease | 0.07 | 0.77 | 11.07 | |
| AUS1428 | 391 | GCAGAggtgAagcgaAGT+G+C | 5-4-1-5-5 | HBV | HBV-related disease | 0.08 | 0.92 | 11.95 | |
| AUS1430 | 393 | GCAGAggtgAagcgaaGT+G+C | 5-4-1-6-4 | HBV | HBV-related disease | 0.07 | 1.19 | 17.15 | |
| AUS1432 | 395 | GCAGAggtgAagcgaaGT+G+C | 5-4-1-6-4 | HBV | HBV-related disease | 0.05 | 1.19 | 23.71 | |
| AUS1433 | 396 | +Gc+AGaggtgAagcgaaGTGC | 4-5-1-6-4 | HBV | HBV-related disease | 0.10 | 1.09 | 10.92 | |
| AUS1434 | 397 | +GC+AGAggtgAagcgaAGTGC | 5-4-1-5-5 | HBV | HBV-related disease | 0.76 | 1.15 | 1.52 | |
| AUS1435 | 398 | +GC+AGAggtgAagcgaAGTGC | 5-4-1-5-5 | HBV | HBV-related disease | 0.18 | 1.22 | 6.70 | |
| AUS1438 | 401 | +GC+AGAggtgAagcgaaGTGC | 5-4-1-6-4 | HBV | HBV-related disease | 0.15 | 0.89 | 5.76 | |
| AUS1439 | 402 | +Gc+AGAggtgAagcgaAGTGC | 5-4-1-5-5 | HBV | HBV-related disease | 0.34 | 1.25 | 3.72 | |
| AUS1440 | 403 | +Gc+AGAggtgAagcgaaGTGC | 5-4-1-6-4 | HBV | HBV-related disease | 0.18 | 1.23 | 6.75 | |
| AUS1443 | 406 | +GC+AgAggtgAagcgaAGTGC | 5-4-1-5-5 | HBV | HBV-related disease | 0.18 | 0.96 | 5.25 | |
| AUS1444 | 407 | +Gc+AgAggtgAagcgaAGTGC | 5-4-1-5-5 | HBV | HBV-related disease | 0.22 | 0.93 | 4.17 | |
| AUS1445 | 408 | +GC+AgaggtgAagcgaAGTGC | 3-6-1-5-5 | HBV | HBV-related disease | 0.25 | 1.01 | 4.03 | |
| AUS1446 | 409 | GCAGAggtgAagcgAAGTGC | 5-4-1-4-6 | HBV | HBV-related disease | 0.40 | 1.46 | 3.67 | |
| AUS1447 | 410 | GCAGaggtgAagcgAAGTGC | 4-5-1-4-6 | HBV | HBV-related disease | 0.52 | 1.11 | 2.12 | |
| AUS1448 | 411 | GcAGAggtgAagcgAAGTGC | 5-4-1-4-6 | HBV | HBV-related disease | 0.67 | 1.07 | 1.59 | |
| AUS1449 | 412 | GCAgAggtgAagcgAAGTGC | 5-4-1-4-6 | HBV | HBV-related disease | 0.74 | 1.04 | 1.41 | |
| AUS1450 | 413 | GCaGAggtgAagcgAAGTGC | 5-4-1-4-6 | HBV | HBV-related disease | 0.96 | 1.26 | 1.32 | |
| AUS1451 | 414 | GCAGAggtgAagcgaAGTGC | 5-4-1-5-5 | HBV | HBV-related disease | 0.70 | 1.22 | 1.75 | |
| AUS1452 | 415 | GcAGAggtgAagcgaAGTGC | 5-4-1-5-5 | HBV | HBV-related disease | 0.54 | 1.09 | 2.00 | |
| AUS1453 | 416 | GCAgAggtgAagcgaAGTGC | 5-4-1-5-5 | HBV | HBV-related disease | 0.84 | 1.22 | 1.46 | |
| AUS1454 | 417 | GCAGAggtgAagcgAAGTGC | 5-4-1-4-6 | HBV | HBV-related disease | 0.65 | 1.00 | 1.52 | |
| AUS1455 | 418 | GCAGAggtgAagcgaaGTGC | 5-4-1-6-4 | HBV | HBV-related disease | 0.59 | 1.06 | 1.80 | |
| AUS1456 | 419 | GCAGaggtgAagcgaaGTGC | 4-5-1-6-4 | HBV | HBV-related disease | 0.56 | 1.08 | 1.91 | |
| AUS1457 | 420 | GCAgaggtgAagcgaaGTGC | 3-6-1-6-4 | HBV | HBV-related disease | 0.53 | 1.15 | 2.15 | |
| AUS1458 | 421 | GCAGAggtgAagcga+AGTG+C | 5-4-1-5-5 | HBV | HBV-related disease | 0.22 | 0.95 | 4.38 | |
| AUS1459 | 422 | GCAGAggtgAagcga+AGT+GC | 5-4-1-5-5 | HBV | HBV-related disease | 0.25 | 1.08 | 4.33 | |
| AUS1460 | 423 | GCAGAggtgAagcga+AG+TGC | 5-4-1-5-5 | HBV | HBV-related disease | 0.23 | 1.29 | 5.71 | |
| AUS1461 | 424 | GCAGAggtgAagcgaA+G+TGC | 5-4-1-5-5 | HBV | HBV-related disease | 0.40 | 1.75 | 4.41 | |
| AUS1462 | 425 | GcAGAggtgAagcgaA+G+TGC | 5-4-1-5-5 | HBV | HBV-related disease | 0.25 | 1.77 | 7.14 | |
| AUS1465 | 428 | GcAGAggtgAagcgaA+GTG+C | 5-4-1-5-5 | HBV | HBV-related disease | 0.43 | 1.32 | 3.03 | |
| AUS1469 | 432 | GcAGAggtgAagcgaA+G+T+GC | 5-4-1-5-5 | HBV | HBV-related disease | 0.25 | 1.22 | 4.90 | |
| AUS1472 | 435 | GCAgAggtgAagcgaA+G+TGC | 5-4-1-5-5 | HBV | HBV-related disease | 0.19 | 1.31 | 7.01 | |
| AUS1473 | 436 | GCaGAggtgAagcgaA+G+TGC | 5-4-1-5-5 | HBV | HBV-related disease | 0.14 | 1.37 | 9.49 | |
| AUS1474 | 437 | GcAgAggtgAagcgaA+G+TGC | 5-4-1-5-5 | HBV | HBV-related disease | 0.16 | 1.42 | 9.05 | |
| AUS1475 | 438 | GcAGAggtgAagcgaA+GT+G+C | 5-4-1-5-5 | HBV | HBV-related disease | 0.18 | 1.31 | 7.39 | |
| AUS1478 | 441 | GcaGAggtgAagcgaA+G+TGC | 5-4-1-5-5 | HBV | HBV-related disease | 0.37 | 1.33 | 3.62 | |
| AUS1479 | 442 | GCagAggtgAagcgaA+G+TGC | 5-4-1-5-5 | HBV | HBV-related disease | 0.13 | 1.56 | 12.00 | |
| AUS1486 | 449 | GCaGAggtgAagcgaAGTGC | 5-4-1-5-5 | HBV | HBV-related disease | 0.76 | 1.46 | 1.92 | |
| AUS1487 | 450 | GcAgAggtgAagcgaAGTGC | 5-4-1-5-5 | HBV | HBV-related disease | 0.68 | 1.63 | 2.39 | |
| AUS1488 | 451 | GcAGaggtgAagcgAA+G+TGC | 4-5-1-4-6 | HBV | HBV-related disease | 0.20 | 1.73 | 8.62 | |
| AUS1489 | 452 | GCAGaggtgAagcgAA+G+TGC | 4-5-1-4-6 | HBV | HBV-related disease | 0.18 | 1.81 | 10.33 | |
| AUS1490 | 453 | GCAGAggtgAagcgAA+G+TGC | 5-4-1-4-6 | HBV | HBV-related disease | 0.16 | 1.77 | 11.26 | 73 |
| AUS1496 | 459 | GcAGAggtgAagcgaA+G+TGC | 5-4-1-5-5 | HBV | HBV-related disease | | | | |
| AUS1497 | 460 | GcAGaggtgAagcgAA+G+TGC | 4-5-1-4-6 | HBV | HBV-related disease | | | | |
| AUS1498 | 461 | GcAGAggtgAagcgAA+G+TGC | 5-4-1-4-6 | HBV | HBV-related disease | | | | |
| AUS1499 | 462 | GcAGAggtgAagcgaA+GTG+C | 5-4-1-5-5 | HBV | HBV-related disease | | | | |
| AUS1500 | 463 | GcAGAggtgAagcga+AGT+GC | 5-4-1-5-5 | HBV | HBV-related disease | | | | |
| AUS1501 | 464 | GCAgAggtgAagcgaAGTGC | 5-4-1-5-5 | HBV | HBV-related disease | | | | |
| AUS1418 | 381 | GTCtgtgcatctcTCC | 3-10-3 | HBV | HBV-related disease | | | | |
| AUS1419 | 382 | +G+T+Ctgtgcatctc+T+C+C | 3-10-3 | HBV | HBV-related disease | | | | |
| AUS1420 | 383 | GCtcatactcgtaggcCA | 2-14-2 | HBV | HBV-related disease | | | | |
| AUS1421 | 384 | +G+Ctcatactcgtaggc+C+A | 2-14-2 | HBV | HBV-related disease | | | | |
| AUS1442 | 405 | GCAGAggtgaagcgaAGTGC | 5-10-5 | HBV | HBV-related disease | | | | |
| AUS1480 | 443 | GCAGAggtgaagcgaA+G+TGC | 5-10-5 | HBV | HBV-related disease | 0.14 | 1.27 | 8.90 | 76 |
| AUS1481 | 444 | GcAGAggtgaagcgaA+G+TGC | 5-10-5 | HBV | HBV-related disease | 0.14 | 1.05 | 7.43 | 75 |
| AUS1482 | 445 | GCAGaggtgaagcgaA+G+TGC | 4-11-5 | HBV | HBV-related disease | 0.14 | 0.90 | 6.63 | |
| AUS1483 | 446 | GcAGaggtgaagcgaA+G+TGC | 4-11-5 | HBV | HBV-related disease | 0.14 | 1.07 | 7.62 | |
| AUS1491 | 454 | +G+T+Ctgtgcatctc+T+C+C | 3-10-3 | HBV | HBV-related disease | | | | |
| AUS1394 | 357 | +GC+AGAggtgAagcgaagtGC | 5-4-1-8-2 | HBV | HBV-related disease | 0.24 | 0.87 | 3.58 | |
| AUS1395 | 358 | +GC+AGAggtgAagcgaagt+G+C | 5-4-1-8-2 | HBV | HBV-related disease | 0.17 | 0.97 | 5.76 | |
| AUS1400 | 363 | +G+CAGAggtgAagcgaagt+G+C | 5-4-1-8-2 | HBV | HBV-related disease | | | | |
| AUS1412 | 375 | +G+CA+GAggtgAagcgaagtG+C | 5-4-1-8-2 | HBV | HBV-related disease | 0.06 | 0.88 | 14.00 | |
| AUS1417 | 380 | +Gc+A+GAggtgAagcgaagtG+C | 5-4-1-8-2 | HBV | HBV-related disease | | | | |

Varying Gap Size with Wing Modification

| | | | | | |
|---|---|---|---|---|---|
| AUS1027 | 26 | +GC+AGAGgtgAagcgAAGTGC | 6-3-1-4-6 | HBV | HBV-related disease |
| AUS1030 | 29 | Gc+AGAGgtgAagcgA+A+GTGC | 6-3-1-4-6 | HBV | HBV-related disease |
| AUS1021 | 20 | +GC+AGAGGTGAagcgAAGTGC | 10-4-6 | HBV | HBV-related disease |
| AUS1022 | 21 | +GC+AGAGGTGaagcgAAGTGC | 9-5-6 | HBV | HBV-related disease |
| AUS1023 | 22 | +GC+AGAGGTgaagcgAAGTGC | 8-6-6 | HBV | HBV-related disease |
| AUS1024 | 23 | +GC+AGAGGtgaagcGAAGTGC | 7-6-7 | HBV | HBV-related disease |
| AUS1025 | 24 | +GC+AGAGGtgaagcgAAGTGC | 7-7-6 | HBV | HBV-related disease |
| AUS1026 | 25 | +GC+AGAGgtgaagcgAAGTGC | 6-8-6 | HBV | HBV-related disease |
| AUS1028 | 27 | Gc+AGAGGTgaagcgA+A+GTGC | 8-6-6 | HBV | HBV-related disease |
| AUS1029 | 28 | Gc+AGAGgtgaagcgA+A+GTGC | 6-8-6 | HBV | HBV-related disease |
| AUS1031 | 30 | Gc+AGAGgtgaagcgA+A+GTG | 6-8-5 | HBV | HBV-related disease |
| AUS1032 | 31 | c+AGAGgtgaagcgA+A+GTG | 5-8-5 | HBV | HBV-related disease |
| AUS1033 | 32 | +AGAGgtgaagcgA+A+GT | 4-8-4 | HBV | HBV-related disease |

Gap Modification

| | | | | | | | | |
|---|---|---|---|---|---|---|---|---|
| AUS1038 | 35 | GCAGAG′gtgA′agcG′aAGTGC | 6-3-1-3-7 | HBV | HBV-related disease | 1.53 | 1.39 | 0.91 |
| AUS1037 | 34 | GCAGAG′gtgA′aG′cG′aAGTGC | 6-3-1-1-1- | HBV | HBV-related disease | 1.69 | 2.23 | 1.32 |
| | | | 1-7 | | | | | |

Gap Modification: Gap shifting

| | | | | | | | | |
|---|---|---|---|---|---|---|---|---|
| AUS1042 | 37 | GACGTgcagaggtgaAGCGA | 5-10-5 | HBV | HBV-related disease | 2.38 | 1.57 | 0.66 |
| AUS1043 | 38 | ACGTGcagaggtgaaGCGAA | 5-10-5 | HBV | HBV-related disease | 1.41 | 1.51 | 1.07 |
| AUS1044 | 39 | CGTGCagaggtgaagCGAAG | 5-10-5 | HBV | HBV-related disease | 2.47 | 1.09 | 0.44 |
| AUS1045 | 40 | GTGCAgaggtgaagcGAAGT | 5-10-5 | HBV | HBV-related disease | 3.09 | 1.49 | 0.48 |
| AUS1046 | 41 | TGCAGaggtgaagcgAAGTG | 5-10-5 | HBV | HBV-related disease | 3.65 | 2.13 | 0.58 |

Gap Modification, Separator position variance

| | | | | | | | | | |
|---|---|---|---|---|---|---|---|---|---|
| AUS1049 | 42 | GCAGAggtgAagcgaAGTGC | 5-4-1-5-5 | HBV | HBV-related disease | 1.42 | 2.11 | 1.49 | 69.5 |
| AUS1050 | 43 | GCAGAggtgA′agcgaAGTGC | 5-4-1-5-5 | HBV | HBV-related disease | 0.90 | 1.65 | 1.83 | 71 |
| AUS1051 | 44 | GCAGAggtg+AagcgaAGTGC | 5-4-1-5-5 | HBV | HBV-related disease | 1.66 | 2.14 | 1.30 | 73 |
| AUS1054 | 47 | GCAGAggtgaagCgaAGTGC | 5-7-1-2-5 | HBV | HBV-related disease | 2.16 | 0.89 | 0.41 | 69 |
| AUS1055 | 48 | GCAGAggtgaaGcgaAGTGC | 5-6-1-3-5 | HBV | HBV-related disease | 2.25 | 0.54 | 0.24 | 68.5 |
| AUS1056 | 49 | GCAGAggtgaAgcgaAGTGC | 5-5-1-4-5 | HBV | HBV-related disease | 2.32 | 1.51 | 0.65 | 70 |
| AUS1057 | 50 | GCAGAggtgAagcgaAGTGC | 5-4-1-5-5 | HBV | HBV-related disease | 1.33 | 0.80 | 0.60 | 69.5 |
| AUS1058 | 51 | GCAGAggtGaagcgaAGTGC | 5-3-1-6-5 | HBV | HBV-related disease | 1.64 | 0.95 | 0.58 | 70 |
| AUS1059 | 52 | GCAGAggTgaagcgaAGTGC | 5-2-1-7-5 | HBV | HBV-related disease | 2.14 | 0.75 | 0.35 | 70.5 |
| AUS1069 | 62 | GCAGAggtgAagcgAAGTGC | 5-4-1-4-6 | HBV | HBV-related disease | 1.72 | 1.11 | 0.64 | 69.5 |
| AUS1073 | 66 | GCAGAGgtgaAgcgaAGTGC | 6-4-1-4-5 | HBV | HBV-related disease | 1.65 | 1.20 | 0.72 | |
| AUS1078 | 71 | GCAGAG″gtgaagcG″aAGTGC | 6-7-1-1-5 | HBV | HBV-related disease | 2.66 | 1.26 | 0.48 | |
| AUS1065 | 58 | GCAGAggtgAAgcgaAGTGC | 5-4-2-4-5 | HBV | HBV-related disease | 2.50 | 1.66 | 0.66 | 69.5 |
| AUS1064 | 57 | GCAGAggtGaaGcgaAGTGC | 5-3-1-2-1- | HBV | HBV-related disease | 6.19 | 2.45 | 0.40 | 68 |
| | | | 3-5 | | | | | | |
| AUS1066 | 59 | GCAGAggtgAaGcgaAGTGC | 5-4-1-1-1- | HBV | HBV-related disease | 2.50 | 2.28 | 0.91 | 68.5 |
| | | | 3-5 | | | | | | |
| AUS1067 | 60 | GCAGAggtgAagCgaAGTGC | 5-4-1-2-1- | HBV | HBV-related disease | 3.67 | 1.60 | 0.44 | 67.5 |
| | | | 2-5 | | | | | | |
| AUS1070 | 63 | GCAGAggtGaAgcgaAGTGC | 5-3-1-1-1- | HBV | HBV-related disease | 1.90 | 1.43 | 0.75 | 68.5 |
| | | | 4-5 | | | | | | |
| AUS1071 | 64 | GCAGAggTgaAgcgaAGTGC | 5-2-1-2-1- | HBV | HBV-related disease | 2.54 | 2.34 | 0.92 | 68.5 |
| | | | 4-5 | | | | | | |
| AUS1074 | 67 | GCAGAgGtgAagCgaAGTGC | 7-2-1-2-1- | HBV | HBV-related disease | 3.41 | 1.03 | 0.30 | |
| | | | 2-5 | | | | | | |
| AUS1075 | 68 | GCAGAggTgaAgcGaAGTGC | 5-2-1-2-1- | HBV | HBV-related disease | 5.06 | | | |
| | | | 2-7 | | | | | | |
| AUS1052 | 45 | GCAGAggtgaagcgAAGTGC | 5-9-6 | HBV | HBV-related disease | 1.54 | 1.18 | 0.77 | 71 |
| AUS1061 | 54 | GCAGAGgtgaagcgaAGTGC | 6-9-5 | HBV | HBV-related disease | 1.05 | 0.82 | 0.78 | 73 |
| AUS1053 | 46 | GCAGAggtgaagcGaAGTGC | 5-8-1-1-5 | HBV | HBV-related disease | 1.86 | 1.16 | 0.62 | 70 |
| AUS1060 | 53 | GCAGAgGtgaagcgaAGTGC | 5-1-1-8-5 | HBV | HBV-related disease | 2.19 | 1.33 | 0.61 | 71.5 |
| AUS1062 | 55 | GCAGAgGtgaagcGaAGTGC | 5-1-1-6-1- | HBV | HBV-related disease | 2.84 | 2.09 | 0.74 | 70 |
| | | | 1-5 | | | | | | |
| AUS1063 | 56 | GCAGAggTgaagCgaAGTGC | 5-2-1-4-1- | HBV | HBV-related disease | 5.26 | 1.88 | 0.36 | 67.5 |
| | | | 2-5 | | | | | | |
| AUS1068 | 61 | GCAGAggtgAagcGaAGTGC | 5-4-1-3-1- | HBV | HBV-related disease | 2.61 | 1.50 | 0.57 | 68 |
| | | | 1-5 | | | | | | |
| AUS1072 | 65 | GCAGAgGtgaAgcgaAGTGC | 5-1-1-3-1- | HBV | HBV-related disease | 2.80 | 2.36 | 0.84 | 70 |
| | | | 4-5 | | | | | | |
| AUS1076 | 69 | GCAGAggtgAagcgaAGTGCAACAC | 5-4-1-5-25 | HBV | HBV-related disease | 1.55 | | | |
| | | AACACAACACAACAC | | | | | | | |
| AUS1077 | 70 | GCAGAggtgaAgcgaAGTGCAACAC | 5-5-1-4-25 | HBV | HBV-related disease | 1.83 | | | |
| | | AACACAACACAACAC | | | | | | | |
| AUS1079 | 72 | GCAGAggtG″aagcG″aAGTGC | 5-3-1-4-1- | HBV | HBV-related disease | 3.94 | 1.45 | 0.37 | |
| | | | 1-5 | | | | | | |

Different Template Sequence-separator position change affects activity, but effects differ based on position

| | | | | | | | | |
|---|---|---|---|---|---|---|---|---|
| AUS1083 | 75 | GTGAAgcGaagtgcaCACGG | 5-2-1-7-5 | HBV | HBV-related disease | 1.98 | 0.69 | 0.35 |
| AUS1084 | 76 | GTGAAgcgAagtgcaCACGG | 5-3-1-6-5 | HBV | HBV-related disease | 3.71 | >250 | |
| AUS1085 | 77 | GTGAAgcgaAgtgcaCACGG | 5-4-1-5-5 | HBV | HBV-related disease | 2.41 | >250 | |
| AUS1086 | 78 | GTGAAgcgaaGtgcaCACGG | 5-5-1-4-5 | HBV | HBV-related disease | 3.68 | >250 | |
| AUS1087 | 79 | GTGAAgcgaagTgcaCACGG | 5-6-1-3-5 | HBV | HBV-related disease | 5.59 | >250 | |
| AUS1088 | 80 | GTGAAgcgaagtGcaCACGG | 5-7-1-2-5 | HBV | HBV-related disease | 4.93 | >250 | |
| AUS1095 | 87 | GTGAAgcgaAgtgcACACGG | 5-4-1-4-6 | HBV | HBV-related disease | 2.42 | >250 | |
| AUS1099 | 91 | GTGAAGcgaaGtgcaCACGG | 6-4-1-4-5 | HBV | HBV-related disease | 1.55 | >250 | |
| AUS1091 | 83 | GTGAAgcgaAGtgcaCACGG | 5-4-2-4-5 | HBV | HBV-related disease | 2.08 | 0.94 | 0.45 |
| AUS1092 | 84 | GTGAAgcgaAgTgcaCACGG | 5-4-1-1-1- | HBV | HBV-related disease | 9.62 | >250 | |
| | | | 3-5 | | | | | |
| AUS1093 | 85 | GTGAAgcgaAgtGcaCACGG | 5-4-1-2-1- | HBV | HBV-related disease | 7.07 | >250 | |
| | | | 2-5 | | | | | |
| AUS1096 | 88 | GTGAAgcgAaGtgcaCACGG | 5-3-1-1-1- | HBV | HBV-related disease | 1.87 | >250 | |
| | | | 4-5 | | | | | |
| AUS1097 | 89 | GTGAAgcGaaGtgcaCACGG | 5-2-1-2-1- | HBV | HBV-related disease | 1.59 | >250 | |
| | | | 4-5 | | | | | |
| AUS1102 | 94 | GTGAAgCgaAgtGcaCACGG | 7-2-1-2-1- | HBV | HBV-related disease | 17.14 | >250 | |
| | | | 2-5 | | | | | |
| AUS1103 | 95 | GTGAAgcGaaGtgCaCACGG | 5-2-1-2-1- | HBV | HBV-related disease | | | |
| | | | 2-7 | | | | | |
| AUS1081 | 73 | GTGAAGcgaagtgcaCACGG | 6-9-5 | HBV | HBV-related disease | 2.95 | 1.20 | 0.41 |
| AUS1090 | 82 | GTGAAgcgaagtgcACACGG | 5-9-6 | HBV | HBV-related disease | 2.07 | >250 | |
| AUS1082 | 74 | GTGAAgCgaagtgcaCACGG | 5-1-1-8-5 | HBV | HBV-related disease | 2.03 | 0.54 | 0.27 |
| AUS1089 | 81 | GTGAAgcgaagtgCaCACGG | 5-8-1-1-5 | HBV | HBV-related disease | 2.81 | >250 | |
| AUS1094 | 86 | GTGAAgcgaAgtgCaCACGG | 5-4-1-3-1- | HBV | HBV-related disease | 2.83 | >250 | |
| | | | 1-5 | | | | | |
| AUS1098 | 90 | GTGAAgCgaaGtgcaCACGG | 5-1-1-3-1- | HBV | HBV-related disease | 1.70 | >250 | |
| | | | 4-5 | | | | | |
| AUS1100 | 92 | GTGAAGcgaAgtgCaCACGG | 6-3-1-3-1- | HBV | HBV-related disease | 1.75 | >250 | |
| | | | 1-5 | | | | | |
| AUS1101 | 93 | GTGAAgCgaaGtgcACACGG | 5-1-1-3-1- | HBV | HBV-related disease | 5.73 | >250 | |
| | | | 3-6 | | | | | |

Separator position reduced activity and effect differs based on position placement, separator at 10^{th} position has high effect with
different 2′ modifications

| | | | | | | | | | |
|---|---|---|---|---|---|---|---|---|---|
| AUS1175 | 160 | GCAGaggtgAagcgaAGTGC | 4-5-1-5-5 | HBV | HBV-related disease | 3.05 | 1.60 | 0.52 | |
| AUS1128 | 118 | GCAGAggUgaagcgaAGTGC | 5-2-1-7-5 | HBV | HBV-related disease | 1.46 | 0.90 | 0.62 | |
| AUS1129 | 119 | GCAGAggtGaagcgaAGTGC | 5-3-1-6-5 | HBV | HBV-related disease | 1.62 | 1.02 | 0.63 | |
| AUS1130 | 120 | GCAGAggtgAagcgaAGTGC | 5-4-1-5-5 | HBV | HBV-related disease | 1.68 | 0.85 | 0.50 | 70 |
| AUS1131 | 121 | GCAGAggtgaAgcgaAGTGC | 5-5-1-4-5 | HBV | HBV-related disease | 2.06 | 0.92 | 0.45 | 70 |
| AUS1132 | 122 | GCAGAggtgaaGcgaAGTGC | 5-6-1-3-5 | HBV | HBV-related disease | 4.88 | 2.41 | 0.49 | |
| AUS1133 | 123 | GCAGAggtgaagCgaAGTGC | 5-7-1-2-5 | HBV | HBV-related disease | 3.15 | 1.16 | 0.37 | |
| AUS1136 | 126 | GCAGAGgtgaAgcgaAGTGC | 6-4-1-4-5 | HBV | HBV-related disease | 1.54 | 0.87 | 0.57 | |
| AUS1137 | 127 | GCAGAggtgAagcgAAGTGC | 5-4-1-4-6 | HBV | HBV-related disease | 1.63 | 0.80 | 0.49 | |
| AUS1143 | 132 | GCAGAggT″gaagcgaAGUGC | 5-2-1-7-5 | HBV | HBV-related disease | 1.28 | 0.81 | 0.63 | |
| AUS1144 | 133 | GCAGAggtgA″agcgaAGUGC | 5-4-1-5-5 | HBV | HBV-related disease | 2.16 | 0.66 | 0.31 | |
| AUS1145 | 134 | GCAGAggtgaA″gcgaAGUGC | 5-5-1-4-5 | HBV | HBV-related disease | 1.95 | 0.64 | 0.33 | |
| AUS1148 | 137 | GCAGAggtgAagcgaAGTGC | 5-4-1-5-5 | HBV | HBV-related disease | 1.93 | 0.98 | 0.51 | |
| AUS1149 | 138 | GCAGAggtgaAgcgaAGTGC | 5-5-1-4-5 | HBV | HBV-related disease | 1.80 | 0.83 | 0.46 | |
| AUS1151 | 139 | GCAGAGgtgaAgcgaACTGC | 6-4-1-4-5 | HBV | HBV-related disease | 1.11 | 0.82 | 0.74 | 64 |
| AUS1152 | 140 | GCAGAGgtgaAgcgaAGAGC | 6-4-1-4-5 | HBV | HBV-related disease | 2.43 | 1.43 | 0.59 | 66.5 |
| AUS1153 | 141 | GCAGAGgtgaAgcgaAGTCC | 6-4-1-4-5 | HBV | HBV-related disease | 3.35 | 1.58 | 0.47 | 68.5 |
| AUS1154 | 142 | GCAGAGgtgaAgcgaAGTGG | 6-4-1-4-5 | HBV | HBV-related disease | 2.67 | 3.05 | 1.14 | 69.5 |
| AUS1156 | 143 | GCAGAggtgaagCgaAGTGC | 5-7-1-2-5 | HBV | HBV-related disease | 9.5 | 2.7 | 0.29 | |
| AUS1158 | 145 | GCAGAggtgaagCgaAGTGC | 5-7-1-2-5 | HBV | HBV-related disease | 23.1 | 3.2 | 0.14 | |
| AUS1160 | 147 | GCAGAGgtgaAgcgaAGTGC | 6-4-1-4-5 | HBV | HBV-related disease | 15.6 | 2.5 | 0.16 | |
| AUS1168 | 154 | GCAGAggtgaAgcgaAGTGC | 5-5-1-4-5 | HBV | HBV-related disease | 2.90 | 1.17 | 0.40 | |
| AUS1169 | 155 | GCAGAggtgAagcgaAGTGC | 5-4-1-5-5 | HBV | HBV-related disease | 3.98 | 1.26 | 0.32 | |
| AUS1170 | 156 | GCAGAggtgAagcgAAGTGC | 5-4-1-4-6 | HBV | HBV-related disease | 3.51 | 1.25 | 0.35 | |
| AUS1171 | 157 | GCAGAGgtgaAgcgaAGTGC | 6-4-1-4-5 | HBV | HBV-related disease | | | | |
| AUS1173 | 158 | GCAGAggtgAagcgaAGTGC | 5-4-1-5-5 | HBV | HBV-related disease | 0.65 | 0.93 | 1.42 | |
| AUS1174 | 159 | GCAGAggtgAagcgaAGTGC | 5-4-1-5-5 | HBV | HBV-related disease | 1.27 | 1.17 | 0.92 | |
| AUS1176 | 161 | GCAgaggtgAagcgaAGTGC | 3-6-1-5-5 | HBV | HBV-related disease | | | | |
| AUS1178 | 163 | GCAGAggtgAagcgaAGUGC | 5-4-1-5-5 | HBV | HBV-related disease | 1.03 | 0.64 | 0.62 | |
| AUS1181 | 166 | GCAGAggUgaagcgaAGUGC | 5-2-1-7-5 | HBV | HBV-related disease | 1.35 | 0.79 | 0.59 | |
| AUS1182 | 167 | GCAGAggtGaagcgaAGUGC | 5-3-1-6-5 | HBV | HBV-related disease | 2.97 | 0.60 | 0.20 | |
| AUS1183 | 168 | GCAGAggtgaAgcgaAGUGC | 5-5-1-4-5 | HBV | HBV-related disease | 2.52 | 0.64 | 0.25 | |
| AUS1184 | 169 | GCAGAggtgaaGcgaAGUGC | 5-6-1-3-5 | HBV | HBV-related disease | 2.52 | 0.81 | 0.32 | |
| AUS1185 | 170 | GCAGAggtgaagCgaAGUGC | 5-7-1-2-5 | HBV | HBV-related disease | 2.88 | 0.88 | 0.30 | |
| AUS1188 | 173 | GCAGAggtgAagcgAAGUGC | 5-4-1-4-6 | HBV | HBV-related disease | 1.51 | 0.41 | 0.27 | |
| AUS1189 | 174 | GCAGAGgtgaAgcgaAGUGC | 6-4-1-4-5 | HBV | HBV-related disease | 0.94 | 0.64 | 0.68 | |
| AUS1190 | 175 | GCAGAggtgAagcgaAGUGC | 5-4-1-5-5 | HBV | HBV-related disease | 1.34 | 0.91 | 0.68 | |
| AUS1191 | 176 | GCAGAggtgaAgcgaAGUGC | 5-5-1-4-5 | HBV | HBV-related disease | 1.24 | 0.57 | 0.46 | |
| AUS1192 | 177 | GCAGAggtgAagcgAAGUGC | 5-4-1-4-6 | HBV | HBV-related disease | 3.30 | 0.84 | 0.26 | |
| AUS1194 | 179 | GCAGAggtgAagtgaAGUGC | 5-4-1-5-5 | HBV | HBV-related disease | | | | |
| AUS1159 | 146 | GCAGAGgtgaAgCgaAGTGC | 6-4-1-1-1- | HBV | HBV-related disease | 19.6 | 2.1 | 0.11 | |
| | | | 2-5 | | | | | | |
| AUS1161 | 148 | GCAGAGgtgaAgCgaAGTGC | 6-4-1-1-1- | HBV | HBV-related disease | 32.0 | 2.5 | 0.08 | |
| | | | 2-5 | | | | | | |
| AUS1162 | 149 | GCAGAGgtgaAgCga+AG+TG+C | 6-4-1-1-1- | HBV | HBV-related disease | 6.6 | 3.3 | 0.50 | |
| | | | 2-5 | | | | | | |
| AUS1163 | 150 | GCAGAGgtgaAgCga+AG+TG+C | 6-4-1-1-1- | HBV | HBV-related disease | 8.6 | 3.4 | 0.39 | |
| | | | 2-5 | | | | | | |
| AUS1165 | 152 | GC+AGAGgtgaAgCga+AG+TG+C | 6-4-1-1-1- | HBV | HBV-related disease | 9.6 | 3.2 | 0.34 | |
| | | | 2-5 | | | | | | |
| AUS1105 | 96 | GCAGAggtgaagcgaAGTGC | 6-9-5 | HBV | HBV-related disease | 1.24 | 0.88 | 0.71 | |
| AUS1106 | 97 | GCAGAggtgaagcgaAGTGC | 5-1-1-8-5 | HBV | HBV-related disease | 1.80 | 1.06 | 0.59 | |
| AUS1107 | 98 | GCAGAggugaagcgaAGTGC | 5-2-1-7-5 | HBV | HBV-related disease | 2.63 | 1.37 | 0.52 | |
| AUS1108 | 99 | GCAGAggtgaagcgaAGTGC | 5-3-1-6-5 | HBV | HBV-related disease | 1.78 | 0.93 | 0.52 | |
| AUS1109 | 100 | GCAGAggtgaagcgaAGTGC | 5-4-1-5-5 | HBV | HBV-related disease | 1.21 | 0.85 | 0.71 | |
| AUS1110 | 101 | GCAGAggtgaagcgaAGTGC | 5-5-1-4-5 | HBV | HBV-related disease | 2.16 | 1.14 | 0.53 | |
| AUS1111 | 102 | GCAGAggtgaagcgaAGTGC | 5-6-1-3-5 | HBV | HBV-related disease | 2.66 | 1.64 | 0.62 | |
| AUS1112 | 103 | GCAGAggtgaagcgaAGTGC | 5-7-1-2-5 | HBV | HBV-related disease | 2.18 | 1.13 | 0.52 | |
| AUS1113 | 104 | GCAGAggtgaagcgaAGTGC | 5-8-1-1-5 | HBV | HBV-related disease | 1.82 | 1.13 | 0.62 | |
| AUS1114 | 105 | GCAGAggtgaagcgaAGTGC | 5-9-6 | HBV | HBV-related disease | 1.66 | 1.09 | 0.66 | |
| AUS1116 | 106 | GCAGAggagaagcgaAGTGC | 5-10-5 | HBV | HBV-related disease | 2.40 | 1.24 | 0.52 | |
| AUS1117 | 107 | GCAGAggtcaagcgaAGTGC | 5-10-5 | HBV | HBV-related disease | 1.94 | 1.11 | 0.57 | |
| AUS1118 | 108 | GCAGAggtgtagcgaAGTGC | 5-10-5 | HBV | HBV-related disease | 1.93 | 1.31 | 0.68 | |
| AUS1119 | 109 | GCAGAggtgatgcgaAGTGC | 5-10-5 | HBV | HBV-related disease | 1.92 | 0.97 | 0.50 | |
| AUS1120 | 110 | GCAGAggtgaaccgaAGTGC | 5-10-5 | HBV | HBV-related disease | 2.89 | 0.72 | 0.25 | |
| AUS1121 | 111 | GCAGAggtgaagggaAGTGC | 5-10-5 | HBV | HBV-related disease | 3.15 | 3.54 | 1.12 | |
| AUS1122 | 112 | GCAGAggtgaagccaAGTGC | 5-10-5 | HBV | HBV-related disease | 3.12 | 0.54 | 0.17 | |
| AUS1123 | 113 | GCAGAggtgaagcgtAGTGC | 5-10-5 | HBV | HBV-related disease | 2.31 | 0.98 | 0.42 | |
| AUS1124 | 114 | GCAGAggtgaagcgaTGTGC | 5-10-5 | HBV | HBV-related disease | 1.02 | 0.59 | 0.58 | |
| AUS1125 | 115 | GCAGAggtgaagcgaAGTGC | 6-4-1-4-5 | HBV | HBV-related disease | 2.08 | 1.45 | 0.69 | |
| AUS1126 | 116 | GCAGAGgtgaagcgaAGTGC | 6-9-5 | HBV | HBV-related disease | 1.19 | 0.82 | 0.69 | |
| AUS1135 | 125 | GCAGAggtgaagcgAAGTGC | 5-9-6 | HBV | HBV-related disease | 1.68 | 0.90 | 0.54 | |
| AUS1138 | 10 | GCAGAggtgaagcgaAGTGC | 5-10-5 | HBV | HBV-related disease | | | | |
| (Reference) | | | | | | | | | |
| AUS1139 | 128 | GGTCCggcagatgagAAGGC | 5-10-5 | HBV | HBV-related disease | | | | |
| AUS1140 | 129 | CCGGCagatgagaagGCACA | 5-10-5 | HBV | HBV-related disease | | | | |
| AUS1141 | 130 | GGCACagacggggagTCCGC | 5-10-5 | HBV | HBV-related disease | | | | |
| AUS1142 | 131 | GTCCGcgtaaagagaGGTGC | 5-10-5 | HBV | HBV-related disease | | | | |
| AUS1157 | 144 | GCAGAggtgaagcgaAGTGC | 5-10-5 | HBV | HBV-related disease | 10.0 | 2.7 | 0.27 | |
| AUS1177 | 162 | GCAGAggtgaagcgaAGUGC | 5-10-5 | HBV | HBV-related disease | 1.14 | 1.06 | 0.94 | |
| AUS1179 | 164 | GCAGAGgtgaagcgaAGUGC | 6-9-5 | HBV | HBV-related disease | 1.42 | 0.50 | 0.35 | |
| AUS1187 | 172 | GCAGAggtgaagcgAAGUGC | 5-9-6 | HBV | HBV-related disease | 1.11 | 0.76 | 0.68 | |
| AUS1193 | 178 | GCAGAggtgaagtgaAGUGC | 5-10-5 | HBV | HBV-related disease | 4.54 | 1.01 | 0.22 | |
| AUS1127 | 117 | GCAGAgGtgaagcgaAGTGC | 5-1-1-8-5 | HBV | HBV-related disease | 1.43 | 0.76 | 0.53 | |
| AUS1134 | 124 | GCAGAggtgaagcGaAGTGC | 5-8-1-1-5 | HBV | HBV-related disease | 1.55 | 0.89 | 0.57 | |
| AUS1146 | 135 | GCAGAggtgaagcG″aAGUGC | 5-8-1-1-5 | HBV | HBV-related disease | 2.15 | 0.60 | 0.28 | |
| AUS1147 | 136 | GCAGAggtgA″agcG″aAGUGC | 5-4-1-3-1- | HBV | HBV-related disease | 2.14 | 0.59 | 0.27 | |
| | | | 1-5 | | | | | | |
| AUS1164 | 151 | GCAGAggtgAagCgA+AG+TG+C | 5-4-1-2-1- | HBV | HBV-related disease | 5.5 | 2.9 | 0.54 | |
| | | | 1-6 | | | | | | |
| AUS1180 | 165 | GCAGAgGtgaagcgaAGUGC | 5-1-1-8-5 | HBV | HBV-related disease | 1.48 | 0.69 | 0.46 | |
| AUS1186 | 171 | GCAGAggtgaagcGaAGUGC | 5-8-1-1-5 | HBV | HBV-related disease | 1.17 | 0.65 | 0.55 | |
| AUS1168 | 154 | GCAGAggtgaAgcgaAGTGC | 5-5-1-4-5 | HBV | HBV-related disease | 3.63 | 1.11 | 0.31 | |

Varying Wing (cap) Size

| | | | | | | | | |
|---|---|---|---|---|---|---|---|---|
| AUS1016 | 17 | CAAACAACAAGCAGAggtgaagcga | 15-10-15 | HBV | HBV-related disease | | | |
| | | AGTGCCAACAACAAA | | | | | | |
| AUS1198 | 181 | AAACAACAAAGCAGAggtgaagcga | 15-10-15 | HBV | HBV-related disease | 2.2 | >250 | |
| | | AGTGCAAACAACAAA | | | | | | |
| AUS1199 | 182 | AAACAACAAAgcagaggtgaagcga | 10-20-10 | HBV | HBV-related disease | 6.1 | 0.3 | 0.05 |
| | | agtgcAAACAACAAA | | | | | | |
| AUS1200 | 183 | ACAAAGCAGAggtgaagcgaAGTGC | 10-10-10 | HBV | HBV-related disease | 2.7 | >250 | |
| | | AAACA | | | | | | |
| AUS1201 | 184 | ACAAAgcagaggtgaagcgaagtgc | 5-20-5 | HBV | HBV-related disease | 6.1 | 0.2 | 0.04 |
| | | AAACA | | | | | | |
| AUS1202 | 185 | GCAGAggtgaagcgaAGTGCAAACA | 5-10-25 | HBV | HBV-related disease | 0.7 | 0.3 | 0.44 |
| | | ACAAACAACAAACAA | | | | | | |
| AUS1203 | 186 | GCAGAggtgaagcgaAGTGCAAACA | 5-10-15 | HBV | HBV-related disease | 1.8 | >250 | |
| | | ACAAA | | | | | | |
| AUS1204 | 187 | AACAAACAACAAACAACAAAGCAGA | 25-10-5 | HBV | HBV-related disease | 1.0 | 0.3 | 0.32 |
| | | ggtgaagcgaAGTGC | | | | | | |
| AUS1205 | 188 | AAACAACAAAGCAGAggtgaagcga | 15-10-5 | HBV | HBV-related disease | 4.3 | >250 | |
| | | AGTGC | | | | | | |

Varying Wing size (hence total length of antisense oligonucleotide)-At least 18 residues in length provides an effect

| | | | | | | | | | |
|---|---|---|---|---|---|---|---|---|---|
| AUS1206 | 189 | CGTGCAGAggtgaagcgaAGTGCAC | 8-10-8 | HBV | HBV-related disease | 2.16 | 1.71 | 0.79 | 81.4 |
| | | A | | | | | | | |
| AUS1207 | 190 | GTGCAGAggtgaagcgaAGTGCAC | 7-10-7 | HBV | HBV-related disease | 1.20 | 1.68 | 1.41 | |
| AUS1208 | 191 | TGCAGAggtgaagcgaAGTGCA | 6-10-6 | HBV | HBV-related disease | 1.29 | 2.02 | 1.56 | |
| AUS1209 | 192 | CAGAggtgaagcgaAGTG | 4-10-4 | HBV | HBV-related disease | 6.78 | >250 | | 65 |
| AUS1210 | 193 | AGAggtgaagcgaAGT | 3-10-3 | HBV | HBV-related disease | 22.96 | >250 | | |
| AUS1211 | 194 | GAggtgaagcgaAG | 2-10-2 | HBV | HBV-related disease | >250 | >250 | | |
| AUS1212 | 195 | GCAGAggtgaagcgaAGTGCAACAC | 5-10-10 | HBV | HBV-related disease | 2.2 | 0.9 | 0.39 | |
| AUS1213 | 196 | GCAGAggtgaagcgaAGTGCAACAC | 5-10-15 | HBV | HBV-related disease | 1.6 | 0.7 | 0.41 | |
| | | AACAC | | | | | | | |
| AUS1214 | 197 | GCAGAggtgaagcgaAGTGCAACAC | 5-10-20 | HBV | HBV-related disease | 1.3 | 0.6 | 0.50 | |
| | | AACACAACAC | | | | | | | |
| AUS1215 | 198 | GCAGAggtgaagcgaAGTGCAACAC | 5-10-25 | HBV | HBV-related disease | 1.1 | 0.4 | 0.35 | |
| | | AACACAACACAACAC | | | | | | | |
| AUS1216 | 199 | GCAGAggtgaagcgaAGTGCAACAC | 5-10-30 | HBV | HBV-related disease | 1.6 | 0.4 | 0.25 | |
| | | AACACAACACAACACAACAC | | | | | | | |
| AUS1217 | 200 | GCAGAggtgaagcgaAGTGCAACAC | 5-10-35 | HBV | HBV-related disease | 1.8 | 0.3 | 0.16 | |
| | | AACACAACACAACACAACACAACAC | | | | | | | |
| AUS1218 | 201 | GCAGAggtgaagcgaAGTGCAAACA | 5-10-25 | HBV | HBV-related disease | 1.2 | 0.5 | 0.44 | |
| | | ACAAACAACAAACAA | | | | | | | |
| AUS1219 | 202 | GCAGAggtgaagcgaAGTGCACACA | 5-10-25 | HBV | HBV-related disease | 0.9 | 0.3 | 0.30 | |
| | | CACACACACACACAC | | | | | | | |
| AUS1220 | 203 | GCAGAggtgaagcgaAGTGCAACAC | 5-10-25 | HBV | HBV-related disease | 1.7 | 0.6 | 0.35 | |
| | | AACACAACACAACAC | | | | | | | |
| AUS1221 | 204 | GCAGAggtgaagcgaAGTGCACACA | 5-10-25 | HBV | HBV-related disease | | | | |
| | | CACACACACACACAC | | | | | | | |

Varying Wing (cap) Size with Gap Mod

| | | | | | | | | |
|---|---|---|---|---|---|---|---|---|
| AUS1224 | 205 | GCAGAggtgAagcgaAGUG | 5-4-1-5-4 | HBV | HBV-related disease | 2.7 | 1.2 | 0.46 |
| AUS1225 | 206 | CAGAggtgAagcgaAGUG | 4-4-1-5-4 | HBV | HBV-related disease | 8.8 | 2.2 | 0.26 |
| AUS1226 | 207 | AGAggtgAagcgaAGUG | 3-4-1-5-4 | HBV | HBV-related disease | 19.1 | 3.0 | 0.16 |
| AUS1227 | 208 | CAGAggtgAagcgaAGU | 4-4-1-5-3 | HBV | HBV-related disease | 21.4 | 2.8 | 0.13 |
| AUS1228 | 209 | AGAggtgAagcgaAGU | 3-4-1-5-3 | HBV | HBV-related disease | 32.4 | 4.4 | 0.14 |

Wing Modification

| | | | | | | | | |
|---|---|---|---|---|---|---|---|---|
| AUS1232 | 211 | GCAGAggtgaagcgaAGTGC | 5-10-5 | HBV | HBV-related disease | | | |
| AUS1233 | 10 | GCAGAggtgaagcgaAGTGC | 5-10-5 | HBV | HBV-related disease | | | |
| (reference) | | | | | | | | |
| AUS1234 | 213 | AACAAggtgaagcgaAGTGC | 5-10-5 | HBV | HBV-related disease | 7.0 | 0.7 | 0.10 |
| AUS1235 | 214 | GCAGAggtgaagcgaAACAA | 5-10-5 | HBV | HBV-related disease | 3.4 | 0.6 | 0.17 |
| AUS1236 | 215 | GCAGAggtgaagcgaAGUGC | 5-10-5 | HBV | HBV-related disease | 1.6 | 0.7 | 0.44 |

Wing Modification: 5′ modification Gap+Wing modifications provides optimized and balanced EC50 and CC30

| | | | | | | | | | |
|---|---|---|---|---|---|---|---|---|---|
| AUS1242 | 219 | +GCAGAggtgAagcgaAGTGC | 5-4-1-5-5 | HBV | HBV-related disease | 1.16 | 1.16 | 1.00 | |
| AUS1243 | 220 | +G+CAGAggtgAagcgaAGTGC | 5-4-1-5-5 | HBV | HBV-related disease | 0.79 | 1.14 | 1.44 | |
| AUS1244 | 221 | +GC+AGAggtgAagcgaAGTGC | 5-4-1-5-5 | HBV | HBV-related disease | 0.37 | 1.04 | 2.82 | 77 |
| AUS1245 | 222 | +GCA+GAggtgAagcgaAGTGC | 5-4-1-5-5 | HBV | HBV-related disease | 1.20 | 0.97 | 0.82 | |
| AUS1246 | 223 | +GCAG+AggtgAagcgaAGTGC | 5-4-1-5-5 | HBV | HBV-related disease | 0.84 | 0.92 | 1.10 | |
| AUS1298 | 271 | GcAGAGgtgaAgcgaAGTGC | 6-4-1-4-5 | HBV | HBV-related disease | 1.37 | 1.03 | 0.75 | |
| AUS1299 | 272 | GcAgAGgtgaAgcgaAGTGC | 6-4-1-4-5 | HBV | HBV-related disease | 1.88 | 1.18 | 0.62 | |
| AUS1300 | 273 | GcAGAGgtgaAgcgaAGTGC | 6-4-1-4-5 | HBV | HBV-related disease | 1.04 | 0.89 | 0.85 | |
| AUS1301 | 274 | GcAgAGgtgaAgcgaAGTGC | 6-4-1-4-5 | HBV | HBV-related disease | | | | |
| AUS1302 | 275 | +GCAGAGgtgaAgcgaAGTGC | 6-4-1-4-5 | HBV | HBV-related disease | 1.55 | 0.88 | 0.57 | 73 |
| AUS1303 | 276 | G+CAGAGgtgaAgcgaAGTGC | 6-4-1-4-5 | HBV | HBV-related disease | 0.96 | 1.19 | 1.24 | 74.5 |
| AUS1304 | 277 | GC+AGAGgtgaAgcgaAGTGC | 6-4-1-4-5 | HBV | HBV-related disease | 1.02 | 1.49 | 1.46 | 74.5 |
| AUS1305 | 278 | GCA+GAGgtgaAgcgaAGTGC | 6-4-1-4-5 | HBV | HBV-related disease | 1.33 | 1.78 | 1.34 | 75 |
| AUS1306 | 279 | GCAG+AGgtgaAgcgaAGTGC | 6-4-1-4-5 | HBV | HBV-related disease | 1.46 | 1.12 | 0.77 | 75 |
| AUS1307 | 280 | +GcAGAGgtgaAgcgaAGTGC | 6-4-1-4-5 | HBV | HBV-related disease | 1.78 | 1.01 | 0.57 | 71.5 |
| AUS1308 | 281 | Gc+AGAGgtgaAgcgaAGTGC | 6-4-1-4-5 | HBV | HBV-related disease | 1.23 | 1.02 | 0.83 | 73.5 |
| AUS1309 | 282 | GcA+GAGgtgaAgcgaAGTGC | 6-4-1-4-5 | HBV | HBV-related disease | 1.89 | 1.11 | 0.59 | 74 |
| AUS1310 | 283 | GcAG+AGgtgaAgcgaAGTGC | 6-4-1-4-5 | HBV | HBV-related disease | 1.79 | 0.98 | 0.55 | 73.5 |
| AUS1311 | 284 | +GcAgAGgtgaAgcgaAGTGC | 6-4-1-4-5 | HBV | HBV-related disease | 1.86 | 1.00 | 0.54 | 70 |
| AUS1312 | 285 | Gc+AgAGgtgaAgcgaAGTGC | 6-4-1-4-5 | HBV | HBV-related disease | 1.34 | 1.26 | 0.94 | 72 |
| AUS1313 | 286 | GcAg+AGgtgaAgcgaAGTGC | 6-4-1-4-5 | HBV | HBV-related disease | 1.67 | 1.45 | 0.87 | 72.5 |
| AUS1314 | 287 | +GcAGAggtgAagcgAAGTGC | 5-4-1-4-6 | HBV | HBV-related disease | 2.13 | 1.18 | 0.56 | |
| AUS1315 | 288 | Gc+AGAggtgAagcgAAGTGC | 5-4-1-4-6 | HBV | HBV-related disease | 1.46 | 1.21 | 0.83 | |
| AUS1316 | 289 | GcA+GAggtgAagcgAAGTGC | 5-4-1-4-6 | HBV | HBV-related disease | 1.91 | 1.42 | 0.74 | 72.5 |
| AUS1317 | 290 | GcAG+AggtgAagcgAAGTGC | 5-4-1-4-6 | HBV | HBV-related disease | 1.63 | 1.22 | 0.75 | 71.5 |
| AUS1318 | 291 | +GcAgAggtgAagcgAAGTGC | 5-4-1-4-6 | HBV | HBV-related disease | 1.83 | 1.19 | 0.65 | 68 |
| AUS1319 | 292 | Gc+AgAggtgAagcgAAGTGC | 5-4-1-4-6 | HBV | HBV-related disease | 1.38 | 1.17 | 0.84 | 70 |
| AUS1320 | 293 | GcAg+AggtgAagcgAAGTGC | 5-4-1-4-6 | HBV | HBV-related disease | 2.08 | 1.43 | 0.69 | 70.5 |
| AUS1322 | 294 | +GC+AGAggtgAagcgaAGTGC | 5-4-1-5-5 | HBV | HBV-related disease | 0.76 | 1.15 | 1.52 | |
| AUS1325 | 297 | +GC+AGAggtgAagcgaAGUGC | 5-4-1-5-5 | HBV | HBV-related disease | | | | |
| AUS1326 | 298 | +GC+AGAggtgAagcgaAGUGC | 5-4-1-5-5 | HBV | HBV-related disease | 0.14 | 0.92 | 6.60 | |
| AUS1327 | 299 | G+CA+GAggtgAagcgaAGUGC | 5-4-1-5-5 | HBV | HBV-related disease | 0.11 | 0.98 | 9.20 | |
| AUS1328 | 300 | +G+CA+GAggtgAagcgaAGUGC | 5-4-1-5-5 | HBV | HBV-related disease | 0.10 | 1.00 | 9.79 | |
| AUS1329 | 301 | +GC+AG+AggtgAagcgaAGUGC | 5-4-1-5-5 | HBV | HBV-related disease | 0.09 | 0.88 | 9.97 | |
| AUS1241 | 218 | +G+C+A+G+AggtgaagcgaAGTGC | 5-10-5 | HBV | HBV-related disease | 0.10 | 2.08 | 20.64 | |
| AUS1247 | 224 | G″C″A″G″A″ggtgaagcgaAGTGC | 5-10-5 | HBV | HBV-related disease | 1.18 | 0.39 | 0.33 | |
| AUS1248 | 225 | G″C″A″G″A″ggtgaagcgaAGTGC | 5-10-25 | HBV | HBV-related disease | 1.47 | 0.49 | 0.33 | |
| | | AACACAACACAACACAACAC | | | | | | | |
| AUS1249 | 226 | G″C″G″A″A″gtgcacacggUCCGG | 5-10-5 | HBV | HBV-related disease | 0.96 | 0.75 | 0.78 | |
| AUS1250 | 227 | G″C″A″G″A″ggtgaagcgaAGUGC | 5-10-5 | HBV | HBV-related disease | 1.30 | 0.44 | 0.34 | |
| AUS1251 | 228 | +GC″A″G″A″ggtgaagcgaAGUGC | 5-10-5 | HBV | HBV-related disease | 1.23 | 0.53 | 0.43 | |
| AUS1252 | 229 | +G+CA″G″A″ggtgaagcgaAGUGC | 5-10-5 | HBV | HBV-related disease | 0.72 | 0.71 | 0.99 | |
| AUS1253 | 230 | +GC″+AG″A″ggtgaagcgaAGUGC | 5-10-5 | HBV | HBV-related disease | 0.45 | 0.59 | 1.33 | |
| AUS1254 | 231 | +GC″A″+GA″ggtgaagcgaAGUGC | 5-10-5 | HBV | HBV-related disease | 0.39 | 0.42 | 1.09 | |
| AUS1255 | 232 | +GC″A″G″+AggtgaagcgaAGUGC | 5-10-5 | HBV | HBV-related disease | 0.50 | 0.41 | 0.83 | |
| AUS1256 | 233 | +G+C+AG″A″ggtgaagcgaAGUGC | 5-10-5 | HBV | HBV-related disease | 0.52 | 0.64 | 1.25 | |
| AUS1257 | 234 | +GC″AG″AggtgaagcgaAGUGC | 5-10-5 | HBV | HBV-related disease | 0.97 | 0.93 | 0.96 | |
| AUS1258 | 235 | +GCA″GA″ggtgaagcgaAGUGC | 5-10-5 | HBV | HBV-related disease | 1.26 | 0.87 | 0.69 | |
| AUS1259 | 236 | +G+CA″GA″ggtgaagcgaAGUGC | 5-10-5 | HBV | HBV-related disease | 0.76 | 0.73 | 0.95 | |
| AUS1260 | 237 | +G+CAG″AggtgaagcgaAGUGC | 5-10-5 | HBV | HBV-related disease | 0.61 | 0.82 | 1.34 | |
| AUS1261 | 238 | +GCAGAggtgaagcgaAGUGC | 5-10-5 | HBV | HBV-related disease | 1.10 | 1.11 | 1.02 | |
| AUS1262 | 239 | +G+CAGAggtgaagcgaAGUGC | 5-10-5 | HBV | HBV-related disease | 0.94 | 1.16 | 1.24 | |
| AUS1263 | 240 | +G+C+AGAggtgaagcgaAGUGC | 5-10-5 | HBV | HBV-related disease | 0.50 | 1.04 | 2.09 | |
| AUS1264 | 241 | +G+C+A+GAggtgaagcgaAGUGC | 5-10-5 | HBV | HBV-related disease | 0.45 | 0.96 | 2.12 | |
| AUS1266 | 242 | GGAGAggtgaagcgaAGTGC | 5-10-5 | HBV | HBV-related disease | 2.04 | 3.37 | 1.65 | |
| AUS1267 | 243 | GCTGAggtgaagcgaAGTGC | 5-10-5 | HBV | HBV-related disease | 1.25 | 0.98 | 0.78 | |
| AUS1268 | 244 | GCACAggtgaagcgaAGTGC | 5-10-5 | HBV | HBV-related disease | 1.81 | 1.18 | 0.65 | |
| AUS1269 | 245 | GCAGTggtgaagcgaAGTGC | 5-10-5 | HBV | HBV-related disease | 1.06 | 1.06 | 1.00 | |
| AUS1270 | 246 | GCAGAcgtgaagcgaAGTGC | 5-10-5 | HBV | HBV-related disease | 1.52 | 1.31 | 0.86 | |
| AUS1271 | 247 | GCAGAgctgaagcgaAGTGC | 5-10-5 | HBV | HBV-related disease | 2.03 | 1.38 | 0.68 | |
| AUS1272 | 248 | CCAGAggtgaagcgaAGTGC | 5-10-5 | HBV | HBV-related disease | 2.78 | 1.34 | 0.48 | |
| AUS1274 | 249 | G″C″A″G″A″ggtgaagcgaAGUGC | 5-10-5 | HBV | HBV-related disease | 1.32 | 0.45 | 0.34 | |
| AUS1275 | 250 | GCA″G″A″ggtgaagcgaAGUGC | 5-10-5 | HBV | HBV-related disease | 0.97 | 0.34 | 0.35 | |
| AUS1277 | 251 | +G+CAGAggtgaagcgaAGTGC | 5-10-5 | HBV | HBV-related disease | 0.48 | 0.81 | 1.69 | |
| AUS1278 | 252 | +GC+AGAggtgaagcgaAGTGC | 5-10-5 | HBV | HBV-related disease | 0.29 | 1.02 | 3.48 | |
| AUS1279 | 253 | +GCA+GAggtgaagcgaAGTGC | 5-10-5 | HBV | HBV-related disease | 0.55 | 0.72 | 1.31 | |
| AUS1280 | 254 | +GCAG+AggtgaagcgaAGTGC | 5-10-5 | HBV | HBV-related disease | 0.51 | 0.67 | 1.31 | |
| AUS1281 | 255 | G+C+AGAggtgaagcgaAGTGC | 5-10-5 | HBV | HBV-related disease | 0.40 | 0.95 | 2.39 | |
| AUS1282 | 256 | G+CA+GAggtgaagcgaAGTGC | 5-10-5 | HBV | HBV-related disease | 0.29 | 0.85 | 2.92 | |
| AUS1283 | 257 | G+CAG+AggtgaagcgaAGTGC | 5-10-5 | HBV | HBV-related disease | 0.21 | 0.58 | 2.73 | |
| AUS1284 | 258 | GC+A+GAggtgaagcgaAGTGC | 5-10-5 | HBV | HBV-related disease | 0.28 | 0.92 | 3.22 | |
| AUS1285 | 259 | GC+AG+AggtgaagcgaAGTGC | 5-10-5 | HBV | HBV-related disease | 0.18 | 0.61 | 3.35 | |
| AUS1286 | 260 | GCA+G+AggtgaagcgaAGTGC | 5-10-5 | HBV | HBV-related disease | 0.38 | 0.66 | 1.75 | |
| AUS1287 | 261 | +G+C+AGAggtgaagcgaAGTGC | 5-10-5 | HBV | HBV-related disease | 0.38 | 0.94 | 2.44 | |
| AUS1288 | 262 | +G+CA+GAggtgaagcgaAGTGC | 5-10-5 | HBV | HBV-related disease | 0.21 | 0.98 | 4.65 | |
| AUS1289 | 263 | +G+CAG+AggtgaagcgaAGTGC | 5-10-5 | HBV | HBV-related disease | 0.30 | 0.62 | 2.06 | |
| AUS1290 | 264 | +GC+A+GAggtgaagcgaAGTGC | 5-10-5 | HBV | HBV-related disease | 0.25 | 0.84 | 3.41 | |
| AUS1291 | 265 | +GC+AG+AggtgaagcgaAGTGC | 5-10-5 | HBV | HBV-related disease | 0.11 | 0.56 | 5.05 | |
| AUS1292 | 266 | +GCA+G+AggtgaagcgaAGTGC | 5-10-5 | HBV | HBV-related disease | 0.15 | 0.58 | 3.88 | |
| AUS1293 | 267 | G+C+A+GAggtgaagcgaAGTGC | 5-10-5 | HBV | HBV-related disease | 0.18 | 0.65 | 3.63 | |
| AUS1294 | 268 | G+C+AG+AggtgaagcgaAGTGC | 5-10-5 | HBV | HBV-related disease | 0.12 | 0.47 | 3.74 | |
| AUS1295 | 269 | G+CA+G+AggtgaagcgaAGTGC | 5-10-5 | HBV | HBV-related disease | 0.14 | 0.44 | 3.04 | |
| AUS1296 | 270 | GC+A+G+AggtgaagcgaAGTGC | 5-10-5 | HBV | HBV-related disease | 0.17 | 0.58 | 3.44 | |
| AUS1323 | 295 | +G+CAGAggtgaagcgaAGTGC | 5-10-5 | HBV | HBV-related disease | 0.94 | 1.32 | 1.41 | |
| AUS1324 | 296 | +GC+AGAggtgaagcgaAGTGC | 5-10-5 | HBV | HBV-related disease | 1.12 | 1.30 | 1.16 | |

Wing Modification: 3′ modification

| | | | | | | | | |
|---|---|---|---|---|---|---|---|---|
| AUS1340 | 309 | GCAGAGgtgaAgcga+A+G+TGC | 6-4-1-4-5 | HBV | HBV-related disease | 0.29 | 0.80 | 2.80 |
| AUS1341 | 310 | GCAGAGgtgaAgcga+A+GT+GC | 6-4-1-4-5 | HBV | HBV-related disease | 0.26 | 0.70 | 2.64 |
| AUS1342 | 311 | GCAGAGgtgaAgcga+A+GTG+C | 6-4-1-4-5 | HBV | HBV-related disease | 0.40 | 0.63 | 1.58 |
| AUS1343 | 312 | GCAGAGgtgaAgcga+AG+T+GC | 6-4-1-4-5 | HBV | HBV-related disease | 0.37 | 0.85 | 2.31 |
| AUS1344 | 313 | GCAGAGgtgaAgcga+AG+TG+C | 6-4-1-4-5 | HBV | HBV-related disease | 0.29 | 0.83 | 2.84 |
| AUS1345 | 314 | GCAGAGgtgaAgcga+AGT+G+C | 6-4-1-4-5 | HBV | HBV-related disease | 0.24 | 0.65 | 2.63 |
| AUS1346 | 315 | GCAGAGgtgaAgcgaA+G+T+GC | 6-4-1-4-5 | HBV | HBV-related disease | 0.15 | 0.59 | 3.94 |
| AUS1347 | 316 | GCAGAGgtgaAgcgaA+G+TG+C | 6-4-1-4-5 | HBV | HBV-related disease | 0.22 | 0.44 | 2.01 |
| AUS1348 | 317 | GCAGAGgtgaAgcgaA+GT+G+C | 6-4-1-4-5 | HBV | HBV-related disease | 0.22 | 0.84 | 3.80 |
| AUS1349 | 318 | GCAGAGgtgaAgcgaAG+T+G+C | 6-4-1-4-5 | HBV | HBV-related disease | 0.14 | 0.59 | 4.34 |
| AUS1350 | 319 | GCAGAggtgAagcgA+A+G+TGC | 5-4-1-4-6 | HBV | HBV-related disease | 0.15 | 0.57 | 3.80 |
| AUS1351 | 320 | GCAGAggtgAagcgA+A+GT+GC | 5-4-1-4-6 | HBV | HBV-related disease | 0.23 | 0.66 | 2.89 |
| AUS1352 | 321 | GCAGAggtgAagcgA+A+GTG+C | 5-4-1-4-6 | HBV | HBV-related disease | 0.16 | 0.73 | 4.59 |
| AUS1353 | 322 | GCAGAggtgAagcgA+AG+T+GC | 5-4-1-4-6 | HBV | HBV-related disease | 0.15 | 0.51 | 3.48 |
| AUS1354 | 323 | GCAGAggtgAagcgA+AG+TG+C | 5-4-1-4-6 | HBV | HBV-related disease | 0.12 | 0.40 | 3.44 |
| AUS1355 | 324 | GCAGAggtgAagcgA+AGT+G+C | 5-4-1-4-6 | HBV | HBV-related disease | 0.13 | 0.60 | 4.61 |
| AUS1356 | 325 | GCAGAggtgAagcgAA+G+T+GC | 5-4-1-4-6 | HBV | HBV-related disease | 0.19 | 0.73 | 3.80 |
| AUS1357 | 326 | GCAGAggtgAagcgAA+G+TG+C | 5-4-1-4-6 | HBV | HBV-related disease | 0.14 | 0.56 | 4.02 |
| AUS1358 | 327 | GCAGAggtgAagcgAA+GT+G+C | 5-4-1-4-6 | HBV | HBV-related disease | 0.12 | 0.60 | 4.86 |
| AUS1359 | 328 | GCAGAggtgAagcgAAG+T+G+C | 5-4-1-4-6 | HBV | HBV-related disease | 0.32 | 0.71 | 2.23 |
| AUS1360 | 329 | GCAGAggtgAagcga+A+GUGC | 5-4-1-5-5 | HBV | HBV-related disease | 0.09 | 0.80 | 8.61 |
| AUS1361 | 330 | GCAGAggtgAagcgaA+GU+G+C | 5-4-1-5-5 | HBV | HBV-related disease | 0.13 | 0.89 | 6.85 |
| AUS1362 | 331 | GCAGAggtgAagcgaA+G+T+GC | 5-4-1-5-5 | HBV | HBV-related disease | 0.10 | 0.99 | 9.71 |
| AUS1332 | 302 | GCAGAggtgaagcga+A+G+T+G+C | 5-10-5 | HBV | HBV-related disease | 0.12 | 3.04 | 25.45 |
| AUS1333 | 303 | GCGAAgtgcacacggU″C″C″G″G″ | 5-10-5 | HBV | HBV-related disease | 0.6 | 0.6 | 0.90 |
| AUS1334 | 304 | GCAGAggtgaagcgaA″G″U″G″C″ | 5-10-5 | HBV | HBV-related disease | 0.7 | 0.6 | 0.90 |
| AUS1335 | 305 | GCAGAggtgaagcga+AGUGC | 5-10-5 | HBV | HBV-related disease | 0.8 | 1.3 | 1.53 |
| AUS1336 | 306 | GCAGAggtgaagcga+A+GUGC | 5-10-5 | HBV | HBV-related disease | 0.8 | 1.7 | 2.05 |
| AUS1337 | 307 | GCAGAggtgaagcga+A+G+TGC | 5-10-5 | HBV | HBV-related disease | 0.4 | 1.3 | 3.04 |
| AUS1338 | 308 | GCAGAggtgaagcga+A+G+T+GC | 5-10-5 | HBV | HBV-related disease | 0.3 | 0.8 | 2.59 |

Wing Modification: 5′ + 3′ modification

| | | | | | | | | |
|---|---|---|---|---|---|---|---|---|
| AUS1370 | 336 | +Gc+AGAGgtgaAgcga+A+GUGC | 6-4-1-4-5 | HBV | HBV-related disease | 0.74 | 1.10 | 1.49 |
| AUS1371 | 337 | +Gc+AgAGgtgaAgcga+A+GUGC | 6-4-1-4-5 | HBV | HBV-related disease | 1.66 | 1.47 | 0.89 |
| AUS1372 | 338 | +Gc+AGAggtgAagcgA+A+GUGC | 5-4-1-4-6 | HBV | HBV-related disease | 0.92 | 1.44 | 1.56 |
| AUS1373 | 339 | +Gc+AgAggtgAagcgA+A+GUGC | 5-4-1-4-6 | HBV | HBV-related disease | 0.24 | 1.34 | 5.62 |
| AUS1374 | 340 | Gc+AGAGgtgaAgcga+A+G+TGC | 6-4-1-4-5 | HBV | HBV-related disease | 0.74 | 1.12 | 1.51 |
| AUS1375 | 341 | Gc+AGAGgtgaAgcga+AG+TG+C | 6-4-1-4-5 | HBV | HBV-related disease | 1.02 | 0.89 | 0.87 |
| AUS1376 | 342 | GC+AGAGgtgaAgcga+AG+TG+C | 6-4-1-4-5 | HBV | HBV-related disease | 0.56 | 0.93 | 1.66 |
| AUS1377 | 343 | Gc+AgAGgtgaAgcga+AG+TG+C | 6-4-1-4-5 | HBV | HBV-related disease | 0.27 | 1.06 | 3.93 |
| AUS1378 | 344 | Gc+AGAggtgAagcgA+A+G+TGC | 5-4-1-4-6 | HBV | HBV-related disease | 0.48 | 0.93 | 1.93 |
| AUS1379 | 345 | Gc+AGAggtgAagcgA+AG+TG+C | 5-4-1-4-6 | HBV | HBV-related disease | 0.77 | 0.69 | 0.89 |
| AUS1365 | 332 | +G+C+A+G+Aggtgaagcga+A+G+ | 5-10-5 | HBV | HBV-related disease | 0.08 | 1.94 | 24.34 |
| | | T+G+C | | | | | | |
| AUS1367 | 334 | +GC+AGAggtgaagcgaA ″G″U″G″ | 5-10-5 | HBV | HBV-related disease | 0.23 | 0.63 | 2.72 |
| | | C″ | | | | | | |
| AUS1368 | 335 | +GC+A+GAggtgaagcgaA″G″U″G | 5-10-5 | HBV | HBV-related disease | | | |
| | | ″C″ | | | | | | |
| AUS1366 | 333 | +G+CAGAGGTGAAGCGAAGT+G+C | n/a | HBV | HBV-related disease | 24.42 | >250 | |

Different Targets within HBV

| | | | | | | | | |
|---|---|---|---|---|---|---|---|---|
| AUS1697 | 653 | GTGAAgcGaagtgcaCACGG | 5-2-1-7-5 | HBV | HBV-related disease | | | |
| AUS1698 | 654 | GTGAAgcgaAgtgcaCACGG | 5-4-1-5-5 | HBV | HBV-related disease | | | |
| AUS1699 | 655 | GTGAAgcgaaGtgcaCACGG | 5-5-1-4-5 | HBV | HBV-related disease | | | |
| AUS1700 | 656 | GTGAAgcGaaGtgcaCACGG | 5-2-1-2-1- | HBV | HBV-related disease | | | |
| | | | 4-5 | | | | | |
| AUS1505 | 466 | AGAGGcaggagtgccTAAGG | 5-10-5 | HBV | HBV-related disease | 29.7 | 0.6 | 0.02 |
| AUS1506 | 467 | CCGUGtgcacttcgcUUCAC | 5-10-5 | HBV | HBV-related disease | | | |
| AUS1507 | 468 | CUGGCtcagtttactAGUGC | 5-10-5 | HBV | HBV-related disease | | | |
| AUS1508 | 469 | GCCGAtccatactgGGAAC | 5-9-5 | HBV | HBV-related disease | | | |
| AUS1509 | 470 | GUGGActtctctcaaUUUUC | 5-10-5 | HBV | HBV-related disease | | | |
| AUS1510 | 471 | CGCUGtaggcataaaUUGGU | 5-10-5 | HBV | HBV-related disease | | | |
| AUS1511 | 472 | UGACAagaatcctcaCAAUA | 5-10-5 | HBV | HBV-related disease | | | |
| AUS1512 | 473 | GACAAgaatcctcacAAUAC | 5-10-5 | HBV | HBV-related disease | | | |
| AUS1513 | 474 | AGCgaagtgcacaCGG | 3-10-3 | HBV | HBV-related disease | | | |
| AUS1514 | 475 | UUCUUcttctaggggACCUG | 5-10-5 | HBV | HBV-related disease | | | |
| AUS1515 | 476 | ACAAAcgggcaacatACCUU | 5-10-5 | HBV | HBV-related disease | | | |
| AUS1516 | 477 | GGAUUcagcgccgacGGGAC | 5-10-5 | HBV | HBV-related disease | | | |
| AUS1517 | 478 | CGUGGtggacttctCUCAA | 5-9-5 | HBV | HBV-related disease | | | |
| AUS1518 | 479 | CCUUCcctgaaggttCCUCC | 5-10-5 | HBV | HBV-related disease | | | |
| AUS1519 | 480 | CACTAgtaaactgaGCCAG | 5-9-5 | HBV | HBV-related disease | | | |
| AUS1520 | 481 | TTCCGcagtatggaTCGGC | 5-9-5 | HBV | HBV-related disease | | | |
| AUS1521 | 482 | AGAAAattgagagaagTCCAC | 5-11-5 | HBV | HBV-related disease | | | |
| AUS1522 | 483 | ACCAAtttatgcctACAGC | 5-9-5 | HBV | HBV-related disease | | | |
| AUS1523 | 484 | TATTGtgaggattctTGTCA | 5-10-5 | HBV | HBV-related disease | | | |
| AUS1524 | 485 | GTATTgtgaggattcTTGTC | 5-10-5 | HBV | HBV-related disease | | | |
| AUS1525 | 486 | CCTTCcctgaaggttCCTCC | 5-10-5 | HBV | HBV-related disease | | | |
| AUS1526 | 487 | TTCTTcttctaggggACCTG | 5-10-5 | HBV | HBV-related disease | | | |
| AUS1527 | 488 | ACAAAcgggcaacatACCTT | 5-10-5 | HBV | HBV-related disease | | | |
| AUS1528 | 489 | GGATTcagcgccgacGGGAC | 5-10-5 | HBV | HBV-related disease | | | |
| AUS1529 | 490 | ATTGAgagaagtccaCCACG | 5-10-5 | HBV | HBV-related disease | | | |
| AUS1530 | 491 | AGAAAattgagagaaGTCCA | 5-10-5 | HBV | HBV-related disease | >250 | >250 | |
| AUS1531 | 492 | GAAAAttgagagaagTCCAC | 5-10-5 | HBV | HBV-related disease | >250 | >250 | |
| AUS1532 | 493 | AAAATtgagagaagtCCACC | 5-10-5 | HBV | HBV-related disease | >250 | >250 | |
| AUS1533 | 494 | AAATTgagagaagtcCACCA | 5-10-5 | HBV | HBV-related disease | >250 | >250 | |
| AUS1534 | 495 | AATTGagagaagtccACCAC | 5-10-5 | HBV | HBV-related disease | >250 | >250 | |
| AUS1535 | 496 | ATTGAgagaagtccaCCACG | 5-10-5 | HBV | HBV-related disease | 35.9 | >250 | |
| AUS1536 | 497 | CAGAGgtgaagcgaaGTGCA | 5-10-5 | HBV | HBV-related disease | 1.71 | >250 | |
| AUS1537 | 498 | AGAGGtgaagcgaagTGCAC | 5-10-5 | HBV | HBV-related disease | 1.89 | >250 | |
| AUS1538 | 499 | GAGGTgaagcgaagtGCACA | 5-10-5 | HBV | HBV-related disease | 3.33 | >250 | |
| AUS1539 | 500 | AGGTGaagcgaagtgCACAC | 5-10-5 | HBV | HBV-related disease | 5.75 | >250 | |
| AUS1540 | 501 | GGTGAagcgaagtgcACACG | 5-10-5 | HBV | HBV-related disease | 3.29 | >250 | |
| AUS1541 | 502 | GTGAAgcgaagtgcaCACGG | 5-10-5 | HBV | HBV-related disease | 1.69 | >250 | |
| AUS1542 | 503 | GCACTagtaaactgaGCCAG | 5-10-5 | HBV | HBV-related disease | 1.04 | >250 | |
| AUS1543 | 504 | GTTCCgcagtatggaTCGGC | 5-10-5 | HBV | HBV-related disease | 3.98 | >250 | |
| AUS1544 | 505 | ACCAAtttatgcctaCAGCC | 5-10-5 | HBV | HBV-related disease | >250 | >250 | |
| AUS1545 | 506 | TATTGtgaggattctTGTCA | 5-10-5 | HBV | HBV-related disease | 35.96 | >250 | |
| AUS1546 | 507 | TGCGGtattgtgaggattcttgtca | 5-20-5 | HBV | HBV-related disease | 1.65 | 0.76 | 0.46 |
| | | ACAAG | | | | | | |
| AUS1547 | 508 | AGCgaagtgcacaCGG | 3-10-3 | HBV | HBV-related disease | >250 | >250 | |
| AUS1548 | 509 | CCTTCcctgaaggttCCTCC | 5-10-5 | HBV | HBV-related disease | >250 | >250 | |
| AUS1549 | 510 | TTCTTcttctaggggACCTG | 5-10-5 | HBV | HBV-related disease | >250 | >250 | |
| AUS1550 | 511 | ACAAAcgggcaacatACCTT | 5-10-5 | HBV | HBV-related disease | >250 | >250 | |
| AUS1551 | 512 | GTGAAgcgaagtgcaCCAAC | 5-10-5 | HBV | HBV-related disease | 9.57 | >250 | |
| AUS1552 | 513 | CAACAgcgaagtgcaCACGG | 5-10-5 | HBV | HBV-related disease | 6.70 | >250 | |
| AUS1553 | 514 | CTGCGgtattgtgagGATTC | 5-10-5 | HBV | HBV-related disease | | | |
| AUS1554 | 515 | GCGGTattgtgaggaTTCTT | 5-10-5 | HBV | HBV-related disease | | | |
| AUS1555 | 516 | GGTATtgtgaggattCTTGT | 5-10-5 | HBV | HBV-related disease | | | |
| AUS1556 | 517 | TATTGtgaggattctTGTCA | 5-10-5 | HBV | HBV-related disease | | | |
| AUS1557 | 518 | TTGTGaggattcttgTCAAC | 5-10-5 | HBV | HBV-related disease | | | |
| AUS1558 | 519 | GTGAGgattcttgtcAACAA | 5-10-5 | HBV | HBV-related disease | | | |
| AUS1559 | 520 | GAGGAttcttgtcaaCAAGA | 5-10-5 | HBV | HBV-related disease | | | |
| AUS1560 | 521 | AAATGgcactagtaaACTGA | 5-10-5 | HBV | HBV-related disease | | | |
| AUS1561 | 522 | ATGGCactagtaaacTGAGC | 5-10-5 | HBV | HBV-related disease | | | |
| AUS1562 | 523 | GGCACtagtaaactgAGCCA | 5-10-5 | HBV | HBV-related disease | | | |
| AUS1563 | 524 | CACTAgtaaactgagCCAGG | 5-10-5 | HBV | HBV-related disease | | | |
| AUS1564 | 525 | CTAGTaaactgagccAGGAG | 5-10-5 | HBV | HBV-related disease | | | |
| AUS1565 | 526 | AGTAAactgagccagGAGAA | 5-10-5 | HBV | HBV-related disease | | | |
| AUS1566 | 527 | TAGGAgttccgcagtATGGA | 5-10-5 | HBV | HBV-related disease | | | |
| AUS1567 | 528 | GGAGTtccgcagtatGGATC | 5-10-5 | HBV | HBV-related disease | | | |
| AUS1568 | 529 | AGTTCcgcagtatggATCGG | 5-10-5 | HBV | HBV-related disease | | | |
| AUS1569 | 530 | TTCCGcagtatggatCGGCA | 5-10-5 | HBV | HBV-related disease | | | |
| AUS1570 | 531 | CCGCAgtatggatcgGCAGA | 5-10-5 | HBV | HBV-related disease | | | |
| AUS1571 | 532 | GCAGTatggatcggcAGAGG | 5-10-5 | HBV | HBV-related disease | | | |
| AUS1572 | 533 | AAAUGgcactagtaaactgagccag | 5-20-5 | HBV | HBV-related disease | | | |
| | | GAGAA | | | | | | |
| AUS1573 | 534 | UAGGAgttccgcagtatggatcggc | 5-20-5 | HBV | HBV-related disease | | | |
| | | AGAGG | | | | | | |
| AUS1574 | 535 | CGCAGaccaatttatgcctacagcc | 5-20-5 | HBV | HBV-related disease | | | |
| | | UCCUA | | | | | | |
| AUS1575 | 536 | AGAGGacaaacgggcaacatacctt | 5-20-5 | HBV | HBV-related disease | | | |
| | | GAUAG | | | | | | |
| AUS1576 | 537 | GGAUUcagcgccgacGGGAC | 5-10-5 | HBV | HBV-related disease | | | |
| AUS1577 | 538 | CCGCAggattcagcgccgacgggac | 5-20-5 | HBV | HBV-related disease | | | |
| | | GUAAA | | | | | | |
| AUS1578 | 539 | CCCCTagaaaattgagagaaGTCCA | 5-15-5 | HBV | HBV-related disease | 2.9 | 1.8 | 0.62 |
| AUS1579 | 540 | CCCCTagaaaattgagagaagtcca | 5-20-5 | HBV | HBV-related disease | 2.0 | 0.5 | 0.24 |
| | | CCACG | | | | | | |
| AUS1580 | 541 | AGAAAattgagagaagtccaCCACG | 5-15-5 | HBV | HBV-related disease | 6.9 | 1.1 | 0.16 |
| AUS1581 | 542 | AGAAAattgagagaagtccaccacg | 5-20-5 | HBV | HBV-related disease | 3.2 | 0.4 | 0.11 |
| | | AGTCT | | | | | | |
| AUS1582 | 543 | TGAAGcgaagtgcacACGGT | 5-10-5 | HBV | HBV-related disease | 2.00 | 2.82 | 1.41 |
| AUS1583 | 544 | GAAGCgaagtgcacaCGGTC | 5-10-5 | HBV | HBV-related disease | 2.16 | 0.82 | 0.38 |
| AUS1584 | 545 | AAGCGaagtgcacacGGTCC | 5-10-5 | HBV | HBV-related disease | 1.08 | 0.80 | 0.74 |
| AUS1585 | 546 | AGCGAagtgcacacgGTCCG | 5-10-5 | HBV | HBV-related disease | 1.19 | 0.82 | 0.69 |
| AUS1586 | 547 | GCGAAgtgcacacggTCCGG | 5-10-5 | HBV | HBV-related disease | 0.49 | 0.91 | 1.88 |
| AUS1587 | 548 | ATGGCACTagtaaactgaGCCAGGA | 8-10-8 | HBV | HBV-related disease | 0.94 | 1.78 | 1.90 |
| | | G | | | | | | |
| AUS1588 | 549 | TGGCACTagtaaactgaGCCAGGA | 7-10-7 | HBV | HBV-related disease | 4.31 | 1.07 | 0.25 |
| AUS1589 | 550 | GGCACTagtaaactgaGCCAGG | 6-10-6 | HBV | HBV-related disease | 0.99 | 1.41 | 1.42 |
| AUS1590 | 551 | CACTagtaaactgaGCCA | 4-10-4 | HBV | HBV-related disease | 2.86 | 3.82 | 1.33 |
| AUS1592 | 553 | AGACTctgcggtattGTGAG | 5-10-5 | HBV | HBV-related disease | 5.7 | 3.1 | 0.53 |
| AUS1593 | 554 | GACTCtgcggtattgTGAGG | 5-10-5 | HBV | HBV-related disease | 4.0 | 2.6 | 0.67 |
| AUS1594 | 555 | ACTCTgcggtattgtGAGGA | 5-10-5 | HBV | HBV-related disease | 2.6 | 1.8 | 0.68 |
| AUS1595 | 556 | CTCTGcggtattgtgAGGAT | 5-10-5 | HBV | HBV-related disease | 2.7 | 1.2 | 0.45 |
| AUS1596 | 557 | TCTGCggtattgtgaGGATT | 5-10-5 | HBV | HBV-related disease | 2.8 | 3.5 | 1.25 |
| AUS1597 | 558 | CTGCGgtattgtgagGATTC | 5-10-5 | HBV | HBV-related disease | 1.3 | 1.6 | 1.21 |
| AUS1598 | 559 | TGCGGtattgtgaggATTCT | 5-10-5 | HBV | HBV-related disease | 1.9 | 1.5 | 0.77 |
| AUS1599 | 560 | GCGGTattgtgaggaTTCTT | 5-10-5 | HBV | HBV-related disease | 1.6 | 0.4 | 0.27 |
| AUS1600 | 561 | CGGTAttgtgaggatTCTTG | 5-10-5 | HBV | HBV-related disease | 1.8 | 0.8 | 0.46 |
| AUS1601 | 562 | GGCACtagtaaactgAGCCA | 5-10-5 | HBV | HBV-related disease | 7.5 | 8.9 | 1.18 |
| AUS1602 | 563 | GCACTagtaaactgaGCCAG | 5-10-5 | HBV | HBV-related disease | 1.2 | 0.6 | 0.55 |
| AUS1603 | 564 | CACTAgtaaactgagCCAGG | 5-10-5 | HBV | HBV-related disease | 0.9 | 0.9 | 0.99 |
| AUS1604 | 565 | ACTAGtaaactgagcCAGGA | 5-10-5 | HBV | HBV-related disease | 1.3 | 1.1 | 0.85 |
| AUS1605 | 566 | TTCCGcagtatggatCGGCA | 5-10-5 | HBV | HBV-related disease | 1.7 | 7.4 | 4.40 |
| AUS1606 | 567 | TCCGCagtatggatcGGCAG | 5-10-5 | HBV | HBV-related disease | 1.1 | 1.2 | 1.04 |
| AUS1607 | 568 | CCGCAgtatggatcgGCAGA | 5-10-5 | HBV | HBV-related disease | 1.8 | 1.8 | 1.00 |
| AUS1608 | 569 | CGCAGtatggatcggCAGAG | 5-10-5 | HBV | HBV-related disease | 2.1 | 3.0 | 1.44 |
| AUS1609 | 570 | CGAAGtgcacacggtCCGGC | 5-10-5 | HBV | HBV-related disease | 1.0 | 0.8 | 0.78 |
| AUS1610 | 571 | GAAGTgcacacggtcCGGCA | 5-10-5 | HBV | HBV-related disease | 1.5 | 1.1 | 0.75 |
| AUS1611 | 572 | AAGTGcacacggtccGGCAG | 5-10-5 | HBV | HBV-related disease | 1.4 | 1.2 | 0.86 |
| AUS1612 | 573 | GTGCAcacggtccggCAGAT | 5-10-5 | HBV | HBV-related disease | 1.0 | 0.5 | 0.51 |
| AUS1613 | 574 | GCACAcggtccggcaGATGA | 5-10-5 | HBV | HBV-related disease | 1.2 | 0.7 | 0.56 |
| AUS1614 | 575 | ACACGgtccggcagaTGAGA | 5-10-5 | HBV | HBV-related disease | 2.9 | 1.3 | 0.46 |
| AUS1615 | 576 | ACGGTccggcagatgAGAAG | 5-10-5 | HBV | HBV-related disease | 2.4 | 2.3 | 0.96 |
| AUS1616 | 577 | GGTCCggcagatgagAAGGC | 5-10-5 | HBV | HBV-related disease | 1.4 | 2.6 | 1.88 |
| AUS1617 | 578 | TCCGGcagatgagaaGGCAC | 5-10-5 | HBV | HBV-related disease | 1.3 | 1.8 | 1.32 |
| AUS1618 | 579 | CGGCAgatgagaaggCACAG | 5-10-5 | HBV | HBV-related disease | 2.3 | 0.8 | 0.37 |
| AUS1619 | 580 | GCAGAtgagaaggcaCAGAC | 5-10-5 | HBV | HBV-related disease | 5.9 | 4.3 | 0.72 |
| AUS1620 | 581 | AGATGagaaggcacaGACGG | 5-10-5 | HBV | HBV-related disease | 6.7 | >250 | |
| AUS1621 | 582 | ATGAGaaggcacagaCGGGG | 5-10-5 | HBV | HBV-related disease | 11.7 | 0.8 | 0.07 |
| AUS1622 | 583 | GAGAAggcacagacgGGGAG | 5-10-5 | HBV | HBV-related disease | 17.6 | 4.1 | 0.23 |
| AUS1623 | 584 | GAAGGcacagacgggGAGTC | 5-10-5 | HBV | HBV-related disease | 11.9 | >250 | |
| AUS1624 | 585 | AGGCAcagacggggaGTCCG | 5-10-5 | HBV | HBV-related disease | 1.5 | >250 | |
| AUS1625 | 586 | GCACAgacggggagtCCGCG | 5-10-5 | HBV | HBV-related disease | 1.1 | >250 | |
| AUS1626 | 587 | ACAGAcggggagtccGCGTA | 5-10-5 | HBV | HBV-related disease | 4.2 | >250 | |
| AUS1627 | 588 | AGACGgggagtccgcGTAAA | 5-10-5 | HBV | HBV-related disease | 7.1 | 2.4 | 0.33 |
| AUS1628 | 589 | ACGGGgagtccgcgtAAAGA | 5-10-5 | HBV | HBV-related disease | 11.4 | 2.7 | 0.24 |
| AUS1629 | 590 | GGGGAgtccgcgtaaAGAGA | 5-10-5 | HBV | HBV-related disease | 20.8 | 3.9 | 0.19 |
| AUS1630 | 591 | GGAGTccgcgtaaagAGAGG | 5-10-5 | HBV | HBV-related disease | 2.4 | >250 | |
| AUS1631 | 592 | GGTCGtccgcaggatTCAGC | 5-10-5 | HBV | HBV-related disease | | | |
| AUS1632 | 593 | GTCGTccgcaggattCAGCG | 5-10-5 | HBV | HBV-related disease | | | |
| AUS1633 | 594 | TCGTCcgcaggattcAGCGC | 5-10-5 | HBV | HBV-related disease | | | |
| AUS1634 | 595 | CGTCCgcaggattcaGCGCC | 5-10-5 | HBV | HBV-related disease | | | |
| AUS1635 | 596 | GTCCGcaggattcagCGCCG | 5-10-5 | HBV | HBV-related disease | | | |
| AUS1636 | 597 | TCCGCaggattcagcGCCGA | 5-10-5 | HBV | HBV-related disease | | | |
| AUS1637 | 598 | CCGCAggattcagcgCCGAC | 5-10-5 | HBV | HBV-related disease | | | |
| AUS1638 | 599 | CGCAGgattcagcgcCGACG | 5-10-5 | HBV | HBV-related disease | | | |
| AUS1639 | 600 | GCAGGattcagcgccGACGG | 5-10-5 | HBV | HBV-related disease | | | |
| AUS1640 | 601 | CAGGAttcagcgccgACGGG | 5-10-5 | HBV | HBV-related disease | | | |
| AUS1641 | 602 | AGGATtcagcgccgaCGGGA | 5-10-5 | HBV | HBV-related disease | | | |
| AUS1642 | 603 | GATTCagcgccgacgGGACG | 5-10-5 | HBV | HBV-related disease | | | |
| AUS1643 | 604 | ATTCAgcgccgacggGACGT | 5-10-5 | HBV | HBV-related disease | | | |
| AUS1644 | 605 | TTCAGcgccgacgggACGTA | 5-10-5 | HBV | HBV-related disease | | | |
| AUS1645 | 606 | TCAGCgccgacgggaCGTAA | 5-10-5 | HBV | HBV-related disease | | | |
| AUS1646 | 607 | CAGCGccgacgggacGTAAA | 5-10-5 | HBV | HBV-related disease | | | |
| AUS1647 | 608 | AGCGCcgacgggacgTAAAC | 5-10-5 | HBV | HBV-related disease | | | |
| AUS1648 | 609 | GCGCCgacgggacgtAAACA | 5-10-5 | HBV | HBV-related disease | | | |
| AUS1649 | 610 | CGCCGacgggacgtaAACAA | 5-10-5 | HBV | HBV-related disease | | | |
| AUS1650 | 611 | GCCGAcgggacgtaaACAAA | 5-10-5 | HBV | HBV-related disease | | | |
| AUS1651 | 612 | CCGACgggacgtaaaCAAAG | 5-10-5 | HBV | HBV-related disease | | | |
| AUS1652 | 613 | CGACGggacgtaaacAAAGG | 5-10-5 | HBV | HBV-related disease | | | |
| AUS1653 | 614 | GCGAAgtgcacacggTCCGGAACAC | 5-10-25 | HBV | HBV-related disease | 0.45 | | 0.00 |
| | | AACACAACACAACAC | | | | | | |
| AUS1654 | 615 | AACACAACACGCGAAgtgcacacgg | 15-10-15 | HBV | HBV-related disease | 0.67 | | 0.00 |
| | | TCCGGAACACAACAC | | | | | | |
| AUS1655 | 616 | AACACAACACAACACAACACGCGAA | 25-10-5 | HBV | HBV-related disease | 0.43 | | 0.00 |
| | | gtgcacacggTCCGG | | | | | | |
| AUS1656 | 617 | CACTAgtaaactgagCCAGGAACAC | 5-10-25 | HBV | HBV-related disease | 1.14 | 0.29 | 0.26 |
| | | AACACAACACAACAC | | | | | | |
| AUS1657 | 618 | TCCGCagtatggatcGGCAGAACAC | 5-10-25 | HBV | HBV-related disease | 0.44 | | 0.00 |
| | | AACACAACACAACAC | | | | | | |
| AUS1658 | 619 | CTGCGgtattgtgagGATTCAACAC | 5-10-25 | HBV | HBV-related disease | 0.78 | | 0.00 |
| | | AACACAACACAACAC | | | | | | |
| AUS1659 | 620 | GCAGAggtgaagcgaAGTGCAACAA | 5-10-25 | HBV | HBV-related disease | 0.87 | | 0.00 |
| | | CAACAACAACAACAA | | | | | | |
| AUS1660 | 621 | GCGAAgtgcacacggACCGG | 5-10-5 | HBV | HBV-related disease | 0.63 | | 0.00 |
| AUS1661 | 622 | CGGTCcggcagatgaGAAGG | 5-10-5 | HBV | HBV-related disease | | | |
| AUS1662 | 623 | GTCCGgcagatgagaAGGCA | 5-10-5 | HBV | HBV-related disease | | | |
| AUS1663 | 624 | CCGGCagatgagaagGCACA | 5-10-5 | HBV | HBV-related disease | | | |
| AUS1664 | 625 | GGCAGatgagaaggcACAGA | 5-10-5 | HBV | HBV-related disease | | | |
| AUS1665 | 626 | AAGGCacagacggggAGTCC | 5-10-5 | HBV | HBV-related disease | | | |
| AUS1666 | 627 | GGCACagacggggagTCCGC | 5-10-5 | HBV | HBV-related disease | | | |
| AUS1667 | 628 | CACAGacggggagtcCGCGT | 5-10-5 | HBV | HBV-related disease | | | |
| AUS1668 | 629 | GAGTCcgcgtaaagaGAGGT | 5-10-5 | HBV | HBV-related disease | | | |
| AUS1669 | 630 | AGTCCgcgtaaagagAGGTG | 5-10-5 | HBV | HBV-related disease | | | |
| AUS1670 | 631 | GTCCGcgtaaagagaGGTGC | 5-10-5 | HBV | HBV-related disease | 1.12 | 3.19 | 2.84 |
| AUS1671 | 632 | TCCGCgtaaagagagGTGCG | 5-10-5 | HBV | HBV-related disease | | | |
| AUS1672 | 633 | CCGCGtaaagagaggTGCGC | 5-10-5 | HBV | HBV-related disease | | | |
| AUS1673 | 634 | GCGTAaagagaggtgCGCCC | 5-10-5 | HBV | HBV-related disease | | | |
| AUS1674 | 635 | GTAAAgagaggtgcgCCCCG | 5-10-5 | HBV | HBV-related disease | | | |
| AUS1675 | 636 | AAAGAgaggtgcgccCCGTG | 5-10-5 | HBV | HBV-related disease | | | |
| AUS1676 | 637 | GCGAAgtgcacacggTCCGG | 5-10-5 | HBV | HBV-related disease | | | |
| AUS1678 | 638 | GTCCGcgtaaagagaGGTGC | 5-10-5 | HBV | HBV-related disease | | | |
| AUS1679 | 639 | GTGAAgcgaagtgcaCACGG | 5-10-5 | HBV | HBV-related disease | 1.4 | >250 | |
| AUS1681 | 640 | CCTTCcctgaaggttCCTCC | 5-10-5 | HBV | HBV-related disease | | | |
| AUS1683 | 641 | GGTCCggcagatgagAAGGC | 5-10-5 | HBV | HBV-related disease | 1.23 | 0.78 | 0.63 |
| AUS1684 | 642 | GTCCGcgtaaagagaGGTGC | 5-10-5 | HBV | HBV-related disease | 2.74 | 2.19 | 0.80 |
| AUS1685 | 643 | GTGAAgcgaagtgcaCACGG | 5-10-5 | HBV | HBV-related disease | 2.66 | 0.98 | 0.37 |
| AUS1687 | 644 | UGACUgtgaacgttcgaGAUGA | 5-12-5 | HBV | HBV-related disease | | | |
| AUS1688 | 645 | UGACUgtgaacgttcgaGAUGA | 5-12-5 | HBV | HBV-related disease | | | |
| AUS1689 | 646 | UGACUgtgaacgttcgaGAUGA | 5-12-5 | HBV | HBV-related disease | | | |
| AUS1690 | 647 | UGACUgtgaacgttcga+G+A+T+G | 5-12-5 | HBV | HBV-related disease | | | |
| | | +A | | | | | | |
| AUS1691 | 648 | +T+G+A+C+Tgtgaacgttcga+G+ | 5-12-5 | HBV | HBV-related disease | | | |
| | | A+T+G+A | | | | | | |
| AUS1693 | 649 | CCGGCagatgagaagGCACA | 5-10-5 | HBV | HBV-related disease | | | |
| AUS1694 | 650 | GCGAAgtgcacacggTCCGG | 5-10-5 | HBV | HBV-related disease | | | |
| AUS1695 | 651 | GTCCGcgtaaagagaGGTGC | 5-10-5 | HBV | HBV-related disease | | | |
| AUS1696 | 652 | GTGAAgcgaagtgcaCACGG | 5-10-5 | HBV | HBV-related disease | | | |
| AUS1591 | 552 | GCACTAGTAAACTGAGCCAG | n/a | HBV | HBV-related disease | >250 | 5.82 | |

Conjugation

| | | | | | | | |
|---|---|---|---|---|---|---|---|
| AUS1704 | 658 | GCAGaggtgAagcgaA+GT+G+C | 4-5-1-5-5 | HBV | HBV-related disease | | |
| AUS1705 | 659 | GCAGaggtgAagcgaA+GT+G+C | 4-5-1-5-5 | HBV | HBV-related disease | 5.7 | 0.3 |
| AUS1703 | 657 | GCAGAggtgaagcgaAGTGC | 5-10-5 | HBV | HBV-related disease | | |

*Motif shows the regions of each sequence from 5′ to 3′ (e.g., W1-G1-W2 or W1-G1-S1-G2-W2), where the separator (S1) position is shown in bolded font.

TABLE 1B
Inhibition of PCSK9, DMPK, TTR, or AAT expression in cells
using modified multi-segmented antisense oligonucleotides

| SEQ | | | | | | | Maximum | |
|---|---|---|---|---|---|---|---|---|
| ID | | | | | EC₂₅ | EC₅₀ | Inhibi- | Tm |
| NO: | Sequence | Motif* | Target | Indication | (nM) | (nM) | tion % | (° C.) |
| 668 | GGGCtcataGcacatTATCC | 4-5-1-5-5 | PCSK9 | HeFH/Hyperlipidemia | 0.94 | 258.1 | 59 | 64.00 |
| 670 | GGGCtcataGcacatT + | 4-5-1-5-5 | PCSK9 | HeFH/Hyperlipidemia | 2.45 | / | 50 | |
| | A + TCC | | | | | | | |
| 673 | GGGCtcataGcacatT + | 4-5-1-5-5 | PCSK9 | HeFH/Hyperlipidemia | 2.62 | 13.3 | 66 | |
| | AT + CC | | | | | | | |
| 674 | GGGCtcataGcacatT + | 4-5-1-5-5 | PCSK9 | HeFH/Hyperlipidemia | 8.5 | 59.02 | 30.1 | |
| | ATC + C | | | | | | | |
| 669 | GGGCTcataGcacatTATCC | 5-4-1-5-5 | PCSK9 | HeFH/Hyperlipidemia | 6.19 | 33.9 | 51 | |
| 671 | GGGCTcataGcacatT + | 5-4-1-5-5 | PCSK9 | HeFH/Hyperlipidemia | 0.36 | 1.92 | 82 | |
| | A + TCC | | | | | | | |
| 672 | GGGCTcatagCacatTATCC | 5-5-1-4-5 | PCSK9 | HeFH/Hyperlipidemia | 1.27 | 6.17 | 77 | |
| 675 | GGGCTcatagCacatT + | 5-5-1-4-5 | PCSK9 | HeFH/Hyperlipidemia | 1.836 | 8.62 | 85.3 | |
| | A + TCC | | | | | | | |
| 676 | GGGCTcataGcacatT + | 5-4-1-5-5 | PCSK9 | HeFH/Hyperlipidemia | 1.678 | 6.46 | 68 | |
| | ATC + C | | | | | | | |
| 677 | GGGCTcatagCacatT + | 5-4-1-5-5 | PCSK9 | HeFH/Hyperlipidemia | 17.3 | 47.9 | 33 | |
| | ATC + C | | | | | | | |
| 667 | GGGCTcatagcacatTATCC | 5-10-5 | PCSK9 | HeFH/Hyperlipidemia | 4.51 | 80.1 | 32 | 67.00 |
| 679 | ACAGacaatAaatacCGAGG | 4-5-1-5-5 | DMPK | Myotonic Dystrophy 1 | 32.8 | / | 31.6 | 59.00 |
| 681 | ACAGacaatAaatacC + | 4-5-1-5-5 | DMPK | Myotonic Dystrophy 1 | 45.3 | / | 42.3 | |
| | G + AGG | | | | | | | |
| 686 | ACAGacaatAaatacC + | 4-5-1-5-5 | DMPK | Myotonic Dystrophy 1 | 6.7 | 36 | 42.2 | |
| | GA + GG | | | | | | | |
| 687 | ACAGacaatAaatacC + | 4-5-1-5-5 | DMPK | Myotonic Dystrophy 1 | 0.5 | 3.4 | 73.6 | |
| | GAG + G | | | | | | | |
| 680 | ACAGAcaatAaatacCGAGG | 5-4-1-5-5 | DMPK | Myotonic Dystrophy 1 | 16.2 | 30.9 | 45.4 | |
| 682 | ACAGAcaatAaatacC + | 5-4-1-5-5 | DMPK | Myotonic Dystrophy 1 | 6.5 | 101.9 | 42.5 | |
| | G + AGG | | | | | | | |
| 685 | ACAGAcaataAatacCGAGG | 5-5-1-4-5 | DMPK | Myotonic Dystrophy 1 | 17.8 | 70.2 | 41.7 | |
| 688 | ACAGAcaataAatacC + | 5-5-1-4-5 | DMPK | Myotonic Dystrophy 1 | 2.7 | 58.8 | 48.1 | |
| | G + AGG | | | | | | | |
| 678 | ACAGAcaataaatacCGAGG | 5-10-5 | DMPK | Myotonic Dystrophy 1 | 40 | 96.4 | 21.3 | 60.00 |
| 683 | ACAGacaataaatacCGAGG | 4-11-5 | DMPK | Myotonic Dystrophy 1 | 12.6 | 60 | 34 | |
| 684 | ACAGacaataAatacCGAGG | 4-6-1-4-5 | DMPK | Myotonic Dystrophy 1 | 34.6 | 160.2 | 24.8 | |
| 691 | TACAaatggGatgctACTGC | 4-5-1-5-5 | TTR | hATTR Amyloidosis- | 4.6 | 11.1 | 96.40% | 60.50 |
| | | | | Polyneuropathy/ATTR | | | | |
| | | | | Amyloidosis-CM | | | | |
| 692 | TACAaatggGatgctA + | 4-5-1-5-5 | TTR | hATTR Amyloidosis- | 4.6 | 14.3 | 73.20% | |
| | C + TGC | | | Polyneuropathy/ATTR | | | | |
| | | | | Amyloidosis-CM | | | | |
| 690 | TACAAatggGatgctACTGC | 5-4-1-5-5 | TTR | hATTR Amyloidosis- | 4 | 426.5 | 47.70% | |
| | | | | Polyneuropathy/ATTR | | | | |
| | | | | Amyloidosis-CM | | | | |
| 689 | TACAAatgggatgctACTGC | 5-10-5 | TTR | hATTR Amyloidosis- | 3.4 | 10.6 | 82.70% | 61.50 |
| | | | | Polyneuropathy/ATTR | | | | |
| | | | | Amyloidosis-CM | | | | |
| 693 | ACACAgtttgaatgaAATAG | 5-10-5 | TTR | hATTR Amyloidosis- | 25.4 | 153.3 | 41.40% | |
| | | | | Polyneuropathy/ATTR | | | | |
| | | | | Amyloidosis-CM | | | | |
| 694 | ACAGTttgaatgaaaTAGCA | 5-10-5 | TTR | hATTR Amyloidosis- | 3.9 | 20.2 | 91.60% | |
| | | | | Polyneuropathy/ATTR | | | | |
| | | | | Amyloidosis-CM | | | | |
| 695 | AGTTTgaatgaaataGCAGG | 5-10-5 | TTR | hATTR Amyloidosis- | 16.7 | 36.2 | 69.30% | |
| | | | | Polyneuropathy/ATTR | | | | |
| | | | | Amyloidosis-CM | | | | |
| 696 | TTTGAatgaaatagcAGGTG | 5-10-5 | TTR | hATTR Amyloidosis- | 0.4 | 17.7 | 61.30% | |
| | | | | Polyneuropathy/ATTR | | | | |
| | | | | Amyloidosis-CM | | | | |
| 699 | ACCCaattcAgaaggAAGGA | 4-5-1-5-5 | AAT | α-1 Antitrypsin | 1.6 | 3.2 | 85.3 | 62.00 |
| | | | | Deficiency | | | | |
| 700 | ACCCaattcAgaaggA + | 4-5-1-5-5 | AAT | α-1 Antitrypsin | 4.5 | 3.5 | 60.3 | |
| | A + GGA | | | Deficiency | | | | |
| 698 | ACCCAattcAgaaggAAGGA | 5-4-1-5-5 | AAT | α-1 Antitrypsin | 5.1 | 8.9 | 65 | |
| | | | | Deficiency | | | | |
| 697 | ACCCAattcagaaggAAGGA | 5-10-5 | AAT | α-1 Antitrypsin | 0.6 | 5.6 | 87.2 | 64.50 |
| | | | | Deficiency | | | | |
| 843 | TCGAagacaGttctaGGGTT | 4-5-1-5-5 | DMPK | Myotonic Dystrophy 1 | | 33.6 | 94.7 | 63.00 |
| 846 | CTTCccgaaTgtccgACAGT | 4-5-1-5-5 | DMPK | Myotonic Dystrophy 1 | | 3.67 | 100 | 68.50 |
| 847 | CCCAgcgccCaccagTCACA | 4-5-1-5-5 | DMPK | Myotonic Dystrophy 1 | | 1.82 | 100 | 76.50 |
| 849 | CCCAGcgccCaccagTCACA | 5-4-1-5-5 | DMPK | Myotonic Dystrophy 1 | | 1.7 | 100 | |
| 846 | CTTCCcgaaTgtccgACAGT | 5-4-1-5-5 | DMPK | Myotonic Dystrophy 1 | | 6.84 | 98.8 | |
| 844 | TCGAAgacagttctaGGGTT | 5-10-5 | DMPK | Myotonic Dystrophy 1 | | 33.5 | 94.8 | 65.00 |
| 845 | CTTCCcgaatgtccgACAGT | 5-10-5 | DMPK | Myotonic Dystrophy 1 | | 2.57 | 100 | 68.00 |
| 848 | CCCAGcgcccaccagTCACA | 5-10-5 | DMPK | Myotonic Dystrophy 1 | | 1.33 | 100 | 79.50 |

*Motif shows the regions of each sequence from 5′ to 3′ (e.g., W1-G1-W2 or W1-G1-S1-G2-W2), where the separator (S1) position is shown in bolded font.

TABLE 1C
Miscellaneous modified multi-segmented antisense oligonucleotides sequences

| SEQ | | | | |
|---|---|---|---|---|
| ID | | | | |
| NO: | Sequence* | Motif† | Target | Indication |
| 851 | AAGAucuggAcaaaaCGAAA | 4-5-1-5-5 | Proprotein convertase | High cardiovascular risk and |
| | | | subtilisin/kexin type 12 | elevated LDL-cholesterol |
| 852 | AGAUcuggaCaaaacGAAAA | 4-5-1-5-5 | Proprotein convertase | High cardiovascular risk and |
| | | | subtilisin/kexin type 12 | elevated LDL-cholesterol |
| 853 | GAUCuggacAaaacgAAAAC | 4-5-1-5-5 | Proprotein convertase | High cardiovascular risk and |
| | | | subtilisin/kexin type 12 | elevated LDL-cholesterol |
| 854 | AUCUggacaAaacgaAAACA | 4-5-1-5-5 | Proprotein convertase | High cardiovascular risk and |
| | | | subtilisin/kexin type 12 | elevated LDL-cholesterol |
| 867 | UGCUccgttGgtgctUGUUC | 4-5-1-5-5 | Apo(a) | Cardiovascular disease |
| 868 | AGCUucttgTccagcUUUAU | 4-5-1-5-5 | APOCIII | Cardiovascular disease |
| 869 | UCUUggttaCatgaaAUCCC | 4-5-1-5-5 | Transthyretin mRNA | hATTR Amyloidosis- |
| | | | | Polyneuropathy |
| 872 | GCCUcagtcTgcttcGCACC | 4-5-1-5-5 | ApoB mRNA | Familial hypercholesterolemia |
| 880 | CUUGguuacAugaaaUCCCA | 4-5-1-5-5 | Transthyretin | hATTR |
| 881 | UUGGuuacaUgaaauCCCAU | 4-5-1-5-5 | Transthyretin | hATTR |
| 882 | UGGUuacauGaaaucCCAUC | 4-5-1-5-5 | Transthyretin | hATTR |
| 883 | CACAaacaaGctggtCGGTT | 4-5-1-5-5 | Angiotensinogen | Treatment-Resistant |
| | | | | Hypertension/Reduced Ejection |
| | | | | Fraction |
| 894 | UUAAgaagaCaaaggGUUUG | 4-5-1-5-5 | SERPINA1 gene/alpha-1 | α-1 Antitrypsin Deficiency |
| | | | antitrypsin | |
| 895 | UAAGaagacAaagggUUUGG | 4-5-1-5-5 | SERPINA1 gene/alpha-1 | α-1 Antitrypsin Deficiency |
| | | | antitrypsin | |
| 896 | GUUAaacauGccuaaACGCU | 4-5-1-5-5 | Mutant of α1-antitrypsin | α-1 Antitrypsin Deficiency |
| | | | (Z-AAT) | |
| 897 | AGAAagaguGucucaUCUUA | 4-5-1-5-5 | ALAS1 mRNA | Acute hepatic porphyria |
| 898 | GGUAgcccuUgggcuCGUGG | 4-5-1-5-5 | TGFB1 | Inflammation and injury |
| 899 | GUAGcccuuGggcucGUGGA | 4-5-1-5-5 | TGFB1 | Inflammation and injury |
| 900 | UAGCccuugGgcucgUGGAU | 4-5-1-5-5 | TGFB1 | Inflammation and injury |
| 901 | AGCCcuuggGcucguGGAUC | 4-5-1-5-5 | TGFB1 | Inflammation and injury |
| 902 | GCCCuugggCucgugGAUCC | 4-5-1-5-5 | TGFB1 | Inflammation and injury |
| 903 | CAUCaucagAccaggCACCA | 4-5-1-5-5 | TGFB1 AND Cox2 | COVID-19 |
| 904 | AUCAucagaCcaggcACCAG | 4-5-1-5-5 | TGFB1 AND Cox2 | COVID-19 |
| 905 | UCAUcagacCaggcaCCAGA | 4-5-1-5-5 | TGFB1 AND Cox2 | COVID-19 |
| 906 | CAUCagaccAggcacCAGAC | 4-5-1-5-5 | TGFB1 AND Cox2 | COVID-19 |
| 907 | AUCAgaccaGgcaccAGACC | 4-5-1-5-5 | TGFB1 AND Cox2 | COVID-19 |
| 908 | TGCCatttaAtgagcTTCAC | 4-5-1-5-5 | DGAT2 | Nonalcoholic fatty liver |
| | | | | disease |
| 909 | ACCAgugacAaugacCGCUU | 4-5-1-5-5 | | |
| 910 | UGUCcaggaAguccuCAAGU | 4-5-1-5-5 | Protein kinase N3 | Pancreatic cancer |
| 911 | GUCCaggaaGuccucAAGUC | 4-5-1-5-5 | Protein kinase N3 | Pancreatic cancer |
| 912 | UCCAggaagUccucaAGUCU | 4-5-1-5-5 | Protein kinase N3 | Pancreatic cancer |
| 913 | AGCUcaucuCuccuaUGUGC | 4-5-1-5-5 | Kinesin spindle protein and | Solid tumor |
| | | | vascular endothelial growth | |
| | | | factor | |
| 914 | GCUCaucucUccuauGUGCU | 4-5-1-5-5 | Kinesin spindle protein and | Solid tumor |
| | | | vascular endothelial growth | |
| | | | factor | |
| 915 | CUCAucucuCcuaugUGCUG | 4-5-1-5-5 | Kinesin spindle protein and | Solid tumor |
| | | | vascular endothelial growth | |
| | | | factor | |
| 916 | GUUAguuuaGauucuCGATT | 4-5-1-5-5 | Kinesin spindle protein and | Solid tumor |
| | | | vascular endothelial growth | |
| | | | factor | |
| 917 | UAUUgugagGauuuuUGUCG | 4-5-1-5-5 | Hepatitis B surface antigen | Chronic hepatitis B viral |
| | | | | infection |
| 918 | AUUGugaggAuuuuuGUCGG | 4-5-1-5-5 | Hepatitis B surface antigen | Chronic hepatitis B viral |
| | | | | infection |
| 919 | ACCAauuuaUgccuaCAGCG | 4-5-1-5-5 | Hepatitis B surface antigen | Chronic hepatitis B viral |
| | | | | infection |
| 920 | GAAAauugaGagaagUCCAC | 4-5-1-5-5 | | |
| 921 | CGTTtgctcTtcttcTTGCG | 4-5-1-5-5 | IE2 mRNA | CMV retinitis |
| 927 | GAAGuuggcAugguaGCCCU | 4-5-1-5-5 | TGF-β1/COX-2 | Keloid scarless healing |
| 928 | AAGUuggcaUgguagCCCUU | 4-5-1-5-5 | TGF-β1/COX-2 | Keloid scarless healing |
| 929 | AGUUggcauGguagcCCUUG | 4-5-1-5-5 | TGF-β1/COX-2 | Keloid scarless healing |
| 930 | GUUGgcaugGuagccCUUGG | 4-5-1-5-5 | TGF-β1/COX-2 | Keloid scarless healing |
| 931 | UUGGcauggUagcccUUGGG | 4-5-1-5-5 | TGF-β1/COX-2 | Keloid scarless healing |
| 934 | GCAGaggtgAagcgaAGUGC | 4-5-1-5-5 | Hepatitis B viral proteins | Hepatitis B viral infection |
| | | | (including surface antigen) | |
| 935 | GUGAagcgaAgugcaCACUU | 4-5-1-5-5 | Hepatitis B surface antigen | Hepatitis B viral infection |
| 936 | GUGAagcgaAgugcaCACGG | 4-5-1-5-5 | Hepatitis B surface antigen | Hepatitis B viral infection |
| 937 | UGAAgcgaaGugcacACGGU | 4-5-1-5-5 | Hepatitis B surface antigen | Hepatitis B viral infection |
| 938 | CAGUautacCtctacTAGUC | 4-5-1-5-5 | GFAP | Alexander Disease |
| 939 | CTTCacattTcgatcCAACA | 4-5-1-5-5 | ATXN2 | Amyotrophic Lateral Sclerosis |
| 940 | GCAAugtcaCctttcAUACC | 4-5-1-5-5 | FUS | Amyotrophic Lateral Sclerosis |
| 941 | CAGGatacaTttctaCAGCU | 4-5-1-5-5 | SOD1 | Amyotrophic Lateral Sclerosis |
| 942 | TTGCctccgGttctgAAGGT | 4-5-1-5-5 | Exon 53 Dystrophin | Duchenne muscular dystrophy |
| 943 | TGCCtccggTtctgaAGGTG | 4-5-1-5-5 | Exon 53 Dystrophin | Duchenne muscular dystrophy |
| 944 | GCCTccggtTctgaaGGTGT | 4-5-1-5-5 | Exon 53 Dystrophin | Duchenne muscular dystrophy |
| 945 | CCTCcggttCtgaagGTGTT | 4-5-1-5-5 | Exon 53 Dystrophin | Duchenne muscular dystrophy |
| 946 | CTCCggttcTgaaggTGTTC | 4-5-1-5-5 | Exon 53 Dystrophin | Duchenne muscular dystrophy |
| 947 | AATGccatcCtggagTTCCT | 4-5-1-5-5 | Exon 45 Dystrophin | Duchenne Muscular Dystrophy |
| | | | | (DMD) |
| 948 | ATGCcatccTggagtTCCTG | 4-5-1-5-5 | Exon 45 Dystrophin | Duchenne Muscular Dystrophy |
| | | | | (DMD) |
| 951 | UGGAauacuCuugguUACTT | 4-5-1-5-5 | Transthyretin mRNA | hATTR Amyloidosis- |
| | | | | Polyneuropathy |
| 954 | CUCAgtaacAttgacACCAC | 4-5-1-5-5 | HTT | Huntington′s Disease |
| 955 | UCGAagacaGuucuaGGGUU | 4-5-1-5-5 | DMPK | Myotonic Dystrophy Type 1 |
| | | | | (DM1) |
| 957 | GGAAucaguGaaugcUUAUA | 4-5-1-5-5 | VEGF protein | Age-related macular |
| | | | | degeneration |
| 958 | GAAUcagugAaugcuUAUAC | 4-5-1-5-5 | VEGF protein | Age-related macular |
| | | | | degeneration |
| 959 | AAUCagugaAugcuuAUACA | 4-5-1-5-5 | VEGF protein | Age-related macular |
| | | | | degeneration |
| 960 | AUCAgugaaUgcuuaUACAU | 4-5-1-5-5 | VEGF protein | Age-related macular |
| | | | | degeneration |
| 961 | UCAGugaauGcuuauACAUC | 4-5-1-5-5 | VEGF protein | Age-related macular |
| | | | | degeneration |
| 962 | CAGUgaaugCuuauaCAUCC | 4-5-1-5-5 | VEGF protein | Age-related macular |
| | | | | degeneration |
| 963 | AGUGaaugcUuauacAUCCG | 4-5-1-5-5 | VEGF protein | Age-related macular |
| | | | | degeneration |
| 964 | GUGAaugcuUauacaUCCGT | 4-5-1-5-5 | VEGF protein | Age-related macular |
| | | | | degeneration |
| 965 | AUCCcacgcCcctgtCCAGC | 4-5-1-5-5 | Complement Factor B | IGA nephropathy |
| 966 | UGGAucacaUgcacaAUGTT | 4-5-1-5-5 | β2-Adrenergic receptor | Open angle glaucoma/Ocular |
| | | | | hypertension |
| 967 | GCUUcggagAaauuuAAAUC | 4-5-1-5-5 | Usherin (exon 13 of USH2A) | Retinitis pigmentosa |
| 969 | AGCAagauaUuuuuaUAAUA | 4-5-1-5-5 | Complement component C5 | IgA nephropathy/Berger |
| | | | | disease |
| 973 | AUAGgaaacAccaaaGAUGA | 4-5-1-5-5 | Cystic fibrosis transmembrane | Cystic fibrosis |
| | | | conductance regulator | |
| | | | (ΔF508 mutation) | |
| 974 | GGAAacaccAaagauGAUAU | 4-5-1-5-5 | Cystic fibrosis transmembrane | Cystic fibrosis |
| | | | conductance regulator | |
| | | | (ΔF508 mutation) | |
| 975 | AACAccaaaGaugauAUUUU | 4-5-1-5-5 | Cystic fibrosis transmembrane | Cystic fibrosis |
| | | | conductance regulator | |
| | | | (ΔF508 mutation) | |
| 976 | ACCAaagauGauauuUUCUU | 4-5-1-5-5 | Cystic fibrosis transmembrane | Cystic fibrosis |
| | | | conductance regulator | |
| | | | (ΔF508 mutation) | |
| 980 | GCTTagagtAcagccCACTT | 4-5-1-5-5 | TMPRSS6 | Beta Thalassemia |
| 981 | GGUUaacacCauuuaCUUCA | 4-5-1-5-5 | Antithrombin | Hemophilia |
| 982 | ACGGcattgGtgcacAGUUU | 4-5-1-5-5 | Factor XI | Thrombotic Disorder |
| 987 | AGCAccauuUgaaauCAGUG | 4-5-1-5-5 | miR-29 | Keloid |
| 988 | GCACcauuuGaaaucAGUGU | 4-5-1-5-5 | miR-29 | Keloid |
| 989 | CACCauuugAaaucaGUGUU | 4-5-1-5-5 | miR-29 | Keloid |
| 990 | ACCAuuugaAaucagUGUUU | 4-5-1-5-5 | miR-29 | Keloid |
| 991 | CCAUuugaaAucaguGUUUU | 4-5-1-5-5 | miR-29 | Keloid |
| 997 | ACACaucugGcuguuCCAGC | 4-5-1-5-5 | HOXB13 | Inflammation and injury |
| 998 | CACAucuggCuguucCAGCC | 4-5-1-5-5 | HOXB13 | Inflammation and injury |
| 999 | ACAUcuggcUguuccAGCCA | 4-5-1-5-5 | HOXB13 | Inflammation and injury |
| 1000 | CAUCuggcuGuuccaGCCAC | 4-5-1-5-5 | HOXB13 | Inflammation and injury |
| 1001 | AUCUggcugUuccagCCACC | 4-5-1-5-5 | HOXB13 | Inflammation and injury |
| 1002 | UGAAauguuUaggacCACUA | 4-5-1-5-5 | Vascular endothelial growth | Ulcers associated with type |
| | | | factor-A (VEGF-A) | II diabetes |
| 1003 | GAAAuguuuAggaccACUAG | 4-5-1-5-5 | Vascular endothelial growth | Ulcers associated with type |
| | | | factor-A (VEGF-A) | II diabetes |
| 1004 | GCTGattagAgagagGTCCC | 4-5-1-5-5 | TNFα | Autoimmune |
| 1006 | GCCCaagctGgcatcCGTCA | 4-5-1-5-5 | ICAM-1 inhibitor | Moderate to severe |
| | | | | Inflammatory Bowel Disease |
| | | | | (IBD) |
| 1007 | CCACctttgGgtgaaTAGCA | 4-5-1-5-5 | GHr | Acromegaly |
| 1008 | TGCAagtctCttggcAAACA | 4-5-1-5-5 | PKK | Hereditary Angioedema |
| 1009 | ATCCggaggGctcgcCATGC | 4-5-1-5-5 | INSR inhibitor | keratitis-related progressive |
| | | | | corneal neovascularization |
| 1010 | TCCGgagggCtcgccATGCT | 4-5-1-5-5 | INSR inhibitor | keratitis-related progressive |
| | | | | corneal neovascularization |
| 1011 | CCGGagggcTcgccaTGCTG | 4-5-1-5-5 | INSR inhibitor | keratitis-related progressive |
| | | | | corneal neovascularization |
| 1012 | CGGAgggctCgccatGCTGC | 4-5-1-5-5 | INSR inhibitor | keratitis-related progressive |
| | | | | corneal neovascularization |
| 1013 | GGAGggctcGccatgCTGCT | 4-5-1-5-5 | INSR inhibitor | keratitis-related progressive |
| | | | | corneal neovascularization |
| 1016 | CTGAgtctgTtttccATTCT | 4-5-1-5-5 | CD49d antagonists | Relapsing-remitting multiple |
| | | | | sclerosis, acute myeloid |
| | | | | leukemia, asthma and Duchenne |
| | | | | muscular dystrophy and limb |
| | | | | girdle muscular dystrophy R2 |
| 1017 | TGTGtctttCttctgTTAGC | 4-5-1-5-5 | Exon44 | Duchenne muscular dystrophy |
| 1018 | GTGTctttcTtctgtTAGCC | 4-5-1-5-5 | Exon44 | Duchenne muscular dystrophy |
| 1019 | TGTCtttctTctgttAGCCA | 4-5-1-5-5 | Exon44 | Duchenne muscular dystrophy |
| 1020 | GTCTttcttCtgttaGCCAC | 4-5-1-5-5 | Exon44 | Duchenne muscular dystrophy |
| 1027 | AAACccuuuGucuucUUAA | 4-5-1-5-4 | Mutant of α-1 antitrypsin | ZZ Type α-1 Antitrypsin |
| | | | | deficiency liver disease |
| 1028 | UGUUaaacaUgccuaAACGC | 4-5-1-5-6 | Mutant of α1-antitrypsin | α-1 Antitrypsin Deficiency |
| | U | | (Z-AAT) | |
| 1029 | CAGAaagagUgucucAUCUU | 4-5-1-5-6 | ALAS1 mRNA | Acute hepatic porphyria |
| | A | | | |
| 1031 | GATtttgaCattctGC | 3-5-1-5-2 | FOXP3 | Chronic Kidney Disease |
| 1033 | GACCagugaCaaugaCCGCU | 4-5-1-5-6 | MTL-CEBPA | 2L advanced liver cancer |
| | U | | | |
| 1035 | AGUUaguuuAgauucUCGAT | 4-5-1-5-6 | Kinesin spindle protein and | Solid tumor |
| | T | | vascular endothelial growth | |
| | | | factor | |
| 1037 | UACCaauuuAugccuACAGC | 4-5-1-5-6 | Hepatitis B surface antigen | Chronic hepatitis B viral |
| | G | | | infection |
| 1039 | GCGTttgctCttcttCTTGC | 4-5-1-5-6 | IE2 mRNA | CMV retinitis |
| | G | | | |
| 1040 | UUUUaggcuCuguugGUGGT | 4-5-1-5-6 | COVID-19 | COVID-19 |
| | T | | | |
| 1041 | UUCAacagaAcuuccUUCCT | 4-5-1-5-6 | COVID-19 | COVID-19 |
| | T | | | |
| 1042 | UGUGaagcgAagugcACACU | 4-5-1-5-6 | Hepatitis B surface antigen | Hepatitis B viral infection |
| | U | | | |
| 1043 | CCGTtttctTaccacCCT | 4-5-1-5-3 | TAU | Alzheimer′s Disease & |
| | | | | Frontotemporal Degeneration |
| 1045 | GGCataagGtcacgGA | 3-5-1-5-2 | DNM2 | Centronuclear Myopathy |
| 1046 | CCTCcggttCtgaagGTGTT | 4-5-1-5-6 | Exon 53 Dystrophin | Duchenne muscular dystrophy |
| | C | | | |
| 1047 | AUGGaauacUcuuggUUACT | 4-5-1-5-6 | Transthyretin mRNA | hATTR Amyloidosis- |
| | T | | | Polyneuropathy |
| 1052 | UCACuuucaUaaugcUGG | 4-5-1-5-3 | SMN2 mRNA | Spinal muscular atrophy |
| 1053 | CAGcgcccAccaguCA | 3-5-1-5-2 | DMPK | Myotonic Dystrophy 1 |
| 1054 | AGGAguuccAcauucUGGC | 4-5-1-5-4 | Caspase 2 | Acute primary angle closure |
| | | | | glaucoma |
| 1055 | AGCUgcaucAgguugGCAC | 4-5-1-5-4 | RTP801 | Age-related macular |
| | | | | degeneration |
| 1057 | UGAAguagaAgaugcGCUU | 4-5-1-5-4 | Transient receptor potential | Dry eye disease |
| | | | cation channel subfamily V | |
| | | | Member 1 | |
| 1058 | GGUggaucAcgaguUCA | 3-5-1-5-3 | Centrosomal protein 290 | Leber′s Congenital amaurosis |
| 1059 | CUGGaucacAugcacAAUGT | 4-5-1-5-6 | β2-Adrenergic receptor | Open angle glaucoma/Ocular |
| | T | | | hypertension |
| 1060 | AGCUucggaGaaauuUAAAU | 4-5-1-5-6 | Usherin (exon 13 of USH2A) | Retinitis pigmentosa |
| | C | | | |
| 1061 | ACATcagtcTgauaaGCTA | 4-5-1-5-4 | miR-21 | Alport′s syndrome |
| 1063 | TGGGagtagAtgaggTAC | 4-5-1-5-3 | TNFα | Autoimmune |
| 1066 | CGGCatgtcTattttGTA | 4-5-1-5-3 | TGF-β2 inhibitor | Inflammation and injury |
| 1067 | AATgctacAaaaccCA | 3-5-1-5-2 | PCSK9 | Hypercholesterolemia |
| 1068 | GAccagAtgcagGA | 2-4-1-5-2 | TGF-β2 inhibitor | Inflammation and injury |
| 1071 | AGUUggagcAagauuAUC | 4-5-1-5-3 | Nav1.1 | Dravet Syndrome |
| 1072 | AAGAucuggAcaaaaCGAAA | 4-5-1-5-8 | Proprotein convertase | High cardiovascular risk and |
| | ACA | | subtilisin/kexin type 12 | elevated LDL-cholesterol |
| 1077 | UCUUgguuaCaugaaAUCCC | 4-5-1-5-8 | Transthyretin | hATTR |
| | AUC | | | |
| 1079 | UUUAagaagAcaaagGGUUU | 4-5-1-5-7 | SERPINA1 gene/alpha-1 | α-1 Antitrypsin Deficiency |
| | GG | | antitrypsin | |
| 1080 | UUGUccaggAaguccUCAAG | 4-5-1-5-8 | Protein kinase N3 | Pancreatic cancer |
| | UCU | | | |
| 1081 | AAGCucaucUcuccuAUGUG | 4-5-1-5-8 | Kinesin spindle protein and | Solid tumor |
| | CUG | | vascular endothelial growth | |
| | | | factor | |
| 1082 | UUAUugugaGgauuuUUGUC | 4-5-1-5-7 | Hepatitis B surface antigen | Chronic hepatitis B viral |
| | GG | | | infection |
| 1083 | UGUGaagcgAagugcACACG | 4-5-1-5-7 | Hepatitis B surface antigen | Chronic hepatitis B viral |
| | GU | | | infection |
| 1084 | CAATgccatCctggaGTTCC | 4-5-1-5-7 | Exon 45 Dystrophin | Duchenne Muscular Dystrophy |
| | TG | | | (DMD) |
| 1087 | GUGAaauguUuaggaCCACU | 4-5-1-5-7 | Vascular endothelial growth | Ulcers associated with type |
| | AG | | factor-A (VEGF-A) | II diabetes |
| 1091 | ACAUcaucaGaccagGCACC | 4-5-1-5-10 | COX2 | Inflammation and injury |
| | AGACC | | | |
| 1093 | AGAAguuggCaugguAGCCC | 4-5-1-5-10 | TGF-β1/COX-2 | Keloid scarless healing |
| | UUGGG | | | |
| 1094 | CTCCaacatCaaggaAGATG | 4-5-1-5-13 | Exon 51 Dystrophin | Duchenne muscular dystrophy |
| | GCATTTCT | | | |
| 1095 | GTTGcctccGgttctGAAGG | 4-5-1-5-10 | Exon 53 Dystrophin | Duchenne muscular dystrophy |
| | TGTTC | | | |
| 1097 | CGGAaucagUgaaugCUUAU | 4-5-1-4-13 | VEGF protein | Age-related macular |
| | ACAUCCGT | | | degeneration |
| 1099 | AUCAuaggaAacaccAAAGA | 4-5-1-4-13 | Cystic fibrosis transmembrane | Cystic fibrosis |
| | UGAUAUUU | | conductance regulator | |
| | | | (ΔF508 mutation) | |
| 1101 | UAGCaccauUugaaaUCAGU | 4-5-1-5-10 | miR-29 | Keloid scarless healing |
| | GUUUU | | | |
| 1105 | TATCcggagGgctcgCCATG | 4-5-1-5-10 | INSR inhibitor | keratitis-related progressive |
| | CTGCT | | | corneal neovascularization |
| 1106 | GTAAttgcgGcaagaAGAAT | 4-5-1-5-13 | CJA1 | Varicose Ulcer |
| | TGTTTCTG | | | |
| 1107 | TTGTgtcttTcttctGTTAG | 4-5-1-5-9 | Exon44 | Duchenne muscular dystrophy |
| | CCAC | | | |

*In Table 1C, lowercase font represents a 2′-deoxynucleoside, and uppercase font represents a 2′-O-methoxyethyl nucleoside, 2′-O-methoxyethyl cytidine, 2′-deoxy cytidine, 2′-O-methyl nucleoside, 2′-deoxy cytidine, 2′-O-methyl nucleoside, 2′-OH nucleoside, GNA, LNA, LNA with cytosine base, 2′-Fluoro 2′-deoxy nucleoside, 2′-Fluoro 2′-deoxy cytidine, 2′-F-Arabinonucleic Acid (2′-F-ANA), or 2′-F-Arabinonucleic Acid (2′-F-ANA) as described herein, or a combination thereof.
†Motif shows the regions of each sequence from 5′ to 3′ (e.g., W1-G1-W2 or W1-G1-S1-G2-W2), where the separator (S1) position is shown in bolded font.

TABLE 11A
Melting temperatures of oligonucleotides with or without separator sequences

| | Oligonucleotides with | | | | Oligonucleotides without | | | |
|---|---|---|---|---|---|---|---|---|
| | separator sequence | | | | separator sequence | | | |
| | | | SEQ | | | | SEQ | |
| | | | ID | Tm | | | ID | Tm |
| Target | Sequence | Motif* | NO: | (° C.) | Sequence | Motif* | NO: | (° C.) |
| APOCIII | AGCUucttgTccagcUUUAU | 4-5-1-5-5 | 701 | 61.00 | AGCUUcttgtccagcUUUAU | 5-10-5 | 772 | 64.00 |
| IE2 mRNA | GCGTttgctCttcttCTTGC | 4-5-1-5-5 | 702 | 62.50 | GCGTTtgctcttcttCTTGC | 5-10-5 | 773 | 65.00 |
| ApoB mRNA | GCCUcagtcTgcttcGCACC | 4-5-1-5-5 | 703 | 68.50 | GCCUCagtctgcttcGCACC | 5-10-5 | 774 | 71.50 |
| VEGF protein | CGGAaucagUgaaugCUUAU | 4-5-1-5-5 | 704 | 59.00 | CGGAAucagugaaugCUUAU | 5-10-5 | 775 | 61.00 |
| Exon 51 | CTCCaacatCaaggaAGATG | 4-5-1-5-5 | 705 | 60.50 | CTCCAacatcaaggaAGATG | 5-10-5 | 776 | 63.50 |
| Dystrophin | | | | | | | | |
| SMN2 mRNA | UCACuuucaUaaugcUGG | 4-5-1-5-3 | 706 | 53.00 | UCACuuucauaaugcUGG | 4-11-3 | 777 | 55.00 |
| Transthyretin | UCUUggttaCatgaaAUCCC | 4-5-1-5-5 | 707 | 57.50 | UCUUGgttacatgaaAUCCC | 5-10-5 | 778 | 61.50 |
| mRNA | | | | | | | | |
| Transthyretin | AUGGaauacUcuuggUUACT | 4-5-1-5-5 | 708 | 55.50 | AUGGAauacucuuggUUACT | 5-10-5 | 779 | 59.00 |
| mRNA | | | | | | | | |
| ALAS1 mRNA | CAGAaagagUgucucAUCUU | 4-5-1-5-5 | 709 | 58.00 | CAGAAagagugucucAUCUU | 5-10-5 | 780 | 58.50 |
| Exon 53 | GTTGcctccGgttctGAAGG | 4-5-1-5-5 | 710 | 69.00 | GTTGCctccggttctGAAGG | 5-10-5 | 781 | 70.50 |
| Dystrophin | | | | | | | | |
| Exon 53 | CCTCcggttCtgaagGTGTT | 4-5-1-5-5 | 711 | 65.50 | CCTCCggttctgaagGTGTT | 5-10-5 | 782 | 69.50 |
| Dystrophin | | | | | | | | |
| Exon 45 | CAATgccatCctggaGTTCC | 4-5-1-5-5 | 712 | 65.50 | CAATGccatcctggaGTTCC | 5-10-5 | 783 | 68.50 |
| Dystrophin | | | | | | | | |
| ICAM-1 | GCCCaagctGgcatcCGTCA | 4-5-1-5-5 | 713 | 72.50 | GCCCAagctggcatcCGTCA | 5-10-5 | 784 | 75.50 |
| inhibitor | | | | | | | | |
| INSR inhibitor | TATCcggagGgctcgCCATG | 4-5-1-5-5 | 714 | 72.00 | TATCCggagggctcgCCATG | 5-10-5 | 785 | 72.50 |
| Apo(a) | UGCUccgttGgtgctUGUUC | 4-5-1-5-5 | 715 | 67.50 | UGCUCcgttggtgctUGUUC | 5-10-5 | 786 | 69.00 |
| TTR | UCUUggttaCatgaaAUCCC | 4-5-1-5-5 | 716 | 57.50 | UCUUGgttacatgaaAUCCC | 5-10-5 | 787 | 61.00 |
| GFAP | CAGUautacCtctacTAGUC | 4-5-1-5-5 | 717 | 58.50 | CAGUAutacctctacTAGUC | 5-10-5 | 788 | 61.00 |
| FUS | GCAAugtcaCctttcAUACC | 4-5-1-5-5 | 718 | 59.00 | GCAAUgtcacctttcAUACC | 5-10-5 | 789 | 62.50 |
| SOD1 | CAGGatacaTttctaCAGCU | 4-5-1-5-5 | 719 | 60.50 | CAGGAtacatttctaCAGCU | 5-10-5 | 790 | 63.00 |
| HTT | CUCAgtaacAttgacACCAC | 4-5-1-5-5 | 720 | 61.00 | CUCAGtaacattgacACCAC | 5-10-5 | 791 | 64.00 |
| Transient | UGAAguagaAgaugcGCUU | 4-5-1-5-4 | 721 | 59.50 | UGAAguagaagaugcGCUU | 4-11-4 | 792 | 61.50 |
| receptor | | | | | | | | |
| potential cation | | | | | | | | |
| channel | | | | | | | | |
| subfamily V | | | | | | | | |
| Member 1 | | | | | | | | |
| Antithrombin | GGUUaacacCauuuaCUUCA | 4-5-1-5-5 | 722 | 54.50 | GGUUAacaccauuuaCUUCA | 5-10-5 | 793 | 57.00 |
| TGF-β2 | CGGCatgtcTattttGTA | 4-5-1-5-3 | 723 | 56.00 | CGGCatgtctattttGTA | 4-11-3 | 794 | 58.00 |
| inhibitor | | | | | | | | |
| CJA1 | GTAAttgcgGcaagaAGAAT | 4-5-1-5-5 | 724 | 60.00 | GTAATtgcggcaagaAGAAT | 5-10-5 | 795 | 62.50 |
| Centrosomal | GGUggaucAcgaguUCA | 3-5-1-5-3 | 725 | 59.0 | GGUggaucacgaguUCA | 3-11-3 | 796 | 62.5 |
| protein 290 | | | | | | | | |
| APOCIII | AGCUucttgTccagcUUUAU | 4-5-1-5-5 | 726 | 60.5 | AGCUUcttgtccagcUUUAU | 5-10-5 | 797 | 63.5 |
| Proprotein | AAGAucuggAcaaaaCGAAA | 4-5-1-5-5 | 727 | 58.5 | AAGAUcuggacaaaaCGAAA | 5-10-5 | 798 | 62.0 |
| convertase | | | | | | | | |
| subtilisin/kexin | | | | | | | | |
| type 12 | | | | | | | | |
| Angiotensinogen | CACAaacaaGctggtCGGTT | 4-5-1-5-5 | 728 | 66.0 | CACAAacaagctggtCGGTT | 5-10-5 | 799 | 67.5 |
| SERPINA1 gene/ | UUUAagaagAcaaagGGUUU | 4-5-1-5-5 | 729 | 56.5 | UUUAAgaagacaaagGGUUU | 5-10-5 | 800 | 59.0 |
| alpha-1 | | | | | | | | |
| antitrypsin | | | | | | | | |
| Mutant of α1- | UGUUaaacaUgccuaAACGC | 4-5-1-5-5 | 730 | 62.0 | UGUUAaacaugccuaAACGC | 5-10-5 | 801 | 63.5 |
| antitrypsin (Z- | | | | | | | | |
| AAT) | | | | | | | | |
| DGAT2 | TGCCatttaAtgagcTTCAC | 4-5-1-5-5 | 731 | 58.0 | TGCCAtttaatgagcTTCAC | 5-10-5 | 802 | 60.0 |
| STAT3 | CUAtttggAtgtcaGC | 3-5-1-5-2 | 732 | 51.5 | CUAtttggatgtcaGC | 3-11-2 | 803 | 54.5 |
| FOXP3 | GATtttgaCattctGC | 3-5-1-5-2 | 733 | 48.5 | GATtttgacattctGC | 3-11-2 | 804 | 50.5 |
| miR-155 | CACgauuAgcauUA | 3-4-1-4-2 | 734 | 46.0 | CACgauuagcauUA | 3-9-2 | 805 | 49.5 |
| Hepatitis B | UUAUugugaGgauuuUUGUC | 4-5-1-5-5 | 735 | 53.0 | UUAUUgugaggauuuUUGUC | 5-10-5 | 806 | 54.0 |
| surface antigen | | | | | | | | |
| Hepatitis B | GCAGaggtgAagcgaAGUGC | 4-5-1-5-5 | 736 | 69.0 | GCAGAggtgaagcgaAGUGC | 5-10-5 | 807 | 71.5 |
| viral | | | | | | | | |
| proteins | | | | | | | | |
| (including | | | | | | | | |
| surface | | | | | | | | |
| antigen) | | | | | | | | |
| Hepatitis B | UGUGaagcgAagugcACACU | 4-5-1-5-5 | 737 | 64.5 | UGUGAagcgaagugcACACU | 5-10-5 | 808 | 67.0 |
| surface antigen | | | | | | | | |
| ATXN2 | CTTCacattTcgatcCAACA | 4-5-1-5-5 | 738 | 59.0 | CTTCAcatttcgatcCAACA | 5-10-5 | 809 | 61.5 |
| DNM2 | GGCataagGtcacgGA | 3-5-1-5-2 | 739 | 63.0 | GGCataaggtcacgGA | 3-11-2 | 810 | 64.0 |
| Exon 51 | CTCCaacatCaaggaAGATG | 4-5-1-5-5 | 740 | 61.0 | CTCCAacatcaaggaAGATG | 5-10-5 | 811 | 63.5 |
| Dystrophin | | | | | | | | |
| Caspase 2 | AGGAguuccAcauucUGGC | 4-5-1-5-4 | 741 | 64.5 | AGGAguuccacauucUGGC | 4-11-4 | 812 | 68.0 |
| RTP801 | AGCUgcaucAgguugGCAC | 4-5-1-5-4 | 742 | 67.0 | AGCUgcaucagguugGCAC | 4-11-4 | 813 | 69.0 |
| β2-Adrenergic | CUGGaucacAugcacAAUGT | 4-5-1-5-5 | 743 | 65.0 | CUGGAucacaugcacAAUGT | 5-10-5 | 814 | 67.5 |
| receptor | | | | | | | | |
| miR-21 | ACATcagtcTgauaaGCTA | 4-5-1-5-4 | 744 | 56.5 | ACATcagtctgauaaGCTA | 4-11-4 | 815 | 59.5 |
| Complement | AUCCcacgcCcctgtCCAGC | 4-5-1-5-5 | 745 | 75.5 | AUCCCacgcccctgtCCAGC | 5-10-5 | 816 | 78.5 |
| Factor B | | | | | | | | |
| Complement | AGCAagauaUuuuuaUAAUA | 4-5-1-5-5 | 746 | 41.5 | AGCAAgauauuuuuaUAAUA | 5-10-5 | 817 | 45.5 |
| component C5 | | | | | | | | |
| TMPRSS6 | GCTTagagtAcagccCACTT | 4-5-1-5-5 | 747 | 63.5 | GCTTAgagtacagccCACTT | 5-10-5 | 818 | 66.5 |
| Factor XI | ACGGcattgGtgcacAGUUU | 4-5-1-5-5 | 748 | 68.5 | ACGGCattggtgcacAGUUU | 5-10-5 | 819 | 71.0 |
| miR-29 | UAGCaccauUugaaaUCAGU | 4-5-1-5-5 | 749 | 58.5 | UAGCAccauuugaaaUCAGU | 5-10-5 | 820 | 61.0 |
| TGF-β1/COX-2 | AGAAguuggCaugguAGCCC | 4-5-1-5-5 | 750 | 66.0 | AGAAGuuggcaugguAGCCC | 5-10-5 | 821 | 68.5 |
| Vascular | GUGAaauguUuaggaCCACU | 4-5-1-5-5 | 751 | 57.5 | GUGAAauguuuaggaCCACU | 5-10-5 | 822 | 60.0 |
| endothelial | | | | | | | | |
| growth factor-A | | | | | | | | |
| (VEGF-A) | | | | | | | | |
| GHr | CCACctttgGgtgaaTAGCA | 4-5-1-5-5 | 752 | 63.0 | CCACCtttgggtgaaTAGCA | 5-10-5 | 823 | 65.0 |
| PKK | TGCAagtctCttggcAAACA | 4-5-1-5-5 | 753 | 63.5 | TGCAAgtctcttggcAAACA | 5-10-5 | 824 | 66.5 |
| CD49d | CTGAgtctgTtttccATTCT | 4-5-1-5-5 | 754 | 58.5 | CTGAGtctgttttccATTCT | 5-10-5 | 825 | 61.0 |
| antagonists | | | | | | | | |
| TGF-β2 | GAccagAtgcagGA | 2-4-1-5-2 | 755 | 58.0 | GAccagatgcagGA | 2-10-2 | 826 | 61.5 |
| inhibitor | | | | | | | | |
| Exon44 | TTGTgtcttTcttctGTTAG | 4-5-1-5-5 | 756 | 55.0 | TTGTGtctttcttctGTTAG | 5-10-5 | 827 | 59.0 |
| Nav1.1 | AGUUggagcAagauuAUC | 4-5-1-5-3 | 757 | 53.5 | AGUUggagcaagauuAUC | 4-11-3 | 828 | 55.5 |
| Protein kinase | UUGUccaggAaguccUCAAG | 4-5-1-5-5 | 758 | 64.5 | UUGUCcaggaaguccUCAAG | 5-10-5 | 829 | 65.5 |
| N3 | | | | | | | | |
| Cystic fibrosis | AUCAuaggaAacaccAAAGA | 4-5-1-5-5 | 759 | 60.5 | AUCAUaggaaacaccAAAGA | 5-10-5 | 830 | 62.0 |
| transmembrane | | | | | | | | |
| conductance | | | | | | | | |
| regulator | | | | | | | | |
| (ΔF508 | | | | | | | | |
| mutation) | | | | | | | | |
| Hepatitis B | UACCaauuuAugccuACAGC | 4-5-1-5-5 | 760 | 57.5 | UACCAauuuaugccuACAGC | 5-10-5 | 831 | 61.0 |
| surface antigen | | | | | | | | |
| Mutant of α-1 | AAACccuuuGucuucUUAA | 4-5-1-5-4 | 761 | 55.5 | AAACccuuugucuucUUAA | 4-11-4 | 832 | 58.0 |
| antitrypsin | | | | | | | | |
| TAU | CCGTtttctTaccacCCT | 4-5-1-5-3 | 762 | 65.0 | CCGTtttcttaccacCCT | 4-11-3 | 833 | 67.0 |
| DMPK | UUCGaagacAguucuAGGGU | 4-5-1-5-5 | 763 | 63.0 | UUCGAagacaguucuAGGGU | 5-10-5 | 834 | 64.5 |
| Usherin (exon | AGCUucggaGaaauuUAAAU | 4-5-1-5-5 | 764 | 54.0 | AGCUUcggagaaauuUAAAU | 5-10-5 | 835 | 55.5 |
| 13 of USH2A) | | | | | | | | |
| PCSK9 | GGGCtcataGcacatTATCC | 4-5-1-5-5 | 765 | 64.00 | GGGCTcatagcacatTATCC | 5-10-5 | 836 | 67.00 |
| DMPK | ACAGacaatAaatacCGAGG | 4-5-1-5-5 | 766 | 59.00 | ACAGAcaataaatacCGAGG | 5-10-5 | 837 | 60.00 |
| TTR | TACAaatggGatgctACTGC | 4-5-1-5-5 | 767 | 60.50 | TACAAatgggatgctACTGC | 5-10-5 | 838 | 61.50 |
| AAT | ACCCaattcAgaaggAAGGA | 4-5-1-5-5 | 768 | 62.00 | ACCCAattcagaaggAAGGA | 5-10-5 | 839 | 64.50 |
| DMPK | TCGAagacaGttctaGGGTT | 4-5-1-5-5 | 769 | 63.00 | TCGAAgacagttctaGGGTT | 5-10-5 | 840 | 65.00 |
| DMPK | CCCAgcgccCaccagTCACA | 4-5-1-5-5 | 770 | 76.50 | CCCAGcgcccaccagTCACA | 5-10-5 | 841 | 79.50 |
| DMPK | CTTCccgaaTgtccgACAGT | 4-5-1-5-5 | 771 | 68.50 | CTTCCcgaatgtccgACAGT | 5-10-5 | 842 | 68.00 |

*Motifs show the regions of each sequence from 5′ to 3′ (e.g., W1-G1-W2 or W1-G1-S1-G2-W2), where the separator sequence is shown in a bolded font.

### Enumerated Embodiments

The following sets of enumerated embodiments are provided as exemplary.

### Set I

Embodiment I-1. A compound comprising a modified oligonucleotide, the modified oligonucleotide comprising from 5′ to 3′:

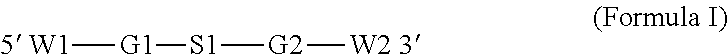

wherein:
W1 is a 5′ wing segment;
W2 is a 3′ wing segment;
G1 is a first gap segment;
S1 is a first separator segment;
G2 is a second gap segment;
- is an internucleoside linkage; and
at least one nucleoside of the oligonucleotide is modified.

Embodiment I-2. The compound of embodiment I-1, wherein the modified oligonucleotide comprises from 5′ to 3′:

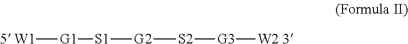

wherein:
S2 is a second separator segment; and
G3 is a third gap segment.

Embodiment I-3. The compound of embodiment I-2, wherein the modified oligonucleotide comprises from 5′ to 3′:

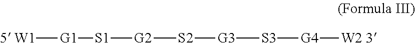

wherein:
S3 is a third separator segment; and
G4 is a fourth gap segment.

Embodiment I-4. The compound of embodiment I-3, wherein the modified oligonucleotide comprises from 5′ to 3′:

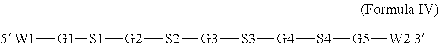

wherein:
S4 is a fourth separator segment; and
G5 is a fifth gap segment.

Embodiment I-5. The compound of embodiment I-4, wherein the modified oligonucleotide comprises from 5′ to 3′:

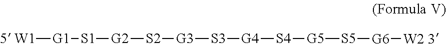

wherein:
S5 is a fifth separator segment; and
G6 is a sixth gap segment.

Embodiment I-6. The compound of embodiment I-5, wherein the modified oligonucleotide comprises from 5′ to 3′:

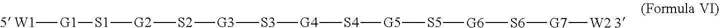

wherein:
S6 is a sixth separator segment; and
G7 is a seventh gap segment.

Embodiment I-7. The compound of any one of embodiments I-1 to I-6, wherein W1 comprises one or more linked deoxynucleosides.

Embodiment I-8. The compound of any one of embodiments I-1 to I-7, wherein W2 comprises one or more linked deoxynucleosides.

Embodiment I-9. The compound of any one of embodiments I-1 to I-8, wherein W1 comprises 2 to 25 linked nucleosides.

Embodiment I-10. The compound of any one of embodiments I-1 to I-9, wherein W2 comprises 2 to 35 linked nucleosides.

Embodiment I-11. The compound of any one of embodiments I-1 to I-10, wherein G1 comprises 1 to 10 linked deoxynucleosides.

Embodiment I-12. The compound of any one of embodiments I-1 to I-11, wherein G2 comprises 1 to 10 linked deoxynucleosides.

Embodiment I-13. The compound of any one of embodiments I-1 to I-12, wherein S1 comprises 1 to 3 linked nucleosides.

Embodiment I-14. The compound of any one of embodiments I-2 to I-13, wherein G3 comprises 1 to 10 linked deoxynucleosides.

Embodiment I-15. The compound of any one of embodiments I-2 to I-14, wherein S2 comprises 1 to 3 linked nucleosides.

Embodiment I-16. The compound of any one of embodiments I-3 to I-15, wherein G4 comprises 1 to 10 linked deoxynucleosides.

Embodiment I-17. The compound of any one of embodiments I-3 to I-16, wherein S3 comprises 1 or 2 linked nucleosides.

Embodiment I-18. The compound of any one of embodiments I-4 to I-17, wherein G5 comprises 1 to 10 linked deoxynucleosides.

Embodiment I-19. The compound of any one of embodiments I-4 to I-17, wherein S4 comprises 1 to 3 linked nucleosides.

Embodiment I-20. The compound of any one of embodiments I-5 to I-19, wherein G6 comprises 1 to 10 linked deoxynucleosides.

Embodiment I-21. The compound of any one of embodiments I-5 to I-19, wherein S5 comprises 1 to 3 linked nucleosides.

Embodiment I-22. The compound of any one of embodiments I-6 to I-21, wherein G7 comprises 1 to 10 linked deoxynucleosides.

Embodiment I-23. The compound of any one of embodiments I-6 to I-22, wherein S6 comprises 1 to 3 linked nucleosides.

Embodiment I-24. The compound of any one of embodiments I-1 to I-23, wherein the oligonucleotide is 18-50 nucleobases in length.

Embodiment I-25. The compound of any one of embodiments I-1 to I-23, wherein the oligonucleotide is at least 20 nucleobases in length.

Embodiment I-26. The compound of any one of embodiments I-1 to I-23, wherein the oligonucleotide is 20 nucleobases in length.

Embodiment I-27. The compound of embodiment I-1, wherein the oligonucleotide is 20 linked nucleobases in length.

Embodiment I-28. The compound of embodiment I-27, wherein W1 comprises 4 linked nucleosides.

Embodiment I-29. The compound of embodiment I-27, wherein W1 comprises 5 linked nucleosides.

Embodiment I-30. The compound of any one of embodiments I-27 to I-29, wherein G1 comprises 4 linked deoxynucleosides.

Embodiment I-31. The compound of any one of embodiments I-27 to I-29, wherein G1 comprises 5 linked deoxynucleosides.

Embodiment I-32. The compound of any one of embodiments I-27 to I-29, wherein G1 comprises 6 linked deoxynucleosides.

Embodiment I-33. The compound of any one of embodiments I-27 to I-32, wherein G2 comprises 4 linked deoxynucleosides.

Embodiment I-34. The compound of any one of embodiments I-27 to I-32, wherein G2 comprises 5 linked deoxynucleosides.

Embodiment I-35. The compound of any one of embodiments I-27 to I-32, wherein G2 comprises 6 linked deoxynucleosides.

Embodiment I-36. The compound of any one of embodiments I-27 to I-33, wherein S1 comprises at least one linked nucleoside.

Embodiment I-37. The compound of any one of embodiments I-27 to I-35, wherein W2 comprises 5 linked nucleosides.

Embodiment I-38. The compound of any one of embodiments I-27 to I-35, wherein W2 comprises 6 linked nucleosides.

Embodiment I-39. The compound of any one of embodiments I-27 to I-38, wherein the S1 is at position 10 of the oligonucleotide.

Embodiment I-40. The compound of embodiment I-39, wherein W1 comprises 4 linked nucleosides, G1 comprises 5 linked deoxynucleosides, S1 comprises 1 linked nucleoside, G2 comprises 5 linked deoxynucleosides, and W2 comprises 5 linked nucleosides.

Embodiment I-41. The compound of embodiment I-1, wherein W1 comprises 4-6 linked nucleosides.

Embodiment I-42. The compound of embodiment I-41, wherein W2 comprises 4-6 linked nucleosides.

Embodiment I-43. The compound of any one of embodiments I-41 to I-42, wherein G1 comprises 1-6 linked deoxynucleosides.

Embodiment I-44. The compound of any one of embodiments I-41 to I-43, wherein G2 comprises 1-6 linked deoxynucleosides.

Embodiment I-45. The compound of any one of embodiments I-41 to I-44, wherein G3 comprises 1-6 linked deoxynucleosides.

Embodiment I-46. The compound of any one of embodiments I-41 to I-45, wherein S1 comprises least one linked nucleoside.

Embodiment I-47. The compound of any one of embodiments I-41 to I-46, wherein the length of the G1-S1-G2 is between 8 and 12 nucleobases in length.

Embodiment I-48. The compound of any one of embodiments I-1 to I-47, wherein any one or more of G1, G2, G3, G4, G5, G6, and G7 comprises a nucleoside comprising a modification to a 2′-deoxynucleoside.

Embodiment I-49. The compound of embodiment I-48, wherein any one or more of G1, G2, G3, G4, G5, G6, and G7 comprises a nucleoside comprising a 2′-deoxy 5-methylcytidine sugar modification.

Embodiment I-50. The compound of any one of embodiments I-1 to I-49, wherein S1, S2, S3, S4, S5, and/or S6 comprises a nucleoside comprising a 2′-O-methoxyethyl sugar modification.

Embodiment I-51. The compound of any one of embodiments I-1 to I-49, wherein S1, S2, S3, S4, S5, and/or S6 comprises a nucleoside comprising a 5-methylcytidine.

Embodiment I-52. The compound of any one of embodiments I-1 to I-49, wherein S1, S2, S3, S4, S5, and/or S6 comprises a nucleoside comprising a 2′-O-methyl sugar modification.

Embodiment I-53. The compound of any one of embodiments I-1 to I-52, wherein S1, S2, S3, S4, S5, and/or S6 comprises a nucleoside comprising a 2′-OH sugar modification.

Embodiment I-54. The compound of any one of embodiments I-1 to I-53, wherein S1, S2, S3, S4, S5, and/or S6 comprises a nucleoside comprising a 2′-fluoro sugar modification.

Embodiment I-55. The compound of any one of embodiments I-1 to I-54, wherein S1, S2, S3, S4, S5, and/or S6 comprises a nucleoside comprising a 2′-fluoro-arabinonucleic acid (2′-fluoro-ANA) sugar modification.

Embodiment I-56. The compound of any one of embodiments I-1 to I-55, wherein S1, S2, S3, S4, S5, and/or S6 comprises a glycol nucleic acid (GNA).

Embodiment I-57. The compound of any one of embodiments I-1 to I-56, wherein S1, S2, S3, S4, S5, and/or S6 comprises a LNA.

Embodiment I-58. The compound of any one of embodiments I-1 to I-57, wherein W1 comprises a nucleoside comprising a 2′-deoxy sugar modification.

Embodiment I-59. The compound of any one of embodiments I-1 to I-58, wherein W1 comprises a nucleoside comprising a 2′-O-methoxyethyl sugar modification.

Embodiment I-60. The compound of any one of embodiments I-1 to I-59, wherein W1 comprises a nucleoside comprising a 2′-O-methyl sugar modification.

Embodiment I-61. The compound of any one of embodiments I-1 to I-60, wherein W1 comprises a nucleoside comprising a 2′-fluoro sugar modification.

Embodiment I-62. The compound of any one of embodiments I-1 to I-61, wherein W1 comprises a nucleoside comprising a 2′-fluoro-arabinonucleic acid (2′-fluoro-ANA) modification.

Embodiment I-63. The compound of any one of embodiments I-1 to I-62, wherein W1 comprises a glycol nucleic acid (GNA).

Embodiment I-64. The compound of any one of embodiments I-1 to I-63, wherein W1 comprises a modified nucleoside and wherein the modified nucleoside is a locked nucleic acid (LNA).

Embodiment I-65. The compound of embodiment I-64, wherein the LNA is at positions 1 and/or 3 of a sequence corresponding to SEQ ID NO: 2.

Embodiment I-66. The compound of any one of embodiments I-1 to I-65, wherein W2 comprises a nucleoside comprising a 2′-deoxy sugar modification.

Embodiment I-67. The compound of any one of embodiments I-1 to I-66, wherein W2 comprises a nucleoside comprising a 2′-O-methoxyethyl sugar modification.

Embodiment I-68. The compound of any one of embodiments I-1 to I-67, wherein W2 comprises a nucleoside comprising a 2′-O-methyl sugar modification.

Embodiment I-69. The compound of any one of embodiments I-1 to I-68, wherein W2 comprises a nucleoside comprising a 2′-fluoro sugar modification.

Embodiment I-70. The compound of any one of embodiments I-1 to I-69, wherein W2 comprises a nucleoside comprising a 2′-fluoro-arabinonucleic acid (2′-fluoro-ANA) modification.

Embodiment I-71. The compound of any one of embodiments I-1 to I-70, wherein W2 comprises a modified nucleoside and wherein the modified nucleoside is a glycol nucleic acid (GNA).

Embodiment I-72. The compound of any one of embodiments I-1 to I-71, wherein W2 comprises a modified nucleoside and wherein the modified nucleoside is a locked nucleic acid (LNA).

Embodiment I-73. The compound of any one of embodiments I-1 to I-72, wherein at least one internucleoside linkage is a phosphodiester linkage.

Embodiment I-74. The compound of any one of embodiments I-1 to I-73, wherein at least one internucleoside linkage is a modified internucleoside linkage.

Embodiment I-75. The compound of embodiment I-74, wherein the modified internucleoside linkage is a phosphorothioate internucleoside linkage.

Embodiment I-76. The compound of any one of embodiments I-1 to I-75, wherein the target of the oligonucleotide is a viral target, optionally wherein the viral target is selected from a HPV, EBV, HSV, HIV, or RSV target.

Embodiment I-77. The compound of any one of embodiments I-1 to I-75, wherein the target of the oligonucleotide is a not HBV.

Embodiment I-78. The compound of any one of embodiments I-1 to I-75, wherein the target of the oligonucleotide is a human target.

Embodiment I-79. The compound of any one of embodiments I-1 to I-75, wherein the target of the oligonucleotide is an oncogenic target, an inflammatory target, a metabolic target, a cardiovascular target, a neurological target, a neuropsychiatric target, or the like.

Embodiment I-80. A compound comprising the structure of the modified oligonucleotide of any one of SEQ ID NOS: 11-666.

Embodiment I-81. A pharmaceutical composition comprising the compound of any one of embodiments I-1 to I-80, or salt thereof and at least one pharmaceutically acceptable carrier or diluent.

Embodiment I-82. A method of treating a disease, disorder, or condition comprising administering to a subject the compound of any one of embodiments I-1 to I-80, or the composition of embodiment I-81.

Embodiment I-83. The method of embodiment I-82, wherein the administering to a subject is parenteral.

Embodiment I-84. The method of embodiment I-82, wherein the administering to a subject is subcutaneous.

Embodiment I-85. The method of embodiment I-82, wherein the administering to a subject is transdermal.

Embodiment I-86. The method of embodiment I-82, wherein the administering to a subject is intraocular.

Embodiment I-87. The method of embodiment I-82, wherein the administering to a subject is intramuscular.

Embodiment I-88. The method of embodiment I-82, wherein the administering to a subject is intravenous.

Embodiment I-89. The method of any one of embodiments I-82 to I-88, wherein the subject is human.

### Set II

Embodiment II-1. A modified multi-segmented antisense oligonucleotide (“modified oligonucleotide”) that is complementary to a nucleotide sequence in a gene transcript, wherein the gene is DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) or hydroxy acid oxidase 1,
wherein the modified oligonucleotide comprises from 5′ to 3′ order:

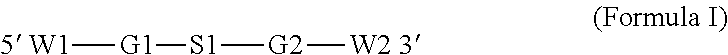

wherein:
W1 is a 5′ wing segment;
W2 is a 3′ wing segment;
G1 is a first gap segment;
S1 is a first separator segment;
G2 is a second gap segment,
wherein each segment is joined by an internucleoside linkage, and
wherein at least one nucleoside of the modified oligonucleotide is a modified nucleoside.

Embodiment II-2. The modified oligonucleotide of embodiment II-1, wherein the modified oligonucleotide comprises from 5′ to 3′ order:

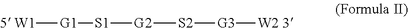

wherein:
S2 is a second separator segment; and
G3 is a third gap segment.

Embodiment II-3. The modified oligonucleotide of any one of embodiments II-1 to II-2, wherein W1 comprises one or more linked deoxynucleosides.

Embodiment II-4. The modified oligonucleotide of any one of embodiments II-1 to II-3, wherein W2 comprises one or more linked deoxynucleosides.

Embodiment II-5. The modified oligonucleotide of any one of embodiments II-1 to II-4, wherein W1 comprises 2 to 25 linked nucleosides.

Embodiment II-6. The modified oligonucleotide of any one of embodiments II-1 to II-5, wherein W2 comprises 2 to 35 linked nucleosides.

Embodiment II-7. The modified oligonucleotide of any one of embodiments II-1 to II-6, wherein G1 comprises 1 to 10 linked deoxynucleosides.

Embodiment II-8. The modified oligonucleotide of any one of embodiments II-1 to II-7, wherein G2 comprises 1 to 10 linked deoxynucleosides.

Embodiment II-9. The modified oligonucleotide of any one of embodiments II-1 to II-8, wherein S1 comprises 1 to 3 linked nucleosides.

Embodiment II-10. The modified oligonucleotide of any one of embodiments II-2 to II-9, wherein G3 comprises 1 to 10 linked deoxynucleosides.

Embodiment II-11. The modified oligonucleotide of any one of embodiments II-2 to II-10, wherein S2 comprises 1 to 3 linked nucleosides.

Embodiment II-12. The modified oligonucleotide of any one of embodiments II-1 to II-11, wherein the modified oligonucleotide is 18-50 nucleobases in length.

Embodiment II-13. The modified oligonucleotide of any one of embodiments II-1 to II-12, wherein the modified oligonucleotide is at least 20 nucleobases in length.

Embodiment II-14. The modified oligonucleotide of any one of embodiments II-1 to II-12, wherein the modified oligonucleotide is 20 nucleobases in length.

Embodiment II-15. The modified oligonucleotide of embodiment II-1, wherein the modified oligonucleotide is 20 linked nucleosides in length.

Embodiment II-16. The modified oligonucleotide of embodiment II-15, wherein W1 comprises 4 linked nucleosides.

Embodiment II-17. The modified oligonucleotide of embodiment II-15, wherein W1 comprises 5 linked nucleosides.

Embodiment II-18. The modified oligonucleotide of any one of embodiments II-15 to II-17, wherein G1 comprises 4 linked deoxynucleosides.

Embodiment II-19. The modified oligonucleotide of any one of embodiments II-15 to II-17, wherein G1 comprises 5 linked deoxynucleosides.

Embodiment II-20. The modified oligonucleotide of any one of embodiments 11-15 to II-17, wherein G1 comprises 6 linked deoxynucleosides.

Embodiment II-21. The modified oligonucleotide of any one of embodiments II-15 to II-20, wherein G2 comprises 4 linked deoxynucleosides.

Embodiment II-22. The modified oligonucleotide of any one of embodiments 11-15 to II-20, wherein G2 comprises 5 linked deoxynucleosides.

Embodiment II-23. The modified oligonucleotide of any one of embodiments II-15 to II-20, wherein G2 comprises 6 linked deoxynucleosides.

Embodiment II-24. The modified oligonucleotide of any one of embodiments 11-15 to II-23, wherein S1 comprises at least one linked nucleoside.

Embodiment II-25. The modified oligonucleotide of any one of embodiments II-15 to II-24, wherein W2 comprises 5 linked nucleosides.

Embodiment II-26. The modified oligonucleotide of any one of embodiments 11-15 to II-23, wherein W2 comprises 6 linked nucleosides.

Embodiment II-27. The modified oligonucleotide of any one of embodiments II-15 to II-26, wherein the S1 is at position 10 of the modified oligonucleotide.

Embodiment II-28. The modified oligonucleotide of any one of embodiments II-15 to II-26, wherein the S1 is at position 11 of the modified oligonucleotide.

Embodiment II-29. The modified oligonucleotide of embodiment II-27, wherein W1 comprises 4 linked nucleosides, G1 comprises 5 linked deoxynucleosides, S1 comprises 1 linked nucleoside, G2 comprises 5 linked deoxynucleosides, and W2 comprises 5 linked nucleosides.

Embodiment II-30. The modified oligonucleotide of embodiment II-27, wherein W1 comprises 5 linked nucleosides, G1 comprises 4 linked deoxynucleosides, S1 comprises 1 linked nucleoside, G2 comprises 5 linked deoxynucleosides, and W2 comprises 5 linked nucleosides.

Embodiment II-31. The modified oligonucleotide of embodiment II-27, wherein W1 comprises 5 linked nucleosides, G1 comprises 5 linked deoxynucleosides, S1 comprises 1 linked nucleoside, G2 comprises 4 linked deoxynucleosides, and W2 comprises 5 linked nucleosides.

Embodiment II-32. The modified oligonucleotide of embodiment II-28, wherein W1 comprises 4 linked nucleosides, G1 comprises 6 linked deoxynucleosides, S1 comprises 1 linked nucleoside, G2 comprises 4 linked deoxynucleosides, and W2 comprises 5 linked nucleosides.

Embodiment II-33. The modified oligonucleotide of any one of embodiments II-1 to II-2, wherein W1 comprises 4-6 linked nucleosides.

Embodiment II-34. The modified oligonucleotide of embodiment II-33, wherein W2 comprises 4-6 linked nucleosides.

Embodiment II-35. The modified oligonucleotide of any one of embodiments II-33 to II-34, wherein G1 comprises 1-6 linked deoxynucleosides.

Embodiment II-36. The modified oligonucleotide of any one of embodiments 11-33 to II-35, wherein G2 comprises 1-6 linked deoxynucleosides.

Embodiment II-37. The modified oligonucleotide of any one of embodiments II-33 to II-36, wherein G3 comprises 1-6 linked deoxynucleosides.

Embodiment II-38. The modified oligonucleotide of any one of embodiments 11-33 to II-37, wherein S1 comprises least one linked nucleoside.

Embodiment II-39. The modified oligonucleotide of any one of embodiments II-33 to II-38, wherein the total length of the G1, S1 and G2 is 8 to 12 nucleobases in length.

Embodiment II-40. The modified oligonucleotide of any one of embodiments 11-33 to II-38, wherein the total length of the G1, S1, G2, S2 and G3 is 8 to 12 nucleobases in length.

Embodiment II-41. The modified oligonucleotide of any one of embodiments II-1 to II-40, wherein any one or more of G1, G2, and G3, comprises a nucleoside comprising a modification to a 2′-deoxynucleoside.

Embodiment II-42. The modified oligonucleotide of embodiment II-41, wherein any one or more of G1, G2, G3, G4, G5, G6, and G7 comprises a nucleoside comprising a 2′-deoxy 5-methylcytidine sugar modification.

Embodiment II-43. The modified oligonucleotide of any one of embodiments II-1 to II-42, wherein S1 and/or S2 comprises a nucleoside comprising a 2′-O-methoxyethyl sugar modification.

Embodiment II-44. The modified oligonucleotide of any one of embodiments II-1 to II-43, wherein S1 and/or S2 comprises a nucleoside comprising a 5-methylcytidine.

Embodiment II-45. The modified oligonucleotide of any one of embodiments 11-1 to II-44, wherein S1 and/or S2 comprises a nucleoside comprising a 2′-O-methyl sugar modification.

Embodiment II-46. The modified oligonucleotide of any one of embodiments 11-1 to II-45, wherein S1 and/or S2 comprises a nucleoside comprising a 2′-OH sugar modification.

Embodiment II-47. The modified oligonucleotide of any one of embodiments II-1 to II-46, wherein S1 and/or S2 comprises a nucleoside comprising a 2′-fluoro sugar modification.

Embodiment II-48. The modified oligonucleotide of any one of embodiments II-1 to II-47, wherein S1 and/or S2 comprises a nucleoside comprising a 2′-fluoro-arabinonucleic acid (2′-fluoro-ANA) sugar modification.

Embodiment II-49. The modified oligonucleotide of any one of embodiments 11-1 to II-48, wherein S1 and/or S2 comprises a glycol nucleic acid (GNA).

Embodiment II-50. The modified oligonucleotide of any one of embodiments II-1 to II-49, wherein S1 and/or S2 comprises a locked nucleic acid (LNA).

Embodiment II-51. The modified oligonucleotide of any one of embodiments II-1 to II-50, wherein W1 comprises a nucleoside comprising a 2′-deoxy sugar modification.

Embodiment II-52. The modified oligonucleotide of any one of embodiments II-1 to II-51 wherein W1 comprises a 2′-O-methoxyethyl 5-methylcytidine.

Embodiment II-53. The modified oligonucleotide of any one of embodiments II-1 to II-52, wherein W1 comprises a nucleoside comprising a 2′-O-methyl sugar modification.

Embodiment II-54. The modified oligonucleotide of embodiment II-53, wherein W1 comprises a 2′-O-methyl 5-methylcytidine.

Embodiment II-55. The modified oligonucleotide of any one of embodiments II-1 to II-54, wherein W1 comprises a nucleoside comprising a 2′-fluoro sugar modification.

Embodiment II-56. The modified oligonucleotide of any one of embodiments II-1 to II-55, wherein W1 comprises a nucleoside comprising a 2′-fluoro-arabinonucleic acid (2′-fluoro-ANA) modification.

Embodiment II-57. The modified oligonucleotide of any one of embodiments II-1 to II-56, wherein W1 comprises a glycol nucleic acid (GNA).

Embodiment II-58. The modified oligonucleotide of any one of embodiments II-1 to II-57, wherein W1 comprises a modified nucleoside and wherein the modified nucleoside is a locked nucleic acid (LNA).

Embodiment II-59. The modified oligonucleotide of any one of embodiments II-1 to II-58, wherein W2 comprises a nucleoside comprising a 2′-deoxy sugar modification.

Embodiment II-60. The modified oligonucleotide of any one of embodiments II-1 to II-59, wherein W2 comprises a nucleoside comprising a 2′-O-methoxyethyl sugar modification.

Embodiment II-61. The modified oligonucleotide of embodiment II-60, wherein W2 comprises a 2′-O-methoxyethyl 5-methylcytidine.

Embodiment II-62. The modified oligonucleotide of any one of embodiments II-1 to II-61, wherein W2 comprises a nucleoside comprising a 2′-O-methyl sugar modification.

Embodiment II-63. The modified oligonucleotide of embodiment II-62, wherein W2 comprises a 2′-O-methyl 5-methylcytidine.

Embodiment II-64. The modified oligonucleotide of any one of embodiments II-1 to II-63, wherein W2 comprises a nucleoside comprising a 2′-fluoro sugar modification.

Embodiment II-65. The modified oligonucleotide of any one of embodiments II-1 to II-64, wherein W2 comprises a nucleoside comprising a 2′-fluoro-arabinonucleic acid (2′-fluoro-ANA) modification.

Embodiment II-66. The modified oligonucleotide of any one of embodiments IT-1 to IT-65, wherein W2 comprises a modified nucleoside and wherein the modified nucleoside is a glycol nucleic acid (GNA).

Embodiment II-67. The modified oligonucleotide of any one of embodiments IT-1 to IT-66, wherein W2 comprises a modified nucleoside and wherein the modified nucleoside is a locked nucleic acid (LNA).

Embodiment II-68. The modified oligonucleotide of any one of embodiments II-1 to II-67, wherein at least one internucleoside linkage is a phosphodiester linkage.

Embodiment II-69. The modified oligonucleotide of any one of embodiments II-1 to II-68, wherein at least one internucleoside linkage is a modified internucleoside linkage.

Embodiment II-70. The modified oligonucleotide of embodiment II-69, wherein the modified internucleoside linkage is a phosphorothioate internucleoside linkage.

Embodiment II-71. The modified oligonucleotide of any one of embodiments II-1 to II-70, wherein the oligonucleotide is at least 60%, 65%, 70%, 75%, 80%, 85%, 90%, 91%, 92%, 93%, 94%, 95%, 96%, 97%, 98%, 99% or 100% complementary to a portion of the polynucleotide sequence of any one of SEQ ID NOS: 369, 371, 373, 375, 377, 379, 381, 383, 385, 387, 389, 391, 393, 395, 397, 399, 401, 403, 405, 407, 409 and 411.

Embodiment II-72. The modified oligonucleotide of embodiment II-71, wherein the oligonucleotide is complementary to the polynucleotide sequence of any one of SEQ ID NOS: 370, 372, 374, 376, 378, 380, 382, 384, 386, 388, 390, 392, 394, 396, 398, 400, 402, 404, 408, 410 and 412.

Embodiment II-73. The modified oligonucleotide of any one of embodiments II-1 to II-72, wherein the oligonucleotide comprises a nucleobase sequence of SEQ ID NOS: 369, 371, 373, 375, 377, 379, 381, 383, 385, 387, 389, 391, 393, 395, 397, 399, 401, 403, 405, 407, 409 and 411.

Embodiment II-74. The modified oligonucleotide of any one of embodiments II-1 to II-70, wherein, modified oligonucleotide is complementary to PCSK9.

Embodiment II-75. The modified oligonucleotide of any one of embodiments II-1 to II-70, wherein, modified oligonucleotide is complementary to DMPK.

Embodiment II-76. The modified oligonucleotide of any one of embodiments II-1 to II-70, wherein, modified oligonucleotide is complementary to AAT.

Embodiment II-77. The modified oligonucleotide of any one of embodiments II-1 to II-70, wherein, modified oligonucleotide is complementary to TTR.

Embodiment II-78. A modified oligonucleotide comprising the modified oligonucleotide of any one of SEQ ID NOS: 1-368, or a oligonucleotide comprising at least 1, 2, 3, 4, or 5 further modifications thereto.

Embodiment II-79. A modified oligonucleotide comprising the modified oligonucleotide of any one of SEQ ID NOS: 1-368, or a oligonucleotide having at least 70%, at least 80%, at least 90%, at least 95%, or at least 99% sequence identity thereto.

Embodiment II-80. A pharmaceutical composition comprising the modified oligonucleotide of any one of embodiments II-1 to II-79, or salt thereof and at least one pharmaceutically acceptable carrier or diluent.

Embodiment II-81. A method of treating a disease or disorder in a subject comprising administering to a subject the modified oligonucleotide of any one of embodiments II-1 to II-79, or the pharmaceutical composition of embodiment II-80.

Embodiment II-82. The method of embodiment II-81, wherein the administering to a subject is parenteral, subcutaneous, transdermal, intraocular, intramuscular or intravenous.

Embodiment II-83. The method of any one of embodiments 11-81 to II-82, wherein the subject is human.

Embodiment II-84. The method of any one of embodiments 11-81 to II-82, wherein the subject is a non-human primate.

Embodiment II-85. The method of any one of embodiments II-81 to II-84, wherein the modified oligonucleotide is complementary to PCSK9, and the disease or disorder is a PCSK9-associated disease or disorder.

Embodiment II-86. The method of any one of embodiments II-81 to 84, wherein the modified oligonucleotide is complementary to DMPK, and the disease or disorder is a DMPK-associated disease or disorder.

Embodiment II-87. The method of any one of embodiments 11-81 to II-84, wherein the modified oligonucleotide is complementary to AAT, and the disease or disorder is a AAT-associated disease or disorder.

Embodiment II-88. The method of any one of embodiments 11-81 to II-84, wherein the modified oligonucleotide is complementary to TTR, and the disease or disorder is a TTR-associated disease or disorder.

### Set III

Embodiment III-1. A modified multi-segmented antisense oligonucleotide that is complementary to a nucleotide sequence in a gene transcript, wherein the modified multi-segmented antisense oligonucleotide comprises from 5′ to 3′:

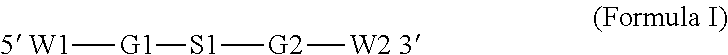

wherein:
   W1 is a 5′ wing segment;
   W2 is a 3′ wing segment;
   G1 is a first gap segment;
   S1 is a first separator segment; and
   G2 is a second gap segment,
wherein each segment is joined by an internucleoside linkage, and
wherein at least one nucleoside of each of W1, W2, and S1 is a modified nucleoside.

Embodiment III-2. The modified multi-segmented antisense oligonucleotide of embodiment III-1, wherein the modified multi-segmented antisense oligonucleotide comprises from 5′ to 3′:

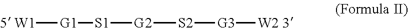

wherein:
S2 is a second separator segment; and
G3 is a third gap segment, wherein at least one nucleoside of S2 is a modified nucleoside.

Embodiment III-3. The modified multi-segmented antisense oligonucleotide of embodiment III-1 or III-2, wherein W1 comprises 2 to 25 linked nucleosides.

Embodiment III-4. The modified multi-segmented antisense oligonucleotide of embodiment III-3, wherein the first 5′ nucleoside of W1 is a modified nucleoside and/or when the last 3′ nucleoside of W1 is a modified nucleoside.

Embodiment III-5. The modified multi-segmented antisense oligonucleotide of any one of embodiments III-1 to III-4, wherein W2 comprises 2 to 35 linked nucleosides.

Embodiment III-6. The modified multi-segmented antisense oligonucleotide of embodiment III-5, wherein the first 5′ nucleoside of W2 is a modified nucleoside and/or when the last 3′ nucleoside of W2 is a modified nucleoside.

Embodiment III-7. The modified multi-segmented antisense oligonucleotide of any one of embodiments III-1 to III-6, wherein G1 comprises 1 to 10 linked deoxynucleosides.

Embodiment III-8. The modified multi-segmented antisense oligonucleotide of any one of embodiments III-1 to III-7, wherein G2 comprises 1 to 10 linked deoxynucleosides.

Embodiment III-9. The modified multi-segmented antisense oligonucleotide of any one of embodiments III-1 to III-8, wherein S1 comprises 1 to 3 linked nucleosides.

Embodiment III-10. The modified multi-segmented antisense oligonucleotide of any one of embodiments III-2 to III-9, wherein G3 comprises 1 to 10 linked deoxynucleosides.

Embodiment III-11. The modified multi-segmented antisense oligonucleotide of any one of embodiments III-2 to III-10, wherein S2 comprises 1 to 3 linked nucleosides.

Embodiment III-12. The modified multi-segmented antisense oligonucleotide of any one of embodiments III-1 to III-11, wherein the modified multi-segmented antisense oligonucleotide is 11-50 nucleobases in length.

Embodiment III-13. The modified multi-segmented antisense oligonucleotide of any one of embodiments III-1 to III-11, wherein the modified multi-segmented antisense oligonucleotide is 11-30 nucleobases in length.

Embodiment III-14. The modified multi-segmented antisense oligonucleotide of any one of embodiments III-1 to III-11, wherein the modified multi-segmented antisense oligonucleotide is at least 20 nucleobases in length.

Embodiment III-15. The modified multi-segmented antisense oligonucleotide of any one of embodiments III-1 to III-11, wherein the modified multi-segmented antisense oligonucleotide is 20 nucleobases in length.

Embodiment III-16. The modified multi-segmented antisense oligonucleotide of embodiment III-15, wherein W1 comprises 2 to 8 linked nucleosides.

Embodiment III-17. The modified multi-segmented antisense oligonucleotide of embodiment III-15, wherein W1 comprises 2 to 6 linked nucleosides.

Embodiment III-18. The modified multi-segmented antisense oligonucleotide of embodiment III-15, wherein W1 comprises 4, 5, or 6 linked nucleosides.

Embodiment III-19. The modified multi-segmented antisense oligonucleotide of embodiment III-15, wherein W1 comprises 5 linked nucleosides.

Embodiment III-20. The modified multi-segmented antisense oligonucleotide of any one of embodiments III-15 to III-19, wherein G1 comprises 2 to 7 linked deoxynucleosides.

Embodiment III-21. The modified multi-segmented antisense oligonucleotide of any one of embodiments III-15 to III-19, wherein G1 comprises 4 linked deoxynucleosides.

Embodiment III-22. The modified multi-segmented antisense oligonucleotide of any one of embodiments III-15 to III-19, wherein G1 comprises 5 linked deoxynucleosides.

Embodiment III-23. The modified multi-segmented antisense oligonucleotide of any one of embodiments III-15 to III-19, wherein G1 comprises 6 linked deoxynucleosides.

Embodiment III-24. The modified multi-segmented antisense oligonucleotide of any one of embodiments III-15 to III-23, wherein G2 comprises 1 to 7 linked deoxynucleosides.

Embodiment III-25. The modified multi-segmented antisense oligonucleotide of any one of embodiments III-15 to III-23, wherein G2 comprises 2 to 7 linked deoxynucleosides.

Embodiment III-26. The modified multi-segmented antisense oligonucleotide of any one of embodiments III-15 to III-23, wherein G2 comprises 1 to 2 linked deoxynucleosides.

Embodiment III-27. The modified multi-segmented antisense oligonucleotide of any one of embodiments III-15 to III-23, wherein G2 comprises 4 linked deoxynucleosides.

Embodiment III-28. The modified multi-segmented antisense oligonucleotide of any one of embodiments III-15 to III-23, wherein G2 comprises 5 linked deoxynucleosides.

Embodiment III-29. The modified multi-segmented antisense oligonucleotide of any one of embodiments III-15 to III-23, wherein G2 comprises 6 linked deoxynucleosides.

Embodiment III-30. The modified multi-segmented antisense oligonucleotide of any one of embodiments III-15 to III-23, wherein G2 comprises 7 linked deoxynucleosides.

Embodiment III-31. The modified multi-segmented antisense oligonucleotide of any one of embodiments III-15 to III-30, wherein G3 comprises 2 to 7 linked deoxynucleosides.

Embodiment III-32. The modified multi-segmented antisense oligonucleotide of any one of embodiments III-15 to III-30, wherein G3 comprises 4 linked deoxynucleosides.

Embodiment III-33. The modified multi-segmented antisense oligonucleotide of any one of embodiments III-15 to III-30, wherein G3 comprises 5 linked deoxynucleosides.

Embodiment III-34. The modified multi-segmented antisense oligonucleotide of any one of embodiments III-15 to III-30, wherein G3 comprises 6 linked deoxynucleosides.

Embodiment III-35. The modified multi-segmented antisense oligonucleotide of any one of embodiments III-15 to III-30, wherein G3 comprises 7 linked deoxynucleosides.

Embodiment III-36. The modified multi-segmented antisense oligonucleotide of any one of embodiments III-15 to III-35, wherein S1 comprises at least one linked nucleoside.

Embodiment III-37. The modified multi-segmented antisense oligonucleotide of any one of embodiments III-15 to III-35, wherein S1 comprises one linked nucleoside.

Embodiment III-38. The modified multi-segmented antisense oligonucleotide of any one of embodiments III-15 to III-35, wherein S1 comprises 2 linked nucleosides.

Embodiment III-39. The modified multi-segmented antisense oligonucleotide of any one of embodiments III-15 to III-38, wherein S2 comprises at least one linked nucleoside.

Embodiment III-40. The modified multi-segmented antisense oligonucleotide of any one of embodiments III-15 to III-38, wherein S2 comprises one linked nucleoside.

Embodiment III-41. The modified multi-segmented antisense oligonucleotide of any one of embodiments III-15 to III-38, wherein S2 comprises 2 linked nucleosides.

Embodiment III-42. The modified multi-segmented antisense oligonucleotide of any one of embodiments III-15 to III-41, wherein W2 comprises 2 to 8 linked nucleosides.

Embodiment III-43. The modified multi-segmented antisense oligonucleotide of any one of embodiments III-15 to III-41, wherein W2 comprises 2 to 6 linked nucleosides.

Embodiment III-44. The modified multi-segmented antisense oligonucleotide of any one of embodiments III-15 to III-41, wherein W2 comprises 3 linked nucleosides.

Embodiment III-45. The modified multi-segmented antisense oligonucleotide of any one of embodiments III-15 to III-41, wherein W2 comprises 4 linked nucleosides.

Embodiment III-46. The modified multi-segmented antisense oligonucleotide of any one of embodiments III-15 to III-41, wherein W2 comprises 5 linked nucleosides.

Embodiment III-47. The modified multi-segmented antisense oligonucleotide of any one of embodiments III-15 to III-41, wherein W2 comprises 6 linked nucleosides.

Embodiment III-48. The modified multi-segmented antisense oligonucleotide of any one of embodiments III-15 to III-47, wherein the S1 is at position 10 of the modified multi-segmented antisense oligonucleotide.

Embodiment III-49. The modified multi-segmented antisense oligonucleotide of any one of embodiments III-15 to III-47, wherein the S1 is at position 11 of the modified multi-segmented antisense oligonucleotide.

Embodiment III-50. The modified multi-segmented antisense oligonucleotide of embodiment III-48, wherein:
W1 comprises 4 linked nucleosides,
G1 comprises 5 linked deoxynucleosides,
S1 comprises 1 linked nucleoside,
G2 comprises 5 linked deoxynucleosides, and
W2 comprises 5 linked nucleosides.

Embodiment III-51. The modified multi-segmented antisense oligonucleotide of embodiment III-48, wherein
W1 comprises 5 linked nucleosides,
G1 comprises 4 linked deoxynucleosides,
S1 comprises 1 linked nucleoside,
G2 comprises 5 linked deoxynucleosides, and
W2 comprises 5 linked nucleosides.

Embodiment III-52. The modified multi-segmented antisense oligonucleotide of embodiment III-1, wherein:
W1 comprises 2 to 8 linked nucleosides,
G1 comprises 2 to 7 linked deoxynucleosides,
S1 comprises 1 to 2 linked nucleosides,
G2 comprises 2 to 7 linked deoxynucleosides, and
W2 comprises 2 to 8 linked nucleosides.

Embodiment III-53. The modified multi-segmented antisense oligonucleotide of embodiment III-52, wherein:
W1 comprises 2 to 6 linked nucleosides,
G1 comprises 2 to 7 linked deoxynucleosides,
S1 comprises 1 linked nucleoside,
G2 comprises 2 to 7 linked deoxynucleosides, and
W2 comprises 2 to 6 linked nucleosides.

Embodiment III-54. The modified multi-segmented antisense oligonucleotide of embodiment III-2, wherein:
W1 comprises 2 to 8 linked nucleosides,
G1 comprises 2 to 7 linked deoxynucleosides,
S1 comprises 1 linked nucleoside,
G2 comprises 1 to 2 linked deoxynucleosides,
S2 comprises 1 linked nucleoside,
G3 comprises 2 to 7 linked deoxynucleosides, and
W2 comprises 2 to 8 linked nucleosides.

Embodiment III-55. The modified multi-segmented antisense oligonucleotide of embodiment III-54, wherein:
W1 comprises 2 to 6 linked nucleosides,
G1 comprises 2 to 7 linked deoxynucleosides,
S1 comprises 1 linked nucleoside,
G2 comprises 1 to 2 linked deoxynucleosides,
S2 comprises 1 linked nucleoside,
G3 comprises 2 to 7 linked deoxynucleosides, and
W2 comprises 2 to 6 linked nucleosides.

Embodiment III-56. The modified multi-segmented antisense oligonucleotide of any one of embodiments III-52 to III-53, wherein the length of the G1-S1-G2 segment is between 8 and 12 nucleobases in length.

Embodiment III-57. The modified multi-segmented antisense oligonucleotide of any one of embodiments III-54 to III-54, wherein the length of the G1-S1-G2-S2-G3 is between 8 and 12 nucleobases in length.

Embodiment III-58. The modified multi-segmented antisense oligonucleotide of any one of embodiments III-1 to III-57, wherein G1, G2, and/or G3, comprises a nucleoside comprising a modification to a 2′-deoxynucleoside.

Embodiment III-59. The modified multi-segmented antisense oligonucleotide of embodiment III-58, wherein G1, G2, and/or G3, comprises a nucleoside comprising a 2′-deoxy 5-methylcytidine sugar modification.

Embodiment III-60. The modified multi-segmented antisense oligonucleotide of any one of embodiments III-1 to III-59, wherein S1 and/or S2, comprises a nucleoside comprising a 2′-O-methoxyethyl sugar modification.

Embodiment III-61. The modified multi-segmented antisense oligonucleotide of any one of embodiments III-1 to III-59, wherein S1 and/or S2 comprises a nucleoside comprising a 5-methylcytidine.

Embodiment III-62. The modified multi-segmented antisense oligonucleotide of any one of embodiments III-1 to III-59, wherein S1 and/or S2 comprises a nucleoside comprising a 2′-O-methyl sugar modification.

Embodiment III-63. The modified multi-segmented antisense oligonucleotide of any one of embodiments III-1 to III-59, wherein S1 and/or S2 comprises a nucleoside comprising a 2′-O-ethyl sugar modification.

Embodiment III-64. The modified multi-segmented antisense oligonucleotide of any one of embodiments III-1 to III-63, wherein S1 and/or S2 comprises a nucleoside comprising a 2′-OH sugar modification.

Embodiment III-65. The modified multi-segmented antisense oligonucleotide of any one of embodiments III-1 to III-64, wherein S1 and/or S2 comprises a nucleoside comprising a 2′-fluoro sugar modification.

Embodiment III-66. The modified multi-segmented antisense oligonucleotide of any one of embodiments III-1 to III-65, wherein S1 and/or S2 comprises a nucleoside comprising a 2′-fluoro-arabinonucleic acid (2′-fluoro-ANA) sugar modification.

Embodiment III-67. The modified multi-segmented antisense oligonucleotide of any one of embodiments III-1 to III-66, wherein S1 and/or S2 comprises a glycol nucleic acid (GNA).

Embodiment III-68. The modified multi-segmented antisense oligonucleotide of any one of embodiments III-1 to III-67, wherein S1 and/or S2 comprises a locked nucleic acid (LNA).

Embodiment III-69. The modified multi-segmented antisense oligonucleotide of any one of embodiments III-1 to III-68, wherein W1 comprises a nucleoside comprising a 2′-deoxy sugar modification.

Embodiment III-70. The modified multi-segmented antisense oligonucleotide of any one of embodiments III-1 to III-69, wherein W1 comprises a nucleoside comprising a 2′-O-methoxyethyl sugar modification.

Embodiment III-71. The modified multi-segmented antisense oligonucleotide of any one of embodiments III-1 to III-70, wherein W1 comprises a nucleoside comprising a 2′-O-methyl sugar modification.

Embodiment III-72. The modified multi-segmented antisense oligonucleotide of embodiment III-71, wherein W1 comprises a 2′-O-methyl 5-methylcytidine.

Embodiment III-73. The modified multi-segmented antisense oligonucleotide of any one of embodiments III-1 to III-71, wherein W1 comprises a nucleoside comprising a 2′-fluoro sugar modification.

Embodiment III-74. The modified multi-segmented antisense oligonucleotide of any one of embodiments III-1 to III-73, wherein W1 comprises a nucleoside comprising a 2′-fluoro-arabinonucleic acid (2′-fluoro-ANA) modification.

Embodiment III-75. The modified multi-segmented antisense oligonucleotide of any one of embodiments III-1 to III-74, wherein W1 comprises a glycol nucleic acid (GNA).

Embodiment III-76. The modified multi-segmented antisense oligonucleotide of any one of embodiments III-1 to III-75, wherein W1 comprises a locked nucleic acid (LNA).

Embodiment III-77. The modified multi-segmented antisense oligonucleotide of any one of embodiments III-1 to III-76, wherein W1 comprises a nucleoside comprising a 2′-O-ethyl sugar modification.

Embodiment III-78. The modified multi-segmented antisense oligonucleotide of any one of embodiments III-1 to III-77, wherein W2 comprises a nucleoside comprising a 2′-deoxy sugar modification.

Embodiment III-79. The modified multi-segmented antisense oligonucleotide of any one of embodiments III-1 to III-78, wherein W2 comprises a nucleoside comprising a 2′-O-methoxyethyl sugar modification.

Embodiment III-80. The modified multi-segmented antisense oligonucleotide of embodiment III-79, wherein W2 comprises a 2′-O-methoxyethyl 5-methylcytidine.

Embodiment III-81. The modified multi-segmented antisense oligonucleotide of any one of embodiments III-1 to III-78, wherein W2 comprises a nucleoside comprising a 2′-fluoro sugar modification.

Embodiment III-82. The modified multi-segmented antisense oligonucleotide of embodiment III-81, wherein W2 comprises a nucleoside comprising a 2′-fluoro-arabinonucleic acid (2′-fluoro-ANA) modification.

Embodiment III-83. The modified multi-segmented antisense oligonucleotide of any one of embodiments III-1 to III-82, wherein W2 comprises a glycol nucleic acid (GNA).

Embodiment III-84. The modified multi-segmented antisense oligonucleotide of any one of embodiments III-1 to III-83, wherein W2 comprises a locked nucleic acid (LNA).

Embodiment III-85. The modified multi-segmented antisense oligonucleotide of any one of embodiments III-1 to III-84, wherein W2 comprises a nucleoside comprising a 2′-O-ethyl sugar modification.

Embodiment III-86. The modified multi-segmented antisense oligonucleotide of any one of embodiments III-1 to III-85, wherein at least one internucleoside linkage is an phosphodiester linkage.

Embodiment III-87. The modified multi-segmented antisense oligonucleotide of any one of embodiments III-1 to III-86, wherein at least one internucleoside linkage is a modified internucleoside linkage.

Embodiment III-88. The modified multi-segmented antisense oligonucleotide of embodiment III-87, wherein the modified internucleoside linkage is a phosphorothioate internucleoside linkage.

Embodiment III-89. The modified multi-segmented antisense oligonucleotide of embodiment III-87, wherein the modified internucleoside linkage comprises a phosphorodiamidate morpholino modification.

Embodiment III-90. The modified multi-segmented antisense oligonucleotide of any one of embodiments III-1 to III-89, wherein:
a. a ratio of EC₅₀ of cells treated with a modified multi-segmented antisense oligonucleotide/EC₅₀ of cells treated with a modified antisense oligonucleotide with the same template nucleobase sequence that does not comprise a separator segment is about 0.05 to about 250;
b. a MTT CC25 value of cells treated with a modified multi-segmented antisense oligonucleotide is about 10 nM to about 250 nM;
c. a CCK8 CC30 value of cells treated with a modified multi-segmented antisense oligonucleotide is about 10 nM to about 250 nM;
d. a ratio of Fold CC30/Fold EC50 of cells treated with a modified multi-segmented antisense oligonucleotide compared to a modified antisense oligonucleotide with the same template nucleobase sequence that does not comprise a separator segment is about is about 0.01 to about 50;
e. exhibits an at least 1.1 fold decrease in melting temperature, compared to a modified antisense oligonucleotide with the same template nucleobase sequence that does not comprise a separator segment; and/or
f. exhibits an at least 1.1 fold increase in recruitment of an endonuclease, compared to modified antisense oligonucleotide with the same template nucleobase sequence that does not comprise a separator segment.

Embodiment III-91. The modified multi-segmented antisense oligonucleotide of any one of embodiments III-1 to III-90, wherein the modified multi-segmented antisense oligonucleotide is complementary to a nucleotide sequence in a viral gene transcript, optionally wherein the viral gene is selected from the group consisting of a HPV, HBV, EBV, HSV, HIV, and a RSV gene.

Embodiment III-92. The modified multi-segmented antisense oligonucleotide of any one of embodiments III-1 to III-90, wherein the modified multi-segmented antisense oligonucleotide is complementary to a human gene transcript.

Embodiment III-93. The modified multi-segmented antisense oligonucleotide of embodiment III-92 wherein the modified multi-segmented antisense oligonucleotide is complementary to a human gene transcript selected from the group consisting of DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) and a hydroxy acid oxidase 1 gene transcript.

Embodiment III-94. The modified multi-segmented antisense oligonucleotide of any one of embodiments III-1 to III-90, wherein the modified multi-segmented antisense oligonucleotide is complementary to a gene transcript selected from the group consisting of DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) or hydroxy acid oxidase 1, Matla, (pro)renin receptor/(P)RR, COVID-19 5′UTR, Acc1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 150 Dystrophin, Exon 151 Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, H1N1, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2a, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF1, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, and YAP1.

Embodiment III-95. The modified multi-segmented antisense oligonucleotide of any one of embodiments III-1 to III-90, wherein the modified multi-segmented antisense oligonucleotide is complementary to gene transcript selected from the group consisting of an oncogenic gene transcript, an inflammatory gene transcript, a metabolic disease gene transcript, a cardiovascular gene transcript, a liver disease gene transcript, an infectious disease gene transcript, a neurological disease gene transcript, a neuromuscular disease gene transcript, an eye disease gene transcript, a kidney disease gene transcript, a respiratory disease gene transcript, a blood disease gene transcript, a wound healing gene transcript, a transplantation gene transcript, an autoimmune disease gene transcript, and a neuropsychiatric gene transcript.

Embodiment III-96. A modified multi-segmented antisense oligonucleotide comprising the structure of the modified multi-segmented antisense oligonucleotide of any one of SEQ ID NOS: 11-1453 or 5000-30983.

Embodiment III-97. A modified multi-segmented antisense oligonucleotide comprising the structure of the modified multi-segmented antisense oligonucleotide of any one of SEQ ID NOS: 11-1453 or 5000-30983, or an oligonucleotide having at least 70%, at least 80%, at least 90%, at least 95%, or at least 99% sequence identity thereto.

Embodiment III-98. A modified multi-segmented antisense oligonucleotide comprising the structure of the modified multi-segmented antisense oligonucleotide of any one of SEQ ID NOS: 11-1453 or 5000-30983, or an oligonucleotide comprising at least 1, 2, 3, 4 or 5 further modifications thereto.

Embodiment III-99. A pharmaceutical composition comprising the modified multi-segmented antisense oligonucleotide of any one of embodiments III-1 to III-98, or salt thereof and at least one pharmaceutically acceptable carrier or diluent.

Embodiment III-100. A method of treating a disease, disorder, or condition comprising administering to a subject the modified multi-segmented antisense oligonucleotide of any one of embodiments III-1 to III-98, or the pharmaceutical composition of embodiment III-99.

Embodiment III-101. The method of embodiment III-100, wherein the administering to a subject is parenteral.

Embodiment III-102. The method of embodiment III-100, wherein the administering to a subject is subcutaneous.

Embodiment III-103. The method of embodiment III-100, wherein the administering to a subject is transdermal.

Embodiment III-104. The method of embodiment III-100, wherein the administering to a subject is intraocular.

Embodiment III-105. The method of embodiment III-100, wherein the administering to a subject is intramuscular.

Embodiment III-106. The method of embodiment III-100, wherein the administering to a subject is intravenous.

Embodiment III-107. The method of any one of embodiments III-100 to III-106, wherein the subject is human.

Embodiment III-108. The method of any one of embodiments III-100 to III-106, wherein the subject is a non-human primate.

What is claimed is:

1. A modified multi-segmented antisense oligonucleotide that is complementary to a nucleotide sequence in a gene transcript, wherein the modified multi-segmented antisense oligonucleotide comprises from 5′ to 3′ 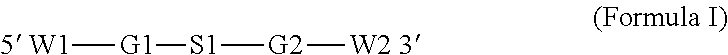
wherein:
W1 is a 5′ wing segment;
W2 is a 3′ wing segment;
G1 is a first gap segment;
S1 is a first separator segment; and
G2 is a second gap segment,
wherein each segment is joined by an internucleoside linkage, and
wherein at least one nucleoside of each of W1, W2, and S1 is a modified nucleoside.

2. The modified multi-segmented antisense oligonucleotide of claim 1, wherein the modified multi-segmented antisense oligonucleotide comprises from 5′ to 3′: 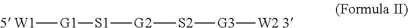
wherein:
S2 is a second separator segment; and
G3 is a third gap segment, wherein at least one nucleoside of S2 is a modified nucleoside.

3. The modified multi-segmented antisense oligonucleotide of claim 1 or claim 2, wherein W1 comprises 2 to 25 linked nucleosides.

4. The modified multi-segmented antisense oligonucleotide of claim 3, wherein the first 5′ nucleoside of W1 is a modified nucleoside and/or when the last 3′ nucleoside of W1 is a modified nucleoside.

5. The modified multi-segmented antisense oligonucleotide of any one of claims 1 to 4, wherein W2 comprises 2 to 35 linked nucleosides.

6. The modified multi-segmented antisense oligonucleotide of claim 5, wherein the first 5′ nucleoside of W2 is a modified nucleoside and/or when the last 3′ nucleoside of W2 is a modified nucleoside.

7. The modified multi-segmented antisense oligonucleotide of any one of claims 1 to 6, wherein G1 comprises 1 to 10 linked deoxynucleosides.

8. The modified multi-segmented antisense oligonucleotide of any one of claims 1 to 7, wherein G2 comprises 1 to 10 linked deoxynucleosides.

9. The modified multi-segmented antisense oligonucleotide of any one of claims 1 to 8, wherein S1 comprises 1 to 3 linked nucleosides.

10. The modified multi-segmented antisense oligonucleotide of any one of claims 2 to 9, wherein G3 comprises 1 to 10 linked deoxynucleosides.

11. The modified multi-segmented antisense oligonucleotide of any one of claims 2 to 10, wherein S2 comprises 1 to 3 linked nucleosides.

12. The modified multi-segmented antisense oligonucleotide of any one of claims 1 to 11, wherein the modified multi-segmented antisense oligonucleotide is 11-50 nucleobases in length.

13. The modified multi-segmented antisense oligonucleotide of any one of claims 1 to 11, wherein the modified multi-segmented antisense oligonucleotide is 11-30 nucleobases in length.

14. The modified multi-segmented antisense oligonucleotide of any one of claims 1 to 11, wherein the modified multi-segmented antisense oligonucleotide is at least 20 nucleobases in length.

15. The modified multi-segmented antisense oligonucleotide of any one of claims 1 to 11, wherein the modified multi-segmented antisense oligonucleotide is 20 nucleobases in length.

16. The modified multi-segmented antisense oligonucleotide of claim 15, wherein W1 comprises 2 to 8 linked nucleosides.

17. The modified multi-segmented antisense oligonucleotide of claim 15, wherein W1 comprises 2 to 6 linked nucleosides.

18. The modified multi-segmented antisense oligonucleotide of claim 15, wherein W1 comprises 4, 5, or 6 linked nucleosides.

19. The modified multi-segmented antisense oligonucleotide of claim 15, wherein W1 comprises 5 linked nucleosides.

20. The modified multi-segmented antisense oligonucleotide of any one of claims 15 to 19, wherein G1 comprises 2 to 7 linked deoxynucleosides.

21. The modified multi-segmented antisense oligonucleotide of any one of claims 15 to 19, wherein G1 comprises 4 linked deoxynucleosides.

22. The modified multi-segmented antisense oligonucleotide of any one of claims 15 to 19, wherein G1 comprises 5 linked deoxynucleosides.

23. The modified multi-segmented antisense oligonucleotide of any one of claims 15 to 19, wherein G1 comprises 6 linked deoxynucleosides.

24. The modified multi-segmented antisense oligonucleotide of any one of claims 15 to 23, wherein G2 comprises 1 to 7 linked deoxynucleosides.

25. The modified multi-segmented antisense oligonucleotide of any one of claims 15 to 23, wherein G2 comprises 2 to 7 linked deoxynucleosides.

26. The modified multi-segmented antisense oligonucleotide of any one of claims 15 to 23, wherein G2 comprises 1 to 2 linked deoxynucleosides.

27. The modified multi-segmented antisense oligonucleotide of any one of claims 15 to 23, wherein G2 comprises 4 linked deoxynucleosides.

28. The modified multi-segmented antisense oligonucleotide of any one of claims 15 to 23, wherein G2 comprises 5 linked deoxynucleosides.

29. The modified multi-segmented antisense oligonucleotide of any one of claims 15 to 23, wherein G2 comprises 6 linked deoxynucleosides.

30. The modified multi-segmented antisense oligonucleotide of any one of claims 15 to 23, wherein G2 comprises 7 linked deoxynucleosides.

31. The modified multi-segmented antisense oligonucleotide of any one of claims 15 to 30, wherein G3 comprises 2 to 7 linked deoxynucleosides.

32. The modified multi-segmented antisense oligonucleotide of any one of claims 15 to 30, wherein G3 comprises 4 linked deoxynucleosides.

33. The modified multi-segmented antisense oligonucleotide of any one of claims 15 to 30, wherein G3 comprises 5 linked deoxynucleosides.

34. The modified multi-segmented antisense oligonucleotide of any one of claims 15 to 30, wherein G3 comprises 6 linked deoxynucleosides.

35. The modified multi-segmented antisense oligonucleotide of any one of claims 15 to 30, wherein G3 comprises 7 linked deoxynucleosides.

36. The modified multi-segmented antisense oligonucleotide of any one of claims 15 to 35, wherein S1 comprises at least one linked nucleoside.

37. The modified multi-segmented antisense oligonucleotide of any one of claims 15 to 35, wherein S1 comprises one linked nucleoside.

38. The modified multi-segmented antisense oligonucleotide of any one of claims 15 to 35, wherein S1 comprises 2 linked nucleosides.

39. The modified multi-segmented antisense oligonucleotide of any one of claims 15 to 38, wherein S2 comprises at least one linked nucleoside.

40. The modified multi-segmented antisense oligonucleotide of any one of claims 15 to 38, wherein S2 comprises one linked nucleoside.

41. The modified multi-segmented antisense oligonucleotide of any one of claims 15 to 38, wherein S2 comprises 2 linked nucleosides.

42. The modified multi-segmented antisense oligonucleotide of any one of claims 15 to 41, wherein W2 comprises 2 to 8 linked nucleosides.

43. The modified multi-segmented antisense oligonucleotide of any one of claims 15 to 41, wherein W2 comprises 2 to 6 linked nucleosides.

44. The modified multi-segmented antisense oligonucleotide of any one of claims 15 to 41, wherein W2 comprises 3 linked nucleosides.

45. The modified multi-segmented antisense oligonucleotide of any one of claims 15 to 41, wherein W2 comprises 4 linked nucleosides.

46. The modified multi-segmented antisense oligonucleotide of any one of claims 15 to 41, wherein W2 comprises 5 linked nucleosides.

47. The modified multi-segmented antisense oligonucleotide of any one of claims 15 to 41, wherein W2 comprises 6 linked nucleosides.

48. The modified multi-segmented antisense oligonucleotide of any one of claims 15 to 47, wherein the S1 is at position 10 of the modified multi-segmented antisense oligonucleotide.

49. The modified multi-segmented antisense oligonucleotide of any one of claims 15 to 47, wherein the S1 is at position 11 of the modified multi-segmented antisense oligonucleotide.

50. The modified multi-segmented antisense oligonucleotide of claim 48, wherein:
W1 comprises 4 linked nucleosides,
G1 comprises 5 linked deoxynucleosides,
S1 comprises 1 linked nucleoside,
G2 comprises 5 linked deoxynucleosides, and
W2 comprises 5 linked nucleosides.

51. The modified multi-segmented antisense oligonucleotide of claim 48, wherein
W1 comprises 5 linked nucleosides,
G1 comprises 4 linked deoxynucleosides,
S1 comprises 1 linked nucleoside,
G2 comprises 5 linked deoxynucleosides, and
W2 comprises 5 linked nucleosides.

52. The modified multi-segmented antisense oligonucleotide of claim 1, wherein:
W1 comprises 2 to 8 linked nucleosides,
G1 comprises 2 to 7 linked deoxynucleosides,
S1 comprises 1 to 2 linked nucleosides,
G2 comprises 2 to 7 linked deoxynucleosides, and
W2 comprises 2 to 8 linked nucleosides.

53. The modified multi-segmented antisense oligonucleotide of claim 52, wherein:
W1 comprises 2 to 6 linked nucleosides,
G1 comprises 2 to 7 linked deoxynucleosides,
S1 comprises 1 linked nucleoside,
G2 comprises 2 to 7 linked deoxynucleosides, and
W2 comprises 2 to 6 linked nucleosides.

54. The modified multi-segmented antisense oligonucleotide of claim 2, wherein:
W1 comprises 2 to 8 linked nucleosides,
G1 comprises 2 to 7 linked deoxynucleosides,
S1 comprises 1 linked nucleoside,
G2 comprises 1 to 2 linked deoxynucleosides,
S2 comprises 1 linked nucleoside,
G3 comprises 2 to 7 linked deoxynucleosides, and
W2 comprises 2 to 8 linked nucleosides.

55. The modified multi-segmented antisense oligonucleotide of claim 54, wherein:
W1 comprises 2 to 6 linked nucleosides,
G1 comprises 2 to 7 linked deoxynucleosides,
S1 comprises 1 linked nucleoside,
G2 comprises 1 to 2 linked deoxynucleosides,
S2 comprises 1 linked nucleoside,
G3 comprises 2 to 7 linked deoxynucleosides, and
W2 comprises 2 to 6 linked nucleosides.

56. The modified multi-segmented antisense oligonucleotide of any one of claims 52-53, wherein the length of the G1-S1-G2 segment is between 8 and 12 nucleobases in length.

57. The modified multi-segmented antisense oligonucleotide of any one of claims 54-54, wherein the length of the G1-S1-G2-S2-G3 is between 8 and 12 nucleobases in length.

58. The modified multi-segmented antisense oligonucleotide of any one of claims 1 to 57, wherein G1, G2, and/or G3, comprises a nucleoside comprising a modification to a 2′-deoxynucleoside.

59. The modified multi-segmented antisense oligonucleotide of claim 58, wherein G1, G2, and/or G3, comprises a nucleoside comprising a 2′-deoxy 5-methylcytidine sugar modification.

60. The modified multi-segmented antisense oligonucleotide of any one of claims 1 to 59, wherein S1 and/or S2, comprises a nucleoside comprising a 2′-O-methoxyethyl sugar modification.

61. The modified multi-segmented antisense oligonucleotide of any one of claims 1 to 59, wherein S1 and/or S2 comprises a nucleoside comprising a 5-methylcytidine.

62. The modified multi-segmented antisense oligonucleotide of any one of claims 1 to 59, wherein S1 and/or S2 comprises a nucleoside comprising a 2′-O-methyl sugar modification.

63. The modified multi-segmented antisense oligonucleotide of any one of claims 1 to 59, wherein S1 and/or S2 comprises a nucleoside comprising a 2′-O-ethyl sugar modification.

64. The modified multi-segmented antisense oligonucleotide of any one of claims 1 to 63, wherein S1 and/or S2 comprises a nucleoside comprising a 2′-OH sugar modification.

65. The modified multi-segmented antisense oligonucleotide of any one of claims 1 to 64, wherein S1 and/or S2 comprises a nucleoside comprising a 2′-fluoro sugar modification.

66. The modified multi-segmented antisense oligonucleotide of any one of claims 1 to 65, wherein S1 and/or S2 comprises a nucleoside comprising a 2′-fluoro-arabinonucleic acid (2′-fluoro-ANA) sugar modification.

67. The modified multi-segmented antisense oligonucleotide of any one of claims 1 to 66, wherein S1 and/or S2 comprises a glycol nucleic acid (GNA).

68. The modified multi-segmented antisense oligonucleotide of any one of claims 1 to 67, wherein S1 and/or S2 comprises a locked nucleic acid (LNA).

69. The modified multi-segmented antisense oligonucleotide of any one of claims 1 to 68, wherein W1 comprises a nucleoside comprising a 2′-deoxy sugar modification.

70. The modified multi-segmented antisense oligonucleotide of any one of claims 1 to 69, wherein W1 comprises a nucleoside comprising a 2′-O-methoxyethyl sugar modification.

71. The modified multi-segmented antisense oligonucleotide of any one of claims 1 to 70, wherein W1 comprises a nucleoside comprising a 2′-O-methyl sugar modification.

72. The modified multi-segmented antisense oligonucleotide of claim 71, wherein W1 comprises a 2′-O-methyl 5-methylcytidine.

73. The modified multi-segmented antisense oligonucleotide of any one of claims 1 to 71, wherein W1 comprises a nucleoside comprising a 2′-fluoro sugar modification.

74. The modified multi-segmented antisense oligonucleotide of any one of claims 1 to 73, wherein W1 comprises a nucleoside comprising a 2′-fluoro-arabinonucleic acid (2′-fluoro-ANA) modification.

75. The modified multi-segmented antisense oligonucleotide of any one of claims 1 to 74, wherein W1 comprises a glycol nucleic acid (GNA).

76. The modified multi-segmented antisense oligonucleotide of any one of claims 1 to 75, wherein W1 comprises a locked nucleic acid (LNA).

77. The modified multi-segmented antisense oligonucleotide of any one of claims 1 to 76, wherein W1 comprises a nucleoside comprising a 2′-O-ethyl sugar modification.

78. The modified multi-segmented antisense oligonucleotide of any one of claims 1 to 77, wherein W2 comprises a nucleoside comprising a 2′-deoxy sugar modification.

79. The modified multi-segmented antisense oligonucleotide of any one of claims 1 to 78, wherein W2 comprises a nucleoside comprising a 2′-O-methoxyethyl sugar modification.

80. The modified multi-segmented antisense oligonucleotide of claim 79, wherein W2 comprises a 2′-O-methoxyethyl 5-methylcytidine.

81. The modified multi-segmented antisense oligonucleotide of any one of claims 1 to 78, wherein W2 comprises a nucleoside comprising a 2′-fluoro sugar modification.

82. The modified multi-segmented antisense oligonucleotide of claim 81, wherein W2 comprises a nucleoside comprising a 2′-fluoro-arabinonucleic acid (2′-fluoro-ANA) modification.

83. The modified multi-segmented antisense oligonucleotide of any one of claims 1 to 82, wherein W2 comprises a glycol nucleic acid (GNA).

84. The modified multi-segmented antisense oligonucleotide of any one of claims 1 to 83, wherein W2 comprises a locked nucleic acid (LNA).

85. The modified multi-segmented antisense oligonucleotide of any one of claims 1 to 84, wherein W2 comprises a nucleoside comprising a 2′-O-ethyl sugar modification.

86. The modified multi-segmented antisense oligonucleotide of any one of claims 1 to 85, wherein at least one internucleoside linkage is an phosphodiester linkage.

87. The modified multi-segmented antisense oligonucleotide of any one of claims 1 to 86, wherein at least one internucleoside linkage is a modified internucleoside linkage.

[symbol]

88. The modified multi-segmented antisense oligonucleotide of claim 87, wherein the modified internucleoside linkage is a phosphorothioate internucleoside linkage.

89. The modified multi-segmented antisense oligonucleotide of claim 87, wherein the modified internucleoside linkage comprises a phosphorodiamidate morpholino modification.

90. The modified multi-segmented antisense oligonucleotide of any one of claims 1-89, wherein:
a. a ratio of EC₅₀ of cells treated with a modified multi-segmented antisense oligonucleotide/EC₅₀ of cells treated with a modified antisense oligonucleotide with the same template nucleobase sequence that does not comprise a separator segment is about 0.05 to about 250;
b. a MTT CC25 value of cells treated with a modified multi-segmented antisense oligonucleotide is about 10 nM to about 250 nM;
c. a CCK8 CC30 value of cells treated with a modified multi-segmented antisense oligonucleotide is about 10 nM to about 250 nM;
d. a ratio of Fold CC30/Fold EC50 of cells treated with a modified multi-segmented antisense oligonucleotide compared to a modified antisense oligonucleotide with the same template nucleobase sequence that does not comprise a separator segment is about is about 0.01 to about 50;
e. exhibits an at least 1.1 fold decrease in melting temperature, compared to a modified antisense oligonucleotide with the same template nucleobase sequence that does not comprise a separator segment; and/or
f. exhibits an at least 1.1 fold increase in recruitment of an endonuclease, compared to modified antisense oligonucleotide with the same template nucleobase sequence that does not comprise a separator segment.

91. The modified multi-segmented antisense oligonucleotide of any one of claims 1-90, wherein the modified multi-segmented antisense oligonucleotide is complementary to a nucleotide sequence in a viral gene transcript, optionally wherein the viral gene is selected from the group consisting of a HPV, HBV, EBV, HSV, HIV, and a RSV gene.

92. The modified multi-segmented antisense oligonucleotide of any one of claims 1-90, wherein the modified multi-segmented antisense oligonucleotide is complementary to a human gene transcript.

93. The modified multi-segmented antisense oligonucleotide of claim 92 wherein the modified multi-segmented antisense oligonucleotide is complementary to a human gene transcript selected from the group consisting of DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) and a hydroxy acid oxidase 1 gene transcript.

94. The modified multi-segmented antisense oligonucleotide of any one of claims 1-90, wherein the modified multi-segmented antisense oligonucleotide is complementary to a gene transcript selected from the group consisting of DM1 protein kinase (DMPK), alpha-1 antitrypsin (AAT), Transthyretin (TTR) Proprotein convertase subtilisin/kexin type 9 (PCSK9), apolipoprotein B (ApoB), apolipoprotein C—III (ApoCIII), TNF-alpha, SERPINA8 (AGT), complement factor B (CFB), Diacylglycerol O-Acyltransferase 2 (DGAT2), Patatin Like Phospholipase Domain Containing 3 (PNPLA3), 5′-Aminolevulinate Synthase 1 (ALAS1) or hydroxy acid oxidase 1, Matla, (pro)renin receptor/(P)RR, COVID-19 5′UTR, Acc1, Acc2, ACE2, Androgen receptor, ApoB, ASGR1, ASO, ATXN2, B1AR, B2AR, C9ORF72, Caspase 2, CD19, CD4, Chikungunya Virus, CLPro, Complement component 5, COVID19, COX2, CTGF, DGAT2, DMD, DMPK, DNM2, DUX4, E2 gene, EGFR, Envelope, EphA2, Epithelial sodium channel a subunit, Exon 100 Dystrophin, Exon 101 Dystrophin, Exon 102 Dystrophin, Exon 103 Dystrophin, Exon 104 Dystrophin, Exon 105 Dystrophin, Exon 106 Dystrophin, Exon 107 Dystrophin, Exon 108 Dystrophin, Exon 109 Dystrophin, Exon 110 Dystrophin, Exon 111 Dystrophin, Exon 112 Dystrophin, Exon 113 Dystrophin, Exon 114 Dystrophin, Exon 115 Dystrophin, Exon 116 Dystrophin, Exon 117 Dystrophin, Exon 118 Dystrophin, Exon 119 Dystrophin, Exon 120 Dystrophin, Exon 121 Dystrophin, Exon 122 Dystrophin, Exon 123 Dystrophin, Exon 124 Dystrophin, Exon 125 Dystrophin, Exon 126 Dystrophin, Exon 127 Dystrophin, Exon 128 Dystrophin, Exon 129 Dystrophin, Exon 130 Dystrophin, Exon 131 Dystrophin, Exon 132 Dystrophin, Exon 133 Dystrophin, Exon 134 Dystrophin, Exon 135 Dystrophin, Exon 136 Dystrophin, Exon 137 Dystrophin, Exon 138 Dystrophin, Exon 139 Dystrophin, Exon 140 Dystrophin, Exon 141 Dystrophin, Exon 142 Dystrophin, Exon 143 Dystrophin, Exon 144 Dystrophin, Exon 145 Dystrophin, Exon 146 Dystrophin, Exon 147 Dystrophin, Exon 148 Dystrophin, Exon 149 Dystrophin, Exon 140 Dystrophin, Exon 141 [symbol] Dystrophin, Exon 152 Dystrophin, Exon 44 Dystrophin, Exon 53 Dystrophin, Exon 54 Dystrophin, Exon 55 Dystrophin, Exon 56 Dystrophin, Exon 57 Dystrophin, Exon 58 Dystrophin, Exon 59 Dystrophin, Exon 60 Dystrophin, Exon 61 Dystrophin, Exon 62 Dystrophin, Exon 63 Dystrophin, Exon 64 Dystrophin, Exon 65 Dystrophin, Exon 66 Dystrophin, Exon 67 Dystrophin, Exon 68 Dystrophin, Exon 69 Dystrophin, Exon 70 Dystrophin, Exon 71 Dystrophin, Exon 72 Dystrophin, Exon 73 Dystrophin, Exon 74 Dystrophin, Exon 75 Dystrophin, Exon 76 Dystrophin, Exon 77 Dystrophin, Exon 78 Dystrophin, Exon 79 Dystrophin, Exon 80 Dystrophin, Exon 81 Dystrophin, Exon 82 Dystrophin, Exon 83 Dystrophin, Exon 84 Dystrophin, Exon 85 Dystrophin, Exon 86 Dystrophin, Exon 87 Dystrophin, Exon 88 Dystrophin, Exon 89 Dystrophin, Exon 90 Dystrophin, Exon 91 Dystrophin, Exon 92 Dystrophin, Exon 93 Dystrophin, Exon 94 Dystrophin, Exon 95 Dystrophin, Exon 96 Dystrophin, Exon 97 Dystrophin, Exon 98 Dystrophin, Exon 99 Dystrophin, Fabp3, Factor VII, Factor XI, FAK, FGFR4, FOXP3, FUS, FXII, GFAP, GFP, Glycogen synthase, HINI, HBV, Heat shock protein 47, Heat shock protein 48, Heat shock protein 49, Heat shock protein 50, Heat shock protein 51, Heat shock protein 52, Heat shock protein 53, Heat shock protein 54, Heat shock protein 55, Heat shock protein 56, Heat shock protein 57, Heat shock protein 58, Heat shock protein 59, Helicase, HIV, HOXB13, HPRT, HPV, Hsd11β1, HTT, hydroxyacid oxidase 1, Hydroxysteroid 17β-dehydrogenase 13, Hypoxia-inducible factor 2α, Kinesin spindle protein and vascular endothelial growth factor, KRAS, leader, Leucine-rich repeat kinase 2 (LRRK2), MERS-CoV, MGMT, miR-16, miR-21, MMP-2, MMP-9, MTL-CEBPA, MuRF1, Mycobacterium tuberculosis, COVID-19 N, rabies N 123, rabies N 749, rabies N 903, rabies N1082, rabies N53, rabies N8, Chikungunya Virus ns1 gene, Chikungunya Virus ns2 gene, Chikungunya Virus ns3 gene, Chikungunya Virus ns4 gene, ORF1 b, ORF1a, HIV P24, Rabies P330, P53, Rabies P721, Rabies P91, PCSK9, PDGF, PDL1, COVID-19 PLP, PNPLA3, Polo-like kinase 1, Protein kinase N3, RAF1, RAF-1, RDRP, RSV, SMN2, SNCA, Covid-19 Spike, STAT3, TAU, TGFB1, TGFB1 and Cox2, TMPRSS2, TMPRSS6, TNFα, Transthyretin, VEGF, VEGFR2, VER2, Xanthine dehydrogenase, and YAP1.

95. The modified multi-segmented antisense oligonucleotide of any one of claims 1-90, wherein the modified multi-segmented antisense oligonucleotide is complementary to gene transcript selected from the group consisting of an oncogenic gene transcript, an inflammatory gene transcript, a metabolic disease gene transcript, a cardiovascular gene transcript, a liver [symbol] disease gene transcript, an infectious disease gene transcript, a neurological disease gene transcript, a neuromuscular disease gene transcript, an eye disease gene transcript, a kidney disease gene transcript, a respiratory disease gene transcript, a blood disease gene transcript, a wound healing gene transcript, a transplantation gene transcript, an autoimmune disease gene transcript, and a neuropsychiatric gene transcript.

96. A modified multi-segmented antisense oligonucleotide comprising the structure of the modified multi-segmented antisense oligonucleotide of any one of SEQ ID NOS: 11-1453 or 5000-30983.

97. A modified multi-segmented antisense oligonucleotide comprising the structure of the modified multi-segmented antisense oligonucleotide of any one of SEQ ID NOS: 11-1453 or 5000-30983, or an oligonucleotide having at least 70%, at least 80%, at least 90%, at least 95%, or at least 99% sequence identity thereto.

98. A modified multi-segmented antisense oligonucleotide comprising the structure of the modified multi-segmented antisense oligonucleotide of any one of SEQ ID NOS: 11-1453 or 5000-30983, or an oligonucleotide comprising at least 1, 2, 3, 4 or 5 further modifications thereto.

99. A pharmaceutical composition comprising the modified multi-segmented antisense oligonucleotide of any one of claims 1 to 98, or salt thereof and at least one pharmaceutically acceptable carrier or diluent.

100. A method of treating a disease, disorder, or condition comprising administering to a subject the modified multi-segmented antisense oligonucleotide of any one of claims 1 to 98, or the pharmaceutical composition of claim 99.

101. The method of claim 100, wherein the administering to a subject is parenteral.

102. The method of claim 100, wherein the administering to a subject is subcutaneous.

103. The method of claim 100, wherein the administering to a subject is transdermal.

104. The method of claim 100, wherein the administering to a subject is intraocular.

105. The method of claim 100, wherein the administering to a subject is intramuscular.

106. The method of claim 100, wherein the administering to a subject is intravenous.

107. The method of any one of claims 100 to 106, wherein the subject is human.

108. The method of any one of claims 100 to 106, wherein the subject is a non-human primate.
